(19) United States
(12) Patent Application Publication
(10) Pub. No.: US 2024/0381764 A1
(43) Pub. Date: Nov. 14, 2024

(54) A PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Jeong-Eun YANG, Gyeonggi-do (KR); Ga-Won LEE, Gyeonggi-do (KR); Hee-Ryong KANG, Gyeonggi-do (KR); Hyo-Soon PARK, Gyeonggi-do (KR); Sang-Hee CHO, Gyeonggi-do (KR); Jeong-Hwan JEON, Gyeonggi-do (KR)

(21) Appl. No.: 18/594,285

(22) Filed: Mar. 4, 2024

(30) Foreign Application Priority Data
May 4, 2023 (KR) 10-2023-0058250

(51) Int. Cl. H10K 85/60; C09K 11/06

(52) U.S. Cl. CPC H10K 85/633; C09K 11/06; H10K 85/622; H10K 85/654; H10K 85/6572; H10K 85/6574; H10K 85/6576; C09K 2211/1011; C09K 2211/1014

(57) ABSTRACT

The present disclosure relates to a plurality of host materials, an organic electroluminescent compound, and an organic electroluminescent device comprising the same. An organic electroluminescent device with improved driving voltage, luminous efficiency and/or lifespan properties can be provided by comprising the specific combination of compounds according to the present disclosure as a host material, or by comprising compounds according to the present disclosure.

### TECHNICAL FIELD

The present disclosure relates to a plurality of host materials, an organic electroluminescent compound, and an organic electroluminescent device comprising the same.

### BACKGROUND ART

A small molecular green organic electroluminescent device (OLED) was first developed by Tang, et al., of Eastman Kodak in 1987, utilizing a TPD/ALq3 bi-layer consisting of a light-emitting layer and a charge transport layer. Thereafter, OLED development progressed rapidly, leading to commercialization. Currently, OLEDs primarily use phosphorescent materials with excellent luminous efficiency in panel implementation. However, in various applications such as TVs and lighting, the lifespan of OLEDs is often insufficient, and higher efficiency of OLEDs is still required. Generally, the lifespan of an OLED decreases as its luminance increases. Thus, OLEDs with high luminous efficiency and/or extended lifespan are essential for long-term use and high-resolution displays.

In order to improve luminous efficiency, driving voltage, and/or lifespan, various materials or concepts for an organic layer of an organic electroluminescent device have been proposed, but they were not proven satisfactory in practical use.

In contrast, Korean Patent Application Laid-Open No. 10-2020-0100972 discloses an aromatic amine compound. Nevertheless, the aforementioned reference fails to specifically disclose a particular compound and a specific combination of a plurality of host materials as claimed in the present disclosure. In addition, there is a continuous demand for the development of light-emitting materials with enhanced performance, such as improved driving voltage, luminous efficiency, and/or lifetime properties, as compared to combinations of previously disclosed specific compounds.

### DISCLOSURE OF INVENTION

### Technical Problem

The objective of the present disclosure is to provide a plurality of host materials capable of providing an organic electroluminescent device having lower driving voltage, higher efficiency, and/or extended lifetime properties. Another objective of the present disclosure is to provide an organic electroluminescent compound with a new structure suitable for application to an organic electroluminescent device. Further still, another objective of the present disclosure is to provide an organic electroluminescent device with improved driving voltage, luminous efficiency, and/or lifetime properties by comprising a compound or a specific combination of compounds outlined in the present disclosure.

### Solution to Problem

As a result of intensive study to solve the technical problems, the present inventors found that the above objective can be achieved by a plurality of host materials comprising a first host material comprising a compound represented by the following formula 1, and a second host material comprising a compound represented by the following formula 2:

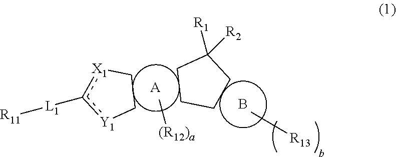

in formula 1,
R₁ and R₂ each independently represent a substituted or unsubstituted (C1-C30)alkyl, a substituted or unsubstituted (C6-C30)aryl, or a substituted or unsubstituted (3- to 30-membered)heteroaryl; or may be linked to each other to form a ring(s);
X₁ and Y₁ each independently represent —N═, —NR₁₄, —O— or —S—, with a proviso that any one of X₁ and Y₁ represents —N═, and the other of X₁ and Y₁ represents —NR₁₄—, —O— or —S—;
ring A and ring B each independently represent a substituted or unsubstituted (C6-C30)arene, or a substituted or unsubstituted (3- to 30-membered)heteroarene;
R₁₁ to R₁₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or the following formula 1-1, with a proviso that at least one of R₁₁ to R₁₄ is represented by the following formula 1-1;

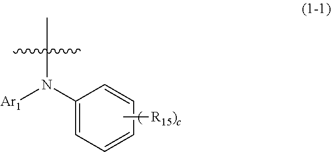

R₁₅ each independently represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₂-N(Ar₂)(Ar₃);
L₁ and L₂ each independently represent a single bond, a substituted or unsubstituted (C6-C30)arylene, or a substituted or unsubstituted (3- to 30-membered)heteroarylene;
Ar₁ to Ar₃ each independently represent hydrogen, deuterium, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; and
a represents an integer of 1 to 6, b represents an integer of 1 to 8, and c represents an integer of 1 to 5, where if a to c are an integer of 2 or more, each of R₁₂, each of R₁₃, and each of R₁₅ may be the same as, or different from, each other.

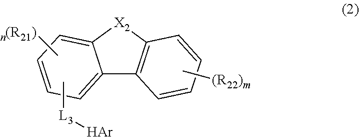

In formula 2,
X₂ represents —O— or —S—;
R₂₁ and R₂₂ each independently represent hydrogen, deuterium, or a substituted or unsubstituted (C6-C30)aryl;
L₃ represents a single bond, or a substituted or unsubstituted (C6-C30)arylene;
HAr represents a substituted or unsubstituted (3- to 30-membered)heteroaryl comprising at least one N atom; and
n represents an integer of 1 to 3, and m represents an integer of 1 to 4, where if n and m are an integer of 2 or more, each of R₂₁ and each of R₂₂ may be the same as, or different from, each other.

In addition, the present inventors found that the above objective can be achieved by an organic electroluminescent compound represented by the following formula 1′.

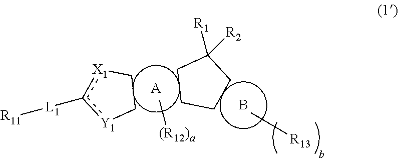

In formula 1′,
R₁ and R₂ each independently represent a substituted or unsubstituted (C1-C30)alkyl, a substituted or unsubstituted (C6-C30)aryl, or a substituted or unsubstituted (3- to 30-membered)heteroaryl; or may be linked to each other to form a ring(s);
X₁ and Y₁ each independently represent —N═, —NR₁₄, —O— or —S—, with a proviso that any one of X₁ and Y₁ represents —N═, and the other of X₁ and Y₁ represents —NR₁₄—, —O— or —S—;
ring A and ring B each independently represent a substituted or unsubstituted (C6-C30)arene, or a substituted or unsubstituted (3- to 30-membered)heteroarene;
R₁₁ and R₁₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or the formula 1′-1 or 1′-2, with a proviso that at least one of R₁₁ to R₁₄ is represented by the following formula 1′-1 or 1′-2, where if R₁₁ is represented by the following formula 1′-1 or 1′-2, R₁₁ is represented by the formula 1′-2;

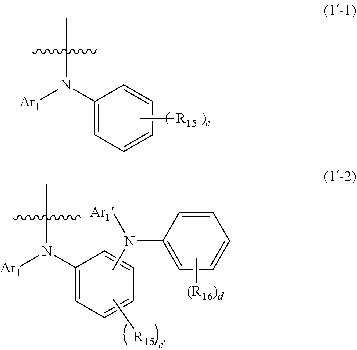

R₁₅ and R₁₆ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₂-N(Ar₂)(Ar₃);
L₁ and L₂ each independently represent a single bond, a substituted or unsubstituted (C6-C30)arylene, or a substituted or unsubstituted (3- to 30-membered)heteroarylene;
Ar₁ to Ar₃ and Ar₁′ each independently represent hydrogen, deuterium, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; and
a represents an integer of 1 to 6, b represents an integer of 1 to 8, c′ represents an integer of 1 to 4, and c and d represent an integer of 1 to 5, where if a to d and c′ are an integer of 2 or more, each of R₁₂, each of R₁₃, each of R₁₅, and each of R₁₆ may be the same as, or different from, each other.

### Advantageous Effects of Invention

An organic electroluminescent device with lower driving voltage, higher luminous efficiency and/or excellent lifespan properties can be provided by comprising the specific combination of compounds according to the present disclosure as a host material, or by comprising compounds according to the present disclosure, and it is possible to manufacture a display system or a lighting system using the same.

### MODE FOR THE INVENTION

Hereinafter, the present disclosure will be described in detail. However, the following description is intended to explain the present disclosure, and is not meant to restrict the scope of the present disclosure.

The term “organic electroluminescent compound” in the present disclosure refers to a compound that may be used in an organic electroluminescent device, and may be incorporated into any layer constituting an organic electroluminescent device, as necessary.

The term “organic electroluminescent material” in the present disclosure refers to a material that may be used in an organic electroluminescent device, and may comprise at least one compound. The organic electroluminescent material may be incorporated into any layer constituting an organic electroluminescent device, as necessary. For example, the organic electroluminescent material may be any of the following: a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc. The hole transport zone material may be at least one selected from the group consisting of a hole transport material, a hole injection material, an electron blocking material, a hole auxiliary material, and a light-emitting auxiliary material.

The term “a plurality of organic electroluminescent materials” in the present disclosure refers to an organic electroluminescent material(s) comprising a combination of two or more compounds, which may be incorporated into any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of two or more compounds that may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. The two or more compounds may be comprised in the same layer or different layers, and may be mixture-evaporated or co-evaporated, or may be individually evaporated.

The term “a plurality of host materials” in the present disclosure means an organic electroluminescent material(s) comprising a combination of two or more host materials. It may mean both a material before being comprised in an organic electroluminescent device (for example, before vapor deposition) and a material after being comprised in an organic electroluminescent device (for example, after vapor deposition). The plurality of host materials of the present disclosure may be comprised in any light-emitting layer constituting an organic electroluminescent device, wherein the two or more compounds comprised in the plurality of host materials may be comprised together in one light-emitting layer or may be respectively comprised in different light-emitting layers. When two or more host materials are comprised in one layer, for example, they may be mixture-evaporated to form a layer, or separately and simultaneously co-evaporated to form a layer.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C2-C30)alkenyl” in the present disclosure is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” in the present disclosure is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a monocyclic or polycyclic hydrocarbon having 3 to 30 ring backbone carbon atoms, preferably 3 to 20 ring backbone carbon atoms, and more preferably 3 to 7 ring backbone carbon atoms. Examples of the cycloalkyl may include cyclopropyl, cyclobutyl, cyclopentyl, cyclohexyl, cyclopentylmethyl, cyclohexylmethyl, etc. The term “(3- to 7-membered)heterocycloalkyl” in the present disclosure is meant to be a cycloalkyl having 3 to 7 ring backbone atoms, preferably 5 to 7 ring backbone atoms and containing at least one heteroatom(s) selected from the group consisting of B, N, O, S, Si, and P, preferably the group consisting of O, S, and N. The above heterocycloalkyl includes tetrahydrofuran, pyrrolidine, thiolane, tetrahydropyran, etc. The term “(C6-C30)aryl(ene)” and “(C6-C30)arene” in the present disclosure is meant to be a monocyclic or fused ring radical derived from an aromatic hydrocarbon having 6 to 30 ring backbone carbon atoms. The number of ring backbone carbon atoms is preferably 6 to 25, and more preferably 6 to 18. The above aryl may be partially saturated, and may comprise a spiro structure. The above aryl may include any of the following: phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, tetramethyldihydrophenanthrenyl, etc. More specifically, the aryl may include any of the following: phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenylyl, 4″-t-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” and “(3- to 30-membered)heteroarene” in the present disclosure is meant to be an aryl(en) group having 3 to 30 ring backbone atoms and including at least one, preferably 1 to 4, heteroatom(s) selected from the group consisting of B, N, O, S, Si, P, Se, Te, and Ge. It may be a monocyclic ring or a fused ring condensed with at least one benzene ring, and may be partially saturated. In addition, the above heteroaryl(ene) comprises one formed by linking at least one heteroaryl or aryl group to a heteroaryl(ene) group via a single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, benzophenanthrofuranyl, dibenzothiophenyl, benzimidazolyl, benzothiazolyl, benzoisothiazolyl, benzophenanthrothiophenyl, benzoisoxazolyl, benzoxazolyl, phenanthroxazolyl, phenanthrothiazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may include any of the following: 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. The term “a fused ring group of a (C3-C30)aliphatic ring(s) and a (C6-C30) aromatic ring(s)” is meant to be a functional group in which at least one aliphatic ring(s) having 3 to 30 ring backbone carbon atoms, preferably 3 to 25 ring backbone carbon atoms, and more preferably 3 to 18 ring backbone carbon atoms is fused with at least one aromatic ring(s) having 6 to 30 ring backbone carbon atoms, preferably 6 to 25 ring backbone carbon atoms, and more preferably 6 to 18 ring backbone carbon atoms. For example, the fused ring group may include a fused ring group of at least one benzene and at least one cyclohexane, or a fused ring group of at least one naphthalene and at least one cyclopentane, etc. In the present disclosure, the carbon atom of the fused ring group of a (C3-C30) aliphatic ring(s) and a (C6-C30) aromatic ring(s) may be replaced with at least one heteroatom(s) selected from B, N, O, S, Si, and P, preferably at least one heteroatom(s) selected from N, O, and S. In the present disclosure, “halogen” includes F, Cl, Br, and I.

In addition, “ortho (o-),” “meta (m-),” and “para (p-)” are prefixes, which represent the relative positions of substituents. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

The term “a ring formed by a linkage of adjacent substituents” means that at least two adjacent substituents are linked to or fused with each other to form a substituted or unsubstituted mono- or polycyclic (3- to 30-membered) alicyclic or aromatic ring, or the combination thereof, preferably a substituted or unsubstituted mono- or polycyclic (5- to 25-membered) alicyclic or aromatic ring, or the combination thereof. In addition, the formed ring may contain at least one heteroatom selected from B, N, O, S, Si, and P, and preferably at least one heteroatom selected from N, O, and S. According to one embodiment of the present disclosure, the number of ring backbone atoms is 5 to 20, and according to another embodiment of the present disclosure, the number of ring backbone atoms is 5 to 15.

In addition, “substituted” in the expression “substituted or unsubstituted” means that a hydrogen atom in a certain functional group is replaced with another atom or another functional group (i.e., a substituent), and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as substituents in which two heteroaryls are linked. Herein, the substituent(s) of the substituted alkyl, the substituted aryl(ene), the substituted heteroaryl(ene), the substituted arene, the substituted heteroarene, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s) and the substituted alkenyl in the formulas of the present disclosure each independently are at least one selected from the group consisting of the following: deuterium, a halogen, a cyano, a carboxyl, a nitro, a hydroxy, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (5- to 30-membered)heteroaryl unsubstituted or substituted with a (C6-C30)aryl(s), a (C6-C30)aryl unsubstituted or substituted with a (5- to 30-membered)heteroaryl(s), a tri(C1-C30)alkylsilyl, a tri(C6-C30)arylsilyl, a di(C1-C30)alkyl(C6-C30)arylsilyl, a (C1-C30)alkyldi(C6-C30)arylsilyl, a fused ring group of a (C3-C30) aliphatic ring(s) and a (C6-C30) aromatic ring(s), an amino, a mono- or di-(C1-C30)alkylamino, a mono- or di-(C2-C30)alkenylamino, a (C1-C30)alkyl(C2-C30)alkenylamino, a substituted or unsubstituted mono- or di-(C6-C30)arylamino, a (C1-C30)alkyl(C6-C30)arylamino, a mono- or di-(3- to 30-membered)heteroarylamino, a (C1-C30)alkyl(3- to 30-membered)heteroarylamino, a (C2-C30)alkenyl(C6-C30)arylamino, a (C2-C30)alkenyl(3- to 30-membered)heteroarylamino, a (C6-C30)aryl(3- to 30-membered)heteroarylamino, a (C1-C30)alkylcarbonyl, a (C1-C30)alkoxycarbonyl, a (C6-C30)arylcarbonyl, a (C6-C30)arylphospinyl, a di(C6-C30)arylboronyl, a di(C1-C300alkylboronyl, a (C1-C30)alkyl(C6-C30)arylboronyl, a (C6-C30)aryl(C1-C30)alkyl, and a (C1-C30)alkyl(C6-C30)aryl. According to one embodiment of the present disclosure, the substituent(s), each independently, may be a methyl, a phenyl, a biphenyl, a terphenyl, a naphthyl, a chrysenyl, a phenyl substituted with a chrysenyl(s) substituted with a phenyl(s), a phenyl substituted with a naphthyl(s), a naphthyl substituted with a chrysenyl(s), a naphthyl substituted with a biphenyl(s), a naphthyl substituted with a phenyl(s), a naphthyl substituted with a naphthyl(s), a chrysenyl substituted with a phenyl(s), a chrysenyl substituted with a biphenyl(s), a dibenzofuranyl, etc.

The plurality of host materials according to the present disclosure comprises a first host material comprising the compound represented by formula 1 and a second host material comprising the compound represented by formula 2, and may be comprised in a light-emitting layer of the organic electroluminescent device according to the present disclosure.

Hereinafter, the compound represented by formula 1 will be described in more detail.

In formula 1, R₁ and R₂ each independently represent a substituted or unsubstituted (C1-C30)alkyl, a substituted or unsubstituted (C6-C30)aryl, or a substituted or unsubstituted (3- to 30-membered)heteroaryl; or may be linked to each other to form a ring(s). For example, R₁ and R₂ each independently may be a methyl, etc.

In formula 1, X₁ and Y₁ each independently represent —N═, —NR₁₄, —O— or —S—, with a proviso that any one of X₁ and Y₁ represents —N═, and the other of X₁ and Y₁ represents —NR₁₄—, —O— or —S—. For example, X₁ may be —N═, Y₁ may be —NR₁₄, —O—, —S—, etc.

In formula 1, ring A and ring B each independently represent a substituted or unsubstituted (C6-C30)arene, or a substituted or unsubstituted (3- to 30-membered)heteroarene. According to one embodiment of the present disclosure, ring A and ring B each independently may be a substituted or unsubstituted (C6-C18)arene. According to one embodiment of the present disclosure, ring A and ring B each independently may be an unsubstituted (C6-C18)arene. For example, ring A and ring B each independently may be a benzene, a naphthalene, a phenanthrene, etc.

In formula 1, R₁₁ to R₁₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or the following formula 1-1, with a proviso that at least one of R₁₁ to R₁₄ is represented by the following formula 1-1. According to one embodiment of the present disclosure, R₁₁ to R₁₄ each independently may be hydrogen, a substituted or unsubstituted (C6-C25)aryl, or the following formula 1-1. For example, R₁₁ to R₁₄ each independently may be hydrogen, a naphthyl, a phenyl unsubstituted or substituted with a methyl(s) or an ethylenyl(s), the following formula 1-1, etc.

According to one embodiment of the present disclosure, formula 1 may be represented by the following formula 1-1.

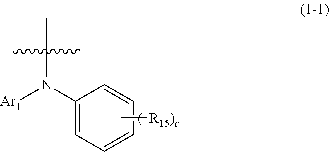

In formula 1-1, R₁₅ each independently represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₂-N(Ar₂)(Ar₃). According to one embodiment of the present disclosure, R₁₅ each independently may be hydrogen, a substituted or unsubstituted (C6-C18)aryl, -L₂-N(Ar₂)(Ar₃). For example, R₁₅ each independently may be hydrogen, a phenyl, -L₂-N(Ar₂)(Ar₃), etc.

In formula 1 and 1-1, L₁ and L₂ each independently represent a single bond, a substituted or unsubstituted (C6-C30)arylene, or a substituted or unsubstituted (3- to 30-membered)heteroarylene. According to one embodiment of the present disclosure, L₁ and L₂ each independently may be a single bond, or a substituted or unsubstituted (C6-C18)arylene. For example, L₁ and L₂ each independently may be a single bond, phenylene, etc.

In formula 1-1, Ar₁ to Ar₃ each independently represent hydrogen, deuterium, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl. According to one embodiment of the present disclosure, Ar₁ to Ar₃ each independently may be a substituted or unsubstituted (C6-C25)aryl, or a substituted or unsubstituted (3- to 25-membered)heteroaryl. For example, Ar₁ to Ar₃ each independently may be a phenyl, a dibenzofuranyl, a carbazolyl substituted with a phenyl(s), a dibenzothiophenyl, a naphthyl, etc.

In formula 1 and 1-1, a represents an integer of 1 to 6, b represents an integer of 1 to 8, and c represents an integer of 1 to 5, where if a to c are an integer of 2 or more, each of R₁₂, each of R₁₃, and each of R₁₅ may be the same as, or different from, each other.

Hereinafter, the compound represented by formula 2 will be described in more detail.

In formula 2, X₂ represents —O— or —S—. For example, X₂ may be —O— or —S—.

In formula 2, R₂₁ and R₂₂ each independently represent hydrogen, deuterium, or a substituted or unsubstituted (C6-C30)aryl. According to one embodiment of the present disclosure, R₂₁ and R₂₂ each independently may be hydrogen, a substituted or unsubstituted (C6-C25)aryl. For example, R₂₁ and R₂₂ each independently may be hydrogen, an anthracenyl, a biphenyl, a naphthyl unsubstituted or substituted with a phenyl(s), a phenyl unsubstituted or substituted with a naphthyl(s), etc.

In formula 2, L₃ represents a single bond, or a substituted or unsubstituted (C6-C30)arylene. For example, L₃ may be a single bond.

In formula 2, HAr represents a substituted or unsubstituted (3- to 30-membered)heteroaryl comprising at least one N atom. According to one embodiment of the present disclosure, HAr may be a substituted or unsubstituted (3- to 25-membered)heteroaryl comprising at least one N atom. According to another embodiment of the present disclosure, HAr may be a substituted or unsubstituted (3- to 18-membered)heteroaryl comprising three N atoms. According to another embodiment of the present disclosure, HAr may be a substituted or unsubstituted 6-membered heteroaryl comprising three N atoms. For example, HAr may be a triazinyl substituted with a chrysenyl(s), a triazinyl substituted with a chrysenyl(s) and a phenyl(s), a triazinyl substituted with a chrysenyl(s) and a biphenyl(s), a triazinyl substituted with a chrysenyl(s) and a naphthyl(s), a triazinyl substituted with a chrysenyl(s) substituted with a phenyl(s) and a phenyl(s), a triazinyl substituted with a chrysenyl(s) substituted with a biphenyl(s) and a phenyl(s), a triazinyl substituted with a dibenzofuranyl(s) and a phenyl(s), a triazinyl substituted with a dibenzofuranyl(s) and a naphthyl(s), a triazinyl substituted with a naphthyl(s), a triazinyl substituted with a naphthyl(s) and phenyl(s), a triazinyl substituted with a naphthyl(s) and a biphenyl(s), a triazinyl substituted with a naphthyl(s) and a terphenyl(s), a triazinyl substituted with a naphthyl(s) and a phenyl(s) substituted with a naphthyl(s), a triazinyl substituted with a naphthyl(s) and a naphthyl(s) substituted with a naphthyl(s), a triazinyl substituted with a naphthyl(s) substituted with a phenyl(s) and a naphthyl(s), a triazinyl substituted with a naphthyl(s) substituted with a biphenyl(s) and a phenyl(s), a triazinyl substituted with a naphthyl(s) substituted with a chrysenyl(s) and a phenyl(s), a triazinyl substituted with a phenyl(s) and a naphthyl(s) substituted with a naphthyl(s), a triazinyl substituted with a phenyl(s) substituted with a chrysenyl(s) substituted with a phenyl(s) and a phenyl(s), a triazinyl substituted with a phenyl(s) substituted with a naphthyl(s) and a naphthyl(s) substituted with a phenyl(s), a triazinyl substituted with a terphenyl(s) and a phenyl(s).
n represents an integer of 1 to 3, and m represents an integer of 1 to 4, where if n and m are an integer of 2 or more, each of R₂₁ and each of R₂₂ may be the same as, or different from, each other.

According to one embodiment of the present disclosure, the above formula 1 may be represented by one of the following formulas 3 and 4.

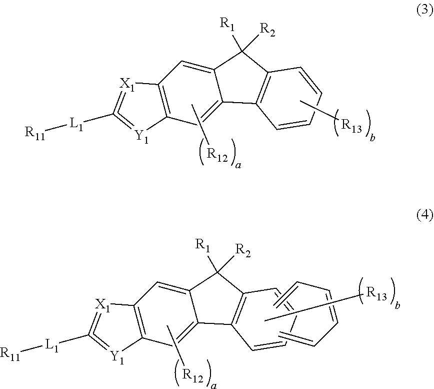

In formulas 3 and 4,
R₁, R₂, R₁₁ to R₁₃, X₁, Y₁, and L₁, are as defined in formula 1, a represents an integer of 1 or 2, and b represents an integer of 1 to 6.

According to one embodiment of the present disclosure, the above formula 2 is a plurality of host materials represented by one of the following formulas 5 to 8.

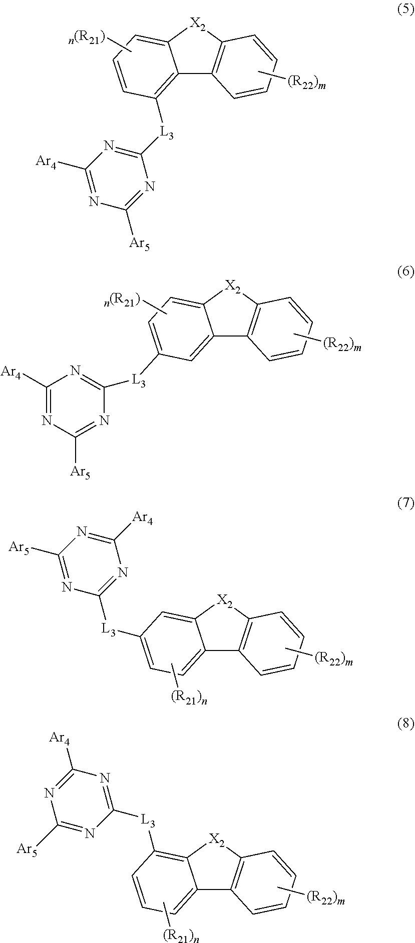

In formulas 5 to 8,
Ar₄ and Ar₅ each independently represent a substituted or unsubstituted (C6-C30)aryl, or a substituted or unsubstituted (3- to 30-membered)heteroaryl. According to one embodiment of the present disclosure, Ar₄ and Ar₅ each independently represent a substituted or unsubstituted (C6-C25)aryl, or a substituted or unsubstituted (3- to 25-membered)heteroaryl. According to another embodiment of the present disclosure, Ar₄ and Ar₅ each independently represent a substituted or unsubstituted (C6-C18)aryl, or a substituted or unsubstituted (3- to 18-membered)heteroaryl. For example, Ar₄ and Ar₅ each independently may be a chrysenyl substituted with a phenyl(s), a phenyl unsubstituted or substituted with a naphthyl(s), a phenyl, a biphenyl, a naphthyl, a naphthyl substituted with a chrysenyl(s), a chrysenyl unsubstituted or substituted with a phenyl(s) or a biphenyl(s), a biphenyl, a terphenyl, a dibenzofuranyl, etc.
X₂, R₂₁ and R₂₂, L₃, n, m are as defined in formula 2.

According to one embodiment of the present disclosure, the compound represented by formula 1 may be at least one selected from the following compounds.

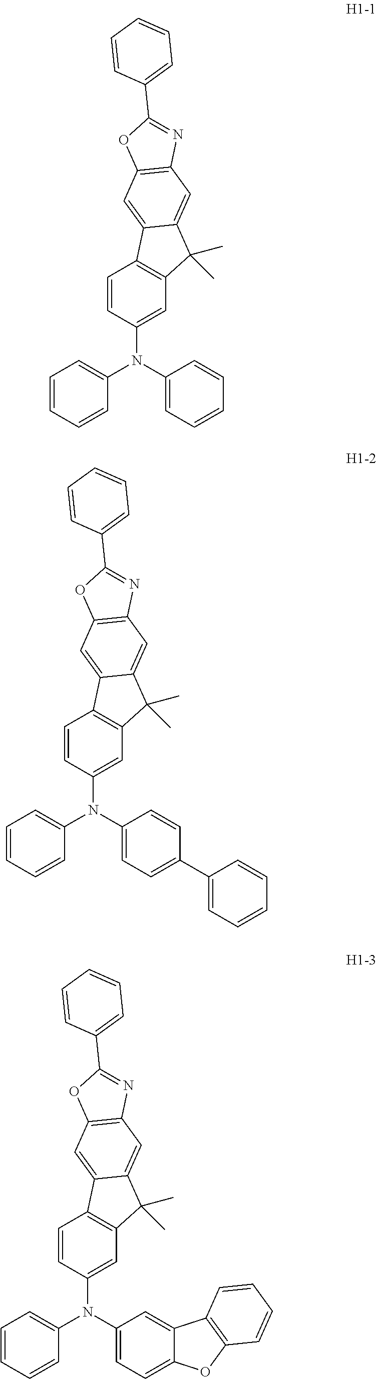

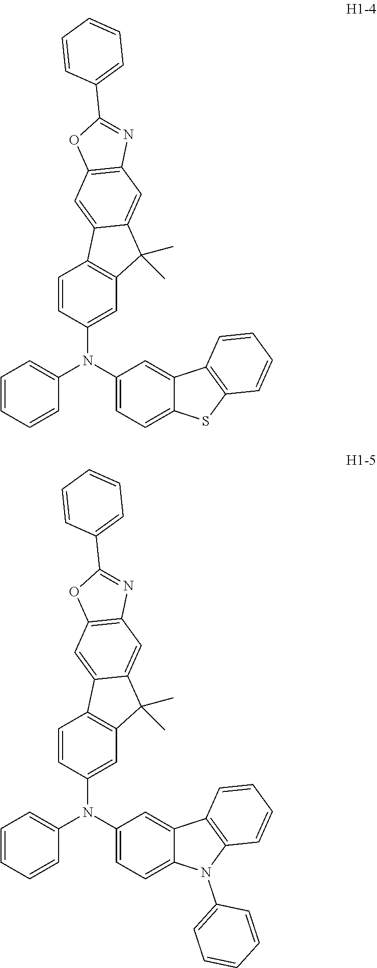 [SMILES: CC1(C)c2cc(N(c3ccccc3)c3ccc4sc5ccccc5c4c3)ccc2-c2cc3oc(-c4ccccc4)nc3cc21 ; CC1(C)c2cc(N(c3ccccc3)c3ccc4c(c3)c3ccccc3n4-c3ccccc3)ccc2-c2cc3oc(-c4ccccc4)nc3cc21]

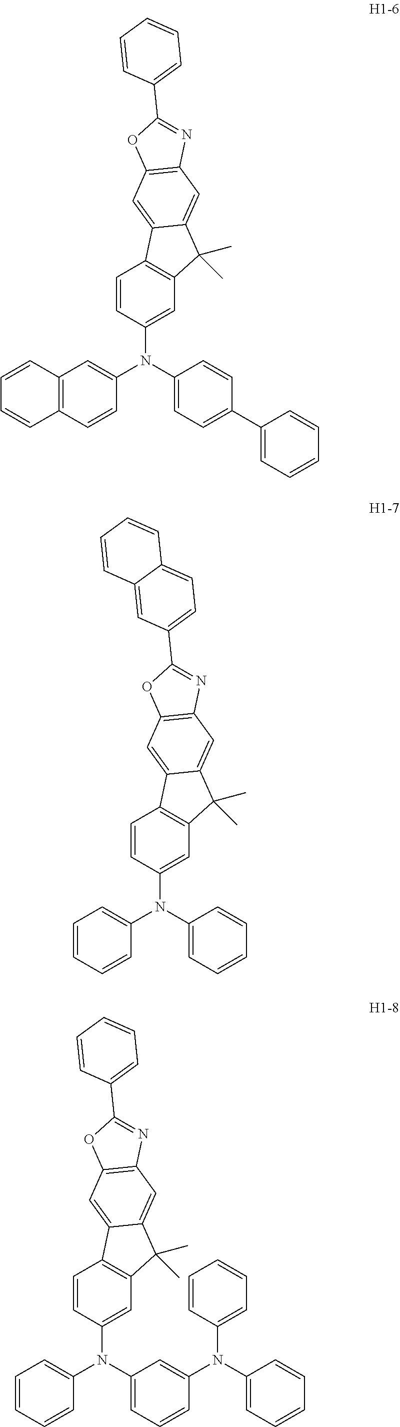

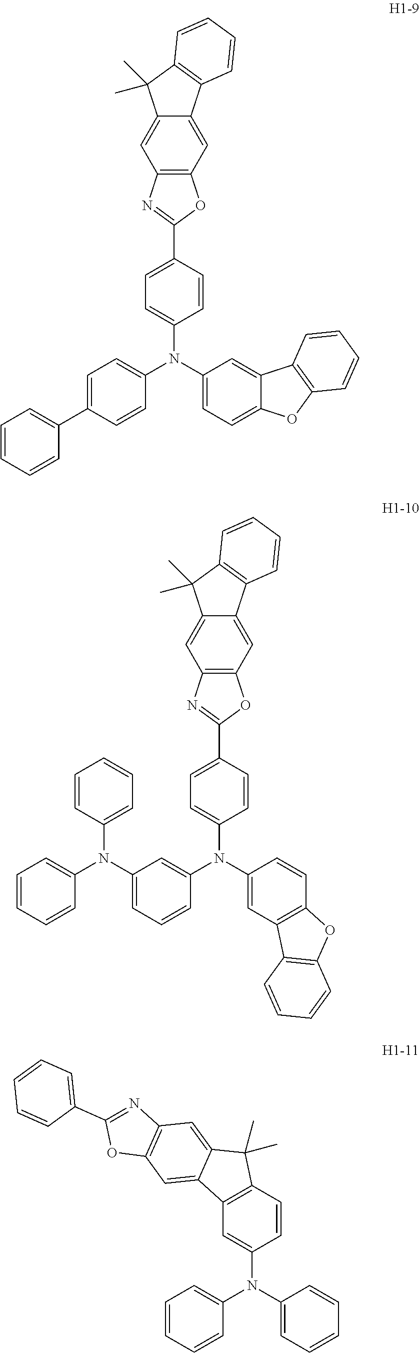 [SMILES: CC1(C)c2ccccc2-c2cc3oc(-c4ccc(N(c5ccc(-c6ccccc6)cc5)c5ccc6oc7ccccc7c6c5)cc4)nc3cc21 ; CC1(C)c2ccccc2-c2cc3oc(-c4ccc(N(c5cccc(N(c6ccccc6)c6ccccc6)c5)c5ccc6oc7ccccc7c6c5)cc4)nc3cc21 ; CC1(C)c2ccc(N(c3ccccc3)c3ccccc3)cc2-c2cc3oc(-c4ccccc4)nc3cc21]

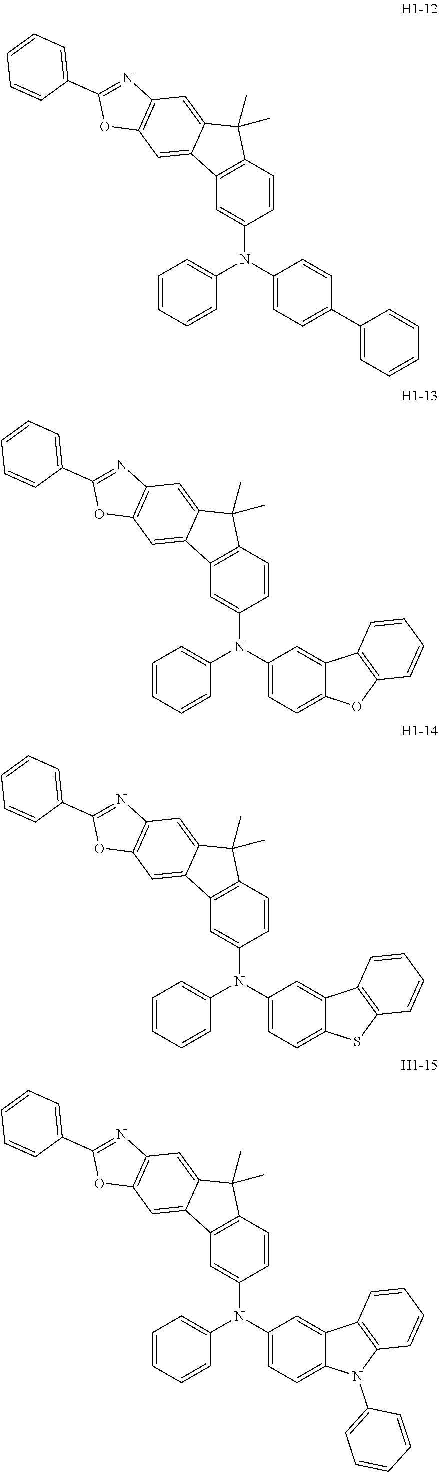 [SMILES: CC1(C)c2ccc(N(c3ccccc3)c3ccc(-c4ccccc4)cc3)cc2-c2cc3oc(-c4ccccc4)nc3cc21 ; CC1(C)c2ccc(N(c3ccccc3)c3ccc4oc5ccccc5c4c3)cc2-c2cc3oc(-c4ccccc4)nc3cc21 ; CC1(C)c2ccc(N(c3ccccc3)c3ccc4sc5ccccc5c4c3)cc2-c2cc3oc(-c4ccccc4)nc3cc21 ; CC1(C)c2ccc(N(c3ccccc3)c3ccc4c(c3)c3ccccc3n4-c3ccccc3)cc2-c2cc3oc(-c4ccccc4)nc3cc21]

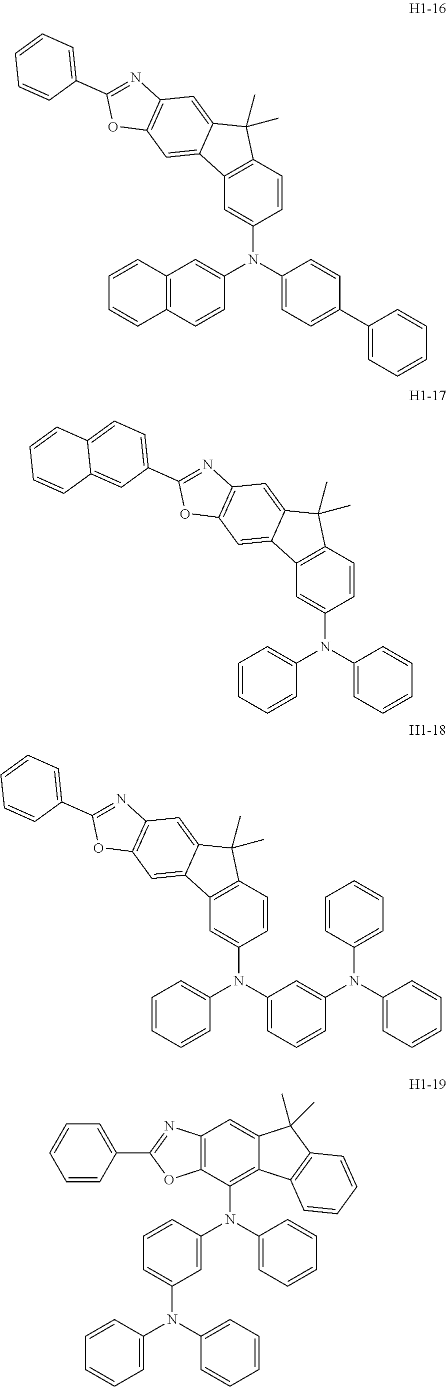 [SMILES: CC1(C)c2ccc(N(c3ccc(-c4ccccc4)cc3)c3ccc4ccccc4c3)cc2-c2cc3oc(-c4ccccc4)nc3cc21 ; CC1(C)c2ccc(N(c3ccccc3)c3ccccc3)cc2-c2cc3oc(-c4ccc5ccccc5c4)nc3cc21 ; CC1(C)c2ccc(N(c3ccccc3)c3cccc(N(c4ccccc4)c4ccccc4)c3)cc2-c2cc3oc(-c4ccccc4)nc3cc21 ; CC1(C)c2ccccc2-c2c1cc1nc(-c3ccccc3)oc1c2N(c1ccccc1)c1cccc(N(c2ccccc2)c2ccccc2)c1]

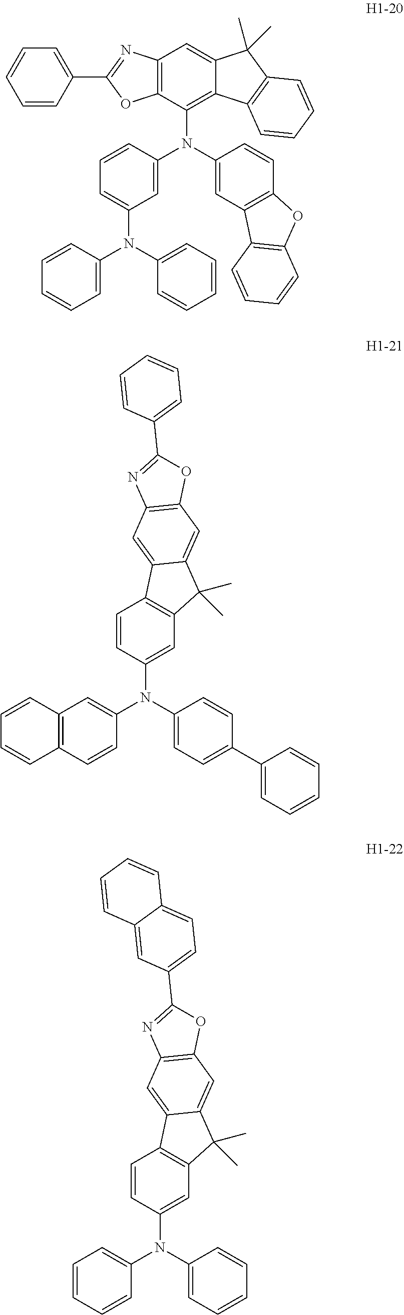 [SMILES: CC1(C)c2ccccc2-c2c1cc1nc(-c3ccccc3)oc1c2N(c1cccc(N(c2ccccc2)c2ccccc2)c1)c1ccc2oc3ccccc3c2c1 ; CC1(C)c2cc(N(c3ccc(-c4ccccc4)cc3)c3ccc4ccccc4c3)ccc2-c2cc3nc(-c4ccccc4)oc3cc21 ; CC1(C)c2cc(N(c3ccccc3)c3ccccc3)ccc2-c2cc3nc(-c4ccc5ccccc5c4)oc3cc21]

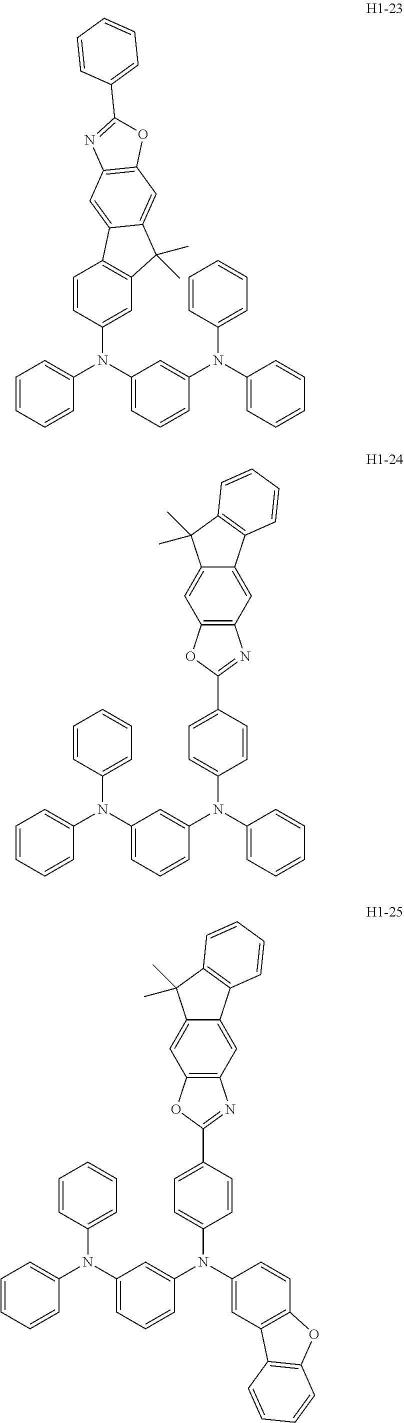 [SMILES: CC1(C)c2cc(N(c3ccccc3)c3cccc(N(c4ccccc4)c4ccccc4)c3)ccc2-c2cc3nc(-c4ccccc4)oc3cc21 ; CC1(C)c2ccccc2-c2cc3nc(-c4ccc(N(c5ccccc5)c5cccc(N(c6ccccc6)c6ccccc6)c5)cc4)oc3cc21 ; CC1(C)c2ccccc2-c2cc3nc(-c4ccc(N(c5cccc(N(c6ccccc6)c6ccccc6)c5)c5ccc6oc7ccccc7c6c5)cc4)oc3cc21]

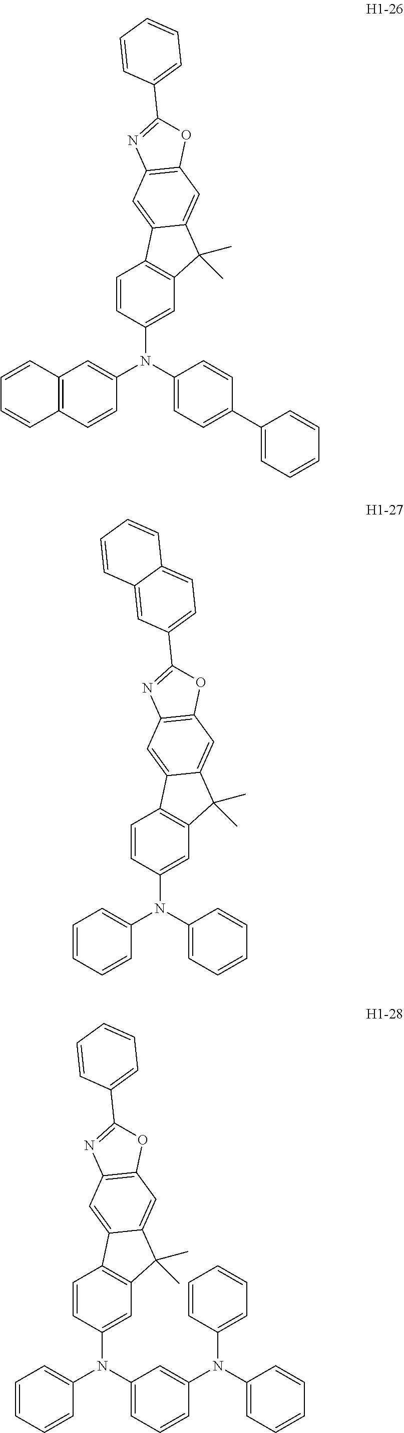 [SMILES: CC1(C)c2cc(N(c3ccc(-c4ccccc4)cc3)c3ccc4ccccc4c3)ccc2-c2cc3nc(-c4ccccc4)oc3cc21 ; CC1(C)c2cc(N(c3ccccc3)c3ccccc3)ccc2-c2cc3nc(-c4ccc5ccccc5c4)oc3cc21 ; CC1(C)c2cc(N(c3ccccc3)c3cccc(N(c4ccccc4)c4ccccc4)c3)ccc2-c2cc3nc(-c4ccccc4)oc3cc21]

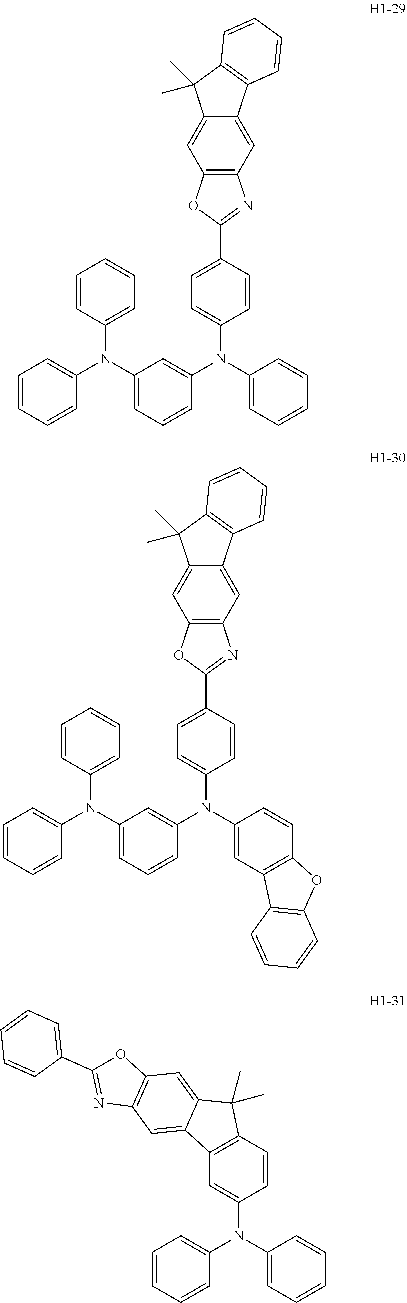 [SMILES: CC1(C)c2ccccc2-c2cc3nc(-c4ccc(N(c5ccccc5)c5cccc(N(c6ccccc6)c6ccccc6)c5)cc4)oc3cc21 ; CC1(C)c2ccccc2-c2cc3nc(-c4ccc(N(c5cccc(N(c6ccccc6)c6ccccc6)c5)c5ccc6oc7ccccc7c6c5)cc4)oc3cc21 ; CC1(C)c2ccc(N(c3ccccc3)c3ccccc3)cc2-c2cc3nc(-c4ccccc4)oc3cc21]

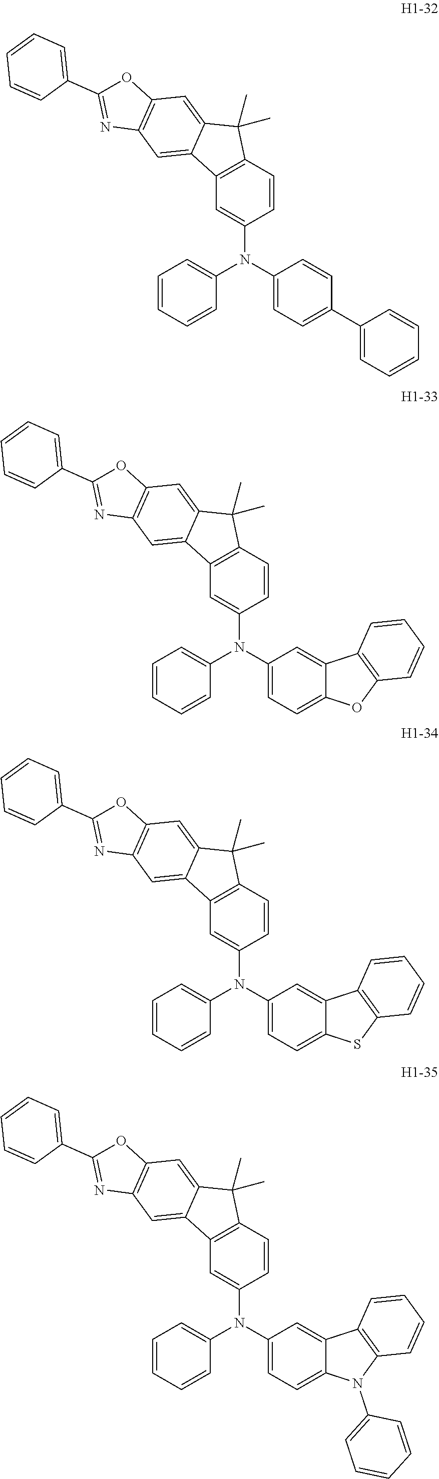 [SMILES: CC1(C)c2ccc(N(c3ccccc3)c3ccc(-c4ccccc4)cc3)cc2-c2cc3nc(-c4ccccc4)oc3cc21 ; CC1(C)c2ccc(N(c3ccccc3)c3ccc4oc5ccccc5c4c3)cc2-c2cc3nc(-c4ccccc4)oc3cc21 ; CC1(C)c2ccc(N(c3ccccc3)c3ccc4sc5ccccc5c4c3)cc2-c2cc3nc(-c4ccccc4)oc3cc21 ; CC1(C)c2ccc(N(c3ccccc3)c3ccc4c(c3)c3ccccc3n4-c3ccccc3)cc2-c2cc3nc(-c4ccccc4)oc3cc21]

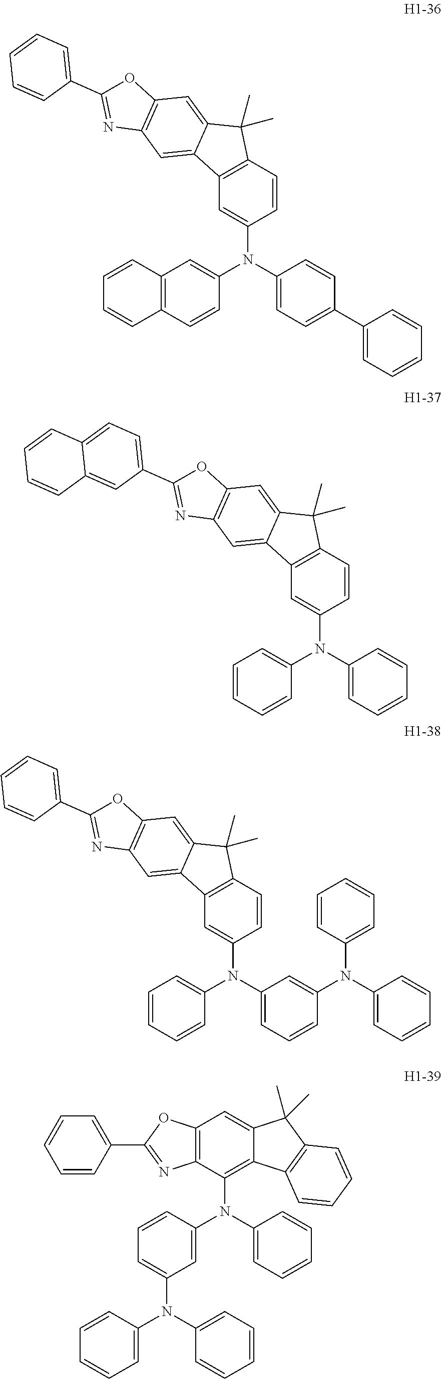

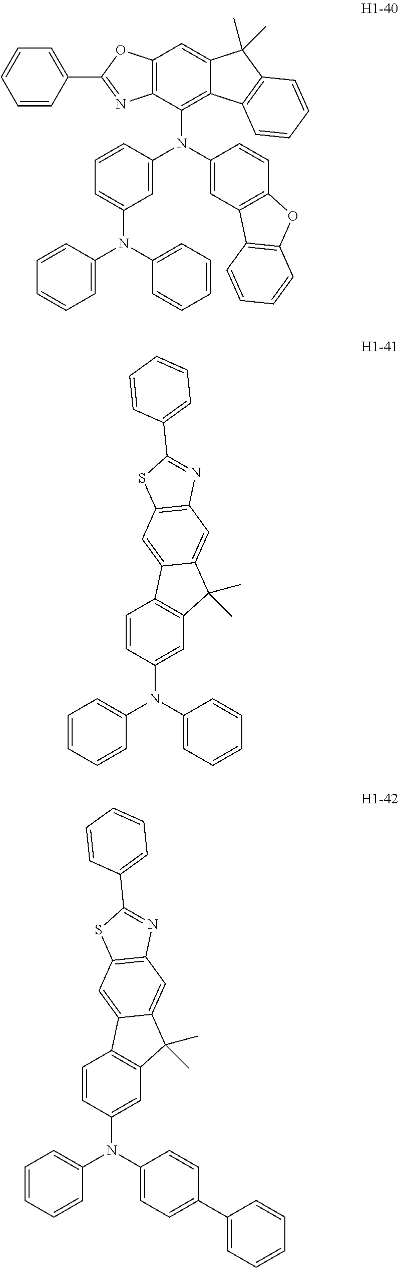

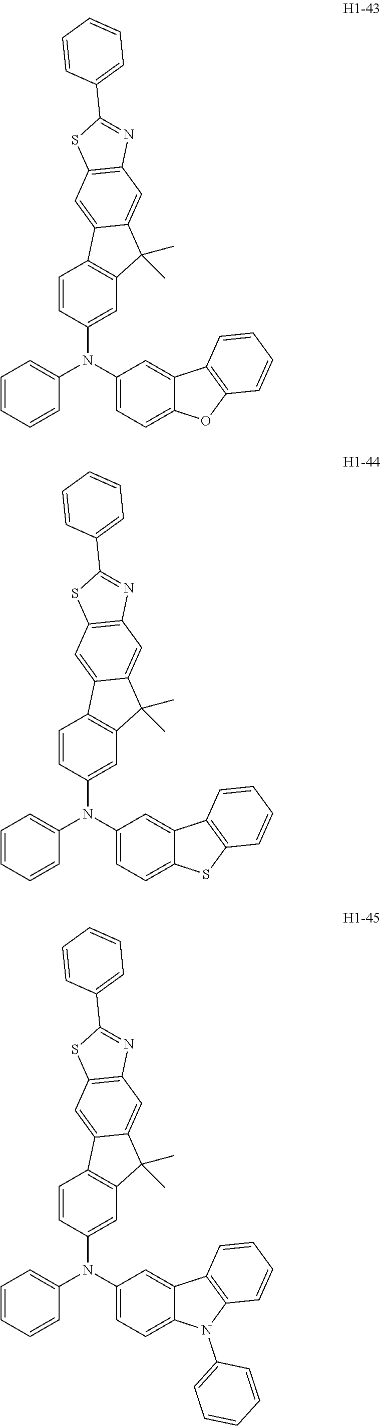 [SMILES: CC1(C)c2cc(N(c3ccccc3)c3ccc4oc5ccccc5c4c3)ccc2-c2cc3sc(-c4ccccc4)nc3cc21 ; CC1(C)c2cc(N(c3ccccc3)c3ccc4sc5ccccc5c4c3)ccc2-c2cc3sc(-c4ccccc4)nc3cc21 ; CC1(C)c2cc(N(c3ccccc3)c3ccc4c(c3)c3ccccc3n4-c3ccccc3)ccc2-c2cc3sc(-c4ccccc4)nc3cc21]

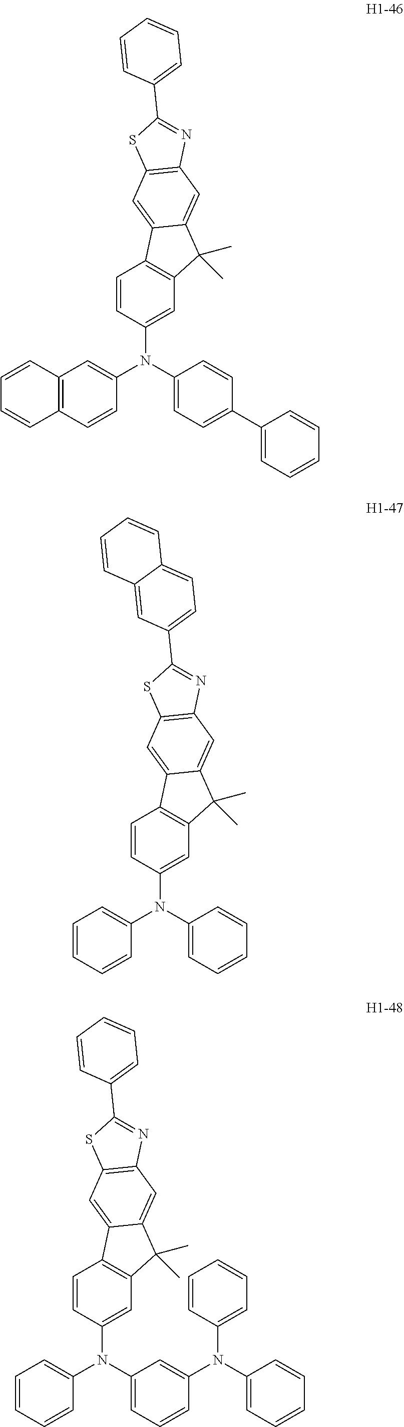

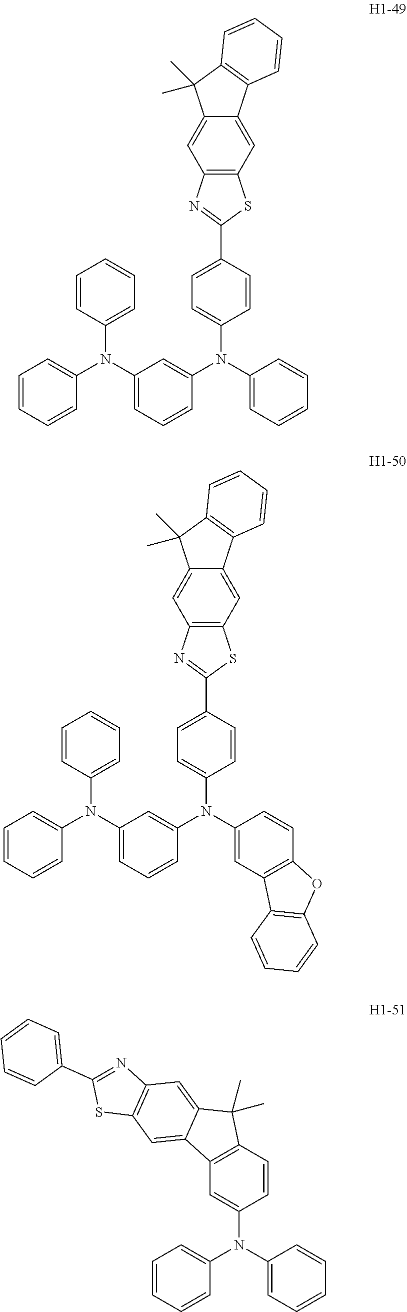 [SMILES: CC1(C)c2ccccc2-c2cc3sc(-c4ccc(N(c5ccccc5)c5cccc(N(c6ccccc6)c6ccccc6)c5)cc4)nc3cc21 ; CC1(C)c2ccccc2-c2cc3sc(-c4ccc(N(c5cccc(N(c6ccccc6)c6ccccc6)c5)c5ccc6oc7ccccc7c6c5)cc4)nc3cc21 ; CC1(C)c2ccc(N(c3ccccc3)c3ccccc3)cc2-c2cc3sc(-c4ccccc4)nc3cc21]

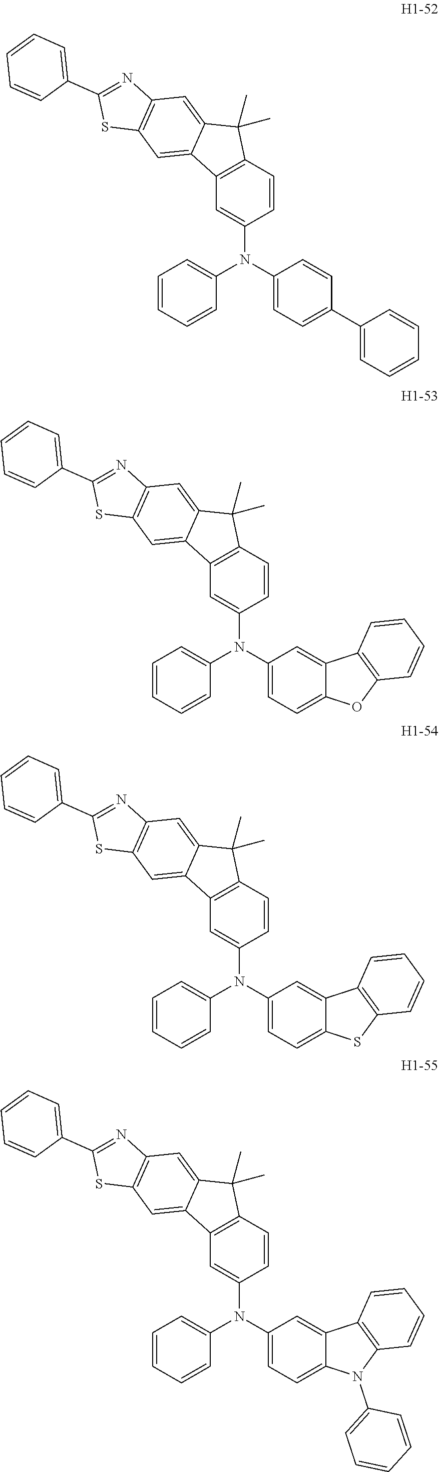 [SMILES: CC1(C)c2ccc(N(c3ccccc3)c3ccc(-c4ccccc4)cc3)cc2-c2cc3sc(-c4ccccc4)nc3cc21 ; CC1(C)c2ccc(N(c3ccccc3)c3ccc4oc5ccccc5c4c3)cc2-c2cc3sc(-c4ccccc4)nc3cc21 ; CC1(C)c2ccc(N(c3ccccc3)c3ccc4sc5ccccc5c4c3)cc2-c2cc3sc(-c4ccccc4)nc3cc21 ; CC1(C)c2ccc(N(c3ccccc3)c3ccc4c(c3)c3ccccc3n4-c3ccccc3)cc2-c2cc3sc(-c4ccccc4)nc3cc21]

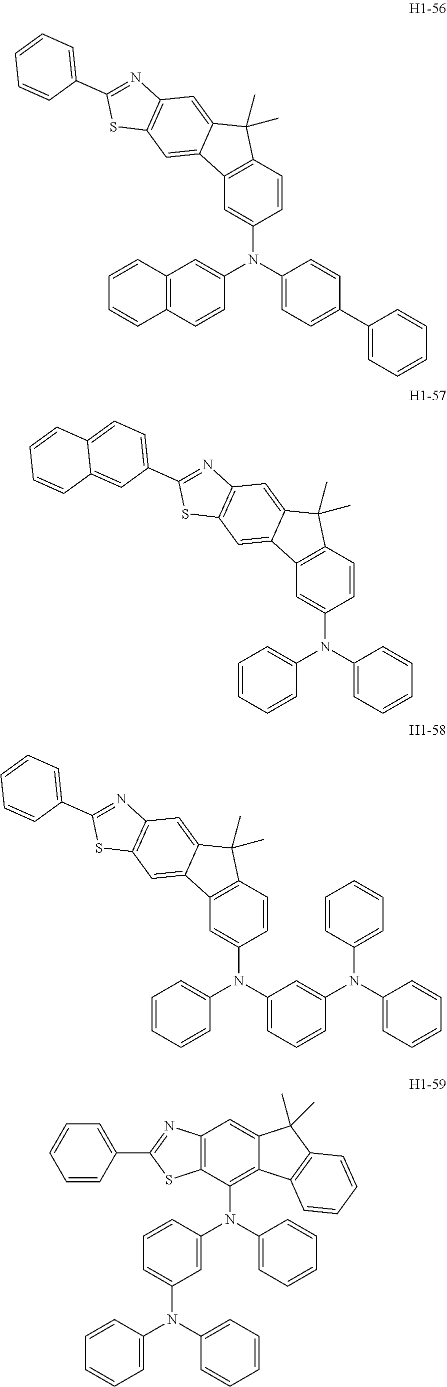 [SMILES: CC1(C)c2ccc(N(c3ccc(-c4ccccc4)cc3)c3ccc4ccccc4c3)cc2-c2cc3sc(-c4ccccc4)nc3cc21 ; CC1(C)c2ccc(N(c3ccccc3)c3ccccc3)cc2-c2cc3sc(-c4ccc5ccccc5c4)nc3cc21 ; CC1(C)c2ccc(N(c3ccccc3)c3cccc(N(c4ccccc4)c4ccccc4)c3)cc2-c2cc3sc(-c4ccccc4)nc3cc21 ; CC1(C)c2ccccc2-c2c1cc1nc(-c3ccccc3)sc1c2N(c1ccccc1)c1cccc(N(c2ccccc2)c2ccccc2)c1]

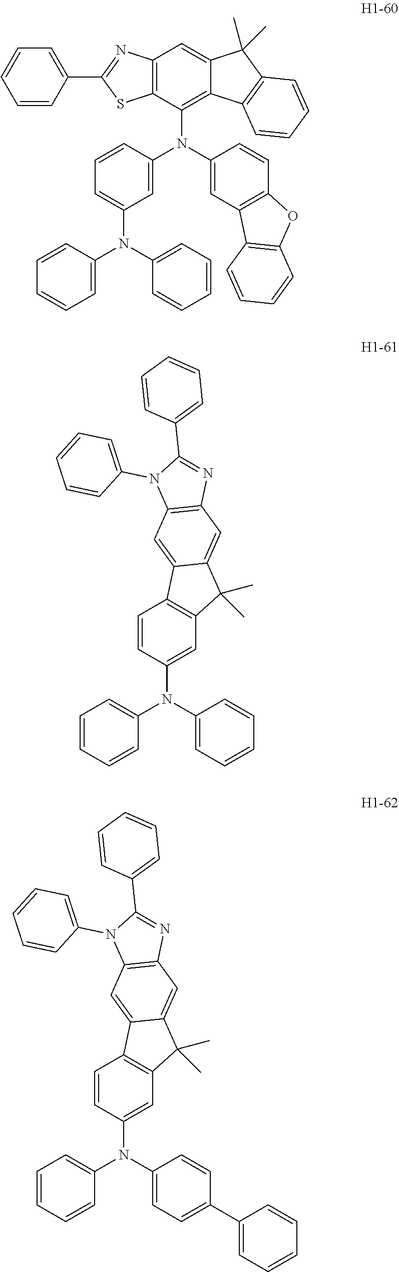 [SMILES: CC1(C)c2ccccc2-c2c1cc1nc(-c3ccccc3)sc1c2N(c1cccc(N(c2ccccc2)c2ccccc2)c1)c1ccc2oc3ccccc3c2c1 ; CC1(C)c2cc(N(c3ccccc3)c3ccccc3)ccc2-c2cc3c(cc21)nc(-c1ccccc1)n3-c1ccccc1 ; CC1(C)c2cc(N(c3ccccc3)c3ccc(-c4ccccc4)cc3)ccc2-c2cc3c(cc21)nc(-c1ccccc1)n3-c1ccccc1]

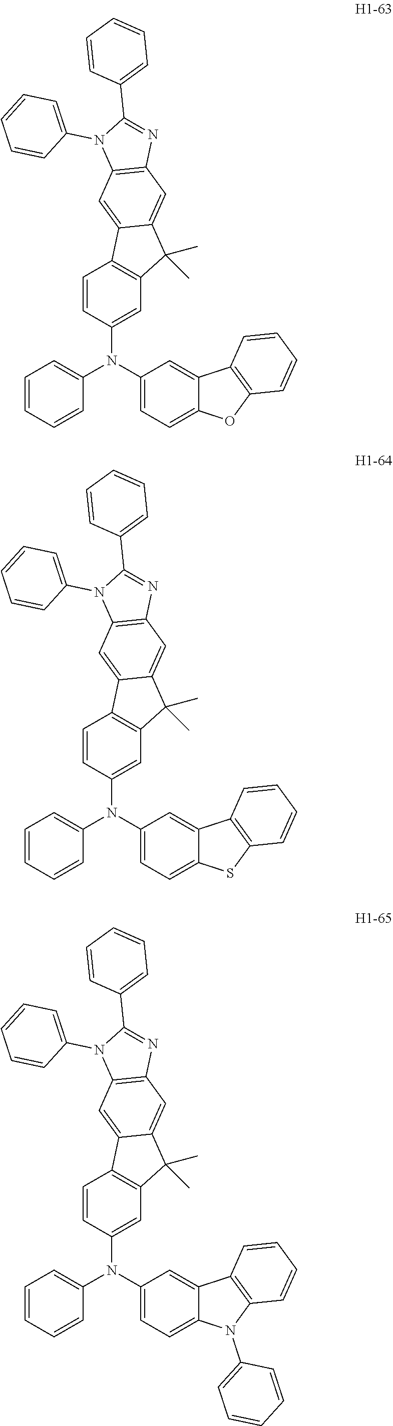 [SMILES: CC1(C)c2cc(N(c3ccccc3)c3ccc4oc5ccccc5c4c3)ccc2-c2cc3c(cc21)nc(-c1ccccc1)n3-c1ccccc1 ; CC1(C)c2cc(N(c3ccccc3)c3ccc4sc5ccccc5c4c3)ccc2-c2cc3c(cc21)nc(-c1ccccc1)n3-c1ccccc1 ; CC1(C)c2cc(N(c3ccccc3)c3ccc4c(c3)c3ccccc3n4-c3ccccc3)ccc2-c2cc3c(cc21)nc(-c1ccccc1)n3-c1ccccc1]

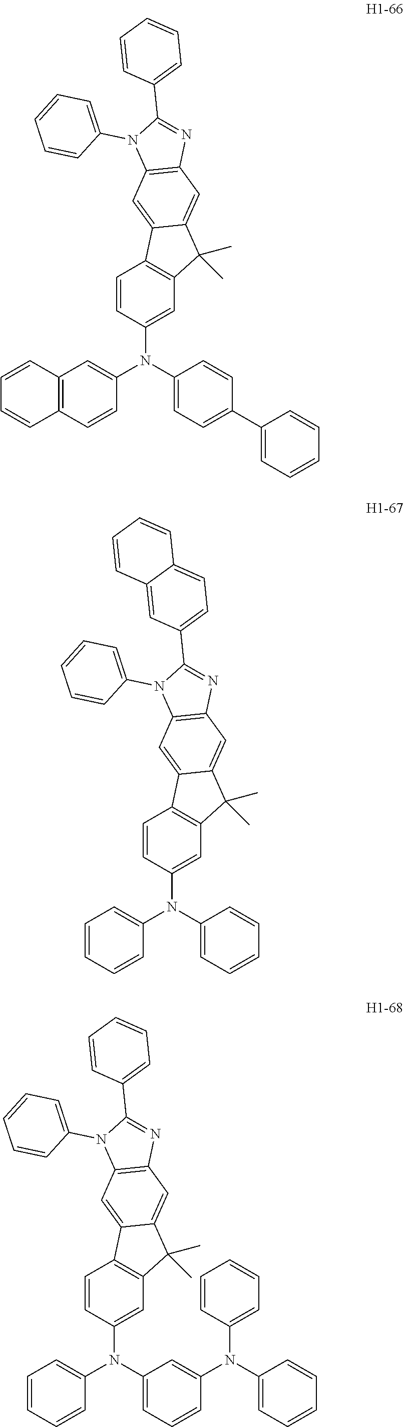

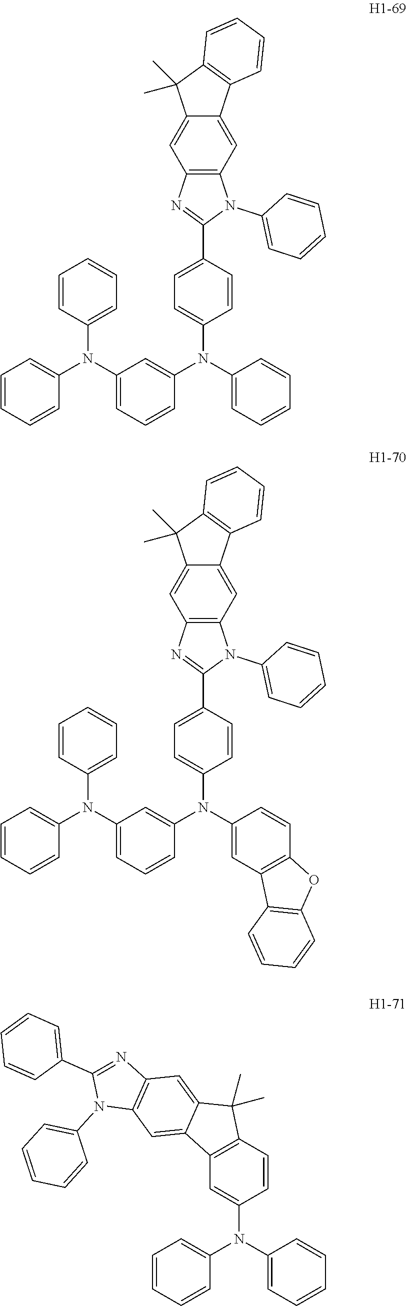 [SMILES: CC1(C)c2ccccc2-c2cc3c(cc21)nc(-c1ccc(N(c2ccccc2)c2cccc(N(c4ccccc4)c4ccccc4)c2)cc1)n3-c1ccccc1 ; CC1(C)c2ccccc2-c2cc3c(cc21)nc(-c1ccc(N(c2cccc(N(c4ccccc4)c4ccccc4)c2)c2ccc4oc5ccccc5c4c2)cc1)n3-c1ccccc1 ; CC1(C)c2ccc(N(c3ccccc3)c3ccccc3)cc2-c2cc3c(cc21)nc(-c1ccccc1)n3-c1ccccc1]

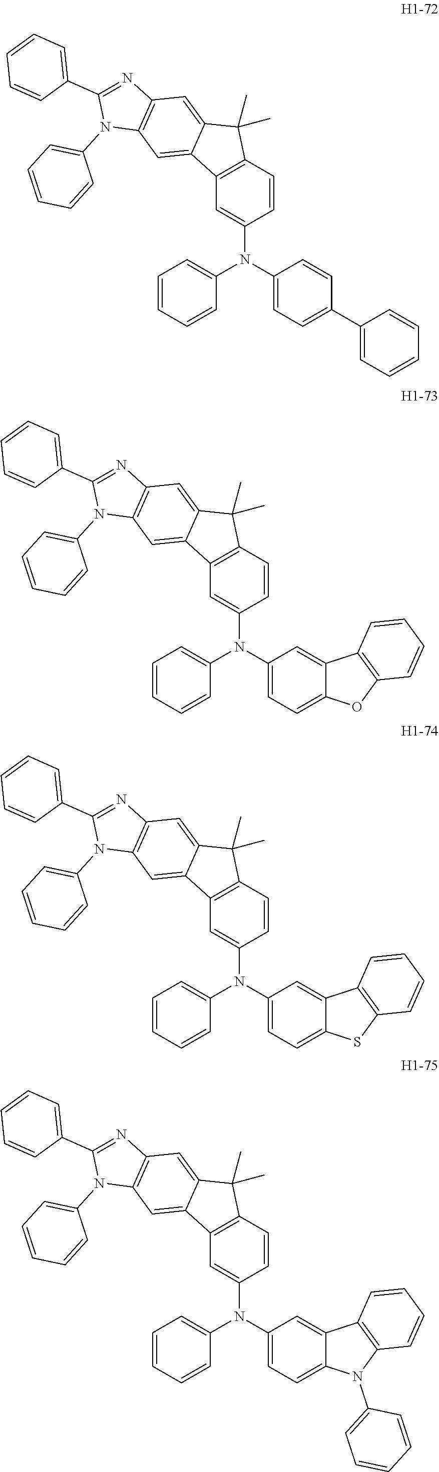 [SMILES: CC1(C)c2ccc(N(c3ccccc3)c3ccc(-c4ccccc4)cc3)cc2-c2cc3c(cc21)nc(-c1ccccc1)n3-c1ccccc1 ; CC1(C)c2ccc(N(c3ccccc3)c3ccc4oc5ccccc5c4c3)cc2-c2cc3c(cc21)nc(-c1ccccc1)n3-c1ccccc1 ; CC1(C)c2ccc(N(c3ccccc3)c3ccc4sc5ccccc5c4c3)cc2-c2cc3c(cc21)nc(-c1ccccc1)n3-c1ccccc1 ; CC1(C)c2ccc(N(c3ccccc3)c3ccc4c(c3)c3ccccc3n4-c3ccccc3)cc2-c2cc3c(cc21)nc(-c1ccccc1)n3-c1ccccc1]

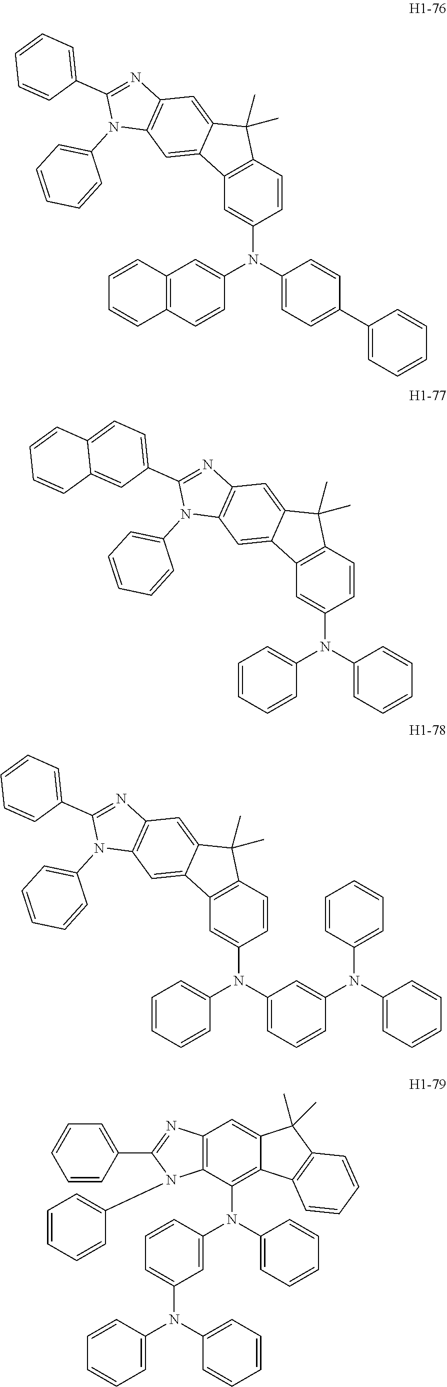 [SMILES: CC1(C)c2ccc(N(c3ccc(-c4ccccc4)cc3)c3ccc4ccccc4c3)cc2-c2cc3c(cc21)nc(-c1ccccc1)n3-c1ccccc1 ; CC1(C)c2ccc(N(c3ccccc3)c3ccccc3)cc2-c2cc3c(cc21)nc(-c1ccc2ccccc2c1)n3-c1ccccc1 ; CC1(C)c2ccc(N(c3ccccc3)c3cccc(N(c4ccccc4)c4ccccc4)c3)cc2-c2cc3c(cc21)nc(-c1ccccc1)n3-c1ccccc1 ; CC1(C)c2ccccc2-c2c1cc1nc(-c3ccccc3)n(-c3ccccc3)c1c2N(c1ccccc1)c1cccc(N(c2ccccc2)c2ccccc2)c1]

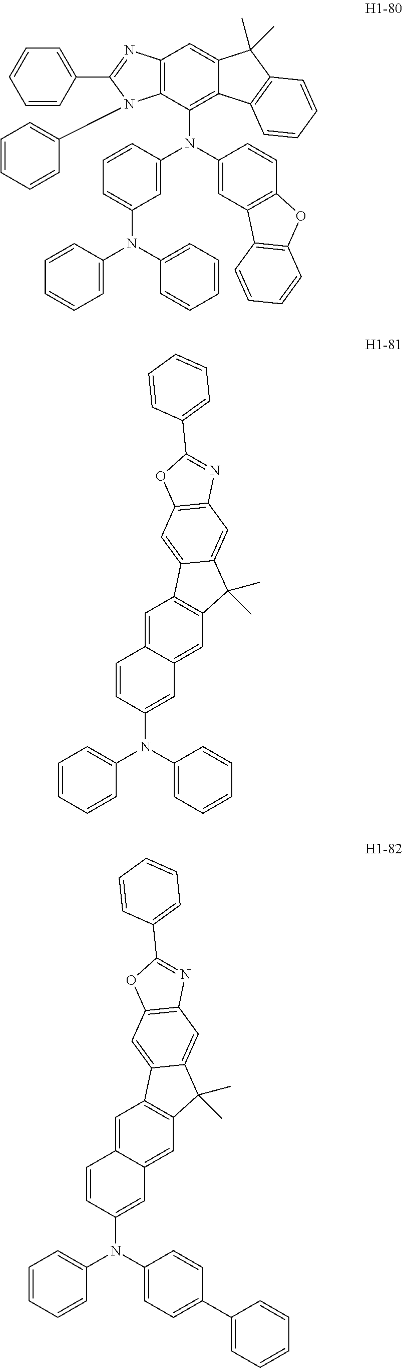

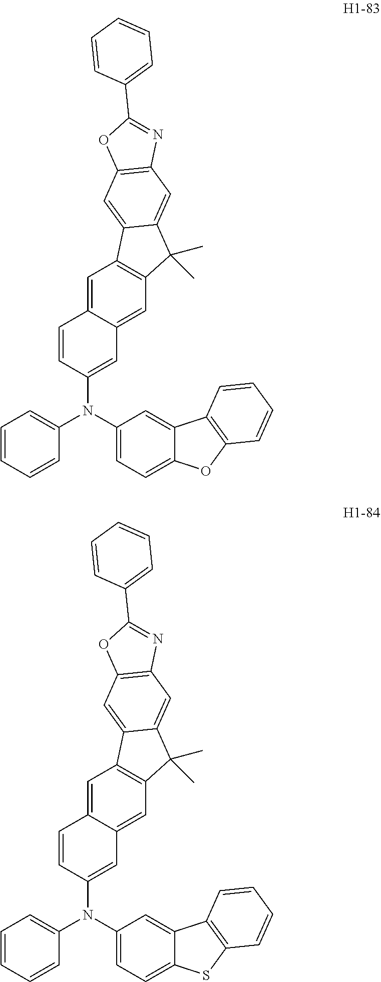 [SMILES: CC1(C)c2cc3cc(N(c4ccccc4)c4ccc5oc6ccccc6c5c4)ccc3cc2-c2cc3oc(-c4ccccc4)nc3cc21 ; CC1(C)c2cc3cc(N(c4ccccc4)c4ccc5sc6ccccc6c5c4)ccc3cc2-c2cc3oc(-c4ccccc4)nc3cc21]

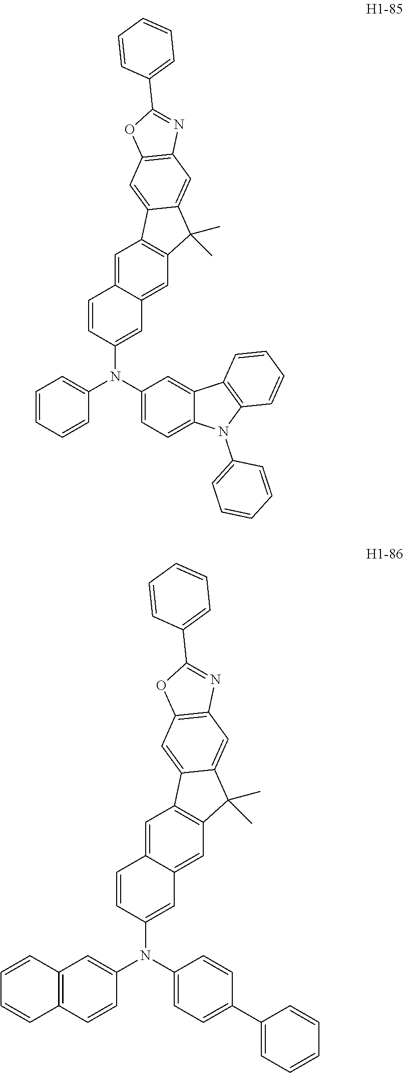 [SMILES: CC1(C)c2cc3cc(N(c4ccccc4)c4ccc5c(c4)c4ccccc4n5-c4ccccc4)ccc3cc2-c2cc3oc(-c4ccccc4)nc3cc21 ; CC1(C)c2cc3cc(N(c4ccc(-c5ccccc5)cc4)c4ccc5ccccc5c4)ccc3cc2-c2cc3oc(-c4ccccc4)nc3cc21]

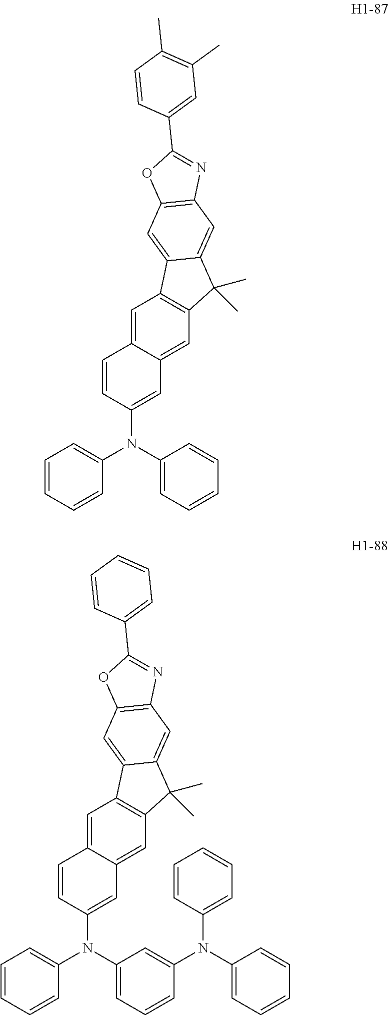 [SMILES: Cc1ccc(-c2nc3cc4c(cc3o2)-c2cc3ccc(N(c5ccccc5)c5ccccc5)cc3cc2C4(C)C)cc1C ; CC1(C)c2cc3cc(N(c4ccccc4)c4cccc(N(c5ccccc5)c5ccccc5)c4)ccc3cc2-c2cc3oc(-c4ccccc4)nc3cc21]

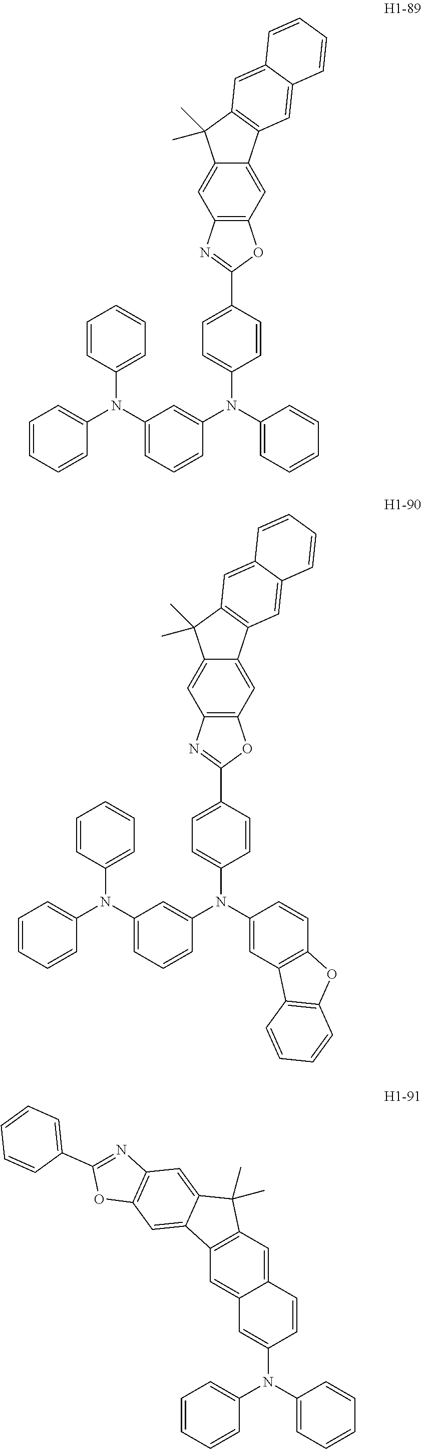

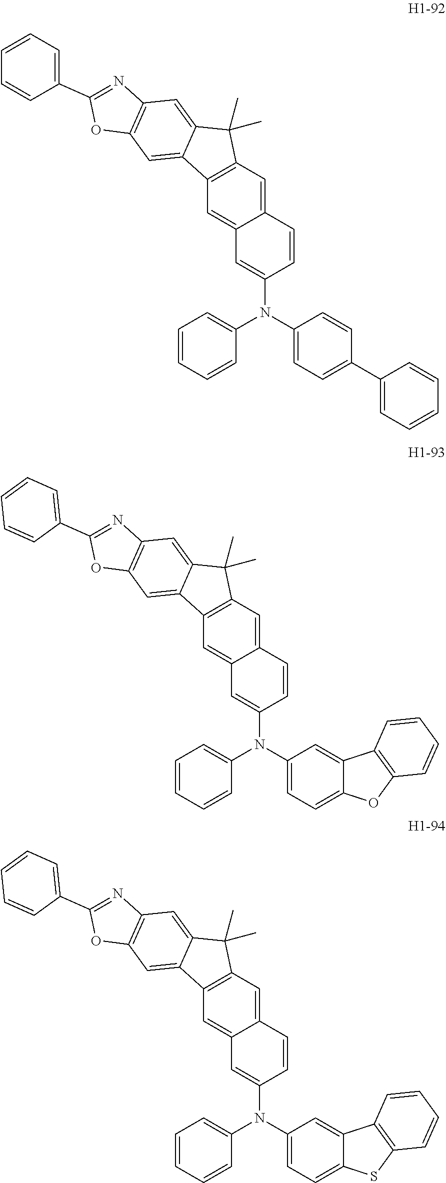 [SMILES: CC1(C)c2cc3ccc(N(c4ccccc4)c4ccc(-c5ccccc5)cc4)cc3cc2-c2cc3oc(-c4ccccc4)nc3cc21 ; CC1(C)c2cc3ccc(N(c4ccccc4)c4ccc5oc6ccccc6c5c4)cc3cc2-c2cc3oc(-c4ccccc4)nc3cc21 ; CC1(C)c2cc3ccc(N(c4ccccc4)c4ccc5sc6ccccc6c5c4)cc3cc2-c2cc3oc(-c4ccccc4)nc3cc21]

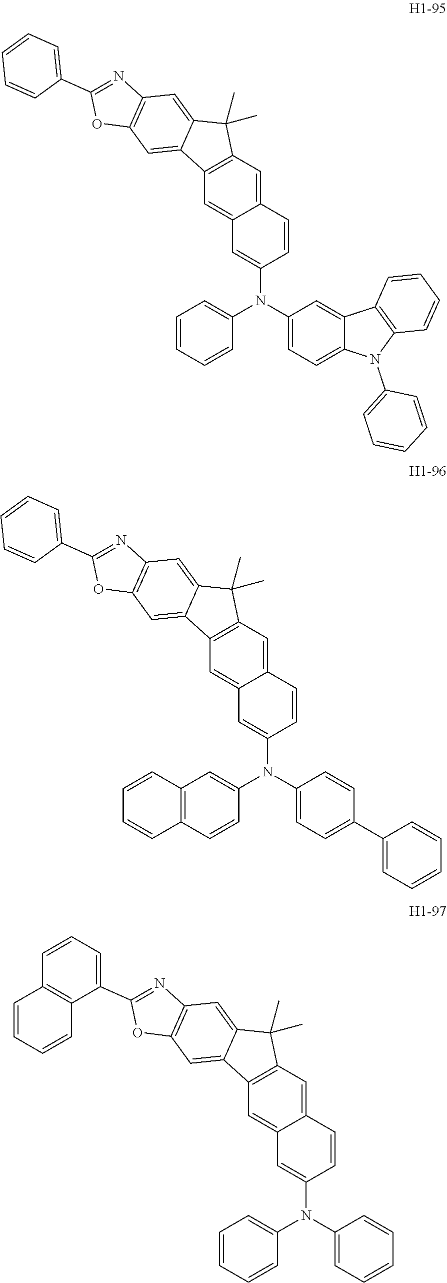 [SMILES: CC1(C)c2cc3ccc(N(c4ccccc4)c4ccc5c(c4)c4ccccc4n5-c4ccccc4)cc3cc2-c2cc3oc(-c4ccccc4)nc3cc21 ; CC1(C)c2cc3ccc(N(c4ccc(-c5ccccc5)cc4)c4ccc5ccccc5c4)cc3cc2-c2cc3oc(-c4ccccc4)nc3cc21 ; CC1(C)c2cc3ccc(N(c4ccccc4)c4ccccc4)cc3cc2-c2cc3oc(-c4cccc5ccccc45)nc3cc21]

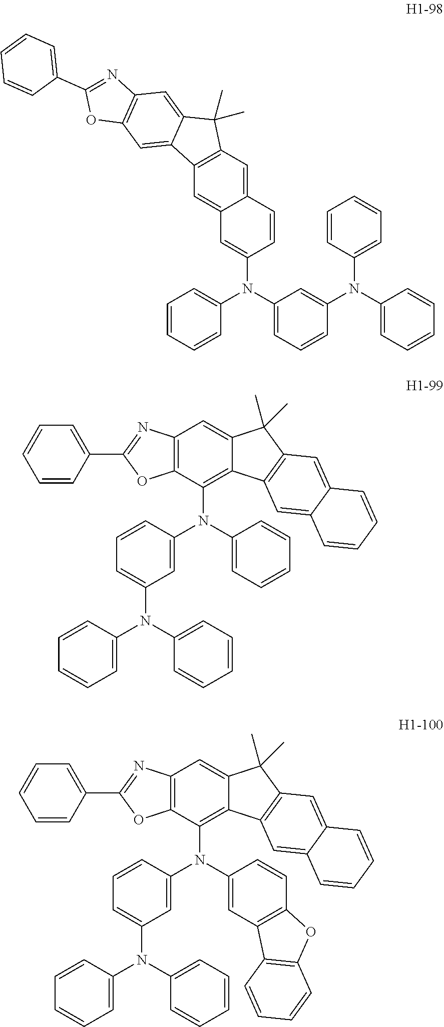 [SMILES: CC1(C)c2cc3ccc(N(c4ccccc4)c4cccc(N(c5ccccc5)c5ccccc5)c4)cc3cc2-c2cc3oc(-c4ccccc4)nc3cc21 ; CC1(C)c2cc3ccccc3cc2-c2c1cc1nc(-c3ccccc3)oc1c2N(c1ccccc1)c1cccc(N(c2ccccc2)c2ccccc2)c1 ; CC1(C)c2cc3ccccc3cc2-c2c1cc1nc(-c3ccccc3)oc1c2N(c1cccc(N(c2ccccc2)c2ccccc2)c1)c1ccc2oc3ccccc3c2c1]

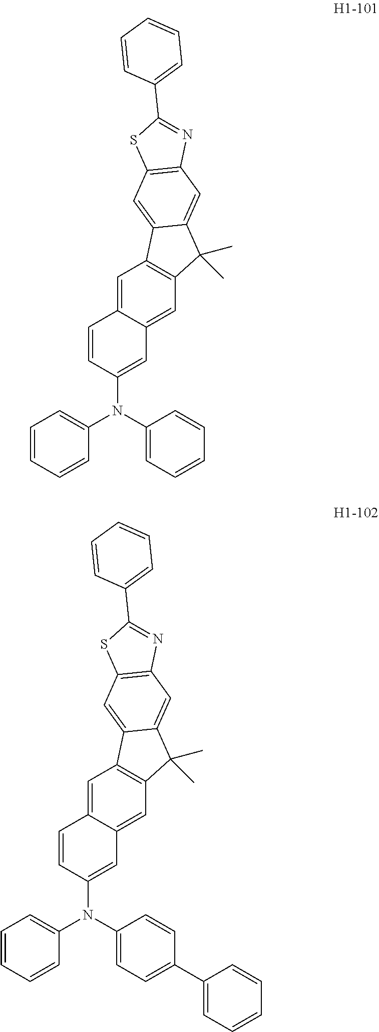 [SMILES: CC1(C)c2cc3cc(N(c4ccccc4)c4ccccc4)ccc3cc2-c2cc3sc(-c4ccccc4)nc3cc21 ; CC1(C)c2cc3cc(N(c4ccccc4)c4ccc(-c5ccccc5)cc4)ccc3cc2-c2cc3sc(-c4ccccc4)nc3cc21]

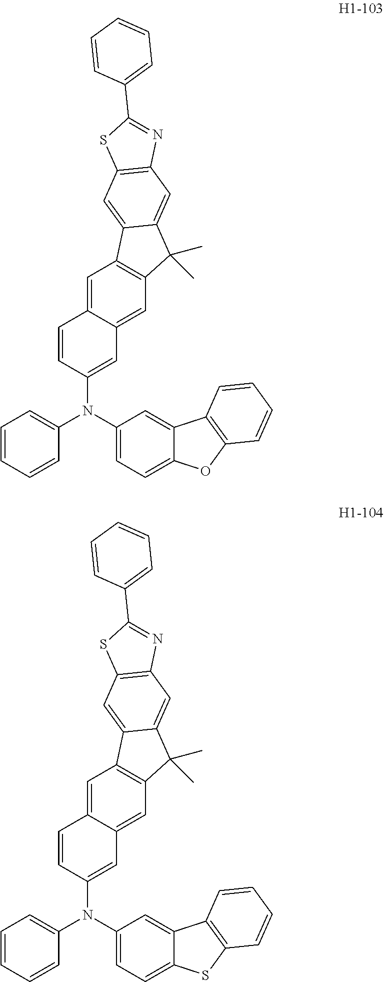 [SMILES: CC1(C)c2cc3cc(N(c4ccccc4)c4ccc5oc6ccccc6c5c4)ccc3cc2-c2cc3sc(-c4ccccc4)nc3cc21 ; CC1(C)c2cc3cc(N(c4ccccc4)c4ccc5sc6ccccc6c5c4)ccc3cc2-c2cc3sc(-c4ccccc4)nc3cc21]

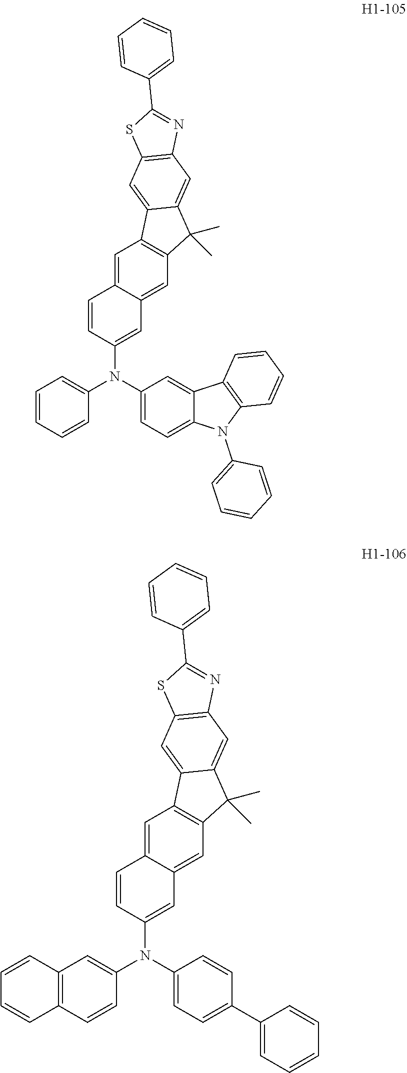 [SMILES: CC1(C)c2cc3cc(N(c4ccccc4)c4ccc5c(c4)c4ccccc4n5-c4ccccc4)ccc3cc2-c2cc3sc(-c4ccccc4)nc3cc21 ; CC1(C)c2cc3cc(N(c4ccc(-c5ccccc5)cc4)c4ccc5ccccc5c4)ccc3cc2-c2cc3sc(-c4ccccc4)nc3cc21]

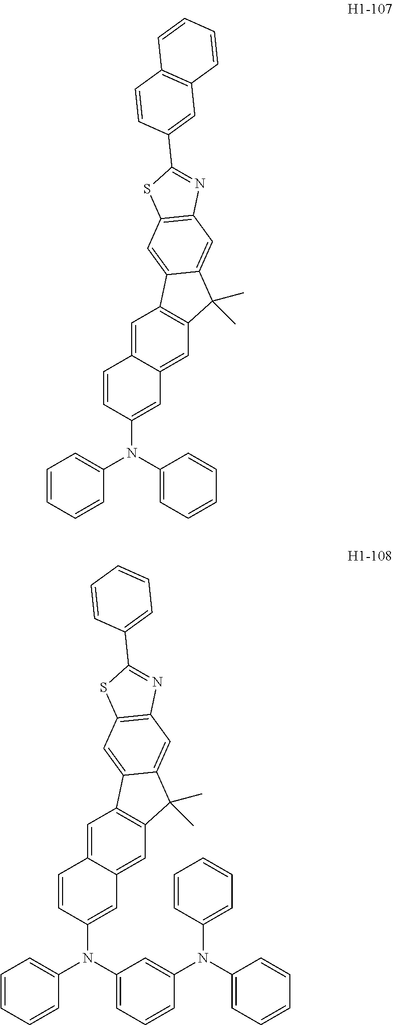 [SMILES: CC1(C)c2cc3cc(N(c4ccccc4)c4ccccc4)ccc3cc2-c2cc3sc(-c4ccc5ccccc5c4)nc3cc21 ; CC1(C)c2cc3cc(N(c4ccccc4)c4cccc(N(c5ccccc5)c5ccccc5)c4)ccc3cc2-c2cc3sc(-c4ccccc4)nc3cc21]

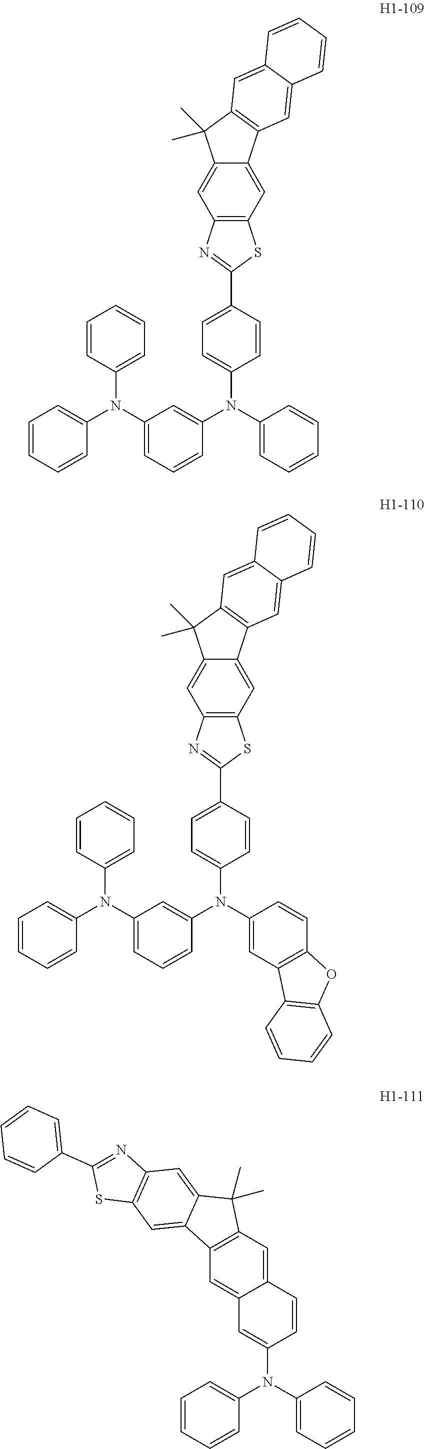

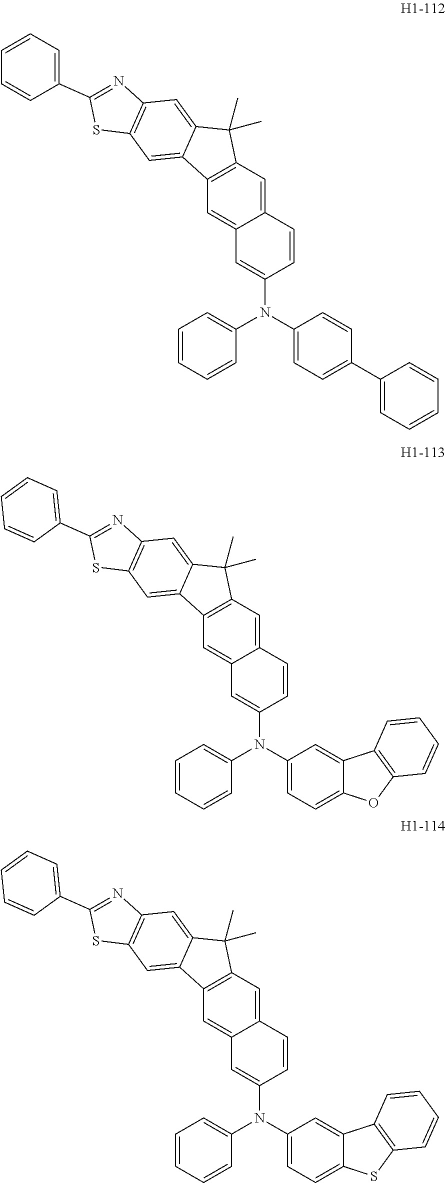 [SMILES: CC1(C)c2cc3ccc(N(c4ccccc4)c4ccc(-c5ccccc5)cc4)cc3cc2-c2cc3sc(-c4ccccc4)nc3cc21 ; CC1(C)c2cc3ccc(N(c4ccccc4)c4ccc5oc6ccccc6c5c4)cc3cc2-c2cc3sc(-c4ccccc4)nc3cc21 ; CC1(C)c2cc3ccc(N(c4ccccc4)c4ccc5sc6ccccc6c5c4)cc3cc2-c2cc3sc(-c4ccccc4)nc3cc21]

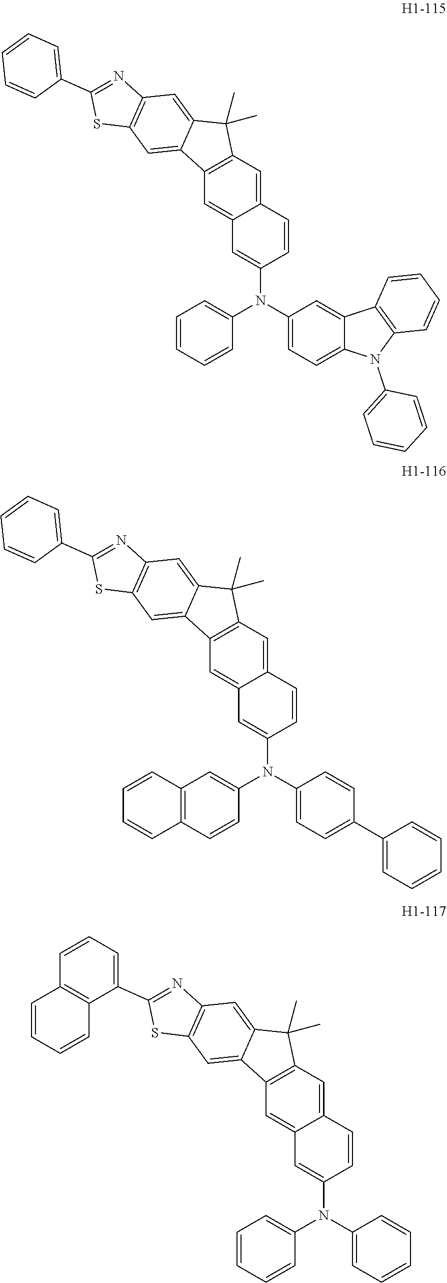 [SMILES: CC1(C)c2cc3ccc(N(c4ccccc4)c4ccc5c(c4)c4ccccc4n5-c4ccccc4)cc3cc2-c2cc3sc(-c4ccccc4)nc3cc21 ; CC1(C)c2cc3ccc(N(c4ccc(-c5ccccc5)cc4)c4ccc5ccccc5c4)cc3cc2-c2cc3sc(-c4ccccc4)nc3cc21 ; CC1(C)c2cc3ccc(N(c4ccccc4)c4ccccc4)cc3cc2-c2cc3sc(-c4cccc5ccccc45)nc3cc21]

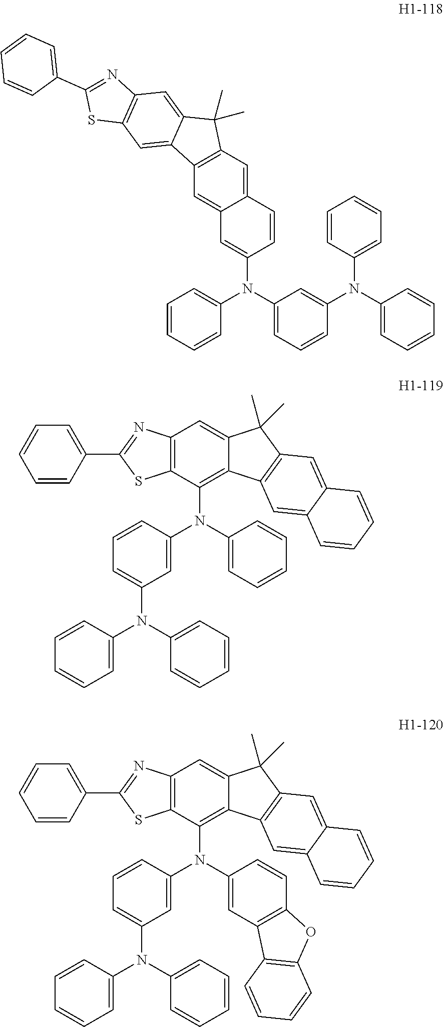 [SMILES: CC1(C)c2cc3ccc(N(c4ccccc4)c4cccc(N(c5ccccc5)c5ccccc5)c4)cc3cc2-c2cc3sc(-c4ccccc4)nc3cc21 ; CC1(C)c2cc3ccccc3cc2-c2c1cc1nc(-c3ccccc3)sc1c2N(c1ccccc1)c1cccc(N(c2ccccc2)c2ccccc2)c1 ; CC1(C)c2cc3ccccc3cc2-c2c1cc1nc(-c3ccccc3)sc1c2N(c1cccc(N(c2ccccc2)c2ccccc2)c1)c1ccc2oc3ccccc3c2c1]

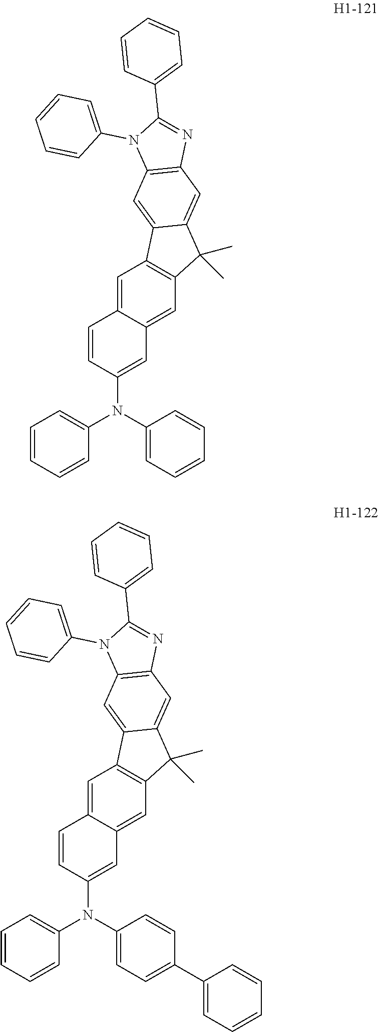 [SMILES: CC1(C)c2cc3cc(N(c4ccccc4)c4ccccc4)ccc3cc2-c2cc3c(cc21)nc(-c1ccccc1)n3-c1ccccc1 ; CC1(C)c2cc3cc(N(c4ccccc4)c4ccc(-c5ccccc5)cc4)ccc3cc2-c2cc3c(cc21)nc(-c1ccccc1)n3-c1ccccc1]

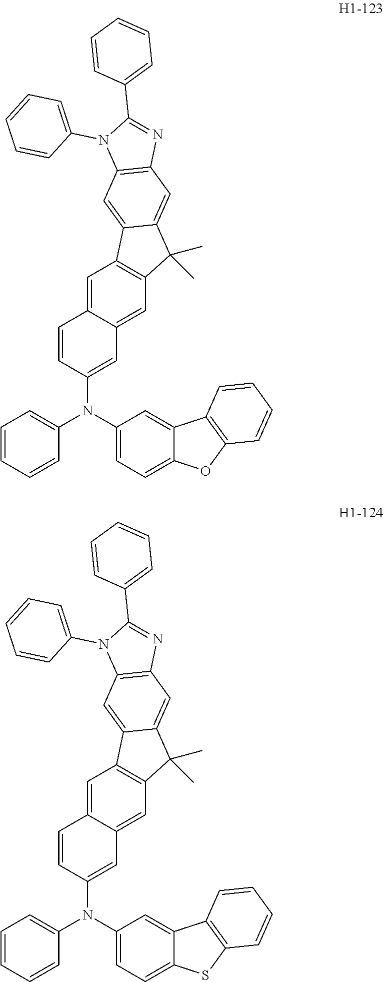 [SMILES: CC1(C)c2cc3cc(N(c4ccccc4)c4ccc5oc6ccccc6c5c4)ccc3cc2-c2cc3c(cc21)nc(-c1ccccc1)n3-c1ccccc1 ; CC1(C)c2cc3cc(N(c4ccccc4)c4ccc5sc6ccccc6c5c4)ccc3cc2-c2cc3c(cc21)nc(-c1ccccc1)n3-c1ccccc1]

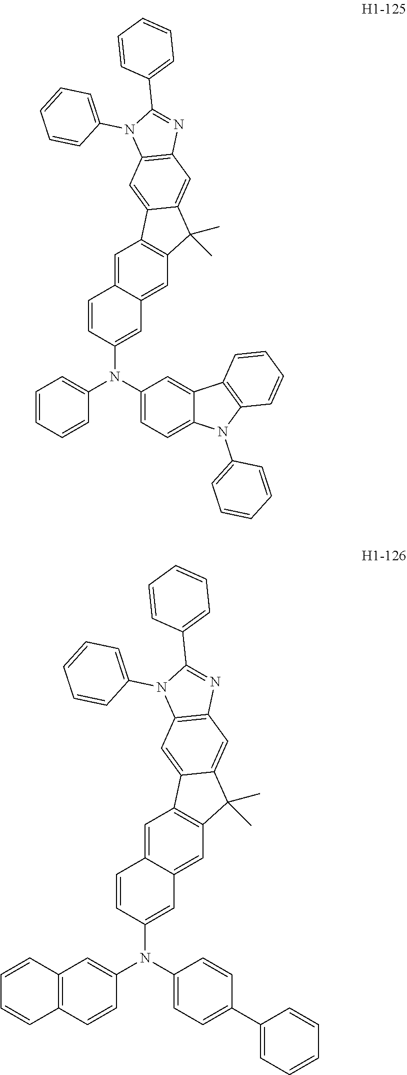 [SMILES: CC1(C)c2cc3cc(N(c4ccccc4)c4ccc5c(c4)c4ccccc4n5-c4ccccc4)ccc3cc2-c2cc3c(cc21)nc(-c1ccccc1)n3-c1ccccc1 ; CC1(C)c2cc3cc(N(c4ccc(-c5ccccc5)cc4)c4ccc5ccccc5c4)ccc3cc2-c2cc3c(cc21)nc(-c1ccccc1)n3-c1ccccc1]

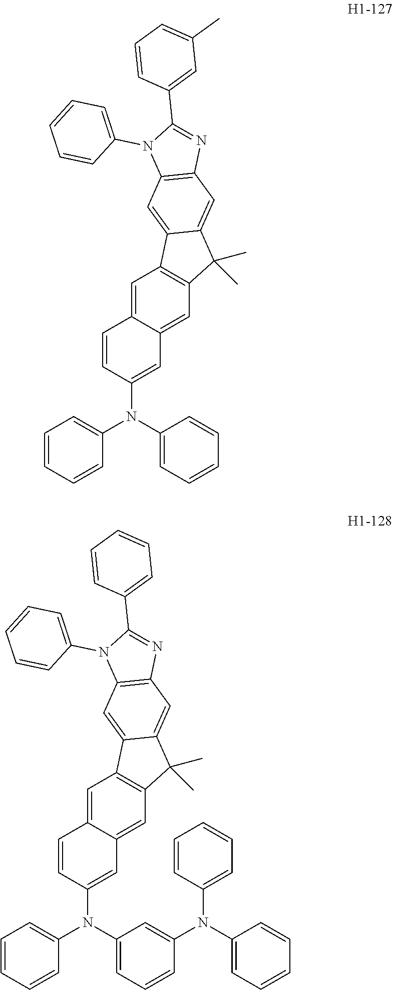 [SMILES: Cc1cccc(-c2nc3cc4c(cc3n2-c2ccccc2)-c2cc3ccc(N(c5ccccc5)c5ccccc5)cc3cc2C4(C)C)c1 ; CC1(C)c2cc3cc(N(c4ccccc4)c4cccc(N(c5ccccc5)c5ccccc5)c4)ccc3cc2-c2cc3c(cc21)nc(-c1ccccc1)n3-c1ccccc1]

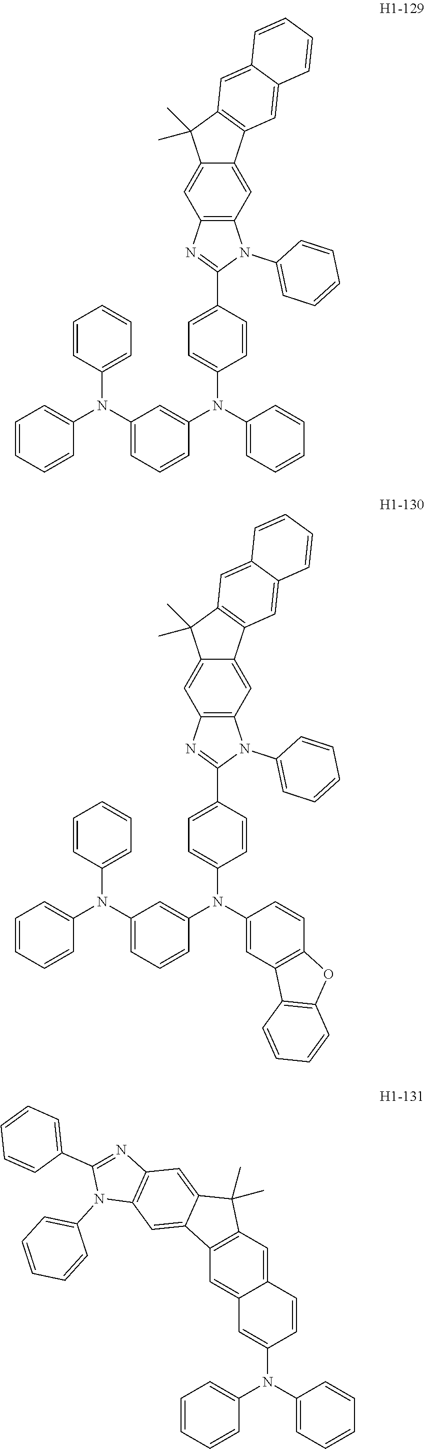

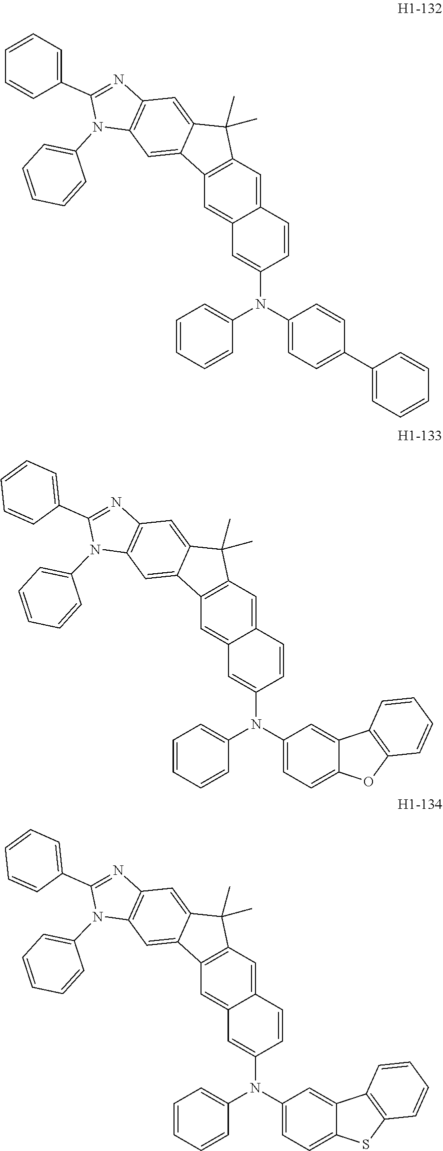 [SMILES: CC1(C)c2cc3ccc(N(c4ccccc4)c4ccc(-c5ccccc5)cc4)cc3cc2-c2cc3c(cc21)nc(-c1ccccc1)n3-c1ccccc1 ; CC1(C)c2cc3ccc(N(c4ccccc4)c4ccc5oc6ccccc6c5c4)cc3cc2-c2cc3c(cc21)nc(-c1ccccc1)n3-c1ccccc1 ; CC1(C)c2cc3ccc(N(c4ccccc4)c4ccc5sc6ccccc6c5c4)cc3cc2-c2cc3c(cc21)nc(-c1ccccc1)n3-c1ccccc1]

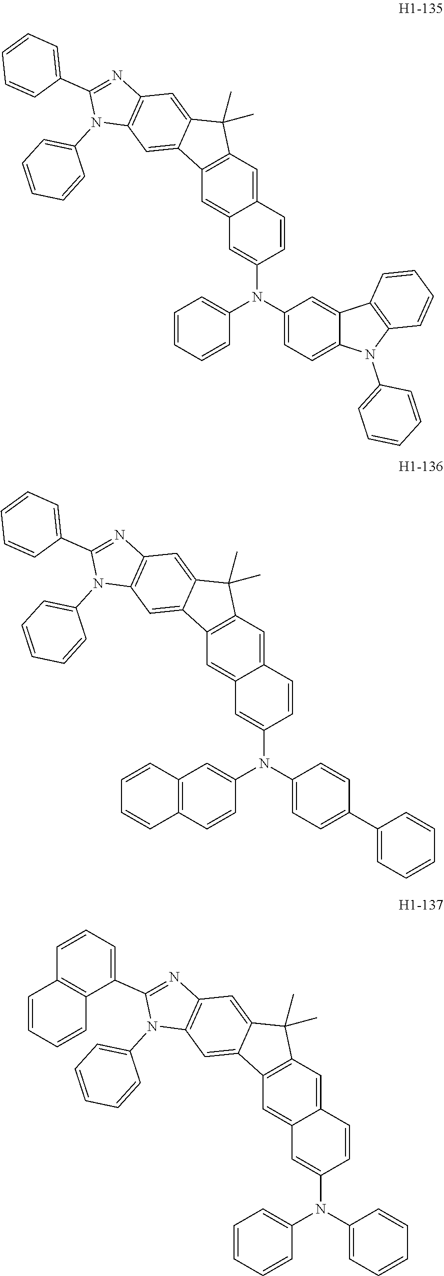 [SMILES: CC1(C)c2cc3ccc(N(c4ccccc4)c4ccc5c(c4)c4ccccc4n5-c4ccccc4)cc3cc2-c2cc3c(cc21)nc(-c1ccccc1)n3-c1ccccc1 ; CC1(C)c2cc3ccc(N(c4ccc(-c5ccccc5)cc4)c4ccc5ccccc5c4)cc3cc2-c2cc3c(cc21)nc(-c1ccccc1)n3-c1ccccc1 ; CC1(C)c2cc3ccc(N(c4ccccc4)c4ccccc4)cc3cc2-c2cc3c(cc21)nc(-c1cccc2ccccc12)n3-c1ccccc1]

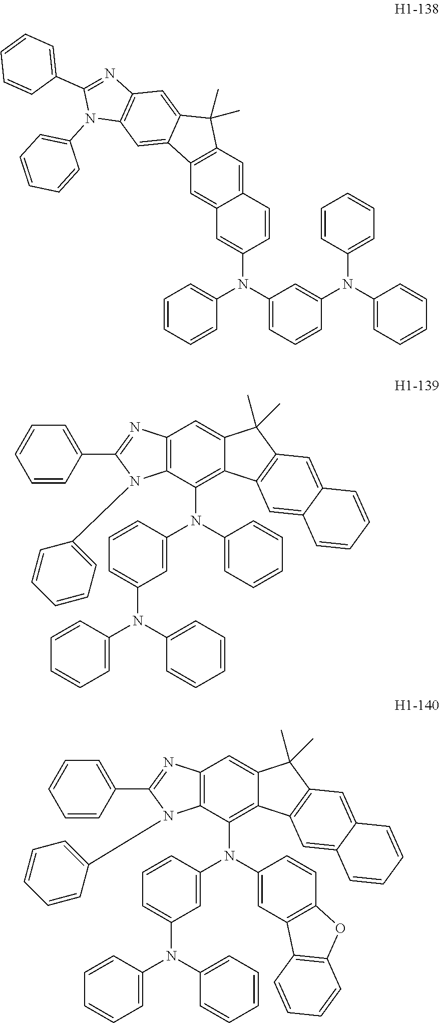 [SMILES: CC1(C)c2cc3ccc(N(c4ccccc4)c4cccc(N(c5ccccc5)c5ccccc5)c4)cc3cc2-c2cc3c(cc21)nc(-c1ccccc1)n3-c1ccccc1 ; CC1(C)c2cc3ccccc3cc2-c2c1cc1nc(-c3ccccc3)n(-c3ccccc3)c1c2N(c1ccccc1)c1cccc(N(c2ccccc2)c2ccccc2)c1 ; CC1(C)c2cc3ccccc3cc2-c2c1cc1nc(-c3ccccc3)n(-c3ccccc3)c1c2N(c1cccc(N(c2ccccc2)c2ccccc2)c1)c1ccc2oc3ccccc3c2c1]

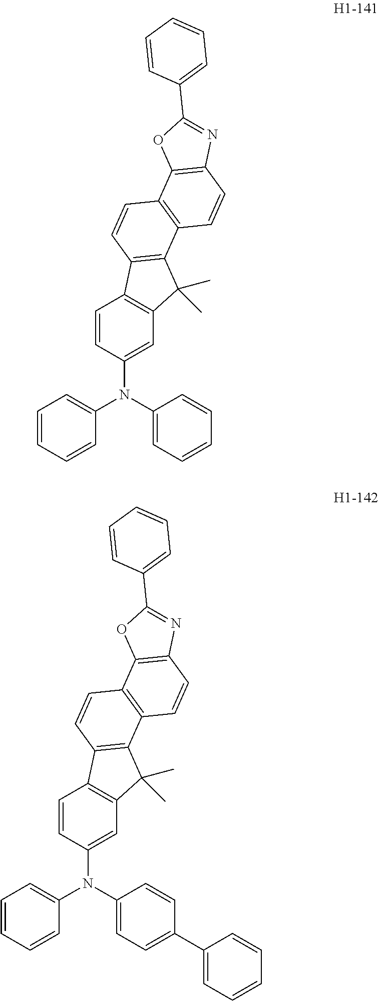 [SMILES: CC1(C)c2cc(N(c3ccccc3)c3ccccc3)ccc2-c2ccc3c(ccc4nc(-c5ccccc5)oc43)c21 ; CC1(C)c2cc(N(c3ccccc3)c3ccc(-c4ccccc4)cc3)ccc2-c2ccc3c(ccc4nc(-c5ccccc5)oc43)c21]

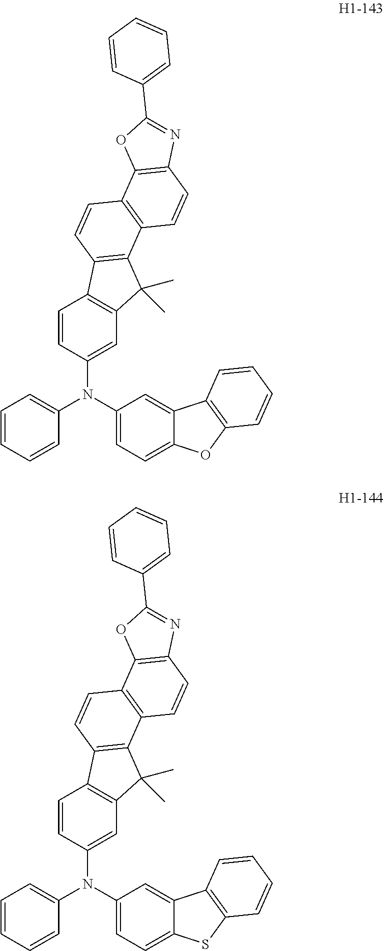 [SMILES: CC1(C)c2cc(N(c3ccccc3)c3ccc4oc5ccccc5c4c3)ccc2-c2ccc3c(ccc4nc(-c5ccccc5)oc43)c21 ; CC1(C)c2cc(N(c3ccccc3)c3ccc4sc5ccccc5c4c3)ccc2-c2ccc3c(ccc4nc(-c5ccccc5)oc43)c21]

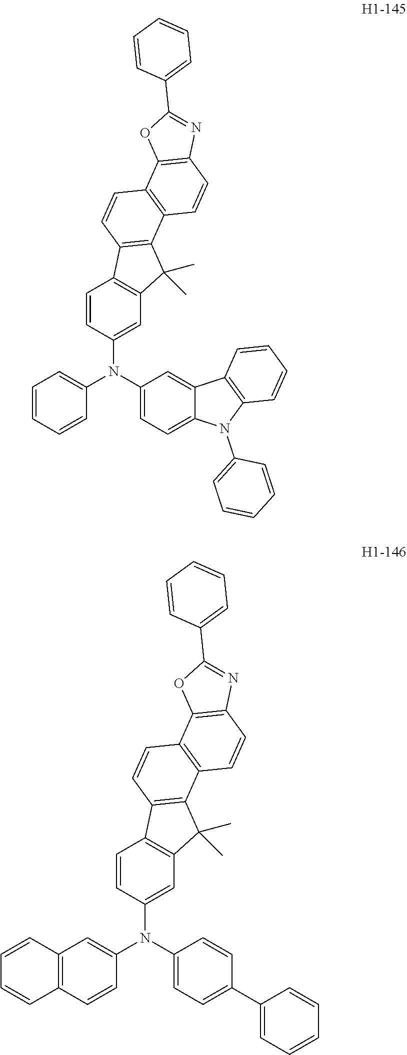 [SMILES: CC1(C)c2cc(N(c3ccccc3)c3ccc4c(c3)c3ccccc3n4-c3ccccc3)ccc2-c2ccc3c(ccc4nc(-c5ccccc5)oc43)c21 ; CC1(C)c2cc(N(c3ccc(-c4ccccc4)cc3)c3ccc4ccccc4c3)ccc2-c2ccc3c(ccc4nc(-c5ccccc5)oc43)c21]

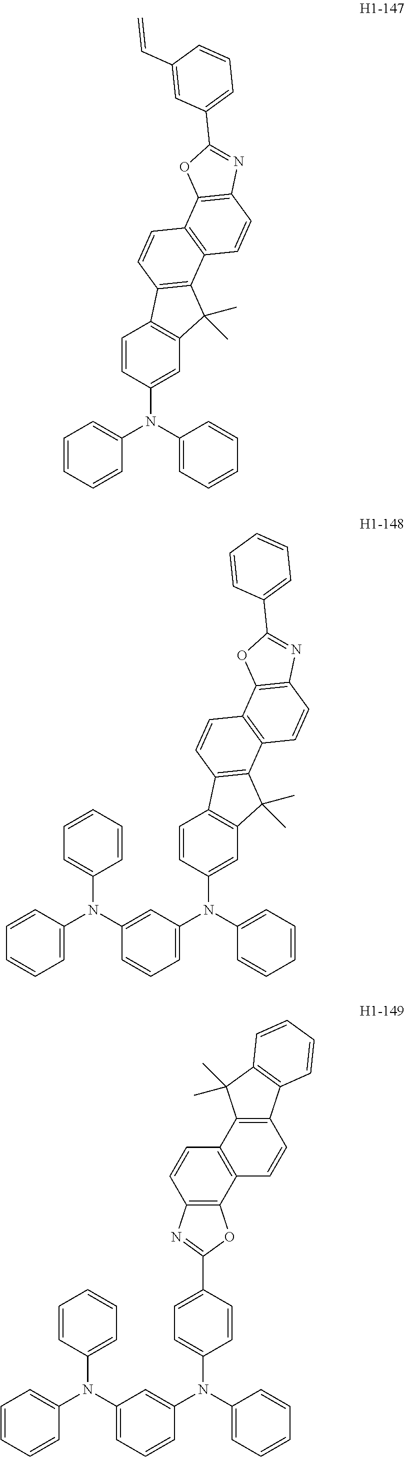 [SMILES: C=Cc1cccc(-c2nc3ccc4c5c(ccc4c3o2)-c2ccc(N(c3ccccc3)c3ccccc3)cc2C5(C)C)c1 ; CC1(C)c2cc(N(c3ccccc3)c3cccc(N(c4ccccc4)c4ccccc4)c3)ccc2-c2ccc3c(ccc4nc(-c5ccccc5)oc43)c21 ; CC1(C)c2ccccc2-c2ccc3c(ccc4nc(-c5ccc(N(c6ccccc6)c6cccc(N(c7ccccc7)c7ccccc7)c6)cc5)oc43)c21]

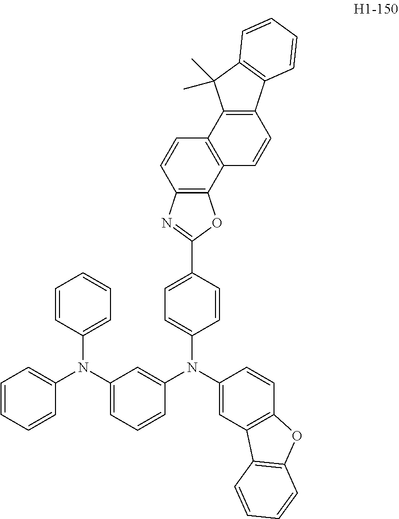 [SMILES: CC1(C)c2ccccc2-c2ccc3c(ccc4nc(-c5ccc(N(c6cccc(N(c7ccccc7)c7ccccc7)c6)c6ccc7oc8ccccc8c7c6)cc5)oc43)c21]

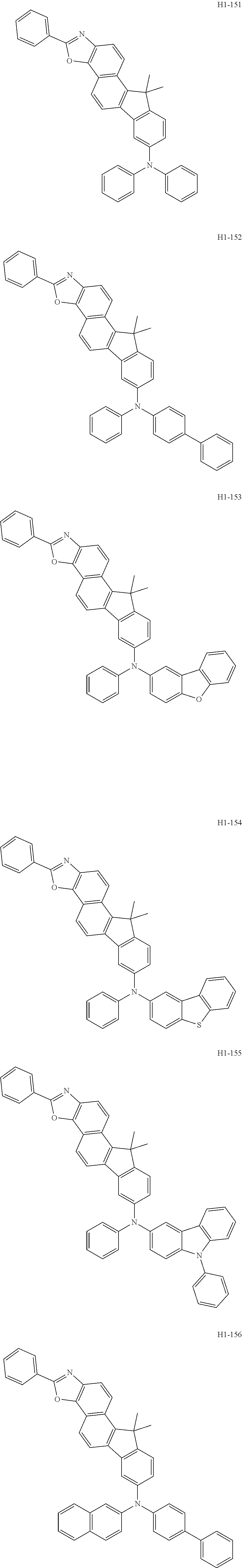

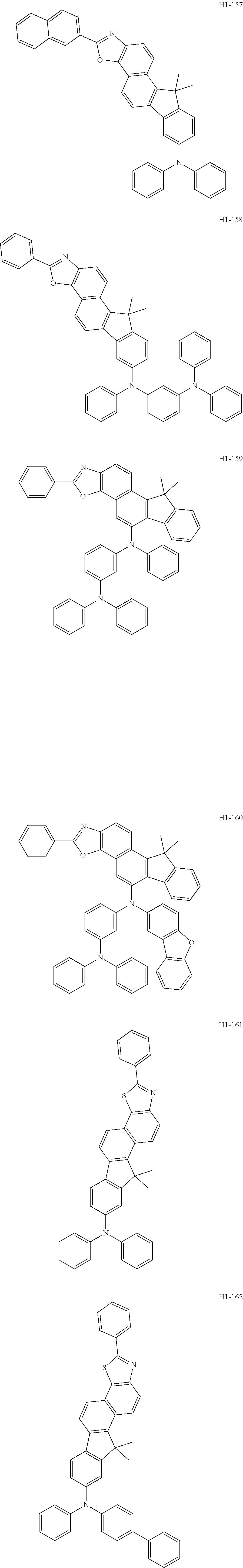

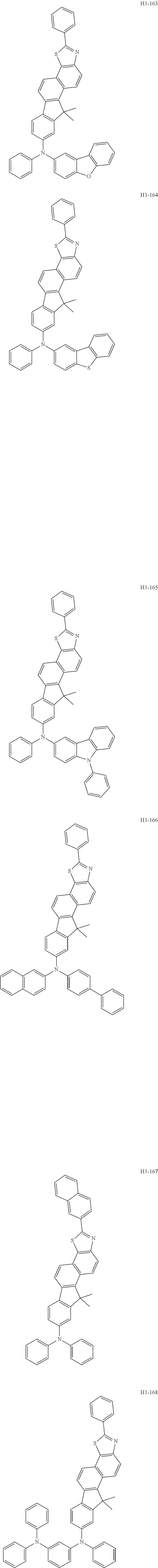

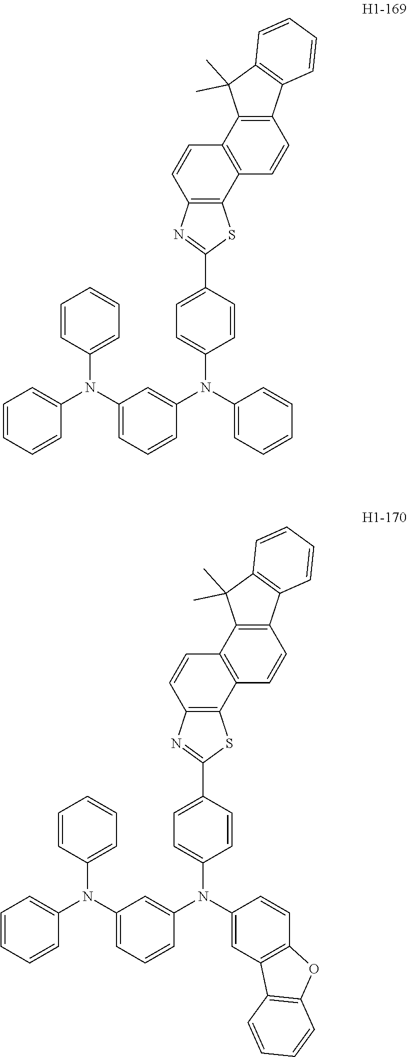 [SMILES: CC1(C)c2ccccc2-c2ccc3c(ccc4nc(-c5ccc(N(c6ccccc6)c6cccc(N(c7ccccc7)c7ccccc7)c6)cc5)sc43)c21 ; CC1(C)c2ccccc2-c2ccc3c(ccc4nc(-c5ccc(N(c6cccc(N(c7ccccc7)c7ccccc7)c6)c6ccc7oc8ccccc8c7c6)cc5)sc43)c21]

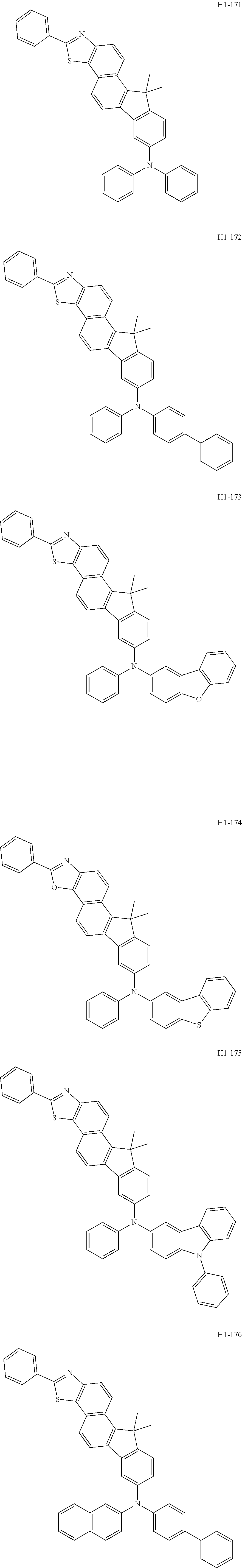

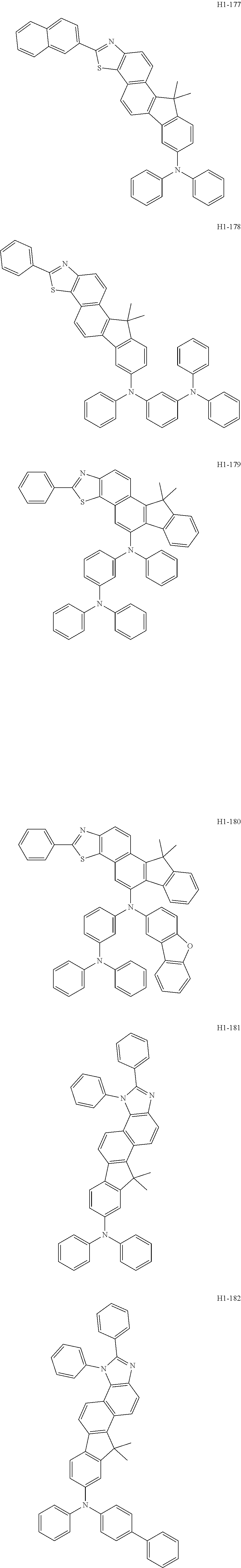

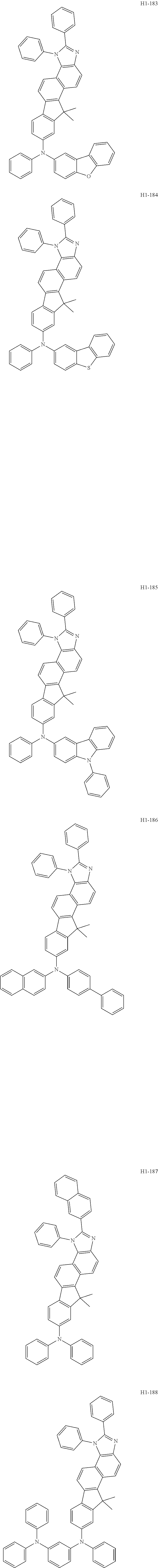

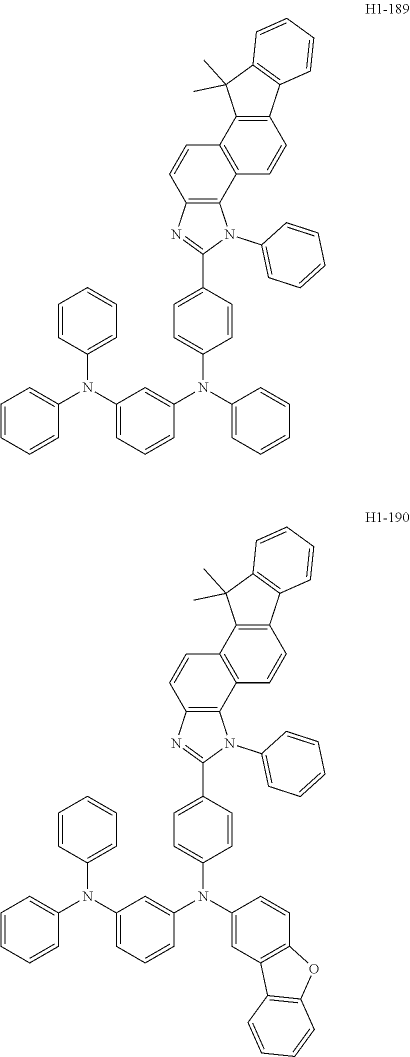 [SMILES: CC1(C)c2ccccc2-c2ccc3c(ccc4nc(-c5ccc(N(c6ccccc6)c6cccc(N(c7ccccc7)c7ccccc7)c6)cc5)n(-c5ccccc5)c43)c21 ; CC1(C)c2ccccc2-c2ccc3c(ccc4nc(-c5ccc(N(c6cccc(N(c7ccccc7)c7ccccc7)c6)c6ccc7oc8ccccc8c7c6)cc5)n(-c5ccccc5)c43)c21]

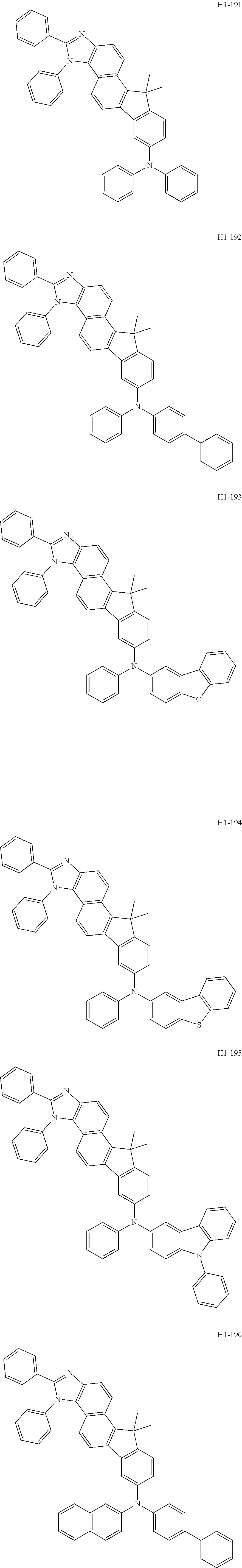

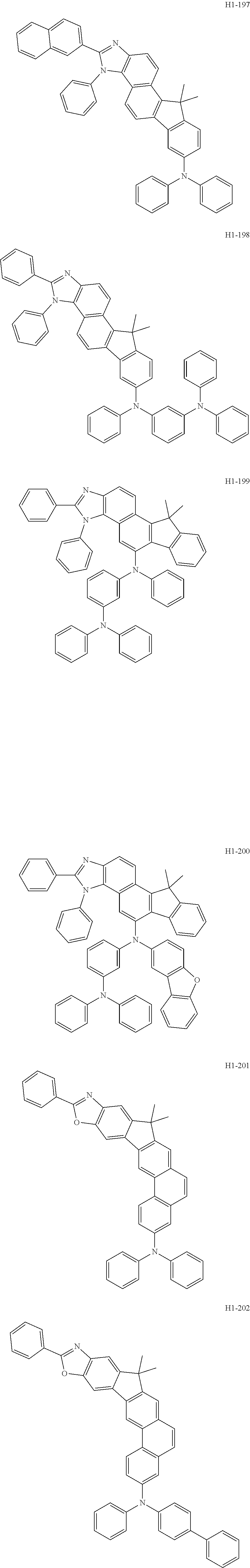

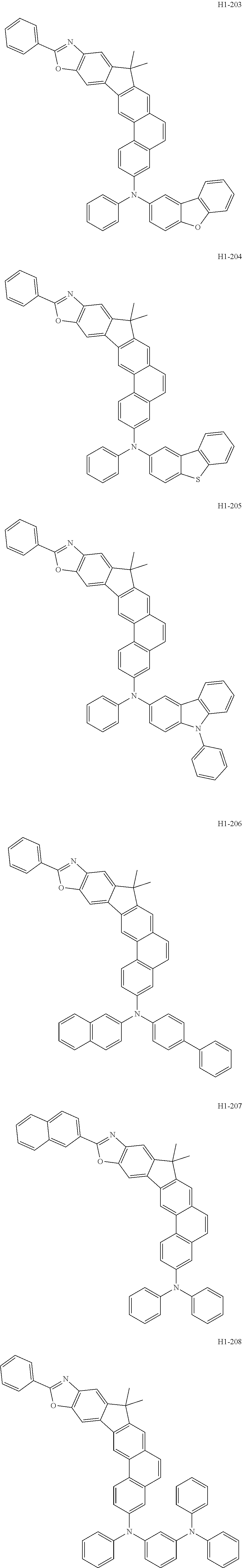

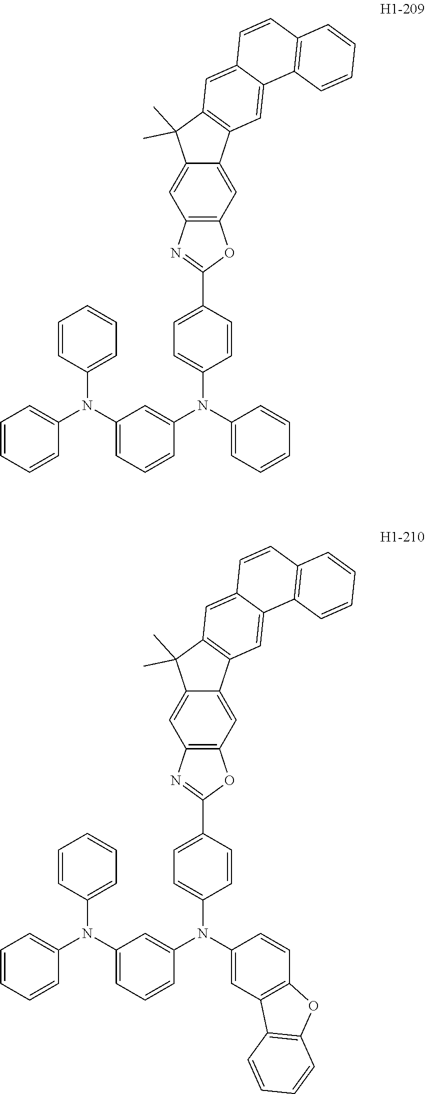 [SMILES: CC1(C)c2cc3ccc4ccccc4c3cc2-c2cc3oc(-c4ccc(N(c5ccccc5)c5cccc(N(c6ccccc6)c6ccccc6)c5)cc4)nc3cc21 ; CC1(C)c2cc3ccc4ccccc4c3cc2-c2cc3oc(-c4ccc(N(c5cccc(N(c6ccccc6)c6ccccc6)c5)c5ccc6oc7ccccc7c6c5)cc4)nc3cc21]

[chemical structure]

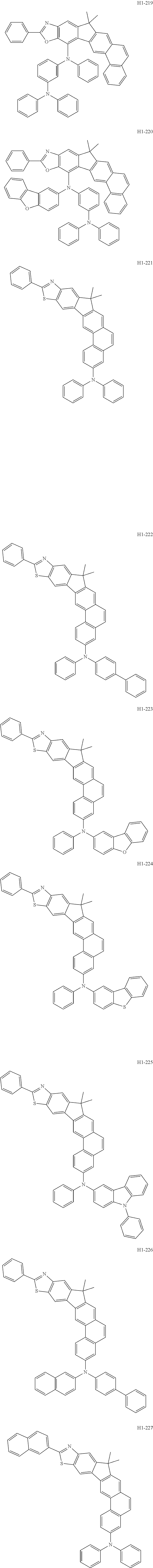

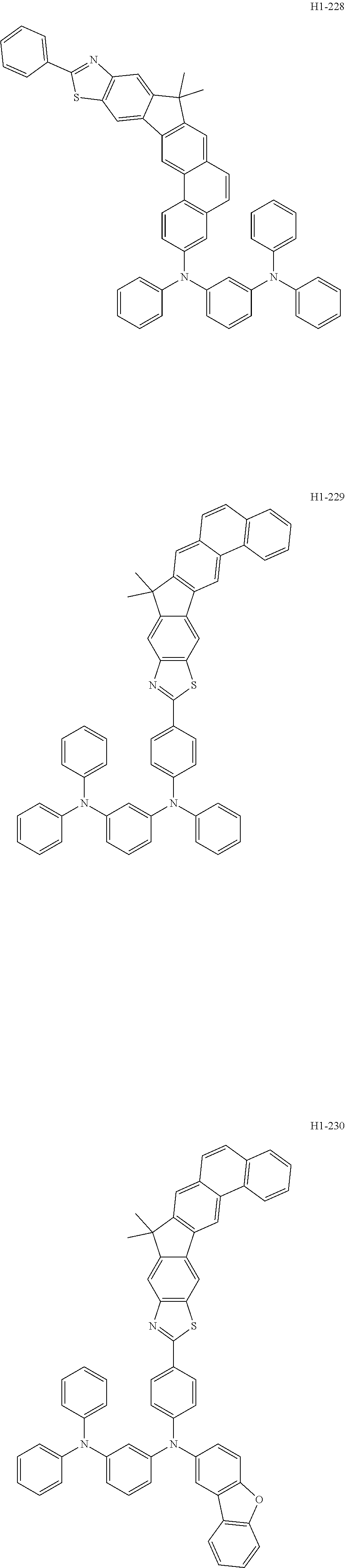 [SMILES: CC1(C)c2cc3ccc4cc(N(c5ccccc5)c5cccc(N(c6ccccc6)c6ccccc6)c5)ccc4c3cc2-c2cc3sc(-c4ccccc4)nc3cc21 ; CC1(C)c2cc3ccc4ccccc4c3cc2-c2cc3sc(-c4ccc(N(c5ccccc5)c5cccc(N(c6ccccc6)c6ccccc6)c5)cc4)nc3cc21 ; CC1(C)c2cc3ccc4ccccc4c3cc2-c2cc3sc(-c4ccc(N(c5cccc(N(c6ccccc6)c6ccccc6)c5)c5ccc6oc7ccccc7c6c5)cc4)nc3cc21]

[chemical structure]

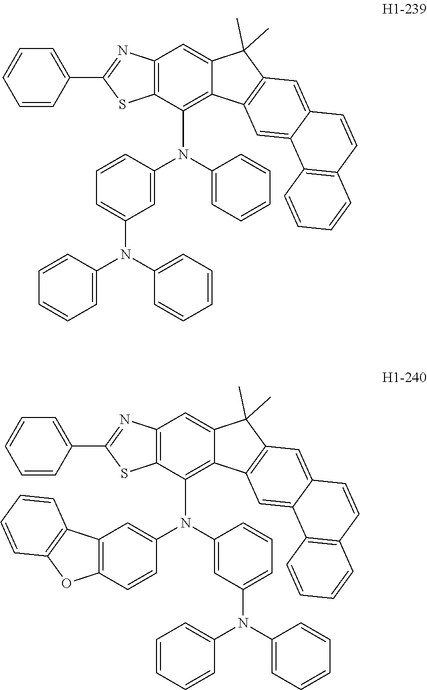 [SMILES: CC1(C)c2cc3ccc4ccccc4c3cc2-c2c1cc1nc(-c3ccccc3)sc1c2N(c1ccccc1)c1cccc(N(c2ccccc2)c2ccccc2)c1 ; CC1(C)c2cc3ccc4ccccc4c3cc2-c2c1cc1nc(-c3ccccc3)sc1c2N(c1cccc(N(c2ccccc2)c2ccccc2)c1)c1ccc2oc3ccccc3c2c1]

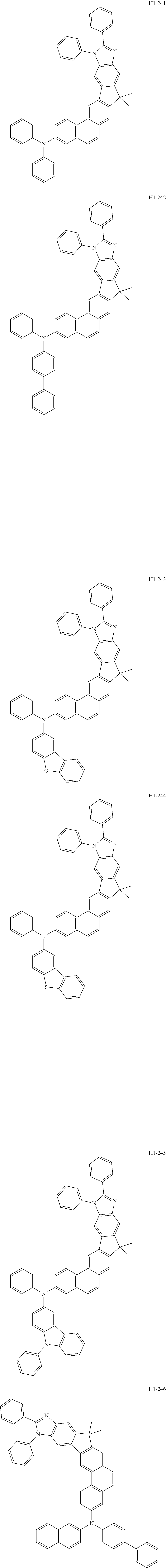

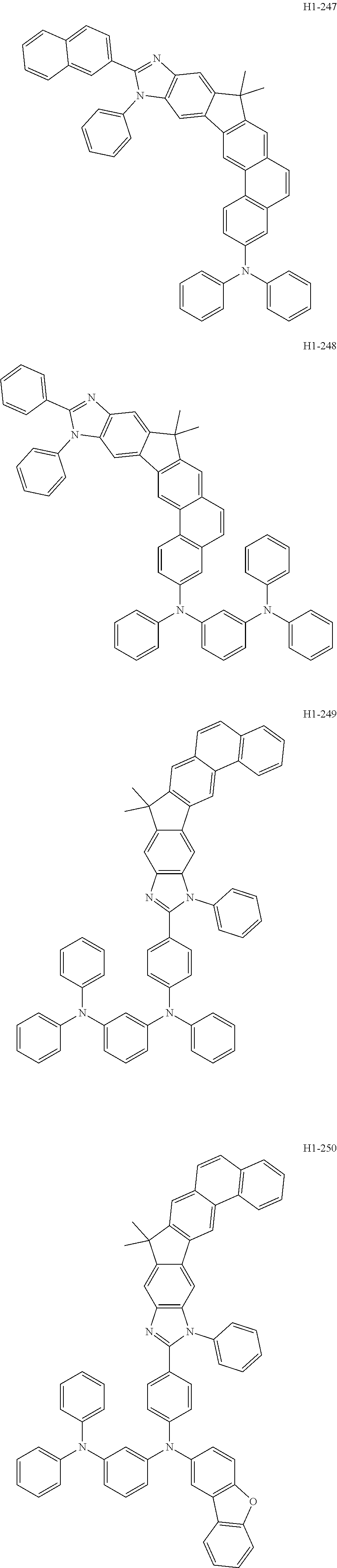 [SMILES: CC1(C)c2cc3ccc4cc(N(c5ccccc5)c5ccccc5)ccc4c3cc2-c2cc3c(cc21)nc(-c1ccc2ccccc2c1)n3-c1ccccc1 ; CC1(C)c2cc3ccc4cc(N(c5ccccc5)c5cccc(N(c6ccccc6)c6ccccc6)c5)ccc4c3cc2-c2cc3c(cc21)nc(-c1ccccc1)n3-c1ccccc1 ; CC1(C)c2cc3ccc4ccccc4c3cc2-c2cc3c(cc21)nc(-c1ccc(N(c2ccccc2)c2cccc(N(c4ccccc4)c4ccccc4)c2)cc1)n3-c1ccccc1 ; CC1(C)c2cc3ccc4ccccc4c3cc2-c2cc3c(cc21)nc(-c1ccc(N(c2cccc(N(c4ccccc4)c4ccccc4)c2)c2ccc4oc5ccccc5c4c2)cc1)n3-c1ccccc1]

[chemical structure]

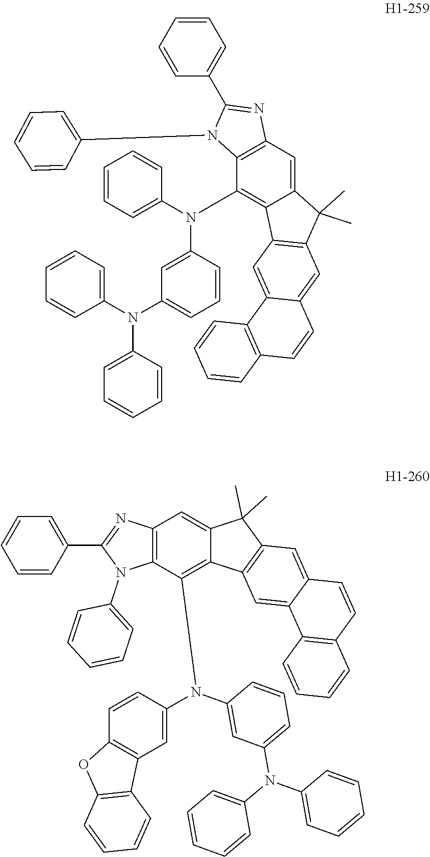 [SMILES: CC1(C)c2cc3ccc4ccccc4c3cc2-c2c1cc1nc(-c3ccccc3)n(-c3ccccc3)c1c2N(c1ccccc1)c1cccc(N(c2ccccc2)c2ccccc2)c1 ; CC1(C)c2cc3ccc4ccccc4c3cc2-c2c1cc1nc(-c3ccccc3)n(-c3ccccc3)c1c2N(c1cccc(N(c2ccccc2)c2ccccc2)c1)c1ccc2oc3ccccc3c2c1]

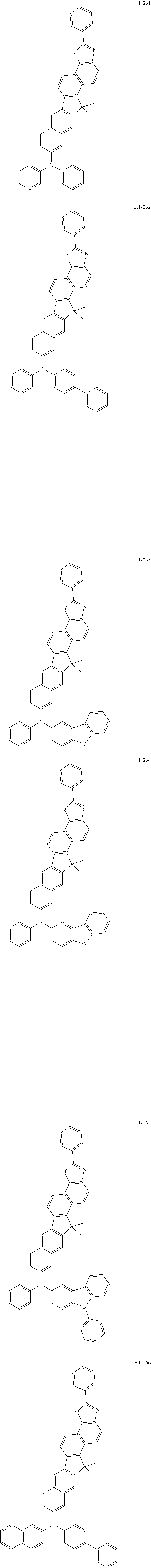

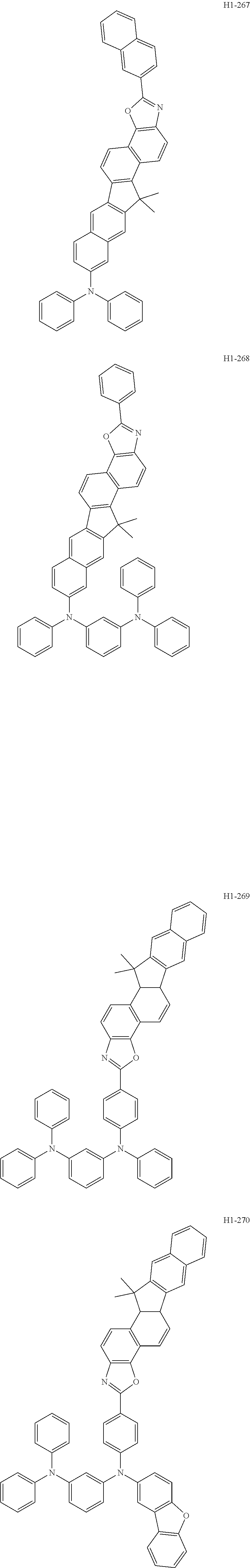 [SMILES: CC1(C)c2cc3cc(N(c4ccccc4)c4ccccc4)ccc3cc2-c2ccc3c(ccc4nc(-c5ccc6ccccc6c5)oc43)c21 ; CC1(C)c2cc3cc(N(c4ccccc4)c4cccc(N(c5ccccc5)c5ccccc5)c4)ccc3cc2-c2ccc3c(ccc4nc(-c5ccccc5)oc43)c21 ; CC1(C)c2cc3ccccc3cc2C2C=Cc3c(ccc4nc(-c5ccc(N(c6ccccc6)c6cccc(N(c7ccccc7)c7ccccc7)c6)cc5)oc34)C21 ; CC1(C)c2cc3ccccc3cc2C2C=Cc3c(ccc4nc(-c5ccc(N(c6cccc(N(c7ccccc7)c7ccccc7)c6)c6ccc7oc8ccccc8c7c6)cc5)oc34)C21]

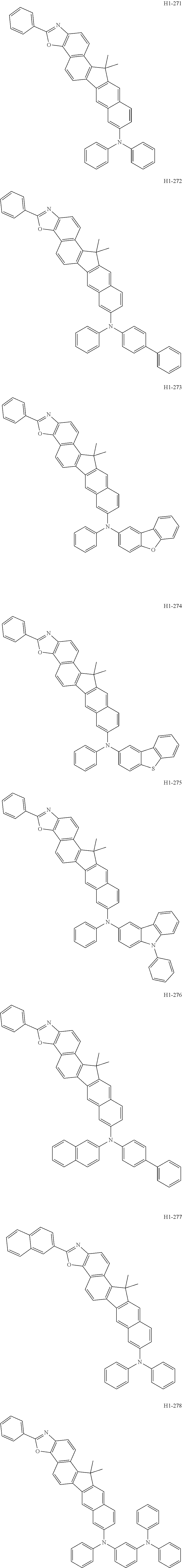

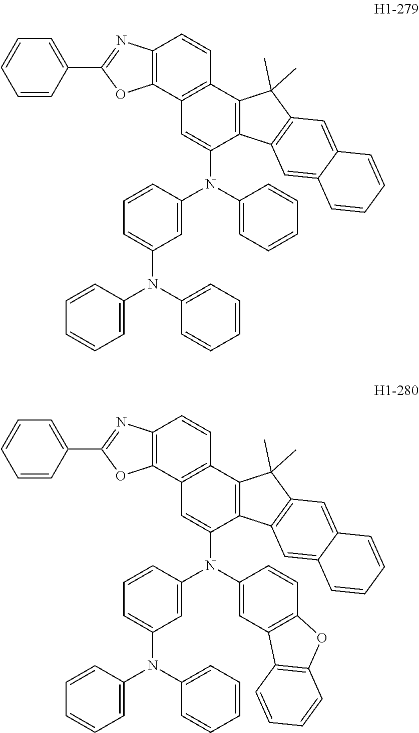 [SMILES: CC1(C)c2cc3ccccc3cc2-c2c(N(c3ccccc3)c3cccc(N(c4ccccc4)c4ccccc4)c3)cc3c(ccc4nc(-c5ccccc5)oc43)c21 ; CC1(C)c2cc3ccccc3cc2-c2c(N(c3cccc(N(c4ccccc4)c4ccccc4)c3)c3ccc4oc5ccccc5c4c3)cc3c(ccc4nc(-c5ccccc5)oc43)c21]

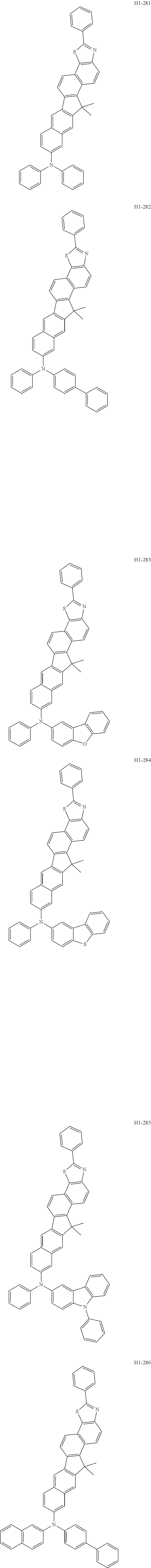

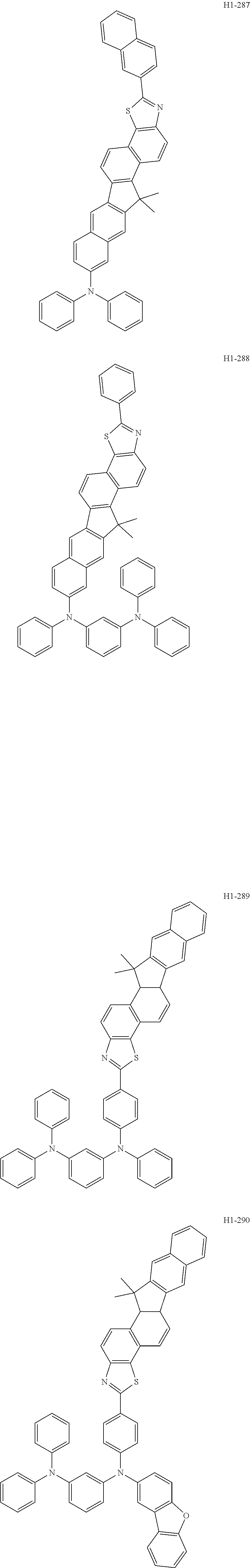 [SMILES: CC1(C)c2cc3cc(N(c4ccccc4)c4ccccc4)ccc3cc2-c2ccc3c(ccc4nc(-c5ccc6ccccc6c5)sc43)c21 ; CC1(C)c2cc3cc(N(c4ccccc4)c4cccc(N(c5ccccc5)c5ccccc5)c4)ccc3cc2-c2ccc3c(ccc4nc(-c5ccccc5)sc43)c21 ; CC1(C)c2cc3ccccc3cc2C2C=Cc3c(ccc4nc(-c5ccc(N(c6ccccc6)c6cccc(N(c7ccccc7)c7ccccc7)c6)cc5)sc34)C21 ; CC1(C)c2cc3ccccc3cc2C2C=Cc3c(ccc4nc(-c5ccc(N(c6cccc(N(c7ccccc7)c7ccccc7)c6)c6ccc7oc8ccccc8c7c6)cc5)sc34)C21]

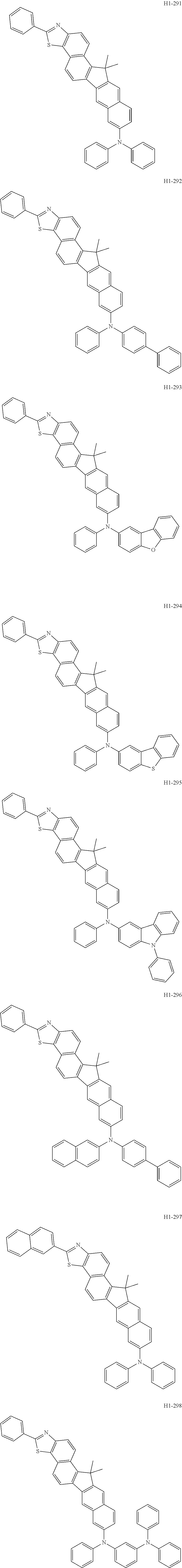

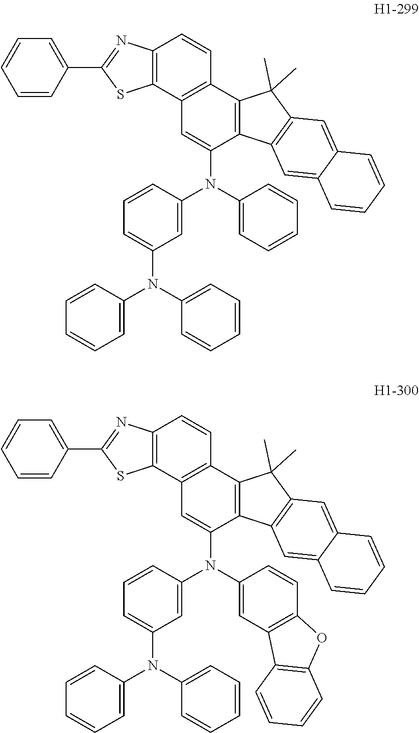 [SMILES: CC1(C)c2cc3ccccc3cc2-c2c(N(c3ccccc3)c3cccc(N(c4ccccc4)c4ccccc4)c3)cc3c(ccc4nc(-c5ccccc5)sc43)c21 ; CC1(C)c2cc3ccccc3cc2-c2c(N(c3cccc(N(c4ccccc4)c4ccccc4)c3)c3ccc4oc5ccccc5c4c3)cc3c(ccc4nc(-c5ccccc5)sc43)c21]

According to one embodiment of the present disclosure, the compound represented by formula 2 may be at least one selected from the following compounds.

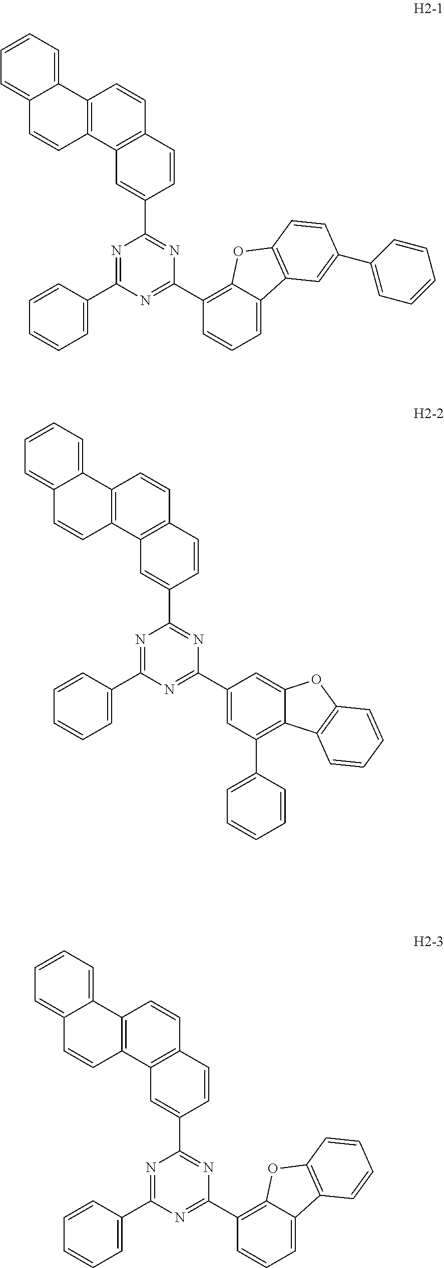 [SMILES: c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3cccc4c3oc3ccccc34)n2)cc1]

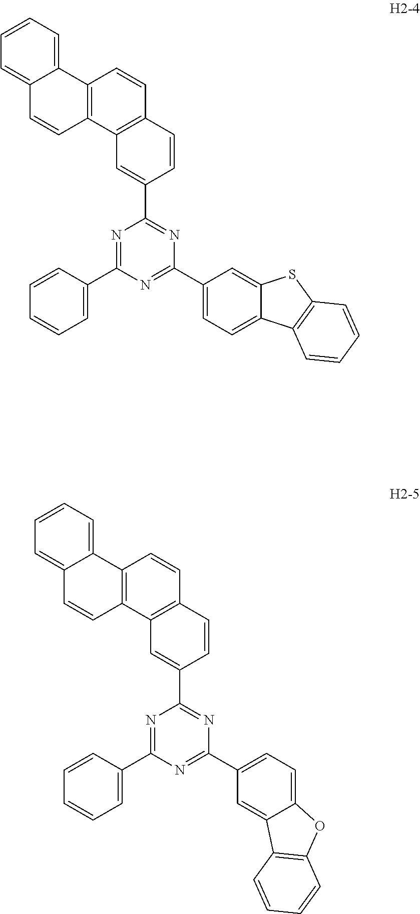 [SMILES: c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

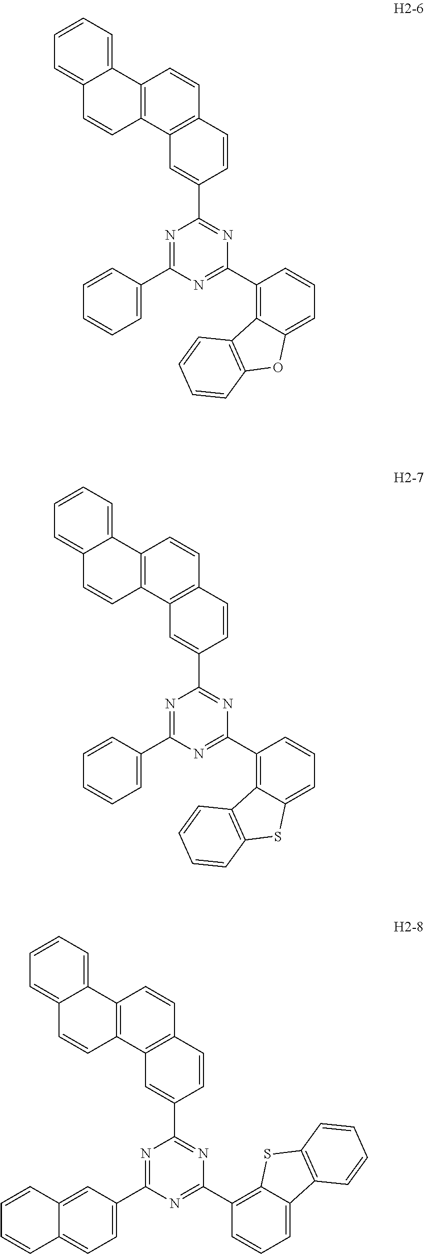 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5c4sc4ccccc45)n3)ccc2c1]

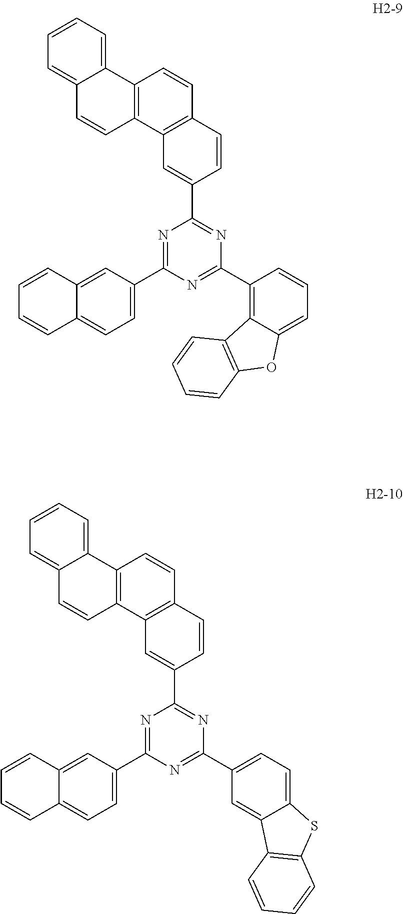 [SMILES: c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1]

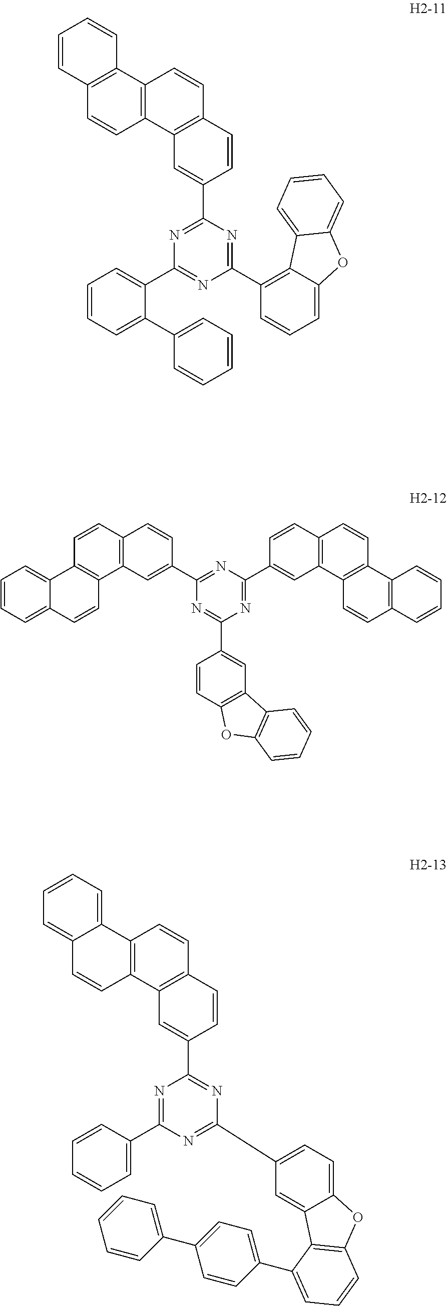 [SMILES: c1ccc(-c2ccccc2-c2nc(-c3ccc4ccc5c6ccccc6ccc5c4c3)nc(-c3cccc4oc5ccccc5c34)n2)cc1 ; c1ccc2c(c1)ccc1c3cc(-c4nc(-c5ccc6ccc7c8ccccc8ccc7c6c5)nc(-c5ccc6oc7ccccc7c6c5)n4)ccc3ccc21 ; c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1]

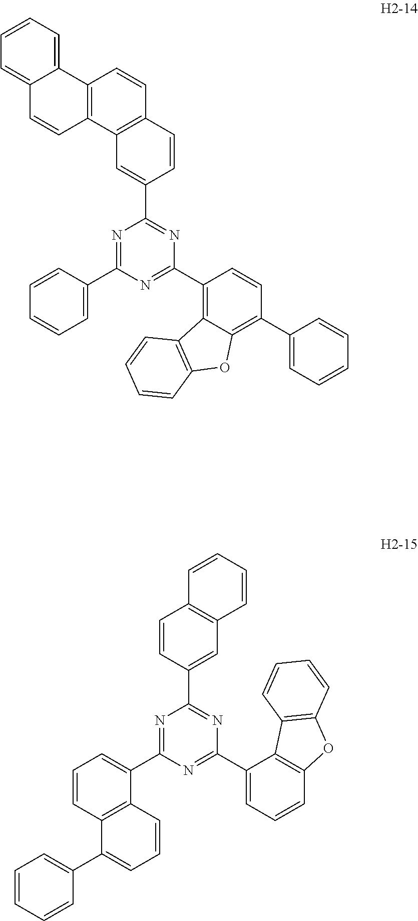 [SMILES: c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cccc3c(-c4nc(-c5ccc6ccccc6c5)nc(-c5cccc6oc7ccccc7c56)n4)cccc23)cc1]

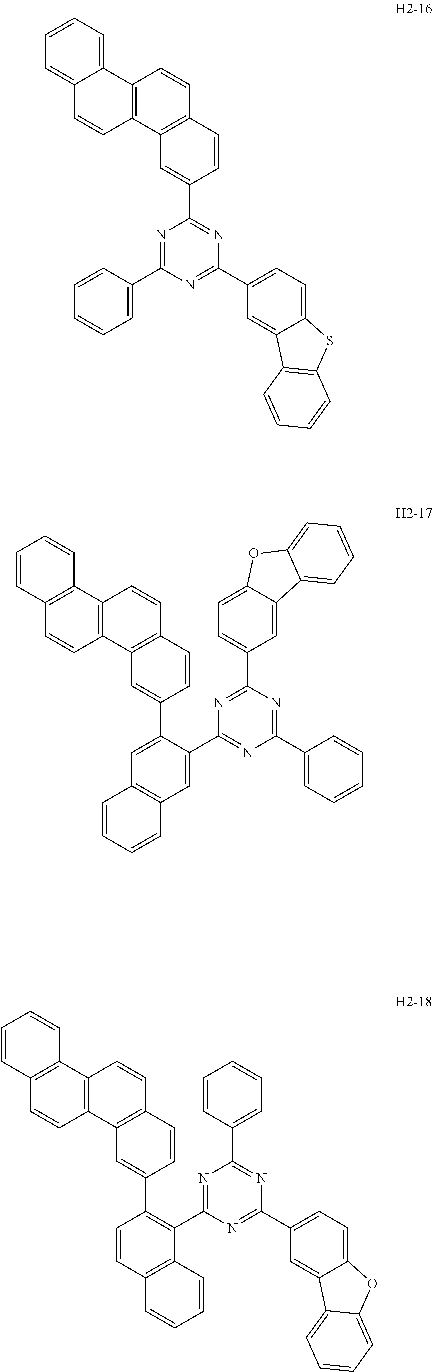 [SMILES: c1ccc(-c2nc(-c3ccc4ccc5c6ccccc6ccc5c4c3)nc(-c3ccc4sc5ccccc5c4c3)n2)cc1 ; c1ccc(-c2nc(-c3ccc4oc5ccccc5c4c3)nc(-c3cc4ccccc4cc3-c3ccc4ccc5c6ccccc6ccc5c4c3)n2)cc1 ; c1ccc(-c2nc(-c3ccc4oc5ccccc5c4c3)nc(-c3c(-c4ccc5ccc6c7ccccc7ccc6c5c4)ccc4ccccc34)n2)cc1]

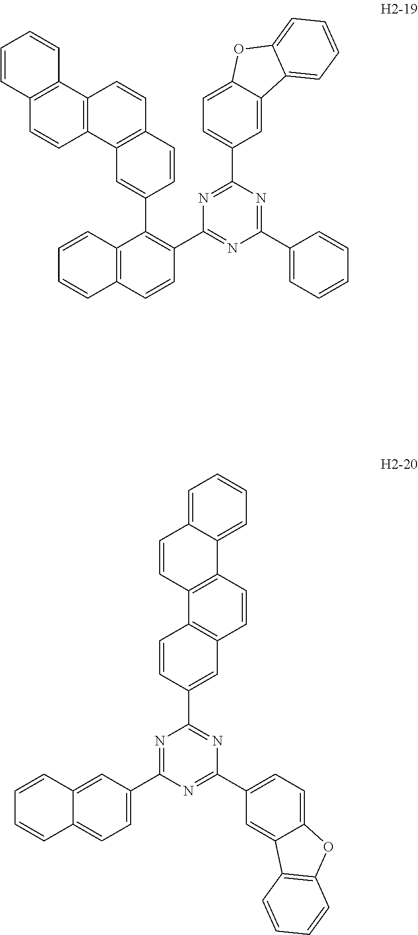 [SMILES: c1ccc(-c2nc(-c3ccc4oc5ccccc5c4c3)nc(-c3ccc4ccccc4c3-c3ccc4ccc5c6ccccc6ccc5c4c3)n2)cc1 ; c1ccc2cc(-c3nc(-c4ccc5c(ccc6c7ccccc7ccc56)c4)nc(-c4ccc5oc6ccccc6c5c4)n3)ccc2c1]

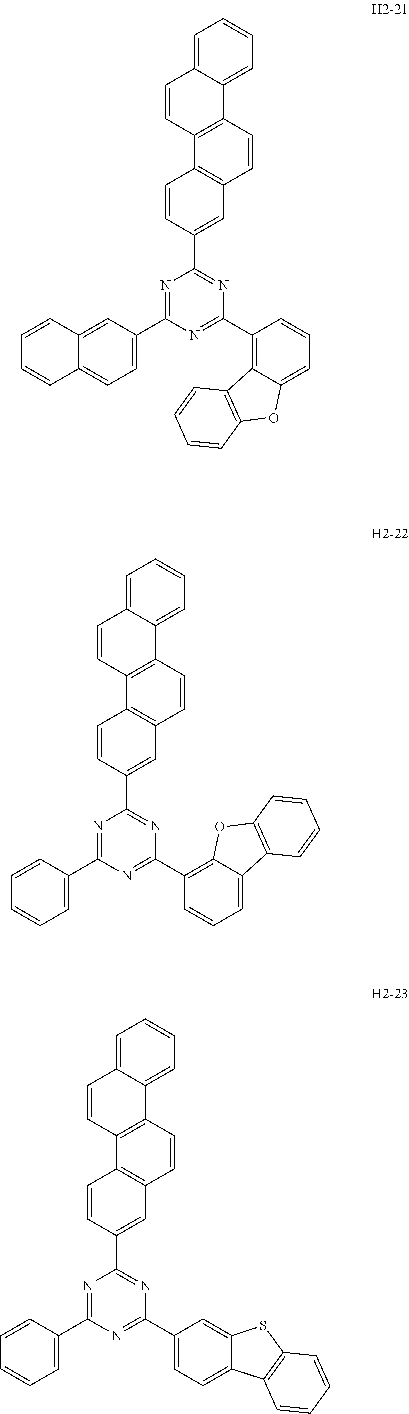 [SMILES: c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1]

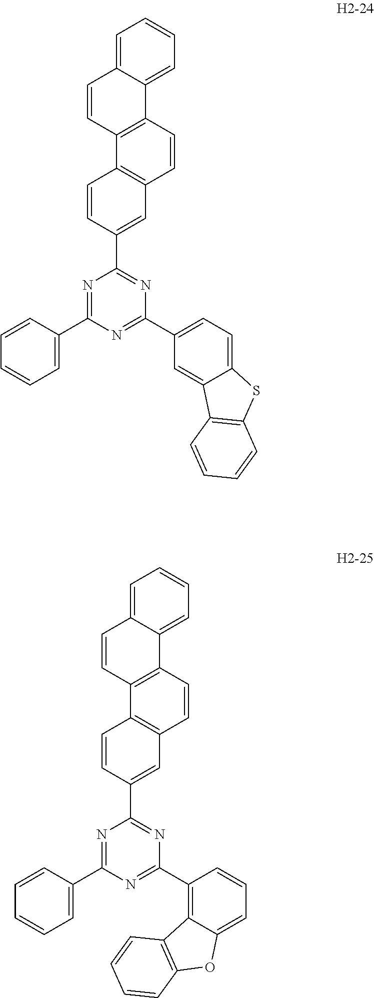 [SMILES: c1ccc(-c2nc(-c3ccc4c(ccc5c6ccccc6ccc45)c3)nc(-c3ccc4sc5ccccc5c4c3)n2)cc1 ; c1ccc(-c2nc(-c3ccc4c(ccc5c6ccccc6ccc45)c3)nc(-c3cccc4oc5ccccc5c34)n2)cc1]

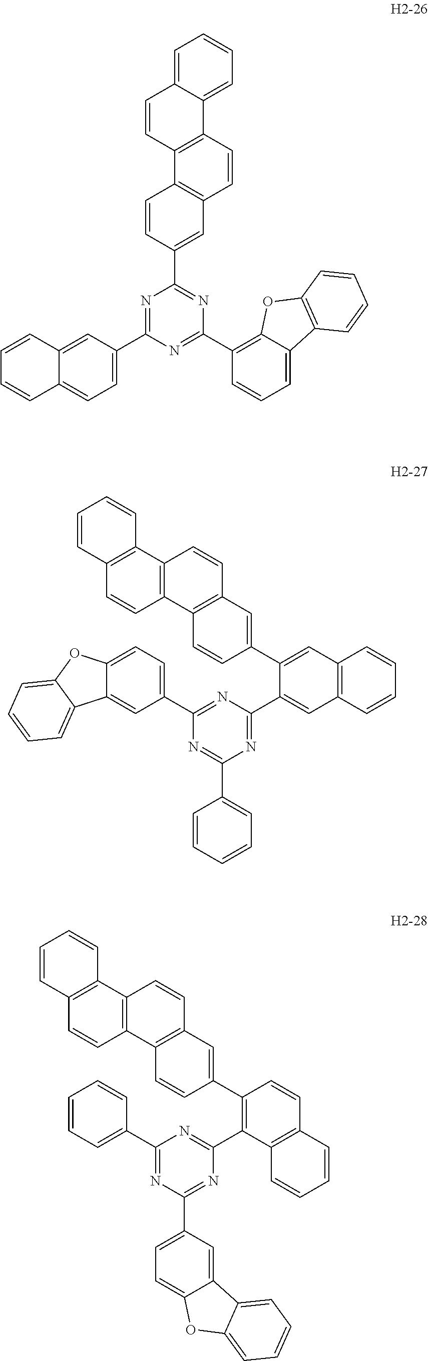 [SMILES: c1ccc2cc(-c3nc(-c4ccc5c(ccc6c7ccccc7ccc56)c4)nc(-c4cccc5c4oc4ccccc45)n3)ccc2c1 ; c1ccc(-c2nc(-c3ccc4oc5ccccc5c4c3)nc(-c3cc4ccccc4cc3-c3ccc4c(ccc5c6ccccc6ccc45)c3)n2)cc1 ; c1ccc(-c2nc(-c3ccc4oc5ccccc5c4c3)nc(-c3c(-c4ccc5c(ccc6c7ccccc7ccc56)c4)ccc4ccccc34)n2)cc1]

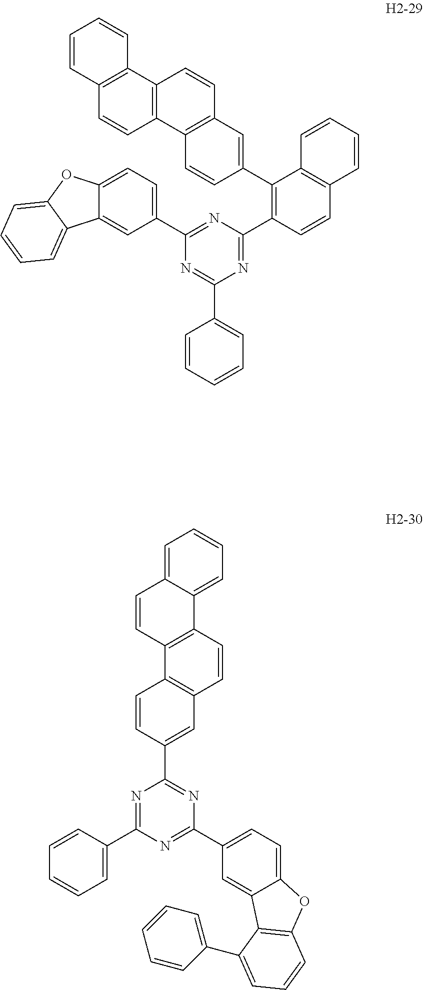 [SMILES: c1ccc(-c2nc(-c3ccc4oc5ccccc5c4c3)nc(-c3ccc4ccccc4c3-c3ccc4c(ccc5c6ccccc6ccc45)c3)n2)cc1 ; c1ccc(-c2nc(-c3ccc4c(ccc5c6ccccc6ccc45)c3)nc(-c3ccc4oc5cccc(-c6ccccc6)c5c4c3)n2)cc1]

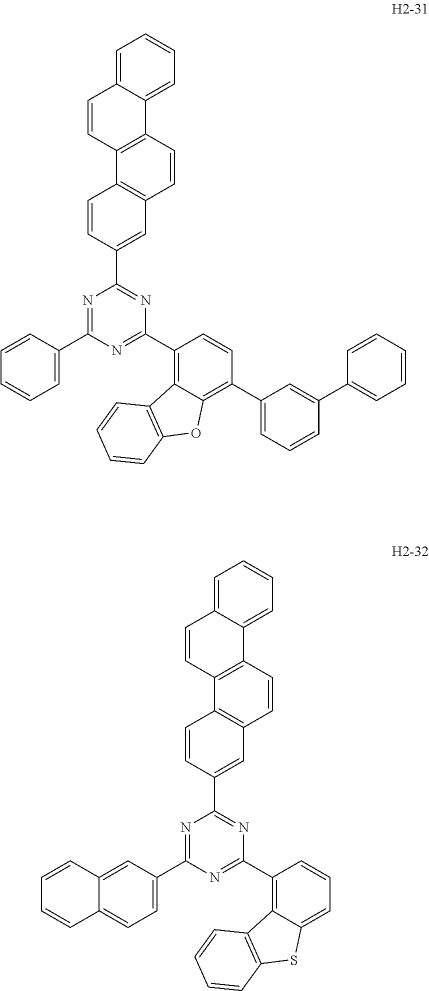 [SMILES: c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4cccc5sc6ccccc6c45)n3)ccc2c1]

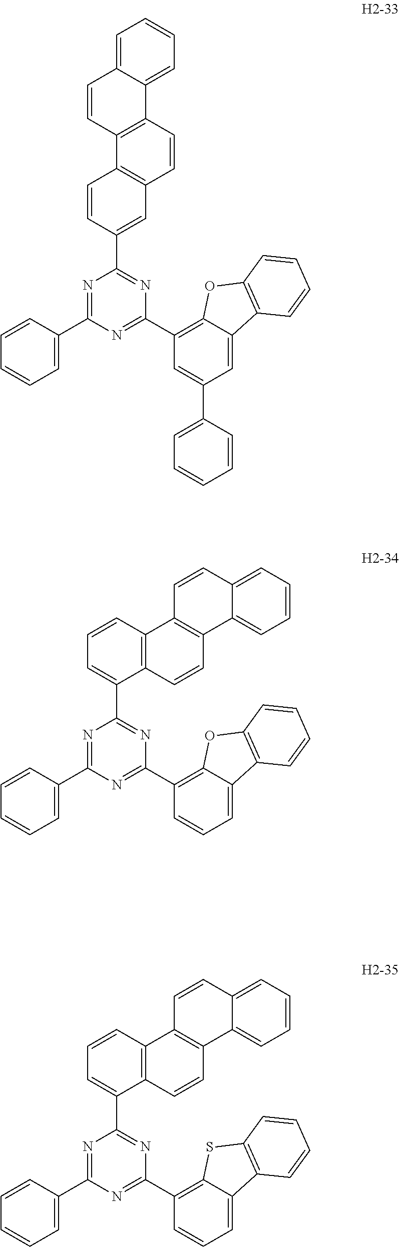 [SMILES: c1ccc(-c2cc(-c3nc(-c4ccccc4)nc(-c4ccc5c(ccc6c7ccccc7ccc56)c4)n3)c3oc4ccccc4c3c2)cc1 ; c1ccc(-c2nc(-c3cccc4c3ccc3c5ccccc5ccc43)nc(-c3cccc4c3oc3ccccc34)n2)cc1 ; c1ccc(-c2nc(-c3cccc4c3ccc3c5ccccc5ccc43)nc(-c3cccc4c3sc3ccccc34)n2)cc1]

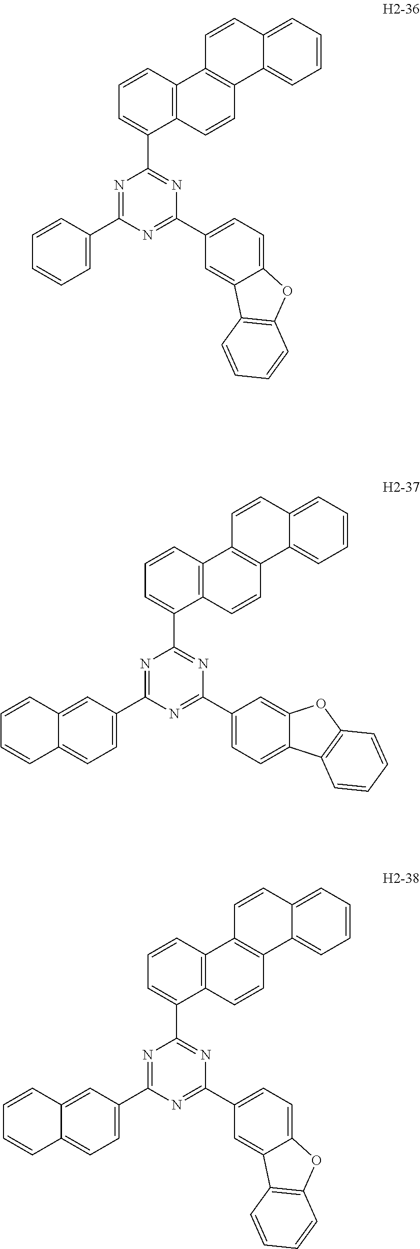 [SMILES: c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1 ; c1ccc2cc(-c3nc(-c4ccc5oc6ccccc6c5c4)nc(-c4cccc5c4ccc4c6ccccc6ccc54)n3)ccc2c1]

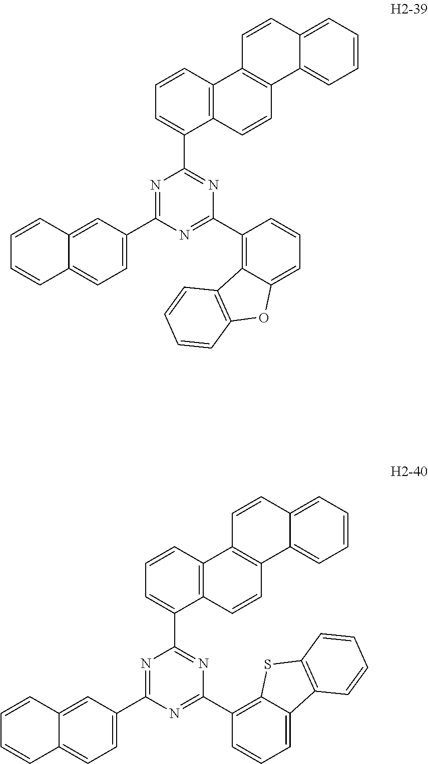 [SMILES: c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1]

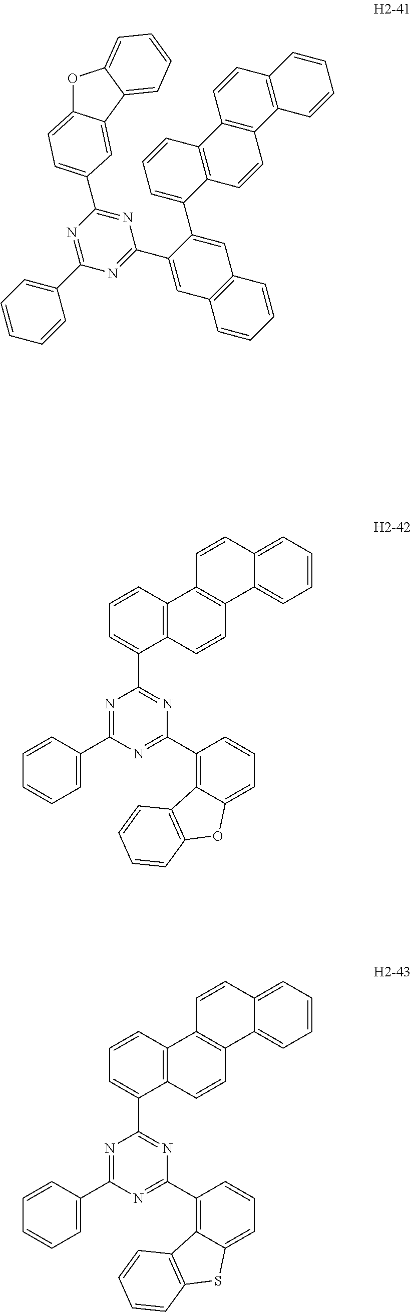 [SMILES: c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1 ; c1ccc(-c2nc(-c3cccc4c3ccc3c5ccccc5ccc43)nc(-c3cccc4sc5ccccc5c34)n2)cc1]

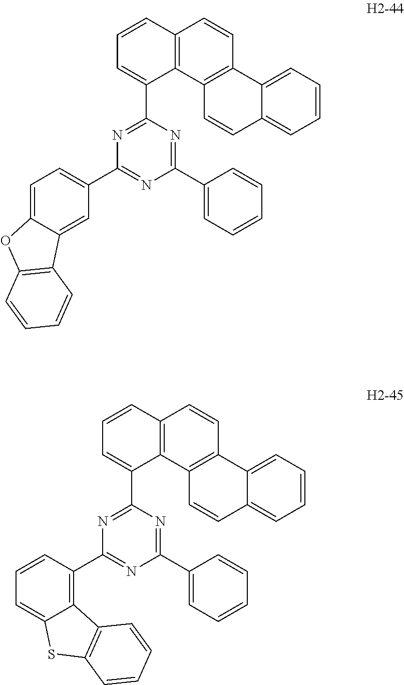 [SMILES: c1ccc(-c2nc(-c3ccc4oc5ccccc5c4c3)nc(-c3cccc4ccc5c6ccccc6ccc5c34)n2)cc1 ; c1ccc(-c2nc(-c3cccc4ccc5c6ccccc6ccc5c34)nc(-c3cccc4sc5ccccc5c34)n2)cc1]

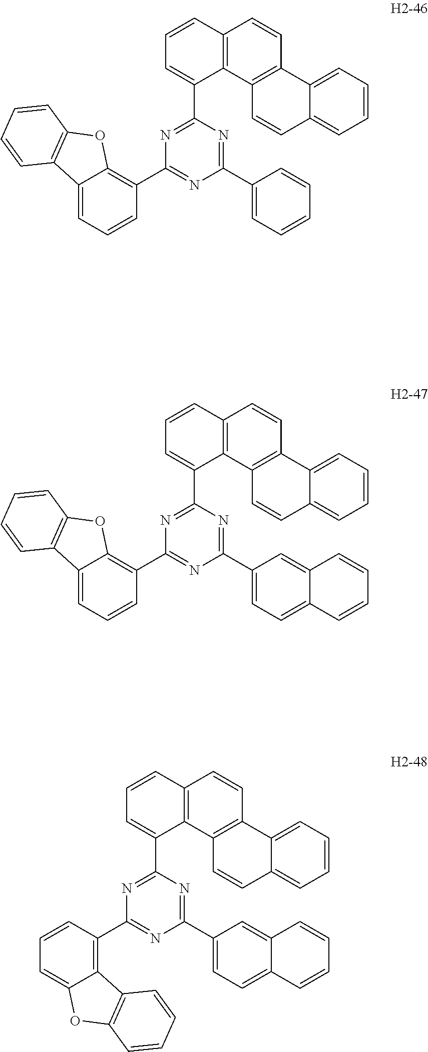 [SMILES: c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1 ; c1ccc2cc(-c3nc(-c4cccc5ccc6c7ccccc7ccc6c45)nc(-c4cccc5oc6ccccc6c45)n3)ccc2c1]

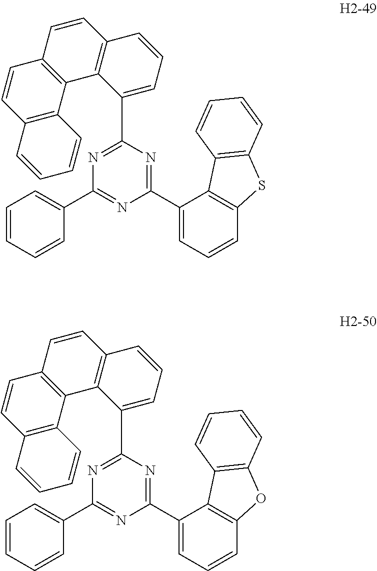 [SMILES: c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1]

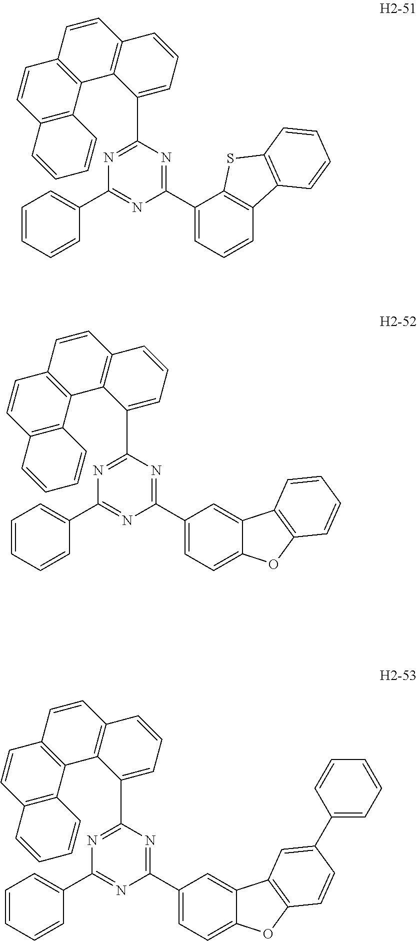 [SMILES: c1ccc(-c2nc(-c3cccc4c3sc3ccccc34)nc(-c3cccc4ccc5ccc6ccccc6c5c34)n2)cc1 ; c1ccc(-c2nc(-c3ccc4oc5ccccc5c4c3)nc(-c3cccc4ccc5ccc6ccccc6c5c34)n2)cc1 ; c1ccc(-c2ccc3oc4ccc(-c5nc(-c6ccccc6)nc(-c6cccc7ccc8ccc9ccccc9c8c67)n5)cc4c3c2)cc1]

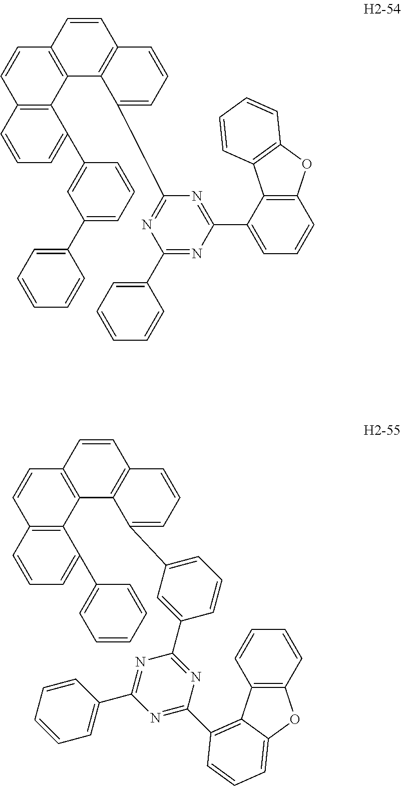 [SMILES: c1ccc(-c2cccc(-c3cccc4ccc5ccc6cccc(-c7nc(-c8ccccc8)nc(-c8cccc9oc%10ccccc%10c89)n7)c6c5c34)c2)cc1 ; c1ccc(-c2nc(-c3cccc(-c4cccc5ccc6ccc7cccc(-c8ccccc8)c7c6c45)c3)nc(-c3cccc4oc5ccccc5c34)n2)cc1]

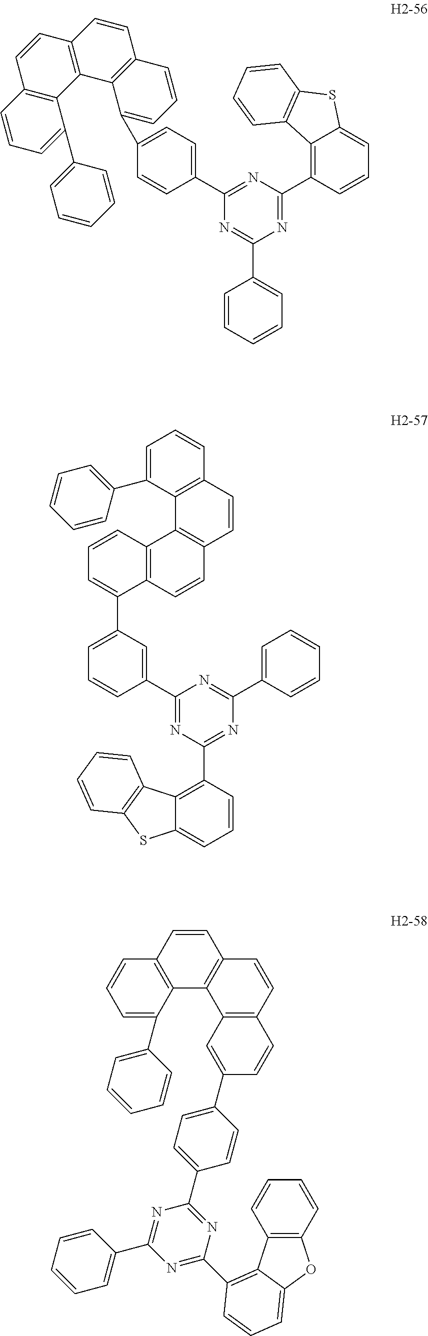 [SMILES: c1ccc(-c2nc(-c3ccc(-c4cccc5ccc6ccc7cccc(-c8ccccc8)c7c6c45)cc3)nc(-c3cccc4sc5ccccc5c34)n2)cc1 ; c1ccc(-c2nc(-c3cccc(-c4cccc5c4ccc4ccc6cccc(-c7ccccc7)c6c45)c3)nc(-c3cccc4sc5ccccc5c34)n2)cc1 ; c1ccc(-c2nc(-c3ccc(-c4ccc5ccc6ccc7cccc(-c8ccccc8)c7c6c5c4)cc3)nc(-c3cccc4oc5ccccc5c34)n2)cc1]

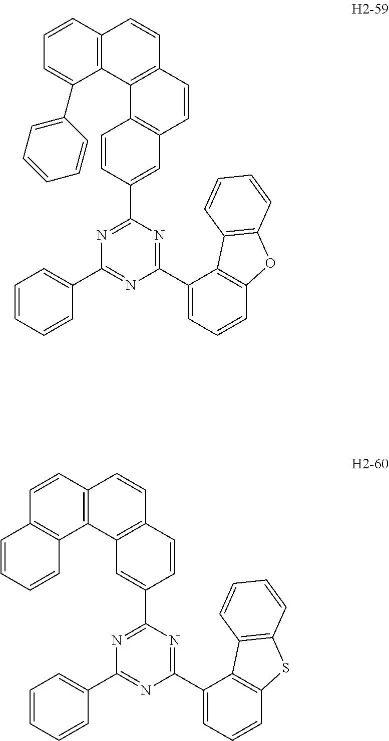 [SMILES: c1ccc(-c2nc(-c3ccc4c(ccc5ccc6cccc(-c7ccccc7)c6c54)c3)nc(-c3cccc4oc5ccccc5c34)n2)cc1 ; c1ccc(-c2nc(-c3ccc4ccc5ccc6ccccc6c5c4c3)nc(-c3cccc4sc5ccccc5c34)n2)cc1]

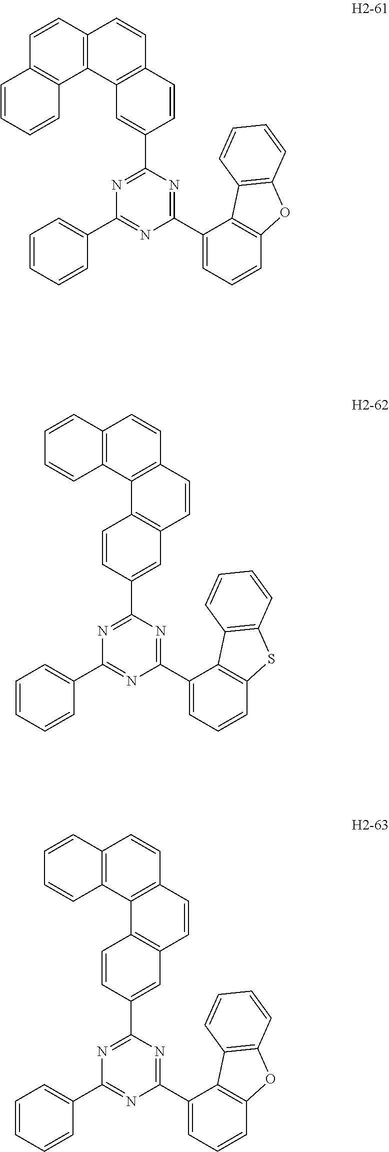 [SMILES: c1ccc(-c2nc(-c3ccc4ccc5ccc6ccccc6c5c4c3)nc(-c3cccc4oc5ccccc5c34)n2)cc1 ; c1ccc(-c2nc(-c3ccc4c(ccc5ccc6ccccc6c54)c3)nc(-c3cccc4sc5ccccc5c34)n2)cc1 ; c1ccc(-c2nc(-c3ccc4c(ccc5ccc6ccccc6c54)c3)nc(-c3cccc4oc5ccccc5c34)n2)cc1]

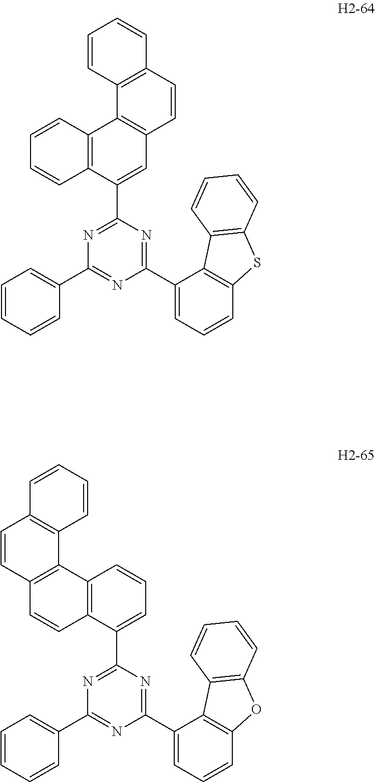 [SMILES: c1ccc(-c2nc(-c3cc4ccc5ccccc5c4c4ccccc34)nc(-c3cccc4sc5ccccc5c34)n2)cc1 ; c1ccc(-c2nc(-c3cccc4c3ccc3ccc5ccccc5c34)nc(-c3cccc4oc5ccccc5c34)n2)cc1]

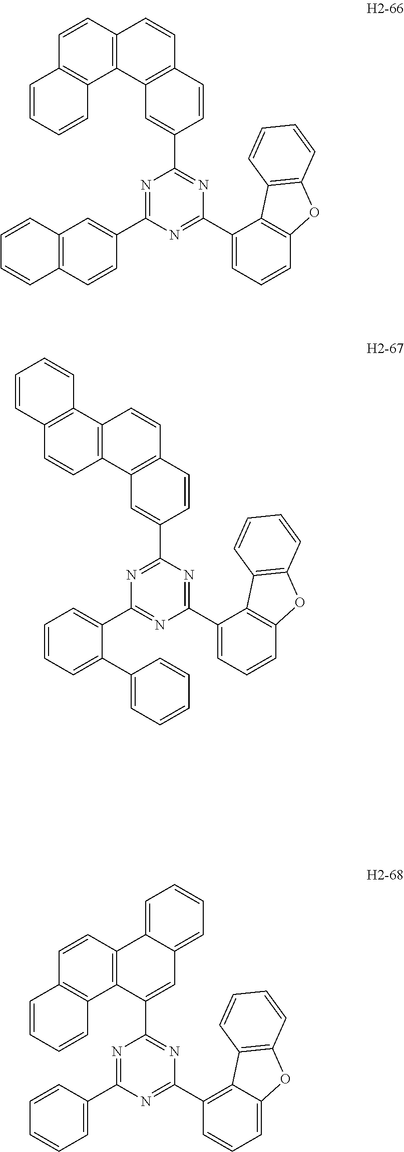 [SMILES: c1ccc2cc(-c3nc(-c4ccc5ccc6ccc7ccccc7c6c5c4)nc(-c4cccc5oc6ccccc6c45)n3)ccc2c1 ; c1ccc(-c2ccccc2-c2nc(-c3ccc4ccc5c6ccccc6ccc5c4c3)nc(-c3cccc4oc5ccccc5c34)n2)cc1 ; c1ccc(-c2nc(-c3cccc4oc5ccccc5c34)nc(-c3cc4ccccc4c4ccc5ccccc5c34)n2)cc1]

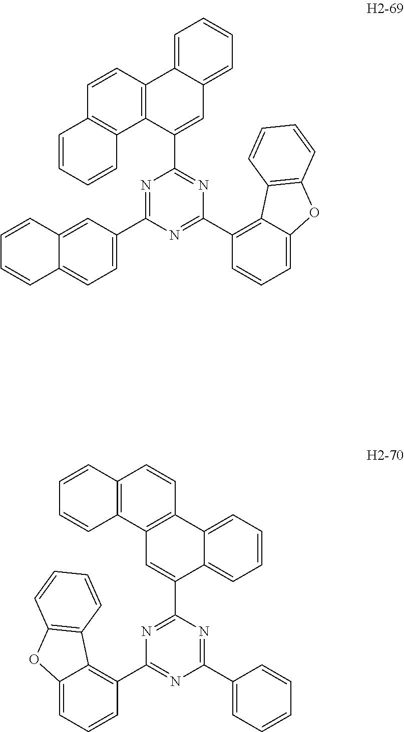 [SMILES: c1ccc2cc(-c3nc(-c4cccc5oc6ccccc6c45)nc(-c4cc5ccccc5c5ccc6ccccc6c45)n3)ccc2c1 ; c1ccc(-c2nc(-c3cc4c5ccccc5ccc4c4ccccc34)nc(-c3cccc4oc5ccccc5c34)n2)cc1]

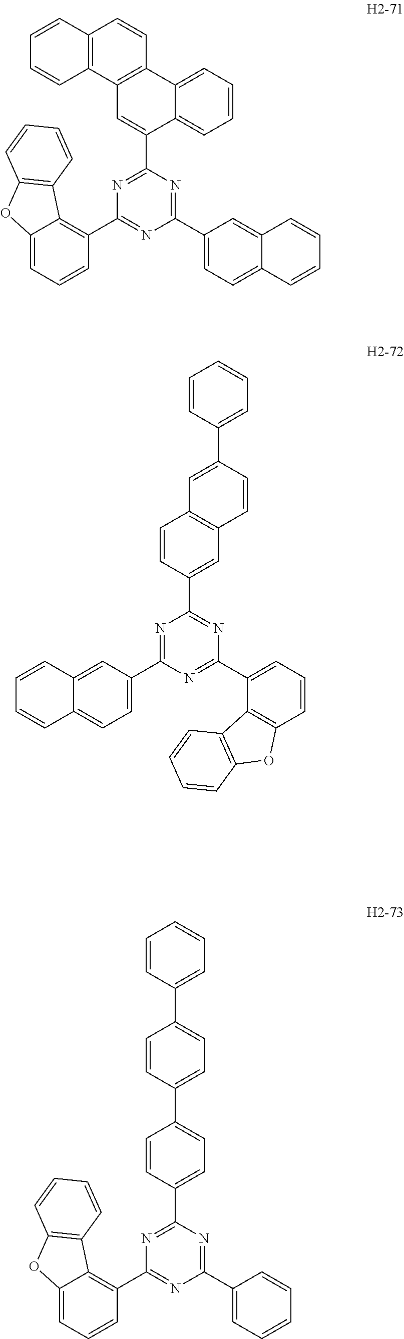 [SMILES: c1ccc2cc(-c3nc(-c4cc5c6ccccc6ccc5c5ccccc45)nc(-c4cccc5oc6ccccc6c45)n3)ccc2c1 ; c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1]

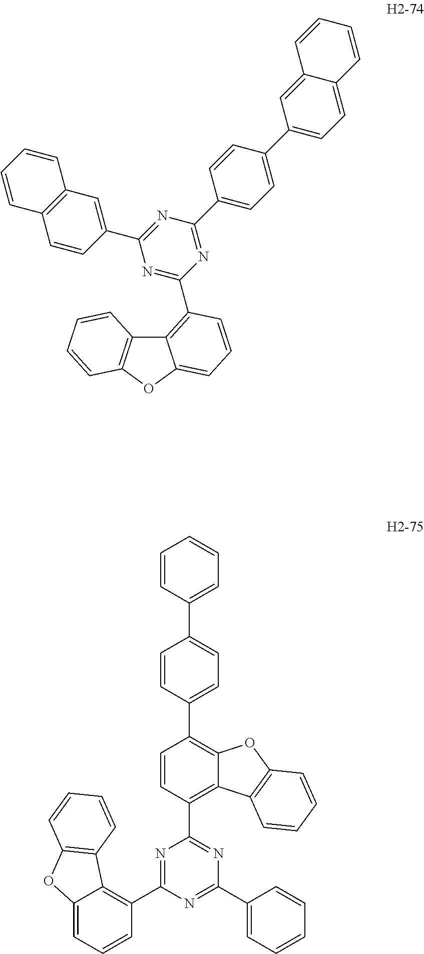 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3ccc(-c4nc(-c5ccccc5)nc(-c5cccc6oc7ccccc7c56)n4)c4c3oc3ccccc34)cc2)cc1]

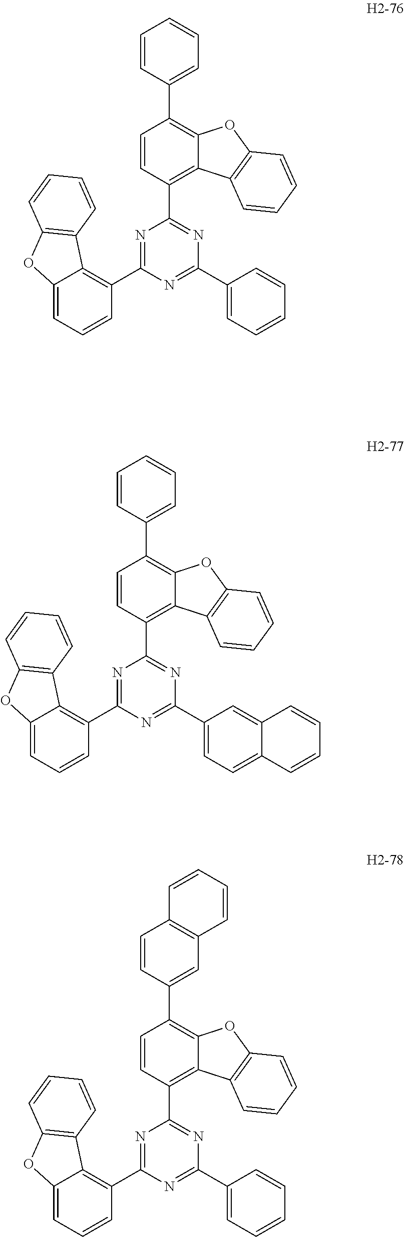 [SMILES: c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1 ; c1ccc(-c2nc(-c3cccc4oc5ccccc5c34)nc(-c3ccc(-c4ccc5ccccc5c4)c4oc5ccccc5c34)n2)cc1]

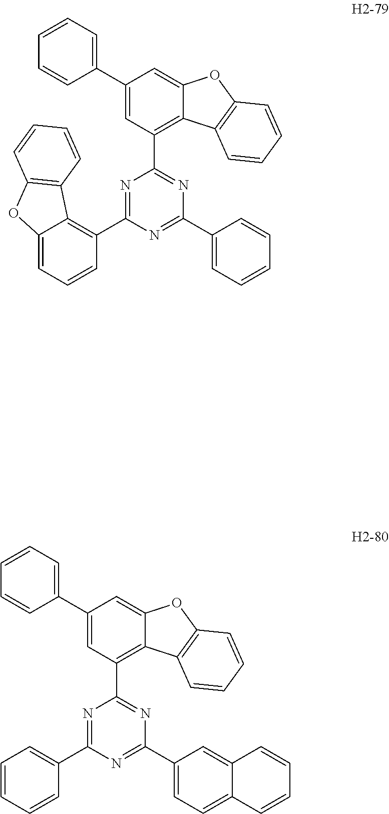 [SMILES: c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccccc4)nc(-c4ccc5ccccc5c4)n3)c3c(c2)oc2ccccc23)cc1]

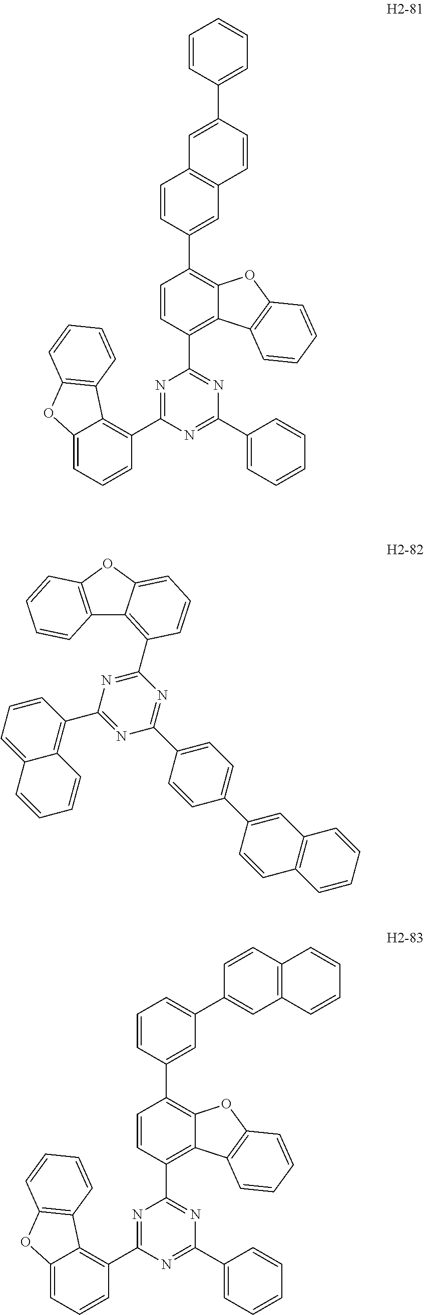 [SMILES: c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc2cc(-c3ccc(-c4nc(-c5cccc6ccccc56)nc(-c5cccc6oc7ccccc7c56)n4)cc3)ccc2c1 ; c1ccc(-c2nc(-c3cccc4oc5ccccc5c34)nc(-c3ccc(-c4cccc(-c5ccc6ccccc6c5)c4)c4oc5ccccc5c34)n2)cc1]

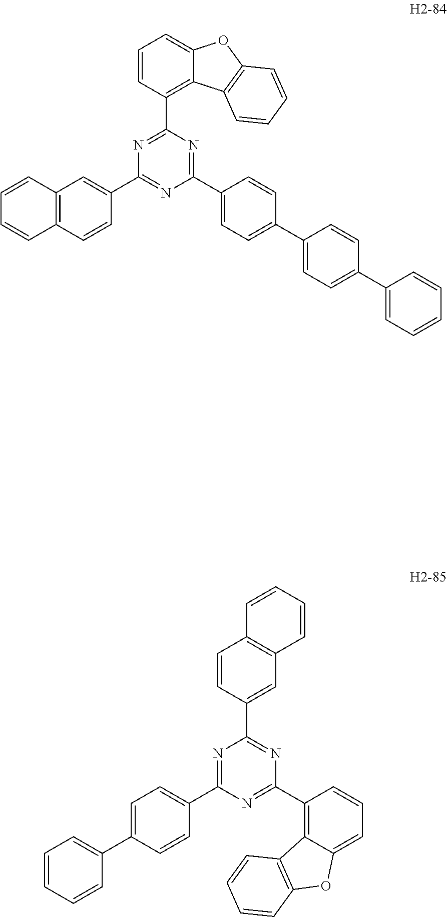 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3nc(-c4ccc5ccccc5c4)nc(-c4cccc5oc6ccccc6c45)n3)cc2)cc1]

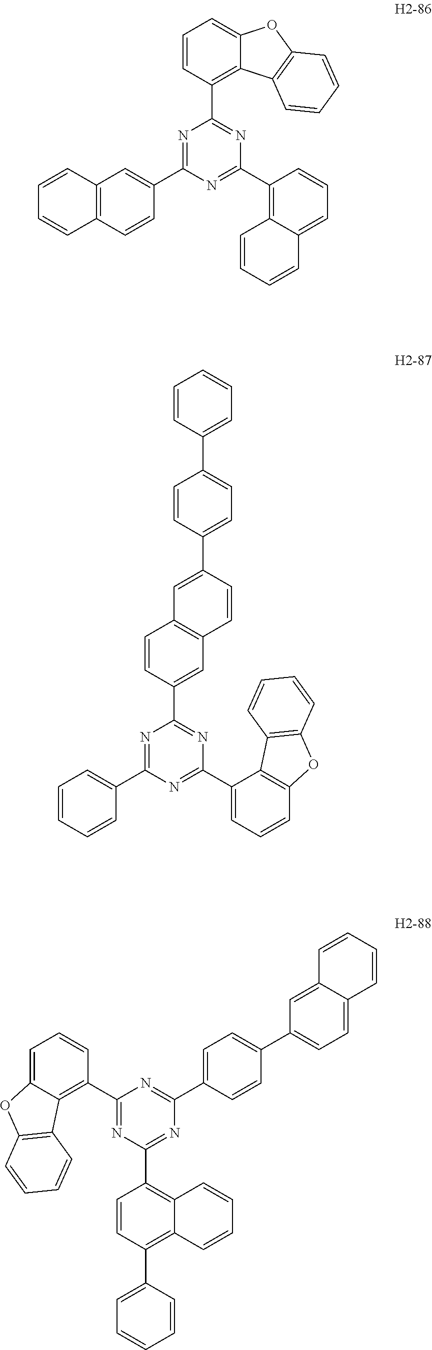 [SMILES: c1ccc2cc(-c3nc(-c4cccc5ccccc45)nc(-c4cccc5oc6ccccc6c45)n3)ccc2c1 ; c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)c3ccccc23)cc1]

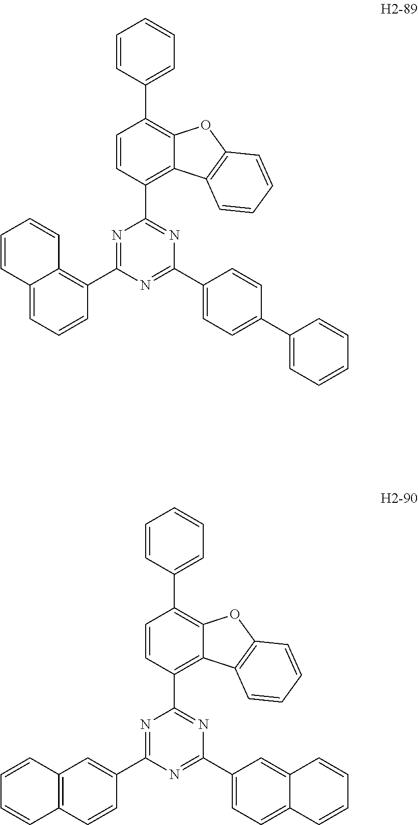 [SMILES: c1ccc(-c2ccc(-c3nc(-c4cccc5ccccc45)nc(-c4ccc(-c5ccccc5)c5oc6ccccc6c45)n3)cc2)cc1 ; c1ccc(-c2ccc(-c3nc(-c4ccc5ccccc5c4)nc(-c4ccc5ccccc5c4)n3)c3c2oc2ccccc23)cc1]

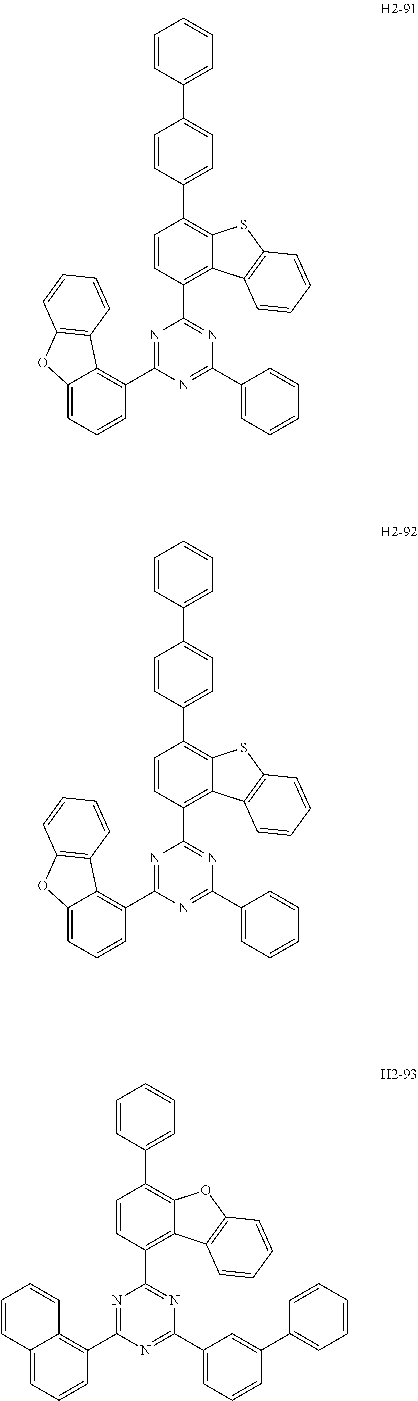 [SMILES: c1ccc(-c2ccc(-c3ccc(-c4nc(-c5ccccc5)nc(-c5cccc6oc7ccccc7c56)n4)c4c3sc3ccccc34)cc2)cc1 ; c1ccc(-c2ccc(-c3ccc(-c4nc(-c5ccccc5)nc(-c5cccc6oc7ccccc7c56)n4)c4c3sc3ccccc34)cc2)cc1 ; c1ccc(-c2cccc(-c3nc(-c4cccc5ccccc45)nc(-c4ccc(-c5ccccc5)c5oc6ccccc6c45)n3)c2)cc1]

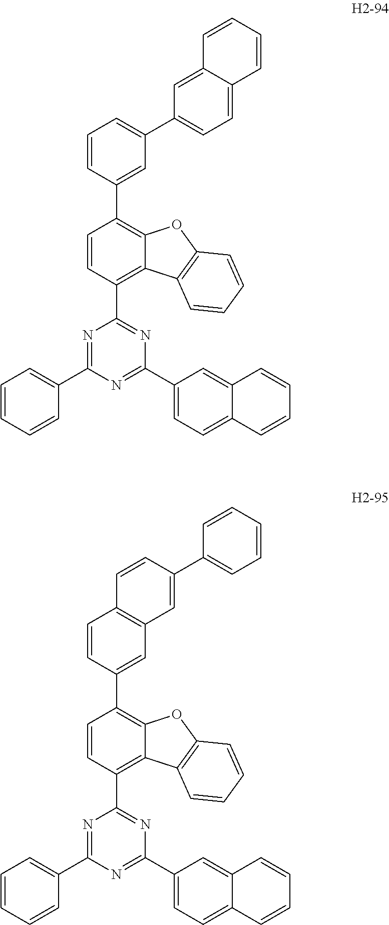 [SMILES: c1ccc(-c2nc(-c3ccc4ccccc4c3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccc7ccccc7c6)n5)c5c4oc4ccccc45)cc3c2)cc1]

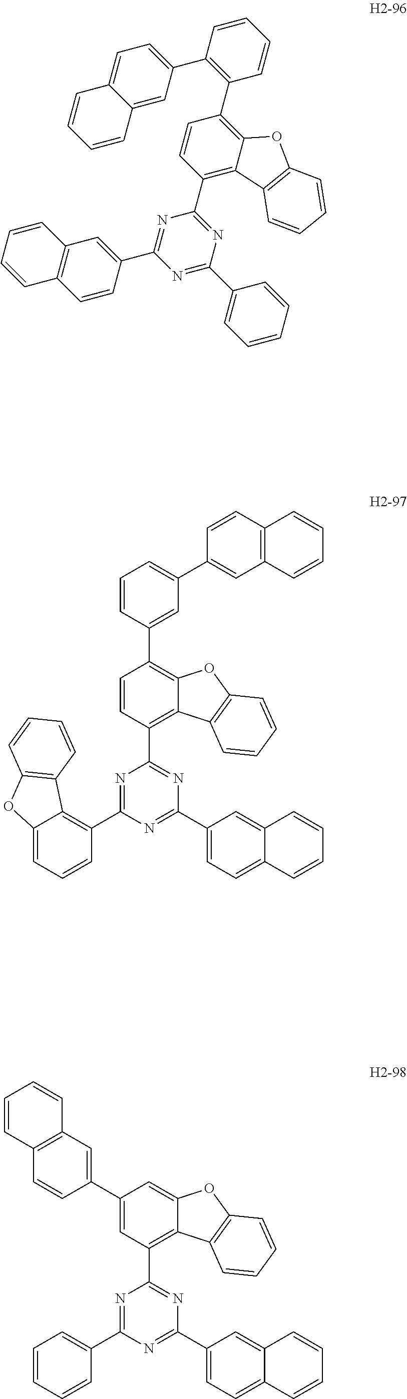 [SMILES: c1ccc(-c2nc(-c3ccc4ccccc4c3)nc(-c3ccc(-c4ccccc4-c4ccc5ccccc5c4)c4oc5ccccc5c34)n2)cc1 ; c1cc(-c2ccc3ccccc3c2)cc(-c2ccc(-c3nc(-c4ccc5ccccc5c4)nc(-c4cccc5oc6ccccc6c45)n3)c3c2oc2ccccc23)c1 ; c1ccc(-c2nc(-c3ccc4ccccc4c3)nc(-c3cc(-c4ccc5ccccc5c4)cc4oc5ccccc5c34)n2)cc1]

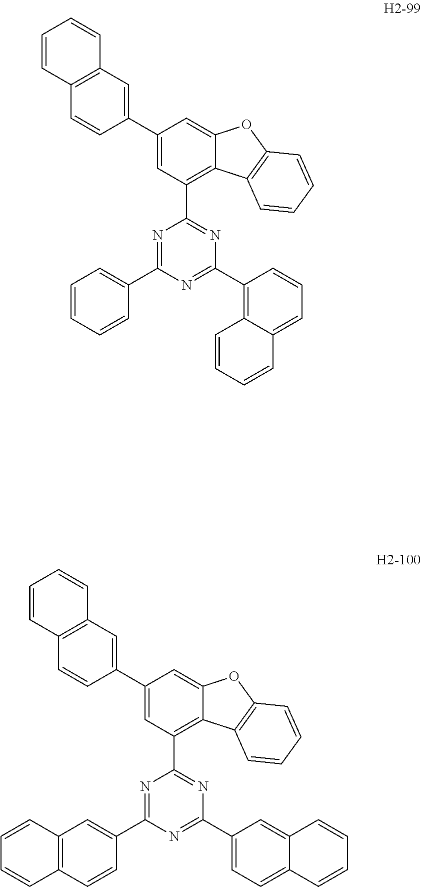 [SMILES: c1ccc(-c2nc(-c3cccc4ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5ccc6ccccc6c5)n4)c4c(c3)oc3ccccc34)ccc2c1]

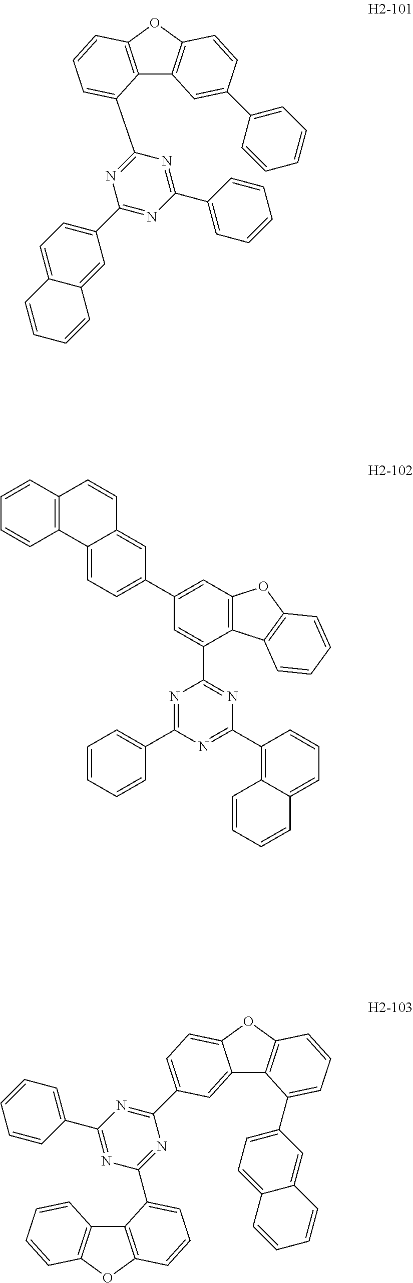 [SMILES: c1ccc(-c2ccc3oc4cccc(-c5nc(-c6ccccc6)nc(-c6ccc7ccccc7c6)n5)c4c3c2)cc1 ; c1ccc(-c2nc(-c3cccc4ccccc34)nc(-c3cc(-c4ccc5c(ccc6ccccc65)c4)cc4oc5ccccc5c34)n2)cc1 ; c1ccc(-c2nc(-c3ccc4oc5cccc(-c6ccc7ccccc7c6)c5c4c3)nc(-c3cccc4oc5ccccc5c34)n2)cc1]

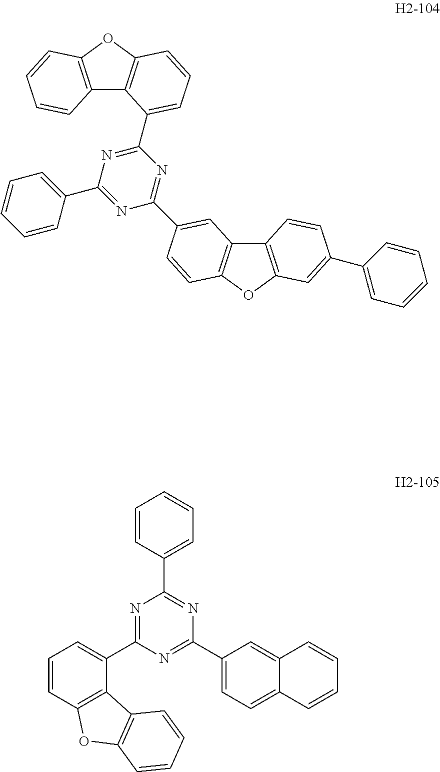 [SMILES: c1ccc(-c2ccc3c(c2)oc2ccc(-c4nc(-c5ccccc5)nc(-c5cccc6oc7ccccc7c56)n4)cc23)cc1 ; c1ccc(-c2nc(-c3ccc4ccccc4c3)nc(-c3cccc4oc5ccccc5c34)n2)cc1]

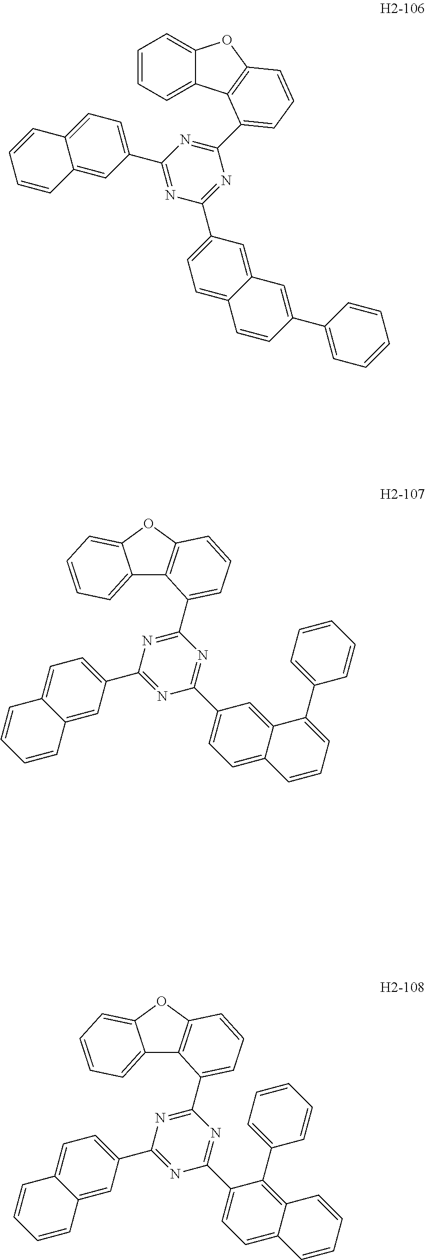 [SMILES: c1ccc(-c2ccc3ccc(-c4nc(-c5ccc6ccccc6c5)nc(-c5cccc6oc7ccccc7c56)n4)cc3c2)cc1 ; c1ccc(-c2cccc3ccc(-c4nc(-c5ccc6ccccc6c5)nc(-c5cccc6oc7ccccc7c56)n4)cc23)cc1 ; c1ccc(-c2c(-c3nc(-c4ccc5ccccc5c4)nc(-c4cccc5oc6ccccc6c45)n3)ccc3ccccc23)cc1]

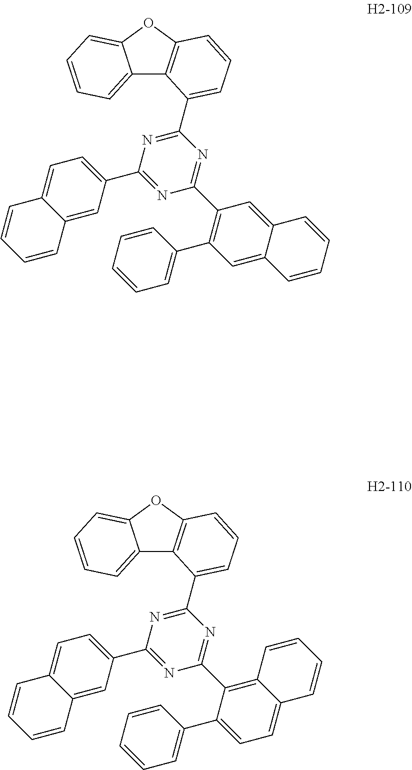 [SMILES: c1ccc(-c2cc3ccccc3cc2-c2nc(-c3ccc4ccccc4c3)nc(-c3cccc4oc5ccccc5c34)n2)cc1 ; c1ccc(-c2ccc3ccccc3c2-c2nc(-c3ccc4ccccc4c3)nc(-c3cccc4oc5ccccc5c34)n2)cc1]

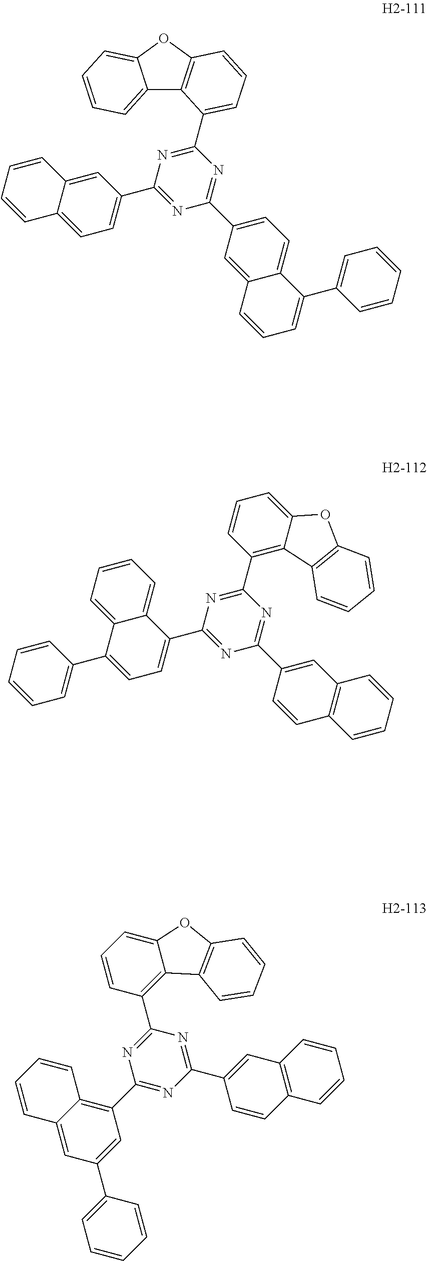 [SMILES: c1ccc(-c2cccc3cc(-c4nc(-c5ccc6ccccc6c5)nc(-c5cccc6oc7ccccc7c56)n4)ccc23)cc1 ; c1ccc(-c2ccc(-c3nc(-c4ccc5ccccc5c4)nc(-c4cccc5oc6ccccc6c45)n3)c3ccccc23)cc1 ; c1ccc(-c2cc(-c3nc(-c4ccc5ccccc5c4)nc(-c4cccc5oc6ccccc6c45)n3)c3ccccc3c2)cc1]

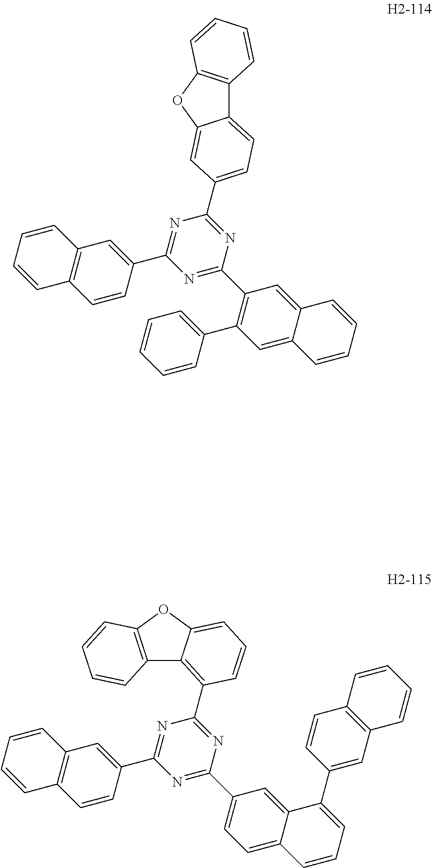 [SMILES: c1ccc(-c2cc3ccccc3cc2-c2nc(-c3ccc4ccccc4c3)nc(-c3ccc4c(c3)oc3ccccc34)n2)cc1 ; c1ccc2cc(-c3nc(-c4ccc5cccc(-c6ccc7ccccc7c6)c5c4)nc(-c4cccc5oc6ccccc6c45)n3)ccc2c1]

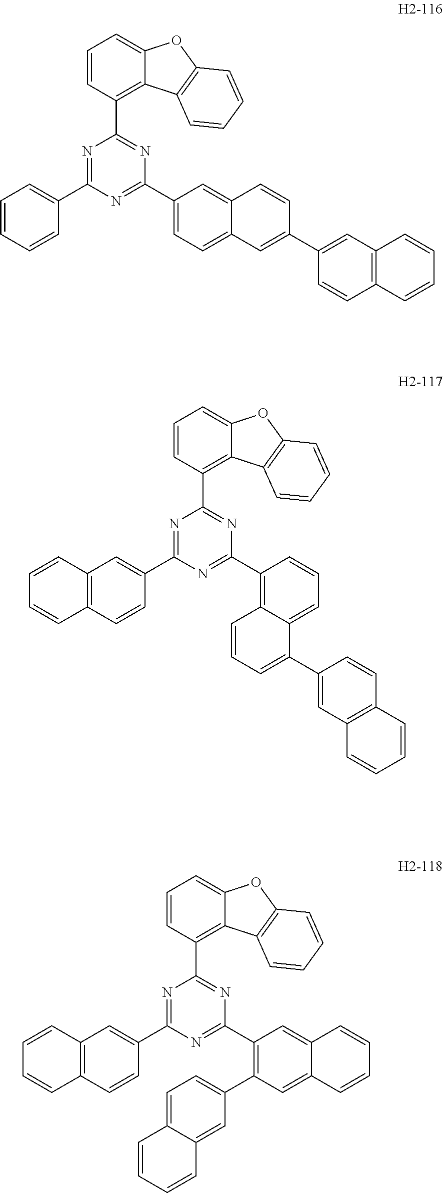 [SMILES: c1ccc(-c2nc(-c3ccc4cc(-c5ccc6ccccc6c5)ccc4c3)nc(-c3cccc4oc5ccccc5c34)n2)cc1 ; c1ccc2cc(-c3nc(-c4cccc5c(-c6ccc7ccccc7c6)cccc45)nc(-c4cccc5oc6ccccc6c45)n3)ccc2c1 ; c1ccc2cc(-c3nc(-c4cc5ccccc5cc4-c4ccc5ccccc5c4)nc(-c4cccc5oc6ccccc6c45)n3)ccc2c1]

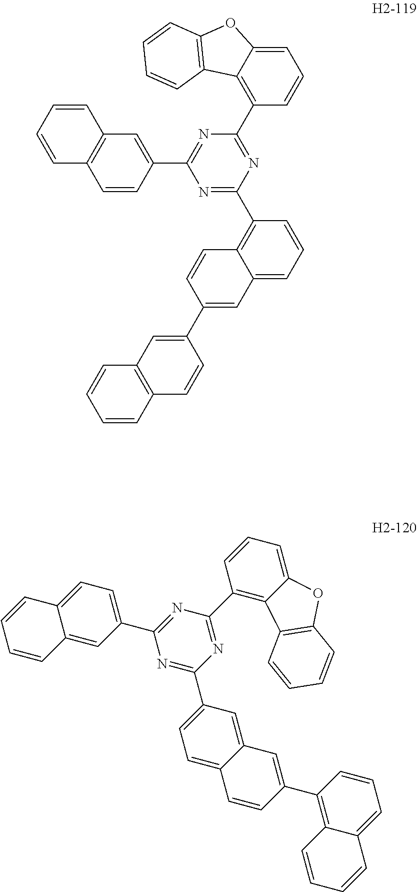 [SMILES: c1ccc2cc(-c3ccc4c(-c5nc(-c6ccc7ccccc7c6)nc(-c6cccc7oc8ccccc8c67)n5)cccc4c3)ccc2c1 ; c1ccc2cc(-c3nc(-c4ccc5ccc(-c6cccc7ccccc67)cc5c4)nc(-c4cccc5oc6ccccc6c45)n3)ccc2c1]

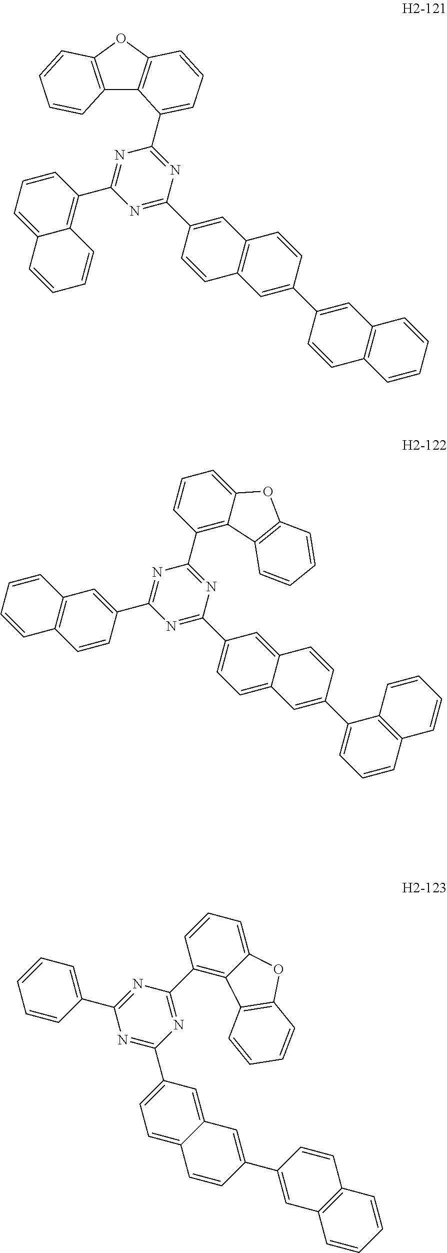 [SMILES: c1ccc2cc(-c3ccc4cc(-c5nc(-c6cccc7ccccc67)nc(-c6cccc7oc8ccccc8c67)n5)ccc4c3)ccc2c1 ; c1ccc2cc(-c3nc(-c4ccc5cc(-c6cccc7ccccc67)ccc5c4)nc(-c4cccc5oc6ccccc6c45)n3)ccc2c1 ; c1ccc(-c2nc(-c3ccc4ccc(-c5ccc6ccccc6c5)cc4c3)nc(-c3cccc4oc5ccccc5c34)n2)cc1]

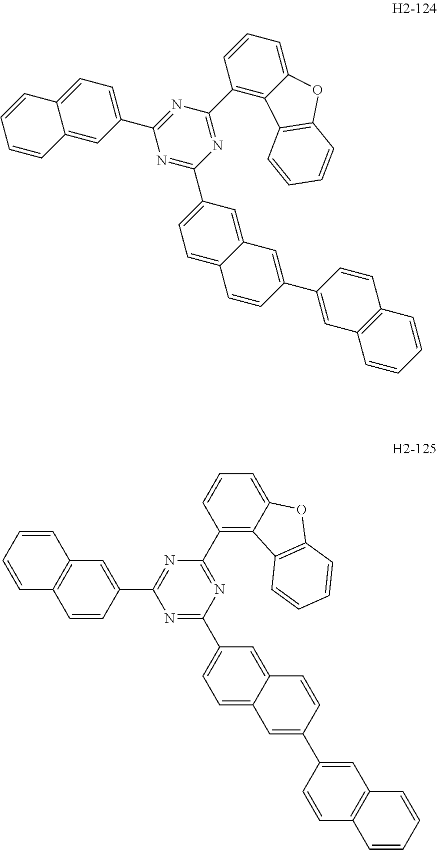 [SMILES: c1ccc2cc(-c3ccc4ccc(-c5nc(-c6ccc7ccccc7c6)nc(-c6cccc7oc8ccccc8c67)n5)cc4c3)ccc2c1 ; c1ccc2cc(-c3ccc4cc(-c5nc(-c6ccc7ccccc7c6)nc(-c6cccc7oc8ccccc8c67)n5)ccc4c3)ccc2c1]

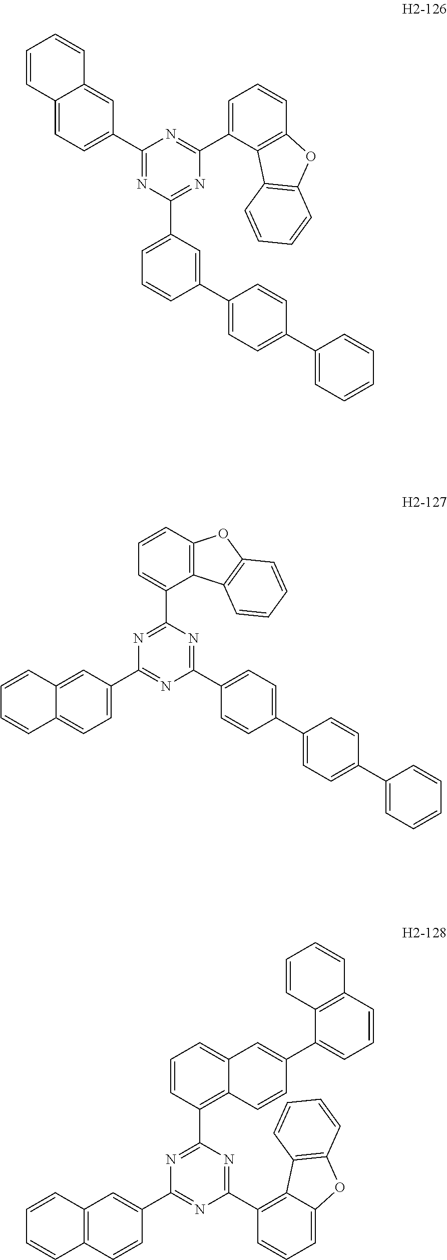 [SMILES: c1ccc(-c2ccc(-c3cccc(-c4nc(-c5ccc6ccccc6c5)nc(-c5cccc6oc7ccccc7c56)n4)c3)cc2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc2cc(-c3nc(-c4cccc5cc(-c6cccc7ccccc67)ccc45)nc(-c4cccc5oc6ccccc6c45)n3)ccc2c1]

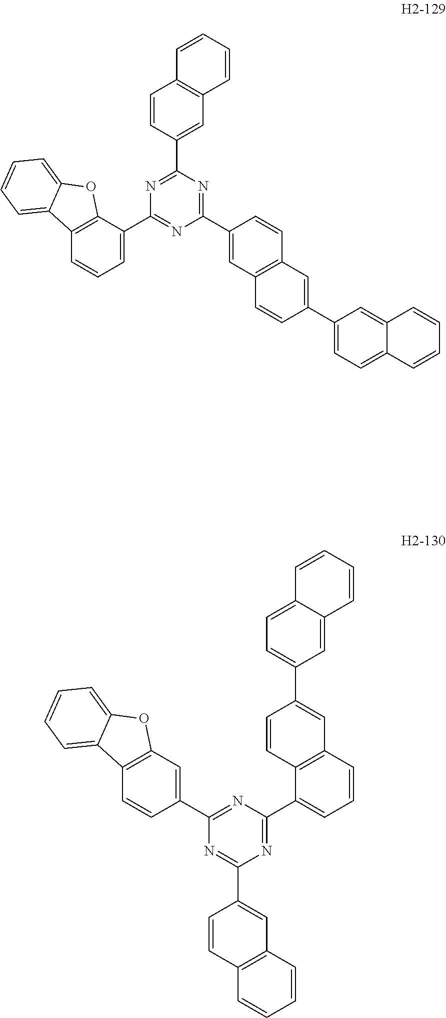 [SMILES: c1ccc2cc(-c3ccc4cc(-c5nc(-c6ccc7ccccc7c6)nc(-c6cccc7c6oc6ccccc67)n5)ccc4c3)ccc2c1 ; c1ccc2cc(-c3ccc4c(-c5nc(-c6ccc7ccccc7c6)nc(-c6ccc7c(c6)oc6ccccc67)n5)cccc4c3)ccc2c1]

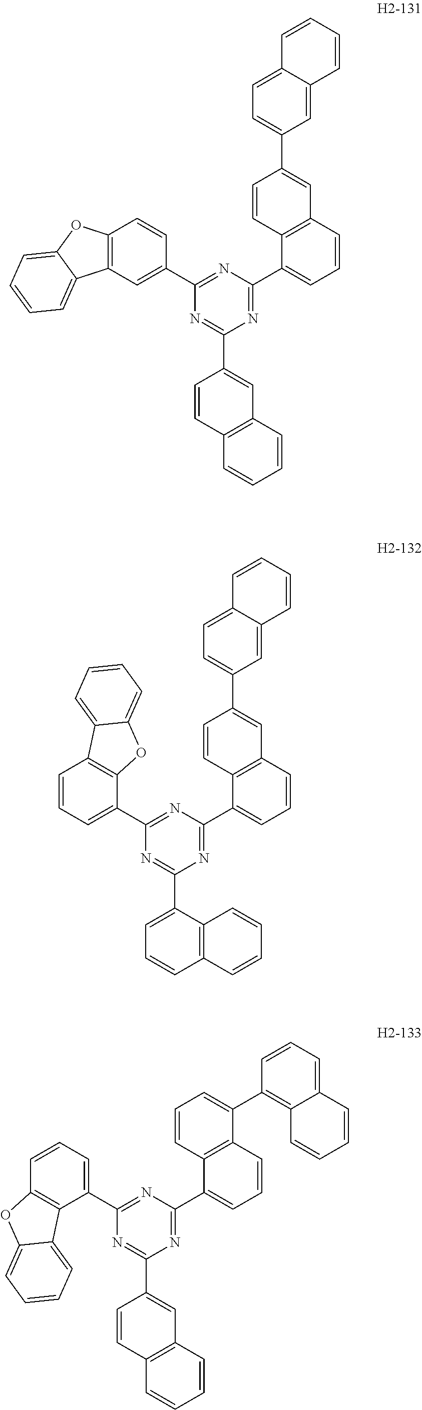 [SMILES: c1ccc2cc(-c3ccc4c(-c5nc(-c6ccc7ccccc7c6)nc(-c6ccc7oc8ccccc8c7c6)n5)cccc4c3)ccc2c1 ; c1ccc2cc(-c3ccc4c(-c5nc(-c6cccc7ccccc67)nc(-c6cccc7c6oc6ccccc67)n5)cccc4c3)ccc2c1 ; c1ccc2cc(-c3nc(-c4cccc5c(-c6cccc7ccccc67)cccc45)nc(-c4cccc5oc6ccccc6c45)n3)ccc2c1]

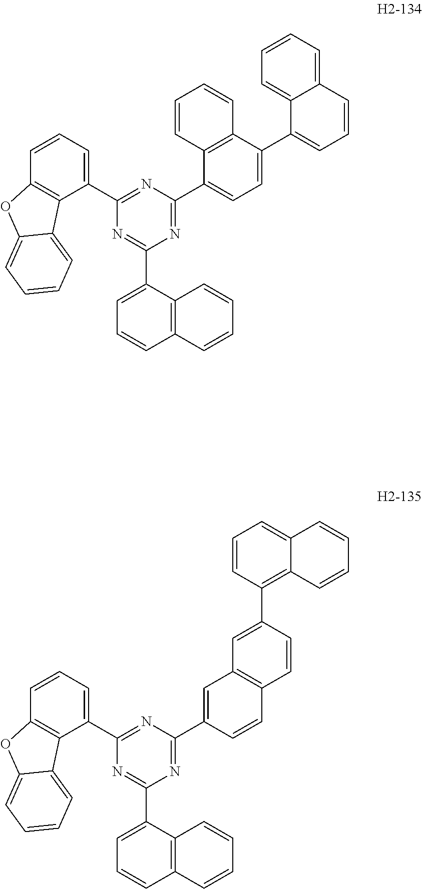 [SMILES: c1ccc2c(-c3nc(-c4ccc(-c5cccc6ccccc56)c5ccccc45)nc(-c4cccc5oc6ccccc6c45)n3)cccc2c1 ; c1ccc2c(-c3ccc4ccc(-c5nc(-c6cccc7ccccc67)nc(-c6cccc7oc8ccccc8c67)n5)cc4c3)cccc2c1]

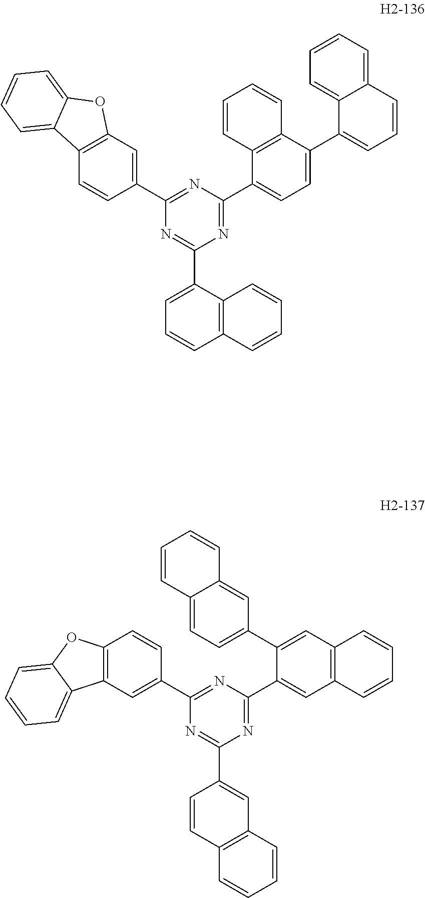 [SMILES: c1ccc2c(-c3nc(-c4ccc5c(c4)oc4ccccc45)nc(-c4ccc(-c5cccc6ccccc56)c5ccccc45)n3)cccc2c1 ; c1ccc2cc(-c3nc(-c4ccc5oc6ccccc6c5c4)nc(-c4cc5ccccc5cc4-c4ccc5ccccc5c4)n3)ccc2c1]

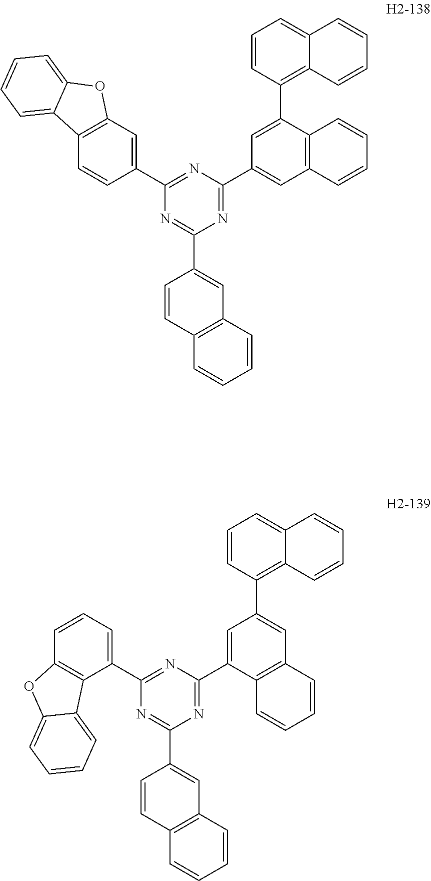 [SMILES: c1ccc2cc(-c3nc(-c4cc(-c5cccc6ccccc56)c5ccccc5c4)nc(-c4ccc5c(c4)oc4ccccc45)n3)ccc2c1 ; c1ccc2cc(-c3nc(-c4cc(-c5cccc6ccccc56)cc5ccccc45)nc(-c4cccc5oc6ccccc6c45)n3)ccc2c1]

A combination of at least one of the compounds H1-1 to H1-300, and at least one of compounds H2-1 to H2-139, may be used in an organic electroluminescent device.

Hereinafter, an organic electroluminescent compound represented by the following formula 1 will be described in more detail.

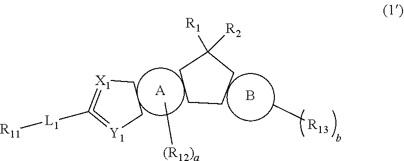

In formula 1,
R₁ and R₂ each independently represent a substituted or unsubstituted (C1-C30)alkyl, a substituted or unsubstituted (C6-C30)aryl, or a substituted or unsubstituted (3- to 30-membered)heteroaryl; or may be linked to each other to form a ring(s). For example, R₁ and R₂ each independently may be a methyl, etc.
X₁ and Y₁ each independently represent —N═, —NR₁₄, —O— or —S—, with a proviso that any one of X₁ and Y₁ represents —N═, and the other of X₁ and Y₁ represents —NR₁₄—, —O— or —S—. For example, X₁ may be —N═, Y₁ may be —NR₁₄, —O— or —S—, etc.
Ring A and ring B each independently represent a substituted or unsubstituted (C6-C30)arene, or a substituted or unsubstituted (3- to 30-membered)heteroarene. According to one embodiment of the present disclosure, ring A and ring B each independently may be a substituted or unsubstituted (C6-C18)arene. According to one embodiment of the present disclosure, ring A and ring B each independently may be an unsubstituted (C6-C18)arene. For example, ring A and ring B each independently may be a benzene, a naphthalene, a phenanthrene, etc.
R₁₁ and R₁₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or the formula 1′-1 or 1′-2, with a proviso that at least one of R₁₁ to R₁₄ is represented by the following formula 1′-1 or 1′-2, where if R₁₁ is represented by the following formula 1′-1 or 1′-2, and R₁₁ is represented by the formula 1′-2. According to one embodiment of the present disclosure, R₁₁ to R₁₄ each independently may be hydrogen, a substituted or unsubstituted (C6-C25)aryl, formula 1′-1, formula 1′-2. For example, R₁₁ to R₁₄ each independently may be hydrogen, a naphthyl, a phenyl unsubstituted or substituted with a methyl(s) or a ethylenyl(s), formula 1′-1, formula 1′-2, etc.

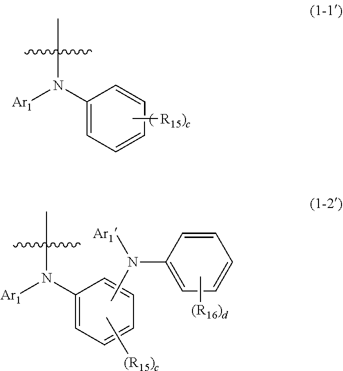

R₁₅ and R₁₆ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₂-N(Ar₂)(Ar₃). According to one embodiment of the present disclosure, R₁₅ and R₁₆ each independently may be hydrogen, a substituted or unsubstituted (C6-C18)aryl, or -L₂-N(Ar₂)(Ar₃). For example, R₁₅ and R₁₆ each independently may be hydrogen, a phenyl, -L₂-N(Ar₂)(Ar₃), etc.
L₁ and L₂ each independently represent a single bond, a substituted or unsubstituted (C6-C30)arylene, or a substituted or unsubstituted (3- to 30-membered)heteroarylene. According to one embodiment of the present disclosure, L₁ and L₂ each independently may be a single bond, or a substituted or unsubstituted (C6-C18)arylene. For example, L₁ and L₂ each independently may be a single bond, a phenylene, etc.
Ar₁ to Ar₃ and Ar₁′ each independently represent hydrogen, deuterium, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl. According to one embodiment of the present disclosure, Ar₁ to Ar₃ and Ar₁′ each independently may be a substituted or unsubstituted (C6-C25)aryl, or a substituted or unsubstituted (3- to 25-membered)heteroaryl. For example, Ar₁ to Ar₃ and Ar₁′ each independently may be a phenyl, a dibenzofuranyl, a carbazolyl substituted with a phenyl(s), a dibenzothiophenyl, a naphthyl, etc.
a represents an integer of 1 to 6, b represents an integer of 1 to 8, c′ represents an integer of 1 to 4, c and d represent an integer of 1 to 5, where if a to d and c′ are an integer of 2 or more, each of R₁₂, each of R₁₃, each of R₁₅, and each of R₁₆ may be the same as, or different from, each other.

According to one embodiment of the present disclosure, the above formula 1′ is an organic electroluminescent compound represented by one of the following formulas 3′ and 4′.

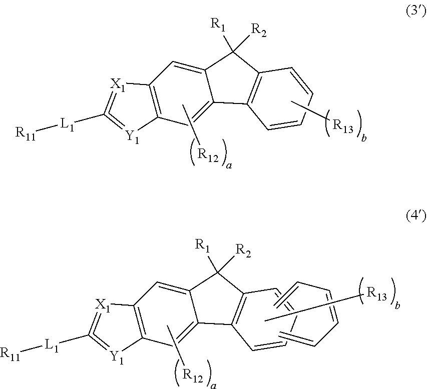

in the formulas 3′ and 4′,
R₁, R₂, R₁₁ to R₁₃, X₁, Y₁, and L₁ are as defined in formula 1′, a represents an integer of 1 or 2, and b represents an integer of 1 to 6.

According to one embodiment of the present disclosure, the compound represented by formula 1′ may be an organic electroluminescent compound selected from the following compounds.

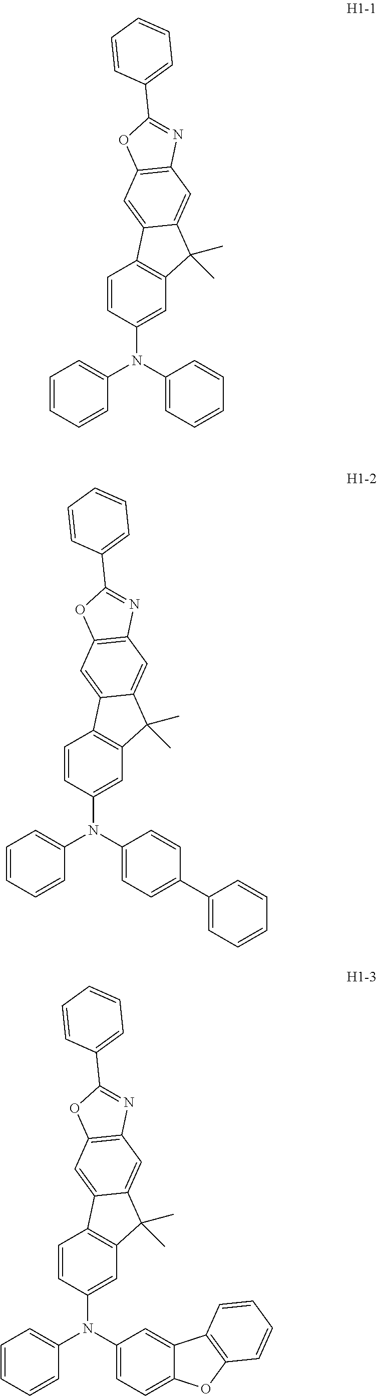 [SMILES: CC1(C)c2cc(N(c3ccccc3)c3ccccc3)ccc2-c2cc3oc(-c4ccccc4)nc3cc21 ; CC1(C)c2cc(N(c3ccccc3)c3ccc(-c4ccccc4)cc3)ccc2-c2cc3oc(-c4ccccc4)nc3cc21 ; CC1(C)c2cc(N(c3ccccc3)c3ccc4oc5ccccc5c4c3)ccc2-c2cc3oc(-c4ccccc4)nc3cc21]

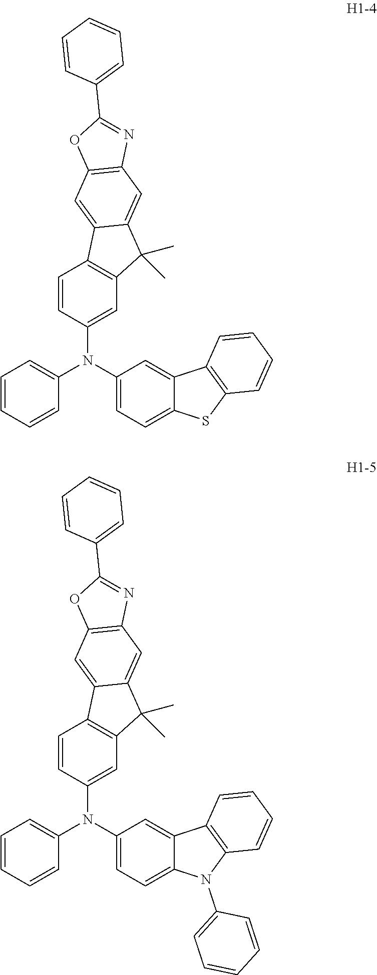 [SMILES: CC1(C)c2cc(N(c3ccccc3)c3ccc4sc5ccccc5c4c3)ccc2-c2cc3oc(-c4ccccc4)nc3cc21 ; CC1(C)c2cc(N(c3ccccc3)c3ccc4c(c3)c3ccccc3n4-c3ccccc3)ccc2-c2cc3oc(-c4ccccc4)nc3cc21]

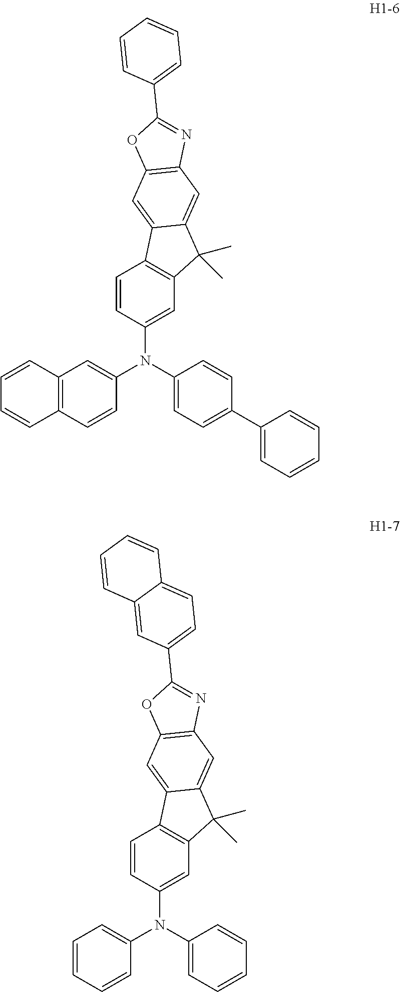 [SMILES: CC1(C)c2cc(N(c3ccc(-c4ccccc4)cc3)c3ccc4ccccc4c3)ccc2-c2cc3oc(-c4ccccc4)nc3cc21 ; CC1(C)c2cc(N(c3ccccc3)c3ccccc3)ccc2-c2cc3oc(-c4ccc5ccccc5c4)nc3cc21]

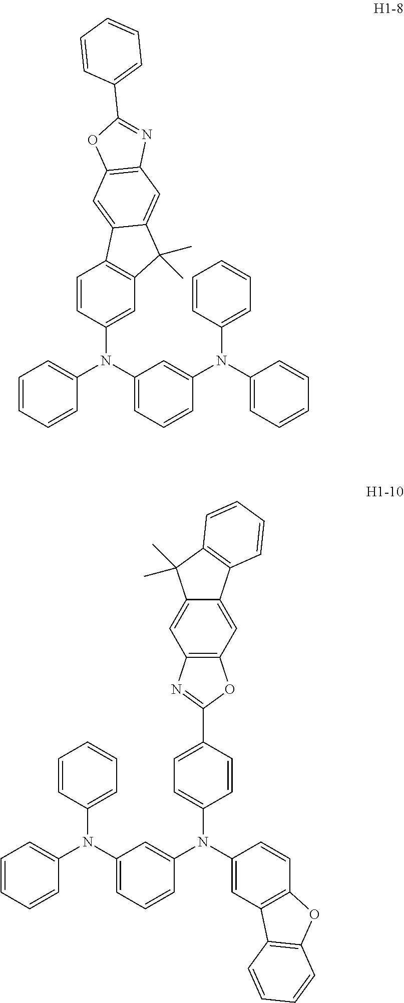 [SMILES: CC1(C)c2cc(N(c3ccccc3)c3cccc(N(c4ccccc4)c4ccccc4)c3)ccc2-c2cc3oc(-c4ccccc4)nc3cc21 ; CC1(C)c2ccccc2-c2cc3oc(-c4ccc(N(c5cccc(N(c6ccccc6)c6ccccc6)c5)c5ccc6oc7ccccc7c6c5)cc4)nc3cc21]

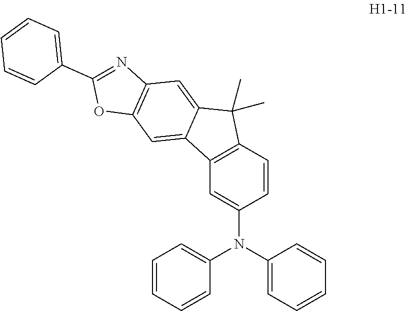 [SMILES: CC1(C)c2ccc(N(c3ccccc3)c3ccccc3)cc2-c2cc3oc(-c4ccccc4)nc3cc21]

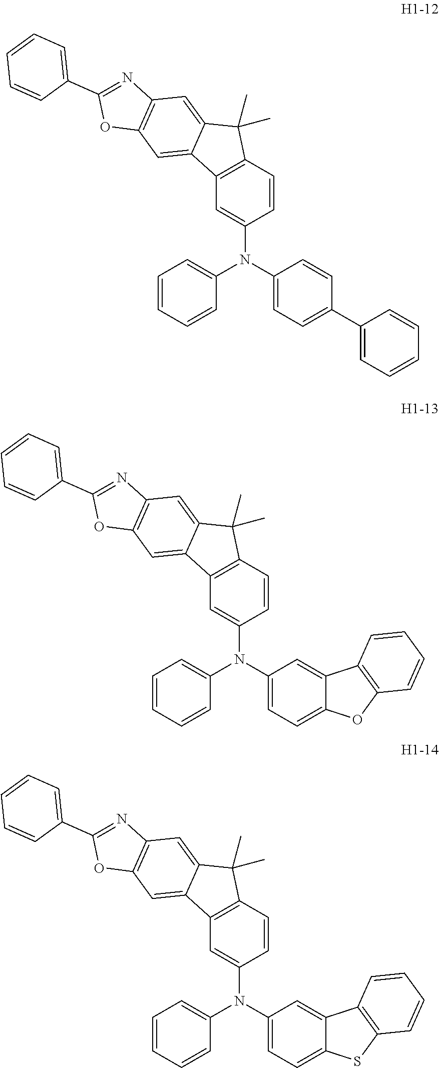 [SMILES: CC1(C)c2ccc(N(c3ccccc3)c3ccc(-c4ccccc4)cc3)cc2-c2cc3oc(-c4ccccc4)nc3cc21 ; CC1(C)c2ccc(N(c3ccccc3)c3ccc4oc5ccccc5c4c3)cc2-c2cc3oc(-c4ccccc4)nc3cc21 ; CC1(C)c2ccc(N(c3ccccc3)c3ccc4sc5ccccc5c4c3)cc2-c2cc3oc(-c4ccccc4)nc3cc21]

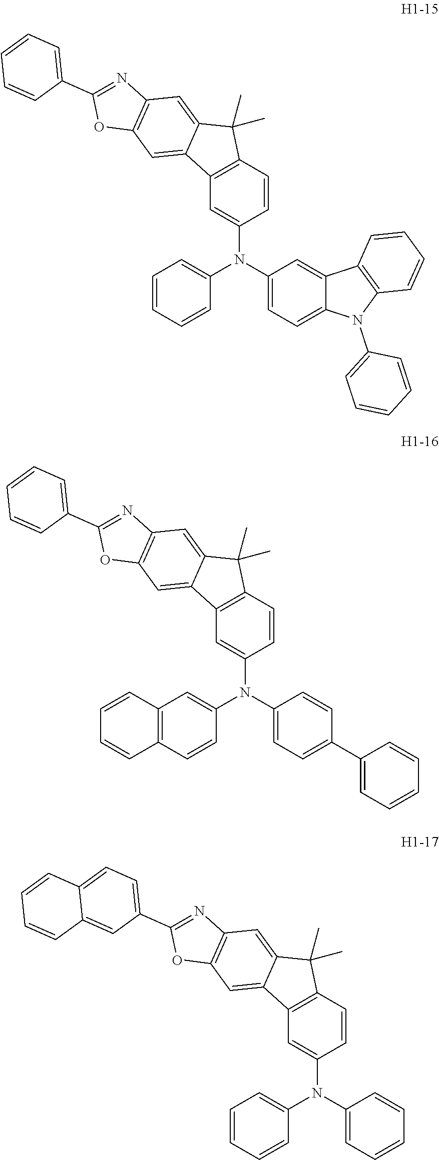 [SMILES: CC1(C)c2ccc(N(c3ccccc3)c3ccc4c(c3)c3ccccc3n4-c3ccccc3)cc2-c2cc3oc(-c4ccccc4)nc3cc21 ; CC1(C)c2ccc(N(c3ccc(-c4ccccc4)cc3)c3ccc4ccccc4c3)cc2-c2cc3oc(-c4ccccc4)nc3cc21 ; CC1(C)c2ccc(N(c3ccccc3)c3ccccc3)cc2-c2cc3oc(-c4ccc5ccccc5c4)nc3cc21]

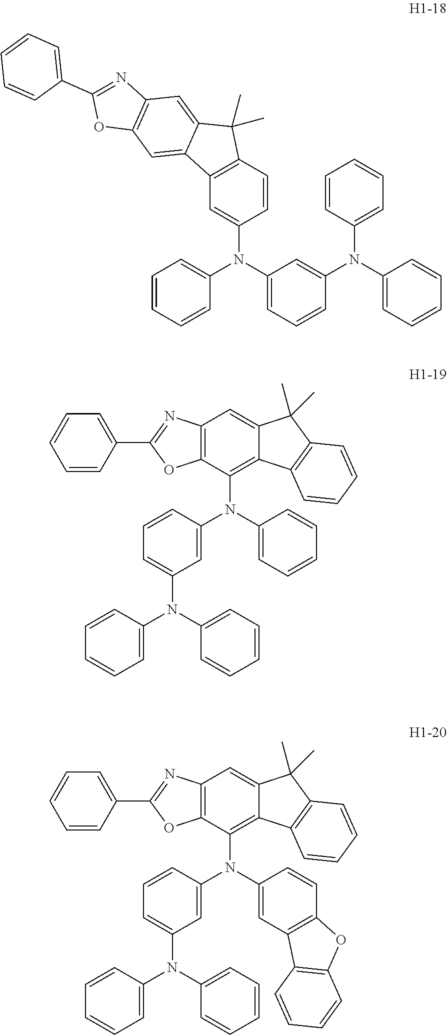 [SMILES: CC1(C)c2ccc(N(c3ccccc3)c3cccc(N(c4ccccc4)c4ccccc4)c3)cc2-c2cc3oc(-c4ccccc4)nc3cc21 ; CC1(C)c2ccccc2-c2c1cc1nc(-c3ccccc3)oc1c2N(c1ccccc1)c1cccc(N(c2ccccc2)c2ccccc2)c1 ; CC1(C)c2ccccc2-c2c1cc1nc(-c3ccccc3)oc1c2N(c1cccc(N(c2ccccc2)c2ccccc2)c1)c1ccc2oc3ccccc3c2c1]

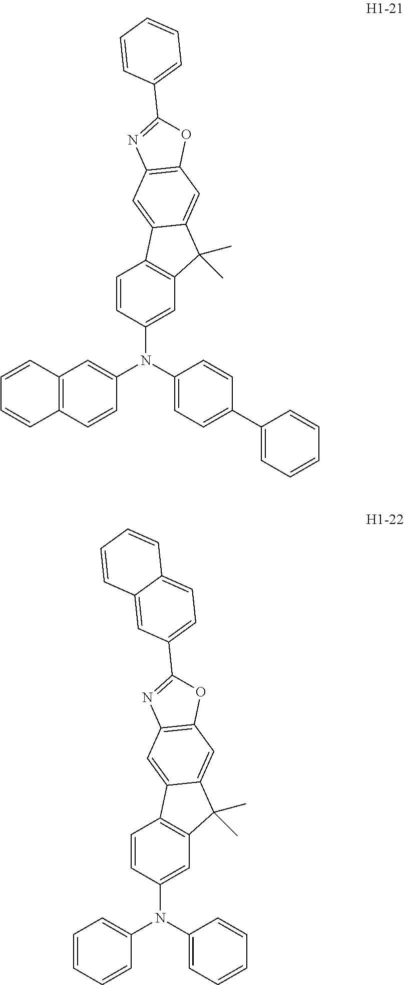 [SMILES: CC1(C)c2cc(N(c3ccc(-c4ccccc4)cc3)c3ccc4ccccc4c3)ccc2-c2cc3nc(-c4ccccc4)oc3cc21 ; CC1(C)c2cc(N(c3ccccc3)c3ccccc3)ccc2-c2cc3nc(-c4ccc5ccccc5c4)oc3cc21]

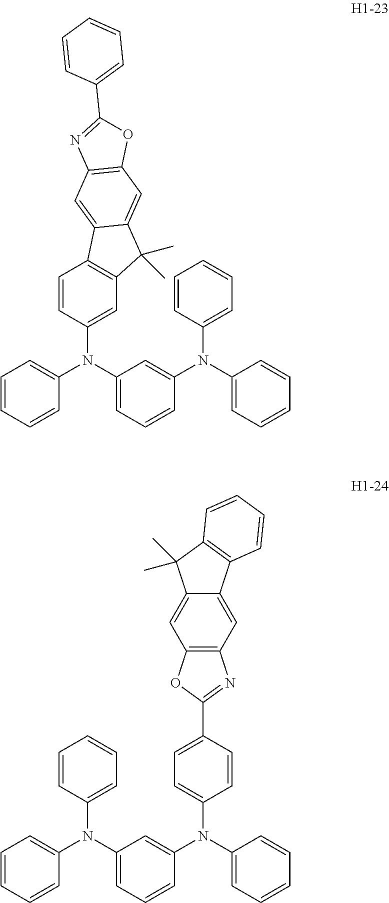 [SMILES: CC1(C)c2cc(N(c3ccccc3)c3cccc(N(c4ccccc4)c4ccccc4)c3)ccc2-c2cc3nc(-c4ccccc4)oc3cc21 ; CC1(C)c2ccccc2-c2cc3nc(-c4ccc(N(c5ccccc5)c5cccc(N(c6ccccc6)c6ccccc6)c5)cc4)oc3cc21]

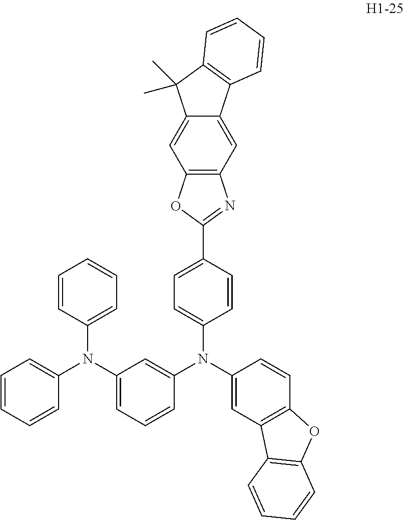 [SMILES: CC1(C)c2ccccc2-c2cc3nc(-c4ccc(N(c5cccc(N(c6ccccc6)c6ccccc6)c5)c5ccc6oc7ccccc7c6c5)cc4)oc3cc21]

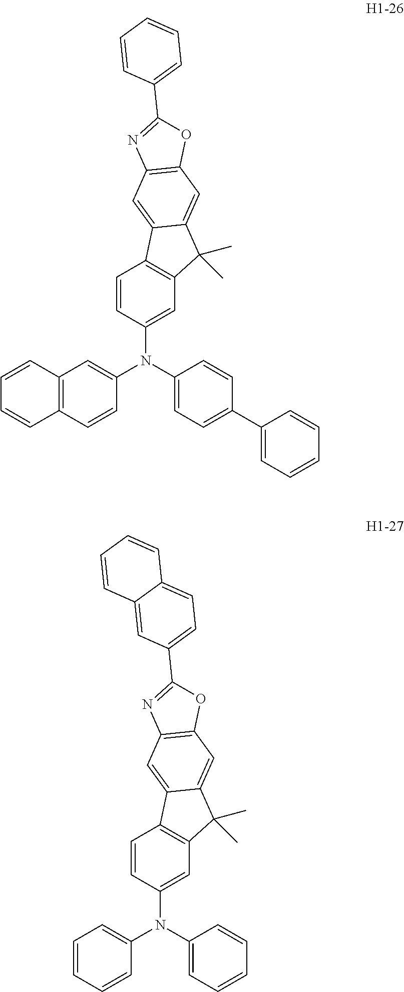 [SMILES: CC1(C)c2cc(N(c3ccc(-c4ccccc4)cc3)c3ccc4ccccc4c3)ccc2-c2cc3nc(-c4ccccc4)oc3cc21 ; CC1(C)c2cc(N(c3ccccc3)c3ccccc3)ccc2-c2cc3nc(-c4ccc5ccccc5c4)oc3cc21]

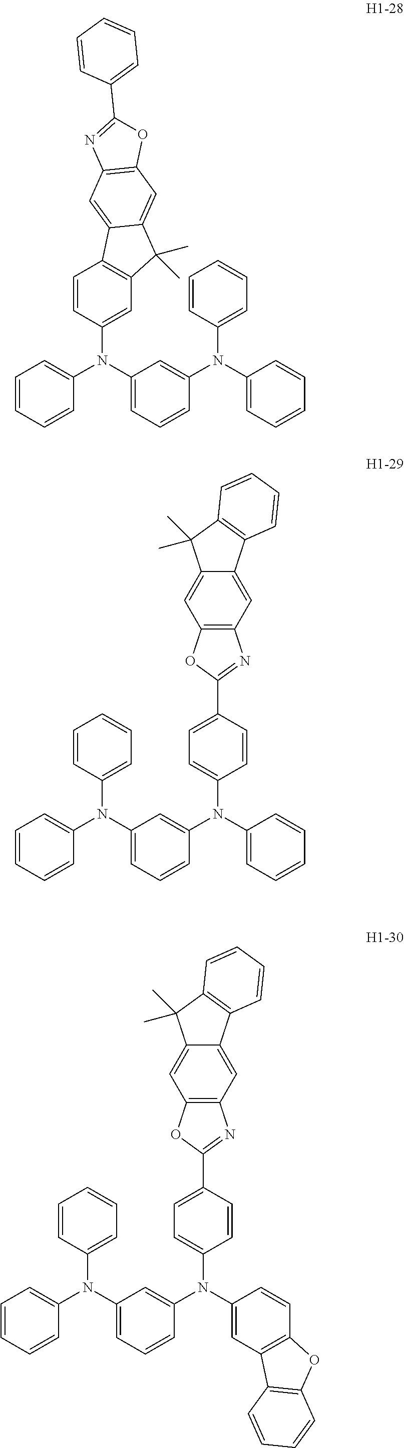 [SMILES: CC1(C)c2cc(N(c3ccccc3)c3cccc(N(c4ccccc4)c4ccccc4)c3)ccc2-c2cc3nc(-c4ccccc4)oc3cc21 ; CC1(C)c2ccccc2-c2cc3nc(-c4ccc(N(c5ccccc5)c5cccc(N(c6ccccc6)c6ccccc6)c5)cc4)oc3cc21 ; CC1(C)c2ccccc2-c2cc3nc(-c4ccc(N(c5cccc(N(c6ccccc6)c6ccccc6)c5)c5ccc6oc7ccccc7c6c5)cc4)oc3cc21]

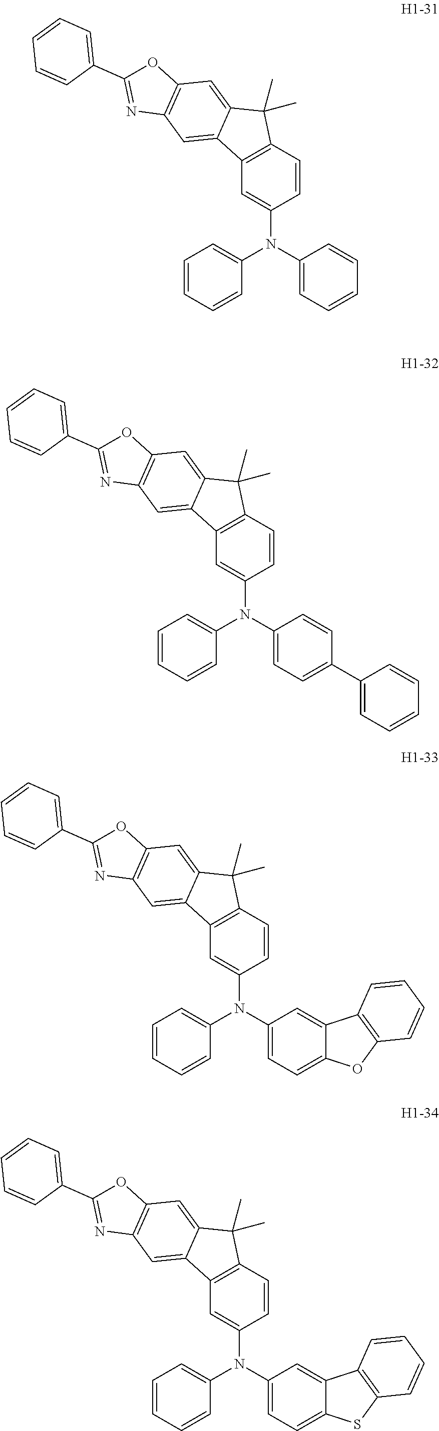 [SMILES: CC1(C)c2ccc(N(c3ccccc3)c3ccccc3)cc2-c2cc3nc(-c4ccccc4)oc3cc21 ; CC1(C)c2ccc(N(c3ccccc3)c3ccc(-c4ccccc4)cc3)cc2-c2cc3nc(-c4ccccc4)oc3cc21 ; CC1(C)c2ccc(N(c3ccccc3)c3ccc4oc5ccccc5c4c3)cc2-c2cc3nc(-c4ccccc4)oc3cc21 ; CC1(C)c2ccc(N(c3ccccc3)c3ccc4sc5ccccc5c4c3)cc2-c2cc3nc(-c4ccccc4)oc3cc21]

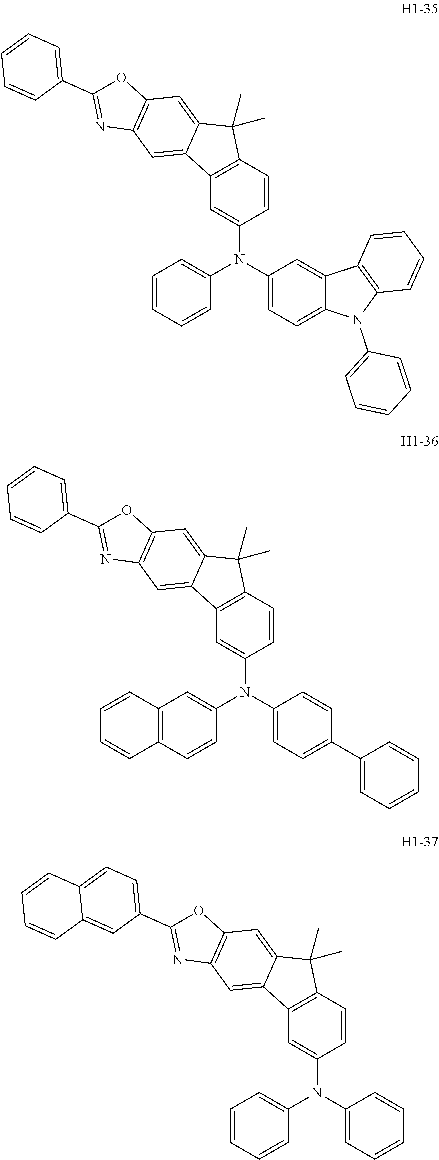 [SMILES: CC1(C)c2ccc(N(c3ccccc3)c3ccc4c(c3)c3ccccc3n4-c3ccccc3)cc2-c2cc3nc(-c4ccccc4)oc3cc21 ; CC1(C)c2ccc(N(c3ccc(-c4ccccc4)cc3)c3ccc4ccccc4c3)cc2-c2cc3nc(-c4ccccc4)oc3cc21 ; CC1(C)c2ccc(N(c3ccccc3)c3ccccc3)cc2-c2cc3nc(-c4ccc5ccccc5c4)oc3cc21]

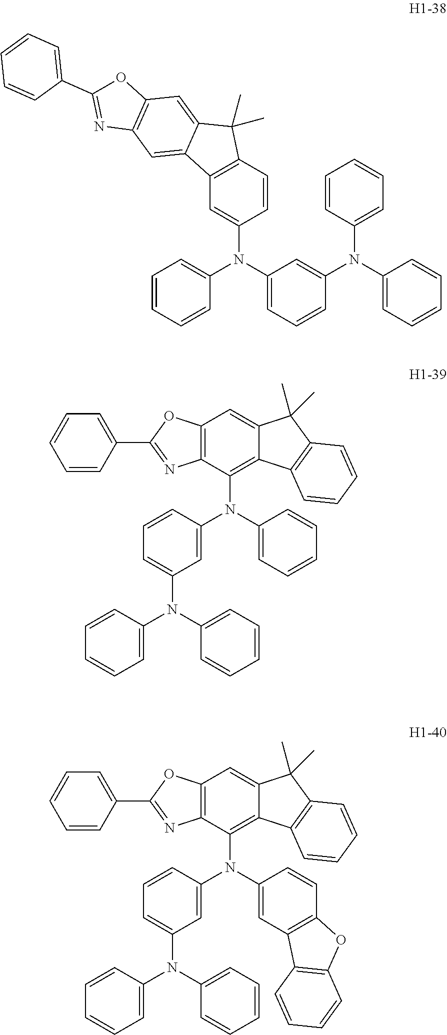 [SMILES: CC1(C)c2ccc(N(c3ccccc3)c3cccc(N(c4ccccc4)c4ccccc4)c3)cc2-c2cc3nc(-c4ccccc4)oc3cc21 ; CC1(C)c2ccccc2-c2c1cc1oc(-c3ccccc3)nc1c2N(c1ccccc1)c1cccc(N(c2ccccc2)c2ccccc2)c1 ; CC1(C)c2ccccc2-c2c1cc1oc(-c3ccccc3)nc1c2N(c1cccc(N(c2ccccc2)c2ccccc2)c1)c1ccc2oc3ccccc3c2c1]

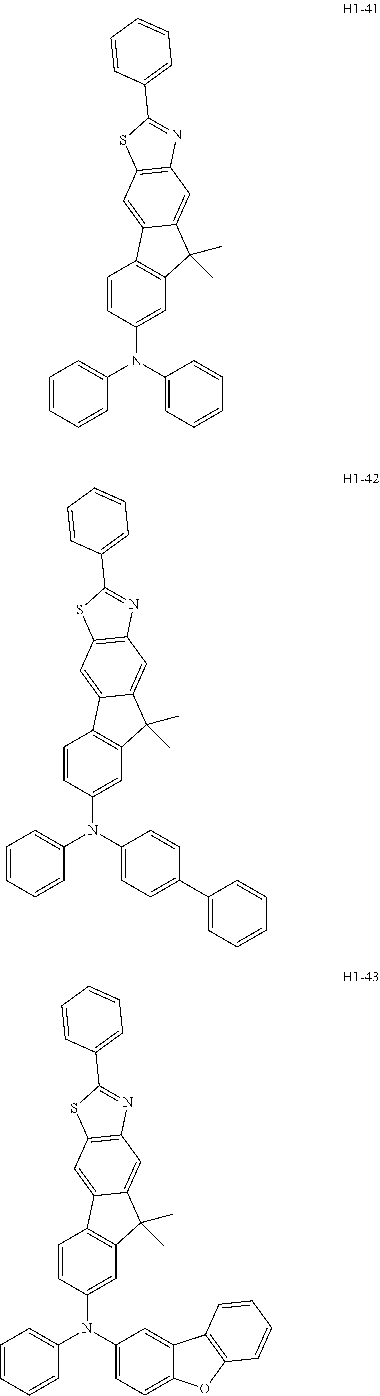 [SMILES: CC1(C)c2cc(N(c3ccccc3)c3ccccc3)ccc2-c2cc3sc(-c4ccccc4)nc3cc21 ; CC1(C)c2cc(N(c3ccccc3)c3ccc(-c4ccccc4)cc3)ccc2-c2cc3sc(-c4ccccc4)nc3cc21 ; CC1(C)c2cc(N(c3ccccc3)c3ccc4oc5ccccc5c4c3)ccc2-c2cc3sc(-c4ccccc4)nc3cc21]

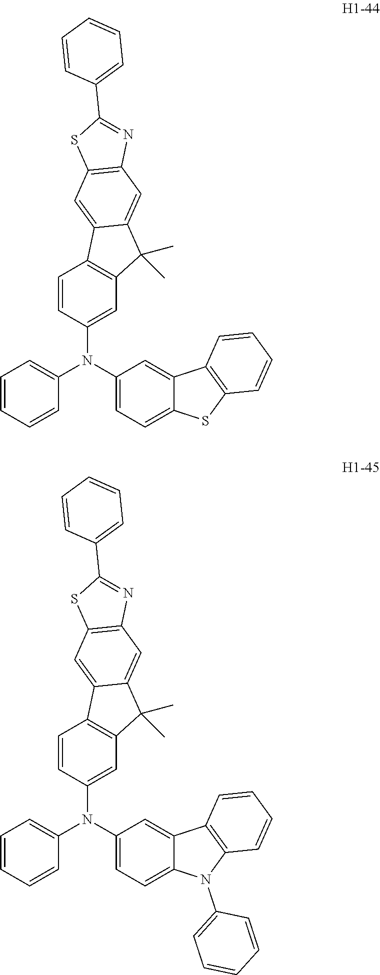 [SMILES: CC1(C)c2cc(N(c3ccccc3)c3ccc4sc5ccccc5c4c3)ccc2-c2cc3sc(-c4ccccc4)nc3cc21 ; CC1(C)c2cc(N(c3ccccc3)c3ccc4c(c3)c3ccccc3n4-c3ccccc3)ccc2-c2cc3sc(-c4ccccc4)nc3cc21]

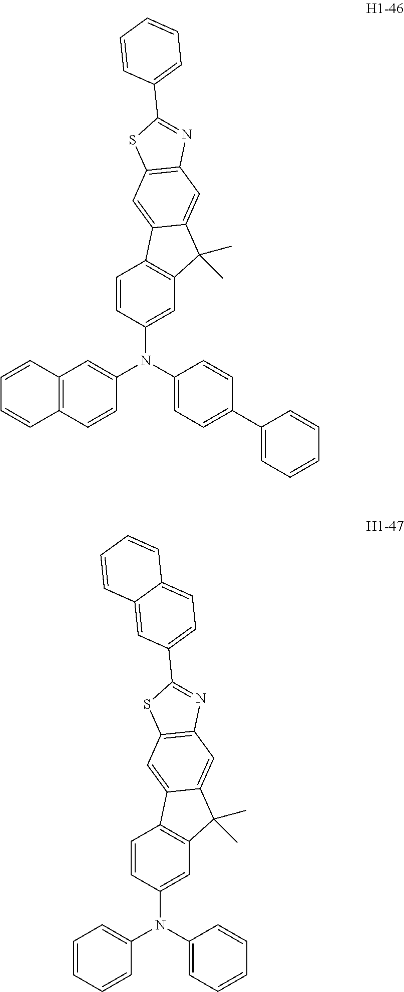 [SMILES: CC1(C)c2cc(N(c3ccc(-c4ccccc4)cc3)c3ccc4ccccc4c3)ccc2-c2cc3sc(-c4ccccc4)nc3cc21 ; CC1(C)c2cc(N(c3ccccc3)c3ccccc3)ccc2-c2cc3sc(-c4ccc5ccccc5c4)nc3cc21]

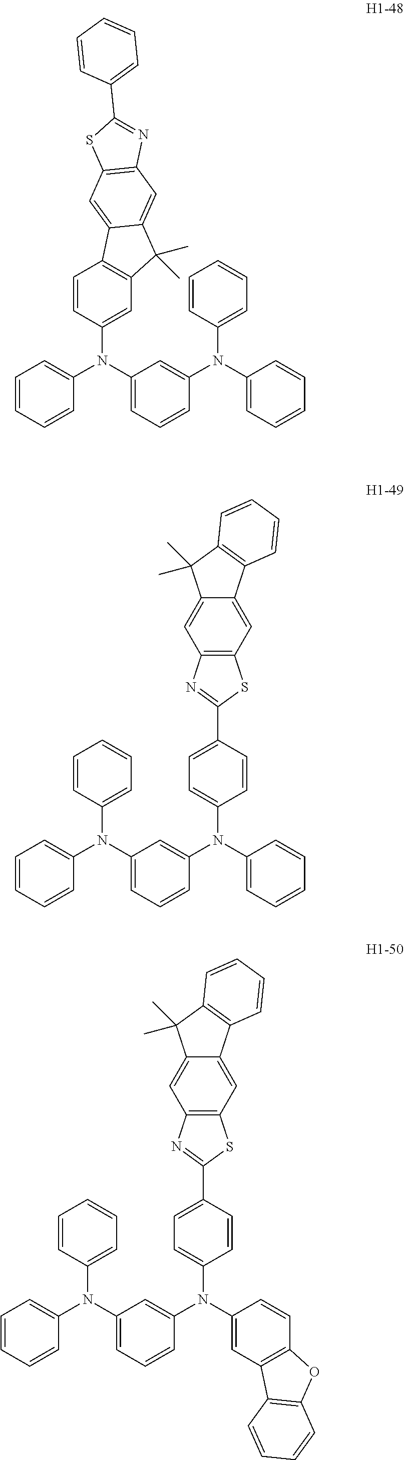 [SMILES: CC1(C)c2cc(N(c3ccccc3)c3cccc(N(c4ccccc4)c4ccccc4)c3)ccc2-c2cc3sc(-c4ccccc4)nc3cc21 ; CC1(C)c2ccccc2-c2cc3sc(-c4ccc(N(c5ccccc5)c5cccc(N(c6ccccc6)c6ccccc6)c5)cc4)nc3cc21 ; CC1(C)c2ccccc2-c2cc3sc(-c4ccc(N(c5cccc(N(c6ccccc6)c6ccccc6)c5)c5ccc6oc7ccccc7c6c5)cc4)nc3cc21]

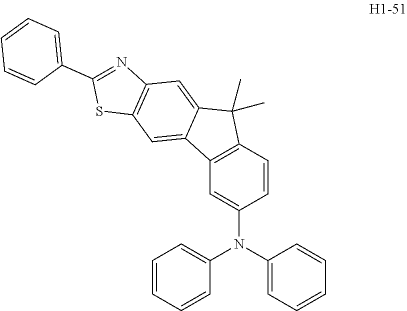 [SMILES: CC1(C)c2ccc(N(c3ccccc3)c3ccccc3)cc2-c2cc3sc(-c4ccccc4)nc3cc21]

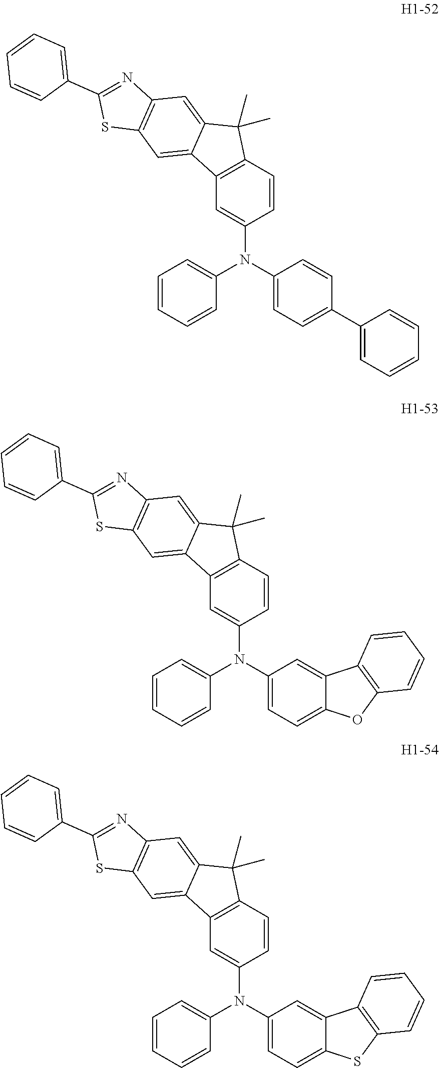 [SMILES: CC1(C)c2ccc(N(c3ccccc3)c3ccc(-c4ccccc4)cc3)cc2-c2cc3sc(-c4ccccc4)nc3cc21 ; CC1(C)c2ccc(N(c3ccccc3)c3ccc4oc5ccccc5c4c3)cc2-c2cc3sc(-c4ccccc4)nc3cc21 ; CC1(C)c2ccc(N(c3ccccc3)c3ccc4sc5ccccc5c4c3)cc2-c2cc3sc(-c4ccccc4)nc3cc21]

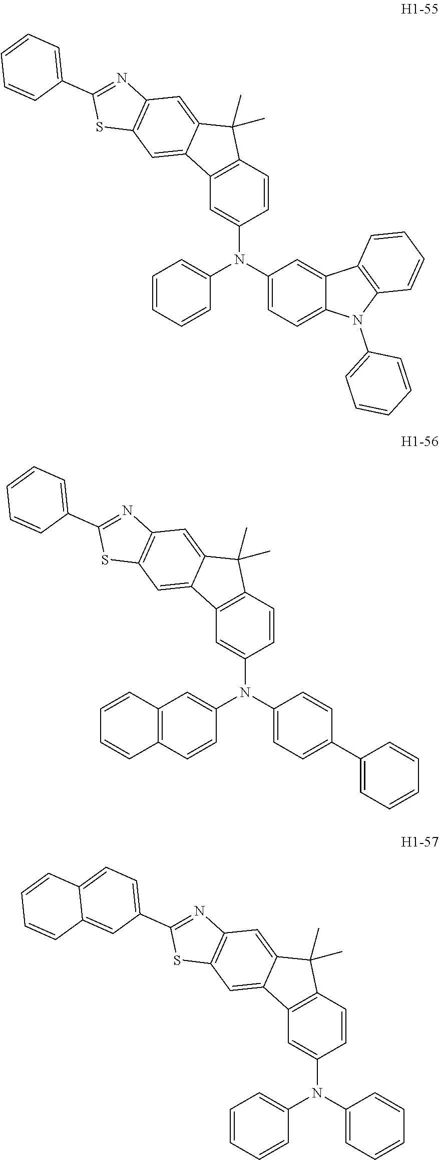 [SMILES: CC1(C)c2ccc(N(c3ccccc3)c3ccc4c(c3)c3ccccc3n4-c3ccccc3)cc2-c2cc3sc(-c4ccccc4)nc3cc21 ; CC1(C)c2ccc(N(c3ccc(-c4ccccc4)cc3)c3ccc4ccccc4c3)cc2-c2cc3sc(-c4ccccc4)nc3cc21 ; CC1(C)c2ccc(N(c3ccccc3)c3ccccc3)cc2-c2cc3sc(-c4ccc5ccccc5c4)nc3cc21]

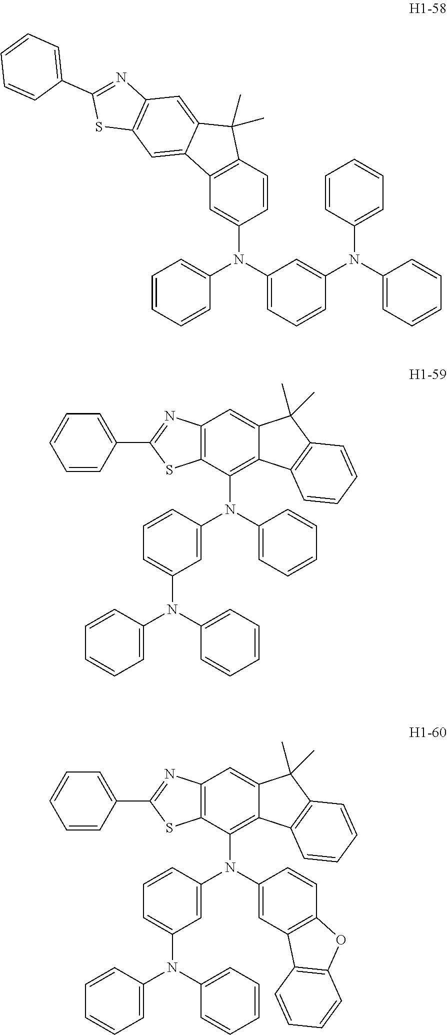 [SMILES: CC1(C)c2ccc(N(c3ccccc3)c3cccc(N(c4ccccc4)c4ccccc4)c3)cc2-c2cc3sc(-c4ccccc4)nc3cc21 ; CC1(C)c2ccccc2-c2c1cc1nc(-c3ccccc3)sc1c2N(c1ccccc1)c1cccc(N(c2ccccc2)c2ccccc2)c1 ; CC1(C)c2ccccc2-c2c1cc1nc(-c3ccccc3)sc1c2N(c1cccc(N(c2ccccc2)c2ccccc2)c1)c1ccc2oc3ccccc3c2c1]

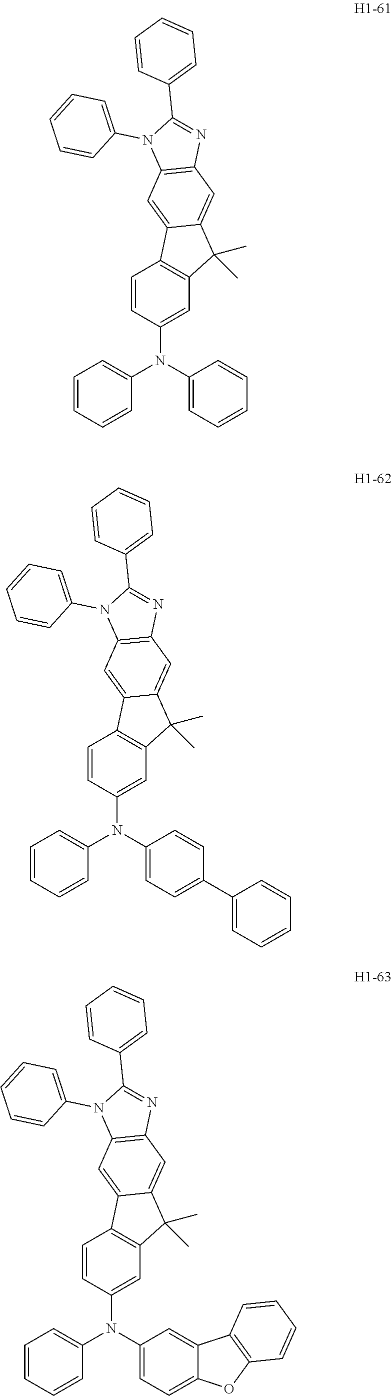 [SMILES: CC1(C)c2cc(N(c3ccccc3)c3ccccc3)ccc2-c2cc3c(cc21)nc(-c1ccccc1)n3-c1ccccc1 ; CC1(C)c2cc(N(c3ccccc3)c3ccc(-c4ccccc4)cc3)ccc2-c2cc3c(cc21)nc(-c1ccccc1)n3-c1ccccc1 ; CC1(C)c2cc(N(c3ccccc3)c3ccc4oc5ccccc5c4c3)ccc2-c2cc3c(cc21)nc(-c1ccccc1)n3-c1ccccc1]

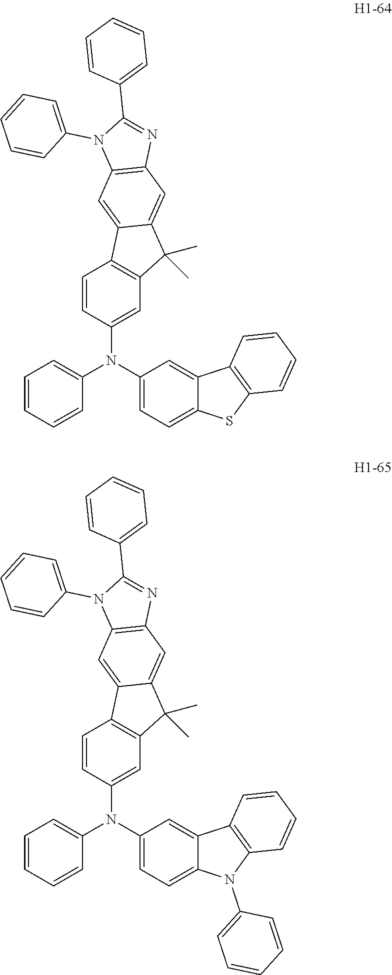 [SMILES: CC1(C)c2cc(N(c3ccccc3)c3ccc4sc5ccccc5c4c3)ccc2-c2cc3c(cc21)nc(-c1ccccc1)n3-c1ccccc1 ; CC1(C)c2cc(N(c3ccccc3)c3ccc4c(c3)c3ccccc3n4-c3ccccc3)ccc2-c2cc3c(cc21)nc(-c1ccccc1)n3-c1ccccc1]

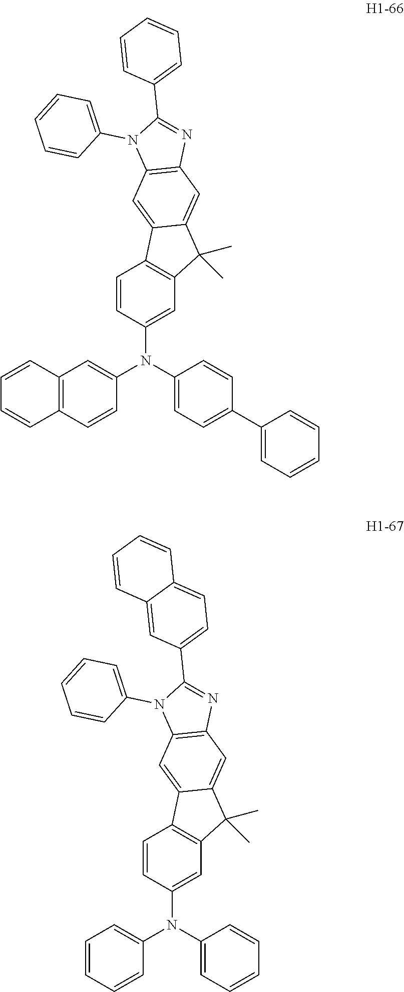 [SMILES: CC1(C)c2cc(N(c3ccc(-c4ccccc4)cc3)c3ccc4ccccc4c3)ccc2-c2cc3c(cc21)nc(-c1ccccc1)n3-c1ccccc1 ; CC1(C)c2cc(N(c3ccccc3)c3ccccc3)ccc2-c2cc3c(cc21)nc(-c1ccc2ccccc2c1)n3-c1ccccc1]

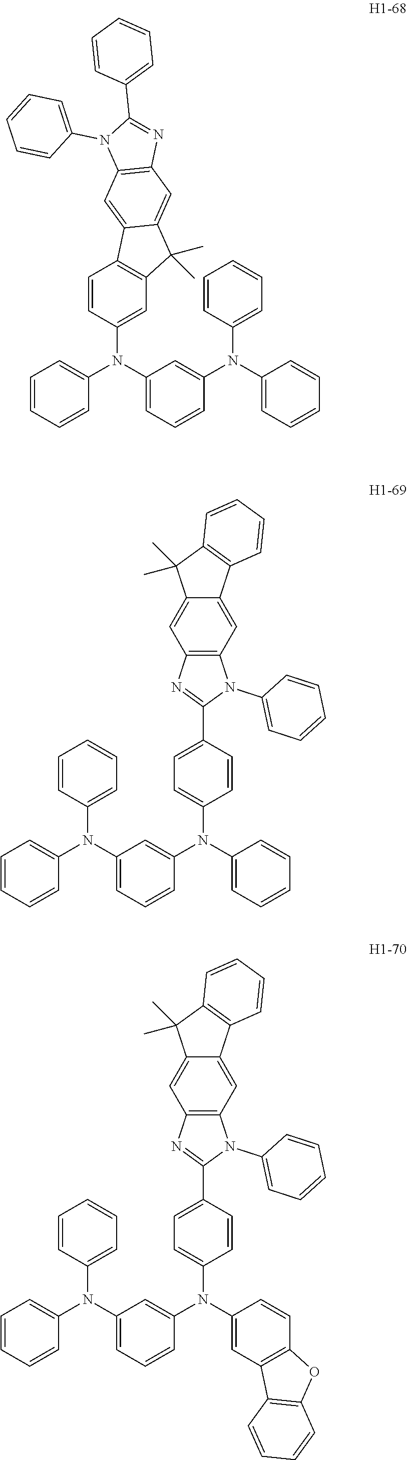 [SMILES: CC1(C)c2cc(N(c3ccccc3)c3cccc(N(c4ccccc4)c4ccccc4)c3)ccc2-c2cc3c(cc21)nc(-c1ccccc1)n3-c1ccccc1 ; CC1(C)c2ccccc2-c2cc3c(cc21)nc(-c1ccc(N(c2ccccc2)c2cccc(N(c4ccccc4)c4ccccc4)c2)cc1)n3-c1ccccc1 ; CC1(C)c2ccccc2-c2cc3c(cc21)nc(-c1ccc(N(c2cccc(N(c4ccccc4)c4ccccc4)c2)c2ccc4oc5ccccc5c4c2)cc1)n3-c1ccccc1]

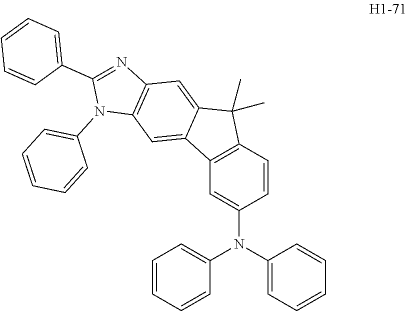 [SMILES: CC1(C)c2ccc(N(c3ccccc3)c3ccccc3)cc2-c2cc3c(cc21)nc(-c1ccccc1)n3-c1ccccc1]

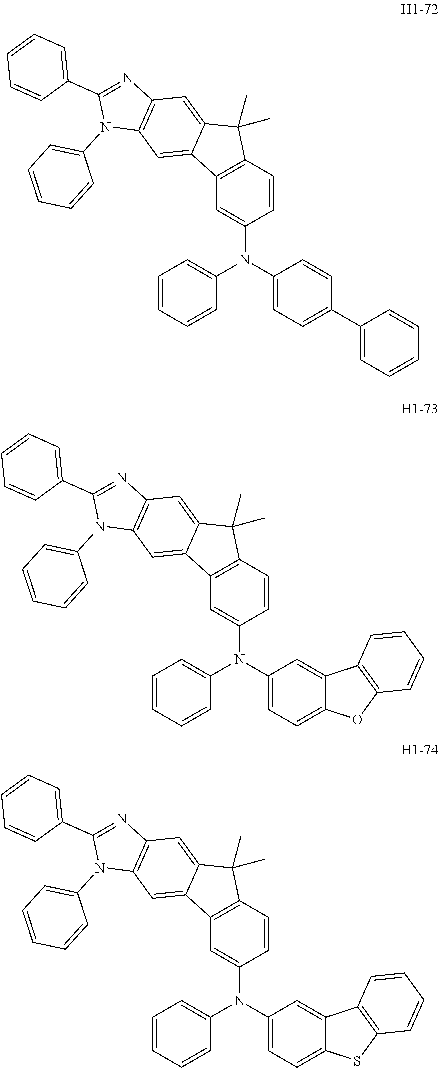 [SMILES: CC1(C)c2ccc(N(c3ccccc3)c3ccc(-c4ccccc4)cc3)cc2-c2cc3c(cc21)nc(-c1ccccc1)n3-c1ccccc1 ; CC1(C)c2ccc(N(c3ccccc3)c3ccc4oc5ccccc5c4c3)cc2-c2cc3c(cc21)nc(-c1ccccc1)n3-c1ccccc1 ; CC1(C)c2ccc(N(c3ccccc3)c3ccc4sc5ccccc5c4c3)cc2-c2cc3c(cc21)nc(-c1ccccc1)n3-c1ccccc1]

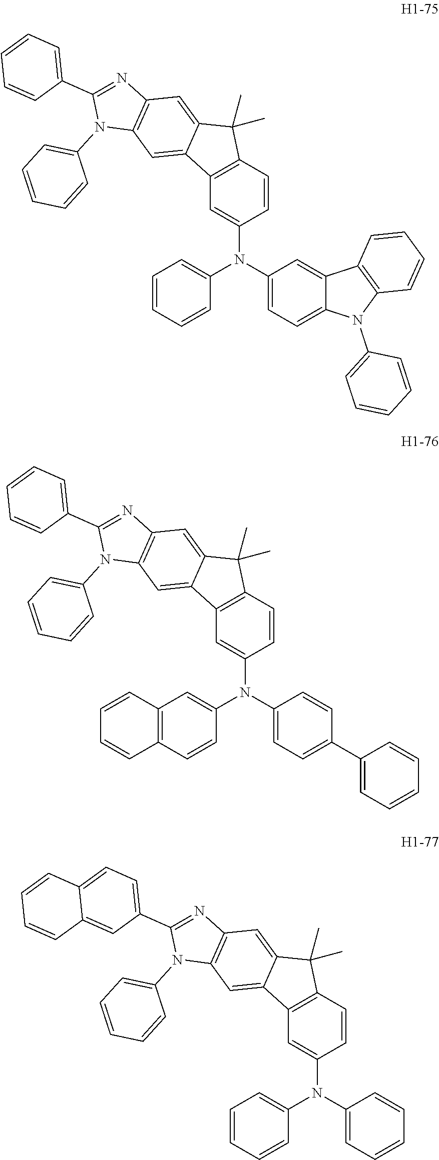 [SMILES: CC1(C)c2ccc(N(c3ccccc3)c3ccc4c(c3)c3ccccc3n4-c3ccccc3)cc2-c2cc3c(cc21)nc(-c1ccccc1)n3-c1ccccc1 ; CC1(C)c2ccc(N(c3ccc(-c4ccccc4)cc3)c3ccc4ccccc4c3)cc2-c2cc3c(cc21)nc(-c1ccccc1)n3-c1ccccc1 ; CC1(C)c2ccc(N(c3ccccc3)c3ccccc3)cc2-c2cc3c(cc21)nc(-c1ccc2ccccc2c1)n3-c1ccccc1]

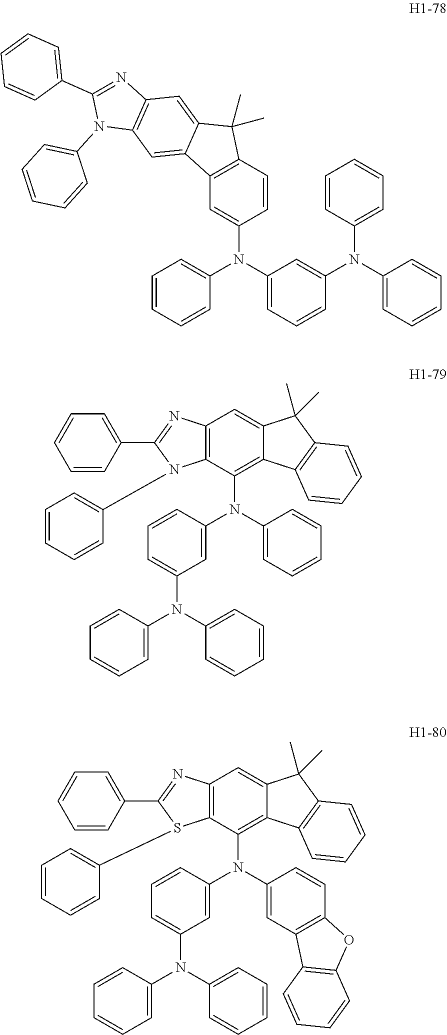

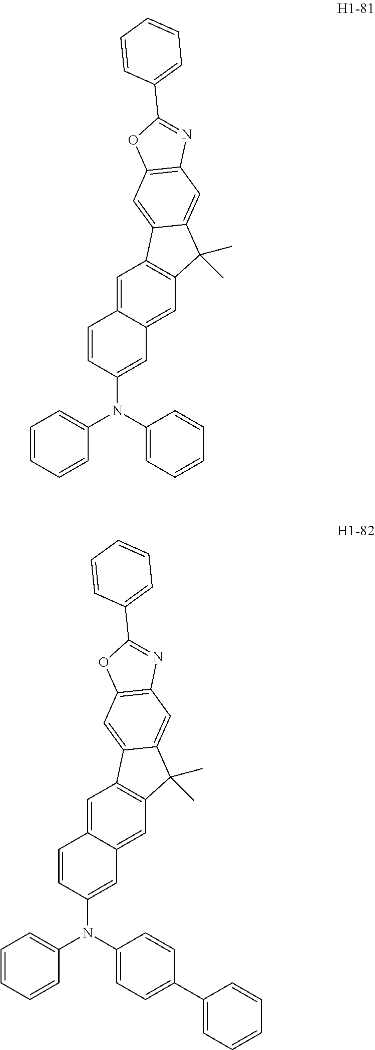 [SMILES: CC1(C)c2cc3cc(N(c4ccccc4)c4ccccc4)ccc3cc2-c2cc3oc(-c4ccccc4)nc3cc21 ; CC1(C)c2cc3cc(N(c4ccccc4)c4ccc(-c5ccccc5)cc4)ccc3cc2-c2cc3oc(-c4ccccc4)nc3cc21]

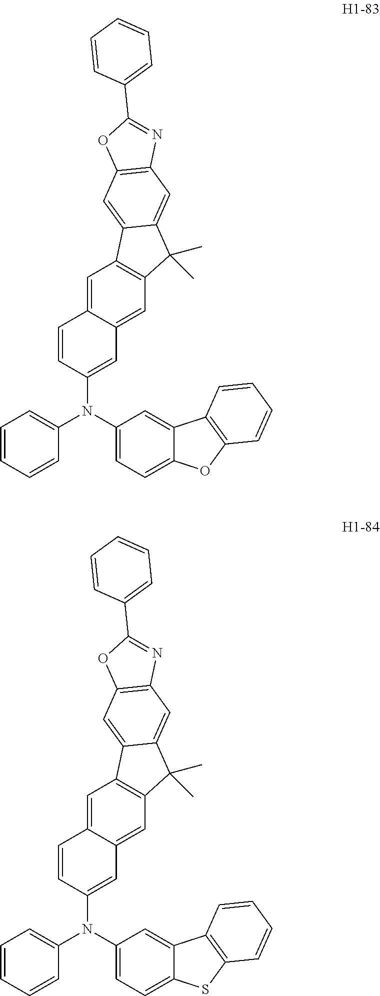 [SMILES: CC1(C)c2cc3cc(N(c4ccccc4)c4ccc5oc6ccccc6c5c4)ccc3cc2-c2cc3oc(-c4ccccc4)nc3cc21 ; CC1(C)c2cc3cc(N(c4ccccc4)c4ccc5sc6ccccc6c5c4)ccc3cc2-c2cc3oc(-c4ccccc4)nc3cc21]

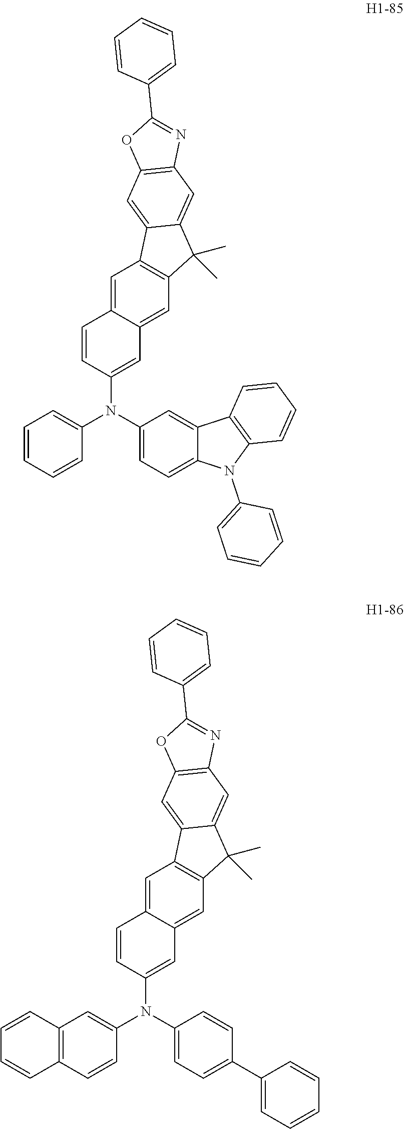 [SMILES: CC1(C)c2cc3cc(N(c4ccccc4)c4ccc5c(c4)c4ccccc4n5-c4ccccc4)ccc3cc2-c2cc3oc(-c4ccccc4)nc3cc21 ; CC1(C)c2cc3cc(N(c4ccc(-c5ccccc5)cc4)c4ccc5ccccc5c4)ccc3cc2-c2cc3oc(-c4ccccc4)nc3cc21]

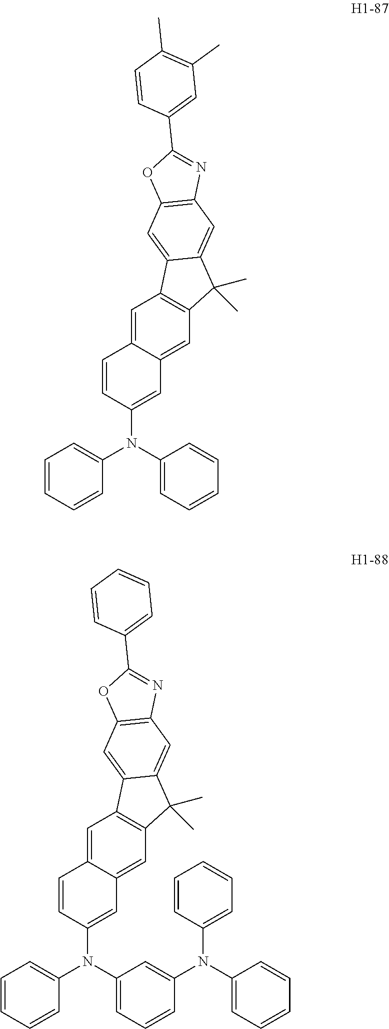 [SMILES: Cc1ccc(-c2nc3cc4c(cc3o2)-c2cc3ccc(N(c5ccccc5)c5ccccc5)cc3cc2C4(C)C)cc1C ; CC1(C)c2cc3cc(N(c4ccccc4)c4cccc(N(c5ccccc5)c5ccccc5)c4)ccc3cc2-c2cc3oc(-c4ccccc4)nc3cc21]

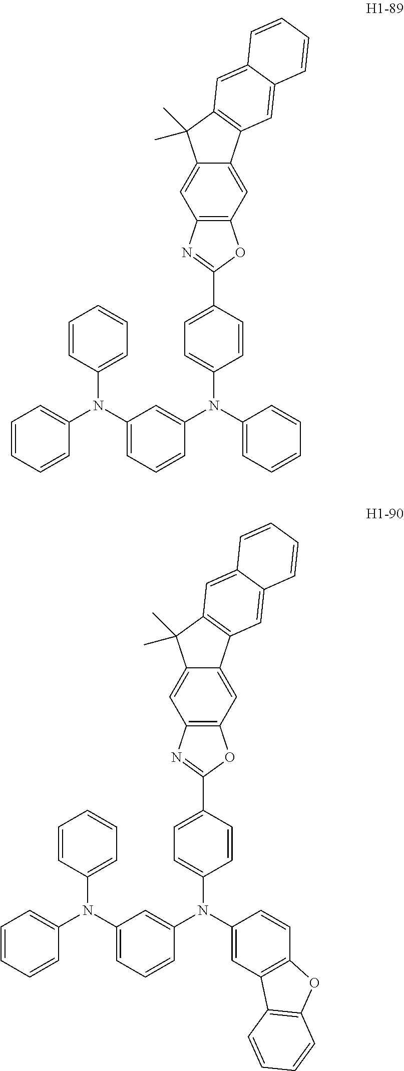 [SMILES: CC1(C)c2cc3ccccc3cc2-c2cc3oc(-c4ccc(N(c5ccccc5)c5cccc(N(c6ccccc6)c6ccccc6)c5)cc4)nc3cc21 ; CC1(C)c2cc3ccccc3cc2-c2cc3oc(-c4ccc(N(c5cccc(N(c6ccccc6)c6ccccc6)c5)c5ccc6oc7ccccc7c6c5)cc4)nc3cc21]

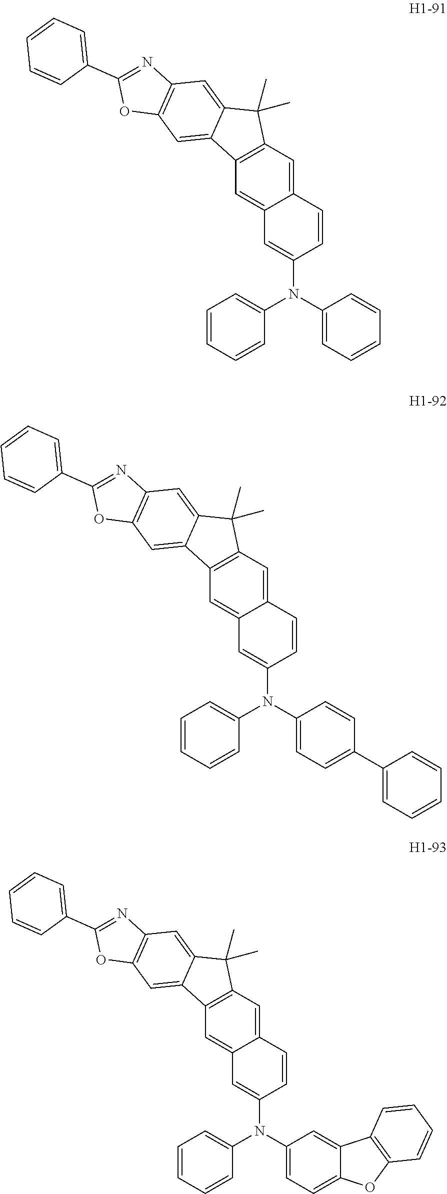 [SMILES: CC1(C)c2cc3ccc(N(c4ccccc4)c4ccccc4)cc3cc2-c2cc3oc(-c4ccccc4)nc3cc21 ; CC1(C)c2cc3ccc(N(c4ccccc4)c4ccc(-c5ccccc5)cc4)cc3cc2-c2cc3oc(-c4ccccc4)nc3cc21 ; CC1(C)c2cc3ccc(N(c4ccccc4)c4ccc5oc6ccccc6c5c4)cc3cc2-c2cc3oc(-c4ccccc4)nc3cc21]

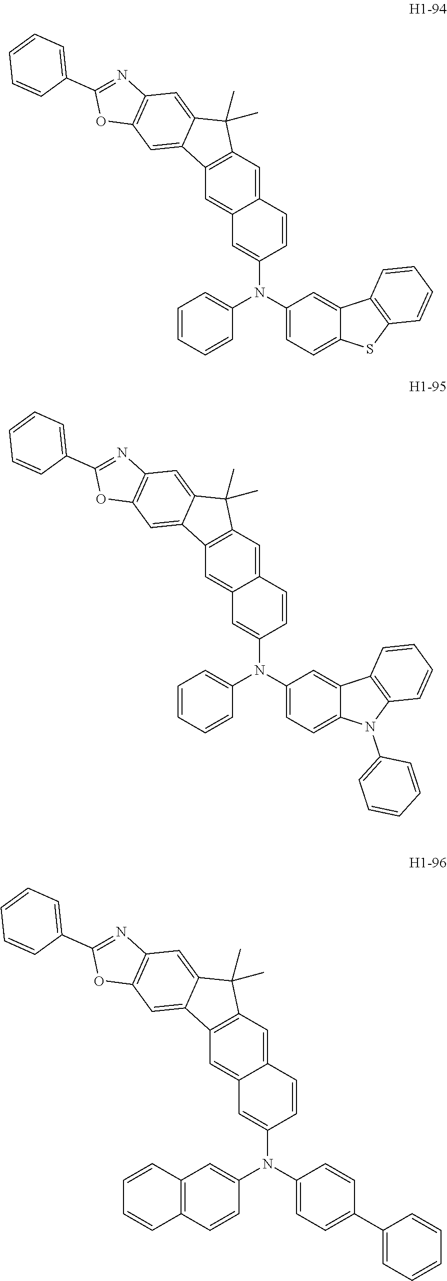 [SMILES: CC1(C)c2cc3ccc(N(c4ccccc4)c4ccc5sc6ccccc6c5c4)cc3cc2-c2cc3oc(-c4ccccc4)nc3cc21 ; CC1(C)c2cc3ccc(N(c4ccccc4)c4ccc5c(c4)c4ccccc4n5-c4ccccc4)cc3cc2-c2cc3oc(-c4ccccc4)nc3cc21 ; CC1(C)c2cc3ccc(N(c4ccc(-c5ccccc5)cc4)c4ccc5ccccc5c4)cc3cc2-c2cc3oc(-c4ccccc4)nc3cc21]

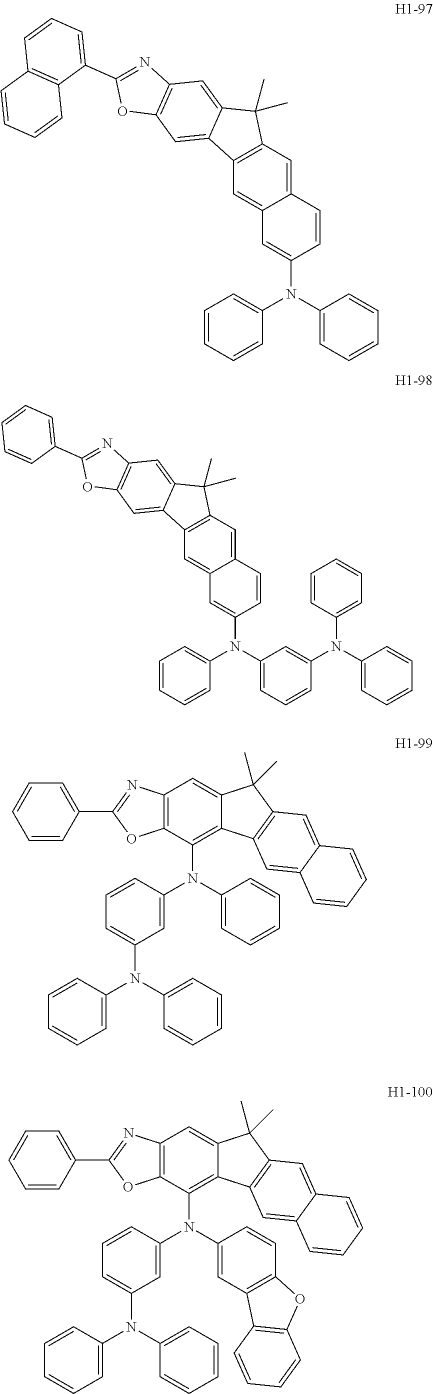 [SMILES: CC1(C)c2cc3ccc(N(c4ccccc4)c4ccccc4)cc3cc2-c2cc3oc(-c4cccc5ccccc45)nc3cc21 ; CC1(C)c2cc3ccc(N(c4ccccc4)c4cccc(N(c5ccccc5)c5ccccc5)c4)cc3cc2-c2cc3oc(-c4ccccc4)nc3cc21 ; CC1(C)c2cc3ccccc3cc2-c2c1cc1nc(-c3ccccc3)oc1c2N(c1ccccc1)c1cccc(N(c2ccccc2)c2ccccc2)c1 ; CC1(C)c2cc3ccccc3cc2-c2c1cc1nc(-c3ccccc3)oc1c2N(c1cccc(N(c2ccccc2)c2ccccc2)c1)c1ccc2oc3ccccc3c2c1]

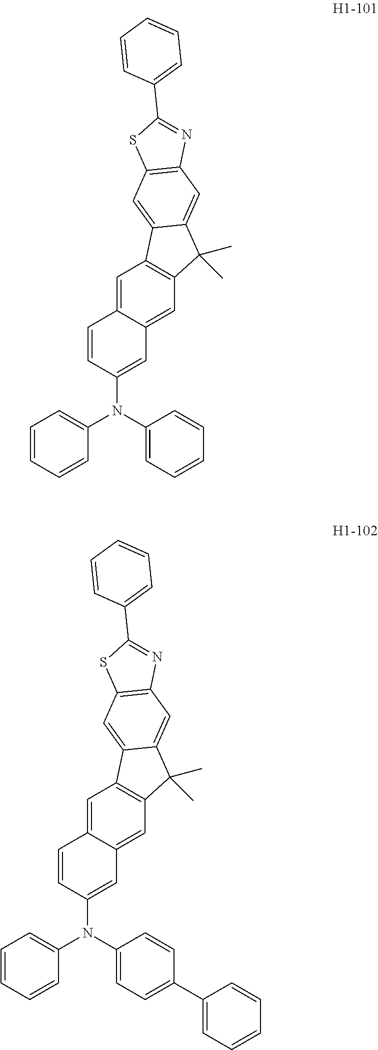 [SMILES: CC1(C)c2cc3cc(N(c4ccccc4)c4ccccc4)ccc3cc2-c2cc3sc(-c4ccccc4)nc3cc21 ; CC1(C)c2cc3cc(N(c4ccccc4)c4ccc(-c5ccccc5)cc4)ccc3cc2-c2cc3sc(-c4ccccc4)nc3cc21]

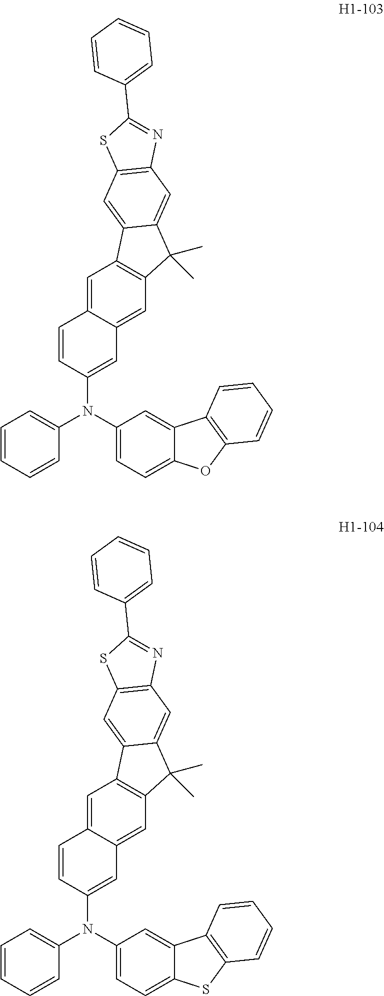 [SMILES: CC1(C)c2cc3cc(N(c4ccccc4)c4ccc5oc6ccccc6c5c4)ccc3cc2-c2cc3sc(-c4ccccc4)nc3cc21 ; CC1(C)c2cc3cc(N(c4ccccc4)c4ccc5sc6ccccc6c5c4)ccc3cc2-c2cc3sc(-c4ccccc4)nc3cc21]

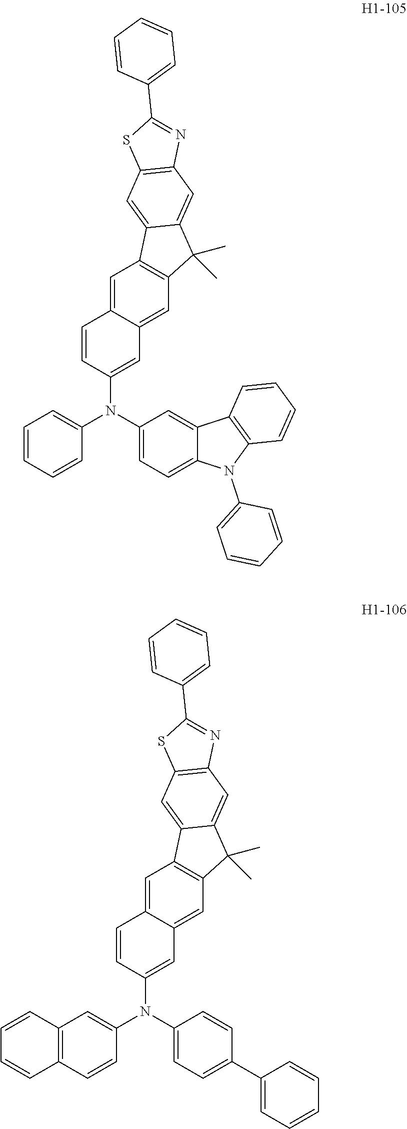 [SMILES: CC1(C)c2cc3cc(N(c4ccccc4)c4ccc5c(c4)c4ccccc4n5-c4ccccc4)ccc3cc2-c2cc3sc(-c4ccccc4)nc3cc21 ; CC1(C)c2cc3cc(N(c4ccc(-c5ccccc5)cc4)c4ccc5ccccc5c4)ccc3cc2-c2cc3sc(-c4ccccc4)nc3cc21]

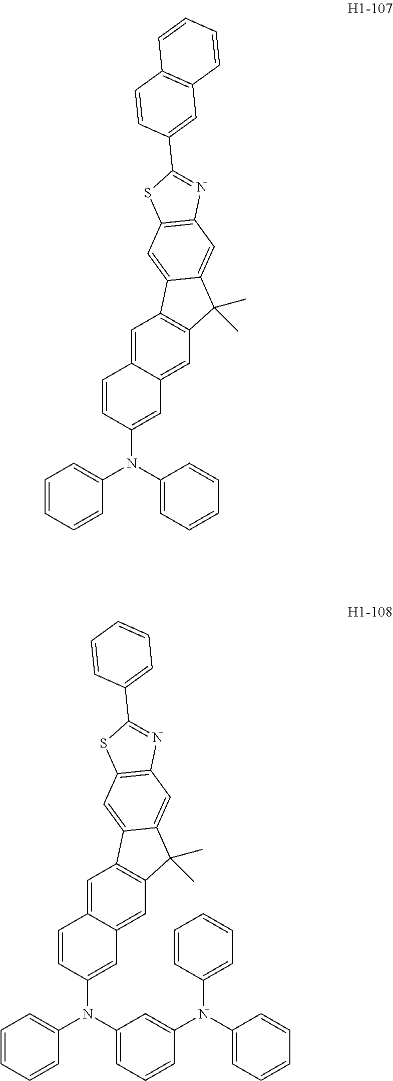 [SMILES: CC1(C)c2cc3cc(N(c4ccccc4)c4ccccc4)ccc3cc2-c2cc3sc(-c4ccc5ccccc5c4)nc3cc21 ; CC1(C)c2cc3cc(N(c4ccccc4)c4cccc(N(c5ccccc5)c5ccccc5)c4)ccc3cc2-c2cc3sc(-c4ccccc4)nc3cc21]

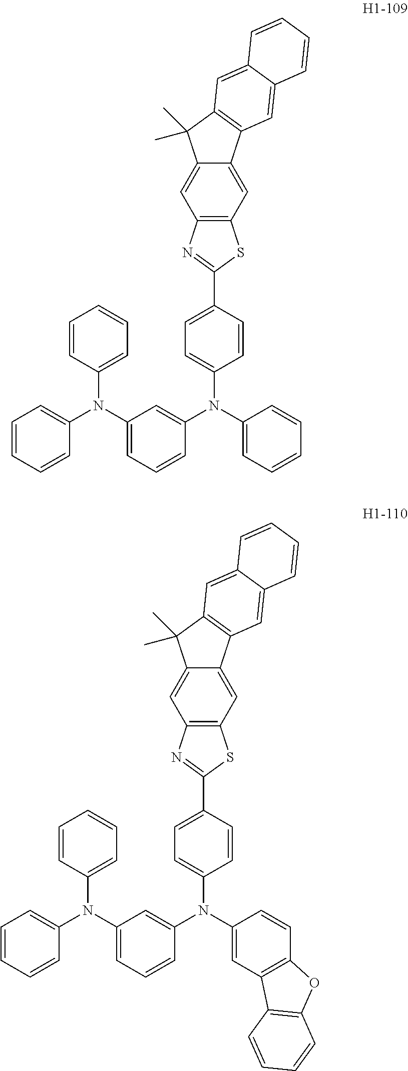 [SMILES: CC1(C)c2cc3ccccc3cc2-c2cc3sc(-c4ccc(N(c5ccccc5)c5cccc(N(c6ccccc6)c6ccccc6)c5)cc4)nc3cc21 ; CC1(C)c2cc3ccccc3cc2-c2cc3sc(-c4ccc(N(c5cccc(N(c6ccccc6)c6ccccc6)c5)c5ccc6oc7ccccc7c6c5)cc4)nc3cc21]

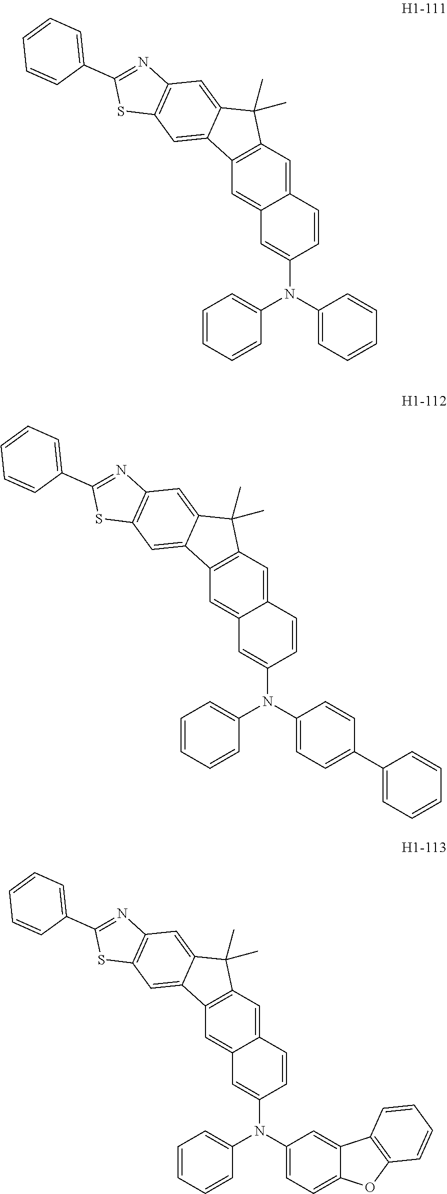 [SMILES: CC1(C)c2cc3ccc(N(c4ccccc4)c4ccccc4)cc3cc2-c2cc3sc(-c4ccccc4)nc3cc21 ; CC1(C)c2cc3ccc(N(c4ccccc4)c4ccc(-c5ccccc5)cc4)cc3cc2-c2cc3sc(-c4ccccc4)nc3cc21 ; CC1(C)c2cc3ccc(N(c4ccccc4)c4ccc5oc6ccccc6c5c4)cc3cc2-c2cc3sc(-c4ccccc4)nc3cc21]

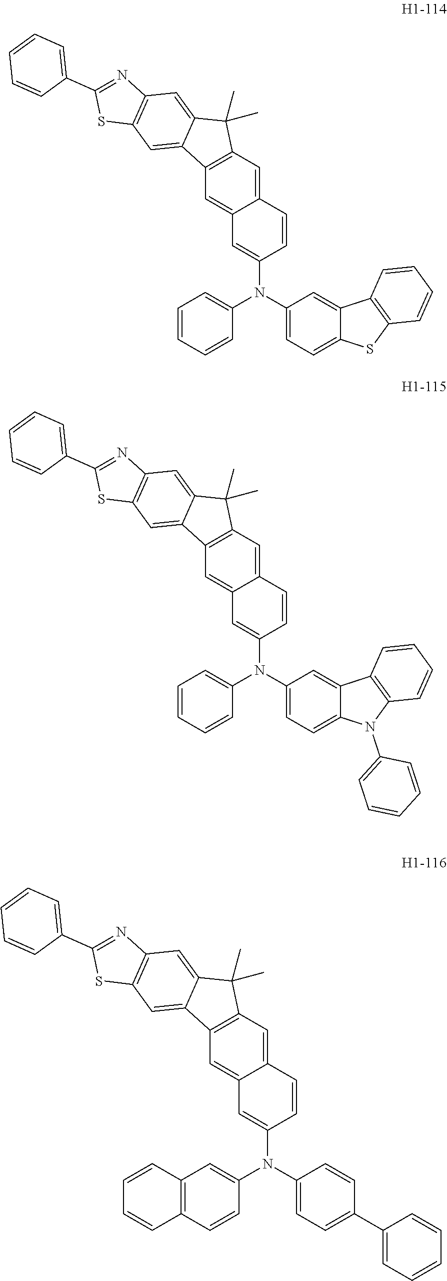 [SMILES: CC1(C)c2cc3ccc(N(c4ccccc4)c4ccc5sc6ccccc6c5c4)cc3cc2-c2cc3sc(-c4ccccc4)nc3cc21 ; CC1(C)c2cc3ccc(N(c4ccccc4)c4ccc5c(c4)c4ccccc4n5-c4ccccc4)cc3cc2-c2cc3sc(-c4ccccc4)nc3cc21 ; CC1(C)c2cc3ccc(N(c4ccc(-c5ccccc5)cc4)c4ccc5ccccc5c4)cc3cc2-c2cc3sc(-c4ccccc4)nc3cc21]

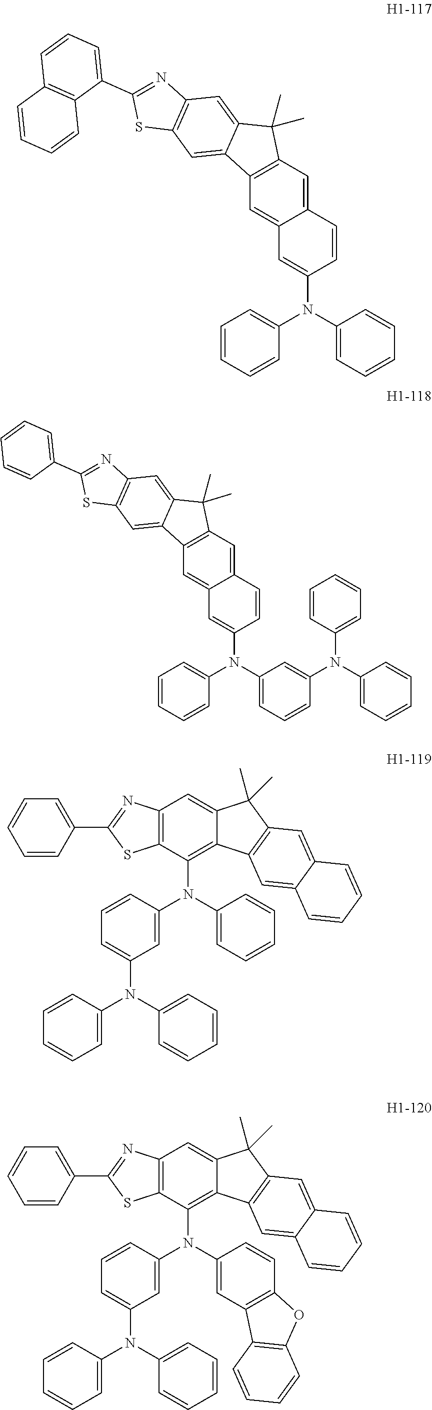 [SMILES: CC1(C)c2cc3ccc(N(c4ccccc4)c4ccccc4)cc3cc2-c2cc3sc(-c4cccc5ccccc45)nc3cc21 ; CC1(C)c2cc3ccc(N(c4ccccc4)c4cccc(N(c5ccccc5)c5ccccc5)c4)cc3cc2-c2cc3sc(-c4ccccc4)nc3cc21 ; CC1(C)c2cc3ccccc3cc2-c2c1cc1nc(-c3ccccc3)sc1c2N(c1ccccc1)c1cccc(N(c2ccccc2)c2ccccc2)c1 ; CC1(C)c2cc3ccccc3cc2-c2c1cc1nc(-c3ccccc3)sc1c2N(c1cccc(N(c2ccccc2)c2ccccc2)c1)c1ccc2oc3ccccc3c2c1]

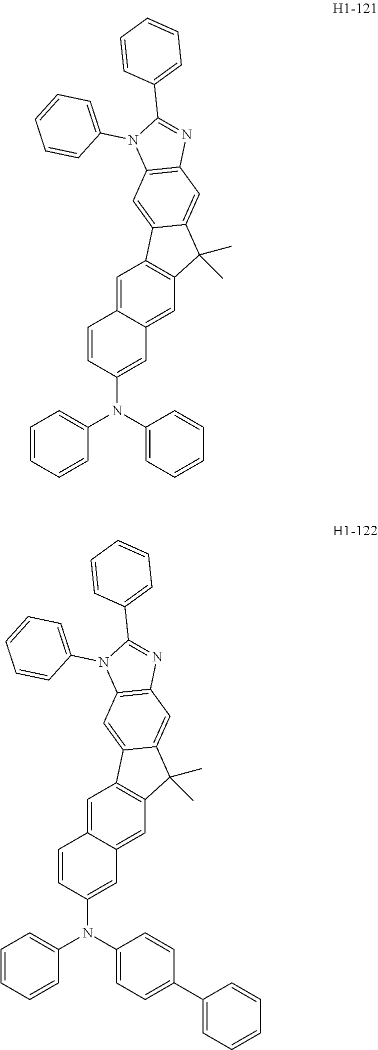 [SMILES: CC1(C)c2cc3cc(N(c4ccccc4)c4ccccc4)ccc3cc2-c2cc3c(cc21)nc(-c1ccccc1)n3-c1ccccc1 ; CC1(C)c2cc3cc(N(c4ccccc4)c4ccc(-c5ccccc5)cc4)ccc3cc2-c2cc3c(cc21)nc(-c1ccccc1)n3-c1ccccc1]

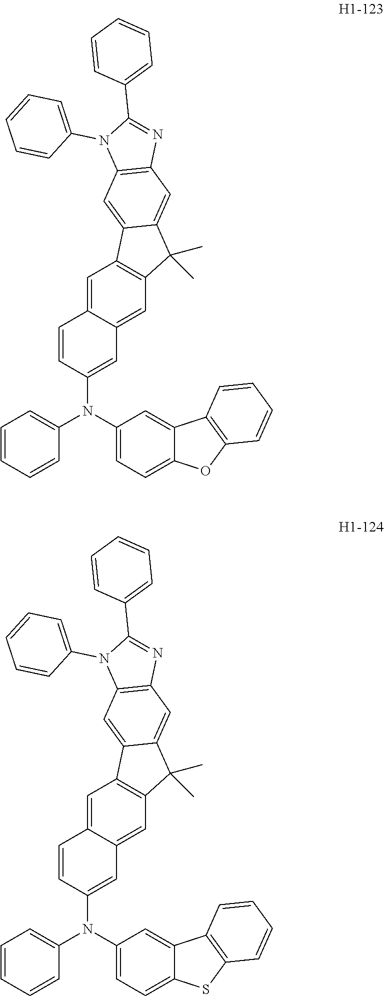 [SMILES: CC1(C)c2cc3cc(N(c4ccccc4)c4ccc5oc6ccccc6c5c4)ccc3cc2-c2cc3c(cc21)nc(-c1ccccc1)n3-c1ccccc1 ; CC1(C)c2cc3cc(N(c4ccccc4)c4ccc5sc6ccccc6c5c4)ccc3cc2-c2cc3c(cc21)nc(-c1ccccc1)n3-c1ccccc1]

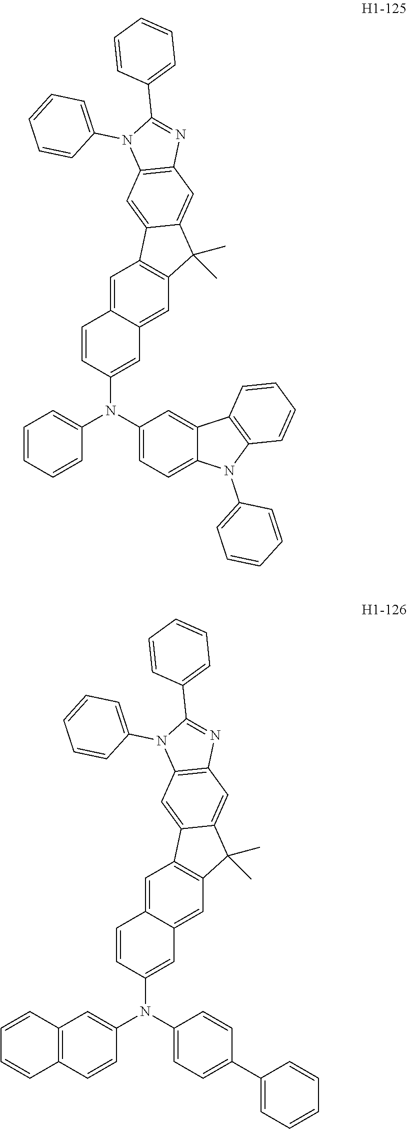 [SMILES: CC1(C)c2cc3cc(N(c4ccccc4)c4ccc5c(c4)c4ccccc4n5-c4ccccc4)ccc3cc2-c2cc3c(cc21)nc(-c1ccccc1)n3-c1ccccc1 ; CC1(C)c2cc3cc(N(c4ccc(-c5ccccc5)cc4)c4ccc5ccccc5c4)ccc3cc2-c2cc3c(cc21)nc(-c1ccccc1)n3-c1ccccc1]

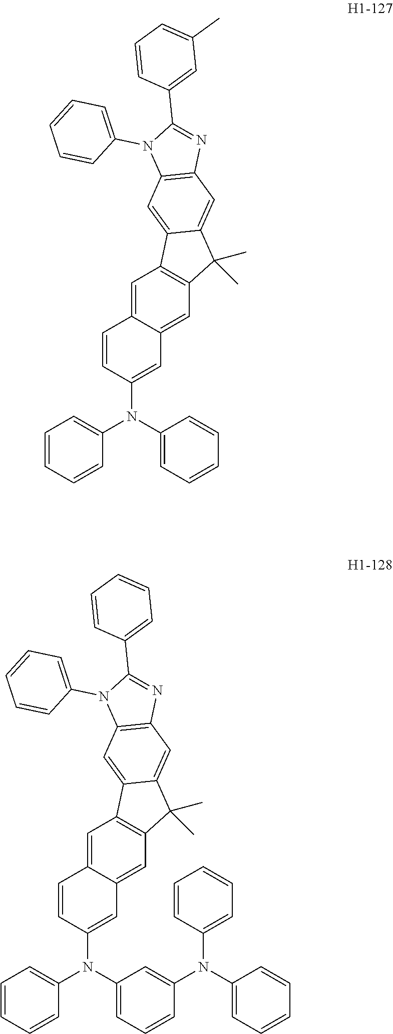 [SMILES: Cc1cccc(-c2nc3cc4c(cc3n2-c2ccccc2)-c2cc3ccc(N(c5ccccc5)c5ccccc5)cc3cc2C4(C)C)c1 ; CC1(C)c2cc3cc(N(c4ccccc4)c4cccc(N(c5ccccc5)c5ccccc5)c4)ccc3cc2-c2cc3c(cc21)nc(-c1ccccc1)n3-c1ccccc1]

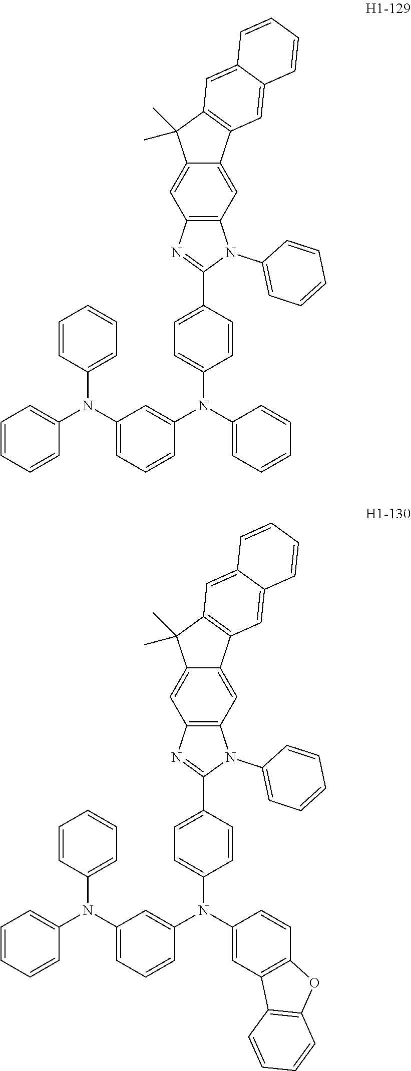 [SMILES: CC1(C)c2cc3ccccc3cc2-c2cc3c(cc21)nc(-c1ccc(N(c2ccccc2)c2cccc(N(c4ccccc4)c4ccccc4)c2)cc1)n3-c1ccccc1 ; CC1(C)c2cc3ccccc3cc2-c2cc3c(cc21)nc(-c1ccc(N(c2cccc(N(c4ccccc4)c4ccccc4)c2)c2ccc4oc5ccccc5c4c2)cc1)n3-c1ccccc1]

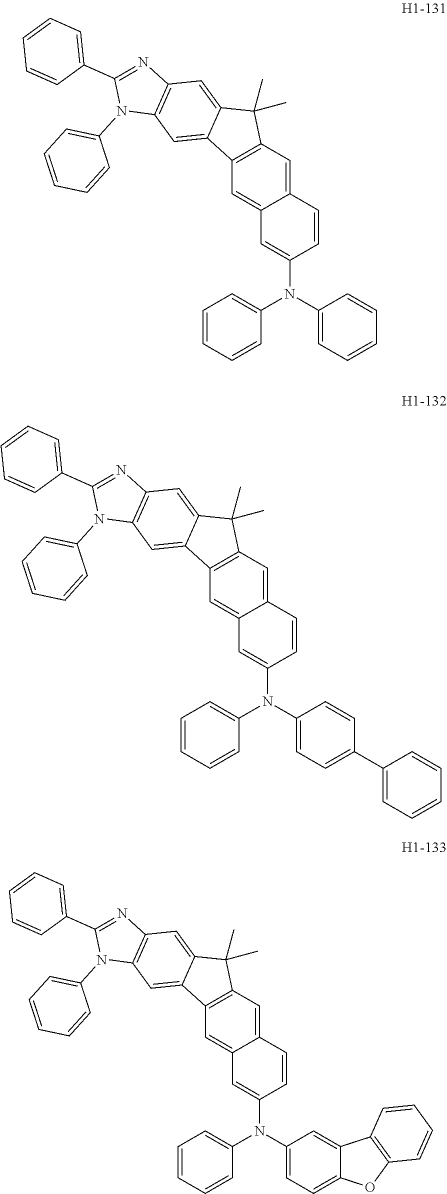 [SMILES: CC1(C)c2cc3ccc(N(c4ccccc4)c4ccccc4)cc3cc2-c2cc3c(cc21)nc(-c1ccccc1)n3-c1ccccc1 ; CC1(C)c2cc3ccc(N(c4ccccc4)c4ccc(-c5ccccc5)cc4)cc3cc2-c2cc3c(cc21)nc(-c1ccccc1)n3-c1ccccc1 ; CC1(C)c2cc3ccc(N(c4ccccc4)c4ccc5oc6ccccc6c5c4)cc3cc2-c2cc3c(cc21)nc(-c1ccccc1)n3-c1ccccc1]

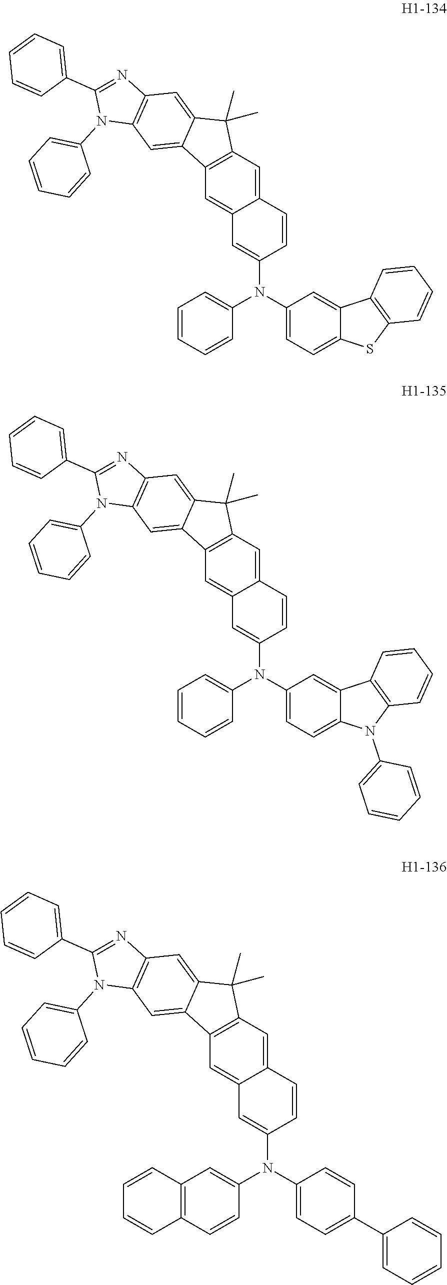 [SMILES: CC1(C)c2cc3ccc(N(c4ccccc4)c4ccc5sc6ccccc6c5c4)cc3cc2-c2cc3c(cc21)nc(-c1ccccc1)n3-c1ccccc1 ; CC1(C)c2cc3ccc(N(c4ccccc4)c4ccc5c(c4)c4ccccc4n5-c4ccccc4)cc3cc2-c2cc3c(cc21)nc(-c1ccccc1)n3-c1ccccc1 ; CC1(C)c2cc3ccc(N(c4ccc(-c5ccccc5)cc4)c4ccc5ccccc5c4)cc3cc2-c2cc3c(cc21)nc(-c1ccccc1)n3-c1ccccc1]

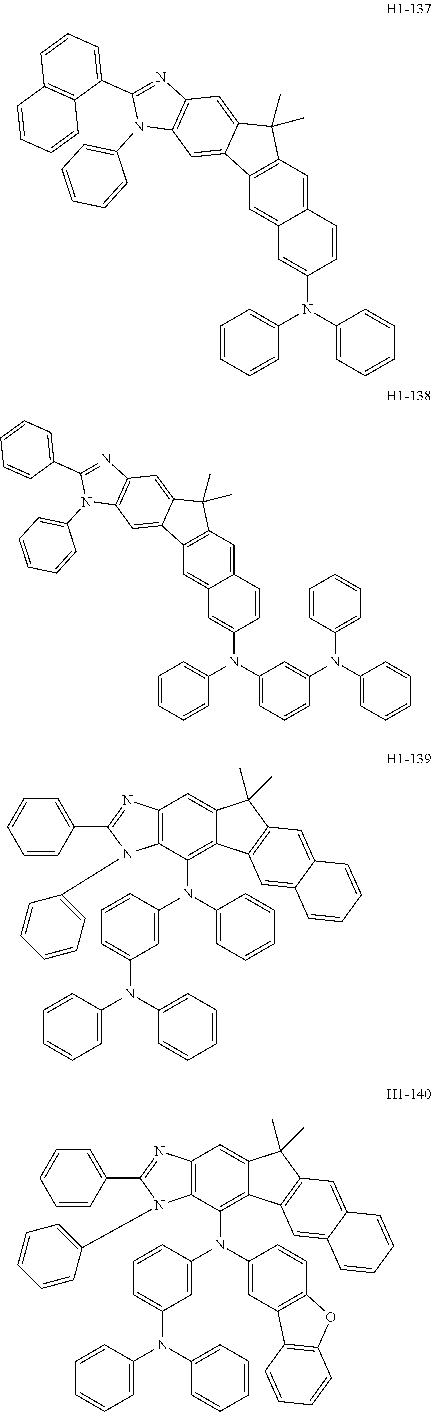 [SMILES: CC1(C)c2cc3ccc(N(c4ccccc4)c4ccccc4)cc3cc2-c2cc3c(cc21)nc(-c1cccc2ccccc12)n3-c1ccccc1 ; CC1(C)c2cc3ccc(N(c4ccccc4)c4cccc(N(c5ccccc5)c5ccccc5)c4)cc3cc2-c2cc3c(cc21)nc(-c1ccccc1)n3-c1ccccc1 ; CC1(C)c2cc3ccccc3cc2-c2c1cc1nc(-c3ccccc3)n(-c3ccccc3)c1c2N(c1ccccc1)c1cccc(N(c2ccccc2)c2ccccc2)c1 ; CC1(C)c2cc3ccccc3cc2-c2c1cc1nc(-c3ccccc3)n(-c3ccccc3)c1c2N(c1cccc(N(c2ccccc2)c2ccccc2)c1)c1ccc2oc3ccccc3c2c1]

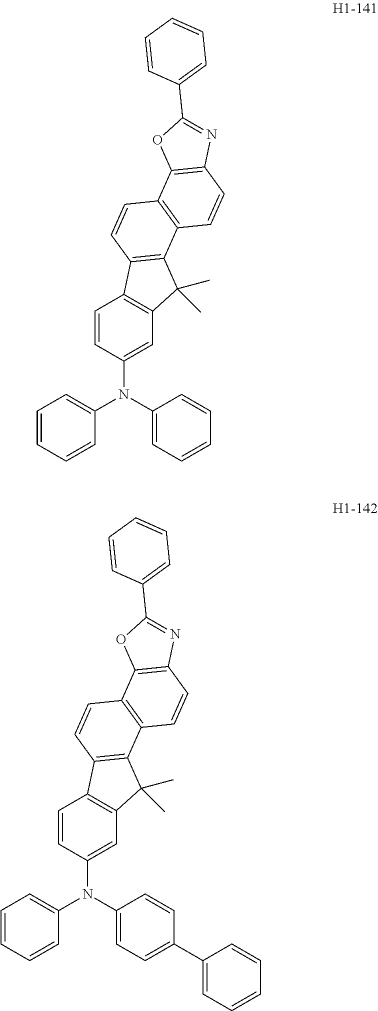 [SMILES: CC1(C)c2cc(N(c3ccccc3)c3ccccc3)ccc2-c2ccc3c(ccc4nc(-c5ccccc5)oc43)c21 ; CC1(C)c2cc(N(c3ccccc3)c3ccc(-c4ccccc4)cc3)ccc2-c2ccc3c(ccc4nc(-c5ccccc5)oc43)c21]

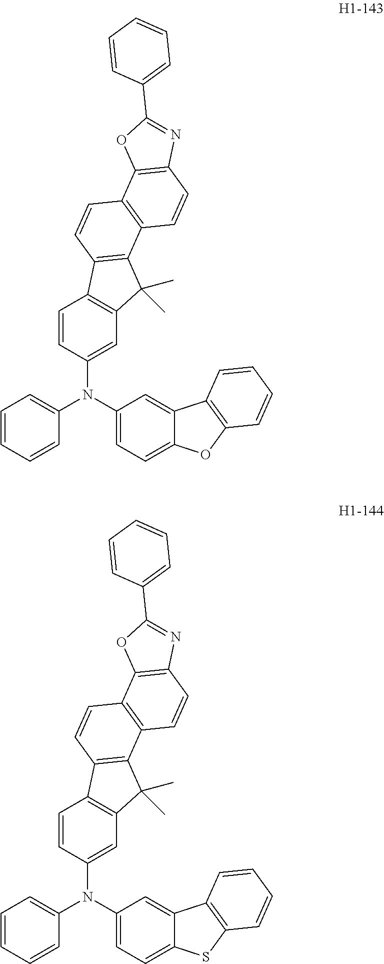 [SMILES: CC1(C)c2cc(N(c3ccccc3)c3ccc4oc5ccccc5c4c3)ccc2-c2ccc3c(ccc4nc(-c5ccccc5)oc43)c21 ; CC1(C)c2cc(N(c3ccccc3)c3ccc4sc5ccccc5c4c3)ccc2-c2ccc3c(ccc4nc(-c5ccccc5)oc43)c21]

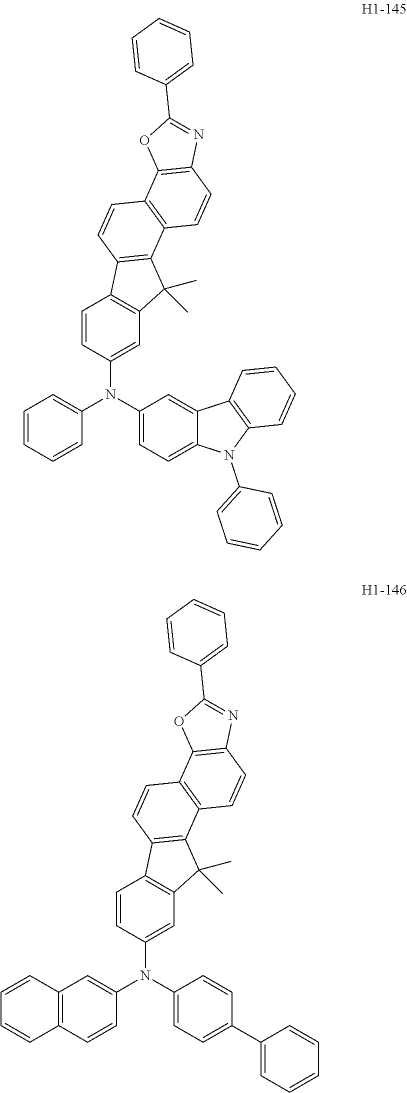 [SMILES: CC1(C)c2cc(N(c3ccccc3)c3ccc4c(c3)c3ccccc3n4-c3ccccc3)ccc2-c2ccc3c(ccc4nc(-c5ccccc5)oc43)c21 ; CC1(C)c2cc(N(c3ccc(-c4ccccc4)cc3)c3ccc4ccccc4c3)ccc2-c2ccc3c(ccc4nc(-c5ccccc5)oc43)c21]

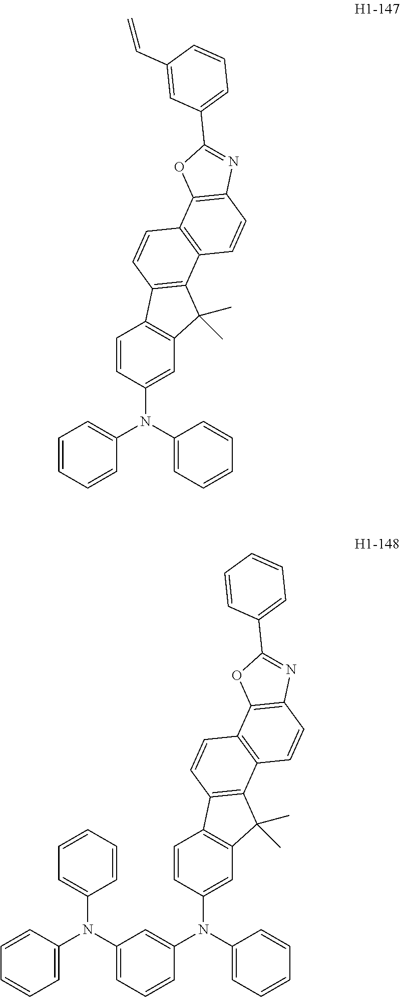 [SMILES: C=Cc1cccc(-c2nc3ccc4c5c(ccc4c3o2)-c2ccc(N(c3ccccc3)c3ccccc3)cc2C5(C)C)c1 ; CC1(C)c2cc(N(c3ccccc3)c3cccc(N(c4ccccc4)c4ccccc4)c3)ccc2-c2ccc3c(ccc4nc(-c5ccccc5)oc43)c21]

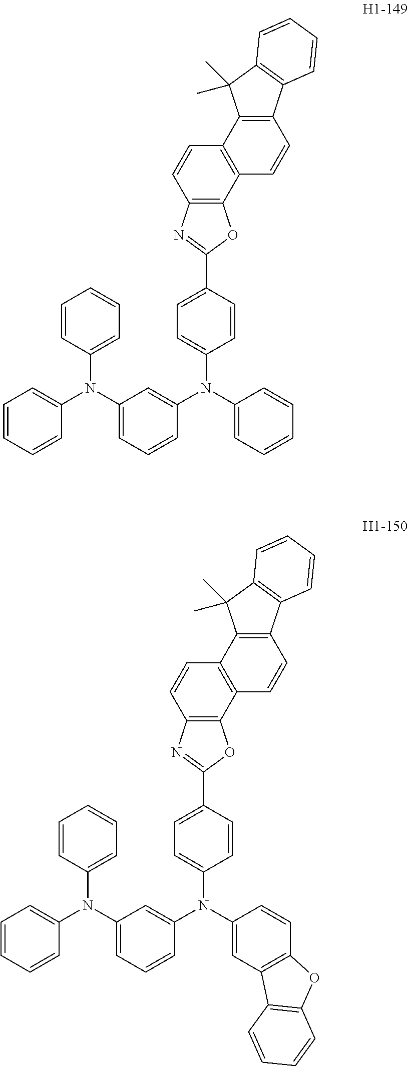 [SMILES: CC1(C)c2ccccc2-c2ccc3c(ccc4nc(-c5ccc(N(c6ccccc6)c6cccc(N(c7ccccc7)c7ccccc7)c6)cc5)oc43)c21 ; CC1(C)c2ccccc2-c2ccc3c(ccc4nc(-c5ccc(N(c6cccc(N(c7ccccc7)c7ccccc7)c6)c6ccc7oc8ccccc8c7c6)cc5)oc43)c21]

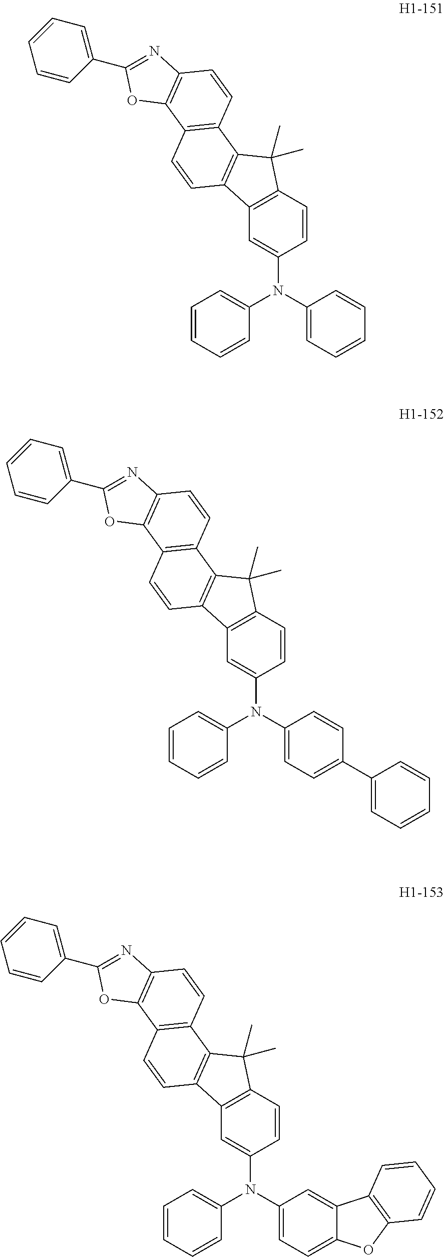 [SMILES: CC1(C)c2ccc(N(c3ccccc3)c3ccccc3)cc2-c2ccc3c(ccc4nc(-c5ccccc5)oc43)c21 ; CC1(C)c2ccc(N(c3ccccc3)c3ccc(-c4ccccc4)cc3)cc2-c2ccc3c(ccc4nc(-c5ccccc5)oc43)c21 ; CC1(C)c2ccc(N(c3ccccc3)c3ccc4oc5ccccc5c4c3)cc2-c2ccc3c(ccc4nc(-c5ccccc5)oc43)c21]

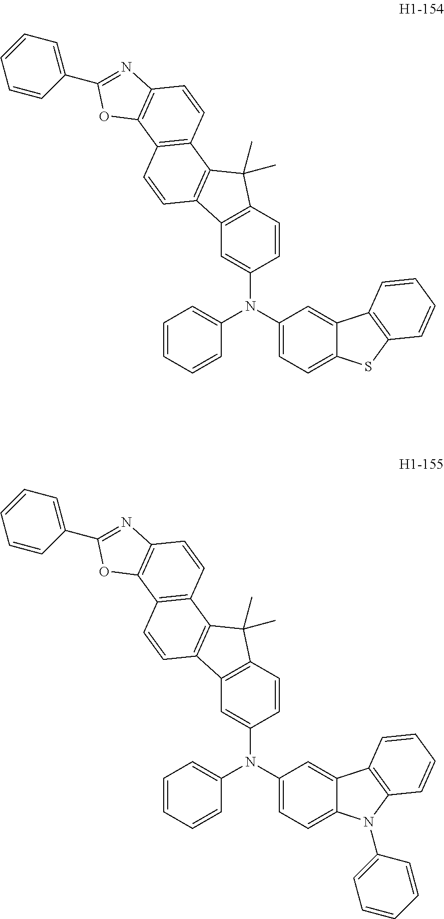 [SMILES: CC1(C)c2ccc(N(c3ccccc3)c3ccc4sc5ccccc5c4c3)cc2-c2ccc3c(ccc4nc(-c5ccccc5)oc43)c21 ; CC1(C)c2ccc(N(c3ccccc3)c3ccc4c(c3)c3ccccc3n4-c3ccccc3)cc2-c2ccc3c(ccc4nc(-c5ccccc5)oc43)c21]

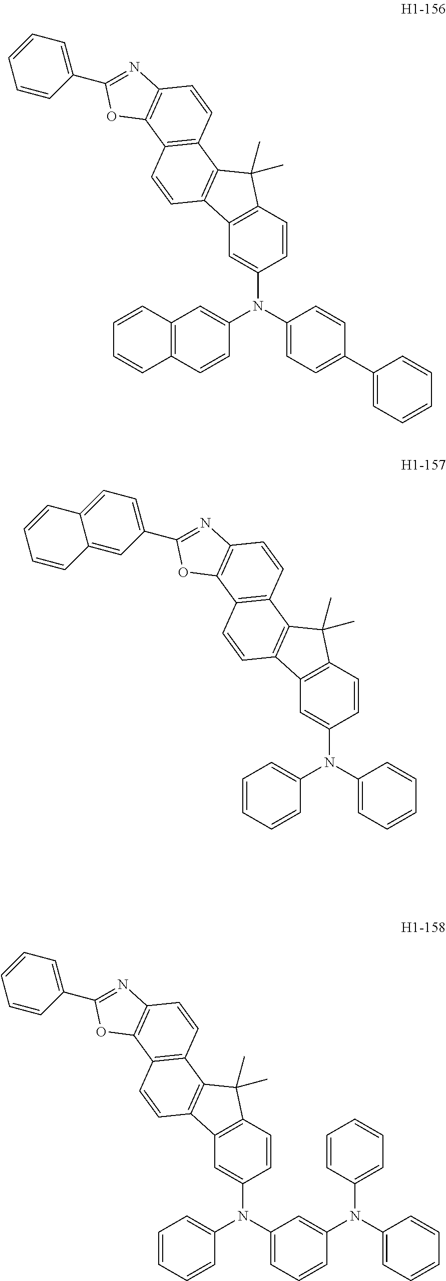 [SMILES: CC1(C)c2ccc(N(c3ccc(-c4ccccc4)cc3)c3ccc4ccccc4c3)cc2-c2ccc3c(ccc4nc(-c5ccccc5)oc43)c21 ; CC1(C)c2ccc(N(c3ccccc3)c3ccccc3)cc2-c2ccc3c(ccc4nc(-c5ccc6ccccc6c5)oc43)c21 ; CC1(C)c2ccc(N(c3ccccc3)c3cccc(N(c4ccccc4)c4ccccc4)c3)cc2-c2ccc3c(ccc4nc(-c5ccccc5)oc43)c21]

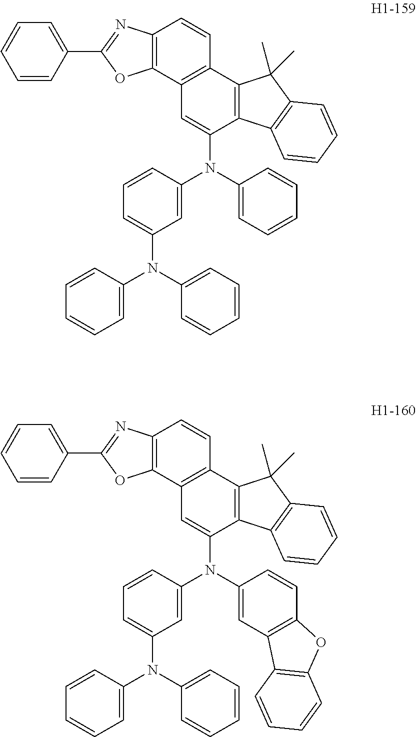 [SMILES: CC1(C)c2ccccc2-c2c(N(c3ccccc3)c3cccc(N(c4ccccc4)c4ccccc4)c3)cc3c(ccc4nc(-c5ccccc5)oc43)c21 ; CC1(C)c2ccccc2-c2c(N(c3cccc(N(c4ccccc4)c4ccccc4)c3)c3ccc4oc5ccccc5c4c3)cc3c(ccc4nc(-c5ccccc5)oc43)c21]

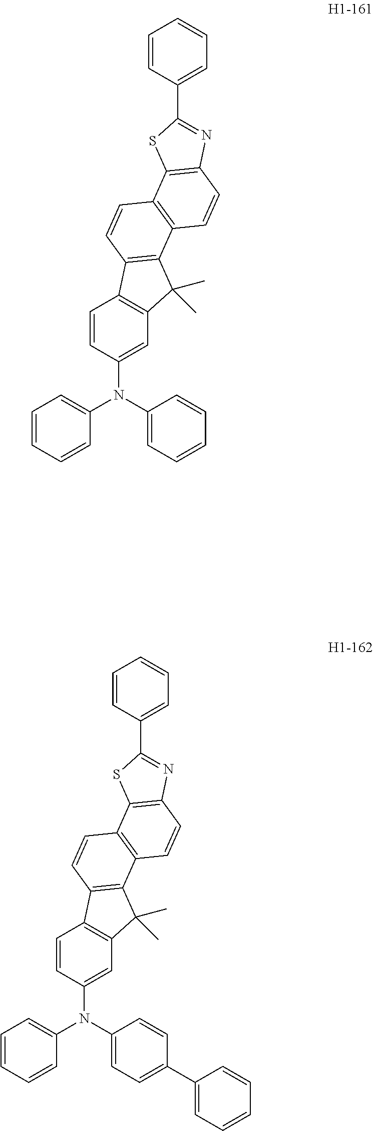 [SMILES: CC1(C)c2cc(N(c3ccccc3)c3ccccc3)ccc2-c2ccc3c(ccc4nc(-c5ccccc5)sc43)c21 ; CC1(C)c2cc(N(c3ccccc3)c3ccc(-c4ccccc4)cc3)ccc2-c2ccc3c(ccc4nc(-c5ccccc5)sc43)c21]

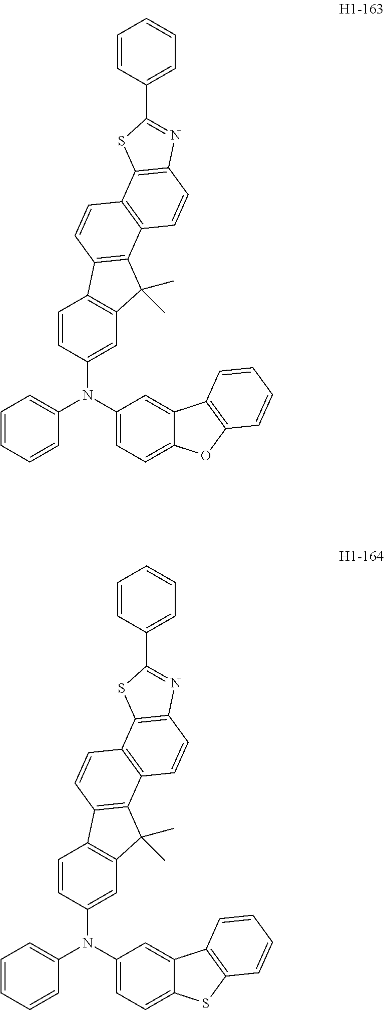 [SMILES: CC1(C)c2cc(N(c3ccccc3)c3ccc4oc5ccccc5c4c3)ccc2-c2ccc3c(ccc4nc(-c5ccccc5)sc43)c21 ; CC1(C)c2cc(N(c3ccccc3)c3ccc4sc5ccccc5c4c3)ccc2-c2ccc3c(ccc4nc(-c5ccccc5)sc43)c21]

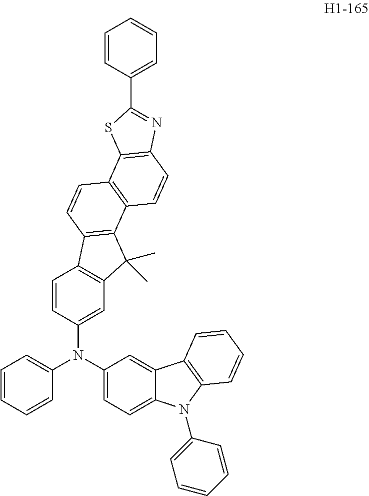 [SMILES: CC1(C)c2cc(N(c3ccccc3)c3ccc4c(c3)c3ccccc3n4-c3ccccc3)ccc2-c2ccc3c(ccc4nc(-c5ccccc5)sc43)c21]

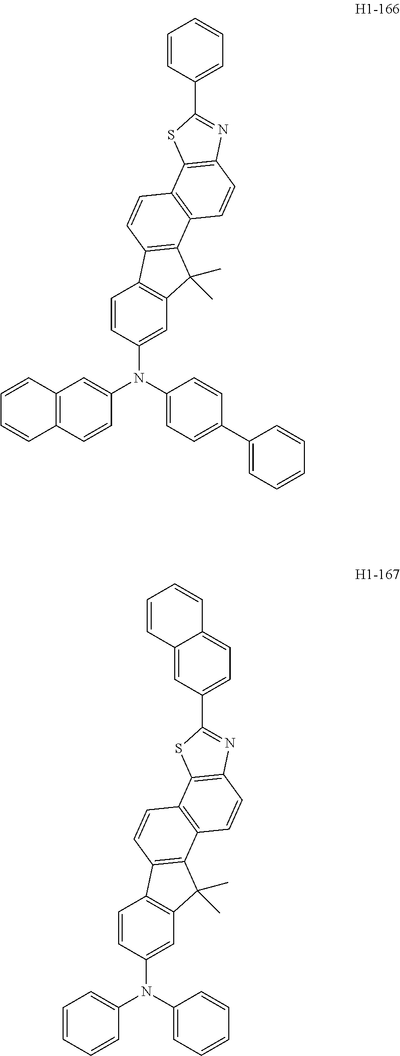 [SMILES: CC1(C)c2cc(N(c3ccc(-c4ccccc4)cc3)c3ccc4ccccc4c3)ccc2-c2ccc3c(ccc4nc(-c5ccccc5)sc43)c21 ; CC1(C)c2cc(N(c3ccccc3)c3ccccc3)ccc2-c2ccc3c(ccc4nc(-c5ccc6ccccc6c5)sc43)c21]

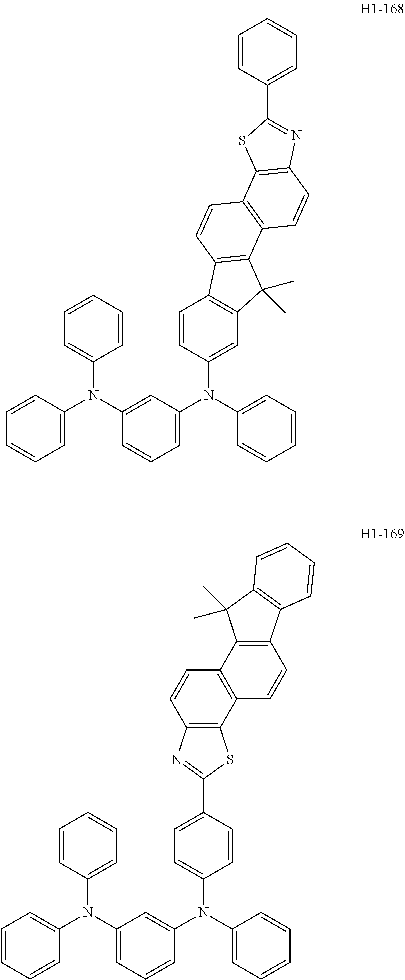 [SMILES: CC1(C)c2cc(N(c3ccccc3)c3cccc(N(c4ccccc4)c4ccccc4)c3)ccc2-c2ccc3c(ccc4nc(-c5ccccc5)sc43)c21 ; CC1(C)c2ccccc2-c2ccc3c(ccc4nc(-c5ccc(N(c6ccccc6)c6cccc(N(c7ccccc7)c7ccccc7)c6)cc5)sc43)c21]

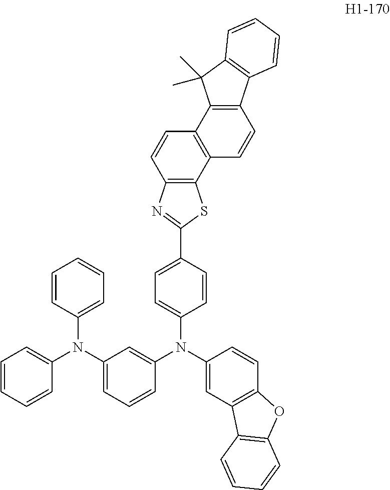 [SMILES: CC1(C)c2ccccc2-c2ccc3c(ccc4nc(-c5ccc(N(c6cccc(N(c7ccccc7)c7ccccc7)c6)c6ccc7oc8ccccc8c7c6)cc5)sc43)c21]

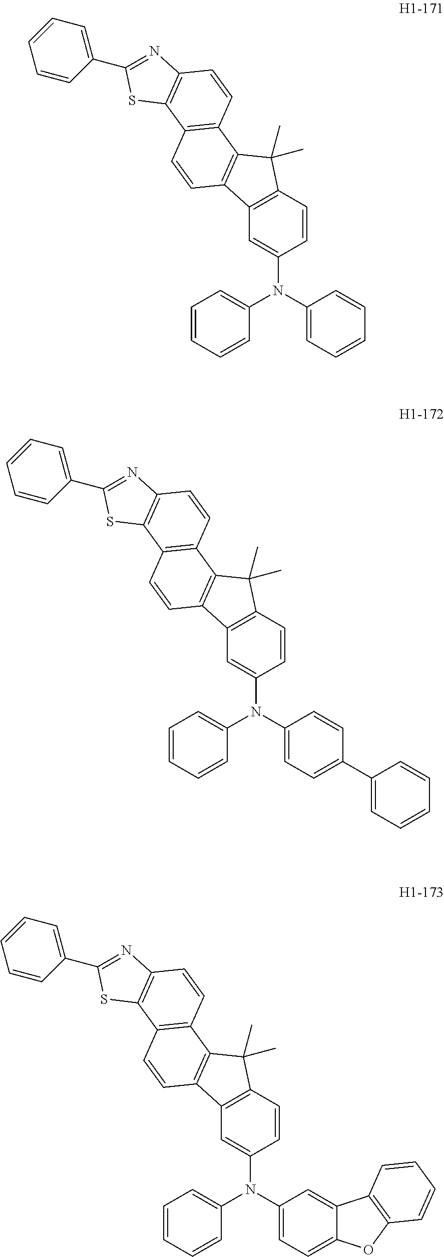 [SMILES: CC1(C)c2ccc(N(c3ccccc3)c3ccccc3)cc2-c2ccc3c(ccc4nc(-c5ccccc5)sc43)c21 ; CC1(C)c2ccc(N(c3ccccc3)c3ccc(-c4ccccc4)cc3)cc2-c2ccc3c(ccc4nc(-c5ccccc5)sc43)c21 ; CC1(C)c2ccc(N(c3ccccc3)c3ccc4oc5ccccc5c4c3)cc2-c2ccc3c(ccc4nc(-c5ccccc5)sc43)c21]

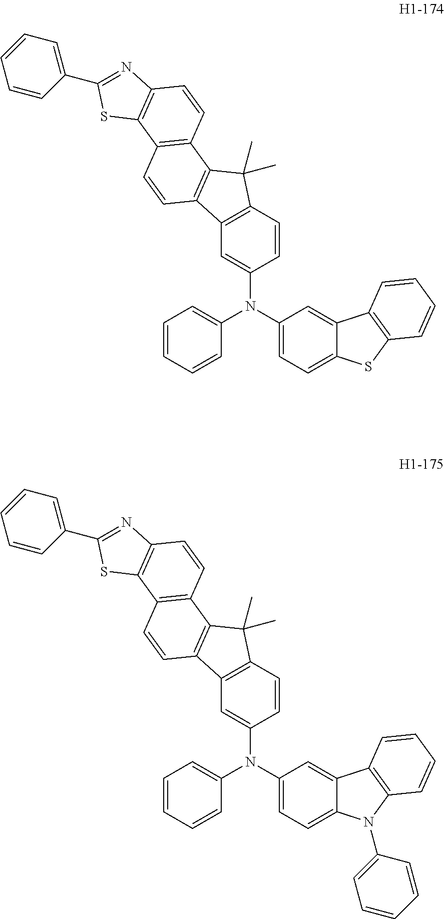 [SMILES: CC1(C)c2ccc(N(c3ccccc3)c3ccc4sc5ccccc5c4c3)cc2-c2ccc3c(ccc4nc(-c5ccccc5)sc43)c21 ; CC1(C)c2ccc(N(c3ccccc3)c3ccc4c(c3)c3ccccc3n4-c3ccccc3)cc2-c2ccc3c(ccc4nc(-c5ccccc5)sc43)c21]

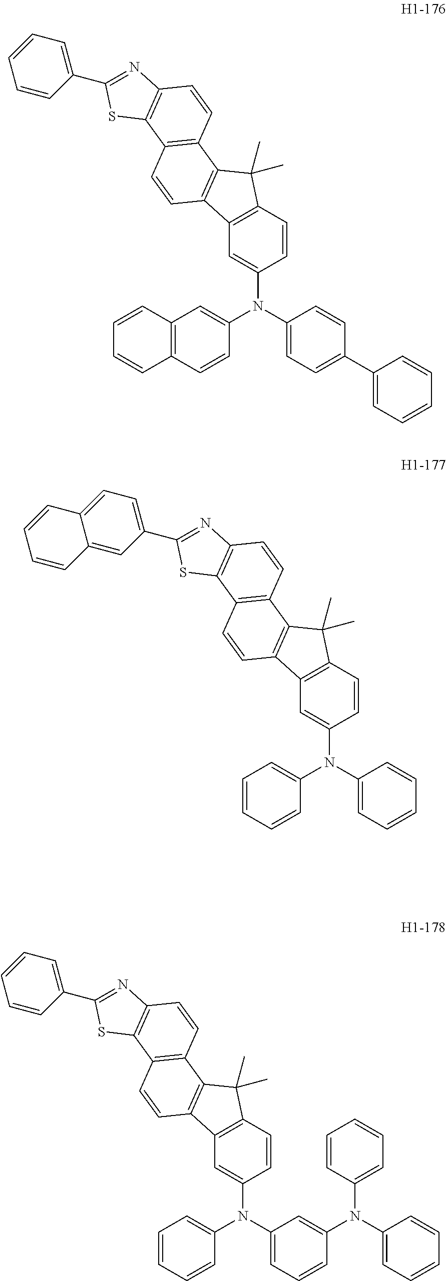 [SMILES: CC1(C)c2ccc(N(c3ccc(-c4ccccc4)cc3)c3ccc4ccccc4c3)cc2-c2ccc3c(ccc4nc(-c5ccccc5)sc43)c21 ; CC1(C)c2ccc(N(c3ccccc3)c3ccccc3)cc2-c2ccc3c(ccc4nc(-c5ccc6ccccc6c5)sc43)c21 ; CC1(C)c2ccc(N(c3ccccc3)c3cccc(N(c4ccccc4)c4ccccc4)c3)cc2-c2ccc3c(ccc4nc(-c5ccccc5)sc43)c21]

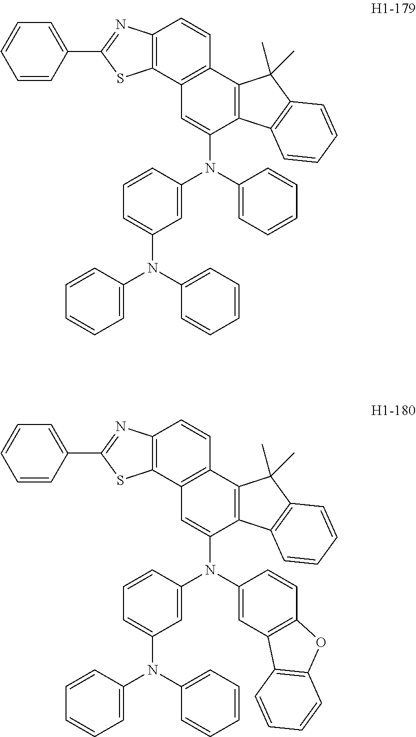 [SMILES: CC1(C)c2ccccc2-c2c(N(c3ccccc3)c3cccc(N(c4ccccc4)c4ccccc4)c3)cc3c(ccc4nc(-c5ccccc5)sc43)c21 ; CC1(C)c2ccccc2-c2c(N(c3cccc(N(c4ccccc4)c4ccccc4)c3)c3ccc4oc5ccccc5c4c3)cc3c(ccc4nc(-c5ccccc5)sc43)c21]

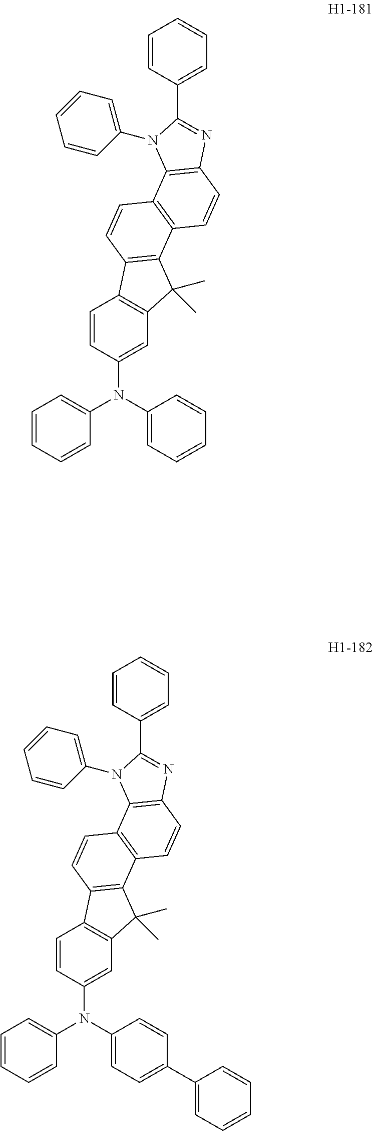 [SMILES: CC1(C)c2cc(N(c3ccccc3)c3ccccc3)ccc2-c2ccc3c(ccc4nc(-c5ccccc5)n(-c5ccccc5)c43)c21 ; CC1(C)c2cc(N(c3ccccc3)c3ccc(-c4ccccc4)cc3)ccc2-c2ccc3c(ccc4nc(-c5ccccc5)n(-c5ccccc5)c43)c21]

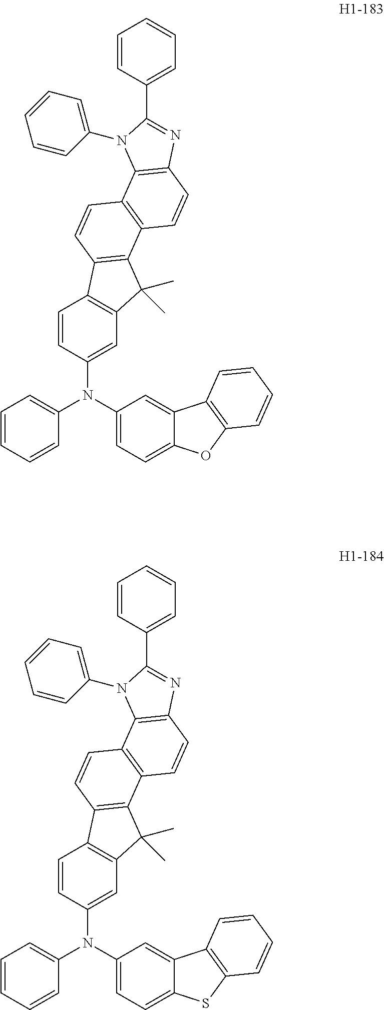 [SMILES: CC1(C)c2cc(N(c3ccccc3)c3ccc4oc5ccccc5c4c3)ccc2-c2ccc3c(ccc4nc(-c5ccccc5)n(-c5ccccc5)c43)c21 ; CC1(C)c2cc(N(c3ccccc3)c3ccc4sc5ccccc5c4c3)ccc2-c2ccc3c(ccc4nc(-c5ccccc5)n(-c5ccccc5)c43)c21]

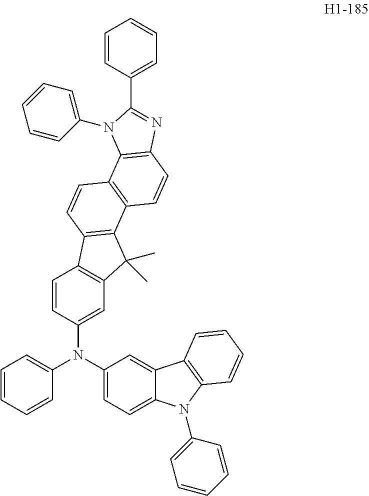 [SMILES: CC1(C)c2cc(N(c3ccccc3)c3ccc4c(c3)c3ccccc3n4-c3ccccc3)ccc2-c2ccc3c(ccc4nc(-c5ccccc5)n(-c5ccccc5)c43)c21]

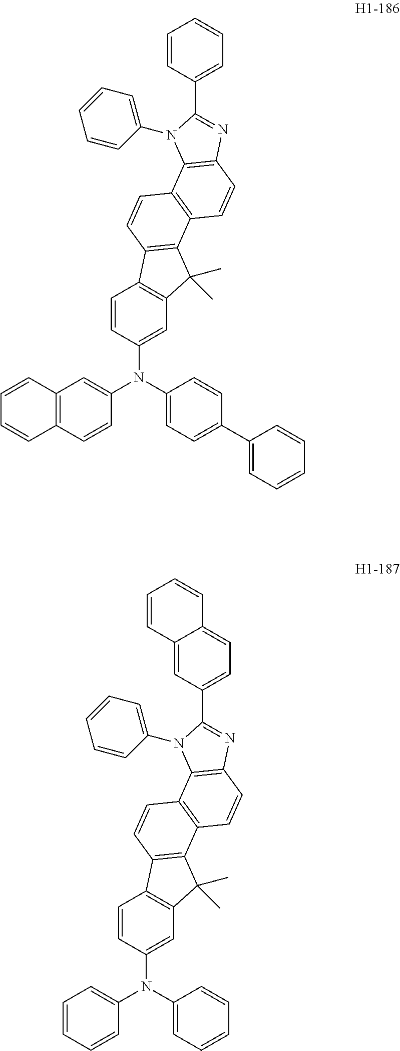 [SMILES: CC1(C)c2cc(N(c3ccc(-c4ccccc4)cc3)c3ccc4ccccc4c3)ccc2-c2ccc3c(ccc4nc(-c5ccccc5)n(-c5ccccc5)c43)c21 ; CC1(C)c2cc(N(c3ccccc3)c3ccccc3)ccc2-c2ccc3c(ccc4nc(-c5ccc6ccccc6c5)n(-c5ccccc5)c43)c21]

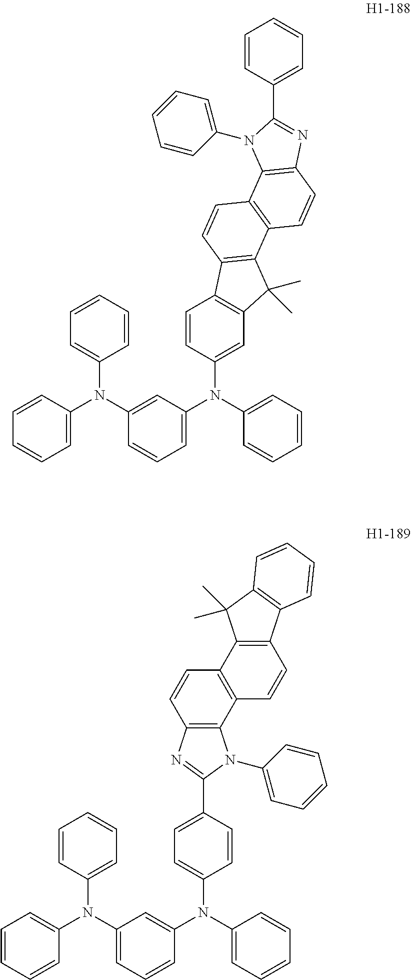 [SMILES: CC1(C)c2cc(N(c3ccccc3)c3cccc(N(c4ccccc4)c4ccccc4)c3)ccc2-c2ccc3c(ccc4nc(-c5ccccc5)n(-c5ccccc5)c43)c21 ; CC1(C)c2ccccc2-c2ccc3c(ccc4nc(-c5ccc(N(c6ccccc6)c6cccc(N(c7ccccc7)c7ccccc7)c6)cc5)n(-c5ccccc5)c43)c21]

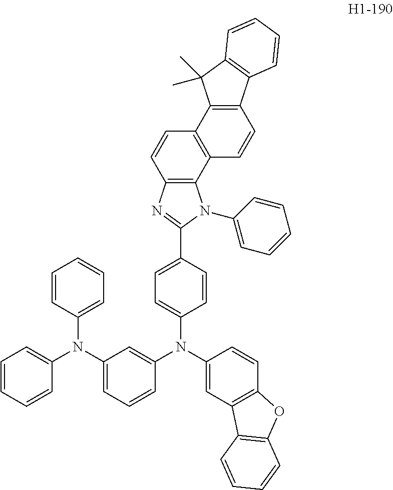 [SMILES: CC1(C)c2ccccc2-c2ccc3c(ccc4nc(-c5ccc(N(c6cccc(N(c7ccccc7)c7ccccc7)c6)c6ccc7oc8ccccc8c7c6)cc5)n(-c5ccccc5)c43)c21]

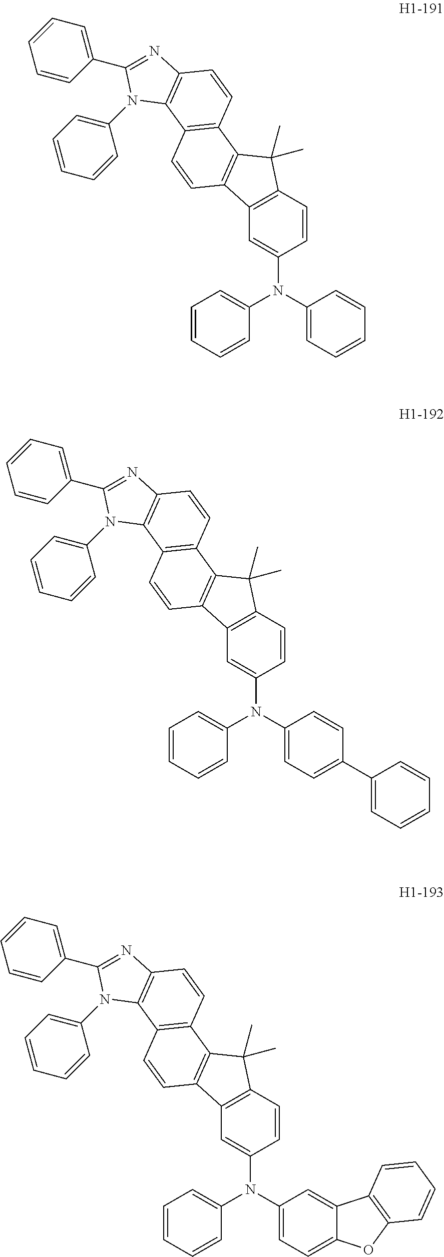 [SMILES: CC1(C)c2ccc(N(c3ccccc3)c3ccccc3)cc2-c2ccc3c(ccc4nc(-c5ccccc5)n(-c5ccccc5)c43)c21 ; CC1(C)c2ccc(N(c3ccccc3)c3ccc(-c4ccccc4)cc3)cc2-c2ccc3c(ccc4nc(-c5ccccc5)n(-c5ccccc5)c43)c21 ; CC1(C)c2ccc(N(c3ccccc3)c3ccc4oc5ccccc5c4c3)cc2-c2ccc3c(ccc4nc(-c5ccccc5)n(-c5ccccc5)c43)c21]

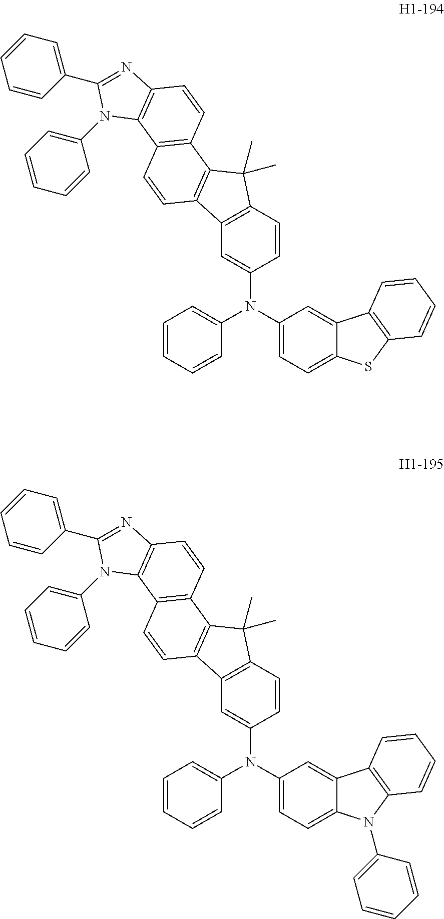 [SMILES: CC1(C)c2ccc(N(c3ccccc3)c3ccc4sc5ccccc5c4c3)cc2-c2ccc3c(ccc4nc(-c5ccccc5)n(-c5ccccc5)c43)c21 ; CC1(C)c2ccc(N(c3ccccc3)c3ccc4c(c3)c3ccccc3n4-c3ccccc3)cc2-c2ccc3c(ccc4nc(-c5ccccc5)n(-c5ccccc5)c43)c21]

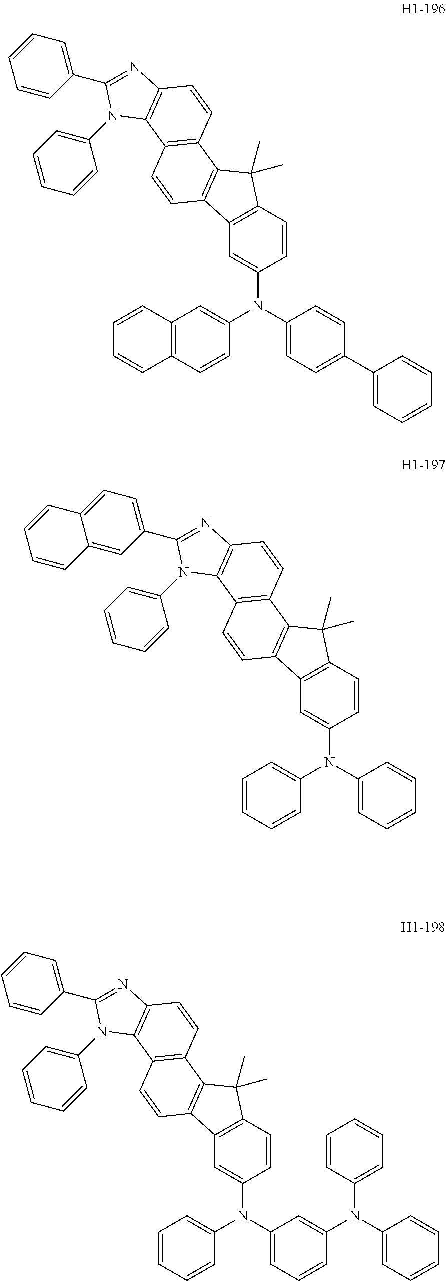 [SMILES: CC1(C)c2ccc(N(c3ccc(-c4ccccc4)cc3)c3ccc4ccccc4c3)cc2-c2ccc3c(ccc4nc(-c5ccccc5)n(-c5ccccc5)c43)c21 ; CC1(C)c2ccc(N(c3ccccc3)c3ccccc3)cc2-c2ccc3c(ccc4nc(-c5ccc6ccccc6c5)n(-c5ccccc5)c43)c21 ; CC1(C)c2ccc(N(c3ccccc3)c3cccc(N(c4ccccc4)c4ccccc4)c3)cc2-c2ccc3c(ccc4nc(-c5ccccc5)n(-c5ccccc5)c43)c21]

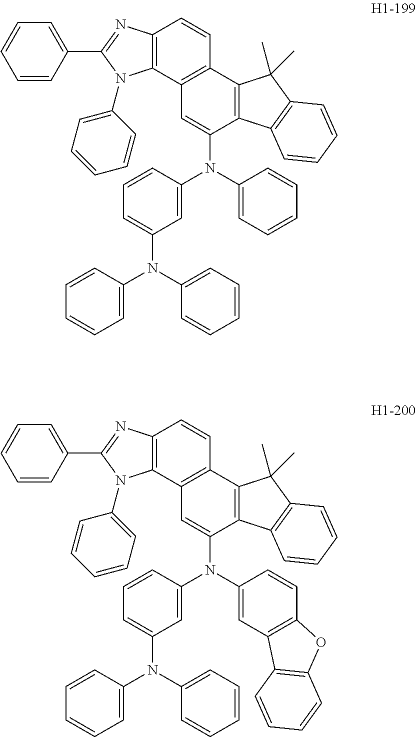 [SMILES: CC1(C)c2ccccc2-c2c(N(c3ccccc3)c3cccc(N(c4ccccc4)c4ccccc4)c3)cc3c(ccc4nc(-c5ccccc5)n(-c5ccccc5)c43)c21 ; CC1(C)c2ccccc2-c2c(N(c3cccc(N(c4ccccc4)c4ccccc4)c3)c3ccc4oc5ccccc5c4c3)cc3c(ccc4nc(-c5ccccc5)n(-c5ccccc5)c43)c21]

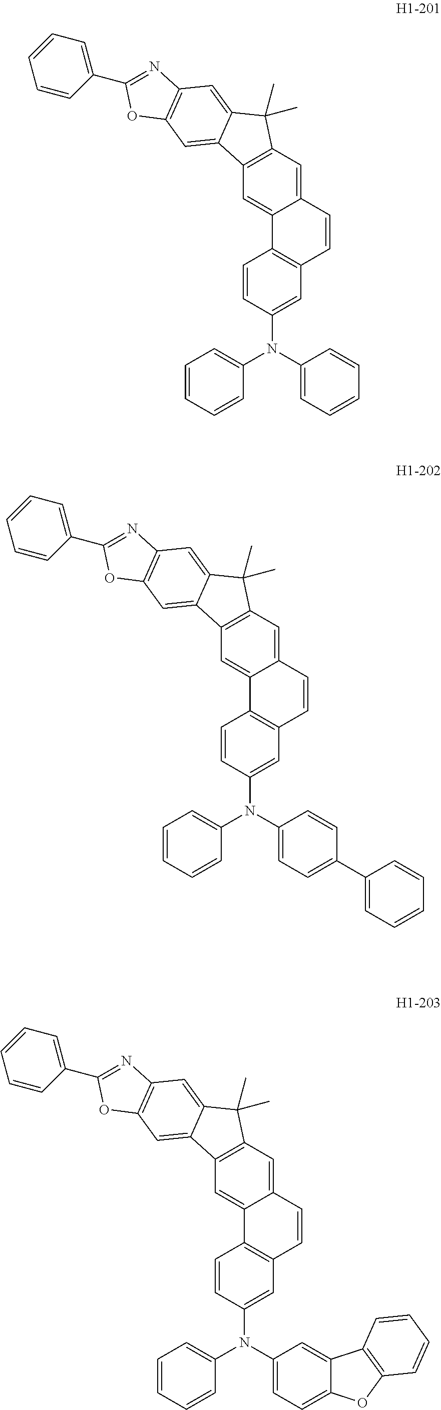 [SMILES: CC1(C)c2cc3ccc4cc(N(c5ccccc5)c5ccccc5)ccc4c3cc2-c2cc3oc(-c4ccccc4)nc3cc21 ; CC1(C)c2cc3ccc4cc(N(c5ccccc5)c5ccc(-c6ccccc6)cc5)ccc4c3cc2-c2cc3oc(-c4ccccc4)nc3cc21 ; CC1(C)c2cc3ccc4cc(N(c5ccccc5)c5ccc6oc7ccccc7c6c5)ccc4c3cc2-c2cc3oc(-c4ccccc4)nc3cc21]

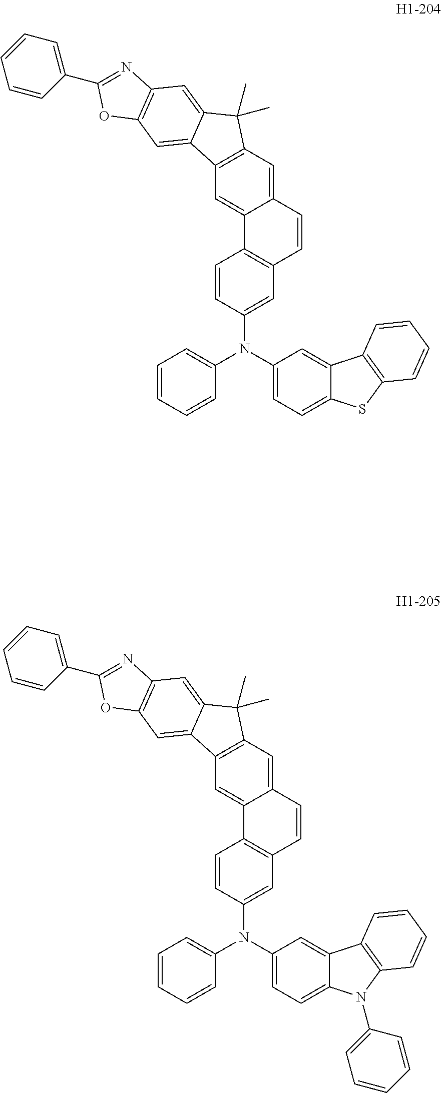 [SMILES: CC1(C)c2cc3ccc4cc(N(c5ccccc5)c5ccc6sc7ccccc7c6c5)ccc4c3cc2-c2cc3oc(-c4ccccc4)nc3cc21 ; CC1(C)c2cc3ccc4cc(N(c5ccccc5)c5ccc6c(c5)c5ccccc5n6-c5ccccc5)ccc4c3cc2-c2cc3oc(-c4ccccc4)nc3cc21]

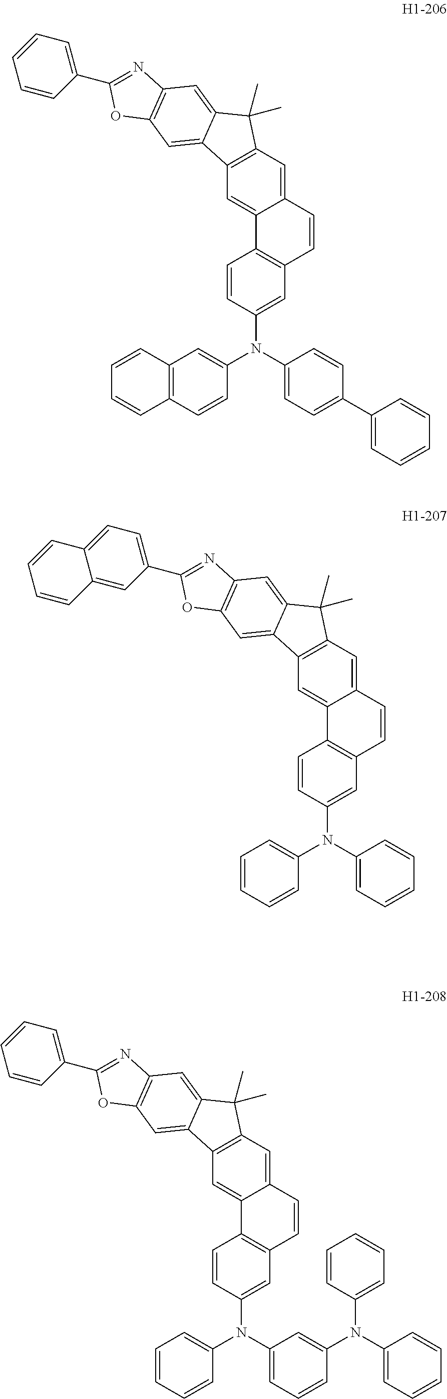 [SMILES: CC1(C)c2cc3ccc4cc(N(c5ccc(-c6ccccc6)cc5)c5ccc6ccccc6c5)ccc4c3cc2-c2cc3oc(-c4ccccc4)nc3cc21 ; CC1(C)c2cc3ccc4cc(N(c5ccccc5)c5ccccc5)ccc4c3cc2-c2cc3oc(-c4ccc5ccccc5c4)nc3cc21 ; CC1(C)c2cc3ccc4cc(N(c5ccccc5)c5cccc(N(c6ccccc6)c6ccccc6)c5)ccc4c3cc2-c2cc3oc(-c4ccccc4)nc3cc21]

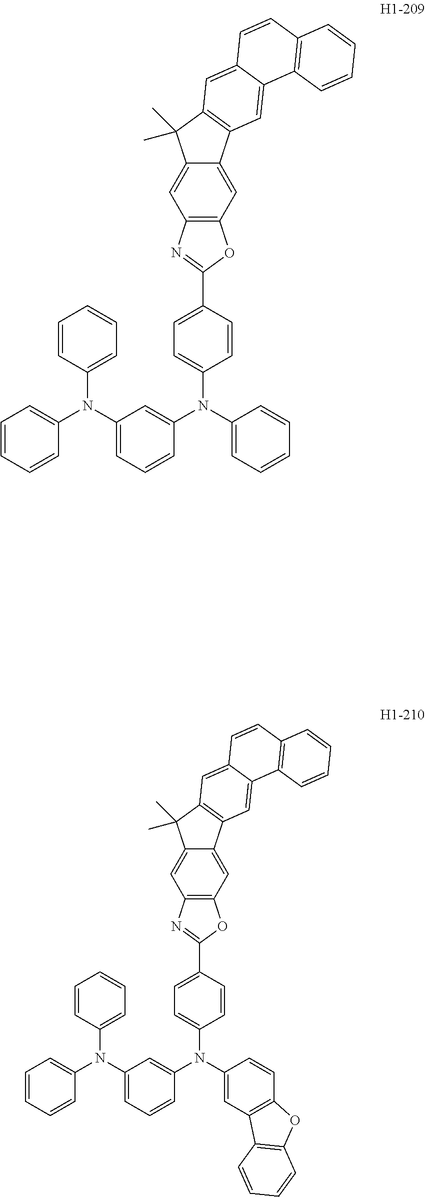 [SMILES: CC1(C)c2cc3ccc4ccccc4c3cc2-c2cc3oc(-c4ccc(N(c5ccccc5)c5cccc(N(c6ccccc6)c6ccccc6)c5)cc4)nc3cc21 ; CC1(C)c2cc3ccc4ccccc4c3cc2-c2cc3oc(-c4ccc(N(c5cccc(N(c6ccccc6)c6ccccc6)c5)c5ccc6oc7ccccc7c6c5)cc4)nc3cc21]

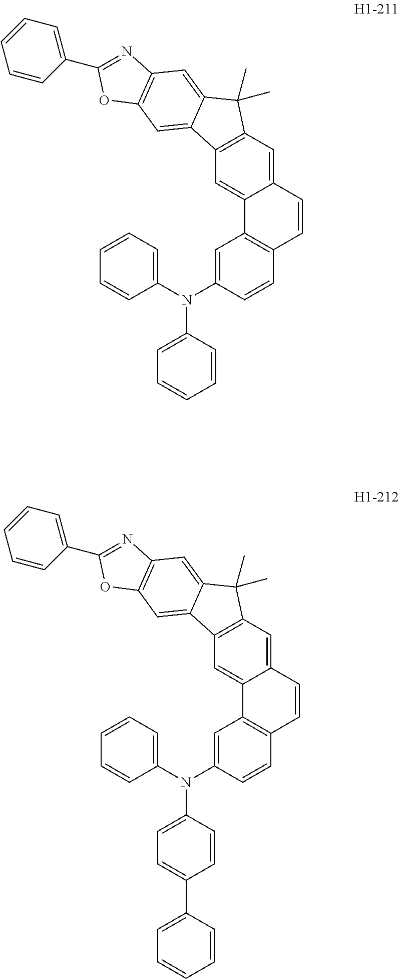 [SMILES: CC1(C)c2cc3ccc4ccc(N(c5ccccc5)c5ccccc5)cc4c3cc2-c2cc3oc(-c4ccccc4)nc3cc21 ; CC1(C)c2cc3ccc4ccc(N(c5ccccc5)c5ccc(-c6ccccc6)cc5)cc4c3cc2-c2cc3oc(-c4ccccc4)nc3cc21]

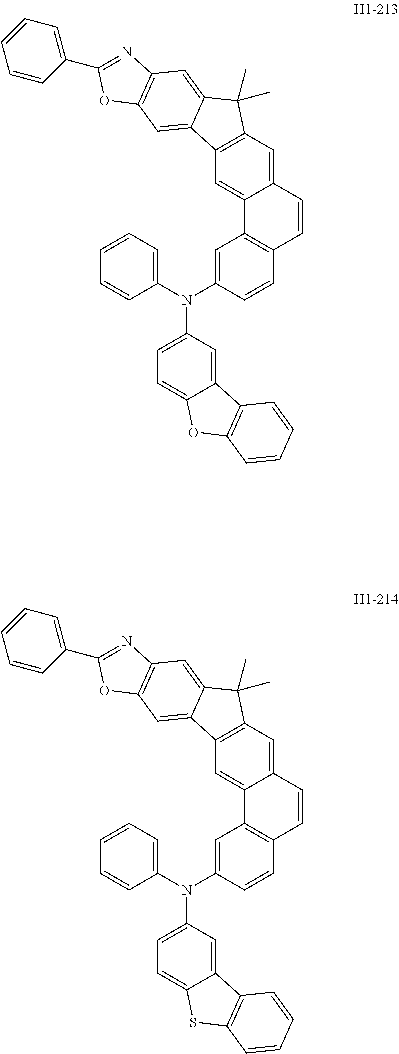 [SMILES: CC1(C)c2cc3ccc4ccc(N(c5ccccc5)c5ccc6oc7ccccc7c6c5)cc4c3cc2-c2cc3oc(-c4ccccc4)nc3cc21 ; CC1(C)c2cc3ccc4ccc(N(c5ccccc5)c5ccc6sc7ccccc7c6c5)cc4c3cc2-c2cc3oc(-c4ccccc4)nc3cc21]

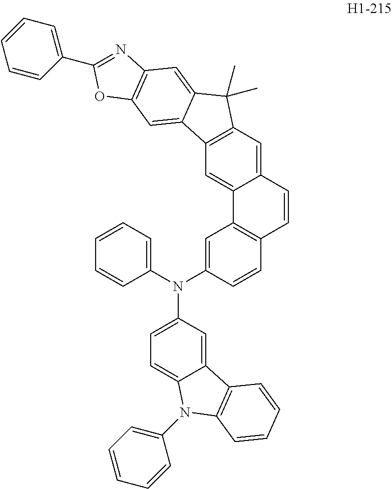 [SMILES: CC1(C)c2cc3ccc4ccc(N(c5ccccc5)c5ccc6c(c5)c5ccccc5n6-c5ccccc5)cc4c3cc2-c2cc3oc(-c4ccccc4)nc3cc21]

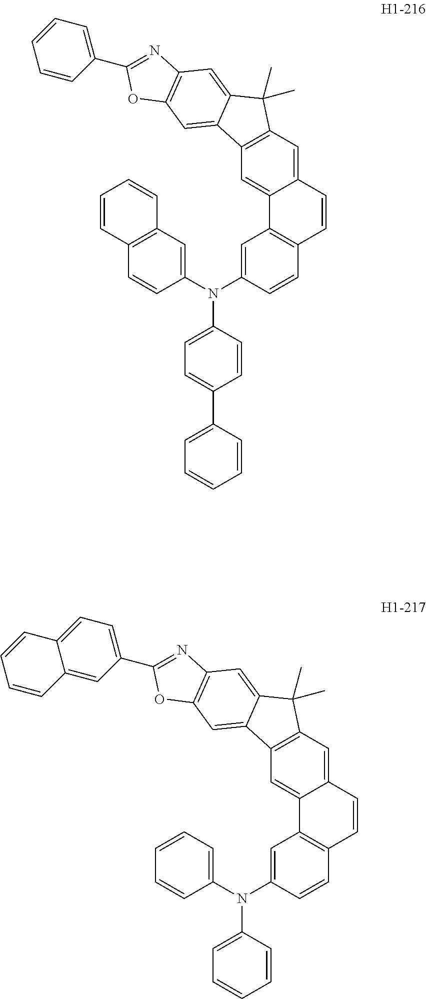 [SMILES: CC1(C)c2cc3ccc4ccc(N(c5ccc(-c6ccccc6)cc5)c5ccc6ccccc6c5)cc4c3cc2-c2cc3oc(-c4ccccc4)nc3cc21 ; CC1(C)c2cc3ccc4ccc(N(c5ccccc5)c5ccccc5)cc4c3cc2-c2cc3oc(-c4ccc5ccccc5c4)nc3cc21]

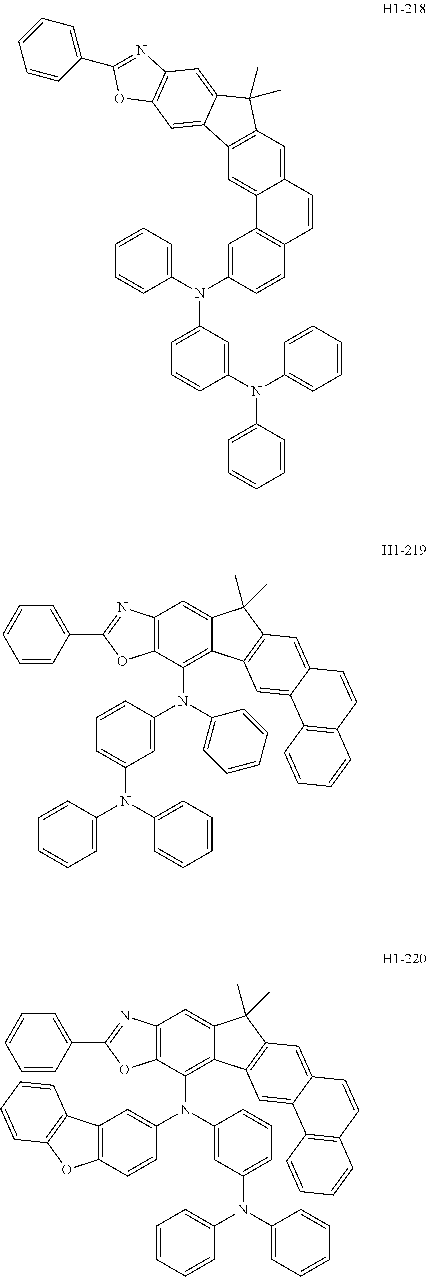 [SMILES: CC1(C)c2cc3ccc4ccc(N(c5ccccc5)c5cccc(N(c6ccccc6)c6ccccc6)c5)cc4c3cc2-c2cc3oc(-c4ccccc4)nc3cc21 ; CC1(C)c2cc3ccc4ccccc4c3cc2-c2c1cc1nc(-c3ccccc3)oc1c2N(c1ccccc1)c1cccc(N(c2ccccc2)c2ccccc2)c1 ; CC1(C)c2cc3ccc4ccccc4c3cc2-c2c1cc1nc(-c3ccccc3)oc1c2N(c1cccc(N(c2ccccc2)c2ccccc2)c1)c1ccc2oc3ccccc3c2c1]

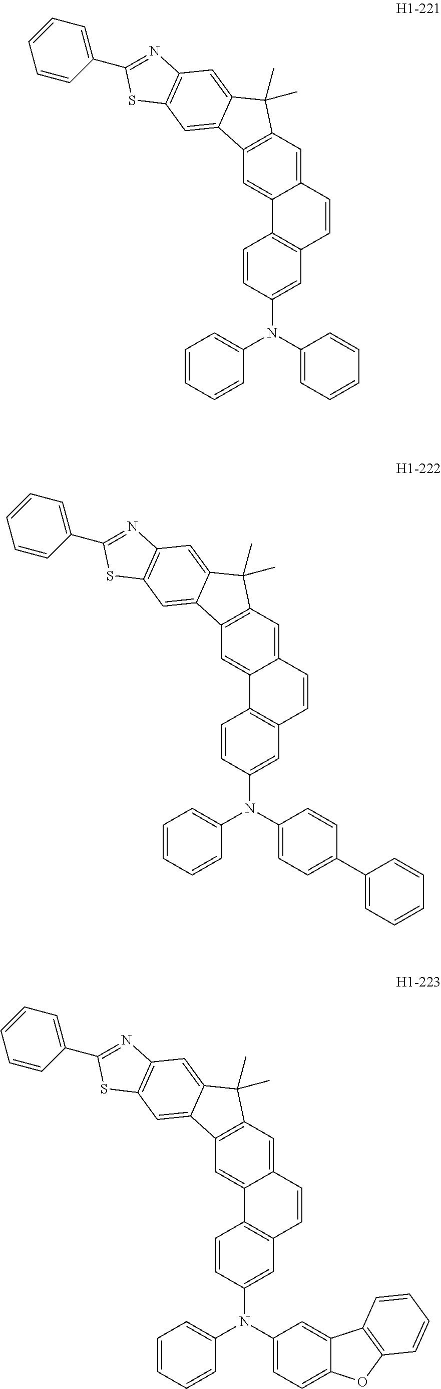 [SMILES: CC1(C)c2cc3ccc4cc(N(c5ccccc5)c5ccccc5)ccc4c3cc2-c2cc3sc(-c4ccccc4)nc3cc21 ; CC1(C)c2cc3ccc4cc(N(c5ccccc5)c5ccc(-c6ccccc6)cc5)ccc4c3cc2-c2cc3sc(-c4ccccc4)nc3cc21 ; CC1(C)c2cc3ccc4cc(N(c5ccccc5)c5ccc6oc7ccccc7c6c5)ccc4c3cc2-c2cc3sc(-c4ccccc4)nc3cc21]

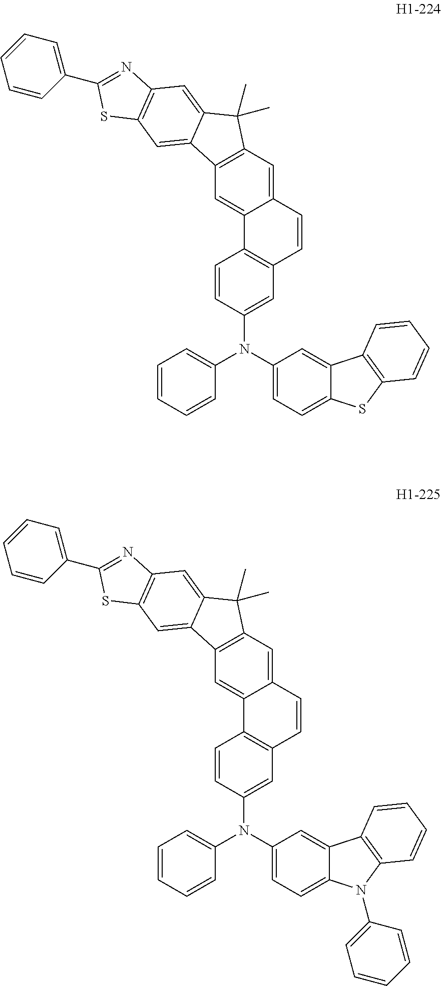 [SMILES: CC1(C)c2cc3ccc4cc(N(c5ccccc5)c5ccc6sc7ccccc7c6c5)ccc4c3cc2-c2cc3sc(-c4ccccc4)nc3cc21 ; CC1(C)c2cc3ccc4cc(N(c5ccccc5)c5ccc6c(c5)c5ccccc5n6-c5ccccc5)ccc4c3cc2-c2cc3sc(-c4ccccc4)nc3cc21]

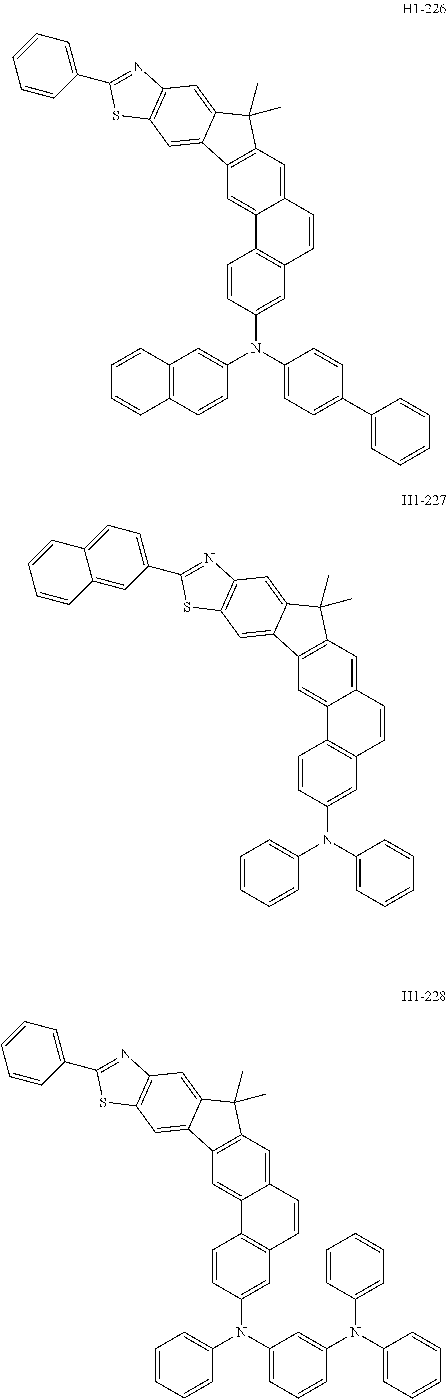 [SMILES: CC1(C)c2cc3ccc4cc(N(c5ccc(-c6ccccc6)cc5)c5ccc6ccccc6c5)ccc4c3cc2-c2cc3sc(-c4ccccc4)nc3cc21 ; CC1(C)c2cc3ccc4cc(N(c5ccccc5)c5ccccc5)ccc4c3cc2-c2cc3sc(-c4ccc5ccccc5c4)nc3cc21 ; CC1(C)c2cc3ccc4cc(N(c5ccccc5)c5cccc(N(c6ccccc6)c6ccccc6)c5)ccc4c3cc2-c2cc3sc(-c4ccccc4)nc3cc21]

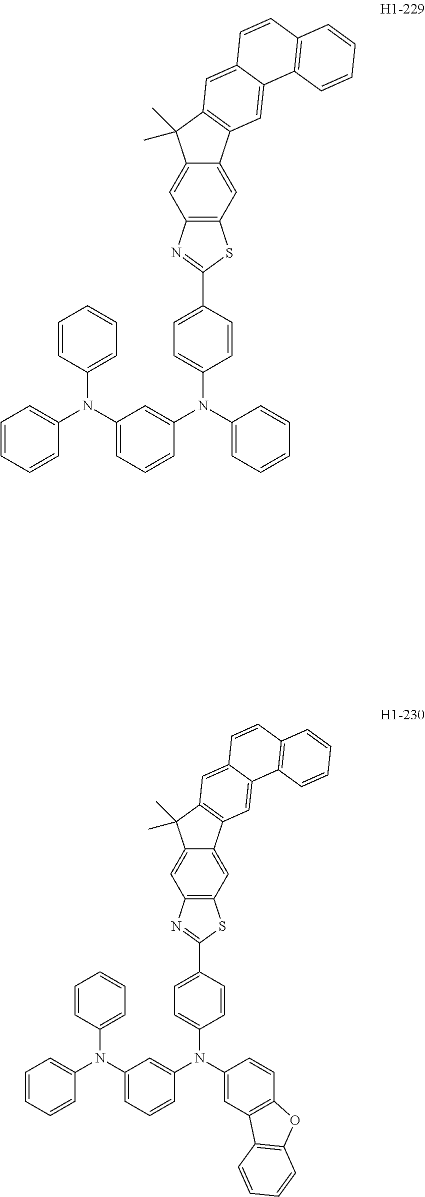 [SMILES: CC1(C)c2cc3ccc4ccccc4c3cc2-c2cc3sc(-c4ccc(N(c5ccccc5)c5cccc(N(c6ccccc6)c6ccccc6)c5)cc4)nc3cc21 ; CC1(C)c2cc3ccc4ccccc4c3cc2-c2cc3sc(-c4ccc(N(c5cccc(N(c6ccccc6)c6ccccc6)c5)c5ccc6oc7ccccc7c6c5)cc4)nc3cc21]

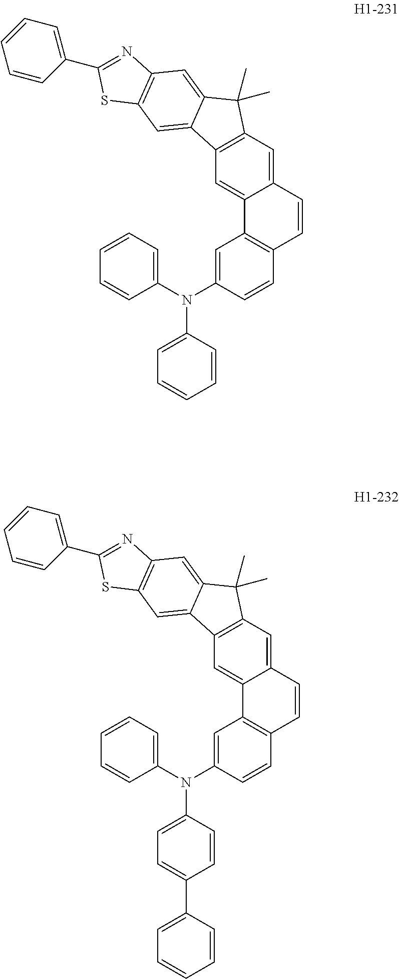 [SMILES: CC1(C)c2cc3ccc4ccc(N(c5ccccc5)c5ccccc5)cc4c3cc2-c2cc3sc(-c4ccccc4)nc3cc21 ; CC1(C)c2cc3ccc4ccc(N(c5ccccc5)c5ccc(-c6ccccc6)cc5)cc4c3cc2-c2cc3sc(-c4ccccc4)nc3cc21]

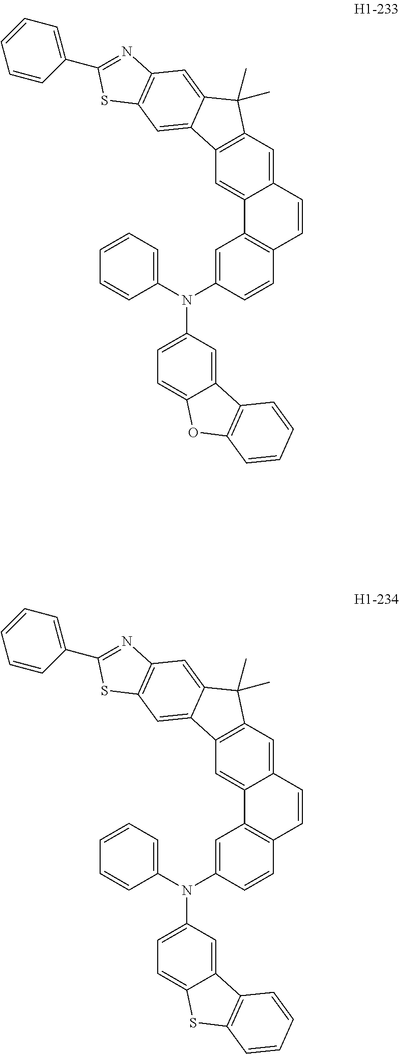 [SMILES: CC1(C)c2cc3ccc4ccc(N(c5ccccc5)c5ccc6oc7ccccc7c6c5)cc4c3cc2-c2cc3sc(-c4ccccc4)nc3cc21 ; CC1(C)c2cc3ccc4ccc(N(c5ccccc5)c5ccc6sc7ccccc7c6c5)cc4c3cc2-c2cc3sc(-c4ccccc4)nc3cc21]

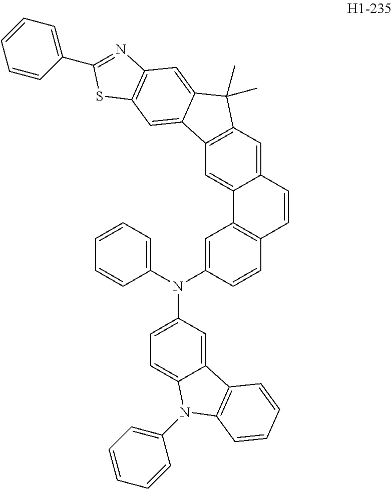 [SMILES: CC1(C)c2cc3ccc4ccc(N(c5ccccc5)c5ccc6c(c5)c5ccccc5n6-c5ccccc5)cc4c3cc2-c2cc3sc(-c4ccccc4)nc3cc21]

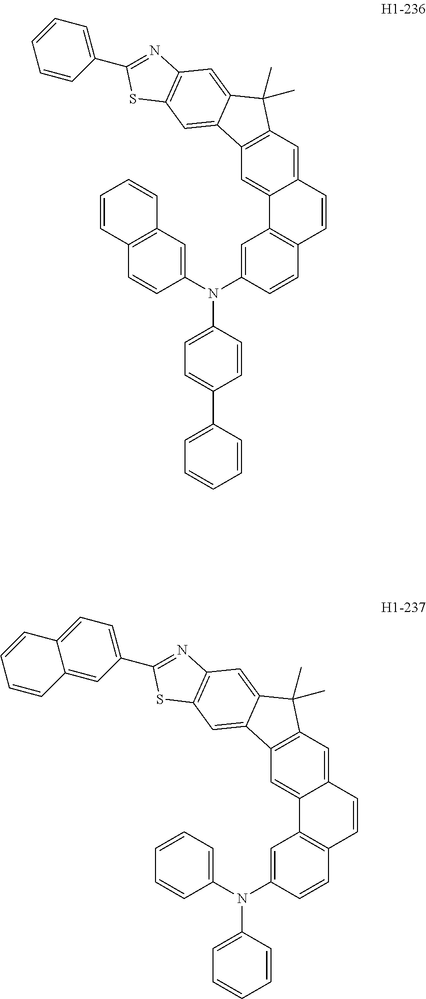 [SMILES: CC1(C)c2cc3ccc4ccc(N(c5ccc(-c6ccccc6)cc5)c5ccc6ccccc6c5)cc4c3cc2-c2cc3sc(-c4ccccc4)nc3cc21 ; CC1(C)c2cc3ccc4ccc(N(c5ccccc5)c5ccccc5)cc4c3cc2-c2cc3sc(-c4ccc5ccccc5c4)nc3cc21]

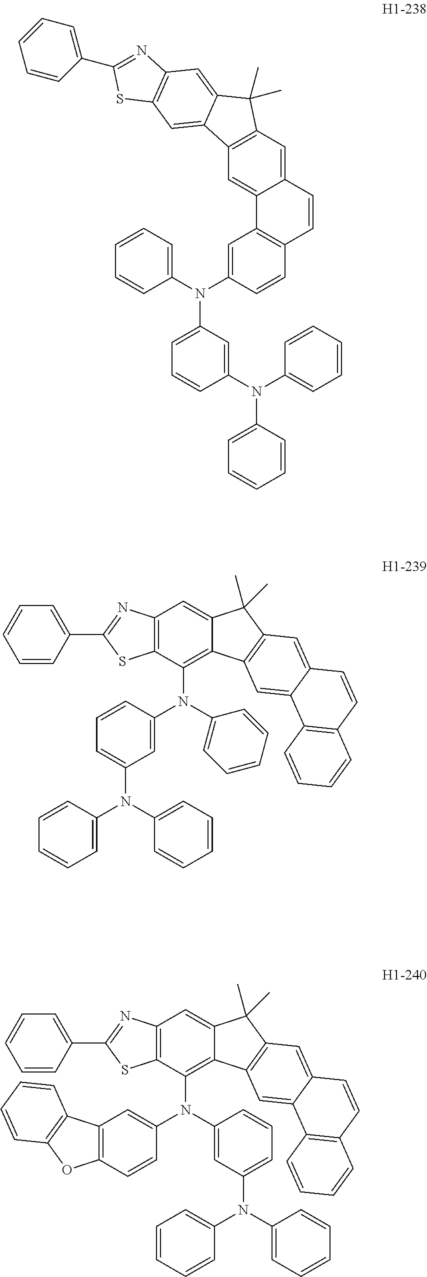 [SMILES: CC1(C)c2cc3ccc4ccc(N(c5ccccc5)c5cccc(N(c6ccccc6)c6ccccc6)c5)cc4c3cc2-c2cc3sc(-c4ccccc4)nc3cc21 ; CC1(C)c2cc3ccc4ccccc4c3cc2-c2c1cc1nc(-c3ccccc3)sc1c2N(c1ccccc1)c1cccc(N(c2ccccc2)c2ccccc2)c1 ; CC1(C)c2cc3ccc4ccccc4c3cc2-c2c1cc1nc(-c3ccccc3)sc1c2N(c1cccc(N(c2ccccc2)c2ccccc2)c1)c1ccc2oc3ccccc3c2c1]

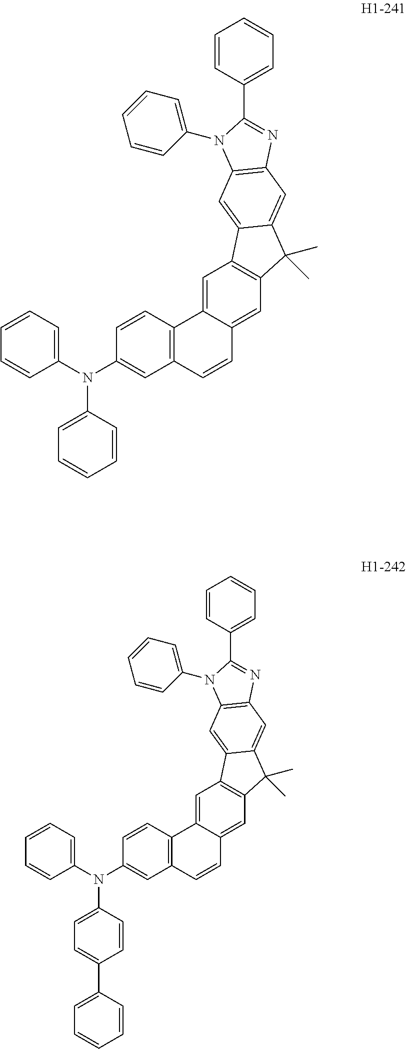 [SMILES: CC1(C)c2cc3ccc4cc(N(c5ccccc5)c5ccccc5)ccc4c3cc2-c2cc3c(cc21)nc(-c1ccccc1)n3-c1ccccc1 ; CC1(C)c2cc3ccc4cc(N(c5ccccc5)c5ccc(-c6ccccc6)cc5)ccc4c3cc2-c2cc3c(cc21)nc(-c1ccccc1)n3-c1ccccc1]

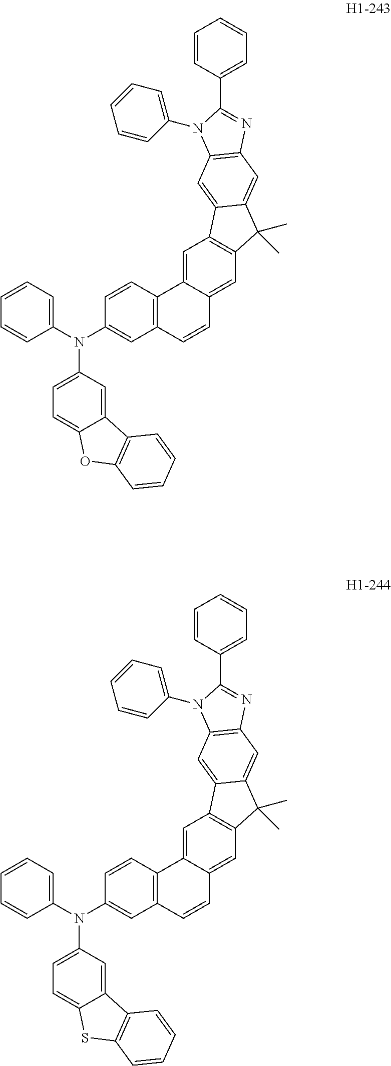 [SMILES: CC1(C)c2cc3ccc4cc(N(c5ccccc5)c5ccc6oc7ccccc7c6c5)ccc4c3cc2-c2cc3c(cc21)nc(-c1ccccc1)n3-c1ccccc1 ; CC1(C)c2cc3ccc4cc(N(c5ccccc5)c5ccc6sc7ccccc7c6c5)ccc4c3cc2-c2cc3c(cc21)nc(-c1ccccc1)n3-c1ccccc1]

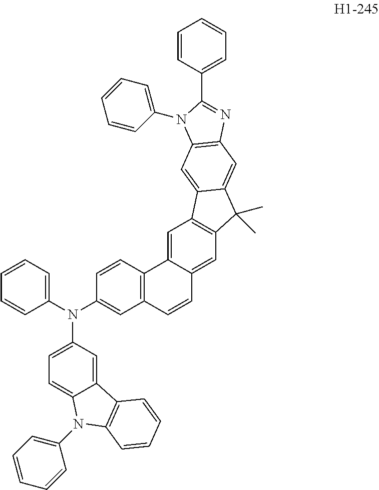 [SMILES: CC1(C)c2cc3ccc4cc(N(c5ccccc5)c5ccc6c(c5)c5ccccc5n6-c5ccccc5)ccc4c3cc2-c2cc3c(cc21)nc(-c1ccccc1)n3-c1ccccc1]

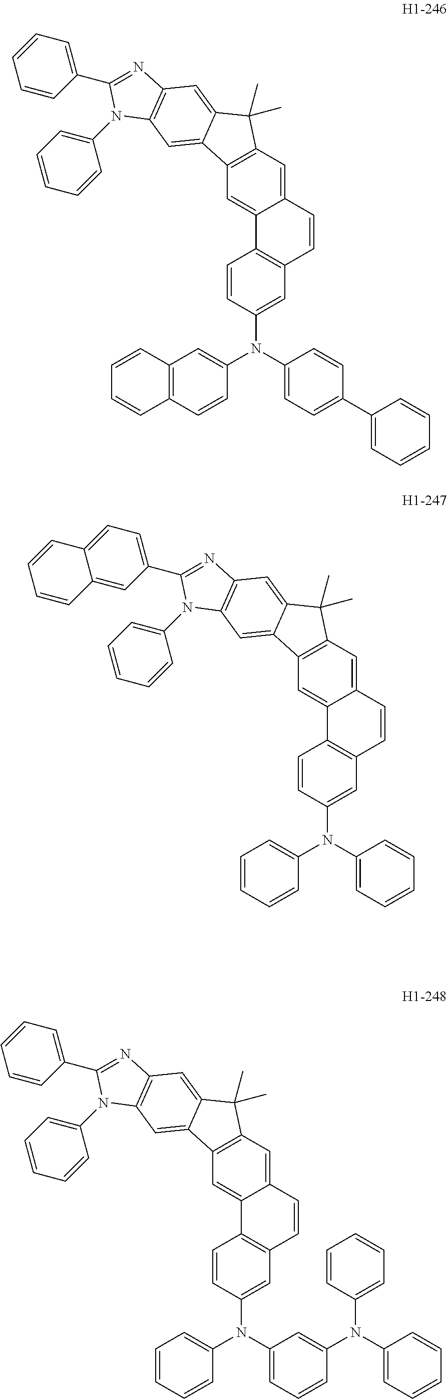 [SMILES: CC1(C)c2cc3ccc4cc(N(c5ccc(-c6ccccc6)cc5)c5ccc6ccccc6c5)ccc4c3cc2-c2cc3c(cc21)nc(-c1ccccc1)n3-c1ccccc1 ; CC1(C)c2cc3ccc4cc(N(c5ccccc5)c5ccccc5)ccc4c3cc2-c2cc3c(cc21)nc(-c1ccc2ccccc2c1)n3-c1ccccc1 ; CC1(C)c2cc3ccc4cc(N(c5ccccc5)c5cccc(N(c6ccccc6)c6ccccc6)c5)ccc4c3cc2-c2cc3c(cc21)nc(-c1ccccc1)n3-c1ccccc1]

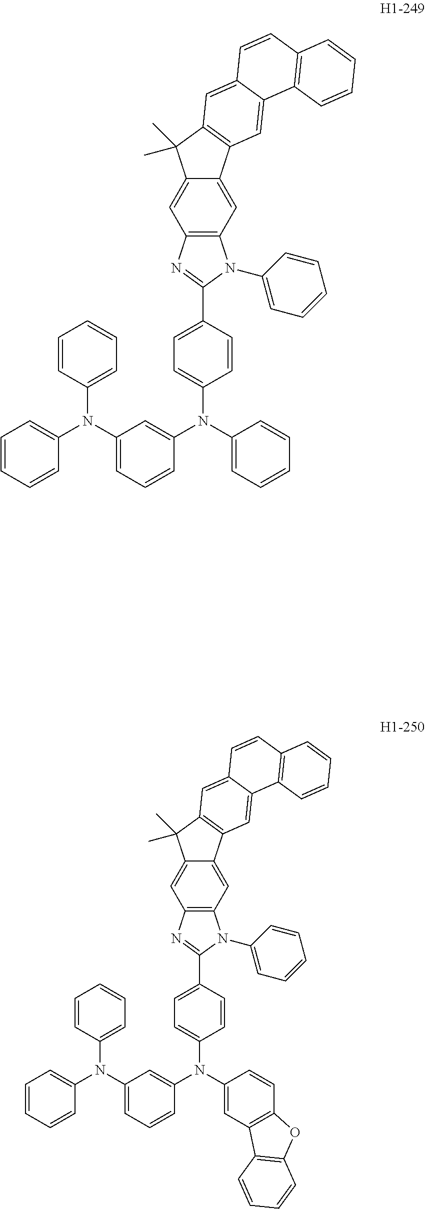 [SMILES: CC1(C)c2cc3ccc4ccccc4c3cc2-c2cc3c(cc21)nc(-c1ccc(N(c2ccccc2)c2cccc(N(c4ccccc4)c4ccccc4)c2)cc1)n3-c1ccccc1 ; CC1(C)c2cc3ccc4ccccc4c3cc2-c2cc3c(cc21)nc(-c1ccc(N(c2cccc(N(c4ccccc4)c4ccccc4)c2)c2ccc4oc5ccccc5c4c2)cc1)n3-c1ccccc1]

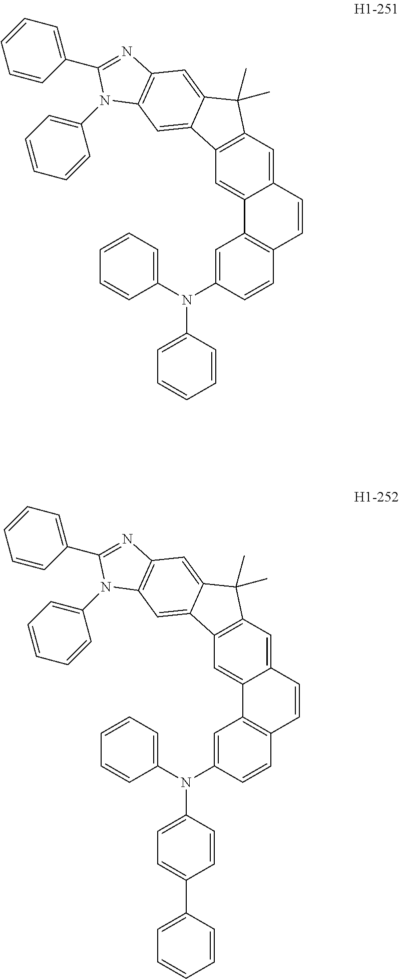 [SMILES: CC1(C)c2cc3ccc4ccc(N(c5ccccc5)c5ccccc5)cc4c3cc2-c2cc3c(cc21)nc(-c1ccccc1)n3-c1ccccc1 ; CC1(C)c2cc3ccc4ccc(N(c5ccccc5)c5ccc(-c6ccccc6)cc5)cc4c3cc2-c2cc3c(cc21)nc(-c1ccccc1)n3-c1ccccc1]

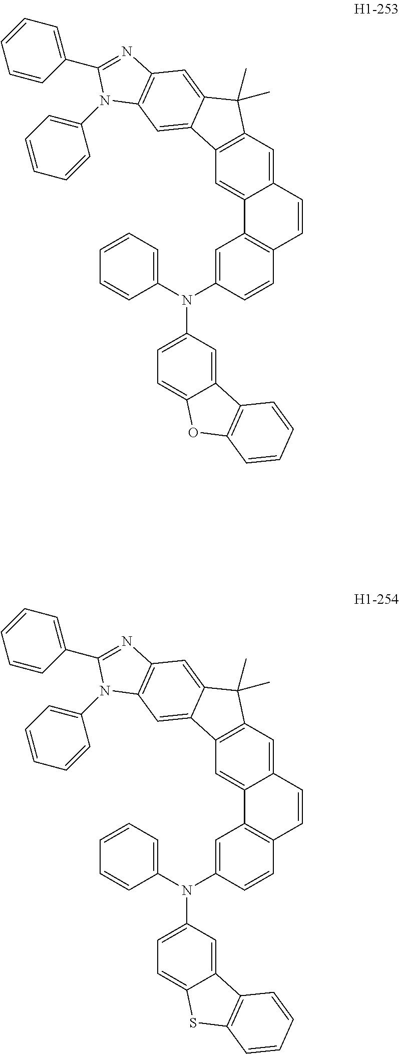 [SMILES: CC1(C)c2cc3ccc4ccc(N(c5ccccc5)c5ccc6oc7ccccc7c6c5)cc4c3cc2-c2cc3c(cc21)nc(-c1ccccc1)n3-c1ccccc1 ; CC1(C)c2cc3ccc4ccc(N(c5ccccc5)c5ccc6sc7ccccc7c6c5)cc4c3cc2-c2cc3c(cc21)nc(-c1ccccc1)n3-c1ccccc1]

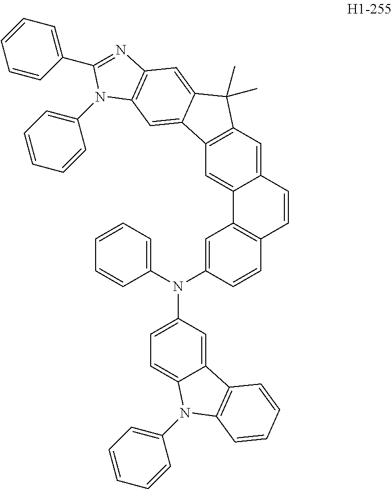 [SMILES: CC1(C)c2cc3ccc4ccc(N(c5ccccc5)c5ccc6c(c5)c5ccccc5n6-c5ccccc5)cc4c3cc2-c2cc3c(cc21)nc(-c1ccccc1)n3-c1ccccc1]

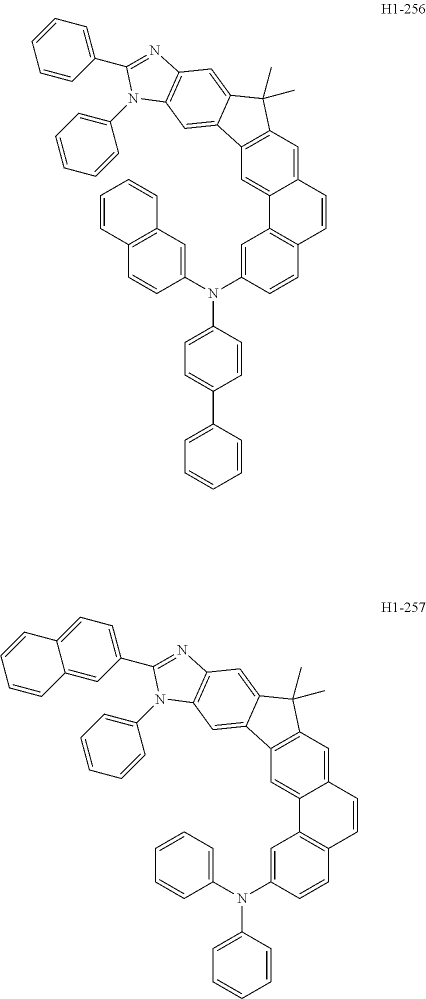 [SMILES: CC1(C)c2cc3ccc4ccc(N(c5ccc(-c6ccccc6)cc5)c5ccc6ccccc6c5)cc4c3cc2-c2cc3c(cc21)nc(-c1ccccc1)n3-c1ccccc1 ; CC1(C)c2cc3ccc4ccc(N(c5ccccc5)c5ccccc5)cc4c3cc2-c2cc3c(cc21)nc(-c1ccc2ccccc2c1)n3-c1ccccc1]

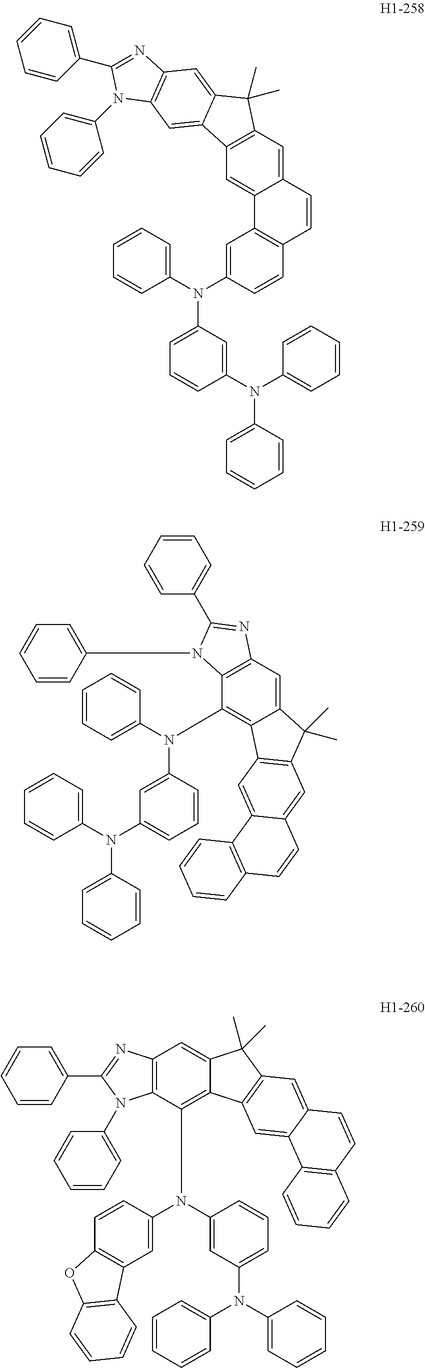 [SMILES: CC1(C)c2cc3ccc4ccc(N(c5ccccc5)c5cccc(N(c6ccccc6)c6ccccc6)c5)cc4c3cc2-c2cc3c(cc21)nc(-c1ccccc1)n3-c1ccccc1 ; CC1(C)c2cc3ccc4ccccc4c3cc2-c2c1cc1nc(-c3ccccc3)n(-c3ccccc3)c1c2N(c1ccccc1)c1cccc(N(c2ccccc2)c2ccccc2)c1 ; CC1(C)c2cc3ccc4ccccc4c3cc2-c2c1cc1nc(-c3ccccc3)n(-c3ccccc3)c1c2N(c1cccc(N(c2ccccc2)c2ccccc2)c1)c1ccc2oc3ccccc3c2c1]

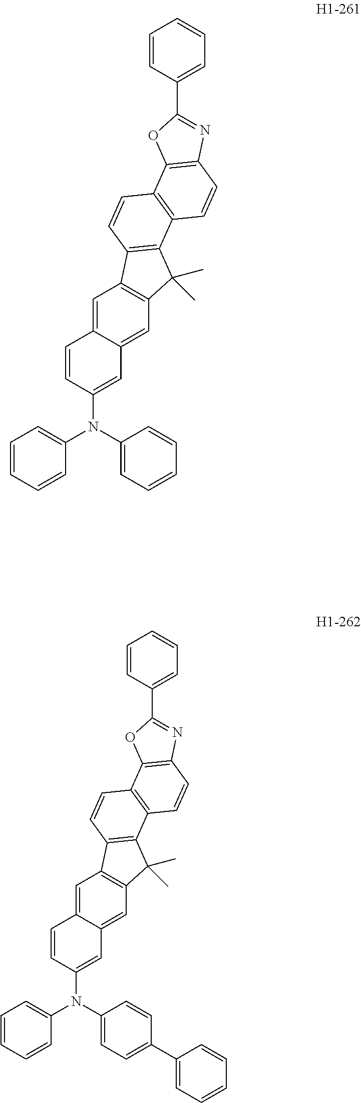 [SMILES: CC1(C)c2cc3cc(N(c4ccccc4)c4ccccc4)ccc3cc2-c2ccc3c(ccc4nc(-c5ccccc5)oc43)c21 ; CC1(C)c2cc3cc(N(c4ccccc4)c4ccc(-c5ccccc5)cc4)ccc3cc2-c2ccc3c(ccc4nc(-c5ccccc5)oc43)c21]

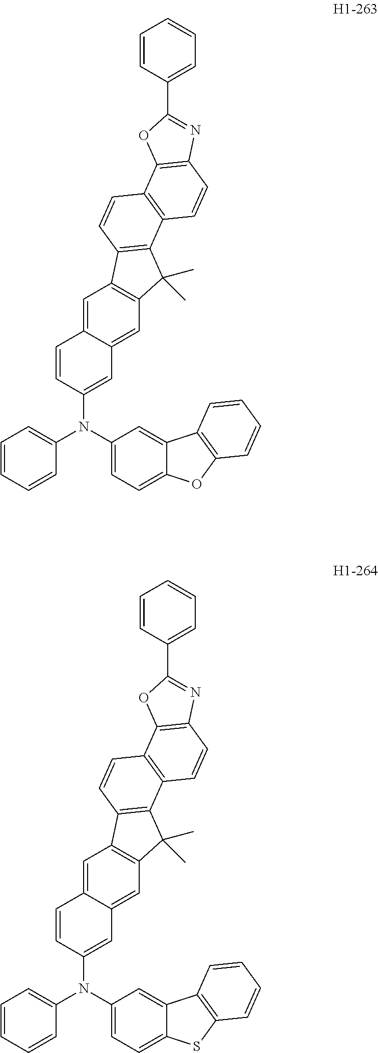 [SMILES: CC1(C)c2cc3cc(N(c4ccccc4)c4ccc5oc6ccccc6c5c4)ccc3cc2-c2ccc3c(ccc4nc(-c5ccccc5)oc43)c21 ; CC1(C)c2cc3cc(N(c4ccccc4)c4ccc5sc6ccccc6c5c4)ccc3cc2-c2ccc3c(ccc4nc(-c5ccccc5)oc43)c21]

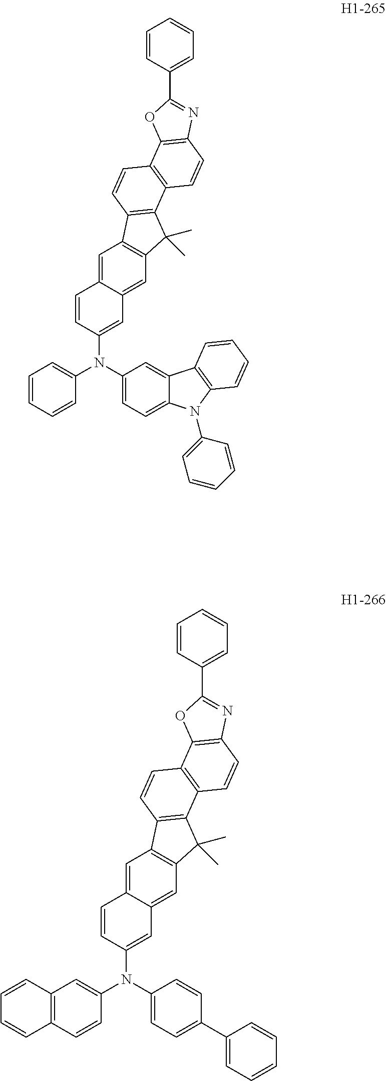 [SMILES: CC1(C)c2cc3cc(N(c4ccccc4)c4ccc5c(c4)c4ccccc4n5-c4ccccc4)ccc3cc2-c2ccc3c(ccc4nc(-c5ccccc5)oc43)c21 ; CC1(C)c2cc3cc(N(c4ccc(-c5ccccc5)cc4)c4ccc5ccccc5c4)ccc3cc2-c2ccc3c(ccc4nc(-c5ccccc5)oc43)c21]

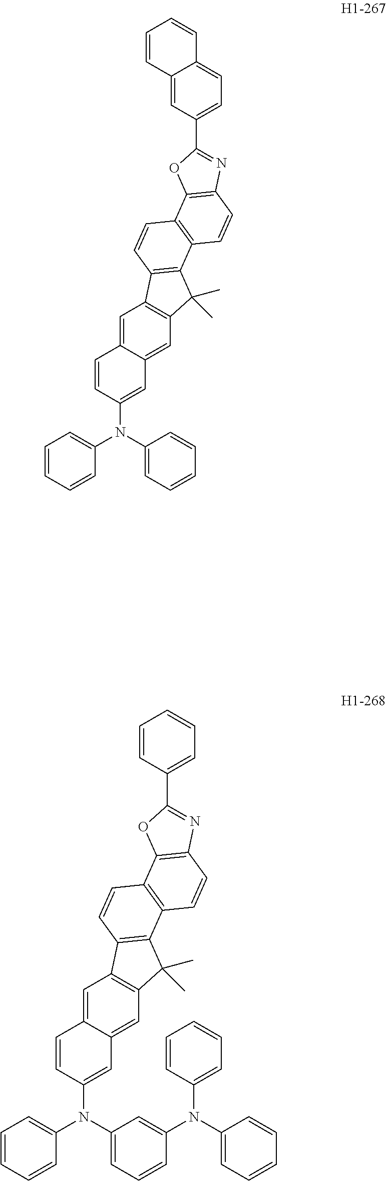 [SMILES: CC1(C)c2cc3cc(N(c4ccccc4)c4ccccc4)ccc3cc2-c2ccc3c(ccc4nc(-c5ccc6ccccc6c5)oc43)c21 ; CC1(C)c2cc3cc(N(c4ccccc4)c4cccc(N(c5ccccc5)c5ccccc5)c4)ccc3cc2-c2ccc3c(ccc4nc(-c5ccccc5)oc43)c21]

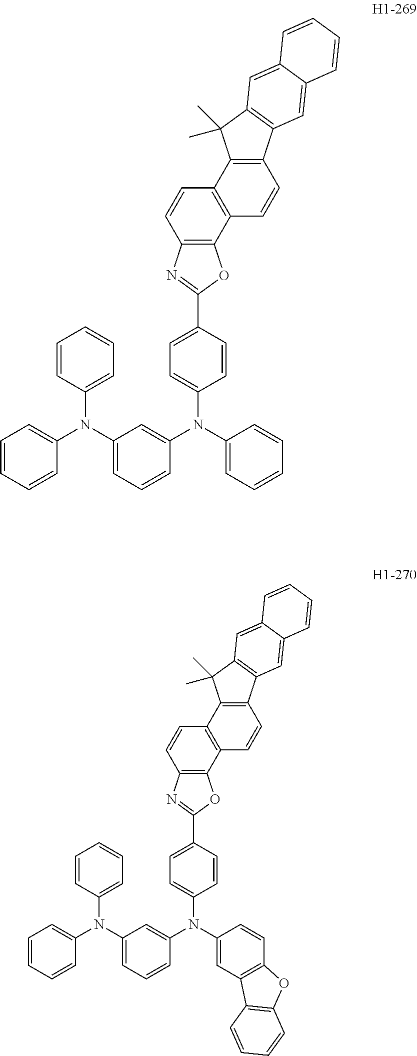 [SMILES: CC1(C)c2cc3ccccc3cc2-c2ccc3c(ccc4nc(-c5ccc(N(c6ccccc6)c6cccc(N(c7ccccc7)c7ccccc7)c6)cc5)oc43)c21 ; CC1(C)c2cc3ccccc3cc2-c2ccc3c(ccc4nc(-c5ccc(N(c6cccc(N(c7ccccc7)c7ccccc7)c6)c6ccc7oc8ccccc8c7c6)cc5)oc43)c21]

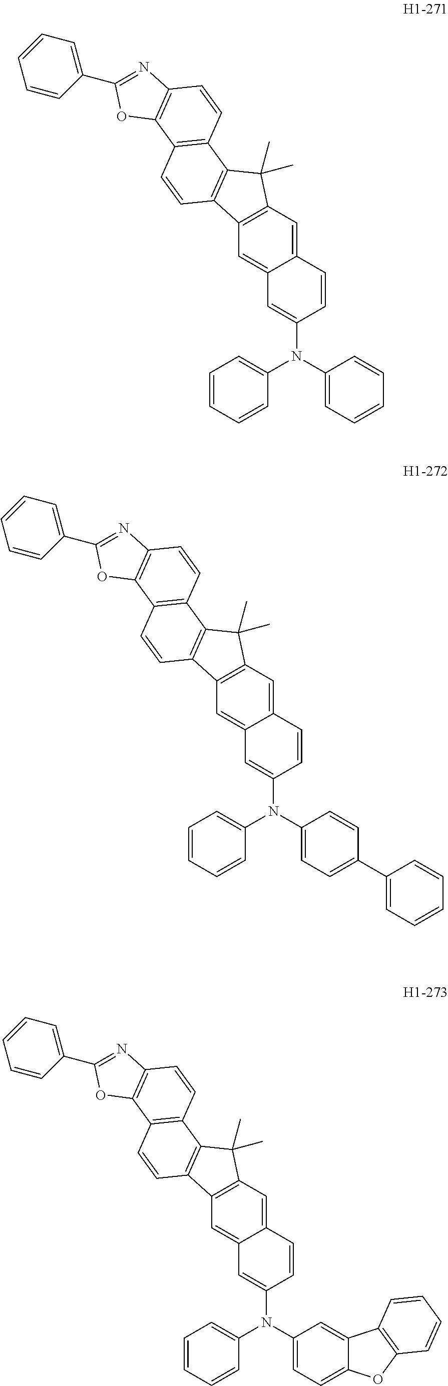 [SMILES: CC1(C)c2cc3ccc(N(c4ccccc4)c4ccccc4)cc3cc2-c2ccc3c(ccc4nc(-c5ccccc5)oc43)c21 ; CC1(C)c2cc3ccc(N(c4ccccc4)c4ccc(-c5ccccc5)cc4)cc3cc2-c2ccc3c(ccc4nc(-c5ccccc5)oc43)c21 ; CC1(C)c2cc3ccc(N(c4ccccc4)c4ccc5oc6ccccc6c5c4)cc3cc2-c2ccc3c(ccc4nc(-c5ccccc5)oc43)c21]

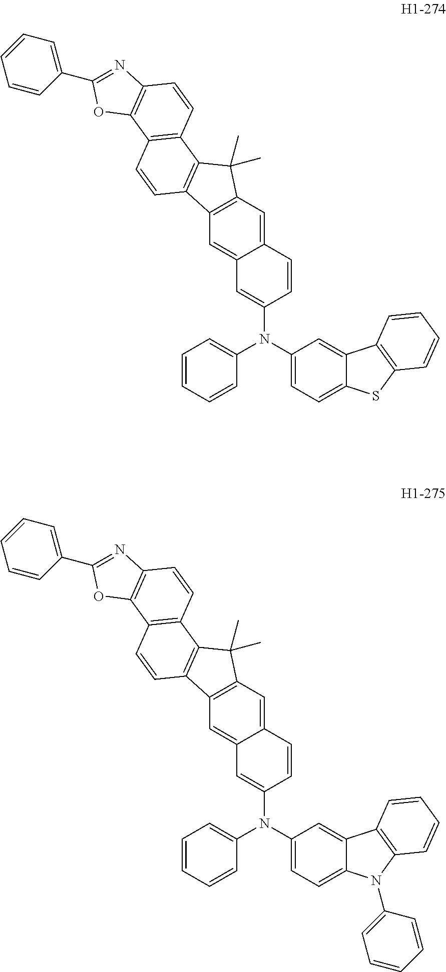 [SMILES: CC1(C)c2cc3ccc(N(c4ccccc4)c4ccc5sc6ccccc6c5c4)cc3cc2-c2ccc3c(ccc4nc(-c5ccccc5)oc43)c21 ; CC1(C)c2cc3ccc(N(c4ccccc4)c4ccc5c(c4)c4ccccc4n5-c4ccccc4)cc3cc2-c2ccc3c(ccc4nc(-c5ccccc5)oc43)c21]

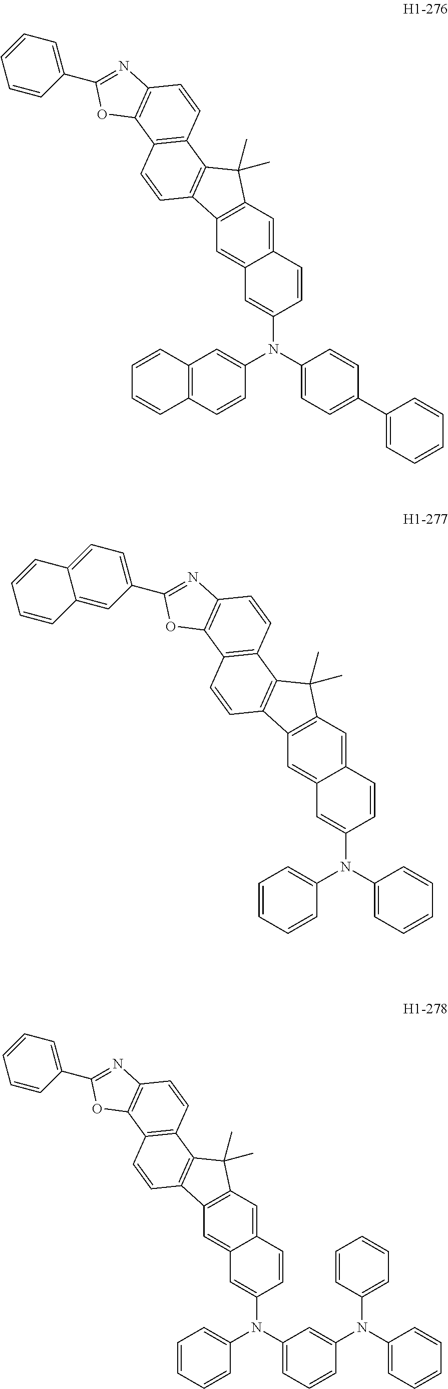 [SMILES: CC1(C)c2cc3ccc(N(c4ccc(-c5ccccc5)cc4)c4ccc5ccccc5c4)cc3cc2-c2ccc3c(ccc4nc(-c5ccccc5)oc43)c21 ; CC1(C)c2cc3ccc(N(c4ccccc4)c4ccccc4)cc3cc2-c2ccc3c(ccc4nc(-c5ccc6ccccc6c5)oc43)c21 ; CC1(C)c2cc3ccc(N(c4ccccc4)c4cccc(N(c5ccccc5)c5ccccc5)c4)cc3cc2-c2ccc3c(ccc4nc(-c5ccccc5)oc43)c21]

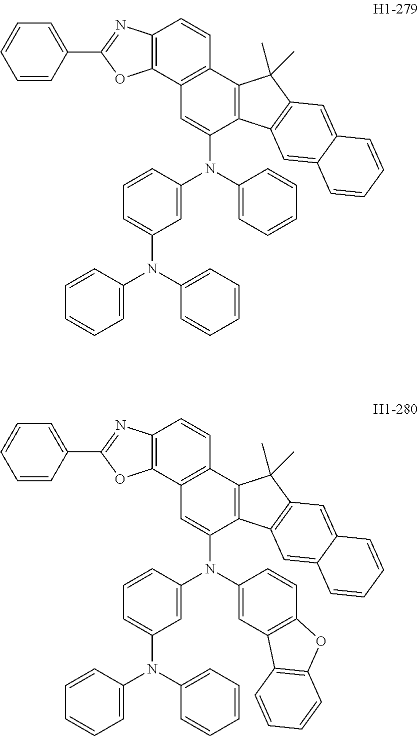 [SMILES: CC1(C)c2cc3ccccc3cc2-c2c(N(c3ccccc3)c3cccc(N(c4ccccc4)c4ccccc4)c3)cc3c(ccc4nc(-c5ccccc5)oc43)c21 ; CC1(C)c2cc3ccccc3cc2-c2c(N(c3cccc(N(c4ccccc4)c4ccccc4)c3)c3ccc4oc5ccccc5c4c3)cc3c(ccc4nc(-c5ccccc5)oc43)c21]

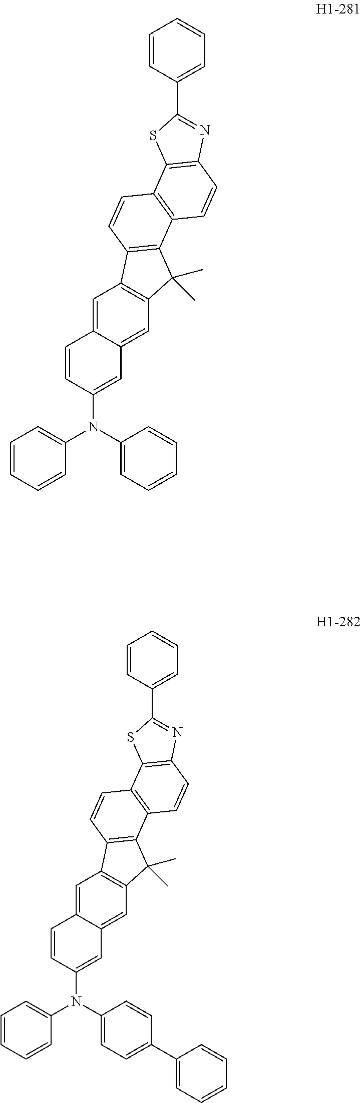 [SMILES: CC1(C)c2cc3cc(N(c4ccccc4)c4ccccc4)ccc3cc2-c2ccc3c(ccc4nc(-c5ccccc5)sc43)c21 ; CC1(C)c2cc3cc(N(c4ccccc4)c4ccc(-c5ccccc5)cc4)ccc3cc2-c2ccc3c(ccc4nc(-c5ccccc5)sc43)c21]

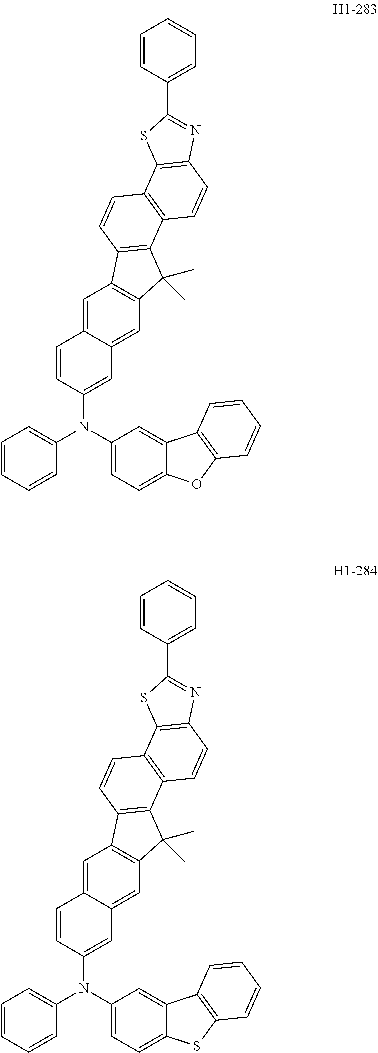 [SMILES: CC1(C)c2cc3cc(N(c4ccccc4)c4ccc5oc6ccccc6c5c4)ccc3cc2-c2ccc3c(ccc4nc(-c5ccccc5)sc43)c21 ; CC1(C)c2cc3cc(N(c4ccccc4)c4ccc5sc6ccccc6c5c4)ccc3cc2-c2ccc3c(ccc4nc(-c5ccccc5)sc43)c21]

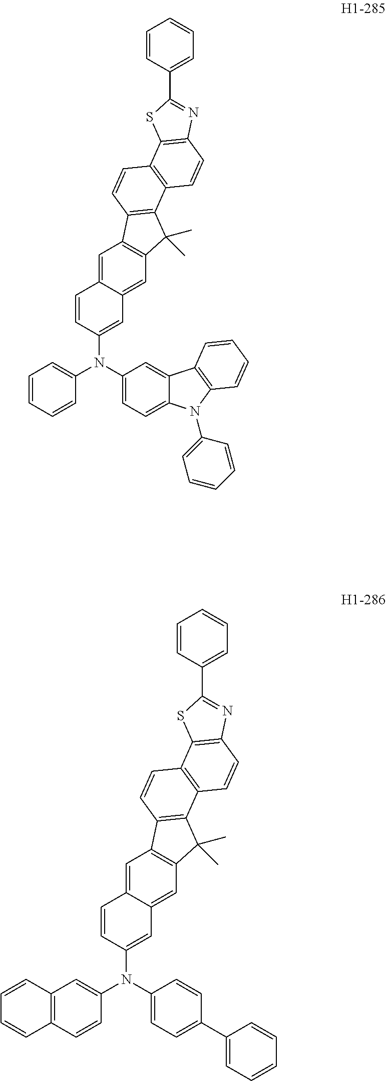 [SMILES: CC1(C)c2cc3cc(N(c4ccccc4)c4ccc5c(c4)c4ccccc4n5-c4ccccc4)ccc3cc2-c2ccc3c(ccc4nc(-c5ccccc5)sc43)c21 ; CC1(C)c2cc3cc(N(c4ccc(-c5ccccc5)cc4)c4ccc5ccccc5c4)ccc3cc2-c2ccc3c(ccc4nc(-c5ccccc5)sc43)c21]

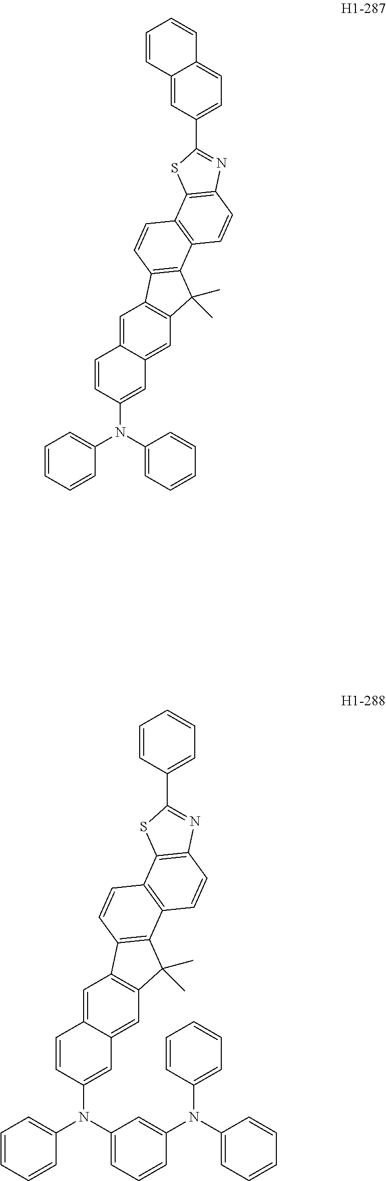 [SMILES: CC1(C)c2cc3cc(N(c4ccccc4)c4ccccc4)ccc3cc2-c2ccc3c(ccc4nc(-c5ccc6ccccc6c5)sc43)c21 ; CC1(C)c2cc3cc(N(c4ccccc4)c4cccc(N(c5ccccc5)c5ccccc5)c4)ccc3cc2-c2ccc3c(ccc4nc(-c5ccccc5)sc43)c21]

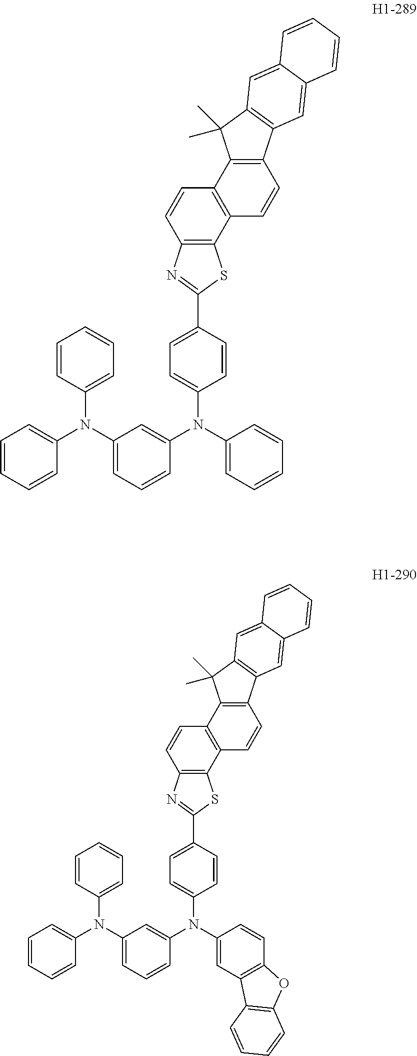 [SMILES: CC1(C)c2cc3ccccc3cc2-c2ccc3c(ccc4nc(-c5ccc(N(c6ccccc6)c6cccc(N(c7ccccc7)c7ccccc7)c6)cc5)sc43)c21 ; CC1(C)c2cc3ccccc3cc2-c2ccc3c(ccc4nc(-c5ccc(N(c6cccc(N(c7ccccc7)c7ccccc7)c6)c6ccc7oc8ccccc8c7c6)cc5)sc43)c21]

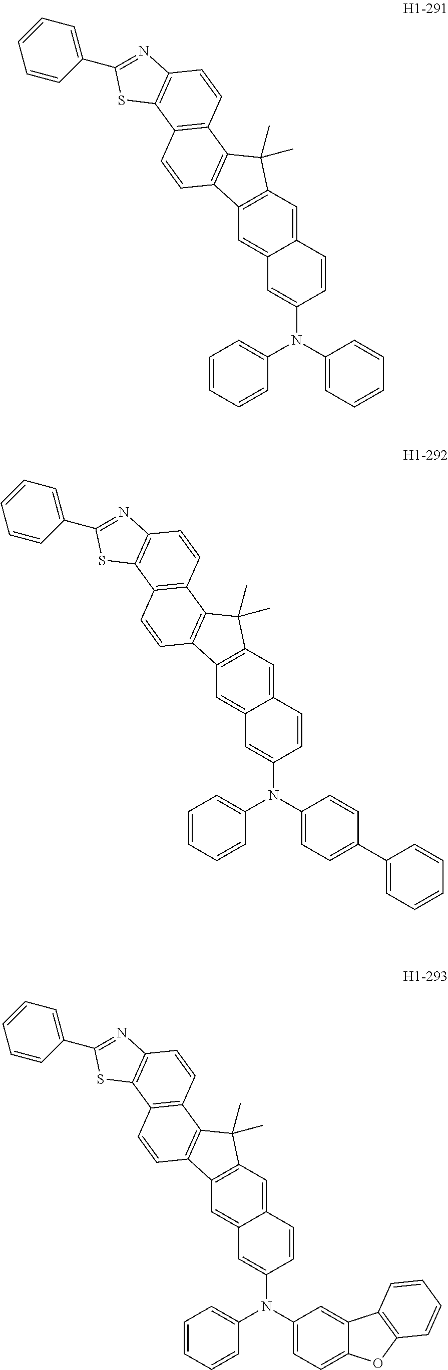 [SMILES: CC1(C)c2cc3ccc(N(c4ccccc4)c4ccccc4)cc3cc2-c2ccc3c(ccc4nc(-c5ccccc5)sc43)c21 ; CC1(C)c2cc3ccc(N(c4ccccc4)c4ccc(-c5ccccc5)cc4)cc3cc2-c2ccc3c(ccc4nc(-c5ccccc5)sc43)c21 ; CC1(C)c2cc3ccc(N(c4ccccc4)c4ccc5oc6ccccc6c5c4)cc3cc2-c2ccc3c(ccc4nc(-c5ccccc5)sc43)c21]

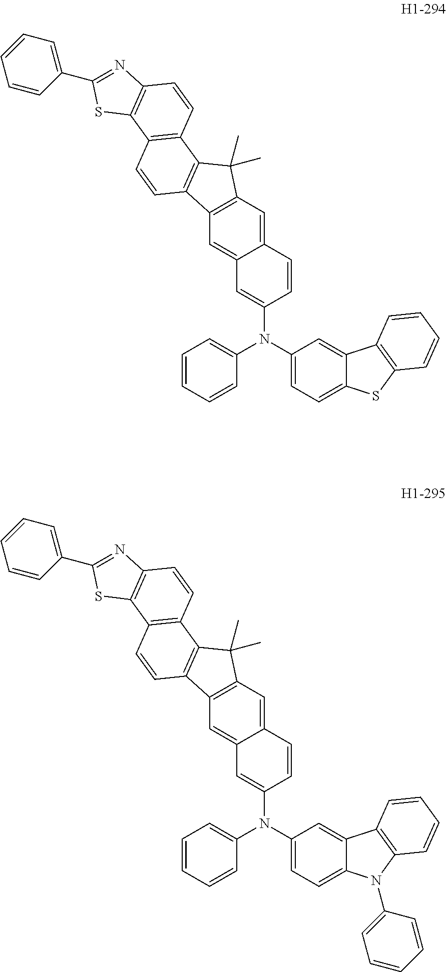 [SMILES: CC1(C)c2cc3ccc(N(c4ccccc4)c4ccc5sc6ccccc6c5c4)cc3cc2-c2ccc3c(ccc4nc(-c5ccccc5)sc43)c21 ; CC1(C)c2cc3ccc(N(c4ccccc4)c4ccc5c(c4)c4ccccc4n5-c4ccccc4)cc3cc2-c2ccc3c(ccc4nc(-c5ccccc5)sc43)c21]

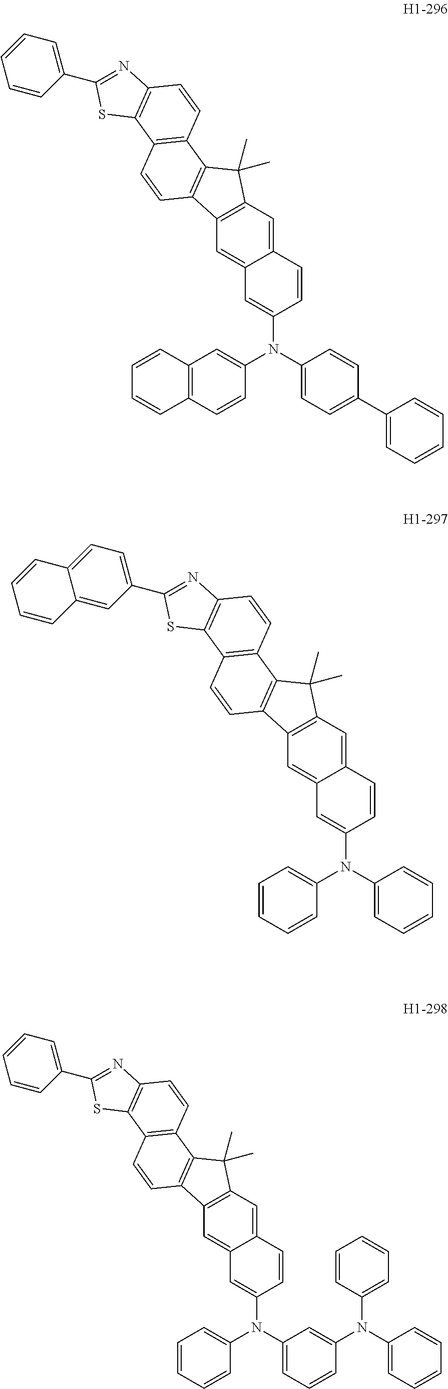 [SMILES: CC1(C)c2cc3ccc(N(c4ccc(-c5ccccc5)cc4)c4ccc5ccccc5c4)cc3cc2-c2ccc3c(ccc4nc(-c5ccccc5)sc43)c21 ; CC1(C)c2cc3ccc(N(c4ccccc4)c4ccccc4)cc3cc2-c2ccc3c(ccc4nc(-c5ccc6ccccc6c5)sc43)c21 ; CC1(C)c2cc3ccc(N(c4ccccc4)c4cccc(N(c5ccccc5)c5ccccc5)c4)cc3cc2-c2ccc3c(ccc4nc(-c5ccccc5)sc43)c21]

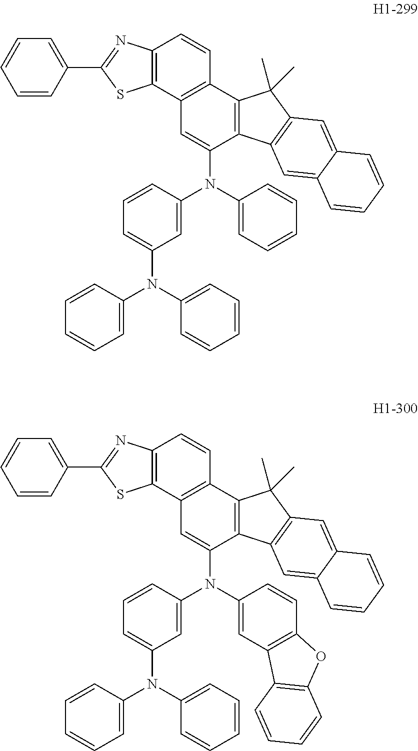 [SMILES: CC1(C)c2cc3ccccc3cc2-c2c(N(c3ccccc3)c3cccc(N(c4ccccc4)c4ccccc4)c3)cc3c(ccc4nc(-c5ccccc5)sc43)c21 ; CC1(C)c2cc3ccccc3cc2-c2c(N(c3cccc(N(c4ccccc4)c4ccccc4)c3)c3ccc4oc5ccccc5c4c3)cc3c(ccc4nc(-c5ccccc5)sc43)c21]

The organic electroluminescent compound of the present disclosure of the formula 1′ may be comprised in at least one layer of a light-emitting layer, a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer, and preferably, may be comprised in at least one layer of a light-emitting layer, a hole transport layer, a hole auxiliary layer, a light-emitting auxiliary layer, an electron transport layer, an electron buffer layer, a hole blocking layer, and an electron blocking layer. When used in a light-emitting layer, the organic electroluminescent compound of the present disclosure of the formula 1′ may be comprised as a host material, and may also be comprised as a hole transport layer, a hole auxiliary layer, an electron blocking layer, and a hole transport zone. If necessary, the organic electroluminescent compound of the present disclosure can be used as a co-host material.

The compound represented by formula 1, 2, and 1′ according to the present disclosure may be produced by referring to the following Reaction Schemes, but is not limited thereto.

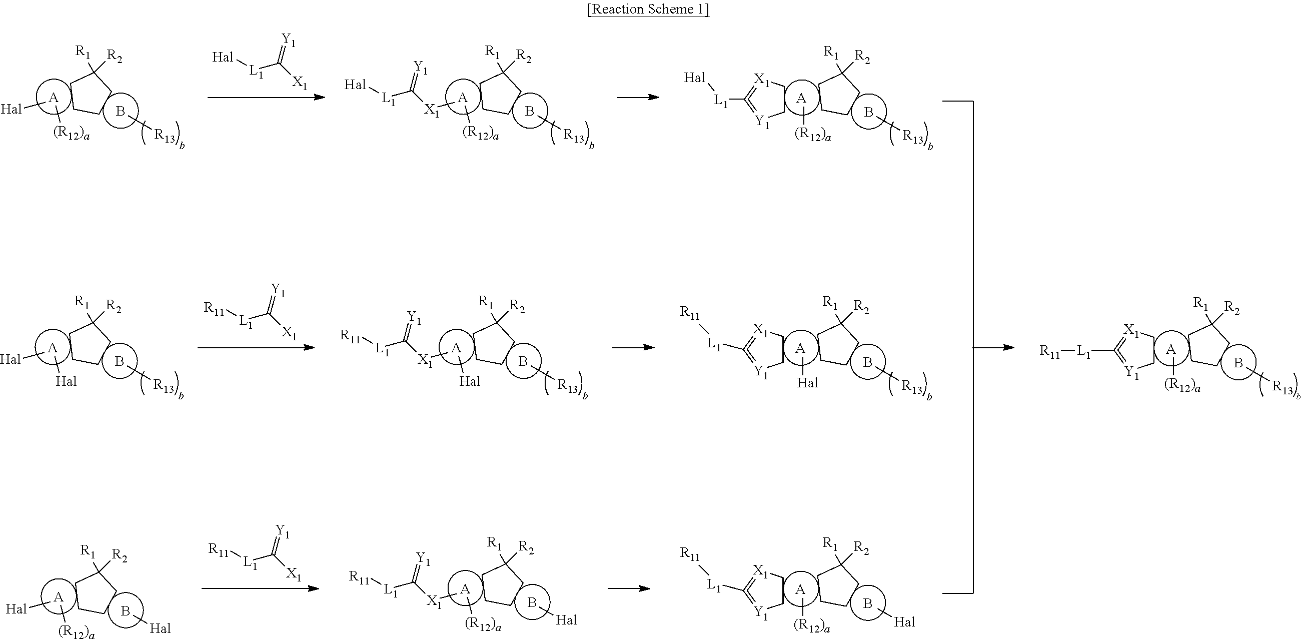

In Reaction Scheme 1, R₁, R₂, R₁₁ to R₁₃, X₁, Y₁, L₁, a and b are as defined in formula 1 and formula 1′, and Hal refers to a halogen.

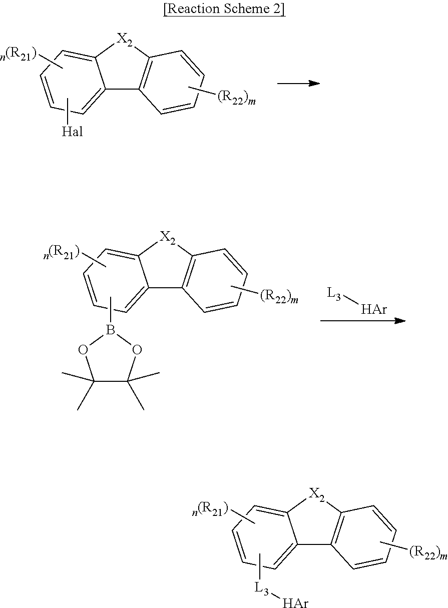

In Reaction Scheme 2, R₂₁, R₂₂, X₂, L₃, HAr, n and m are as defined in formula 2.

Although illustrative synthesis examples of the compound represented by formulas 1, 2, and 1 are described above, one skilled in the art will be able to readily understand that they are all based on a Buchwald-Hartwig cross coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, and a Wittig reaction, etc., and the above reactions proceed even when substituents defined in formulas 1, 2, and 1′ other than the substituents specified in the specific synthesis examples are bonded.

The present disclosure provides an organic electroluminescent device comprising an anode; a cathode; and at least one light-emitting layer between the anode and the cathode, wherein the at least one light-emitting layer comprises the plurality of host materials according to the present disclosure. The first host material and the second host material of the present disclosure may be comprised in one light-emitting layer or may be respectively comprised in different light-emitting layers among a plurality of light-emitting layers. The plurality of host materials of the present disclosure may comprise the compound represented by formula 1 and the compound represented by formula 2 at a ratio of about 1:99 to about 99:1, preferably about 10:90 to about 90:10, more preferably about 30:70 to about 70:30, further more preferably about 50:50. In addition, the compound represented by formula 1 and the compound represented by formula 2 in a desired ratio may be combined by mixing them in a shaker, by dissolving them in a glass tube by heat, or by dissolving them in a solvent, etc.

According to one embodiment of the present disclosure, the doping concentration of the dopant compound with respect to the host compound of the light-emitting layer may be less than 20 wt %. The dopants comprised in the organic electroluminescent device of the present disclosure may be at least one phosphorescent or fluorescent dopant, and is preferably a phosphorescent dopant. The phosphorescent dopant materials applied to the organic electroluminescent device according to the present disclosure are not particularly limited, but may be selected from metallated complex compounds of iridium (Ir), osmium (Os), copper (Cu), and platinum (Pt), preferably selected from ortho-metallated complex compounds of iridium (Ir), osmium (Os), copper (Cu), and platinum (Pt), and more preferably an ortho-metallated iridium complex compound.

The dopant comprised in the organic electroluminescent device of the present disclosure may be a compound represented by the following formula 101, but is not limited thereto.

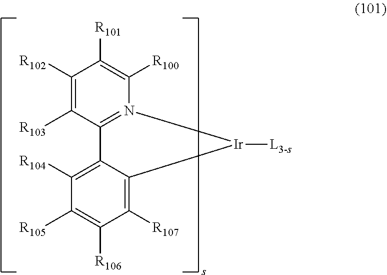

In formula 101,
L is any one selected from the following structures 1 to 3;

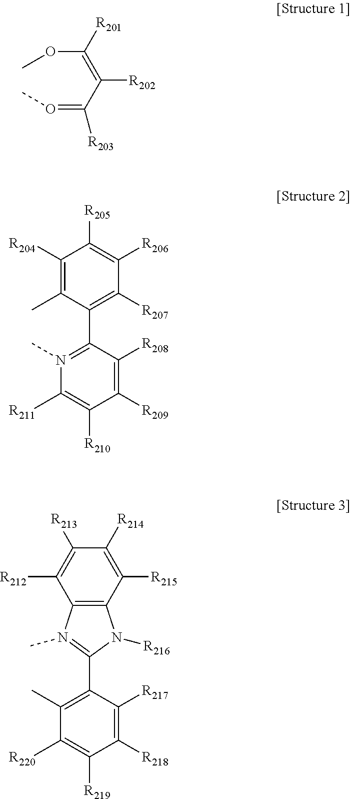

R₁₀₀ to R₁₀₃, each independently, represent hydrogen, deuterium, a halogen, a (C1—C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and
s represents an integer of 1 to 3.

The specific examples of the dopant compound are as follows, but are not limited thereto.

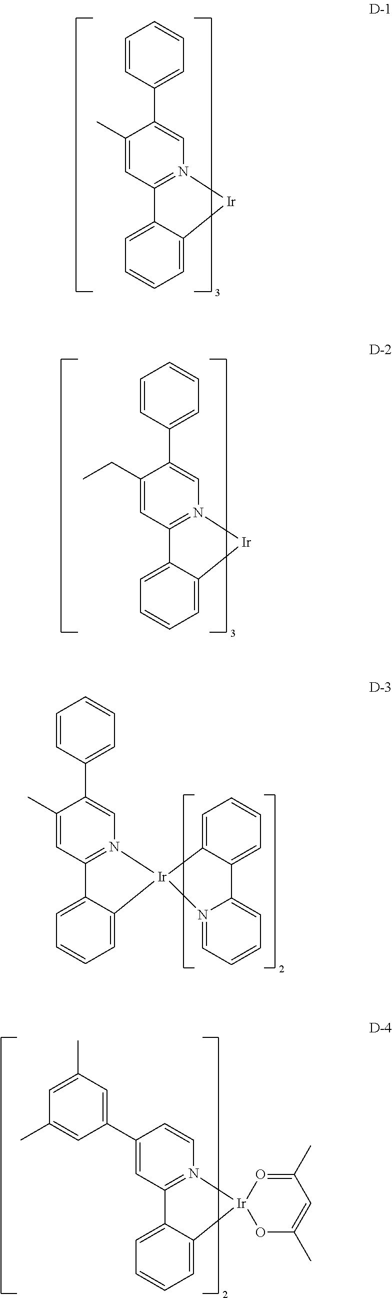

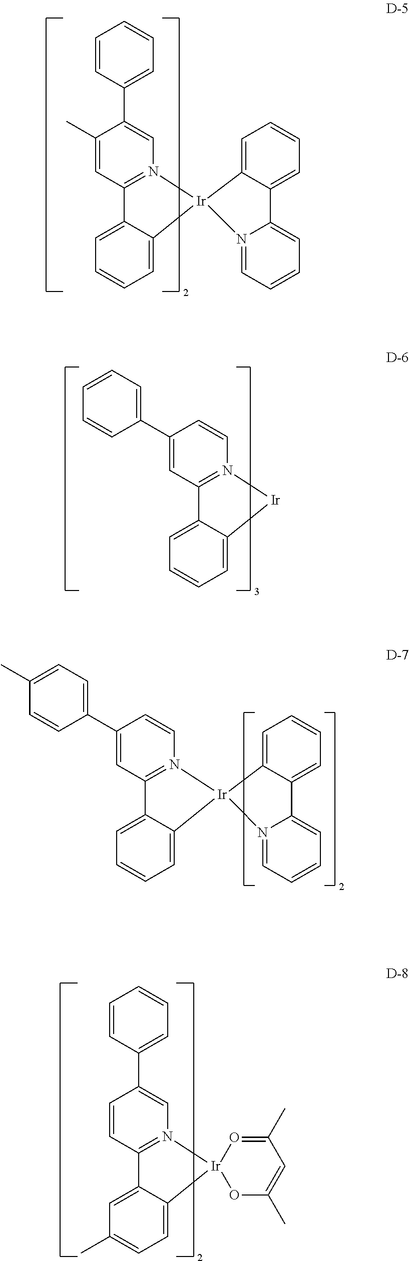

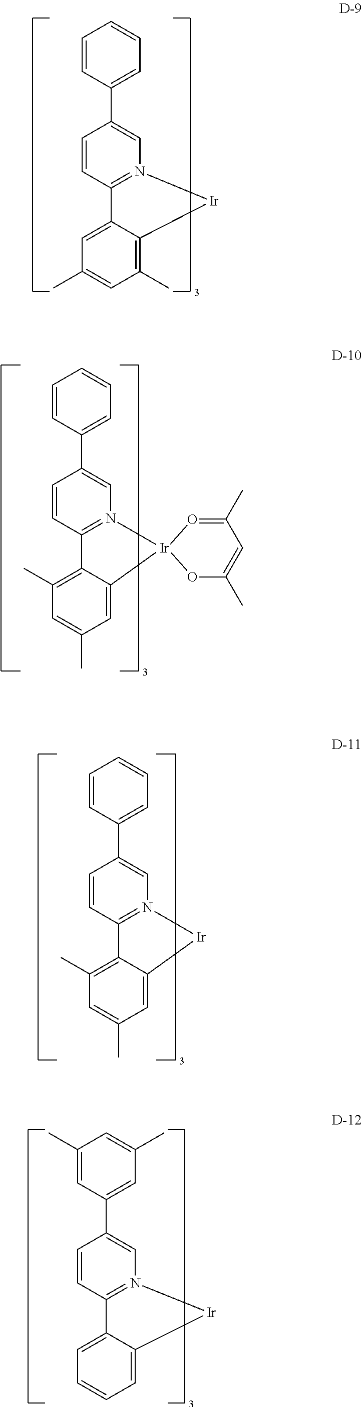

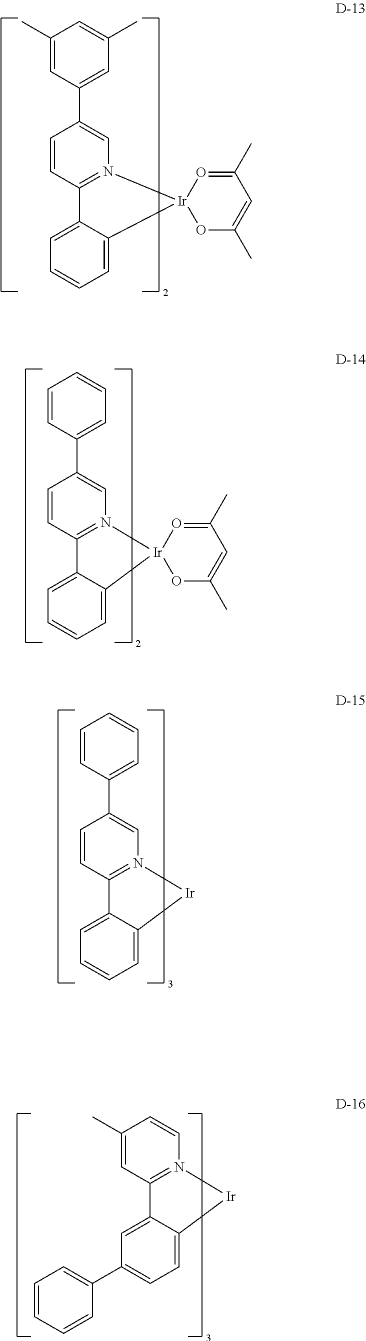

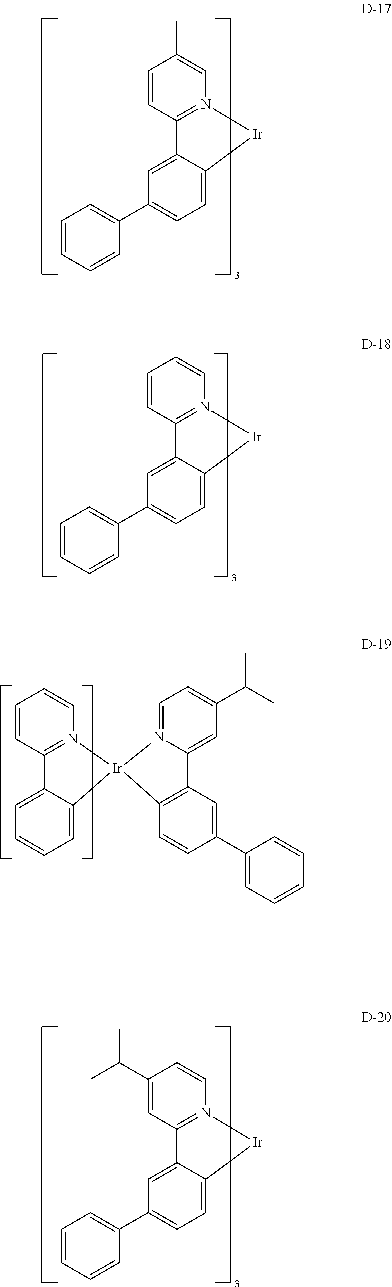

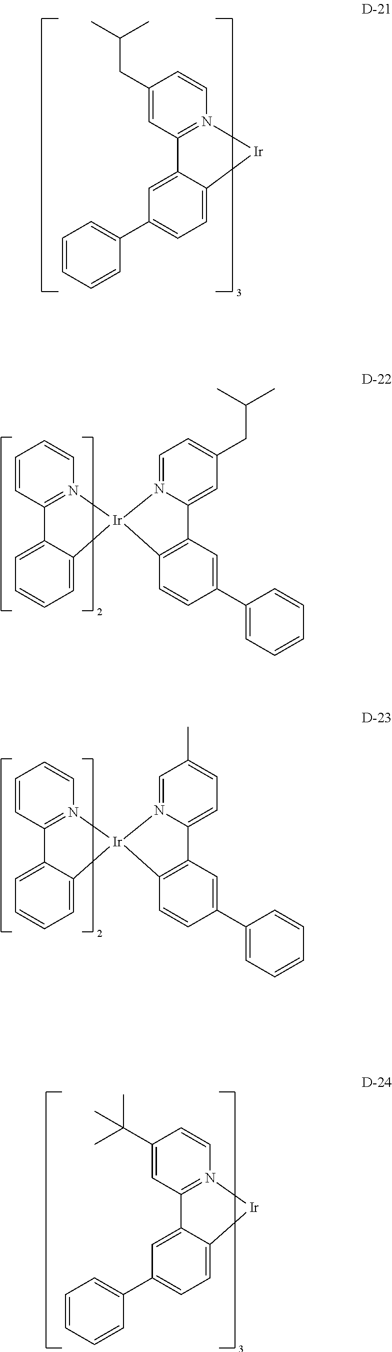

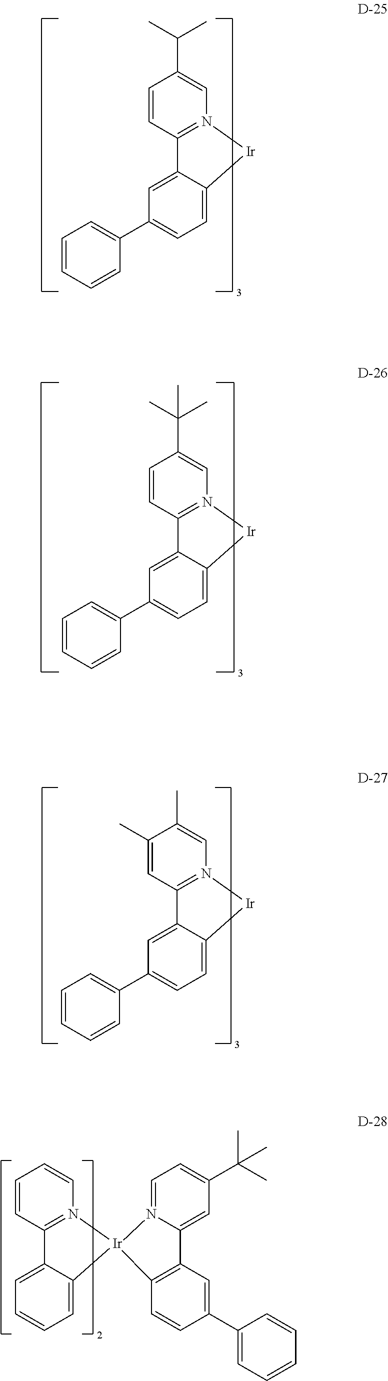

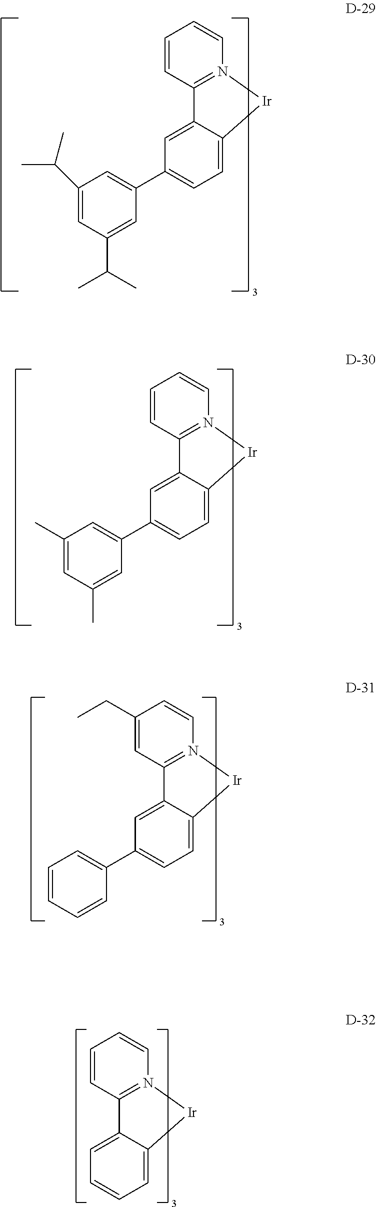

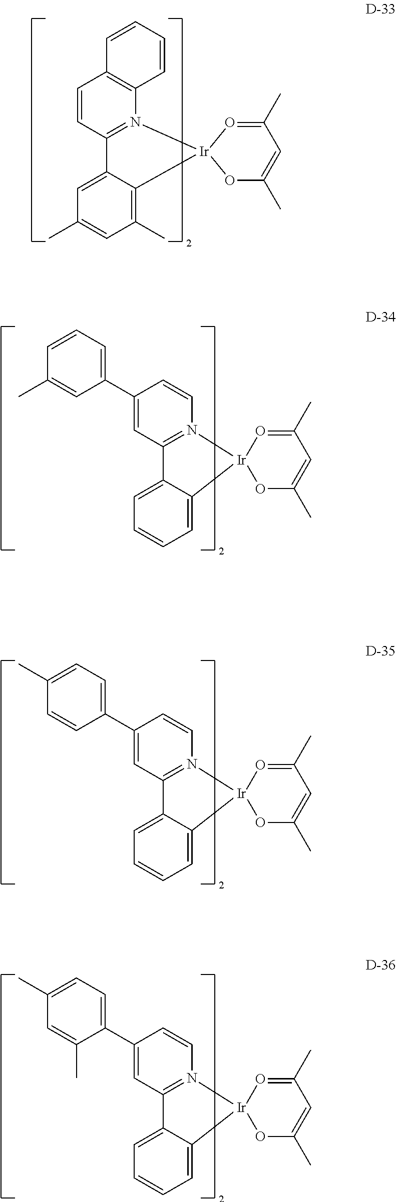

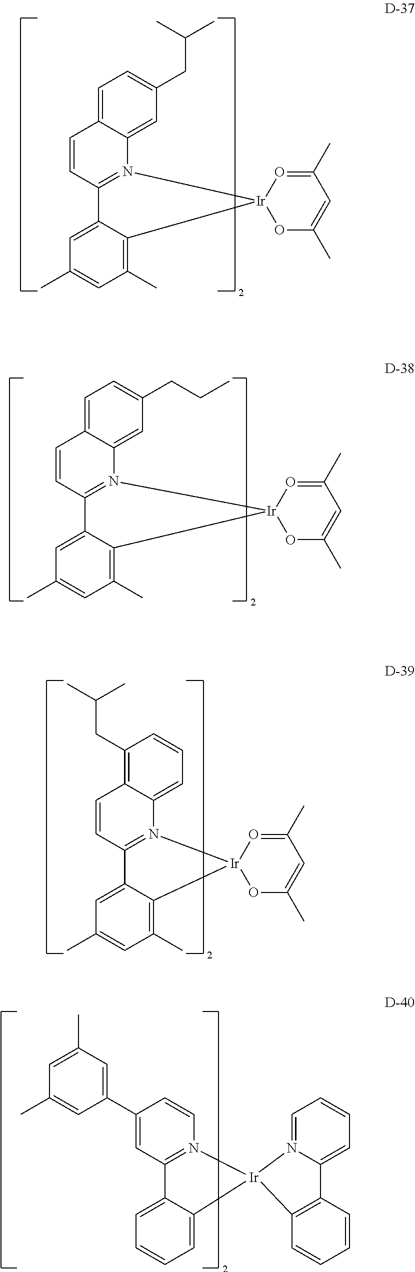

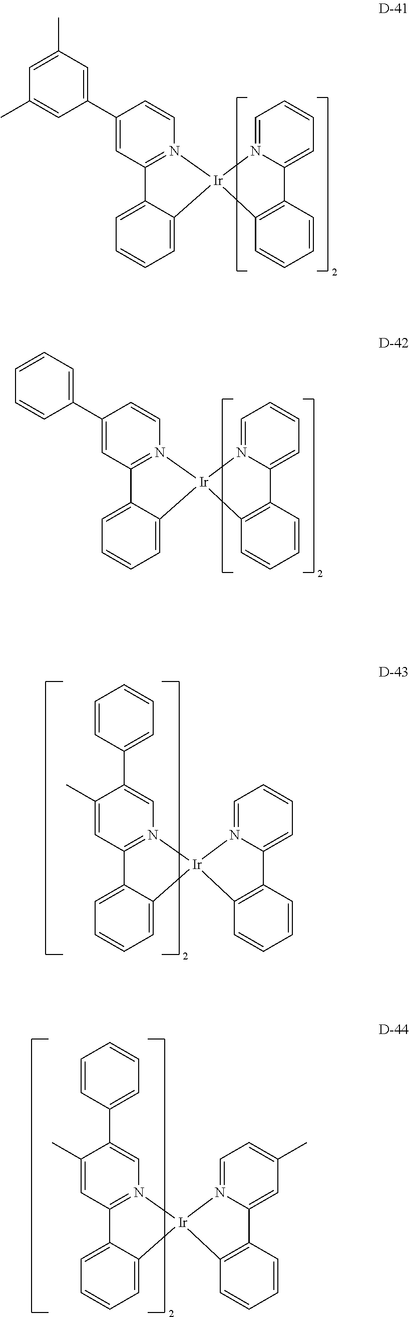

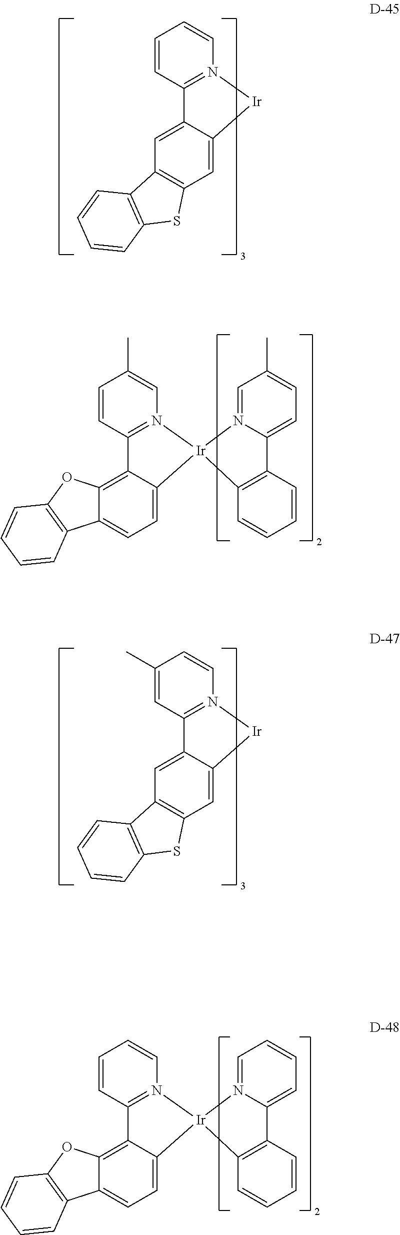

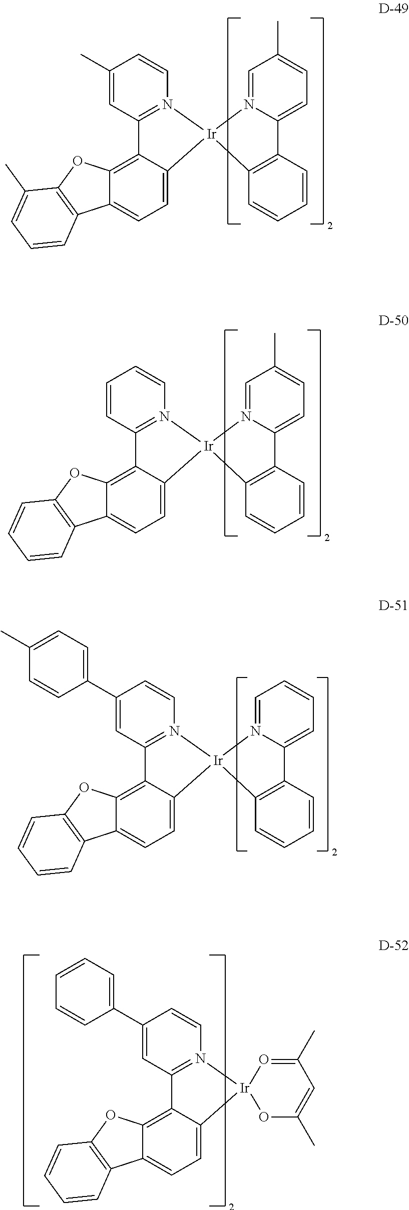

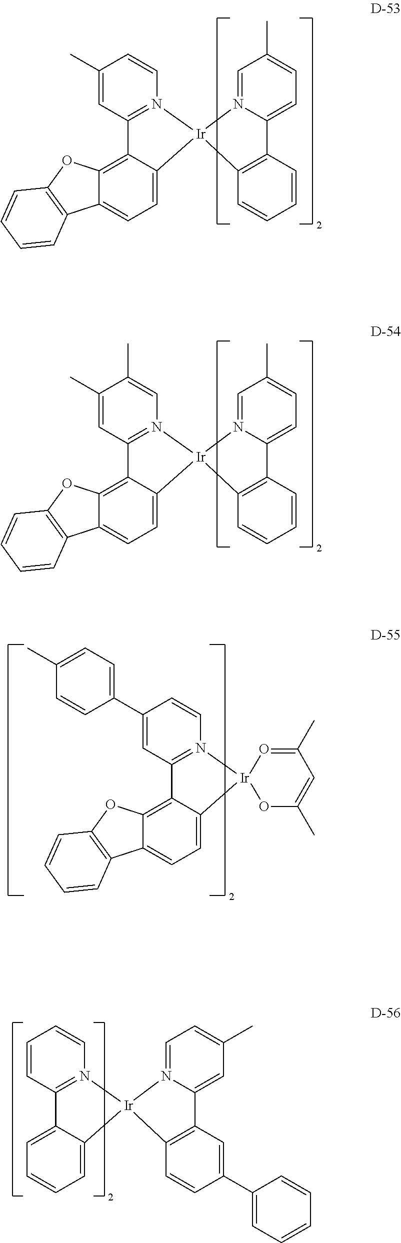

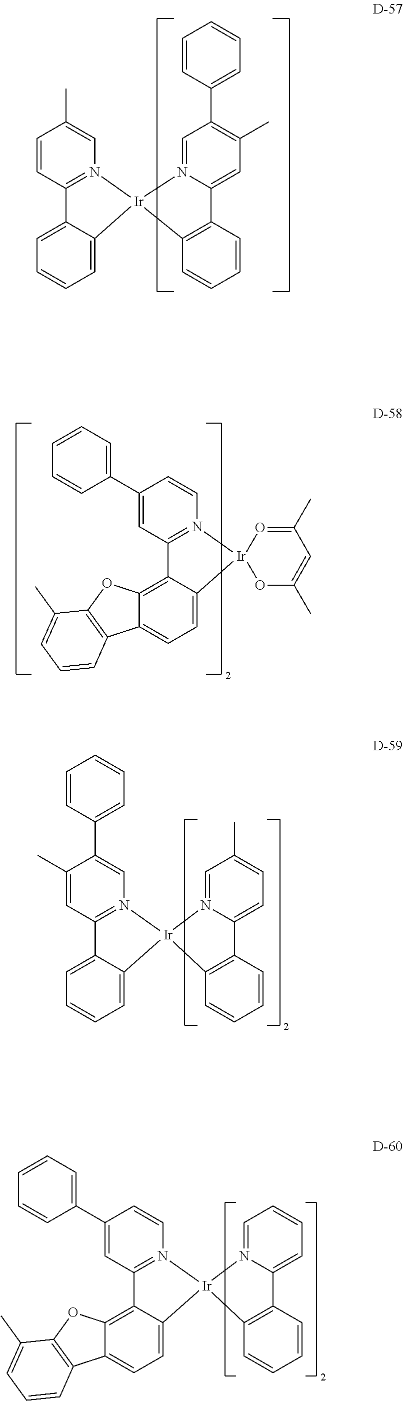

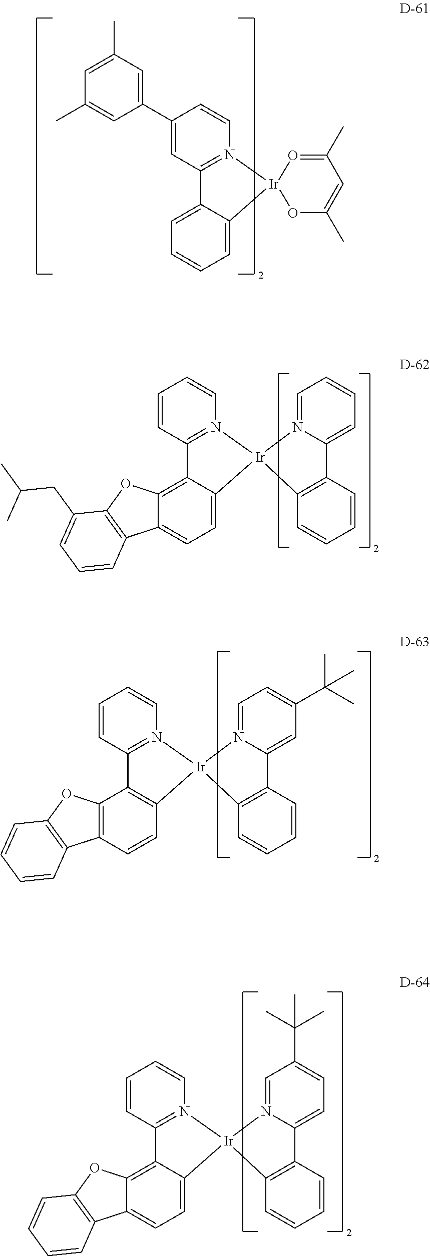

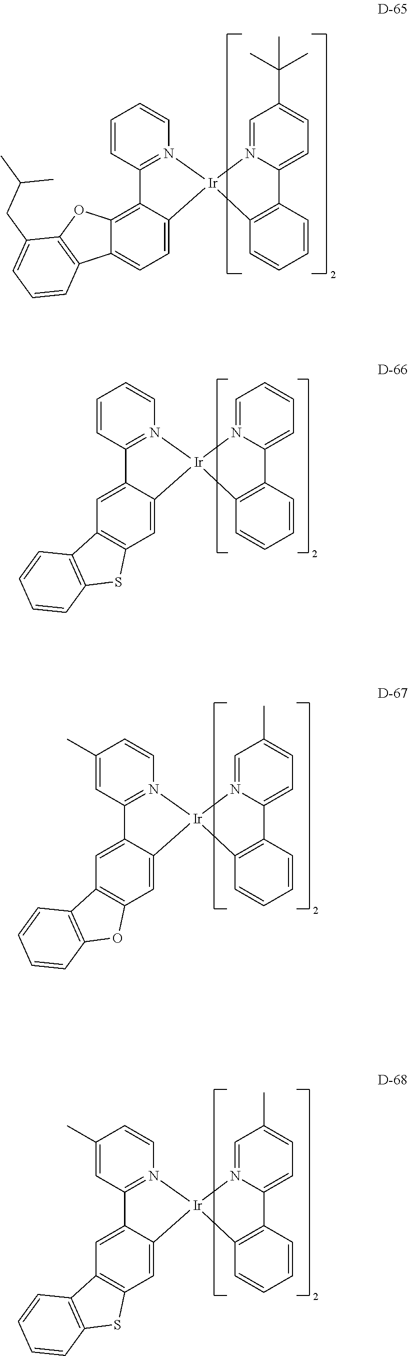

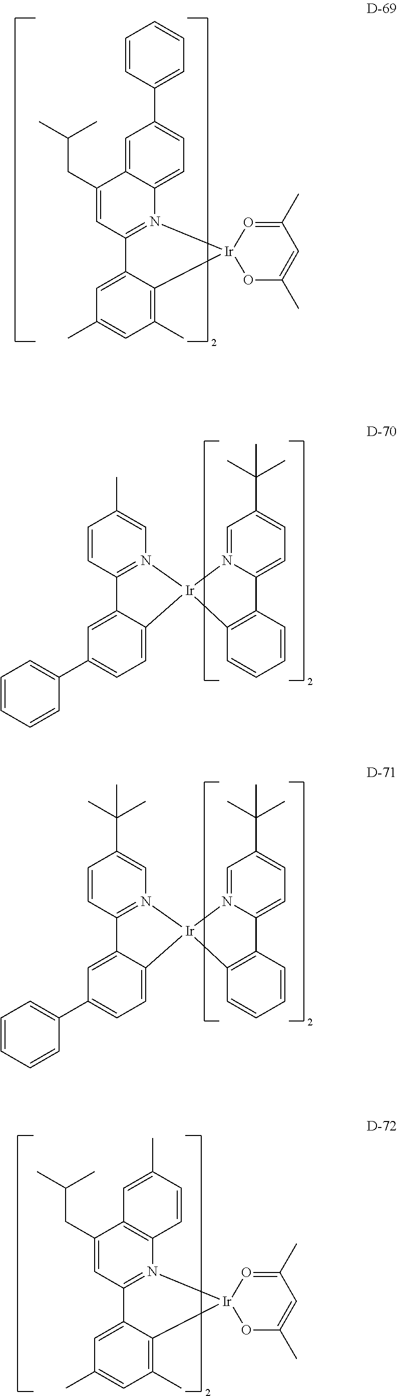

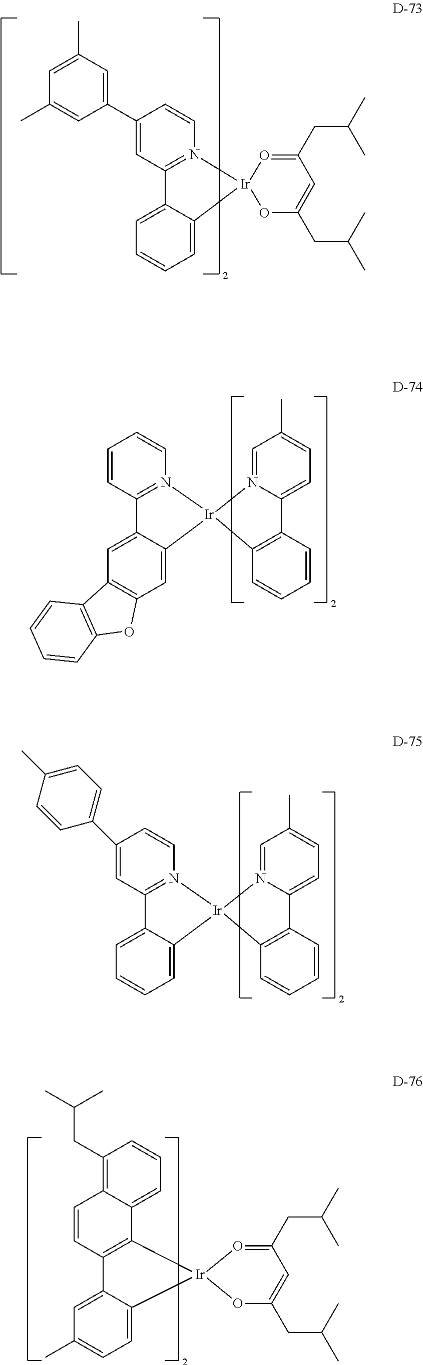

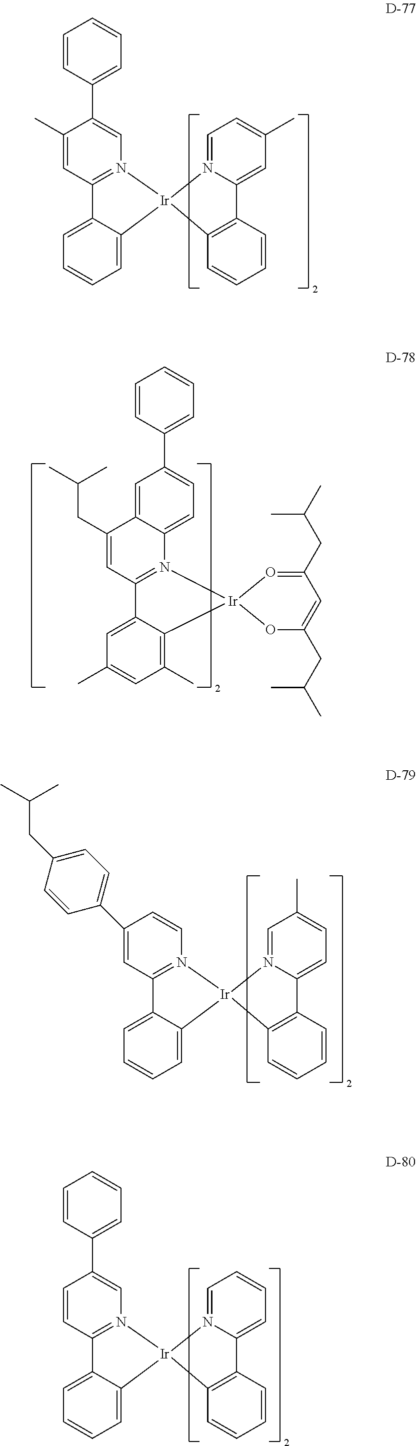

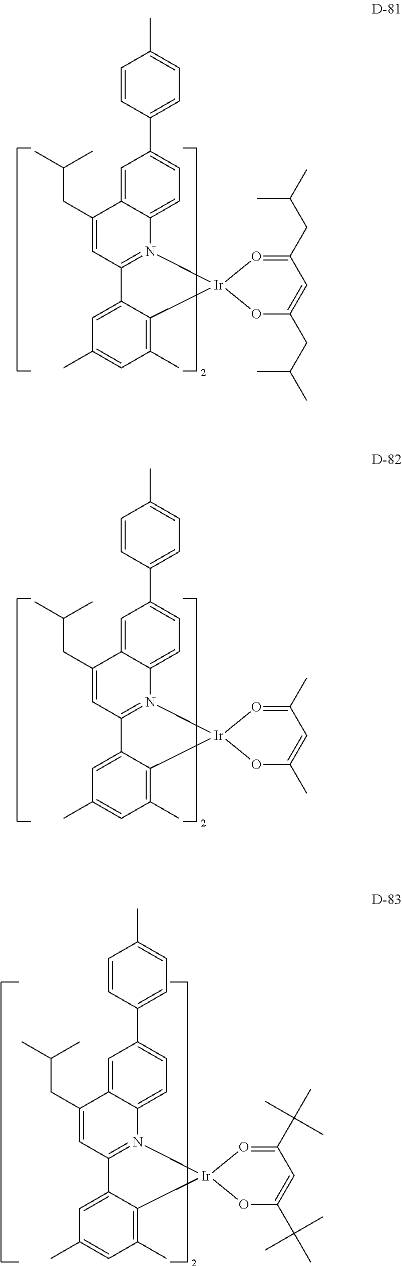

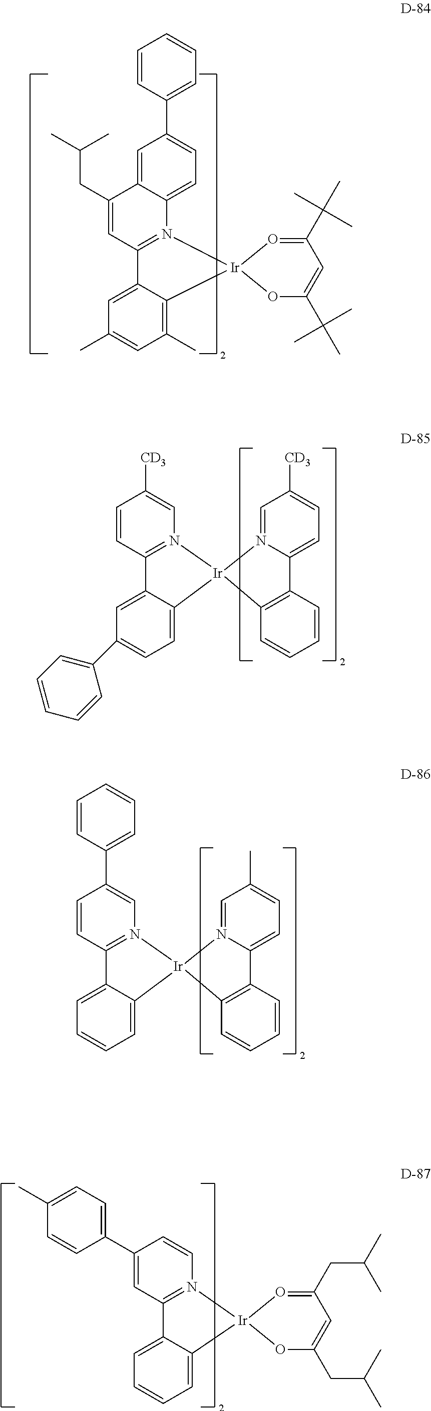

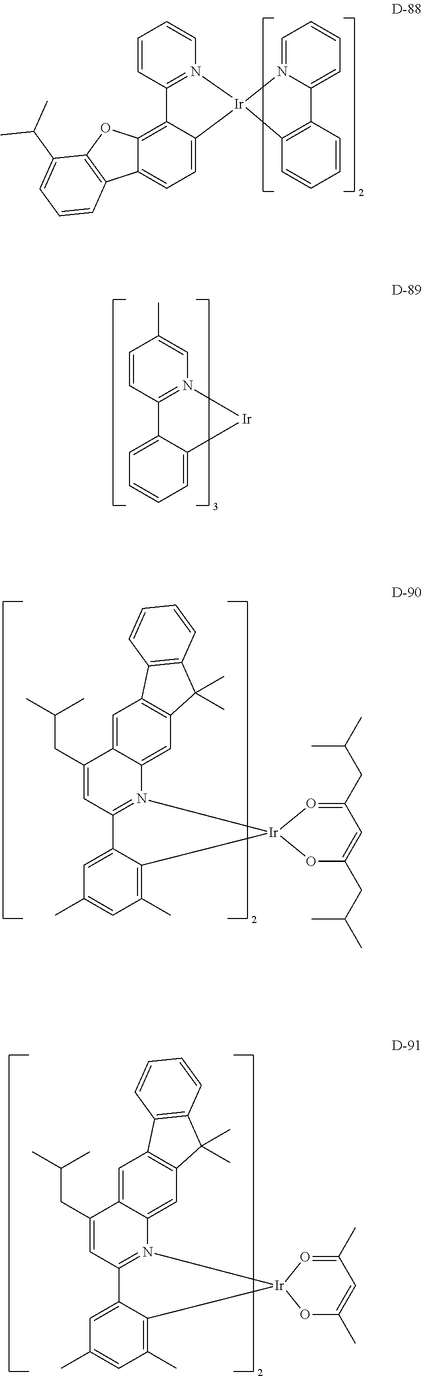

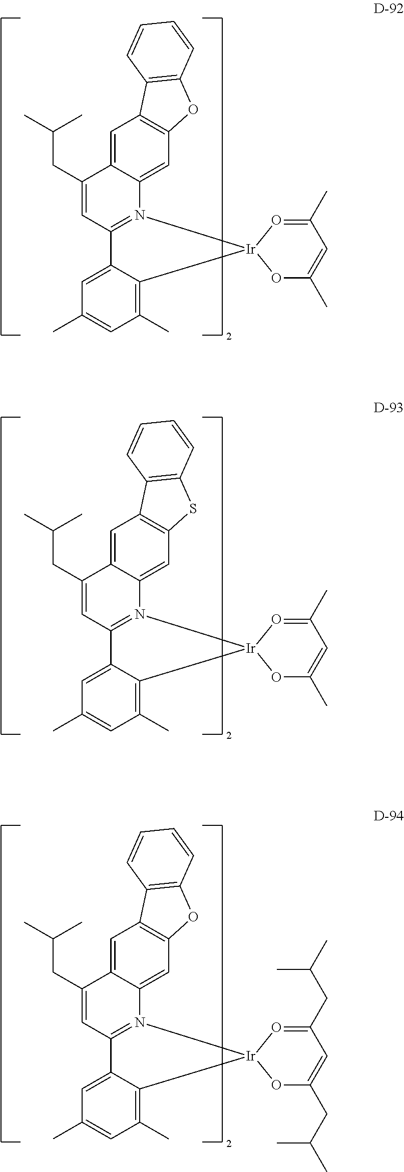

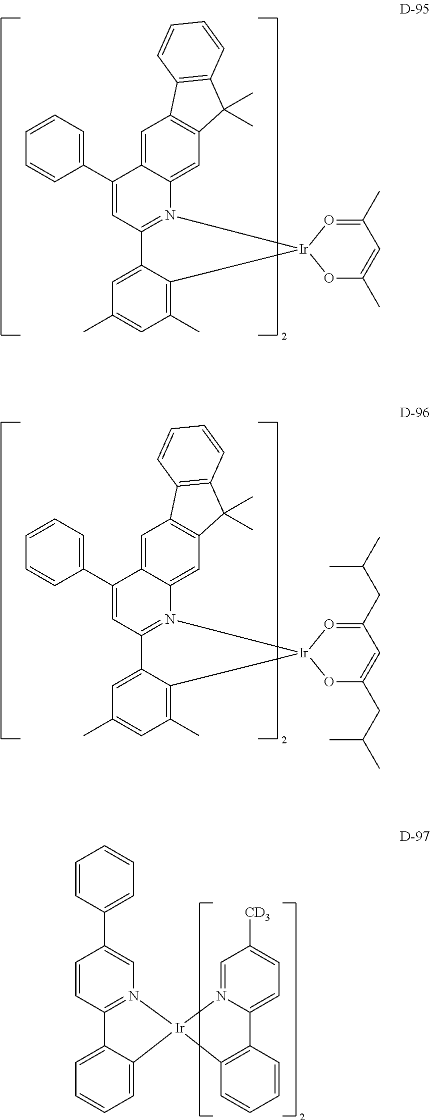

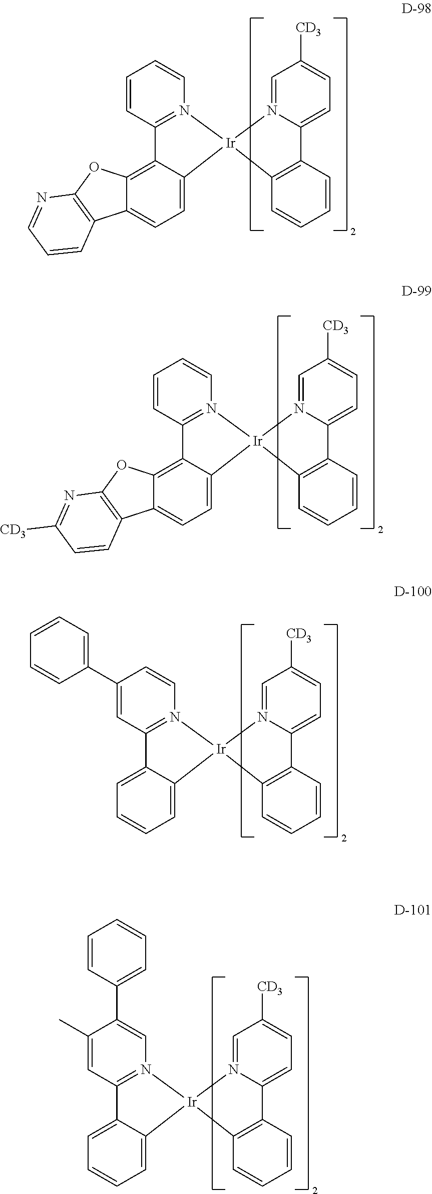

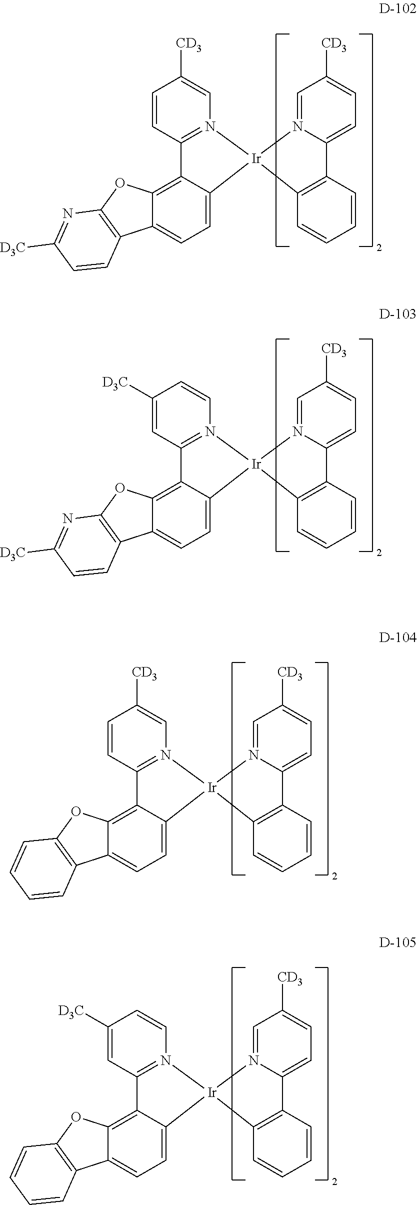

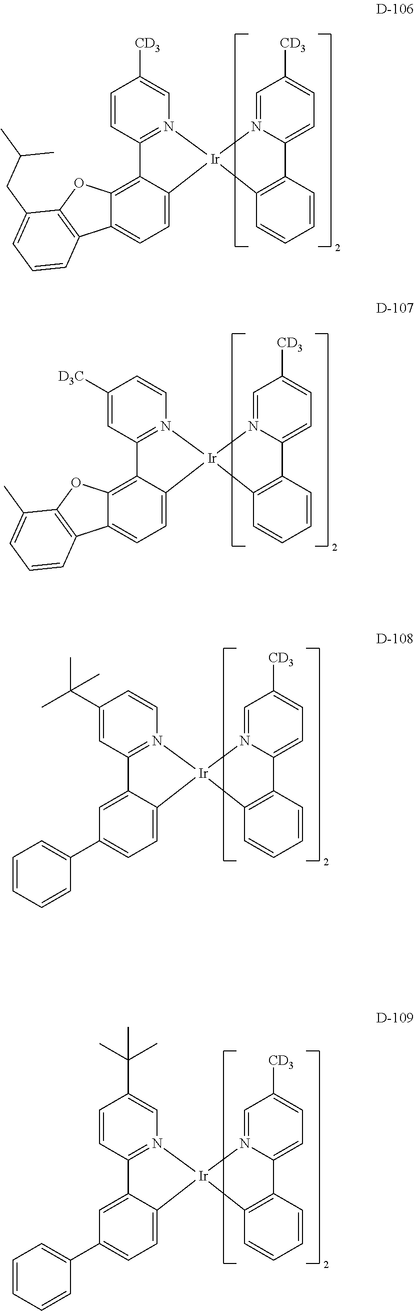

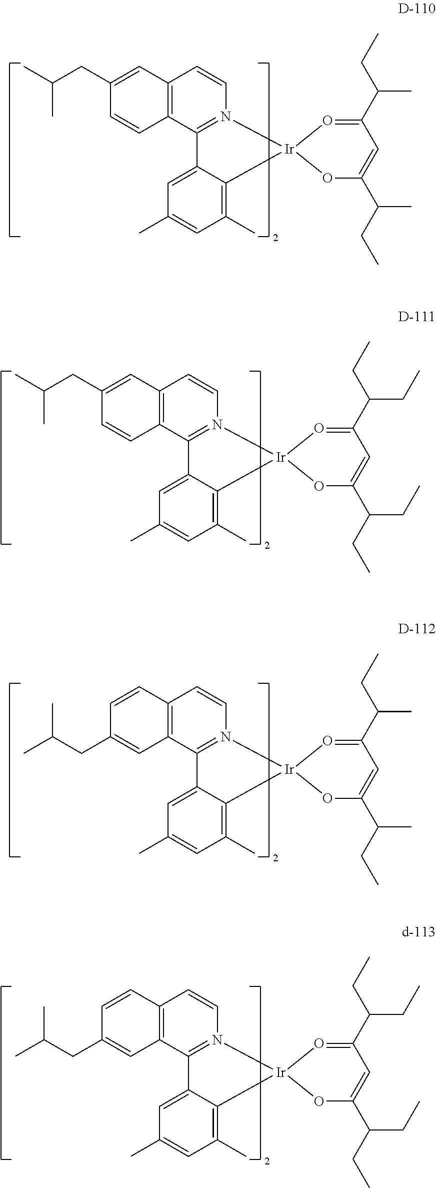

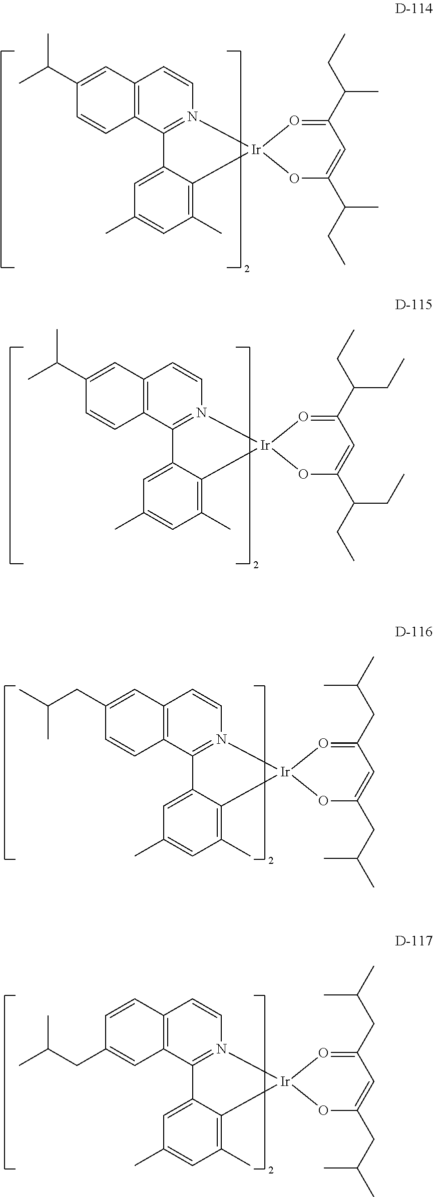

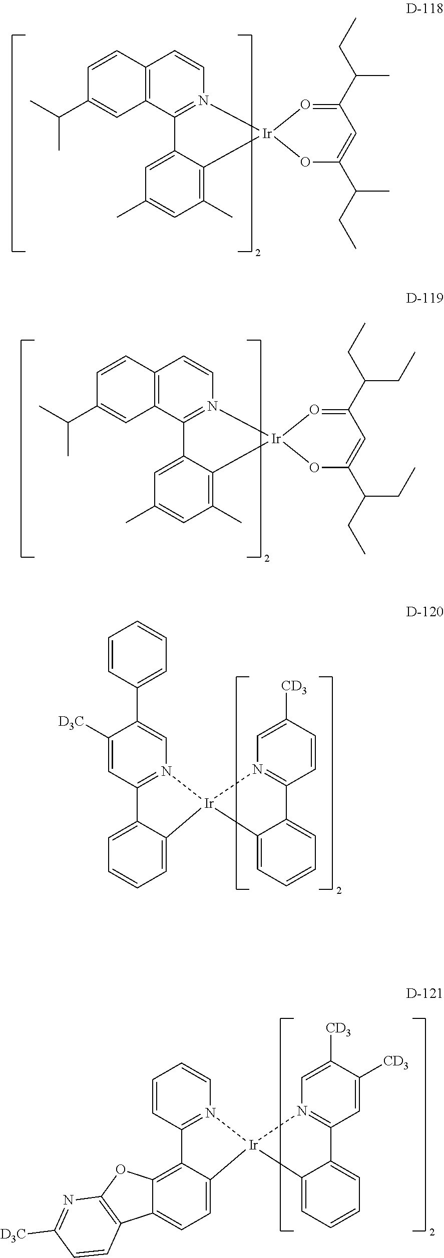

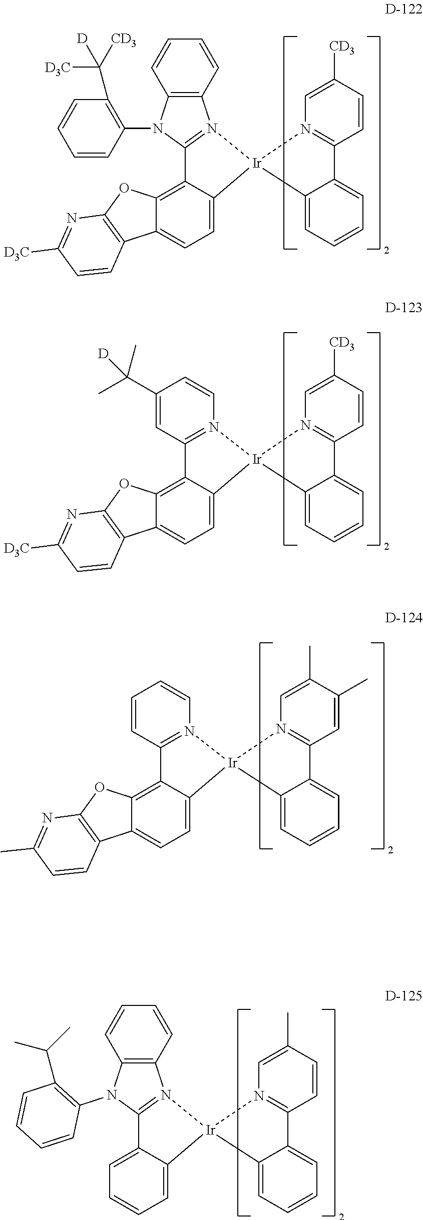

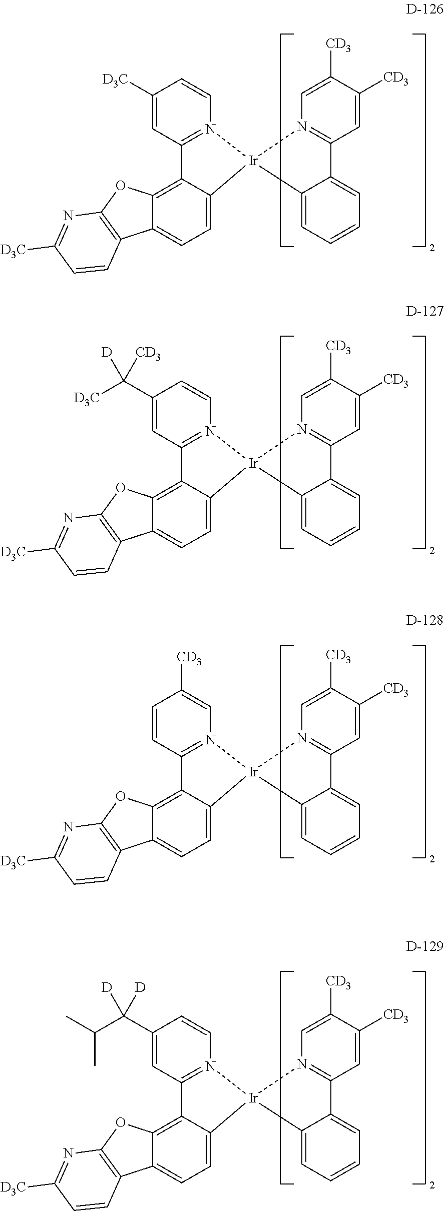

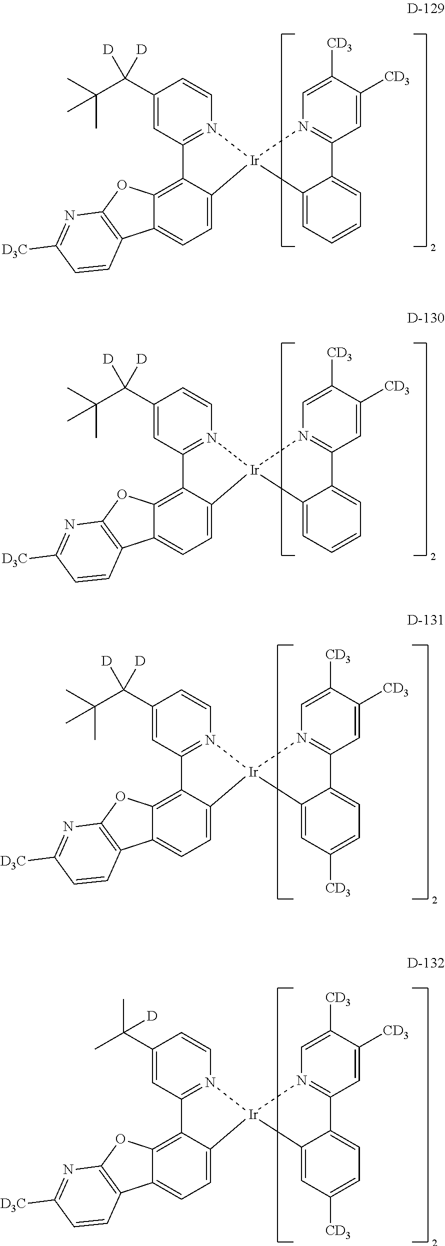

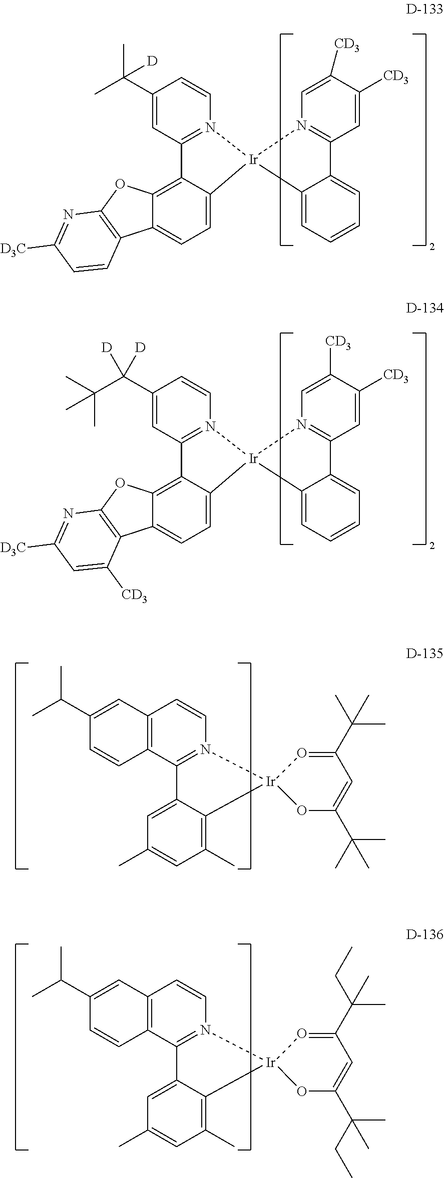

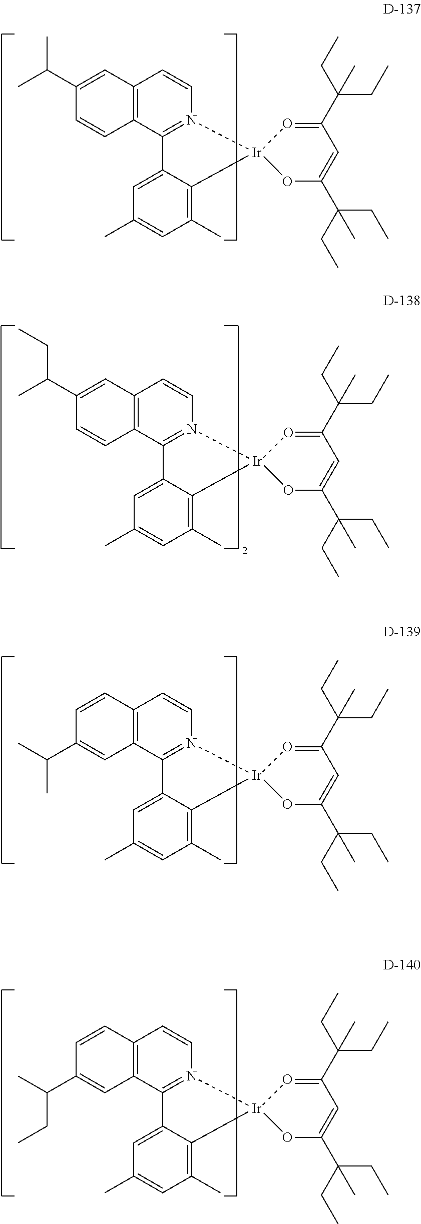

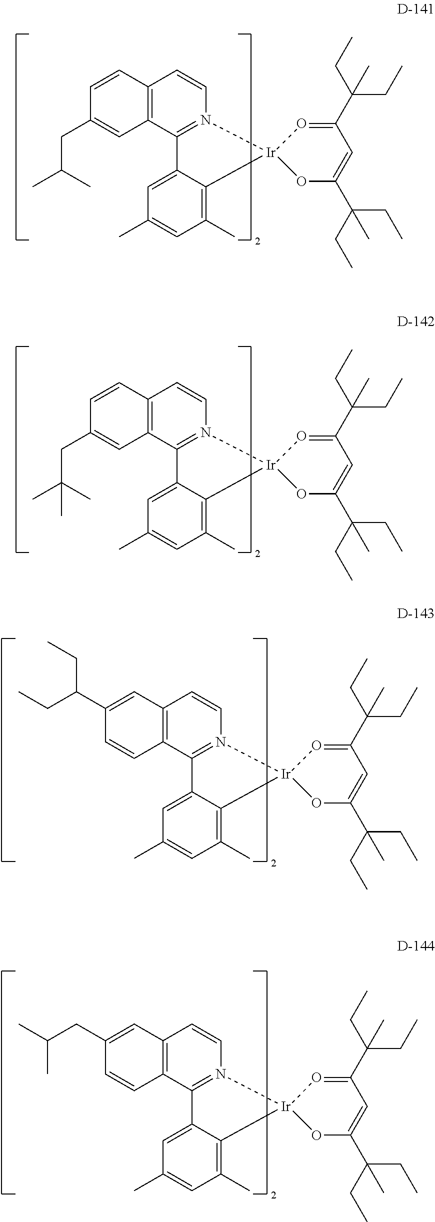

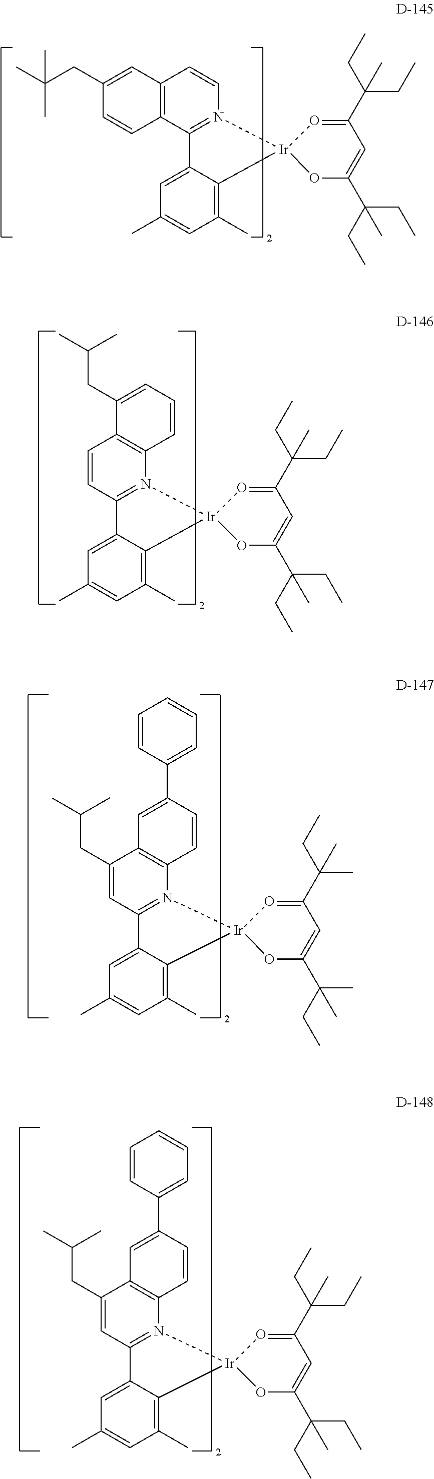

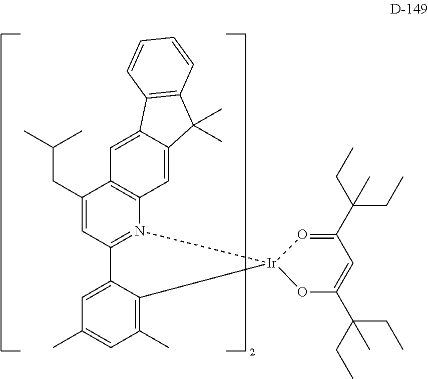

The organic electroluminescent device of the present disclosure comprises an anode; a cathode; and at least one organic layer between the anode and the cathode. The organic layer comprises a light-emitting layer, and may further comprise at least one layer selected from any of the following: a hole injection layer, a hole transport layer, a hole auxiliary layer, an light-emitting auxiliary layer, an electron transport layer, an electron buffer layer, an electron injection layer, an interlayer, a hole blocking layer, and an electron blocking layer. Each of the layers may be further configured as a plurality of layers.

Each of the anode and the cathode may be formed of a transparent conductive material or a transflective or reflective conductive material. Depending on the type of material forming the anode and the cathode, the organic electroluminescent device may be a top light-emitting type, a bottom light-emitting type, or a double side light-emitting type. In addition, the hole injection layer may be further doped with a p-dopant(s), and the electron injection layer may be further doped with an n-dopant(s).

At least one compound(s) selected from the group consisting of arylamine-based compounds and styrylarylamine-based compounds may be further comprised in the organic layer. In addition, the organic material layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising the metal thereof.

In addition, the organic electroluminescent device of the present disclosure may emit white light by further comprising at least one light-emitting layer, which comprises a blue, a red, or a green light-emitting compound known in the art, besides the compound of the present disclosure. In addition, if necessary, it may further comprise a yellow or an orange light-emitting layer.

In the organic electroluminescent device of the present disclosure, it is preferable to dispose at least one layer selected from a chalcogenide layer, a metal halide layer, and a metal oxide layer (hereinafter referred to as “surface layer”) on at least one inner surface of a pair of electrodes. Specifically, a chalcogenide (including oxide) layer of silicon and aluminum is preferably placed on an anode surface of an electroluminescent medium layer side, and a metal halide layer or a metal oxide layer is preferably placed on a cathode surface of an electroluminescent medium layer side. Driving stabilization of the organic electroluminescent device can be obtained by the surface layer. Preferred examples of the chalcogenide include SiOₓ(1≤X≤2), AlOₓ(1≤X≤1.5), SiON, SiAlON, etc., preferred examples of the metal halide include LiF, MgF₂, CaF₂, a rare earth metal fluoride, etc, and preferred examples of the metal oxide include Cs₂O, Li₂O, MgO, SrO, BaO, CaO, etc.

A hole injection layer, a hole transport layer or an electron blocking layer, or a combination thereof may be used between an anode and a light-emitting layer. The hole injection layer may be multi-layered in order to lower the hole injection barrier (or hole injection voltage) from the anode to the hole transport layer or the electron blocking layer, wherein two compounds may be simultaneously used in each of the multi-layers. The hole transport layer or the electron blocking layer may be multi-layered.

An electron buffer layer, a hole blocking layer, an electron transport layer or an electron injection layer, or a combination thereof may be used between a light-emitting layer and a cathode. The electron buffer layer may be multi-layered in order to control electron injection and improve interfacial properties between the light-emitting layer and the electron injection layer, wherein two compounds may be simultaneously used in each of the multi-layers. The hole blocking layer or the electron transport layer may be multi-layered, wherein a plurality of compounds may be used in each of the multi-layers.

A light-emitting auxiliary layer may be a layer placed between an anode and a light-emitting layer, or between a cathode and a light-emitting layer. When placed between the anode and the light-emitting layer, the light-emitting auxiliary layer may be used to facilitate hole injection and/or hole transport or to block the overflow of electrons. When placed between the cathode and the light-emitting layer, the light-emitting auxiliary layer may be used to facilitate electron injection and/or electron transport or to block the overflow of holes. In addition, the hole auxiliary layer may be placed between the hole transport layer (or hole injection layer) and the light-emitting layer, and may exhibit an effect of facilitating or blocking the hole transport rate (or hole injection rate), and accordingly, may adjust the charge balance. In addition, the electron blocking layer may be placed between the hole transport layer (or hole injection layer) and the light-emitting layer, and may block the overflow of electrons from the light-emitting layer and confine the excitons in the light-emitting layer to prevent light leakage. When an organic electroluminescent device includes two or more hole transport layers, the hole transport layer, which is further included, may be used as a hole auxiliary layer or an electron blocking layer. The light-emitting auxiliary layer, the hole auxiliary layer, or the electron blocking layer may have an effect of improving the efficiency and/or lifetime of the organic electroluminescent device.

In addition, in an organic electroluminescent device of the present disclosure, a mixed region of an electron transport compound and a reductive dopant, or a mixed region of a hole transport compound and an oxidative dopant, may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferred oxidative dopants include various Lewis acids and acceptor compounds, and preferred reductive dopants include alkali metals, alkali metal compounds, alkaline earth metals, rare earth metals, and mixtures thereof. In addition, an organic electroluminescent device having at least two light-emitting layers and emitting white light may be manufactured by using the reductive dopant layer as a charge-generating layer.

The organic electroluminescent material according to one embodiment of the present disclosure may be used as a light-emitting material for a white organic light-emitting device. The white organic light-emitting device has been suggested to have various structures such as a side-by-side structure or a stacking structure depending on the arrangement of R (Red), G (Green) or YG (Yellowish Green), and B (Blue) light-emitting parts, or a color conversion material (CCM) method, etc. In addition, the organic electroluminescent material according to one embodiment of the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as inkjet printing, nozzle printing, slot coating, spin coating, dip coating, flow coating methods, etc., can be used. When forming a film of the first host compound and the second host compound of the present disclosure, co-deposition or mixed deposition is performed.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

In addition, it is possible to produce a display system, e.g. a display system for smartphones, tablets, notebooks, PCs, TVs, or cars; or a lighting system, e.g. an outdoor or indoor lighting system, using the organic electroluminescent device of the present disclosure.

Hereinafter, the preparation method of the compound of the present disclosure, and the properties thereof, and the driving voltage and the luminous efficiency of the organic electroluminescent device (OLED) comprising a plurality of host materials according to the present disclosure, will be explained in detail with reference to the representative compounds of the present disclosure. However, the following examples only describe the properties of the compound according to the present disclosure and the OLED comprising the same, and the present disclosure is not limited to the following examples.

### Example 1: Preparation of Compound H1-93

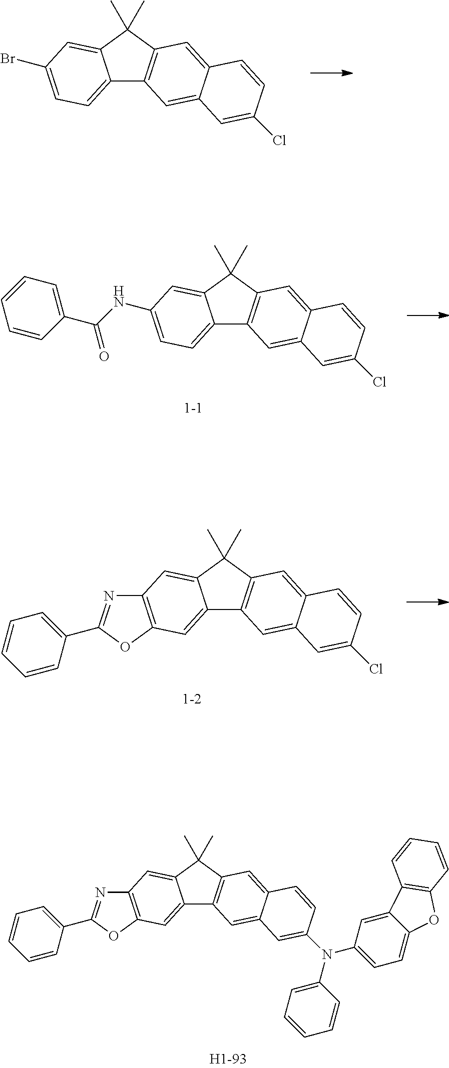 [SMILES: CC1(C)c2cc(Br)ccc2-c2cc3cc(Cl)ccc3cc21 ; CC1(C)c2cc(NC(=O)c3ccccc3)ccc2-c2cc3cc(Cl)ccc3cc21 ; CC1(C)c2cc3ccc(Cl)cc3cc2-c2cc3oc(-c4ccccc4)nc3cc21 ; CC1(C)c2cc3ccc(N(c4ccccc4)c4ccc5oc6ccccc6c5c4)cc3cc2-c2cc3oc(-c4ccccc4)nc3cc21]

### 1) Synthesis of Compound 1-1

2-bromo-7-chloro-11,11-dimethyl-11H-benzo[b]fluorene (50 g, 140 mmol), benzamide (17 g, 140 mmol), CuI (40 g, 210 mmol), ethylenediamine (28.3 mL, 419 mmol), K₃PO₄ (59 g, 280 mmol), 350 mL of toluene, and 350 mL of xylene were added into a flask followed by refluxing for 24 hours at 135° C. After completion of the reaction, the organic layer was extracted with ethyl acetate, and the remaining moisture was removed with magnesium sulfate. Thereafter, the organic layer was dried, and separated by column chromatography to obtain compound 1-1 (50 g, yield: 90%).

### 2) Synthesis of Compound 1-2

Compound 1-1 (10 g, 25 mmol) and Cu(Otf)₂ (13.6 g, 37 mmol) were dissolved in 312 mL of 1,2-DCB followed by refluxing for 48 hours at 170° C. After completion of the reaction, the organic layer was extracted with ethyl acetate, and the remaining moisture was removed with magnesium sulfate. Thereafter, the organic layer was dried, and separated by column chromatography to obtain compound 1-2 (2.4 g, yield: 24%).

### 3) Synthesis of Compound H1-93

Compound 1-2 (2.8 g, 7.0 mmol), N-phenyldibenzo[b,d]furan-2-amine (2.4 g, 9.0 mmol), Pd₂(dba)₃ (0.65 g, 0.7 mmol), s-phos (580 mg, 1.4 mmol), NaOtBu (1.7 g, 17 mmol) and 35 mL of xylene were added into a flask followed by stirring under reflux for 2 hours. After completion of the reaction, the organic layer was extracted with ethyl acetate, and the remaining moisture was removed with magnesium sulfate. Thereafter, the organic layer was dried, and separated by column chromatography to obtain compound H1-93 (1.8 g, yield: 42%).

| | M.W. | M.P. |
|---|---|---|
| H1-93 | 618.74 | 237° C. |

### Example 2: Preparation of Compound H1-10

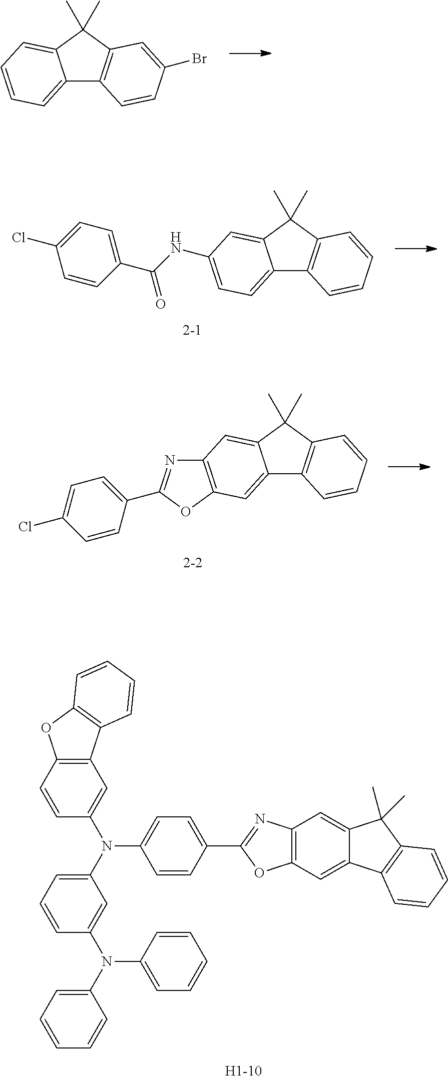 [SMILES: CC1(C)c2ccccc2-c2ccc(Br)cc21 ; CC1(C)c2ccccc2-c2ccc(NC(=O)c3ccc(Cl)cc3)cc21 ; CC1(C)c2ccccc2-c2cc3oc(-c4ccc(Cl)cc4)nc3cc21 ; CC1(C)c2ccccc2-c2cc3oc(-c4ccc(N(c5cccc(N(c6ccccc6)c6ccccc6)c5)c5ccc6oc7ccccc7c6c5)cc4)nc3cc21]

### 1) Synthesis of Compound 2-1

2-bromo-7-chloro-9,9-dimethyl-9H-fluorene (50 g, 183 mmol), 4-chlorobenzamide (28.5 g, 183 mmol), CuI (52.3 g, 274 mmol), ethylenediamine (37 mL, 549 mmol), K₃PO₄ (77.7 g, 366 mmol), and 915 mL of xylene were added into a flask followed by refluxing for 24 hours at 150° C. After completion of the reaction, the organic layer was extracted with ethyl acetate, and the remaining moisture was removed with magnesium sulfate. Thereafter, the organic layer was dried, and separated by column chromatography to obtain compound 2-1 (42.6 g, yield: 67%).

### 2) Synthesis of Compound 2-2

Compound 2-1 (15 g, 43 mmol) and Cu(Otf)₂ (23 g, 65 mmol) were dissolved in 540 mL of 1,2-DCB followed by refluxing for 48 hours at 170° C. After completion of the reaction, the organic layer was extracted with ethyl acetate, and the remaining moisture was removed with magnesium sulfate. Thereafter, the organic layer was dried, and separated by column chromatography to obtain compound 2-2 (3.6 g, yield: 24%).

### 3) Synthesis of Compound H1-10

Compound 2-2 (2.0 g, 5.7 mmol), N¹-(dibenzo[b,d]furan-2-yl)-N³,N³-diphenylbenzene-1,3-diamine (2.7 g, 6.3 mmol), Pd₂(dba)₃ (0.26 g, 0.28 mmol), s-phos (237 mg, 10.57 mmol), NaOtBu (1.1 g, 11 mmol) and 57 mL of xylene were added into a flask followed by stirring under reflux for 1 hour. After completion of the reaction, the organic layer was extracted with ethyl acetate, and the remaining moisture was removed with magnesium sulfate. Thereafter, the organic layer was dried, and separated by column chromatography to obtain compound H1-10 (1.2 g, yield: 28%).

| | M.W. | M.P. |
|---|---|---|
| H1-10 | 735.89 | 236° C. |

### Example 3: Preparation of Compound H1-9

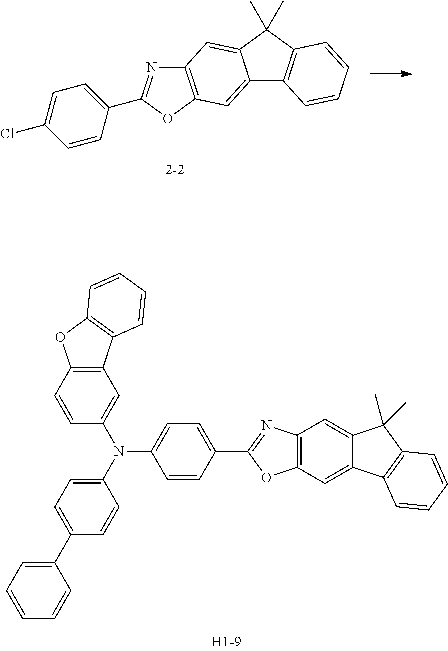 [SMILES: CC1(C)c2ccccc2-c2cc3oc(-c4ccc(Cl)cc4)nc3cc21 ; CC1(C)c2ccccc2-c2cc3oc(-c4ccc(N(c5ccc(-c6ccccc6)cc5)c5ccc6oc7ccccc7c6c5)cc4)nc3cc21]

### 1) Synthesis of Compound H1-9

Compound 2-2 (1.0 g, 2.9 mmol), N-([1,1′-biphenyl]-4-yl)dibenzo[b,d]furan-2-amine (1.1 g, 3.2 mmol), Pd₂(dba)₃ (0.13 g, 0.14 mmol), s-phos (119 mg, 0.28 mmol), NaOtBu (0.55 g, 5.8 mmol) and 29 mL of xylene were added into a flask followed by stirring under reflux for 1 hour. After completion of the reaction, the organic layer was extracted with ethyl acetate, and the remaining moisture was removed with magnesium sulfate. Thereafter, the organic layer was dried, and separated by column chromatography to obtain compound H1-9 (1.0 g, yield: 55%).

| | M.W. | M.P. |
|---|---|---|
| H1-9 | 644.77 | 151.2° C. |

### Example 4: Preparation of Compound H1-3

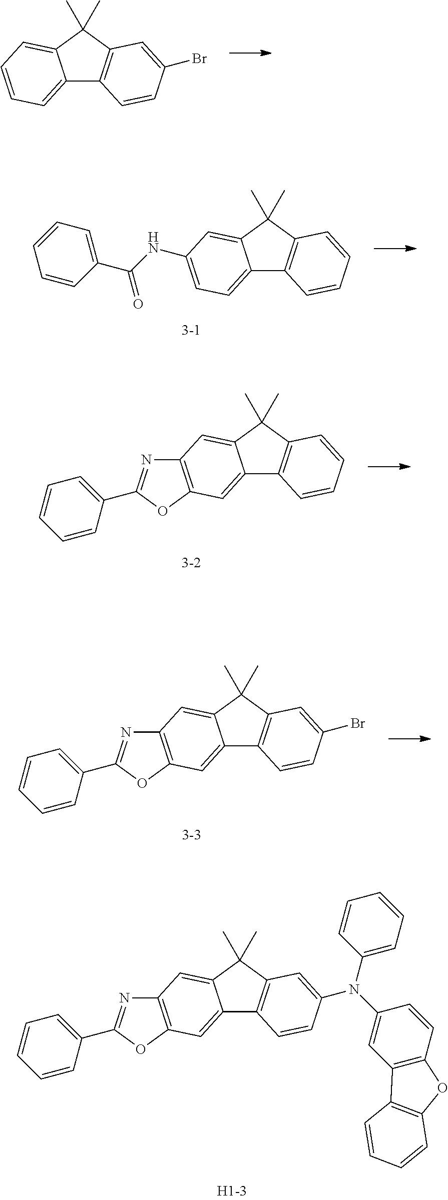 [SMILES: CC1(C)c2ccccc2-c2ccc(Br)cc21 ; CC1(C)c2ccccc2-c2ccc(NC(=O)c3ccccc3)cc21 ; CC1(C)c2ccccc2-c2cc3oc(-c4ccccc4)nc3cc21 ; CC1(C)c2cc(Br)ccc2-c2cc3oc(-c4ccccc4)nc3cc21 ; CC1(C)c2cc(N(c3ccccc3)c3ccc4oc5ccccc5c4c3)ccc2-c2cc3oc(-c4ccccc4)nc3cc21]

### 1) Synthesis of Compound 3-1

2-bromo-7-chloro-9,9-dimethyl-9H-fluorene (30 g, 110 mmol), benzamide (13.3 g, 110 mmol), CuI (31.3 g, 165 mmol), ethylenediamine (22.2 mL, 330 mmol), K₃PO₄ (46.6 g, 220 mmol), and 550 mL of xylene were added into a flask followed by refluxing for 24 hours at 150° C. After completion of the reaction, the organic layer was extracted with ethyl acetate, and the remaining moisture was removed with magnesium sulfate. Thereafter, the organic layer was dried, and separated by column chromatography to obtain compound 3-1 (26.4 g, yield: 77%).

### 2) Synthesis of Compound 3-2

Compound 3-1 (25 g, 80 mmol) and Cu(Otf)₂ (43.2 g, 120 mmol) were dissolved in 1000 mL of 1,2-DCB followed by refluxing for 24 hours at 170° C. After completion of the reaction, the organic layer was extracted with ethyl acetate, and the remaining moisture was removed with magnesium sulfate. Thereafter, the organic layer was dried, and separated by column chromatography to obtain compound 3-2 (5.1 g, yield: 20%).

### 3) Synthesis of Compound 3-3

Compound 3-2 (5 g, 16 mmol) and NBS (5.7 g, 32 mmol) were dissolved in 160 mL of DMF followed by refluxing for 5 hours at 100° C. After completion of the reaction, the organic layer was extracted with ethyl acetate, and the remaining moisture was removed with magnesium sulfate. Thereafter, the organic layer was dried, and separated by column chromatography to obtain compound 3-3 (5.0 g, yield: 79%).

### 4) Synthesis of Compound H1-3

Compound 3-3 (2.5 g, 6.4 mmol), N-phenyldibenzo[b,d]furan-2-amine (1.7 g, 6.7 mmol), Pd₂(dba)₃ (0.29 g, 0.32 mmol), s-phos (263 mg, 0.64 mmol), NaOtBu (1.2 g, 12 mmol) and 64 mL of xylene were added into a flask followed by stirring under reflux for 3 hours. After completion of the reaction, the organic layer was extracted with ethyl acetate, and the remaining moisture was removed with magnesium sulfate. Thereafter, the organic layer was dried, and separated by column chromatography to obtain compound H1-3 (2.8 g, yield: 77%).

| | M.W. | M.P. |
|---|---|---|
| H1-3 | 568.68 | 235.4° C. |

### Device Examples 1 and 2: Producing OLEDs by Co-Depositing the First Host Compound and the Second Host Compound According to the Present Disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1, shown in Table 3, was introduced into a cell of the vacuum vapor deposition apparatus, and compound HT-1 was introduced into another cell. The two materials were evaporated at different rates, and compound HI-1 was deposited in a doping amount of 3 wt % based to the total amount of compound HI-1 and compound HT-1 to form a first hole injection layer with a thickness of 10 nm. Subsequently, compound HT-1 was deposited on the first hole injection layer to form a first hole transport layer with a thickness of 80 nm. Next, compound HT-2 was introduced into another cell of the vacuum vapor deposition apparatus and was evaporated by applying an electric current to the cell, thereby depositing a second hole transport layer with a thickness of 60 nm on the first hole transport layer. After forming the hole injection layer and the hole transport layers, a light-emitting layer was deposited thereon as follows: each of the first host compound and the second host compound shown in Table 1 below were introduced into two cells of the vacuum vapor deposition apparatus as hosts, and compound D-39 was introduced into another cell as a dopant. The two host materials were evaporated at a rate of 1:1, the dopant material was simultaneously evaporated at a different rate, and the dopant was deposited in a doping amount of 3 wt % based on the total amount of the hosts and dopant to form a light-emitting layer with a thickness of 40 nm on the second hole transport layer. Then, compound ET-1 and compound EI-1 were evaporated at a weight ratio of 50:50 as an electron transport material to form an electron transport layer having a thickness of 35 nm on the light-emitting layer. After depositing compound EI-1 as an electron injection layer with a thickness of 2 nm on the electron transport layer, an Al cathode was deposited with a thickness of 80 nm on the electron injection layer by using another vacuum vapor deposition apparatus, thereby producing an OLED. All the materials used for producing the OLED were purified by vacuum sublimation at 10⁻⁶ torr.

### Comparative Example 1: Producing an OLED Comprising a Single Compound as a Host

An OLED was produced in the same manner as in Device Example 1, except that the second host compound shown in Table 1 below was used alone as a host of a light-emitting layer.

Table 1, below, shows the driving voltage, luminous efficiency, light-emitting color at a luminance of 5,000 nit, and time taken for luminance to reduce from 100% to 95% at a luminance of 10,000 nit (lifespan: T95) for the OLEDs of Device Examples 1, 2, and Comparative Example 1 produced as described above.

TABLE 1

| | | | Driving | Luminous | Light- | |
|---|---|---|---|---|---|---|
| | First | Second | Voltage | Efficiency | Emitting | Lifespan |
| | Host | Host | (V) | (cd/A) | Color | T95(hr) |
| Device Example 1 | H1-93 | H2-42 | 4.5 | 32.1 | Red | 31 |
| Device Example 2 | H1-10 | H2-42 | 4.6 | 32.6 | Red | 64 |
| Comparative | — | H2-42 | 4.5 | 26.7 | Red | 25 |
| Example 1 | | | | | | |

From Table 1 above, it can be confirmed that the OLED comprising the specific combination of compounds according to the present disclosure as a host material (Device Example 1 and 2), exhibits higher luminous efficiency and/or extended lifespan properties compared to the OLED comprising a single host material (Comparative Example 1).

### Device Example 3: Producing an OLED by Depositing the Second Hole Transport Layer Compound According to the Present Disclosure

An OLED was produced in the same manner as in Device Example 1, except that the second hole transport layer compound, shown in Table 2 below was used; and compound H3 and compound H2-42 were used as each of the first host compound and each of the second host compound as the host of the light-emitting layer.

### Comparative Example 2: Producing an OLED Comprising a Comparative Compound as a Second Hole Transport Layer

An OLED was produced in the same manner as in Device Example 3, except that the second hole transport layer compound shown in Table 2 was used.

Table 2, below, shows the driving voltage, luminous efficiency, light-emitting color at a luminance of 1,000 nit, and time taken for luminance to reduce from 100% to 95% at a luminance of 10,000 nit (lifespan: T95) for the OLEDs of Device Example 3 and Comparative Example 2 produced as described above.

TABLE 2

| | Second Hole | | | | |
|---|---|---|---|---|---|
| | Transport | Driving | Luminous | Light- | Life- |
| | Layer | Voltage | Efficiency | Emitting | span |
| | Compound | (V) | (cd/A) | Color | T95(hr) |
| Device | H1-10 | 4.3 | 33.6 | Red | 229 |
| Example 3 | | | | | |
| Comparative | H1-9 | 4.4 | 32.8 | Red | 136 |
| Example 2 | | | | | |

From Table 2 above, it can be confirmed that the OLED comprising the compound according to the present disclosure in the second hole transport layer (Device Example 3) exhibits a driving voltage and luminous efficiency characteristics at an equivalent level, as well as significantly improved lifetime properties, compared to the OLED comprising a conventional compound (Comparative Example 2).

TABLE 3

| | |
|---|---|
| Hole Injection Layer/ Hole Transport Layer | 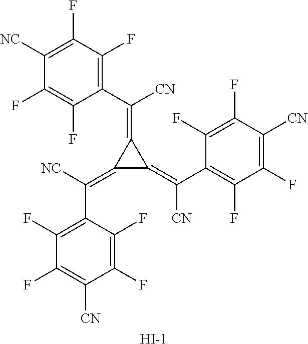 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 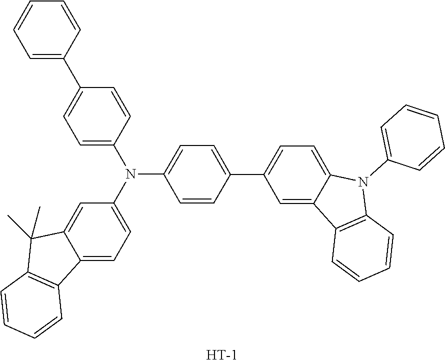 |
| | 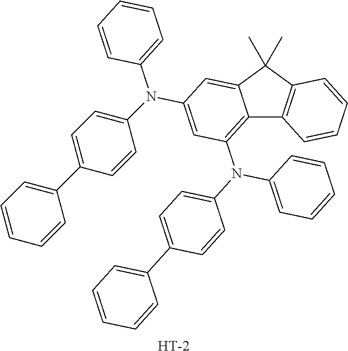 |
| Light-Emitting Layer/ Hole Transport Zone | 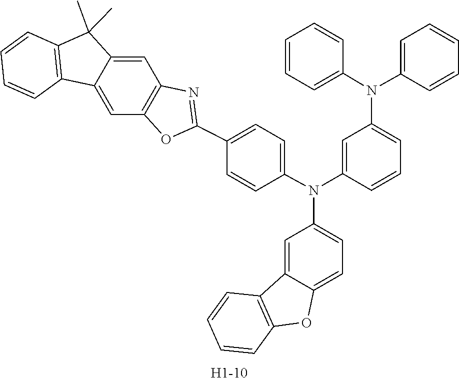 [SMILES: CC1(C)c2ccccc2-c2cc3oc(-c4ccc(N(c5cccc(N(c6ccccc6)c6ccccc6)c5)c5ccc6oc7ccccc7c6c5)cc4)nc3cc21] |
| | 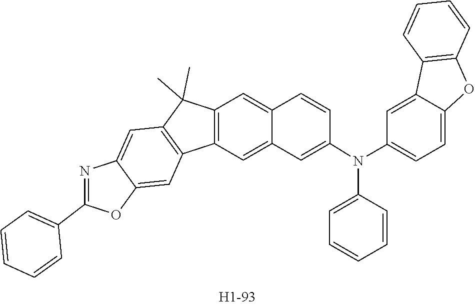 [SMILES: CC1(C)c2cc3ccc(N(c4ccccc4)c4ccc5oc6ccccc6c5c4)cc3cc2-c2cc3oc(-c4ccccc4)nc3cc21] |
| | 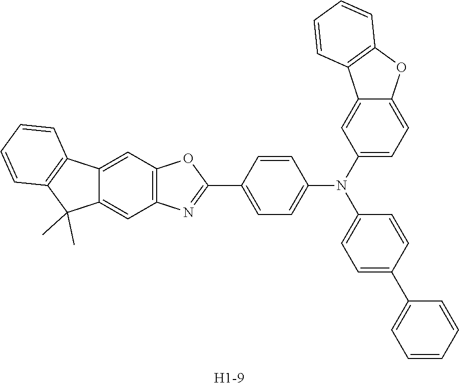 [SMILES: CC1(C)c2ccccc2-c2cc3oc(-c4ccc(N(c5ccc(-c6ccccc6)cc5)c5ccc6oc7ccccc7c6c5)cc4)nc3cc21] |
| | 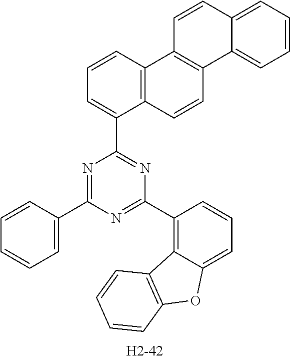 [SMILES: c1ccc(-c2nc(-c3cccc4c3ccc3c5ccccc5ccc43)nc(-c3cccc4oc5ccccc5c34)n2)cc1] |
| | 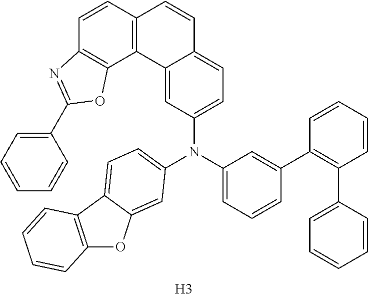 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1 ; "H3"] |
| | 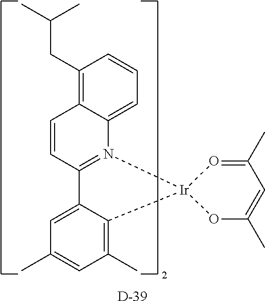 |
| Electron Transport Layer/Electron Injection Layer | 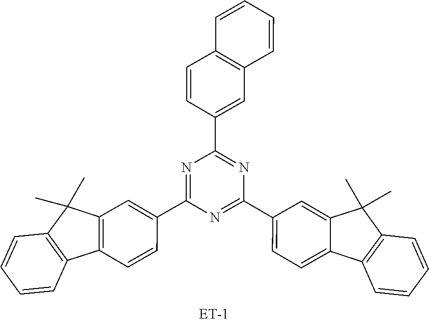 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 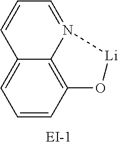 |

1. A plurality of host materials comprising a first host material comprising a compound represented by the following formula 1 and a second host material comprising a compound represented by the following formula 2: 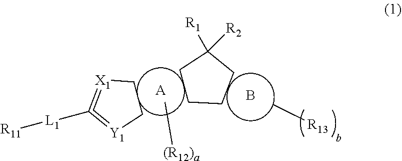
in formula 1,
R₁ and R₂ each independently represent a substituted or unsubstituted (C1-C30)alkyl, a substituted or unsubstituted (C6-C30)aryl, or a substituted or unsubstituted (3- to 30-membered)heteroaryl; or may be linked to each other to form a ring(s);
X₁ and Y₁ each independently represent —N═, —NR₁₄, —O— or —S—, with a proviso that any one of X₁ and Y₁ represents —N═, and the other of X₁ and Y₁ represents —NR₁₄—, —O— or —S—;
ring A and ring B each independently represent a substituted or unsubstituted (C6-C30)arene, or a substituted or unsubstituted (3- to 30-membered)heteroarene;
R₁₁ to R₁₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or the following formula 1-1, with a proviso that at least one of R₁₁ to R₁₄ is represented by the following formula 1-1; 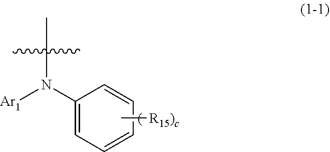
R₁₅ each independently represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₂-N(Ar₂)(Ar₃);
L₁ and L₂ each independently represent a single bond, a substituted or unsubstituted (C6-C30)arylene, or a substituted or unsubstituted (3- to 30-membered)heteroarylene;
Ar₁ to Ar₃ each independently represent hydrogen, deuterium, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; and
a represents an integer of 1 to 6, b represents an integer of 1 to 8, c represents an integer of 1 to 5, where if a to c are an integer of 2 or more, each of R₁₂, each of R₁₃, and each of R₁₅ may be the same as, or different from, each other. 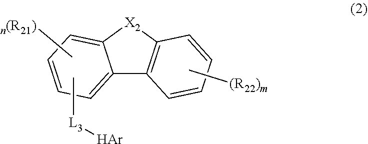
In formula 2,
X₂ represents —O— or —S—;
R₂₁ and R₂₂ each independently represent hydrogen, deuterium, or a substituted or unsubstituted (C6-C30)aryl;
L₃ represents a single bond, or a substituted or unsubstituted (C6-C30)arylene;
HAr represents a substituted or unsubstituted (3- to 30-membered)heteroaryl comprising at least one N atom; and
n represents an integer of 1 to 3, m represents an integer of 1 to 4, where if n and m are an integer of 2 or more, each of R₂₁ and each of R₂₂ may be the same as, or different from, each other.

2. The plurality of host materials according to claim 1, wherein the substituent(s) of the substituted alkyl, the substituted aryl(ene), the substituted heteroaryl(ene), the substituted arene, the substituted heteroarene, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), and the substituted alkenyl each independently are at least one selected from the group consisting of deuterium, a halogen, a cyano, a carboxyl, a nitro, a hydroxy, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (5- to 30-membered)heteroaryl unsubstituted or substituted with a (C6-C30)aryl(s), a (C6-C30)aryl unsubstituted or substituted with a (5- to 30-membered)heteroaryl(s), a tri(C1-C30)alkylsilyl, a tri(C6-C30)arylsilyl, a di(C1-C30)alkyl(C6-C30)arylsilyl, a (C1-C30)alkyldi(C6-C30)arylsilyl, a fused ring group of a (C3-C30) aliphatic ring(s) and a (C6-C30) aromatic ring(s), an amino, a mono- or di-(C1-C30)alkylamino, a mono- or di-(C2-C30)alkenylamino, a (C1-C30)alkyl(C2-C30)alkenylamino, a substituted or unsubstituted mono- or di-(C6-C30)arylamino, a (C1-C30)alkyl(C6-C30)arylamino, a mono- or di-(3- to 30-membered)heteroarylamino, a (C1-C30)alkyl(3- to 30-membered)heteroarylamino, a (C2-C30)alkenyl(C6-C30)arylamino, a (C2-C30)alkenyl(3- to 30-membered)heteroarylamino, a (C6-C30)aryl(3- to 30-membered)heteroarylamino, a (C1-C30)alkylcarbonyl, a (C1-C30)alkoxycarbonyl, a (C6-C30)arylcarbonyl, a (C6-C30)arylphospinyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

3. The plurality of host materials according to claim 1, wherein formula 1 is represented by one of the following formulas 3 and 4: 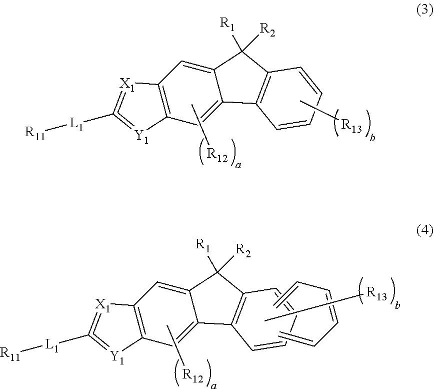
in formulas 3 and 4,
R₁, R₂, R₁₁ to R₁₃, X₁, Y₁, and L₁, are as defined in claim 1, a represents an integer of 1 or 2, and b represents an integer of 1 to 6.

4. The plurality of host materials according to claim 1, wherein formula 1 is represented by one of the following formulas 5 to 8: 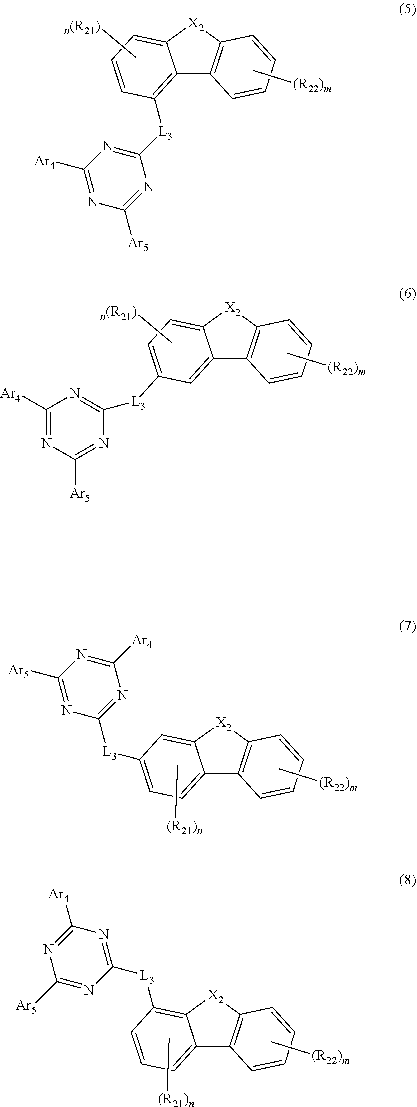
in formulas 5 to 8,
Ar₄ and Ar₅ each independently represent a substituted or unsubstituted (C6-C30)aryl, or a substituted or unsubstituted (3- to 30-membered)heteroaryl;
X₂, R₂₁ and R₂₂, L₃, n, m are as defined in claim 1.

5. The plurality of host materials according to claim 1, wherein the compound represented by formula 1 is at least one selected from the following compounds:

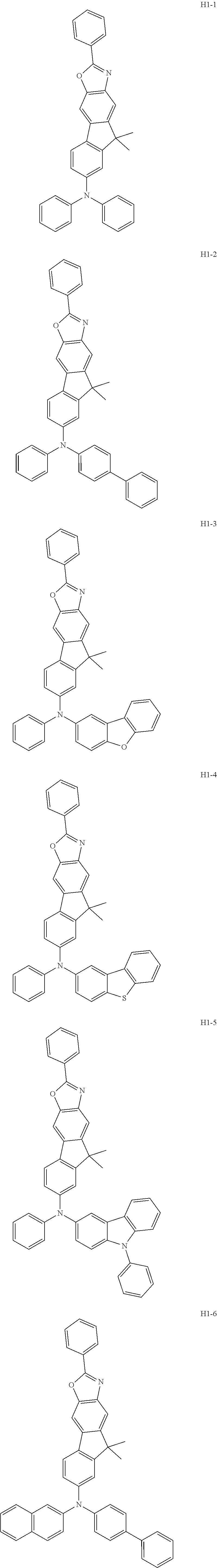 [SMILES: CC1(C)c2cc(N(c3ccccc3)c3ccccc3)ccc2-c2cc3oc(-c4ccccc4)nc3cc21 ; CC1(C)c2cc(N(c3ccccc3)c3ccc(-c4ccccc4)cc3)ccc2-c2cc3oc(-c4ccccc4)nc3cc21 ; CC1(C)c2cc(N(c3ccccc3)c3ccc4oc5ccccc5c4c3)ccc2-c2cc3oc(-c4ccccc4)nc3cc21 ; CC1(C)c2cc(N(c3ccccc3)c3ccc4sc5ccccc5c4c3)ccc2-c2cc3oc(-c4ccccc4)nc3cc21 ; CC1(C)c2cc(N(c3ccccc3)c3ccc4c(c3)c3ccccc3n4-c3ccccc3)ccc2-c2cc3oc(-c4ccccc4)nc3cc21 ; CC1(C)c2cc(N(c3ccc(-c4ccccc4)cc3)c3ccc4ccccc4c3)ccc2-c2cc3oc(-c4ccccc4)nc3cc21]

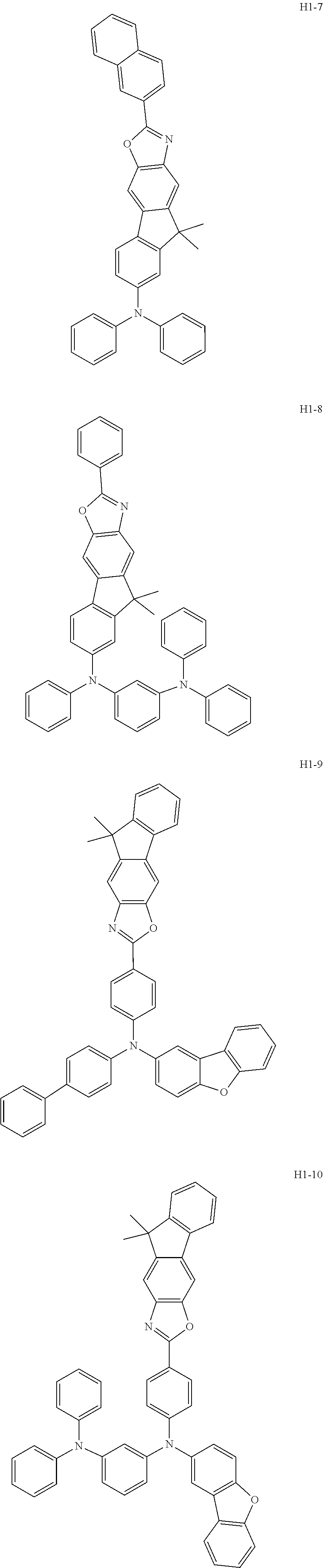 [SMILES: CC1(C)c2cc(N(c3ccccc3)c3ccccc3)ccc2-c2cc3oc(-c4ccc5ccccc5c4)nc3cc21 ; CC1(C)c2cc(N(c3ccccc3)c3cccc(N(c4ccccc4)c4ccccc4)c3)ccc2-c2cc3oc(-c4ccccc4)nc3cc21 ; CC1(C)c2ccccc2-c2cc3oc(-c4ccc(N(c5ccc(-c6ccccc6)cc5)c5ccc6oc7ccccc7c6c5)cc4)nc3cc21 ; CC1(C)c2ccccc2-c2cc3oc(-c4ccc(N(c5cccc(N(c6ccccc6)c6ccccc6)c5)c5ccc6oc7ccccc7c6c5)cc4)nc3cc21]

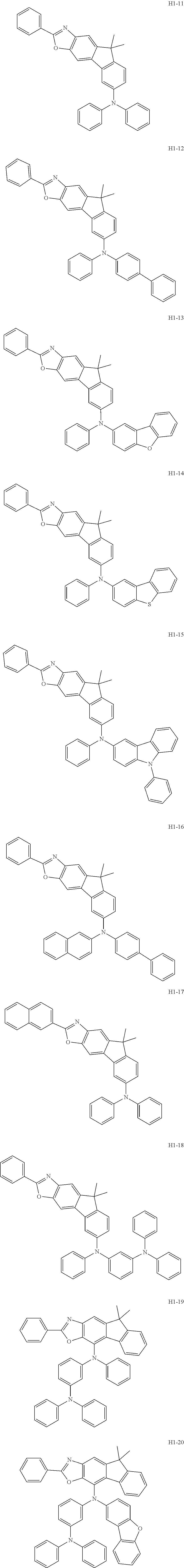

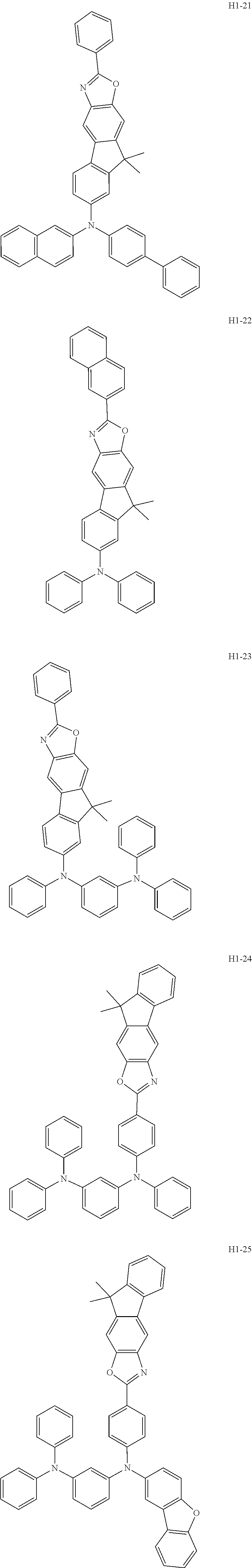 [SMILES: CC1(C)c2cc(N(c3ccc(-c4ccccc4)cc3)c3ccc4ccccc4c3)ccc2-c2cc3nc(-c4ccccc4)oc3cc21 ; CC1(C)c2cc(N(c3ccccc3)c3ccccc3)ccc2-c2cc3nc(-c4ccc5ccccc5c4)oc3cc21 ; CC1(C)c2cc(N(c3ccccc3)c3cccc(N(c4ccccc4)c4ccccc4)c3)ccc2-c2cc3nc(-c4ccccc4)oc3cc21 ; CC1(C)c2ccccc2-c2cc3nc(-c4ccc(N(c5ccccc5)c5cccc(N(c6ccccc6)c6ccccc6)c5)cc4)oc3cc21 ; CC1(C)c2ccccc2-c2cc3nc(-c4ccc(N(c5cccc(N(c6ccccc6)c6ccccc6)c5)c5ccc6oc7ccccc7c6c5)cc4)oc3cc21]

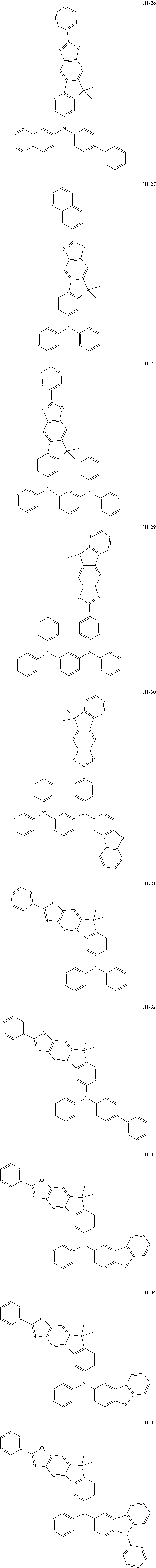

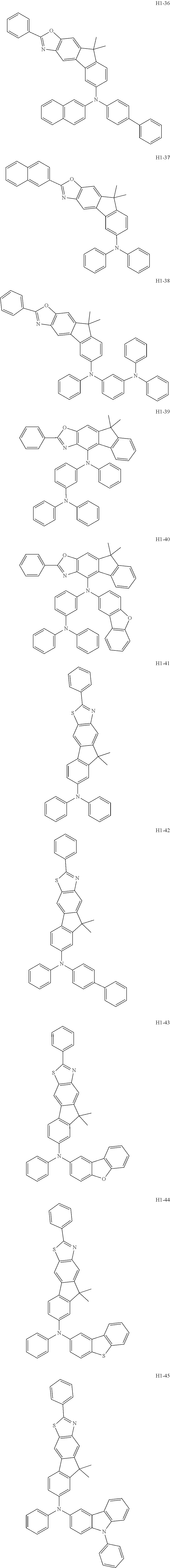

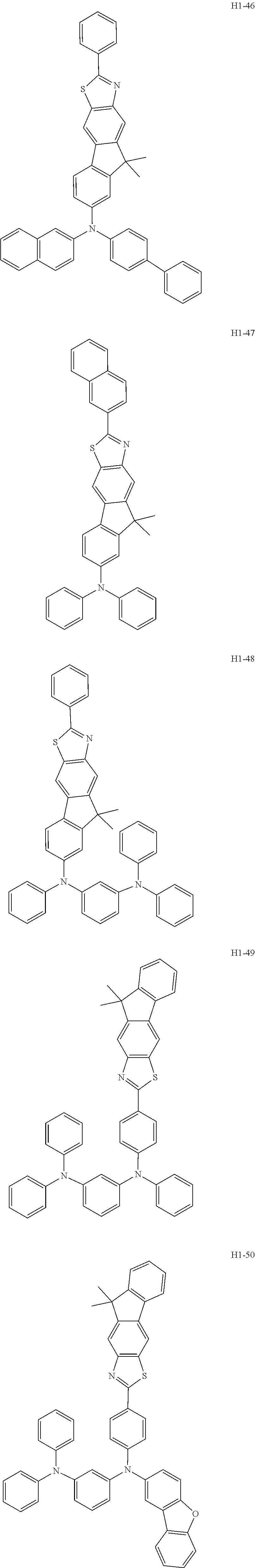 [SMILES: CC1(C)c2cc(N(c3ccc(-c4ccccc4)cc3)c3ccc4ccccc4c3)ccc2-c2cc3sc(-c4ccccc4)nc3cc21 ; CC1(C)c2cc(N(c3ccccc3)c3ccccc3)ccc2-c2cc3sc(-c4ccc5ccccc5c4)nc3cc21 ; CC1(C)c2cc(N(c3ccccc3)c3cccc(N(c4ccccc4)c4ccccc4)c3)ccc2-c2cc3sc(-c4ccccc4)nc3cc21 ; CC1(C)c2ccccc2-c2cc3sc(-c4ccc(N(c5ccccc5)c5cccc(N(c6ccccc6)c6ccccc6)c5)cc4)nc3cc21 ; CC1(C)c2ccccc2-c2cc3sc(-c4ccc(N(c5cccc(N(c6ccccc6)c6ccccc6)c5)c5ccc6oc7ccccc7c6c5)cc4)nc3cc21]

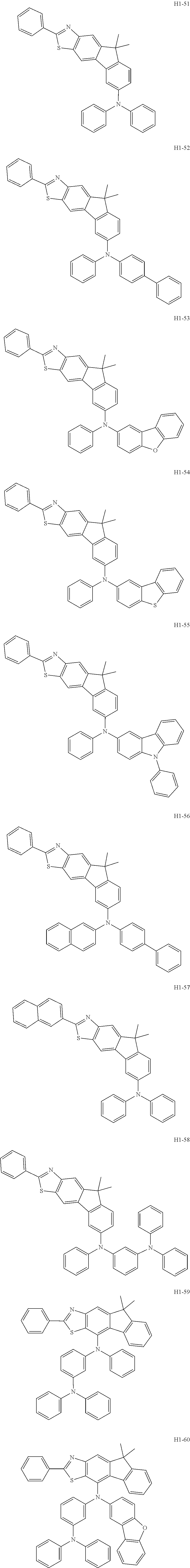

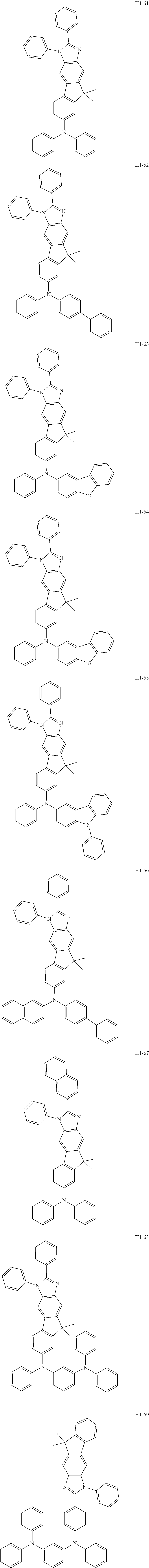

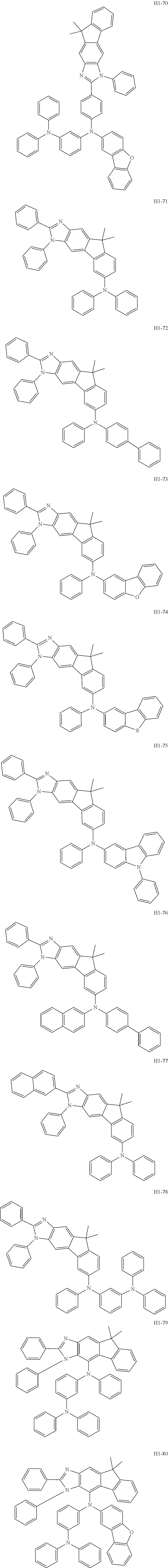

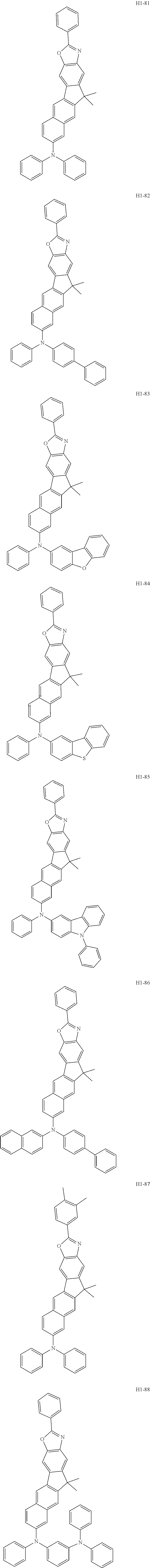

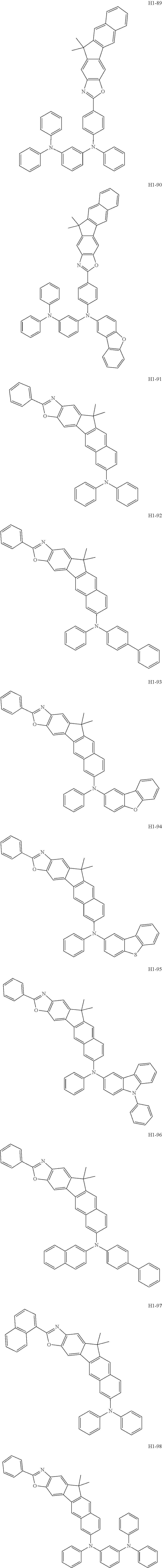

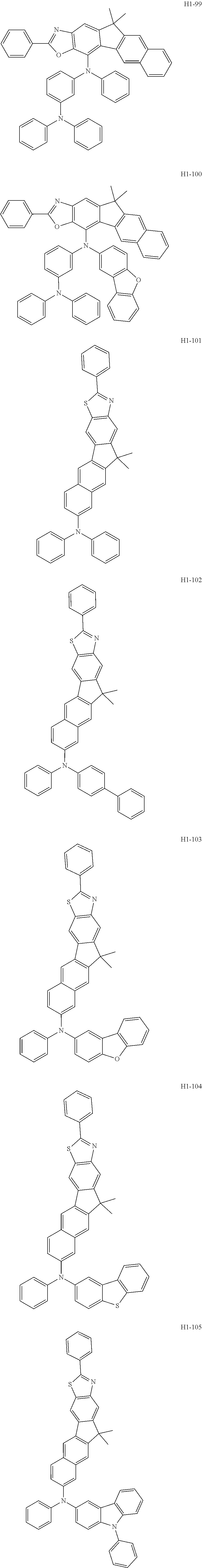

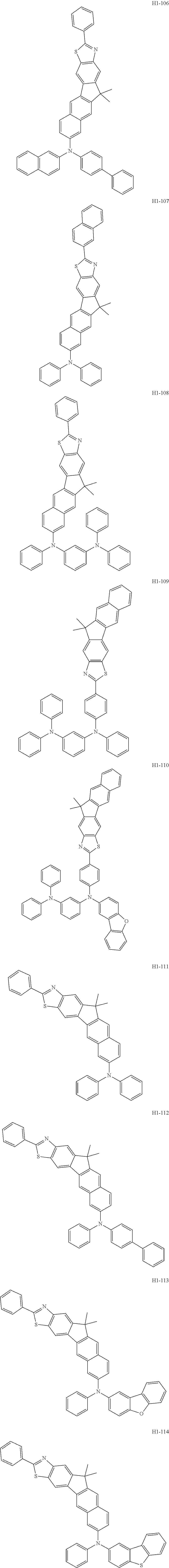

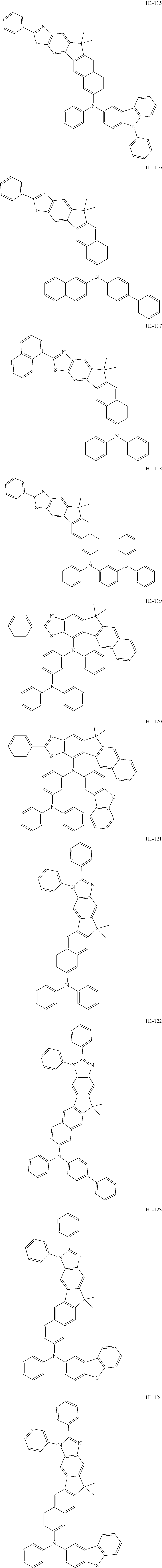

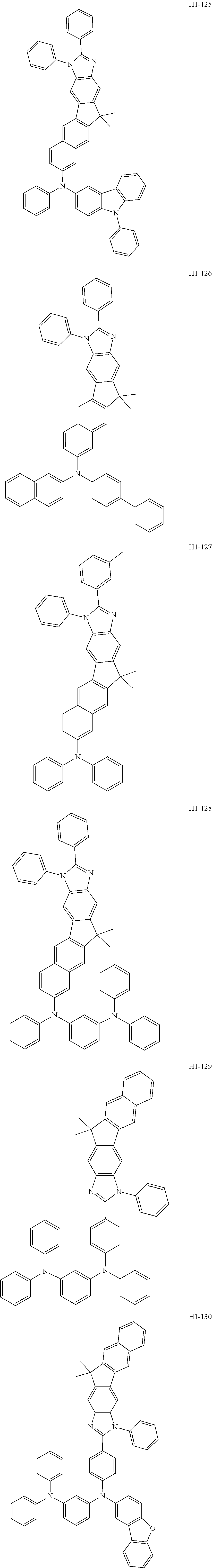

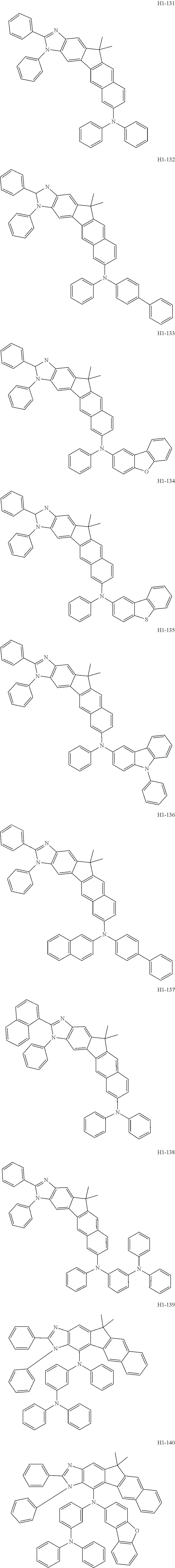

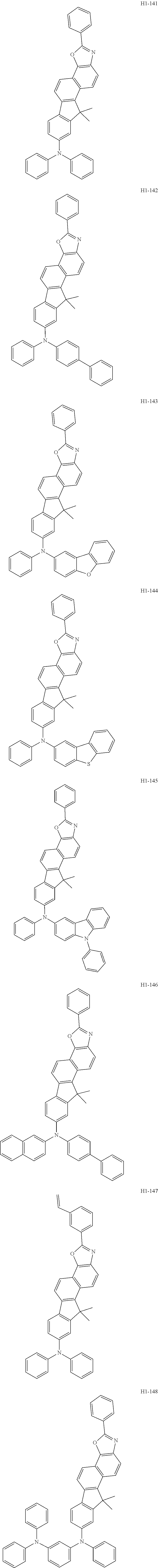

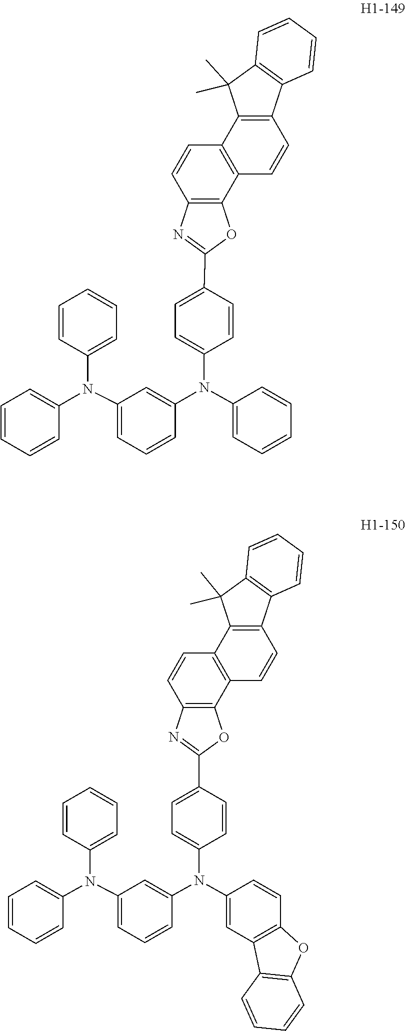 [SMILES: CC1(C)c2ccccc2-c2ccc3c(ccc4nc(-c5ccc(N(c6ccccc6)c6cccc(N(c7ccccc7)c7ccccc7)c6)cc5)oc43)c21 ; CC1(C)c2ccccc2-c2ccc3c(ccc4nc(-c5ccc(N(c6cccc(N(c7ccccc7)c7ccccc7)c6)c6ccc7oc8ccccc8c7c6)cc5)oc43)c21]

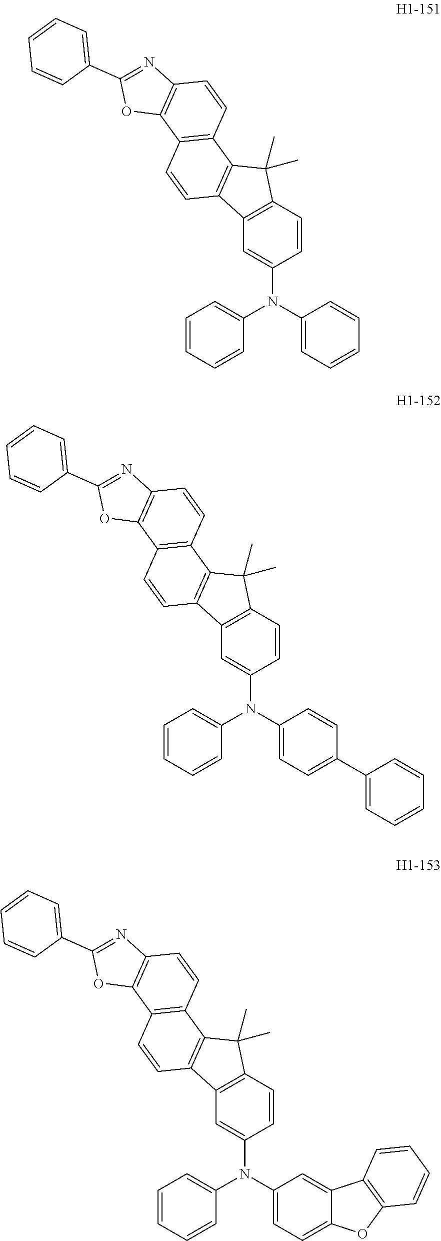 [SMILES: CC1(C)c2ccc(N(c3ccccc3)c3ccccc3)cc2-c2ccc3c(ccc4nc(-c5ccccc5)oc43)c21 ; CC1(C)c2ccc(N(c3ccccc3)c3ccc(-c4ccccc4)cc3)cc2-c2ccc3c(ccc4nc(-c5ccccc5)oc43)c21 ; CC1(C)c2ccc(N(c3ccccc3)c3ccc4oc5ccccc5c4c3)cc2-c2ccc3c(ccc4nc(-c5ccccc5)oc43)c21]

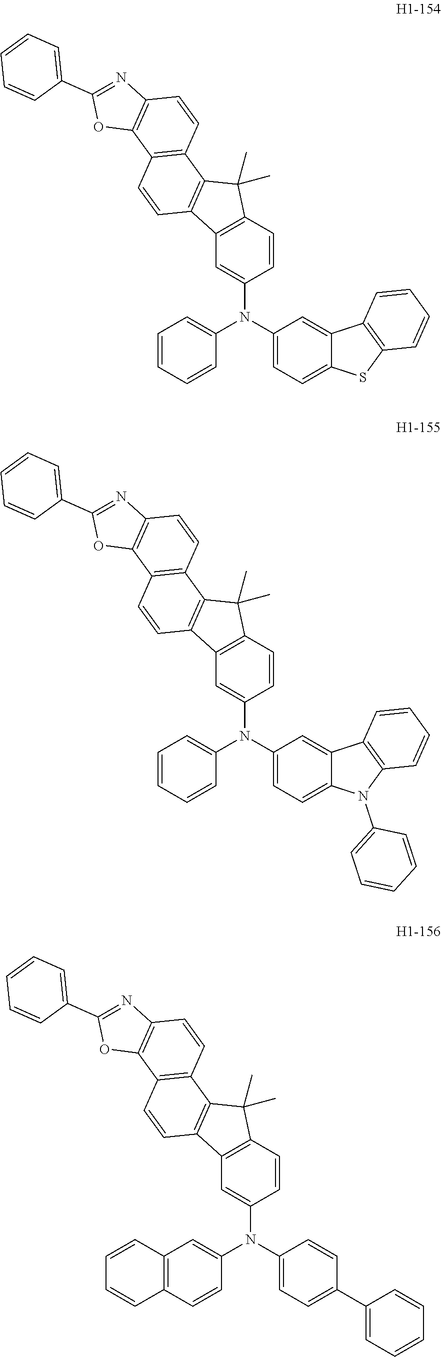 [SMILES: CC1(C)c2ccc(N(c3ccccc3)c3ccc4sc5ccccc5c4c3)cc2-c2ccc3c(ccc4nc(-c5ccccc5)oc43)c21 ; CC1(C)c2ccc(N(c3ccccc3)c3ccc4c(c3)c3ccccc3n4-c3ccccc3)cc2-c2ccc3c(ccc4nc(-c5ccccc5)oc43)c21 ; CC1(C)c2ccc(N(c3ccc(-c4ccccc4)cc3)c3ccc4ccccc4c3)cc2-c2ccc3c(ccc4nc(-c5ccccc5)oc43)c21]

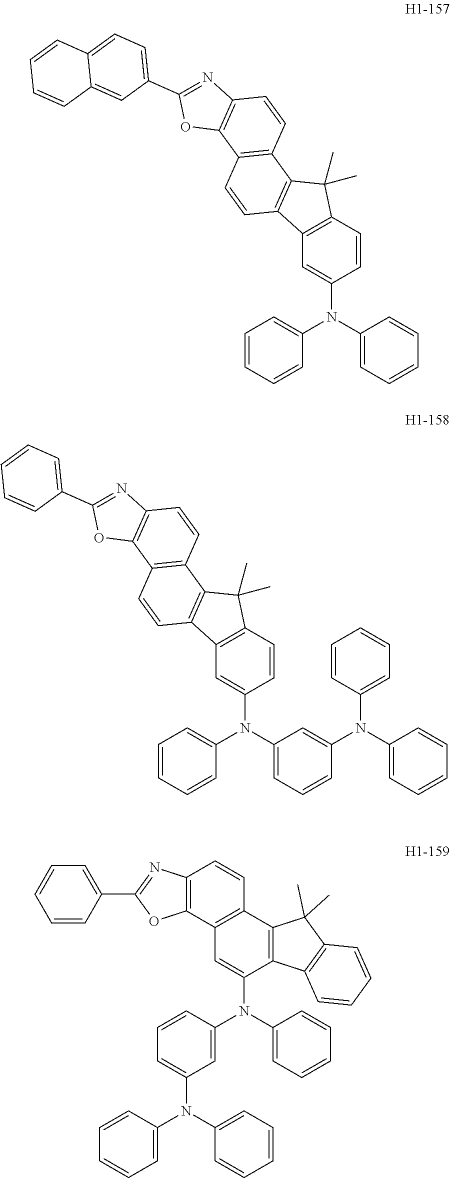 [SMILES: CC1(C)c2ccc(N(c3ccccc3)c3ccccc3)cc2-c2ccc3c(ccc4nc(-c5ccc6ccccc6c5)oc43)c21 ; CC1(C)c2ccc(N(c3ccccc3)c3cccc(N(c4ccccc4)c4ccccc4)c3)cc2-c2ccc3c(ccc4nc(-c5ccccc5)oc43)c21 ; CC1(C)c2ccccc2-c2c(N(c3ccccc3)c3cccc(N(c4ccccc4)c4ccccc4)c3)cc3c(ccc4nc(-c5ccccc5)oc43)c21]

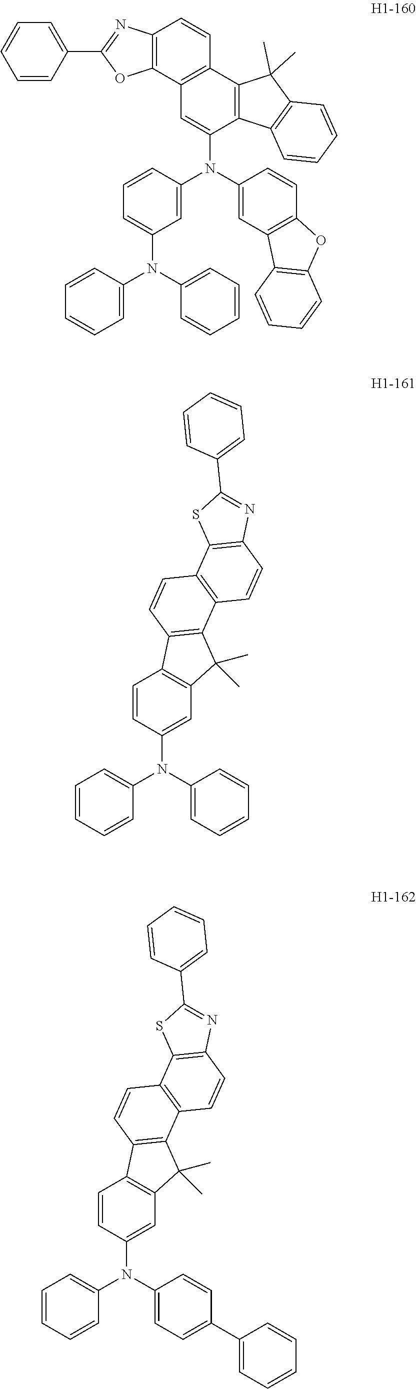 [SMILES: CC1(C)c2ccccc2-c2c(N(c3cccc(N(c4ccccc4)c4ccccc4)c3)c3ccc4oc5ccccc5c4c3)cc3c(ccc4nc(-c5ccccc5)oc43)c21 ; CC1(C)c2cc(N(c3ccccc3)c3ccccc3)ccc2-c2ccc3c(ccc4nc(-c5ccccc5)sc43)c21 ; CC1(C)c2cc(N(c3ccccc3)c3ccc(-c4ccccc4)cc3)ccc2-c2ccc3c(ccc4nc(-c5ccccc5)sc43)c21]

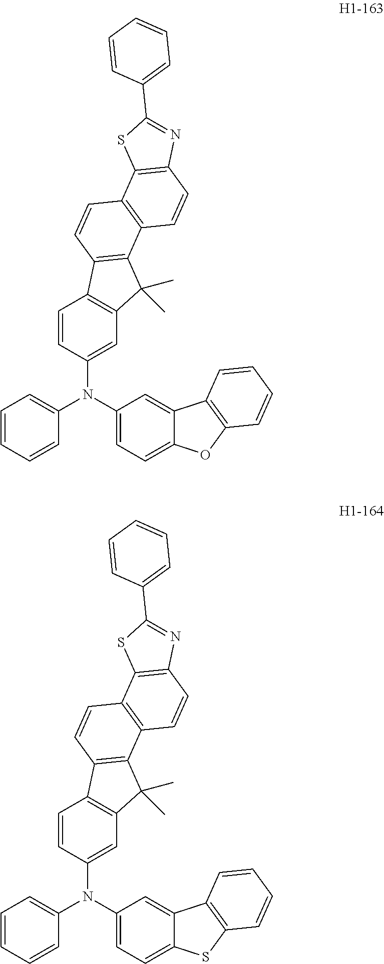 [SMILES: CC1(C)c2cc(N(c3ccccc3)c3ccc4oc5ccccc5c4c3)ccc2-c2ccc3c(ccc4nc(-c5ccccc5)sc43)c21 ; CC1(C)c2cc(N(c3ccccc3)c3ccc4sc5ccccc5c4c3)ccc2-c2ccc3c(ccc4nc(-c5ccccc5)sc43)c21]

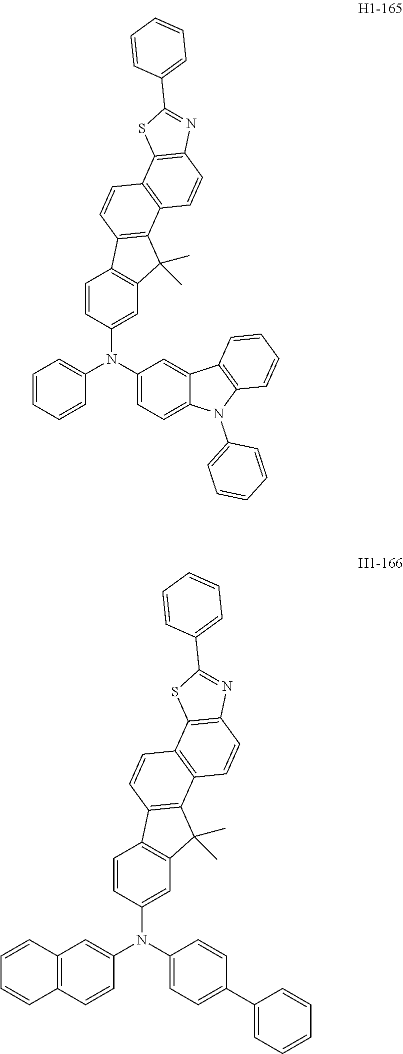 [SMILES: CC1(C)c2cc(N(c3ccccc3)c3ccc4c(c3)c3ccccc3n4-c3ccccc3)ccc2-c2ccc3c(ccc4nc(-c5ccccc5)sc43)c21 ; CC1(C)c2cc(N(c3ccc(-c4ccccc4)cc3)c3ccc4ccccc4c3)ccc2-c2ccc3c(ccc4nc(-c5ccccc5)sc43)c21]

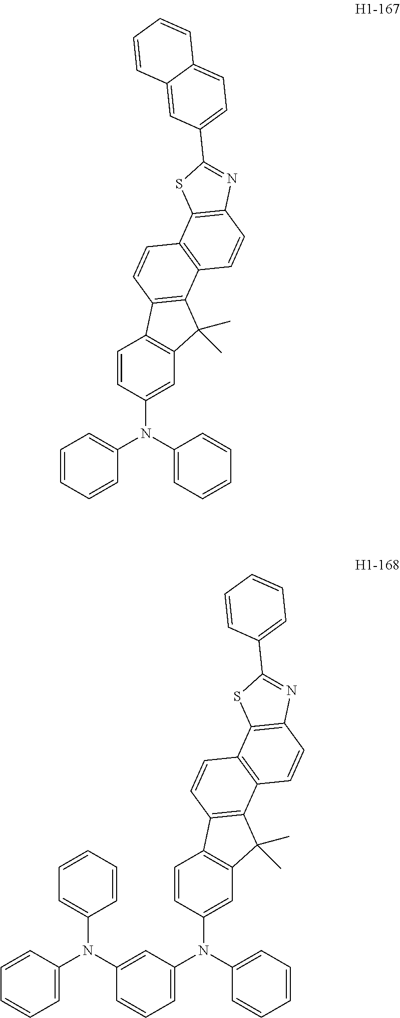 [SMILES: CC1(C)c2cc(N(c3ccccc3)c3ccccc3)ccc2-c2ccc3c(ccc4nc(-c5ccc6ccccc6c5)sc43)c21 ; CC1(C)c2cc(N(c3ccccc3)c3cccc(N(c4ccccc4)c4ccccc4)c3)ccc2-c2ccc3c(ccc4nc(-c5ccccc5)sc43)c21]

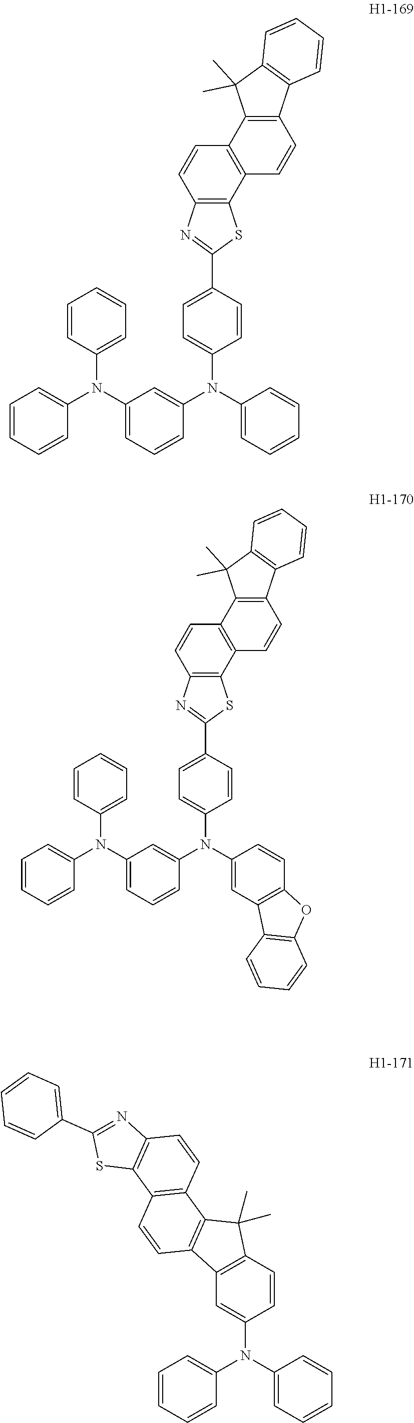 [SMILES: CC1(C)c2ccccc2-c2ccc3c(ccc4nc(-c5ccc(N(c6ccccc6)c6cccc(N(c7ccccc7)c7ccccc7)c6)cc5)sc43)c21 ; CC1(C)c2ccccc2-c2ccc3c(ccc4nc(-c5ccc(N(c6cccc(N(c7ccccc7)c7ccccc7)c6)c6ccc7oc8ccccc8c7c6)cc5)sc43)c21 ; CC1(C)c2ccc(N(c3ccccc3)c3ccccc3)cc2-c2ccc3c(ccc4nc(-c5ccccc5)sc43)c21]

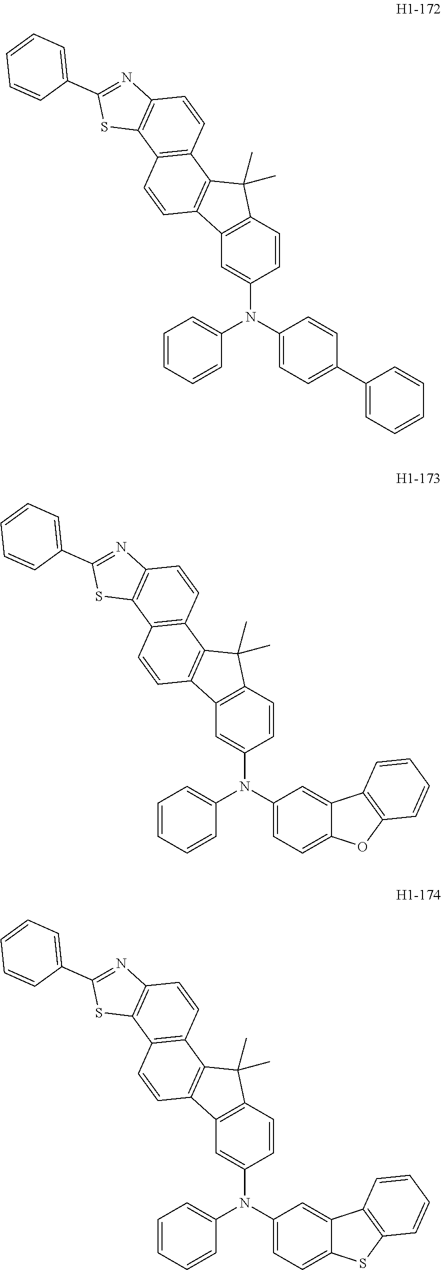 [SMILES: CC1(C)c2ccc(N(c3ccccc3)c3ccc(-c4ccccc4)cc3)cc2-c2ccc3c(ccc4nc(-c5ccccc5)sc43)c21 ; CC1(C)c2ccc(N(c3ccccc3)c3ccc4oc5ccccc5c4c3)cc2-c2ccc3c(ccc4nc(-c5ccccc5)sc43)c21 ; CC1(C)c2ccc(N(c3ccccc3)c3ccc4sc5ccccc5c4c3)cc2-c2ccc3c(ccc4nc(-c5ccccc5)sc43)c21]

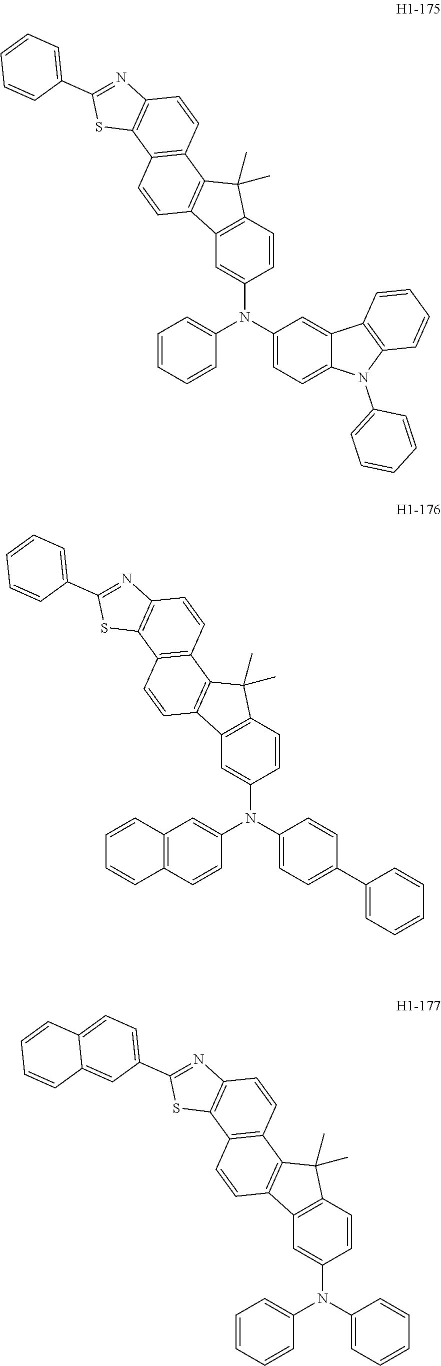 [SMILES: CC1(C)c2ccc(N(c3ccccc3)c3ccc4c(c3)c3ccccc3n4-c3ccccc3)cc2-c2ccc3c(ccc4nc(-c5ccccc5)sc43)c21 ; CC1(C)c2ccc(N(c3ccc(-c4ccccc4)cc3)c3ccc4ccccc4c3)cc2-c2ccc3c(ccc4nc(-c5ccccc5)sc43)c21 ; CC1(C)c2ccc(N(c3ccccc3)c3ccccc3)cc2-c2ccc3c(ccc4nc(-c5ccc6ccccc6c5)sc43)c21]

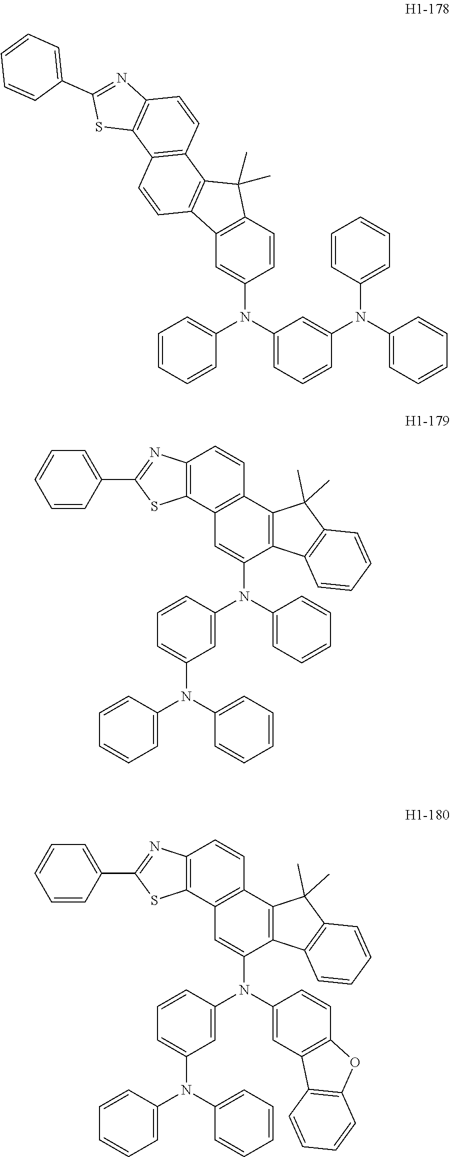 [SMILES: CC1(C)c2ccc(N(c3ccccc3)c3cccc(N(c4ccccc4)c4ccccc4)c3)cc2-c2ccc3c(ccc4nc(-c5ccccc5)sc43)c21 ; CC1(C)c2ccccc2-c2c(N(c3ccccc3)c3cccc(N(c4ccccc4)c4ccccc4)c3)cc3c(ccc4nc(-c5ccccc5)sc43)c21 ; CC1(C)c2ccccc2-c2c(N(c3cccc(N(c4ccccc4)c4ccccc4)c3)c3ccc4oc5ccccc5c4c3)cc3c(ccc4nc(-c5ccccc5)sc43)c21]

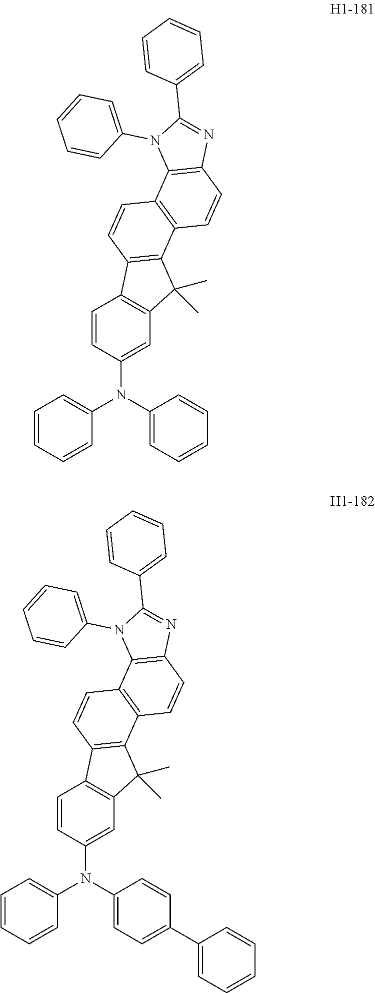 [SMILES: CC1(C)c2cc(N(c3ccccc3)c3ccccc3)ccc2-c2ccc3c(ccc4nc(-c5ccccc5)n(-c5ccccc5)c43)c21 ; CC1(C)c2cc(N(c3ccccc3)c3ccc(-c4ccccc4)cc3)ccc2-c2ccc3c(ccc4nc(-c5ccccc5)n(-c5ccccc5)c43)c21]

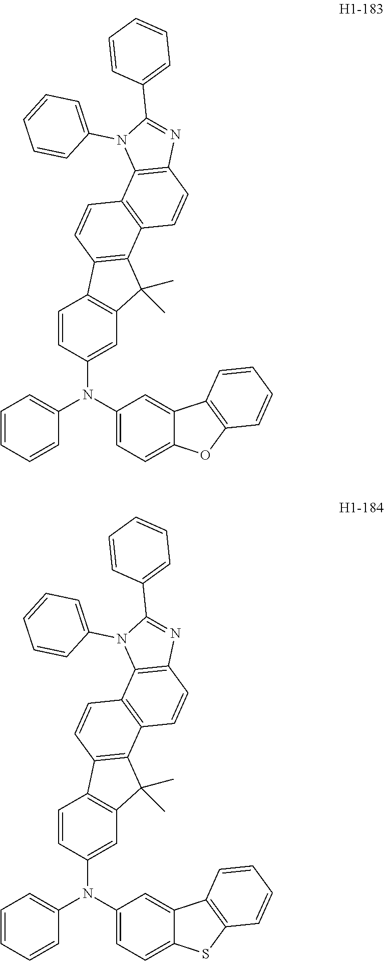 [SMILES: CC1(C)c2cc(N(c3ccccc3)c3ccc4oc5ccccc5c4c3)ccc2-c2ccc3c(ccc4nc(-c5ccccc5)n(-c5ccccc5)c43)c21 ; CC1(C)c2cc(N(c3ccccc3)c3ccc4sc5ccccc5c4c3)ccc2-c2ccc3c(ccc4nc(-c5ccccc5)n(-c5ccccc5)c43)c21]

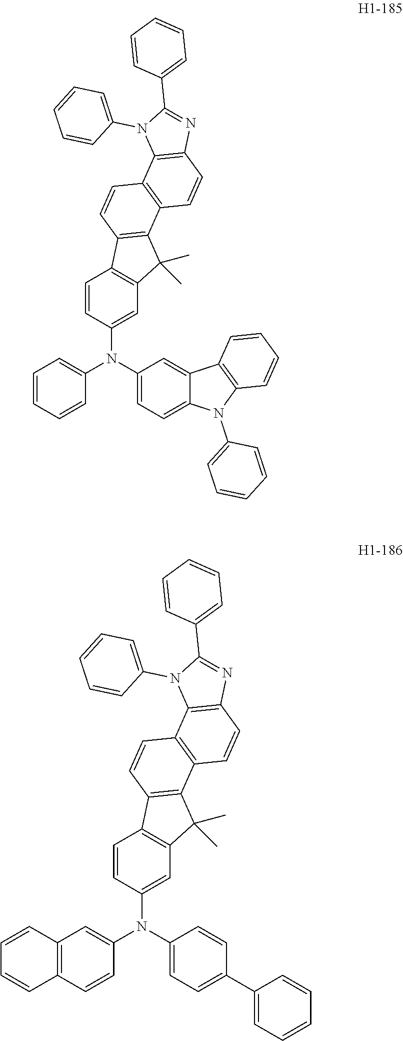 [SMILES: CC1(C)c2cc(N(c3ccccc3)c3ccc4c(c3)c3ccccc3n4-c3ccccc3)ccc2-c2ccc3c(ccc4nc(-c5ccccc5)n(-c5ccccc5)c43)c21 ; CC1(C)c2cc(N(c3ccc(-c4ccccc4)cc3)c3ccc4ccccc4c3)ccc2-c2ccc3c(ccc4nc(-c5ccccc5)n(-c5ccccc5)c43)c21]

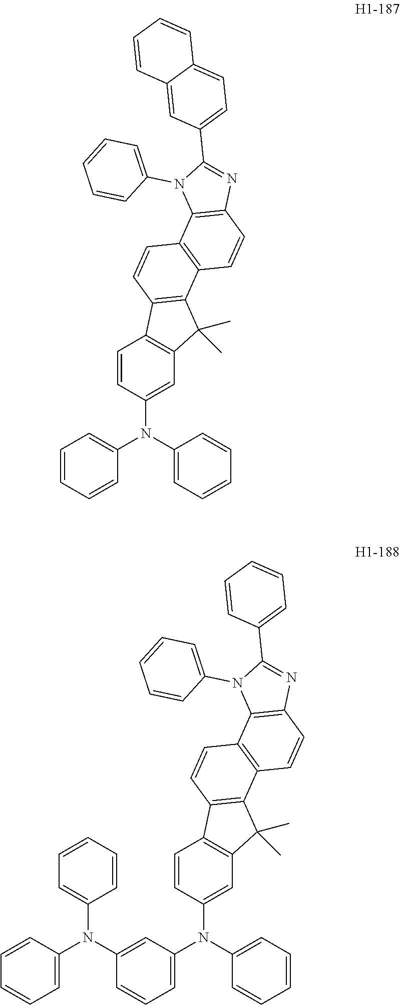 [SMILES: CC1(C)c2cc(N(c3ccccc3)c3ccccc3)ccc2-c2ccc3c(ccc4nc(-c5ccc6ccccc6c5)n(-c5ccccc5)c43)c21 ; CC1(C)c2cc(N(c3ccccc3)c3cccc(N(c4ccccc4)c4ccccc4)c3)ccc2-c2ccc3c(ccc4nc(-c5ccccc5)n(-c5ccccc5)c43)c21]

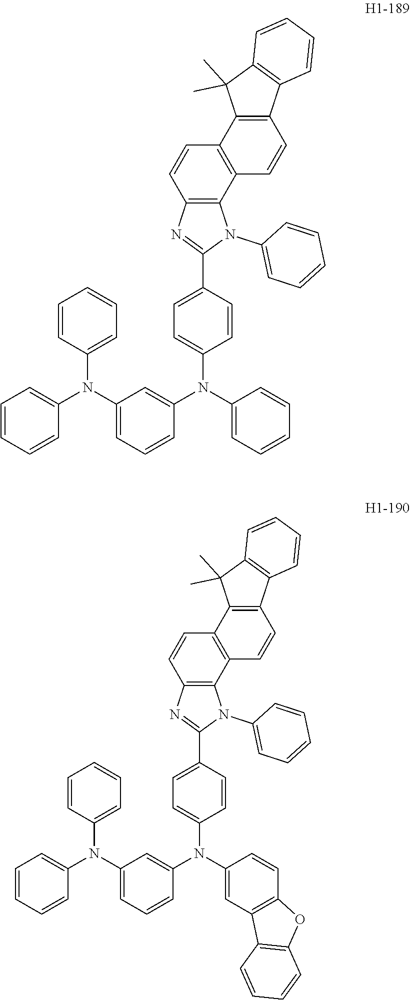 [SMILES: CC1(C)c2ccccc2-c2ccc3c(ccc4nc(-c5ccc(N(c6ccccc6)c6cccc(N(c7ccccc7)c7ccccc7)c6)cc5)n(-c5ccccc5)c43)c21 ; CC1(C)c2ccccc2-c2ccc3c(ccc4nc(-c5ccc(N(c6cccc(N(c7ccccc7)c7ccccc7)c6)c6ccc7oc8ccccc8c7c6)cc5)n(-c5ccccc5)c43)c21]

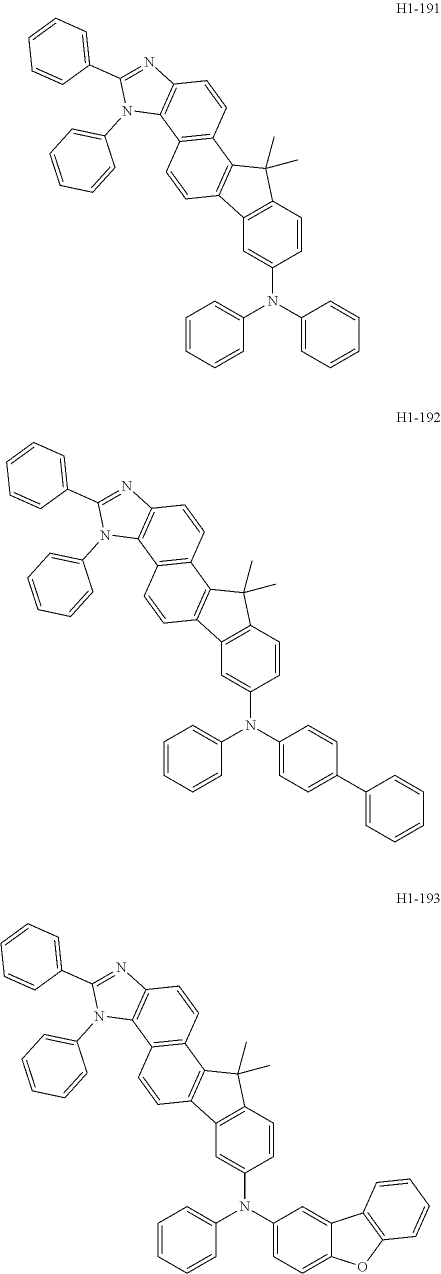 [SMILES: CC1(C)c2ccc(N(c3ccccc3)c3ccccc3)cc2-c2ccc3c(ccc4nc(-c5ccccc5)n(-c5ccccc5)c43)c21 ; CC1(C)c2ccc(N(c3ccccc3)c3ccc(-c4ccccc4)cc3)cc2-c2ccc3c(ccc4nc(-c5ccccc5)n(-c5ccccc5)c43)c21 ; CC1(C)c2ccc(N(c3ccccc3)c3ccc4oc5ccccc5c4c3)cc2-c2ccc3c(ccc4nc(-c5ccccc5)n(-c5ccccc5)c43)c21]

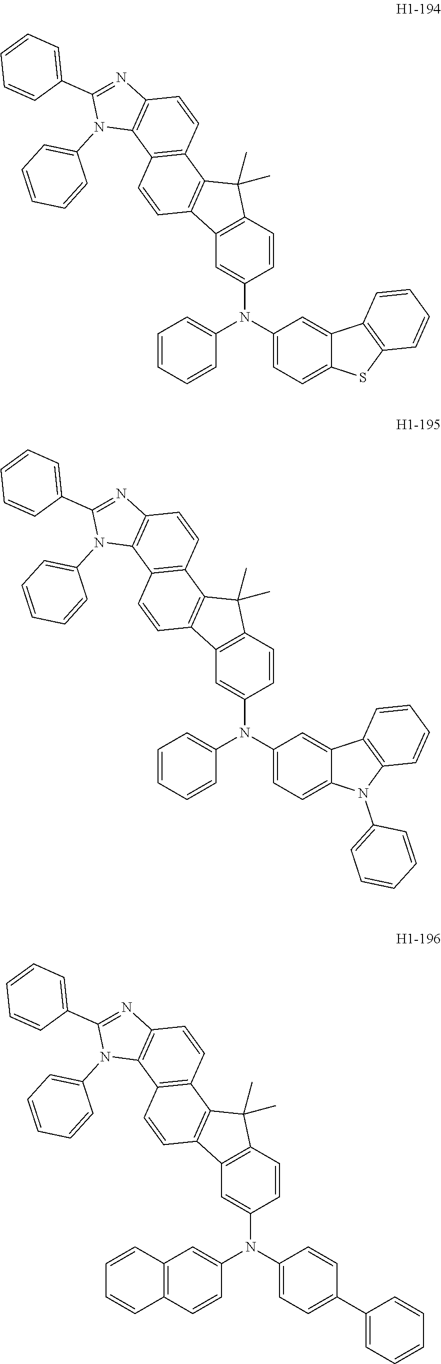 [SMILES: CC1(C)c2ccc(N(c3ccccc3)c3ccc4sc5ccccc5c4c3)cc2-c2ccc3c(ccc4nc(-c5ccccc5)n(-c5ccccc5)c43)c21 ; CC1(C)c2ccc(N(c3ccccc3)c3ccc4c(c3)c3ccccc3n4-c3ccccc3)cc2-c2ccc3c(ccc4nc(-c5ccccc5)n(-c5ccccc5)c43)c21 ; CC1(C)c2ccc(N(c3ccc(-c4ccccc4)cc3)c3ccc4ccccc4c3)cc2-c2ccc3c(ccc4nc(-c5ccccc5)n(-c5ccccc5)c43)c21]

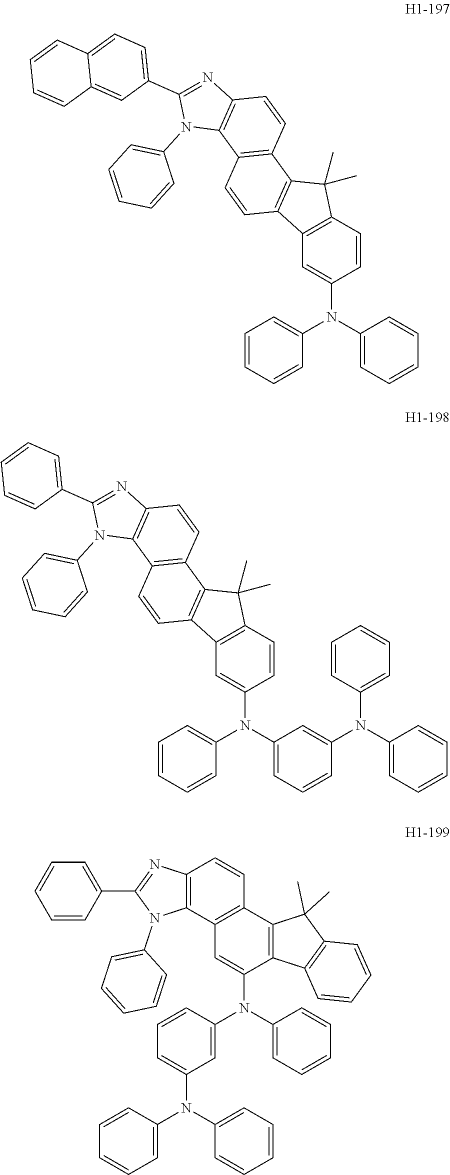 [SMILES: CC1(C)c2ccc(N(c3ccccc3)c3ccccc3)cc2-c2ccc3c(ccc4nc(-c5ccc6ccccc6c5)n(-c5ccccc5)c43)c21 ; CC1(C)c2ccc(N(c3ccccc3)c3cccc(N(c4ccccc4)c4ccccc4)c3)cc2-c2ccc3c(ccc4nc(-c5ccccc5)n(-c5ccccc5)c43)c21 ; CC1(C)c2ccccc2-c2c(N(c3ccccc3)c3cccc(N(c4ccccc4)c4ccccc4)c3)cc3c(ccc4nc(-c5ccccc5)n(-c5ccccc5)c43)c21]

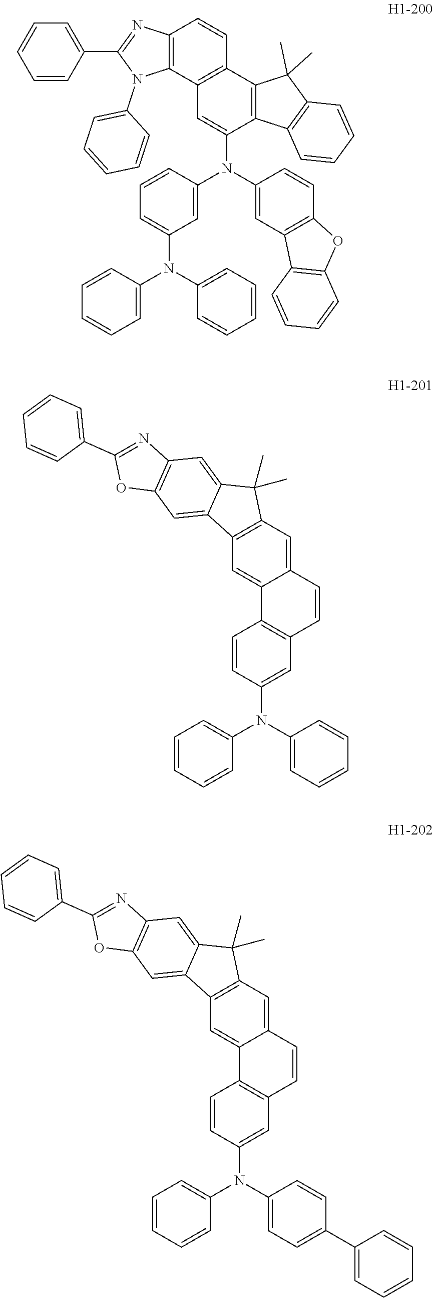 [SMILES: CC1(C)c2ccccc2-c2c(N(c3cccc(N(c4ccccc4)c4ccccc4)c3)c3ccc4oc5ccccc5c4c3)cc3c(ccc4nc(-c5ccccc5)n(-c5ccccc5)c43)c21 ; CC1(C)c2cc3ccc4cc(N(c5ccccc5)c5ccccc5)ccc4c3cc2-c2cc3oc(-c4ccccc4)nc3cc21 ; CC1(C)c2cc3ccc4cc(N(c5ccccc5)c5ccc(-c6ccccc6)cc5)ccc4c3cc2-c2cc3oc(-c4ccccc4)nc3cc21]

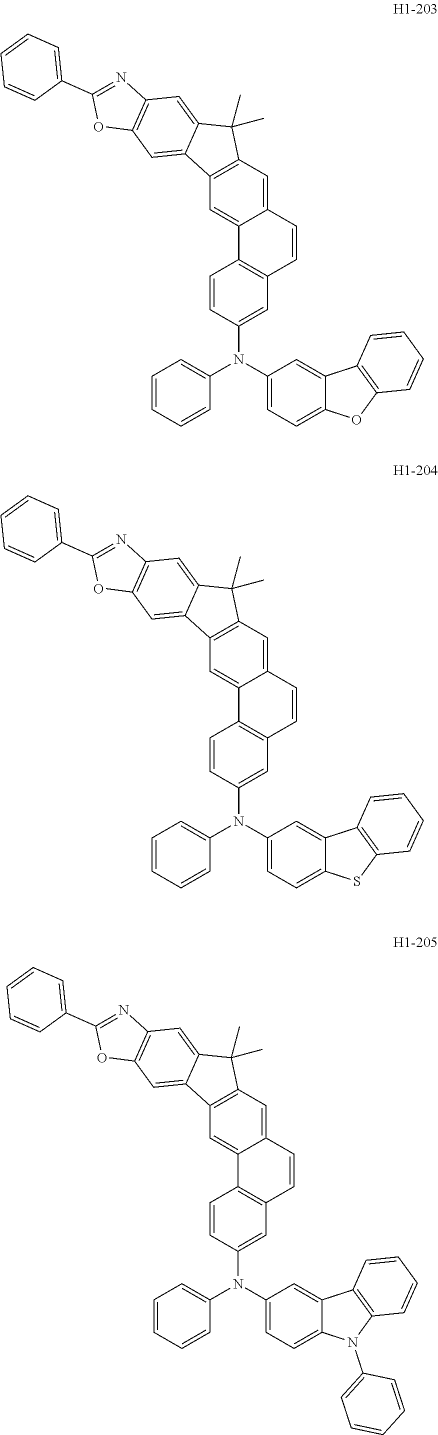 [SMILES: CC1(C)c2cc3ccc4cc(N(c5ccccc5)c5ccc6oc7ccccc7c6c5)ccc4c3cc2-c2cc3oc(-c4ccccc4)nc3cc21 ; CC1(C)c2cc3ccc4cc(N(c5ccccc5)c5ccc6sc7ccccc7c6c5)ccc4c3cc2-c2cc3oc(-c4ccccc4)nc3cc21 ; CC1(C)c2cc3ccc4cc(N(c5ccccc5)c5ccc6c(c5)c5ccccc5n6-c5ccccc5)ccc4c3cc2-c2cc3oc(-c4ccccc4)nc3cc21]

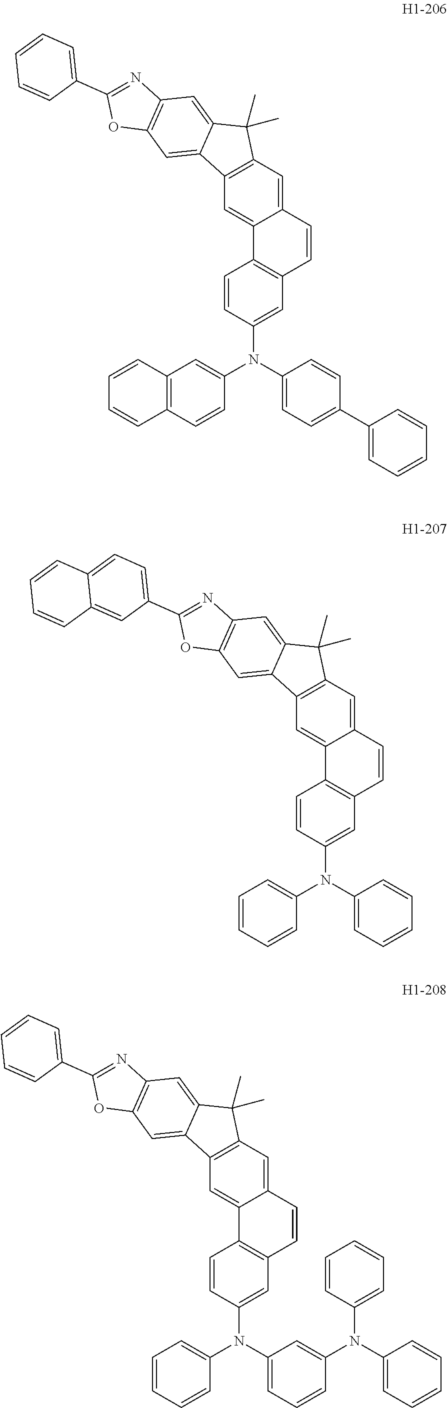 [SMILES: CC1(C)c2cc3ccc4cc(N(c5ccc(-c6ccccc6)cc5)c5ccc6ccccc6c5)ccc4c3cc2-c2cc3oc(-c4ccccc4)nc3cc21 ; CC1(C)c2cc3ccc4cc(N(c5ccccc5)c5ccccc5)ccc4c3cc2-c2cc3oc(-c4ccc5ccccc5c4)nc3cc21 ; CC1(C)c2cc3ccc4cc(N(c5ccccc5)c5cccc(N(c6ccccc6)c6ccccc6)c5)ccc4c3cc2-c2cc3oc(-c4ccccc4)nc3cc21]

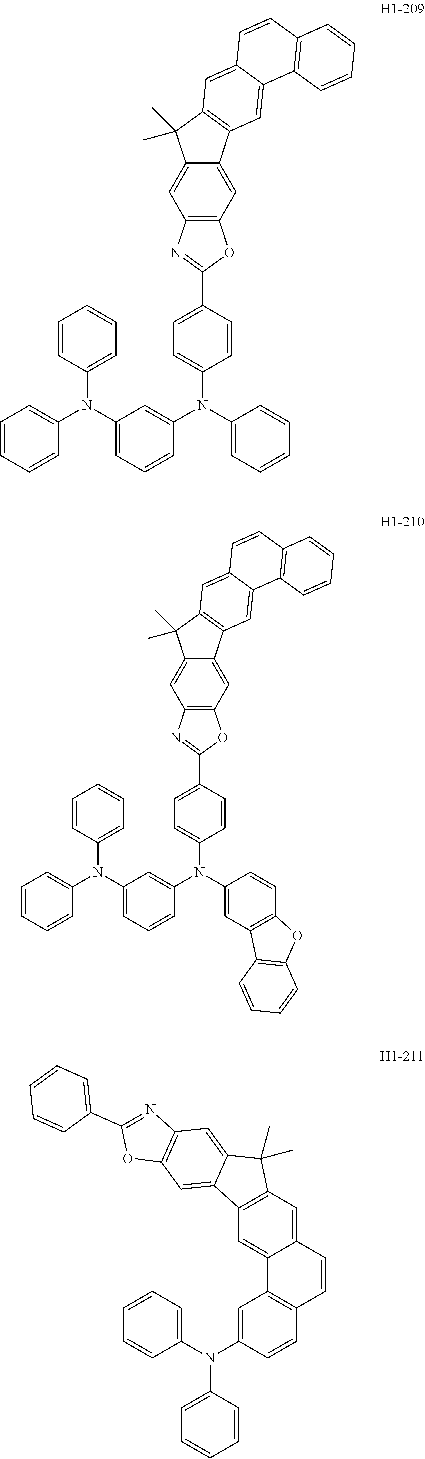 [SMILES: CC1(C)c2cc3ccc4ccccc4c3cc2-c2cc3oc(-c4ccc(N(c5ccccc5)c5cccc(N(c6ccccc6)c6ccccc6)c5)cc4)nc3cc21 ; CC1(C)c2cc3ccc4ccccc4c3cc2-c2cc3oc(-c4ccc(N(c5cccc(N(c6ccccc6)c6ccccc6)c5)c5ccc6oc7ccccc7c6c5)cc4)nc3cc21 ; CC1(C)c2cc3ccc4ccc(N(c5ccccc5)c5ccccc5)cc4c3cc2-c2cc3oc(-c4ccccc4)nc3cc21]

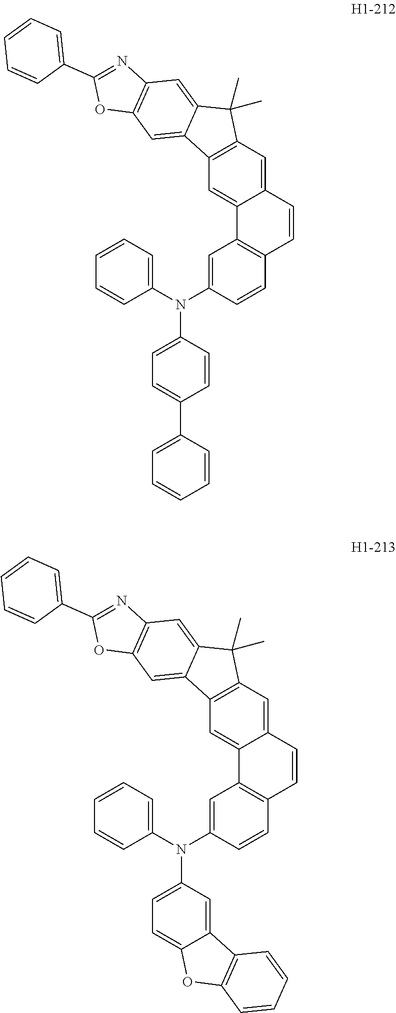 [SMILES: CC1(C)c2cc3ccc4ccc(N(c5ccccc5)c5ccc(-c6ccccc6)cc5)cc4c3cc2-c2cc3oc(-c4ccccc4)nc3cc21 ; CC1(C)c2cc3ccc4ccc(N(c5ccccc5)c5ccc6oc7ccccc7c6c5)cc4c3cc2-c2cc3oc(-c4ccccc4)nc3cc21]

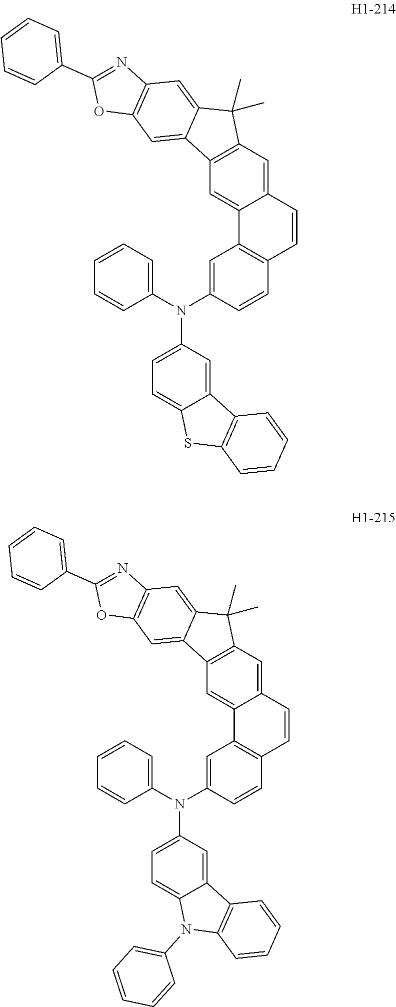 [SMILES: CC1(C)c2cc3ccc4ccc(N(c5ccccc5)c5ccc6sc7ccccc7c6c5)cc4c3cc2-c2cc3oc(-c4ccccc4)nc3cc21 ; CC1(C)c2cc3ccc4ccc(N(c5ccccc5)c5ccc6c(c5)c5ccccc5n6-c5ccccc5)cc4c3cc2-c2cc3oc(-c4ccccc4)nc3cc21]

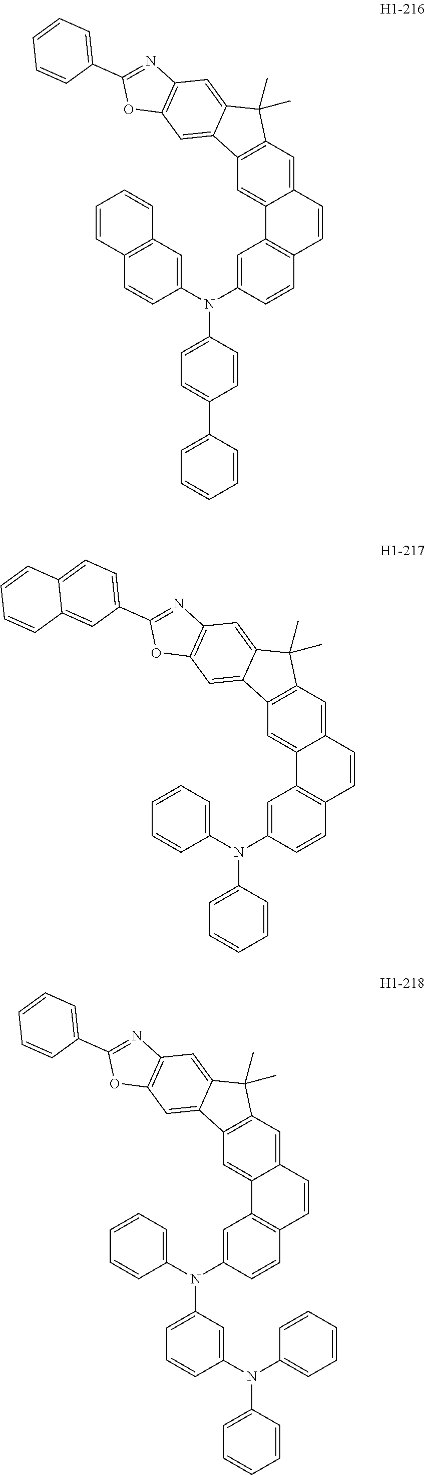 [SMILES: CC1(C)c2cc3ccc4ccc(N(c5ccc(-c6ccccc6)cc5)c5ccc6ccccc6c5)cc4c3cc2-c2cc3oc(-c4ccccc4)nc3cc21 ; CC1(C)c2cc3ccc4ccc(N(c5ccccc5)c5ccccc5)cc4c3cc2-c2cc3oc(-c4ccc5ccccc5c4)nc3cc21 ; CC1(C)c2cc3ccc4ccc(N(c5ccccc5)c5cccc(N(c6ccccc6)c6ccccc6)c5)cc4c3cc2-c2cc3oc(-c4ccccc4)nc3cc21]

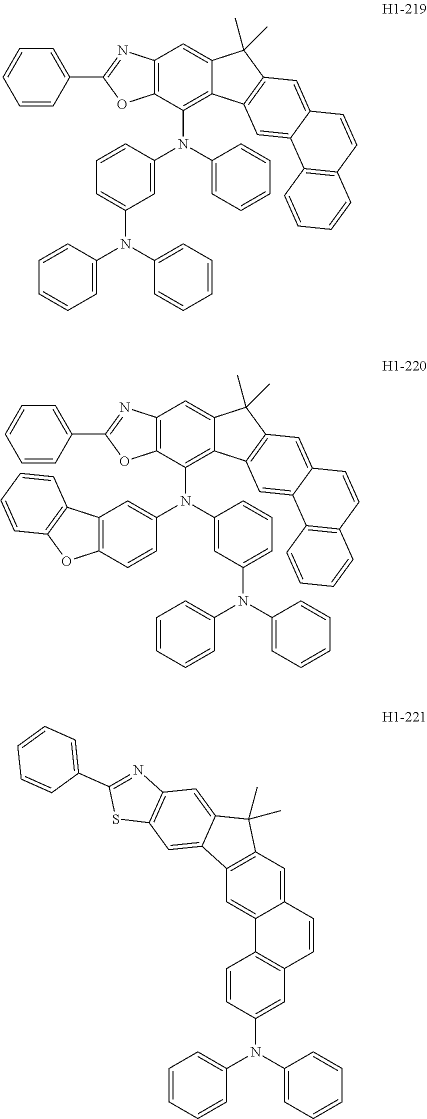 [SMILES: CC1(C)c2cc3ccc4ccccc4c3cc2-c2c1cc1nc(-c3ccccc3)oc1c2N(c1ccccc1)c1cccc(N(c2ccccc2)c2ccccc2)c1 ; CC1(C)c2cc3ccc4ccccc4c3cc2-c2c1cc1nc(-c3ccccc3)oc1c2N(c1cccc(N(c2ccccc2)c2ccccc2)c1)c1ccc2oc3ccccc3c2c1 ; CC1(C)c2cc3ccc4cc(N(c5ccccc5)c5ccccc5)ccc4c3cc2-c2cc3sc(-c4ccccc4)nc3cc21]

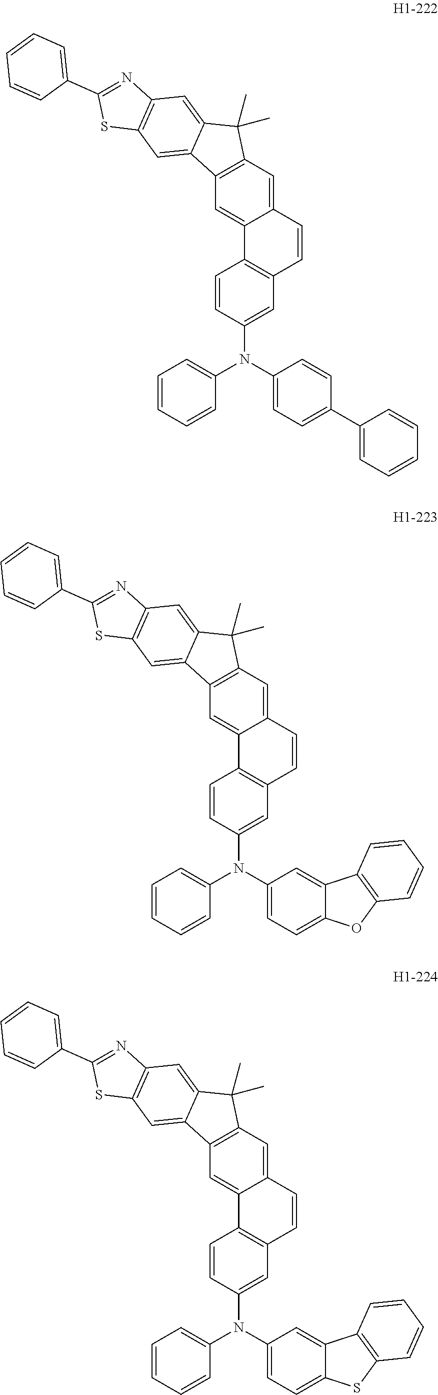 [SMILES: CC1(C)c2cc3ccc4cc(N(c5ccccc5)c5ccc(-c6ccccc6)cc5)ccc4c3cc2-c2cc3sc(-c4ccccc4)nc3cc21 ; CC1(C)c2cc3ccc4cc(N(c5ccccc5)c5ccc6oc7ccccc7c6c5)ccc4c3cc2-c2cc3sc(-c4ccccc4)nc3cc21 ; CC1(C)c2cc3ccc4cc(N(c5ccccc5)c5ccc6sc7ccccc7c6c5)ccc4c3cc2-c2cc3sc(-c4ccccc4)nc3cc21]

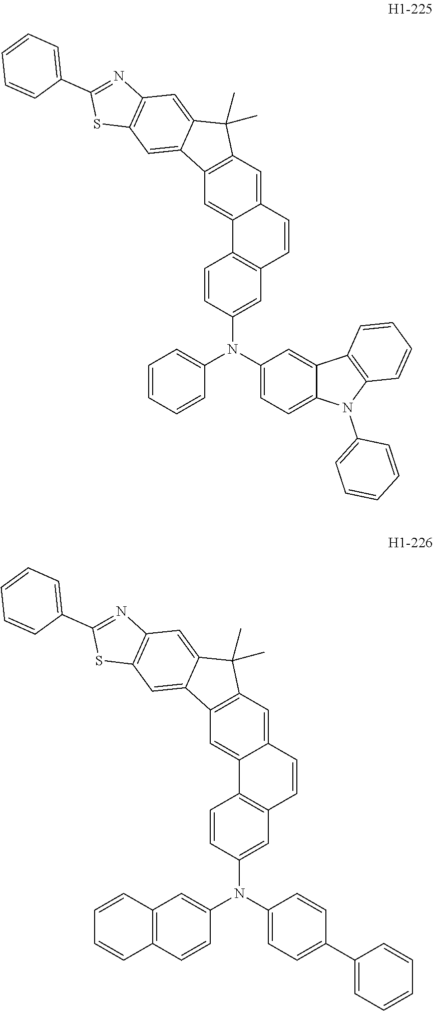 [SMILES: CC1(C)c2cc3ccc4cc(N(c5ccccc5)c5ccc6c(c5)c5ccccc5n6-c5ccccc5)ccc4c3cc2-c2cc3sc(-c4ccccc4)nc3cc21 ; CC1(C)c2cc3ccc4cc(N(c5ccc(-c6ccccc6)cc5)c5ccc6ccccc6c5)ccc4c3cc2-c2cc3sc(-c4ccccc4)nc3cc21]

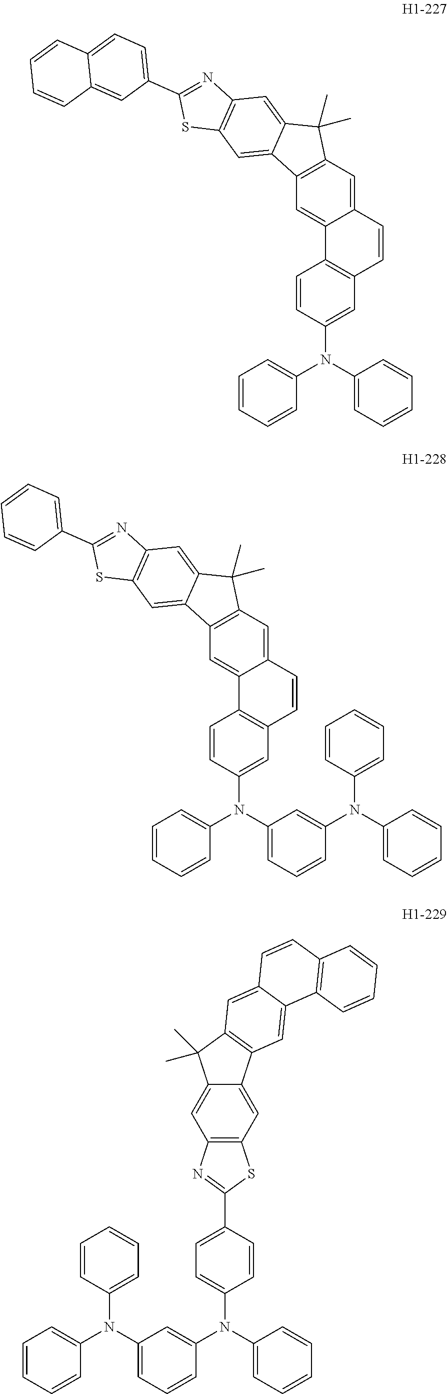 [SMILES: CC1(C)c2cc3ccc4cc(N(c5ccccc5)c5ccccc5)ccc4c3cc2-c2cc3sc(-c4ccc5ccccc5c4)nc3cc21 ; CC1(C)c2cc3ccc4cc(N(c5ccccc5)c5cccc(N(c6ccccc6)c6ccccc6)c5)ccc4c3cc2-c2cc3sc(-c4ccccc4)nc3cc21 ; CC1(C)c2cc3ccc4ccccc4c3cc2-c2cc3sc(-c4ccc(N(c5ccccc5)c5cccc(N(c6ccccc6)c6ccccc6)c5)cc4)nc3cc21]

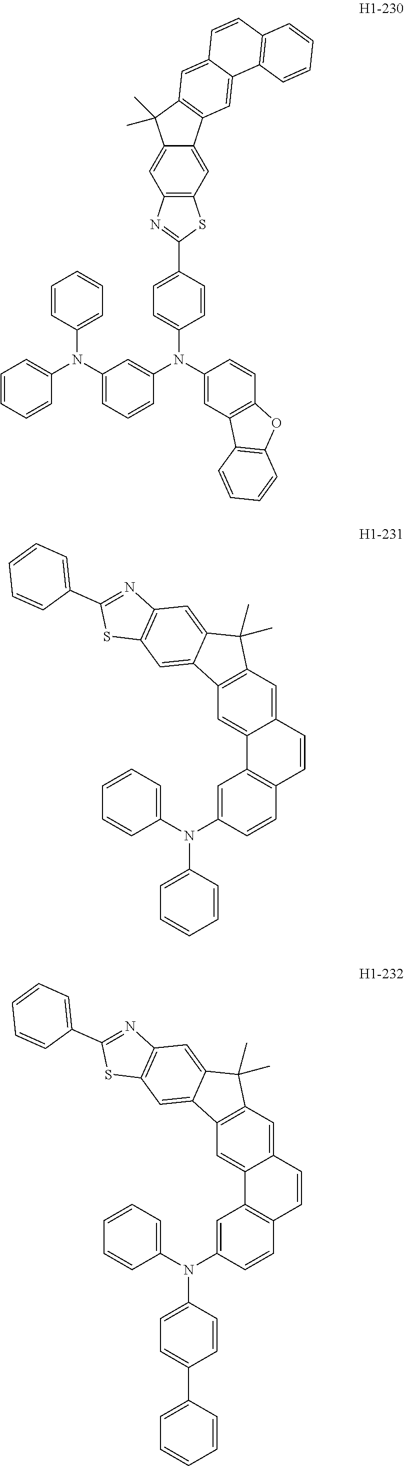 [SMILES: CC1(C)c2cc3ccc4ccccc4c3cc2-c2cc3sc(-c4ccc(N(c5cccc(N(c6ccccc6)c6ccccc6)c5)c5ccc6oc7ccccc7c6c5)cc4)nc3cc21 ; CC1(C)c2cc3ccc4ccc(N(c5ccccc5)c5ccccc5)cc4c3cc2-c2cc3sc(-c4ccccc4)nc3cc21 ; CC1(C)c2cc3ccc4ccc(N(c5ccccc5)c5ccc(-c6ccccc6)cc5)cc4c3cc2-c2cc3sc(-c4ccccc4)nc3cc21]

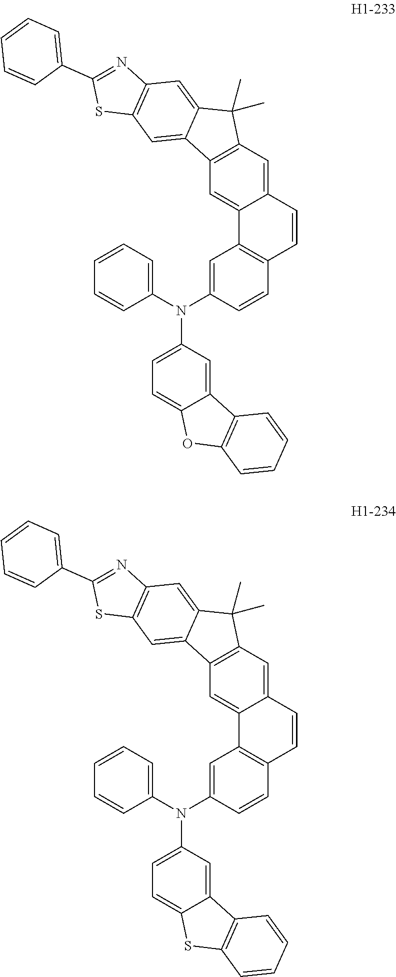 [SMILES: CC1(C)c2cc3ccc4ccc(N(c5ccccc5)c5ccc6oc7ccccc7c6c5)cc4c3cc2-c2cc3sc(-c4ccccc4)nc3cc21 ; CC1(C)c2cc3ccc4ccc(N(c5ccccc5)c5ccc6sc7ccccc7c6c5)cc4c3cc2-c2cc3sc(-c4ccccc4)nc3cc21]

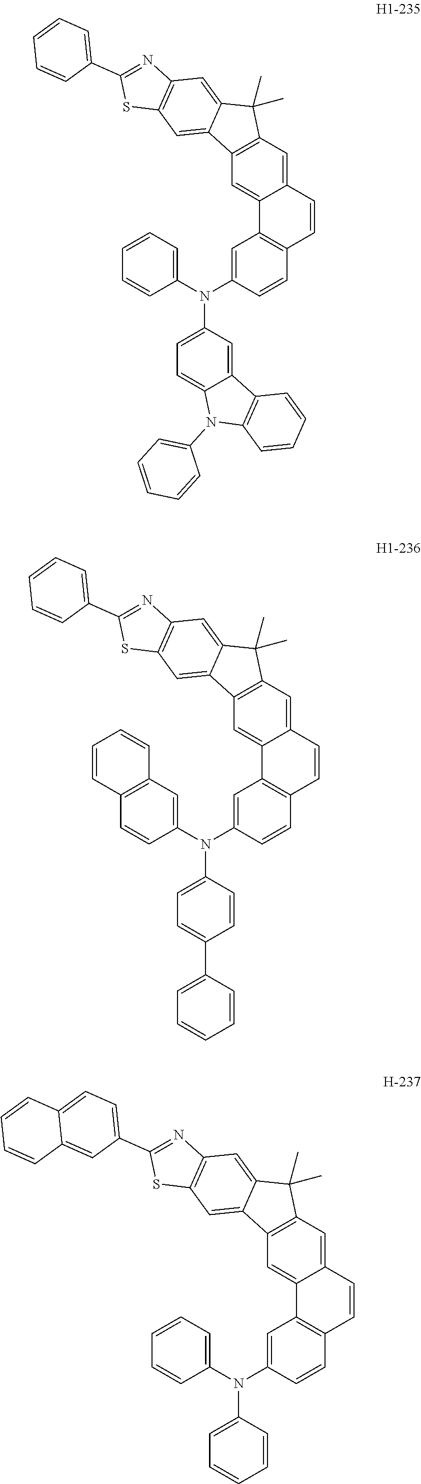 [SMILES: CC1(C)c2cc3ccc4ccc(N(c5ccccc5)c5ccc6c(c5)c5ccccc5n6-c5ccccc5)cc4c3cc2-c2cc3sc(-c4ccccc4)nc3cc21 ; CC1(C)c2cc3ccc4ccc(N(c5ccc(-c6ccccc6)cc5)c5ccc6ccccc6c5)cc4c3cc2-c2cc3sc(-c4ccccc4)nc3cc21 ; CC1(C)c2cc3ccc4ccc(N(c5ccccc5)c5ccccc5)cc4c3cc2-c2cc3sc(-c4ccc5ccccc5c4)nc3cc21]

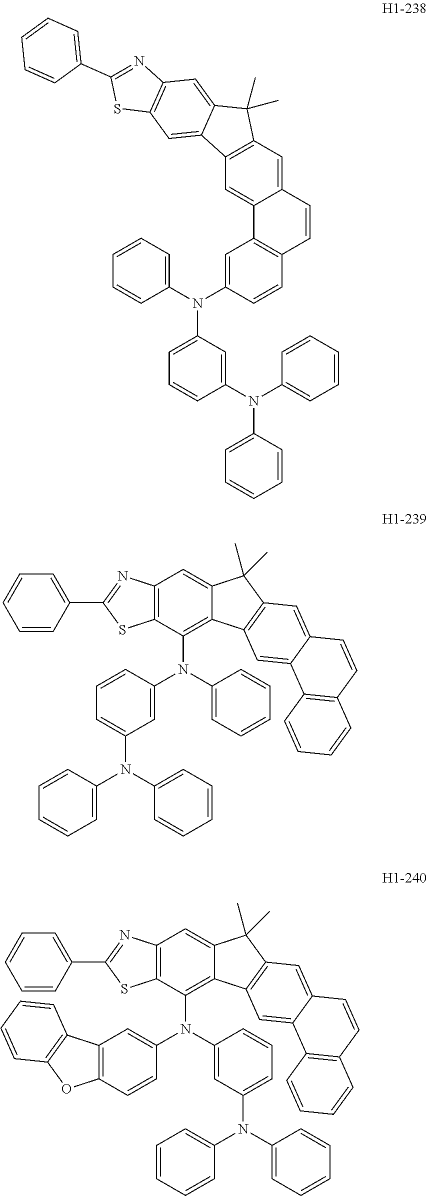 [SMILES: CC1(C)c2cc3ccc4ccc(N(c5ccccc5)c5cccc(N(c6ccccc6)c6ccccc6)c5)cc4c3cc2-c2cc3sc(-c4ccccc4)nc3cc21 ; CC1(C)c2cc3ccc4ccccc4c3cc2-c2c1cc1nc(-c3ccccc3)sc1c2N(c1ccccc1)c1cccc(N(c2ccccc2)c2ccccc2)c1 ; CC1(C)c2cc3ccc4ccccc4c3cc2-c2c1cc1nc(-c3ccccc3)sc1c2N(c1cccc(N(c2ccccc2)c2ccccc2)c1)c1ccc2oc3ccccc3c2c1]

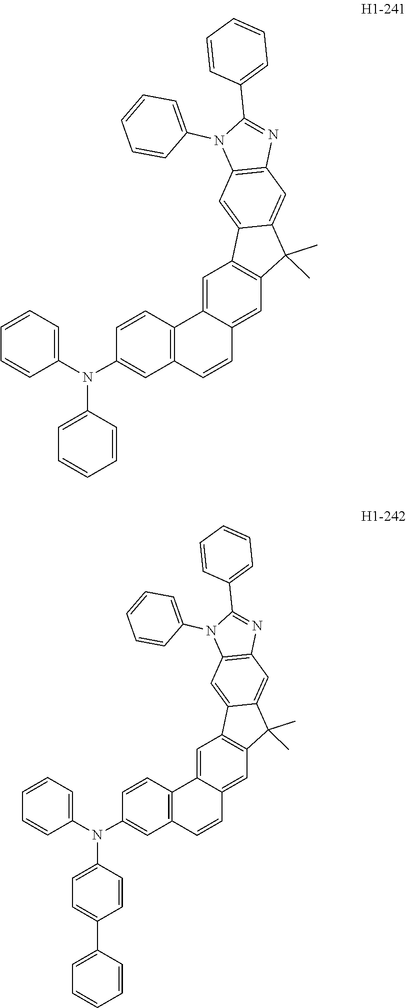 [SMILES: CC1(C)c2cc3ccc4cc(N(c5ccccc5)c5ccccc5)ccc4c3cc2-c2cc3c(cc21)nc(-c1ccccc1)n3-c1ccccc1 ; CC1(C)c2cc3ccc4cc(N(c5ccccc5)c5ccc(-c6ccccc6)cc5)ccc4c3cc2-c2cc3c(cc21)nc(-c1ccccc1)n3-c1ccccc1]

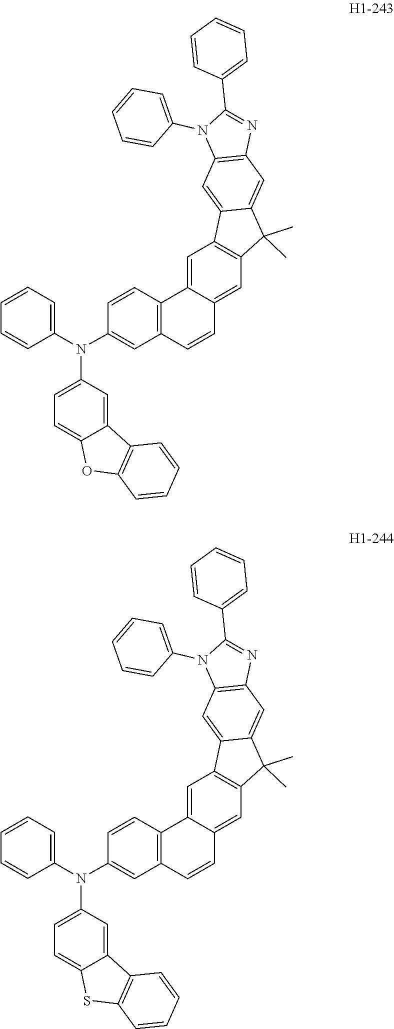 [SMILES: CC1(C)c2cc3ccc4cc(N(c5ccccc5)c5ccc6oc7ccccc7c6c5)ccc4c3cc2-c2cc3c(cc21)nc(-c1ccccc1)n3-c1ccccc1 ; CC1(C)c2cc3ccc4cc(N(c5ccccc5)c5ccc6sc7ccccc7c6c5)ccc4c3cc2-c2cc3c(cc21)nc(-c1ccccc1)n3-c1ccccc1]

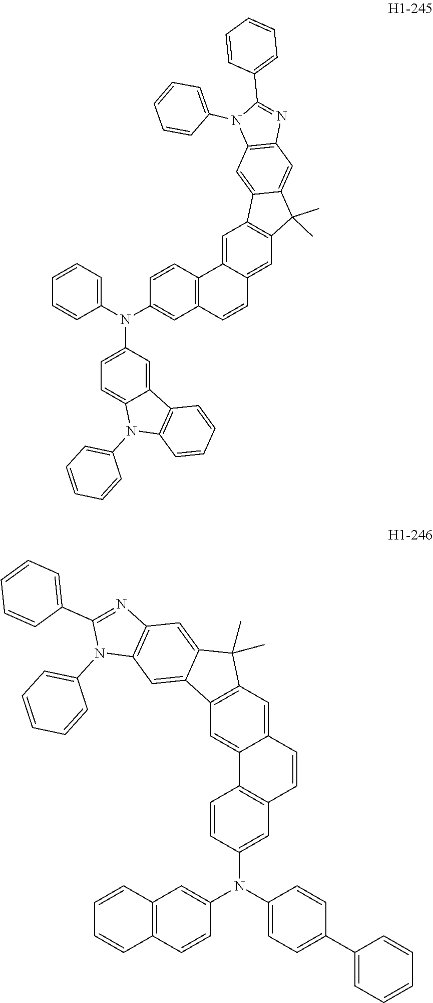 [SMILES: CC1(C)c2cc3ccc4cc(N(c5ccccc5)c5ccc6c(c5)c5ccccc5n6-c5ccccc5)ccc4c3cc2-c2cc3c(cc21)nc(-c1ccccc1)n3-c1ccccc1 ; CC1(C)c2cc3ccc4cc(N(c5ccc(-c6ccccc6)cc5)c5ccc6ccccc6c5)ccc4c3cc2-c2cc3c(cc21)nc(-c1ccccc1)n3-c1ccccc1]

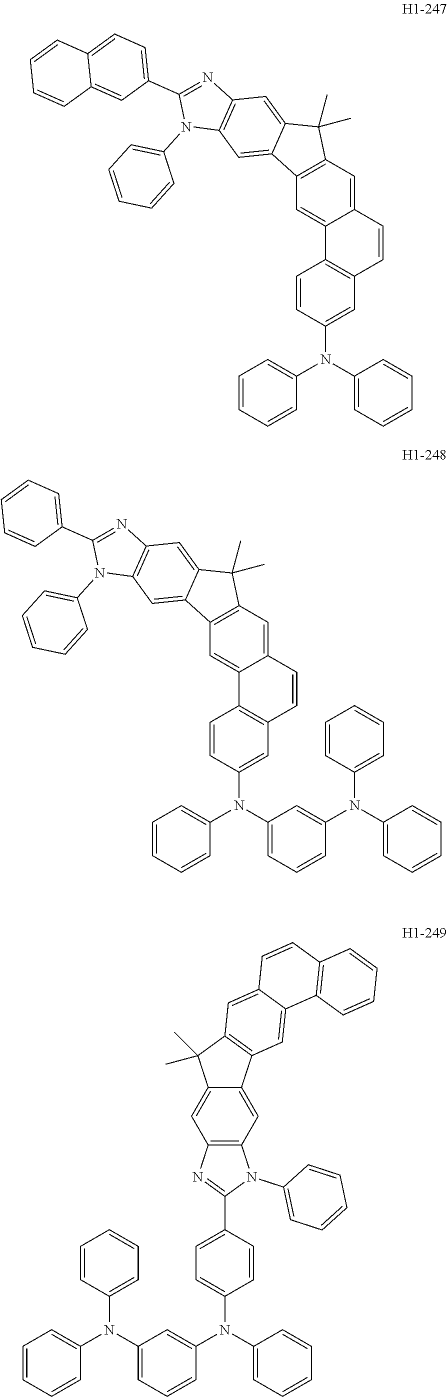 [SMILES: CC1(C)c2cc3ccc4cc(N(c5ccccc5)c5ccccc5)ccc4c3cc2-c2cc3c(cc21)nc(-c1ccc2ccccc2c1)n3-c1ccccc1 ; CC1(C)c2cc3ccc4cc(N(c5ccccc5)c5cccc(N(c6ccccc6)c6ccccc6)c5)ccc4c3cc2-c2cc3c(cc21)nc(-c1ccccc1)n3-c1ccccc1 ; CC1(C)c2cc3ccc4ccccc4c3cc2-c2cc3c(cc21)nc(-c1ccc(N(c2ccccc2)c2cccc(N(c4ccccc4)c4ccccc4)c2)cc1)n3-c1ccccc1]

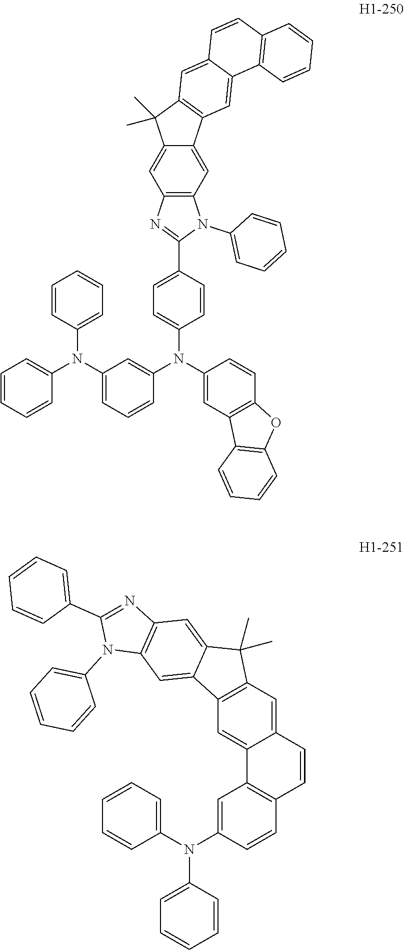 [SMILES: CC1(C)c2cc3ccc4ccccc4c3cc2-c2cc3c(cc21)nc(-c1ccc(N(c2cccc(N(c4ccccc4)c4ccccc4)c2)c2ccc4oc5ccccc5c4c2)cc1)n3-c1ccccc1 ; CC1(C)c2cc3ccc4ccc(N(c5ccccc5)c5ccccc5)cc4c3cc2-c2cc3c(cc21)nc(-c1ccccc1)n3-c1ccccc1]

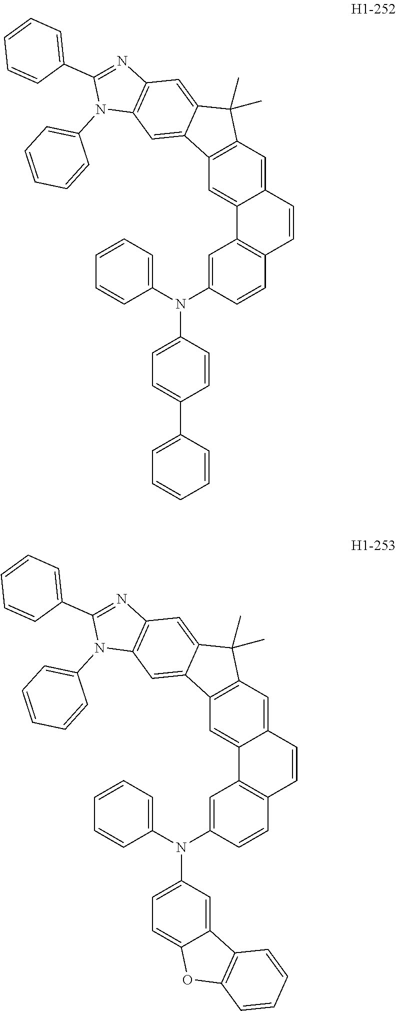 [SMILES: CC1(C)c2cc3ccc4ccc(N(c5ccccc5)c5ccc(-c6ccccc6)cc5)cc4c3cc2-c2cc3c(cc21)nc(-c1ccccc1)n3-c1ccccc1 ; CC1(C)c2cc3ccc4ccc(N(c5ccccc5)c5ccc6oc7ccccc7c6c5)cc4c3cc2-c2cc3c(cc21)nc(-c1ccccc1)n3-c1ccccc1]

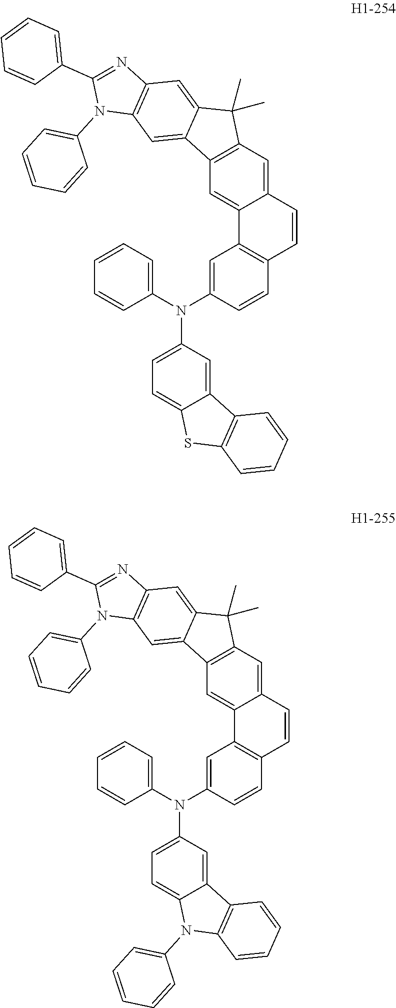 [SMILES: CC1(C)c2cc3ccc4ccc(N(c5ccccc5)c5ccc6sc7ccccc7c6c5)cc4c3cc2-c2cc3c(cc21)nc(-c1ccccc1)n3-c1ccccc1 ; CC1(C)c2cc3ccc4ccc(N(c5ccccc5)c5ccc6c(c5)c5ccccc5n6-c5ccccc5)cc4c3cc2-c2cc3c(cc21)nc(-c1ccccc1)n3-c1ccccc1]

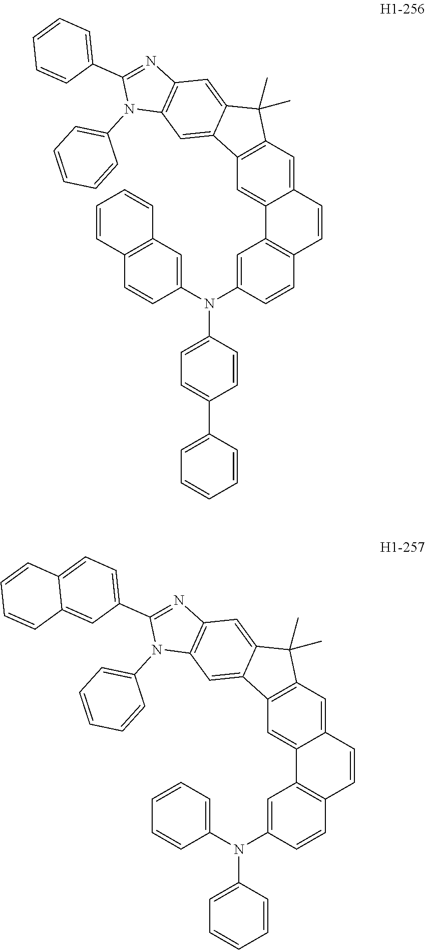 [SMILES: CC1(C)c2cc3ccc4ccc(N(c5ccc(-c6ccccc6)cc5)c5ccc6ccccc6c5)cc4c3cc2-c2cc3c(cc21)nc(-c1ccccc1)n3-c1ccccc1 ; CC1(C)c2cc3ccc4ccc(N(c5ccccc5)c5ccccc5)cc4c3cc2-c2cc3c(cc21)nc(-c1ccc2ccccc2c1)n3-c1ccccc1]

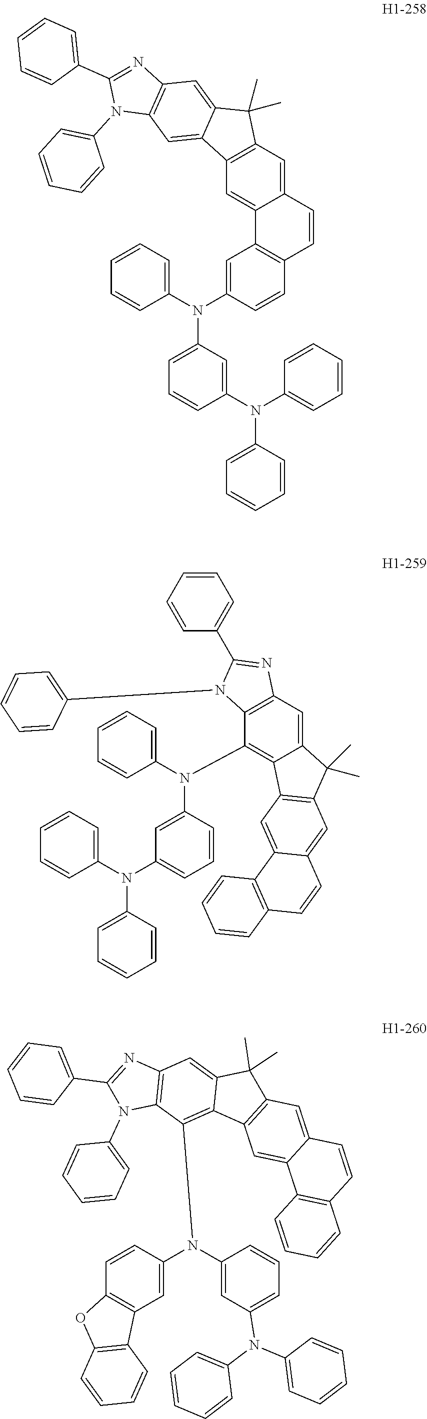 [SMILES: CC1(C)c2cc3ccc4ccc(N(c5ccccc5)c5cccc(N(c6ccccc6)c6ccccc6)c5)cc4c3cc2-c2cc3c(cc21)nc(-c1ccccc1)n3-c1ccccc1 ; CC1(C)c2cc3ccc4ccccc4c3cc2-c2c1cc1nc(-c3ccccc3)n(-c3ccccc3)c1c2N(c1ccccc1)c1cccc(N(c2ccccc2)c2ccccc2)c1 ; CC1(C)c2cc3ccc4ccccc4c3cc2-c2c1cc1nc(-c3ccccc3)n(-c3ccccc3)c1c2N(c1cccc(N(c2ccccc2)c2ccccc2)c1)c1ccc2oc3ccccc3c2c1]

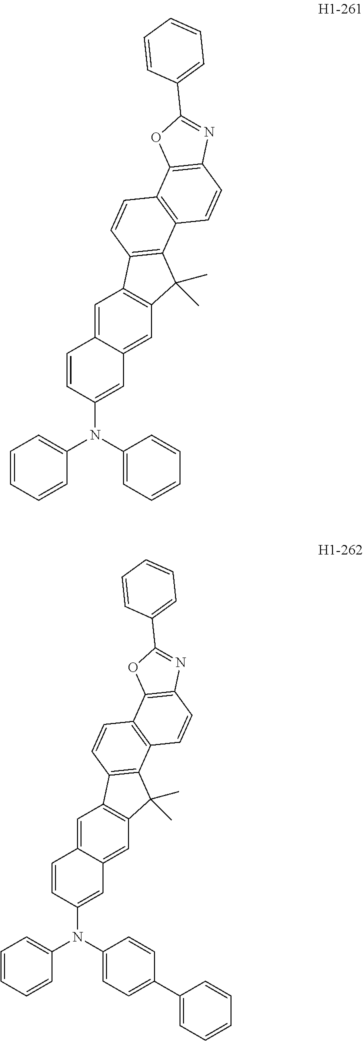 [SMILES: CC1(C)c2cc3cc(N(c4ccccc4)c4ccccc4)ccc3cc2-c2ccc3c(ccc4nc(-c5ccccc5)oc43)c21 ; CC1(C)c2cc3cc(N(c4ccccc4)c4ccc(-c5ccccc5)cc4)ccc3cc2-c2ccc3c(ccc4nc(-c5ccccc5)oc43)c21]

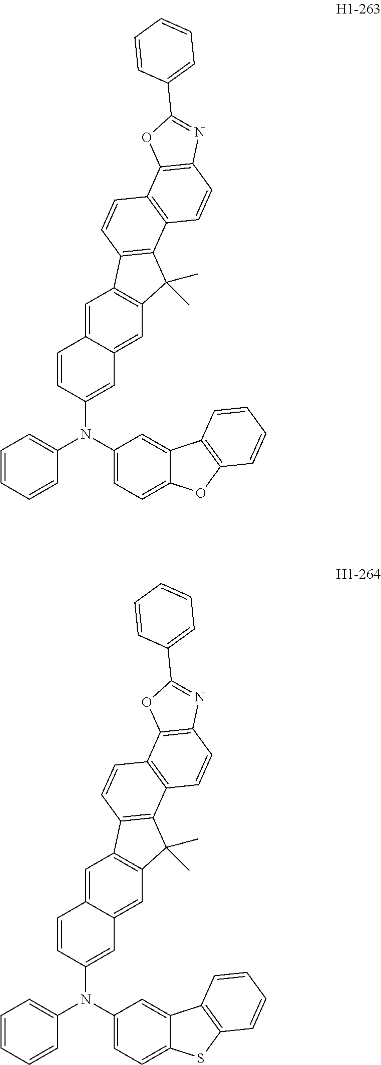 [SMILES: CC1(C)c2cc3cc(N(c4ccccc4)c4ccc5oc6ccccc6c5c4)ccc3cc2-c2ccc3c(ccc4nc(-c5ccccc5)oc43)c21 ; CC1(C)c2cc3cc(N(c4ccccc4)c4ccc5sc6ccccc6c5c4)ccc3cc2-c2ccc3c(ccc4nc(-c5ccccc5)oc43)c21]

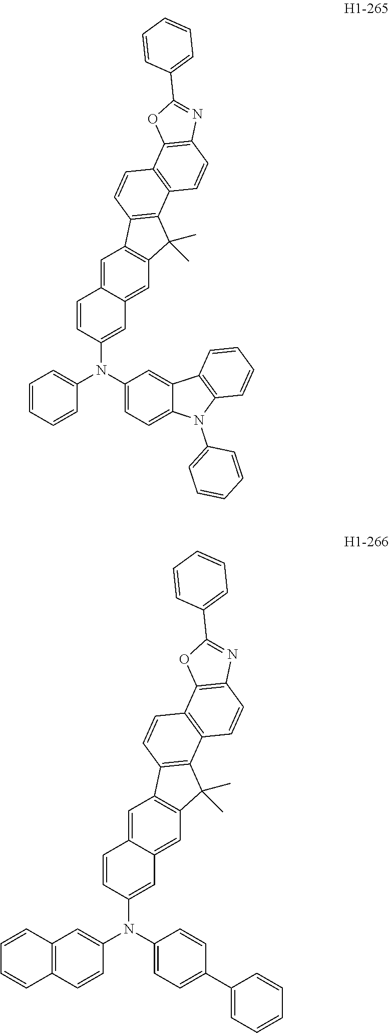 [SMILES: CC1(C)c2cc3cc(N(c4ccccc4)c4ccc5c(c4)c4ccccc4n5-c4ccccc4)ccc3cc2-c2ccc3c(ccc4nc(-c5ccccc5)oc43)c21 ; CC1(C)c2cc3cc(N(c4ccc(-c5ccccc5)cc4)c4ccc5ccccc5c4)ccc3cc2-c2ccc3c(ccc4nc(-c5ccccc5)oc43)c21]

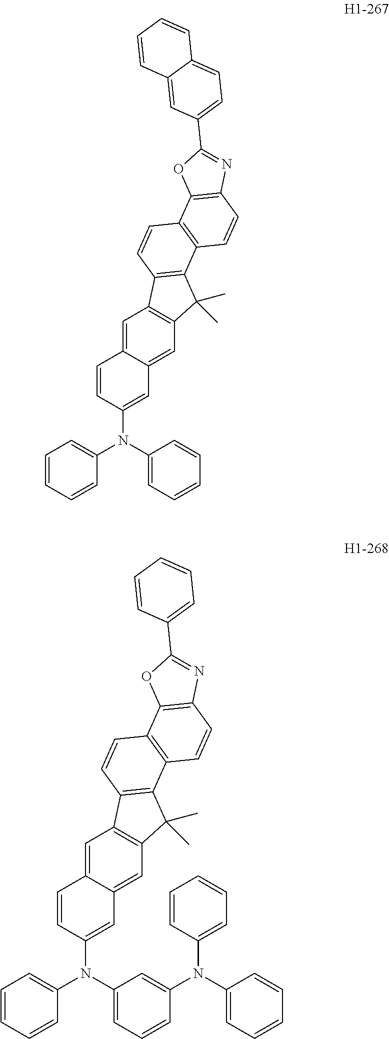 [SMILES: CC1(C)c2cc3cc(N(c4ccccc4)c4ccccc4)ccc3cc2-c2ccc3c(ccc4nc(-c5ccc6ccccc6c5)oc43)c21 ; CC1(C)c2cc3cc(N(c4ccccc4)c4cccc(N(c5ccccc5)c5ccccc5)c4)ccc3cc2-c2ccc3c(ccc4nc(-c5ccccc5)oc43)c21]

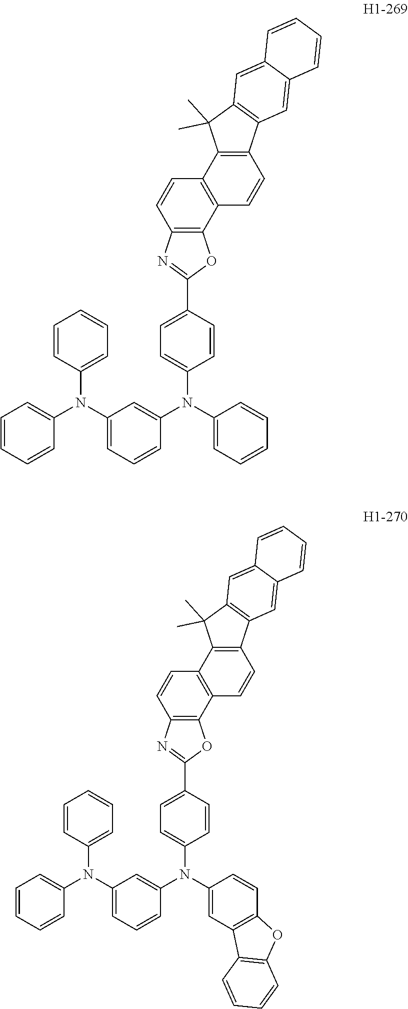 [SMILES: CC1(C)c2cc3ccccc3cc2-c2ccc3c(ccc4nc(-c5ccc(N(c6ccccc6)c6cccc(N(c7ccccc7)c7ccccc7)c6)cc5)oc43)c21 ; CC1(C)c2cc3ccccc3cc2-c2ccc3c(ccc4nc(-c5ccc(N(c6cccc(N(c7ccccc7)c7ccccc7)c6)c6ccc7oc8ccccc8c7c6)cc5)oc43)c21]

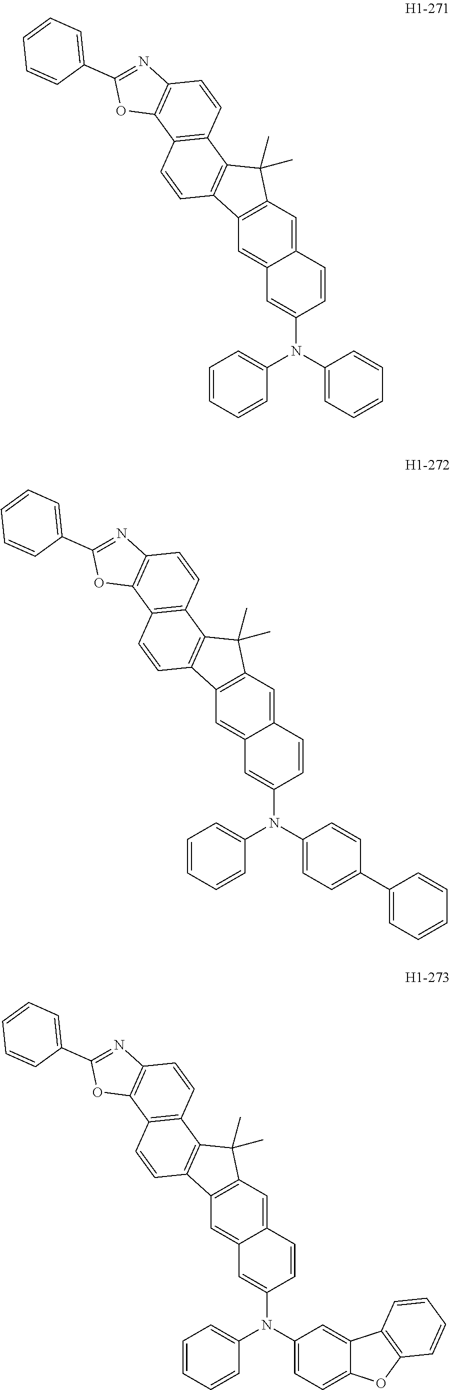 [SMILES: CC1(C)c2cc3ccc(N(c4ccccc4)c4ccccc4)cc3cc2-c2ccc3c(ccc4nc(-c5ccccc5)oc43)c21 ; CC1(C)c2cc3ccc(N(c4ccccc4)c4ccc(-c5ccccc5)cc4)cc3cc2-c2ccc3c(ccc4nc(-c5ccccc5)oc43)c21 ; CC1(C)c2cc3ccc(N(c4ccccc4)c4ccc5oc6ccccc6c5c4)cc3cc2-c2ccc3c(ccc4nc(-c5ccccc5)oc43)c21]

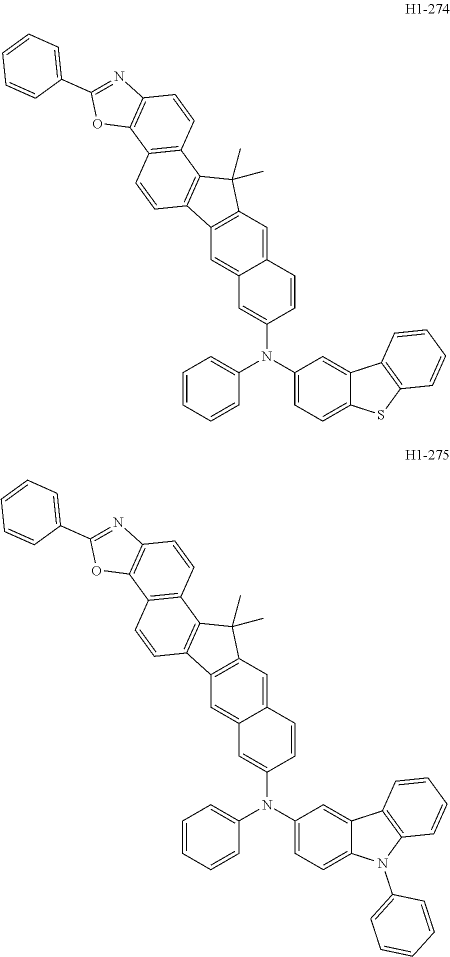 [SMILES: CC1(C)c2cc3ccc(N(c4ccccc4)c4ccc5sc6ccccc6c5c4)cc3cc2-c2ccc3c(ccc4nc(-c5ccccc5)oc43)c21 ; CC1(C)c2cc3ccc(N(c4ccccc4)c4ccc5c(c4)c4ccccc4n5-c4ccccc4)cc3cc2-c2ccc3c(ccc4nc(-c5ccccc5)oc43)c21]

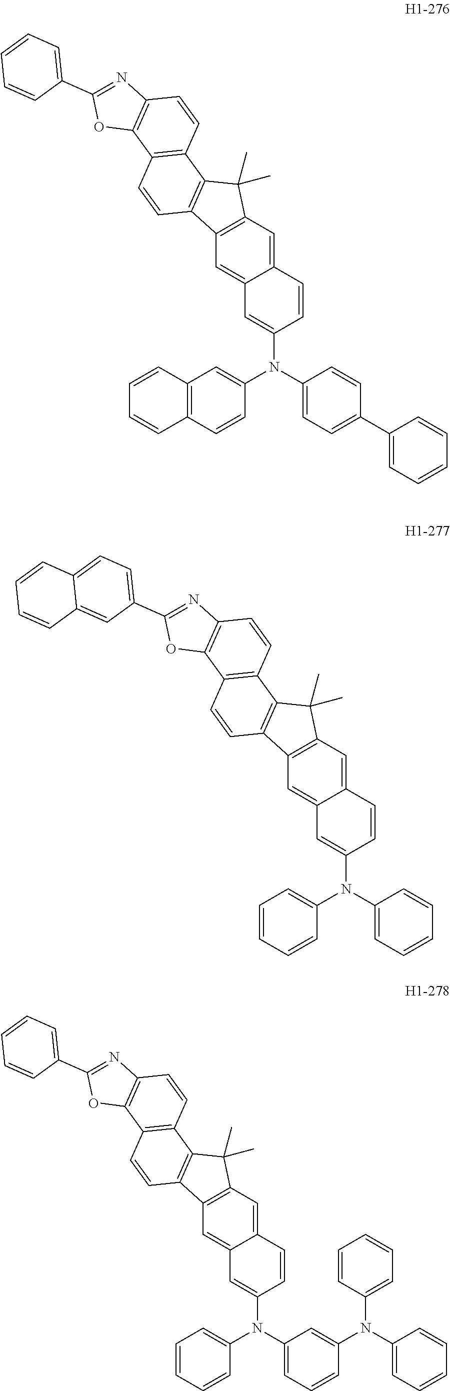 [SMILES: CC1(C)c2cc3ccc(N(c4ccc(-c5ccccc5)cc4)c4ccc5ccccc5c4)cc3cc2-c2ccc3c(ccc4nc(-c5ccccc5)oc43)c21 ; CC1(C)c2cc3ccc(N(c4ccccc4)c4ccccc4)cc3cc2-c2ccc3c(ccc4nc(-c5ccc6ccccc6c5)oc43)c21 ; CC1(C)c2cc3ccc(N(c4ccccc4)c4cccc(N(c5ccccc5)c5ccccc5)c4)cc3cc2-c2ccc3c(ccc4nc(-c5ccccc5)oc43)c21]

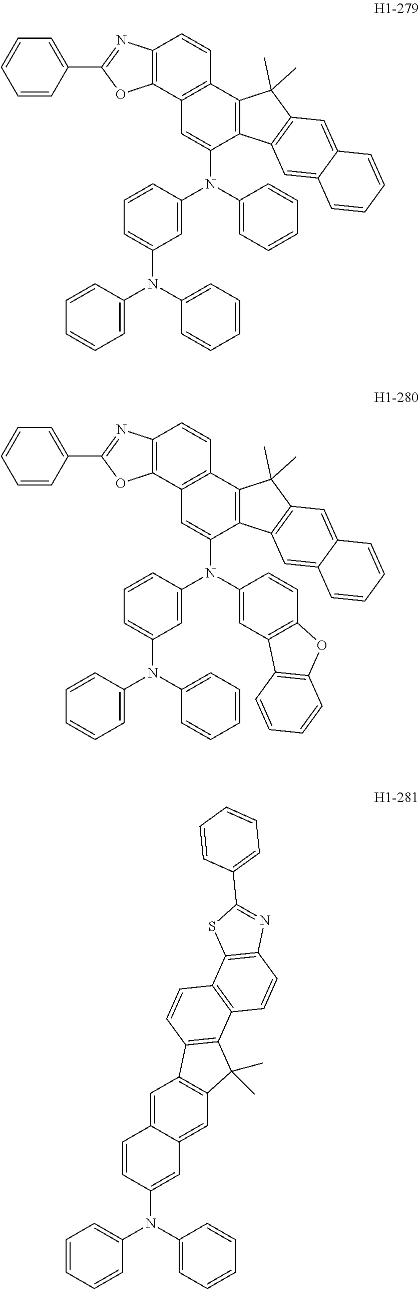 [SMILES: CC1(C)c2cc3ccccc3cc2-c2c(N(c3ccccc3)c3cccc(N(c4ccccc4)c4ccccc4)c3)cc3c(ccc4nc(-c5ccccc5)oc43)c21 ; CC1(C)c2cc3ccccc3cc2-c2c(N(c3cccc(N(c4ccccc4)c4ccccc4)c3)c3ccc4oc5ccccc5c4c3)cc3c(ccc4nc(-c5ccccc5)oc43)c21 ; CC1(C)c2cc3cc(N(c4ccccc4)c4ccccc4)ccc3cc2-c2ccc3c(ccc4nc(-c5ccccc5)sc43)c21]

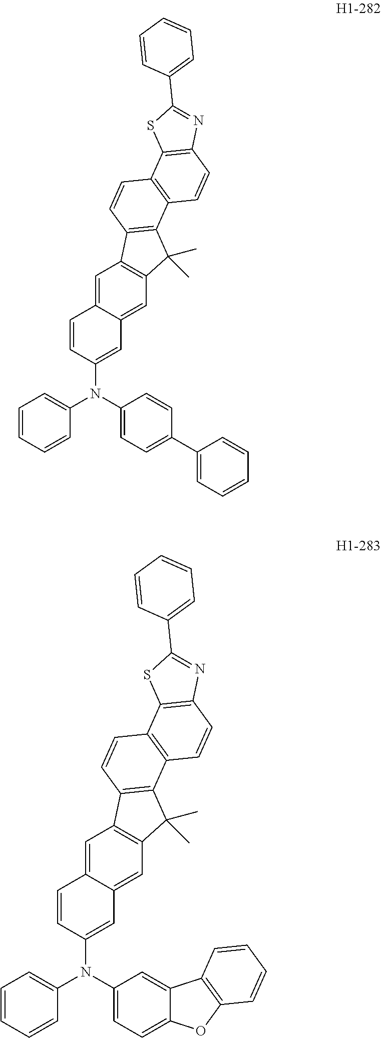 [SMILES: CC1(C)c2cc3cc(N(c4ccccc4)c4ccc(-c5ccccc5)cc4)ccc3cc2-c2ccc3c(ccc4nc(-c5ccccc5)sc43)c21 ; CC1(C)c2cc3cc(N(c4ccccc4)c4ccc5oc6ccccc6c5c4)ccc3cc2-c2ccc3c(ccc4nc(-c5ccccc5)sc43)c21]

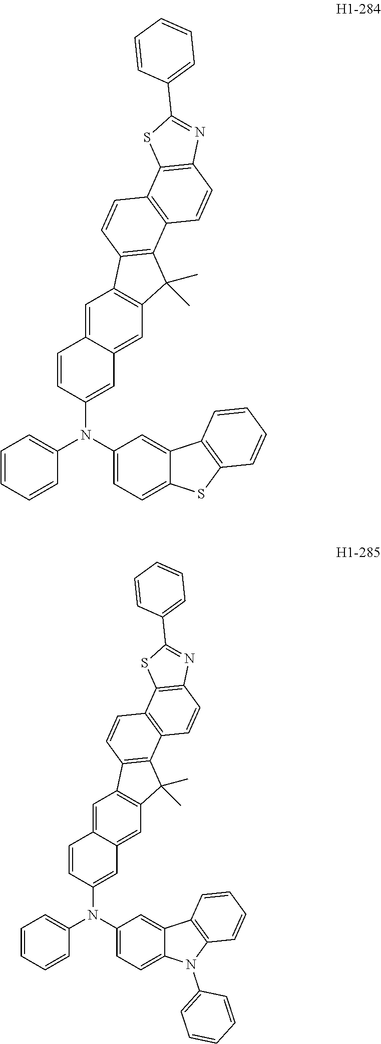 [SMILES: CC1(C)c2cc3cc(N(c4ccccc4)c4ccc5sc6ccccc6c5c4)ccc3cc2-c2ccc3c(ccc4nc(-c5ccccc5)sc43)c21 ; CC1(C)c2cc3cc(N(c4ccccc4)c4ccc5c(c4)c4ccccc4n5-c4ccccc4)ccc3cc2-c2ccc3c(ccc4nc(-c5ccccc5)sc43)c21]

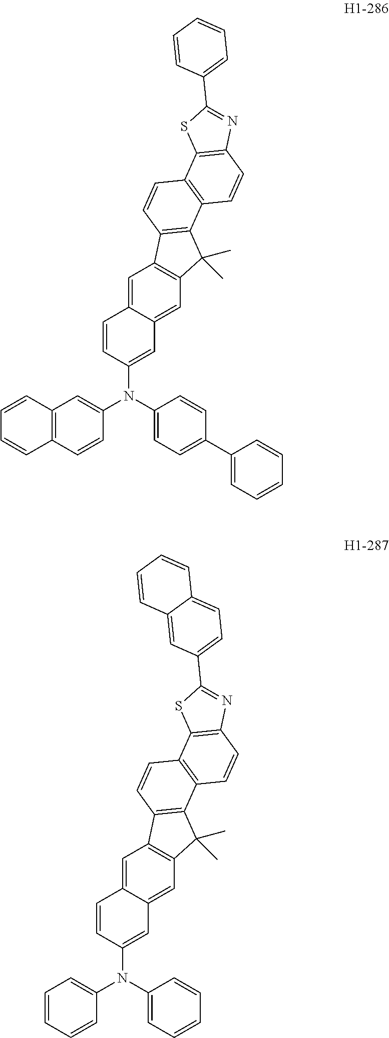 [SMILES: CC1(C)c2cc3cc(N(c4ccc(-c5ccccc5)cc4)c4ccc5ccccc5c4)ccc3cc2-c2ccc3c(ccc4nc(-c5ccccc5)sc43)c21 ; CC1(C)c2cc3cc(N(c4ccccc4)c4ccccc4)ccc3cc2-c2ccc3c(ccc4nc(-c5ccc6ccccc6c5)sc43)c21]

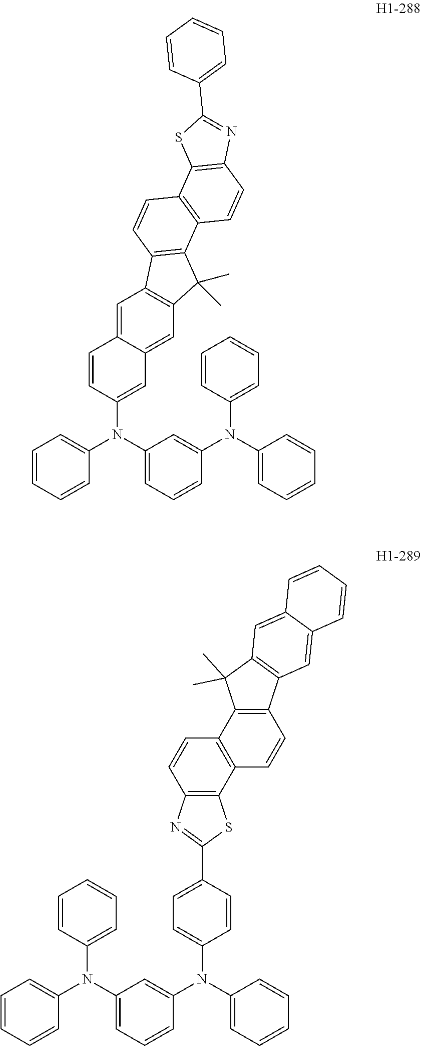 [SMILES: CC1(C)c2cc3cc(N(c4ccccc4)c4cccc(N(c5ccccc5)c5ccccc5)c4)ccc3cc2-c2ccc3c(ccc4nc(-c5ccccc5)sc43)c21 ; CC1(C)c2cc3ccccc3cc2-c2ccc3c(ccc4nc(-c5ccc(N(c6ccccc6)c6cccc(N(c7ccccc7)c7ccccc7)c6)cc5)sc43)c21]

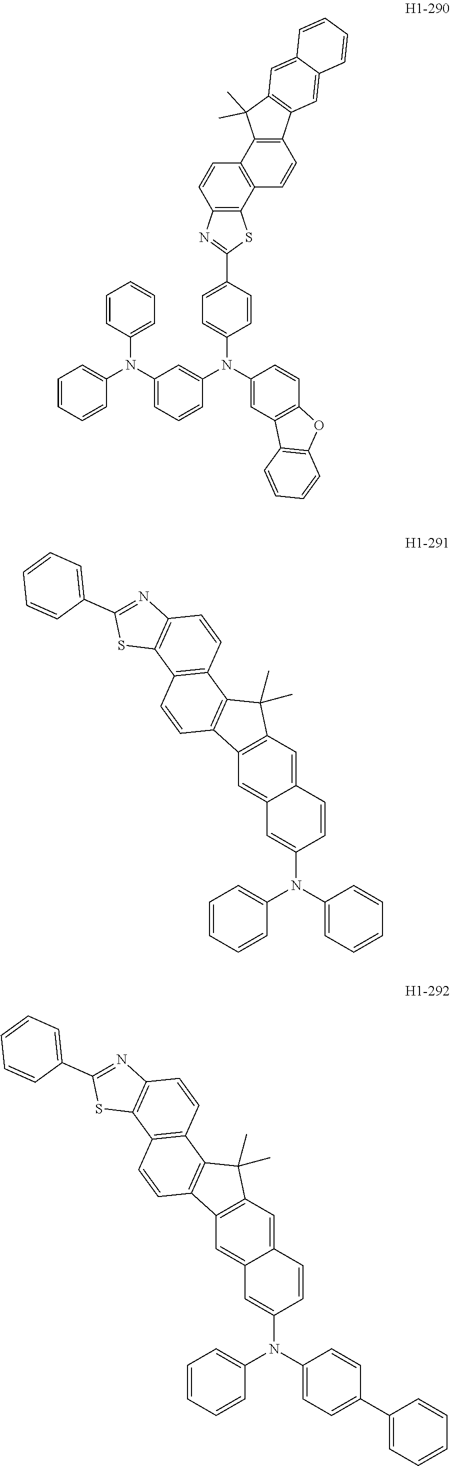 [SMILES: CC1(C)c2cc3ccccc3cc2-c2ccc3c(ccc4nc(-c5ccc(N(c6cccc(N(c7ccccc7)c7ccccc7)c6)c6ccc7oc8ccccc8c7c6)cc5)sc43)c21 ; CC1(C)c2cc3ccc(N(c4ccccc4)c4ccccc4)cc3cc2-c2ccc3c(ccc4nc(-c5ccccc5)sc43)c21 ; CC1(C)c2cc3ccc(N(c4ccccc4)c4ccc(-c5ccccc5)cc4)cc3cc2-c2ccc3c(ccc4nc(-c5ccccc5)sc43)c21]

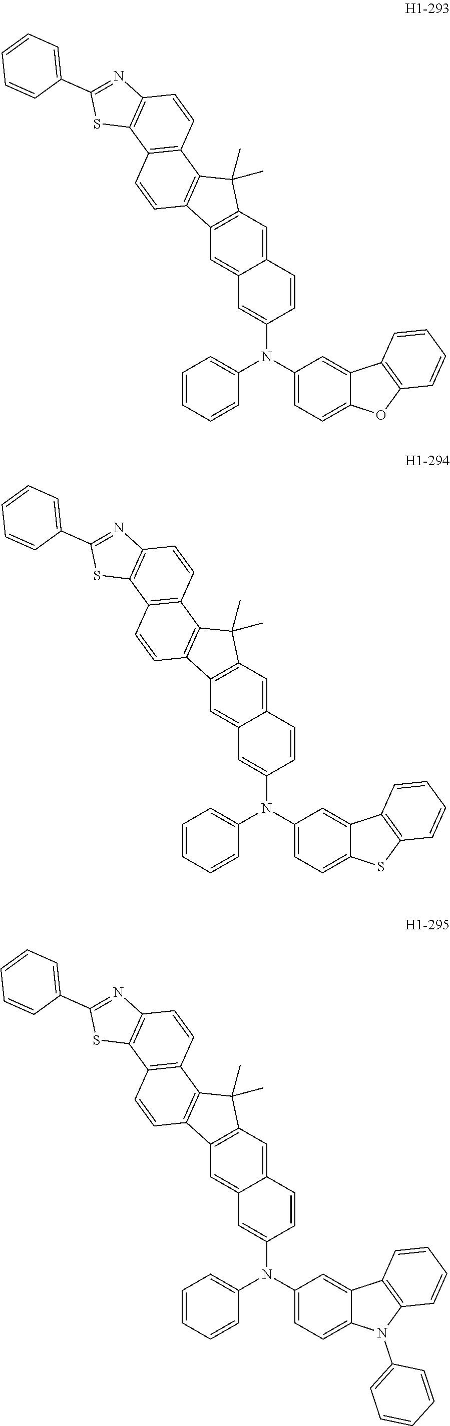 [SMILES: CC1(C)c2cc3ccc(N(c4ccccc4)c4ccc5oc6ccccc6c5c4)cc3cc2-c2ccc3c(ccc4nc(-c5ccccc5)sc43)c21 ; CC1(C)c2cc3ccc(N(c4ccccc4)c4ccc5sc6ccccc6c5c4)cc3cc2-c2ccc3c(ccc4nc(-c5ccccc5)sc43)c21 ; CC1(C)c2cc3ccc(N(c4ccccc4)c4ccc5c(c4)c4ccccc4n5-c4ccccc4)cc3cc2-c2ccc3c(ccc4nc(-c5ccccc5)sc43)c21]

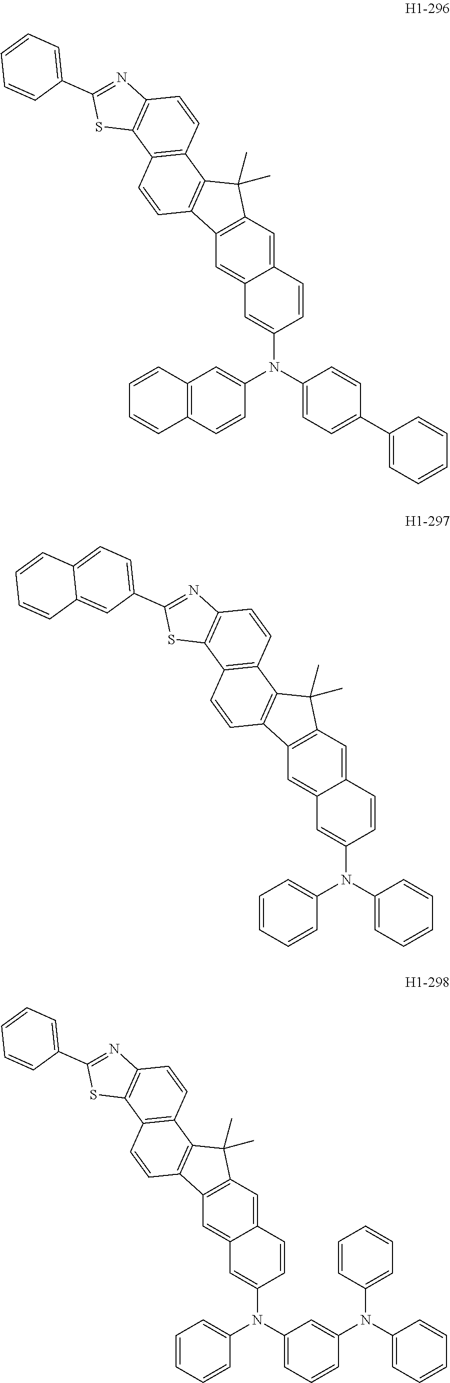 [SMILES: CC1(C)c2cc3ccc(N(c4ccc(-c5ccccc5)cc4)c4ccc5ccccc5c4)cc3cc2-c2ccc3c(ccc4nc(-c5ccccc5)sc43)c21 ; CC1(C)c2cc3ccc(N(c4ccccc4)c4ccccc4)cc3cc2-c2ccc3c(ccc4nc(-c5ccc6ccccc6c5)sc43)c21 ; CC1(C)c2cc3ccc(N(c4ccccc4)c4cccc(N(c5ccccc5)c5ccccc5)c4)cc3cc2-c2ccc3c(ccc4nc(-c5ccccc5)sc43)c21]

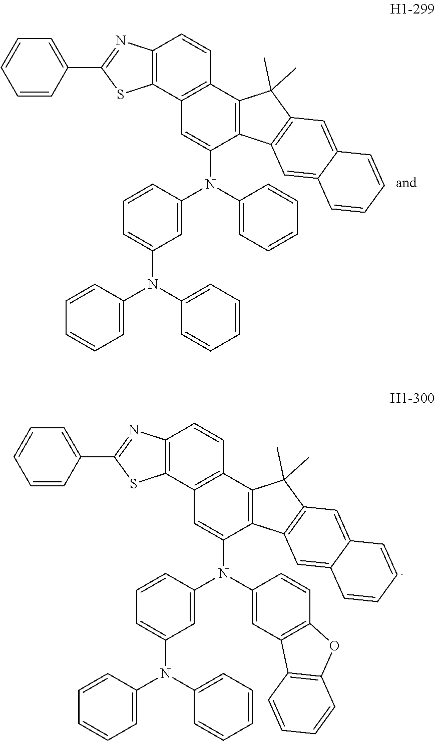 [SMILES: CC1(C)c2cc3ccccc3cc2-c2c(N(c3ccccc3)c3cccc(N(c4ccccc4)c4ccccc4)c3)cc3c(ccc4nc(-c5ccccc5)sc43)c21 ; CC1(C)c2cc3ccccc3cc2-c2c(N(c3cccc(N(c4ccccc4)c4ccccc4)c3)c3ccc4oc5ccccc5c4c3)cc3c(ccc4nc(-c5ccccc5)sc43)c21]

6. The plurality of host materials according to claim 1, wherein the compound represented by formula 2 is at least one selected from the following compounds:

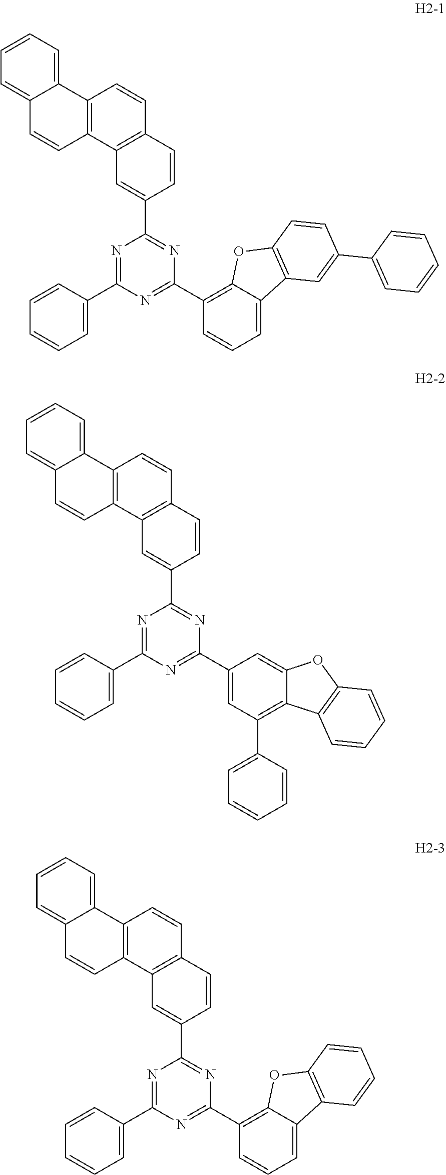 [SMILES: c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3cccc4c3oc3ccccc34)n2)cc1]

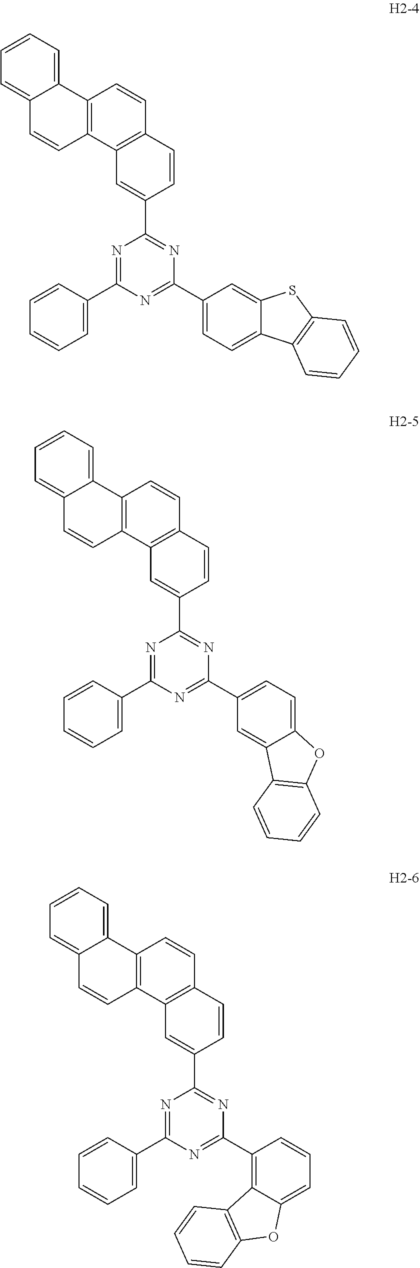 [SMILES: c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1 ; c1ccc(-c2nc(-c3ccc4ccc5c6ccccc6ccc5c4c3)nc(-c3cccc4oc5ccccc5c34)n2)cc1]

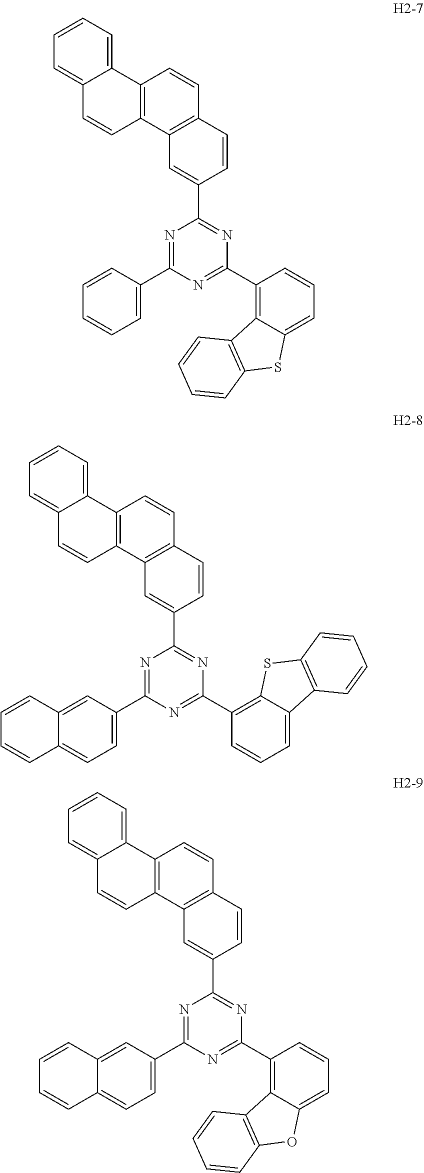

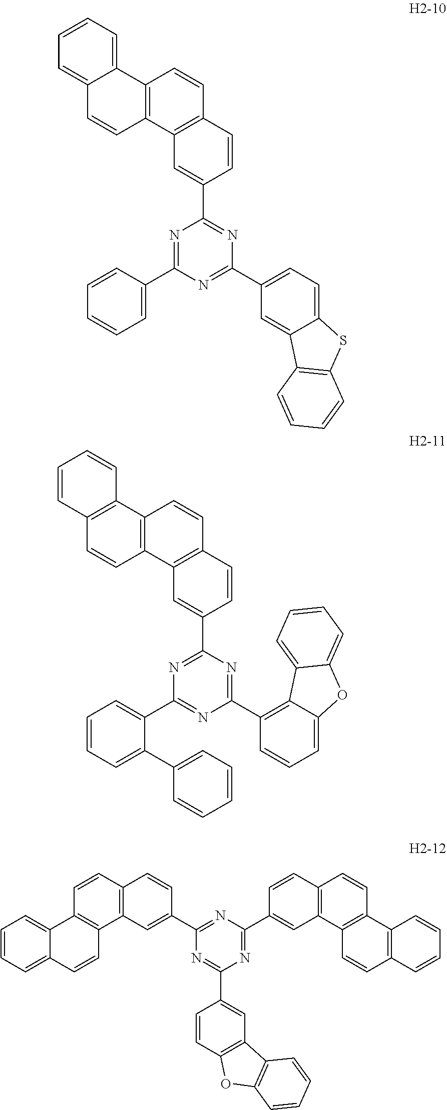 [SMILES: c1ccc(-c2nc(-c3ccc4ccc5c6ccccc6ccc5c4c3)nc(-c3ccc4sc5ccccc5c4c3)n2)cc1 ; c1ccc(-c2ccccc2-c2nc(-c3ccc4ccc5c6ccccc6ccc5c4c3)nc(-c3cccc4oc5ccccc5c34)n2)cc1 ; c1ccc2c(c1)ccc1c3cc(-c4nc(-c5ccc6ccc7c8ccccc8ccc7c6c5)nc(-c5ccc6oc7ccccc7c6c5)n4)ccc3ccc21]

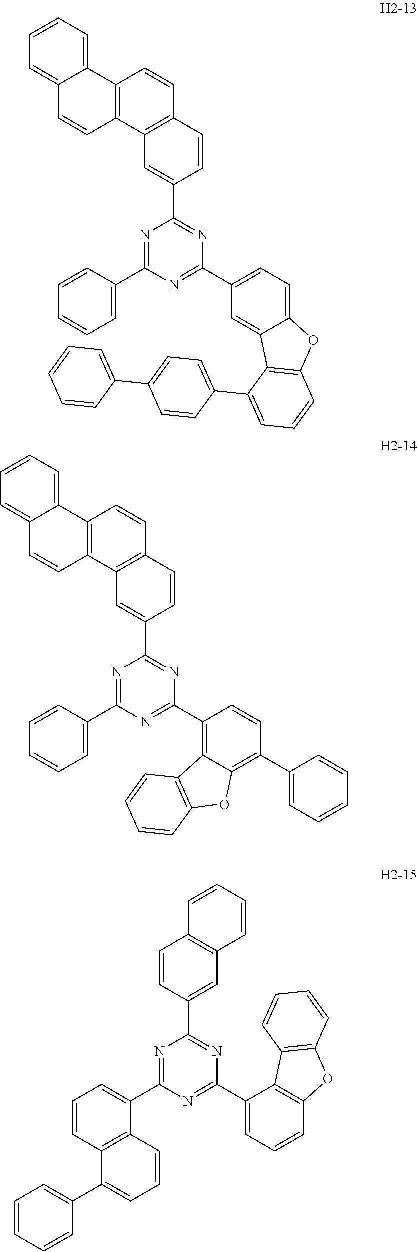 [SMILES: c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1 ; c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cccc3c(-c4nc(-c5ccc6ccccc6c5)nc(-c5cccc6oc7ccccc7c56)n4)cccc23)cc1]

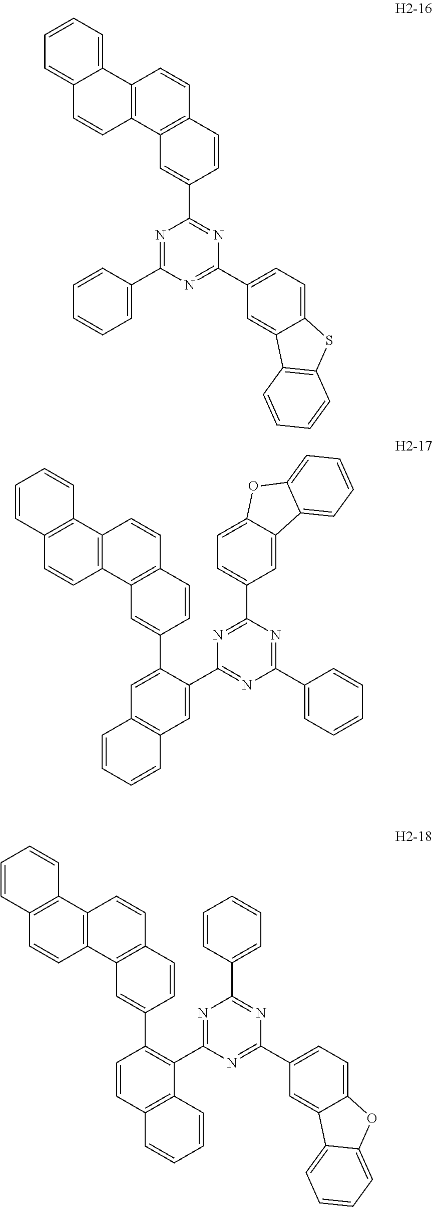

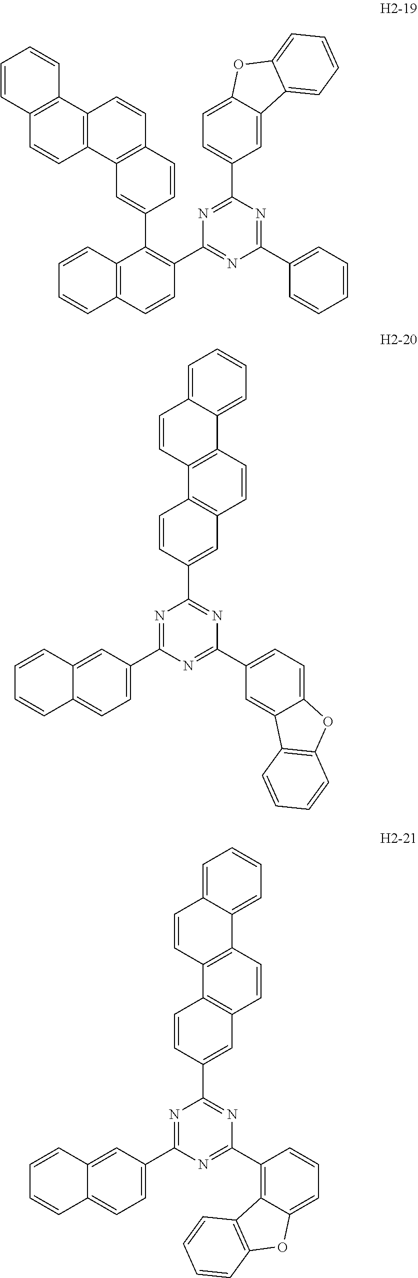 [SMILES: c1ccc(-c2nc(-c3ccc4oc5ccccc5c4c3)nc(-c3ccc4ccccc4c3-c3ccc4ccc5c6ccccc6ccc5c4c3)n2)cc1 ; c1ccc2cc(-c3nc(-c4ccc5c(ccc6c7ccccc7ccc56)c4)nc(-c4ccc5oc6ccccc6c5c4)n3)ccc2c1 ; c1ccc2cc(-c3nc(-c4ccc5c(ccc6c7ccccc7ccc56)c4)nc(-c4cccc5oc6ccccc6c45)n3)ccc2c1]

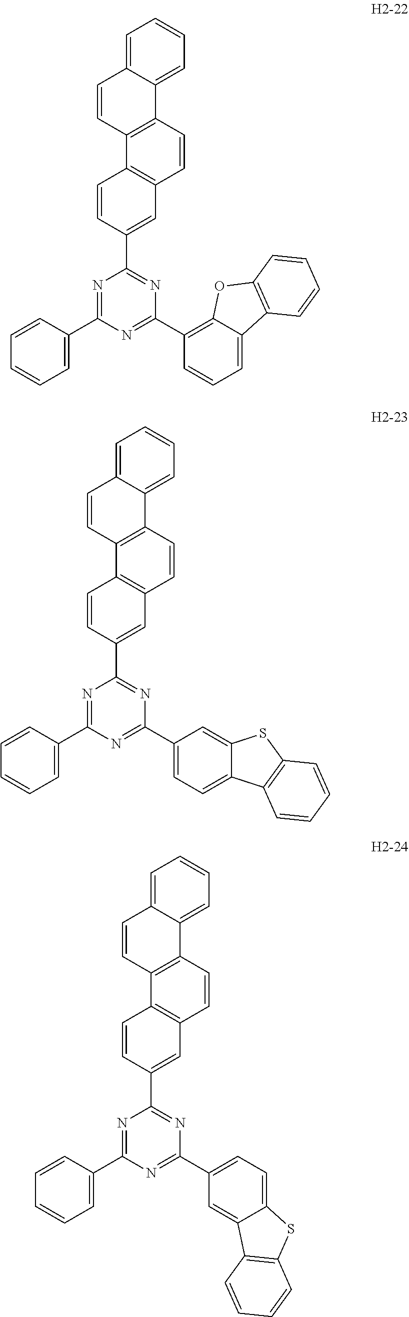 [SMILES: c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

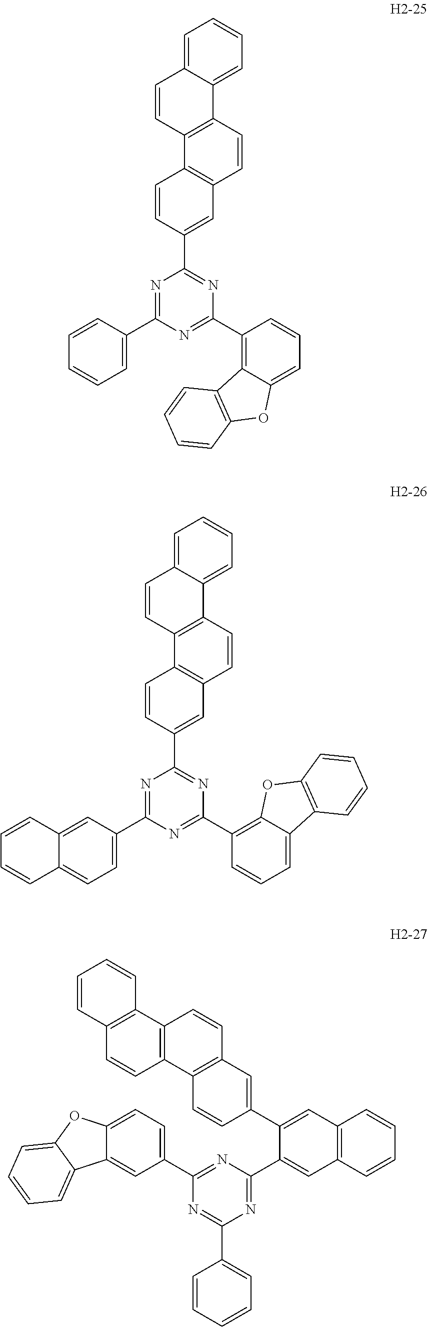 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1 ; c1ccc(-c2nc(-c3ccc4oc5ccccc5c4c3)nc(-c3cc4ccccc4cc3-c3ccc4c(ccc5c6ccccc6ccc45)c3)n2)cc1]

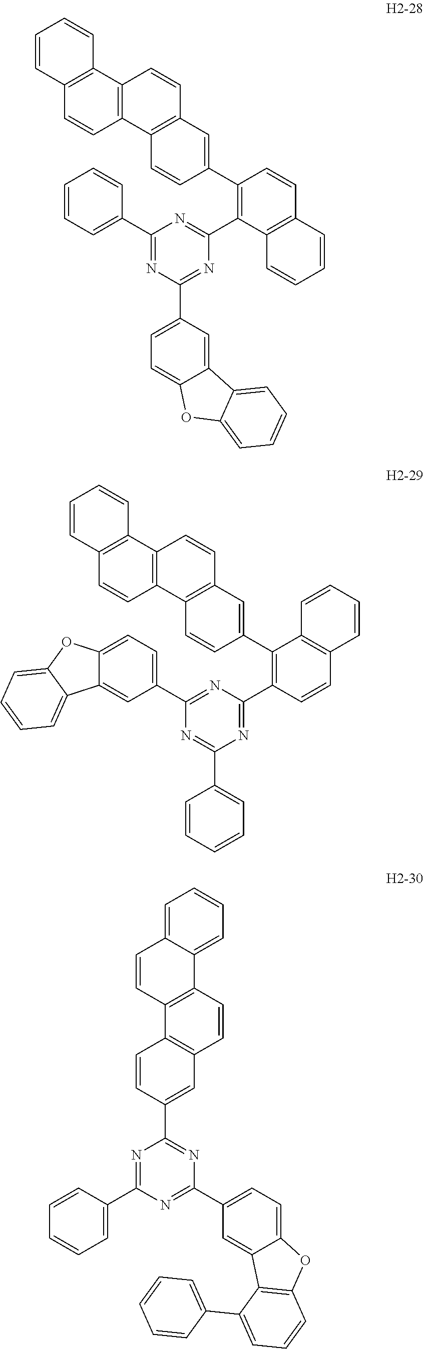

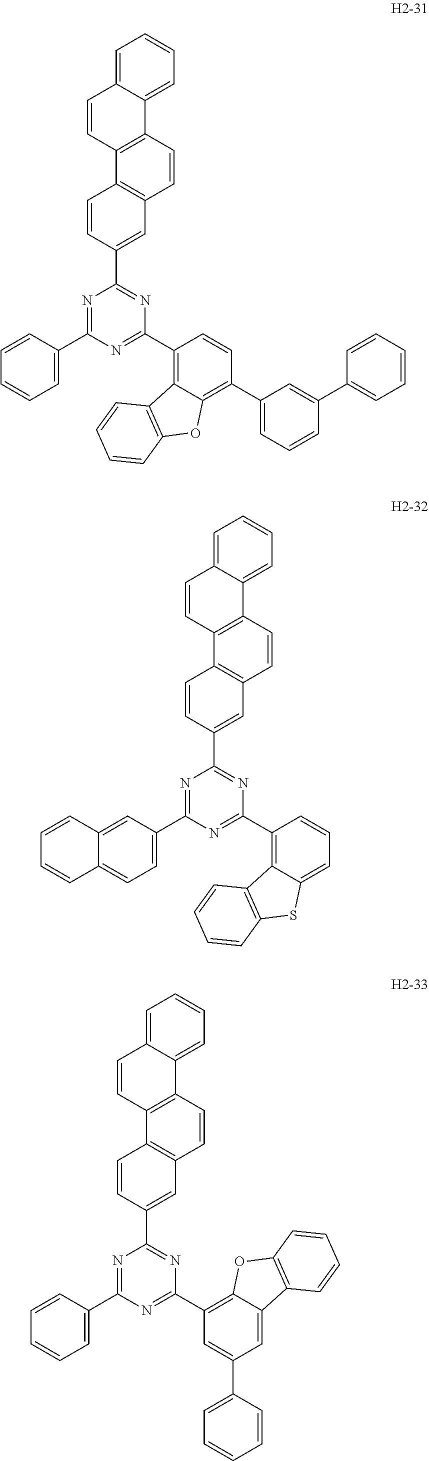 [SMILES: c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4cccc5sc6ccccc6c45)n3)ccc2c1 ; c1ccc(-c2cc(-c3nc(-c4ccccc4)nc(-c4ccc5c(ccc6c7ccccc7ccc56)c4)n3)c3oc4ccccc4c3c2)cc1]

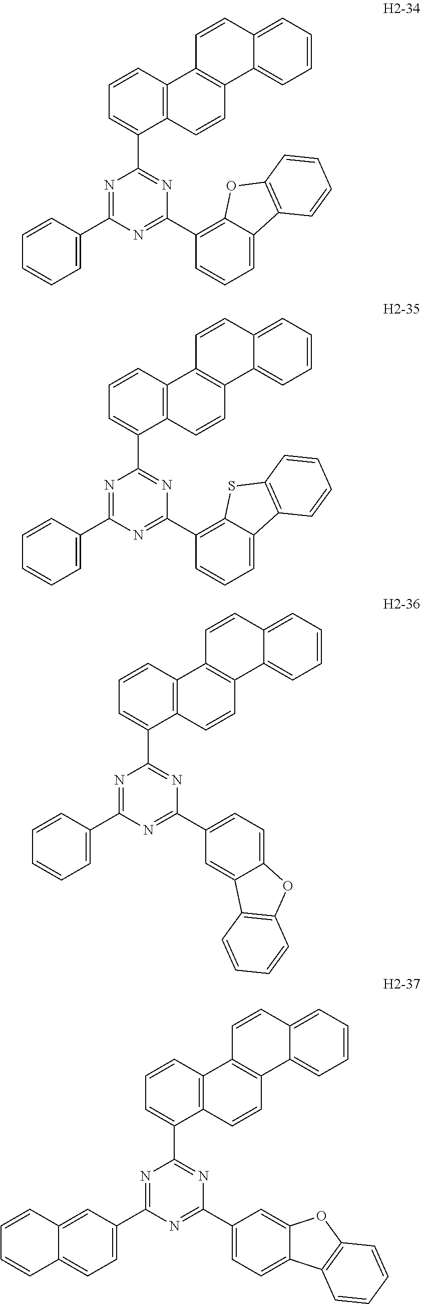

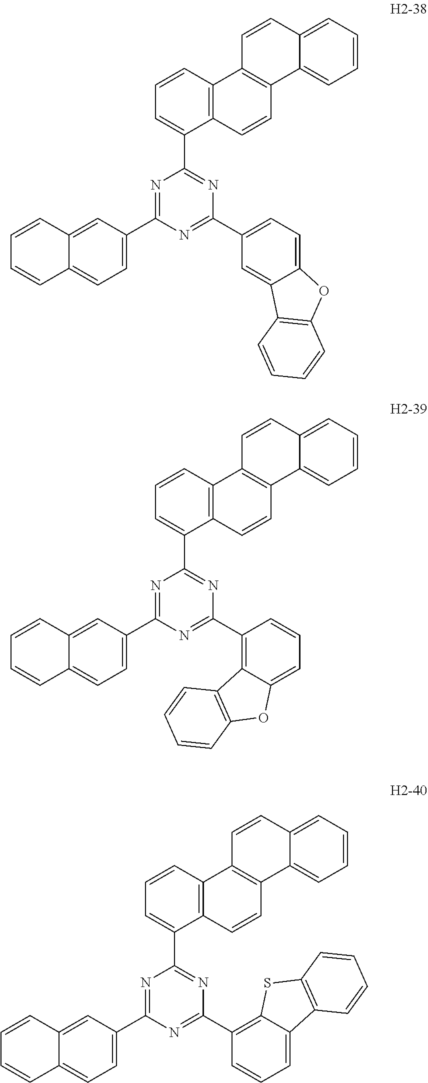

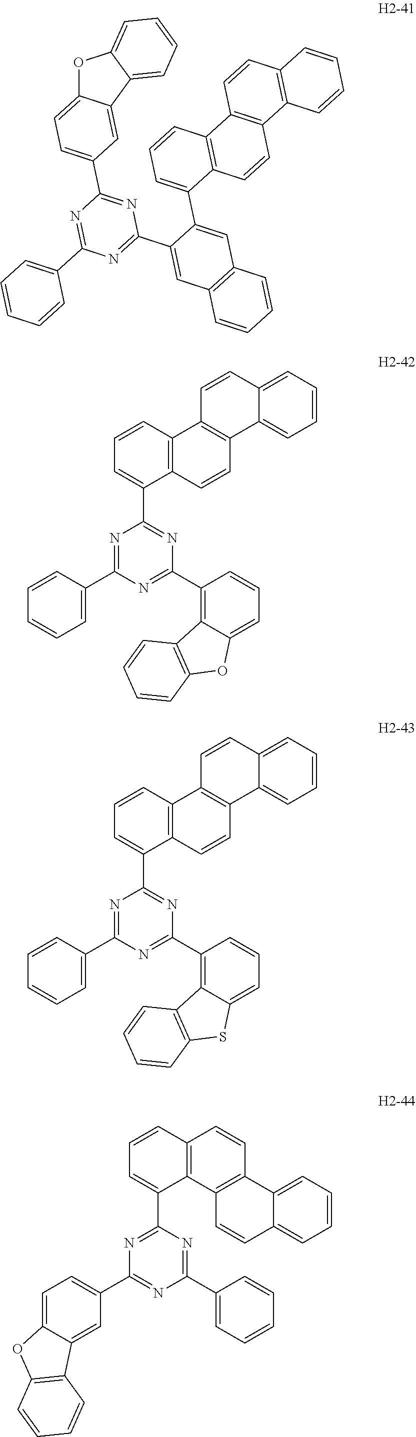

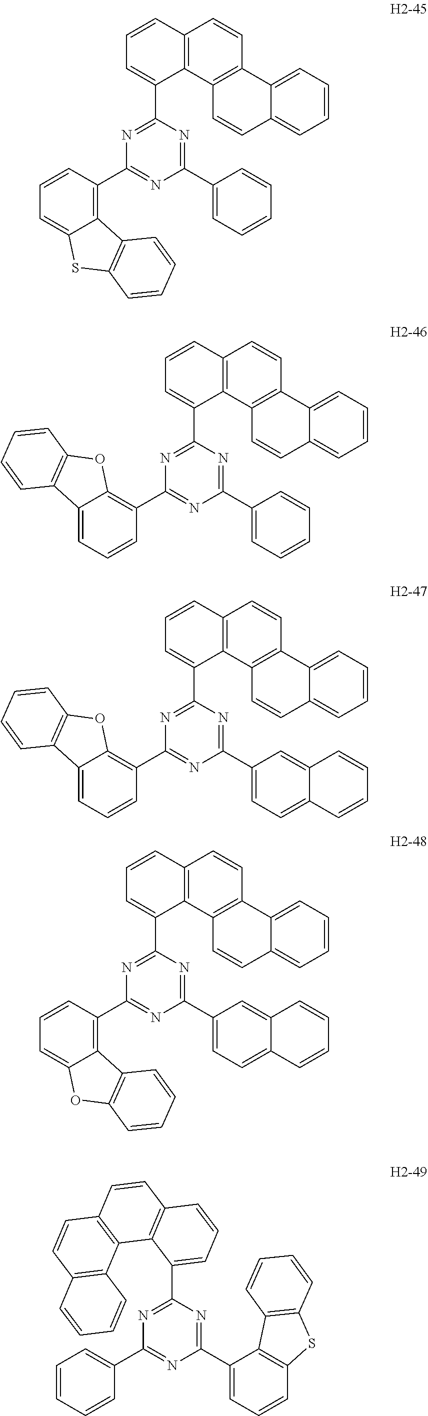 [SMILES: c1ccc(-c2nc(-c3cccc4ccc5c6ccccc6ccc5c34)nc(-c3cccc4sc5ccccc5c34)n2)cc1 ; c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1 ; c1ccc2cc(-c3nc(-c4cccc5ccc6c7ccccc7ccc6c45)nc(-c4cccc5oc6ccccc6c45)n3)ccc2c1 ; c1ccc(-c2nc(-c3cccc4sc5ccccc5c34)nc(-c3cccc4ccc5ccc6ccccc6c5c34)n2)cc1]

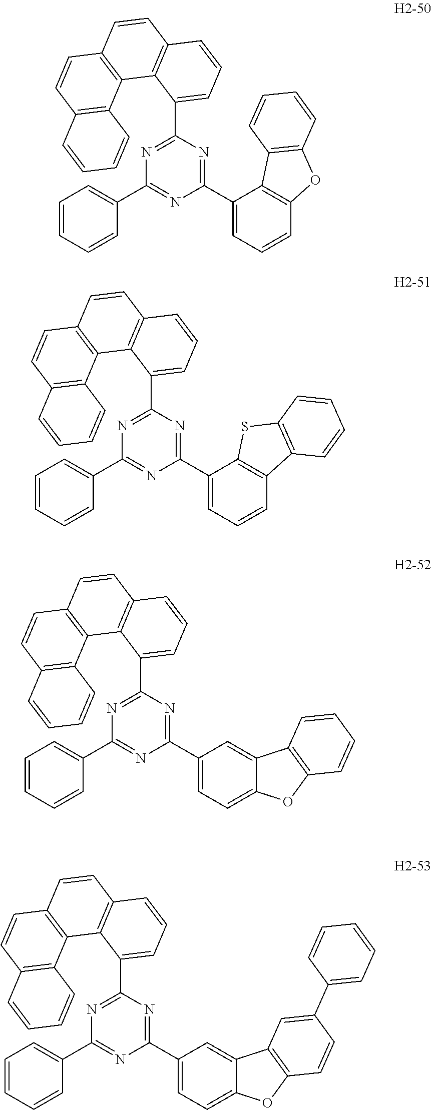

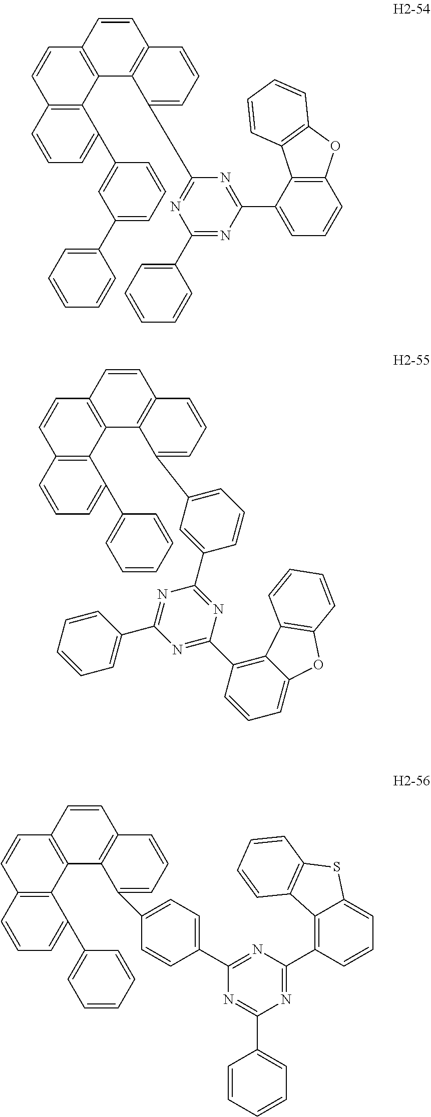 [SMILES: c1ccc(-c2cccc(-c3cccc4ccc5ccc6cccc(-c7nc(-c8ccccc8)nc(-c8cccc9oc%10ccccc%10c89)n7)c6c5c34)c2)cc1 ; c1ccc(-c2nc(-c3cccc(-c4cccc5ccc6ccc7cccc(-c8ccccc8)c7c6c45)c3)nc(-c3cccc4oc5ccccc5c34)n2)cc1 ; c1ccc(-c2nc(-c3ccc(-c4cccc5ccc6ccc7cccc(-c8ccccc8)c7c6c45)cc3)nc(-c3cccc4sc5ccccc5c34)n2)cc1]

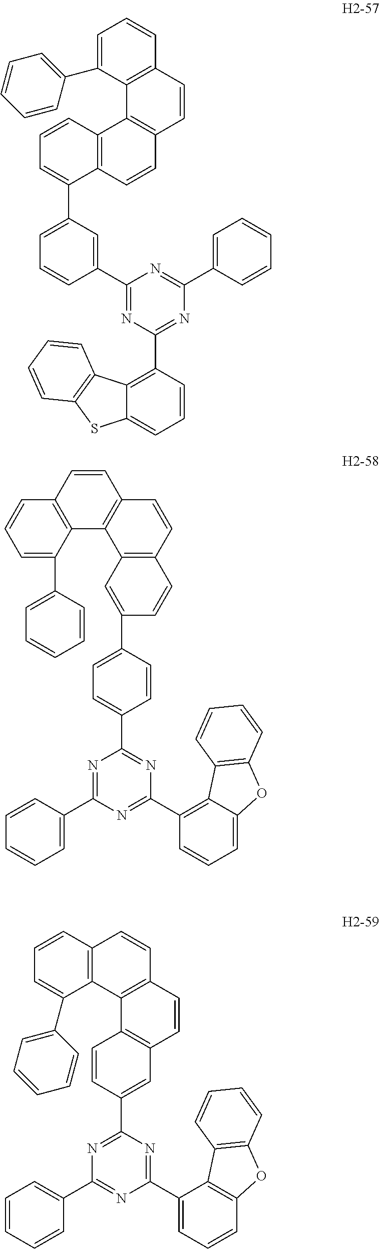

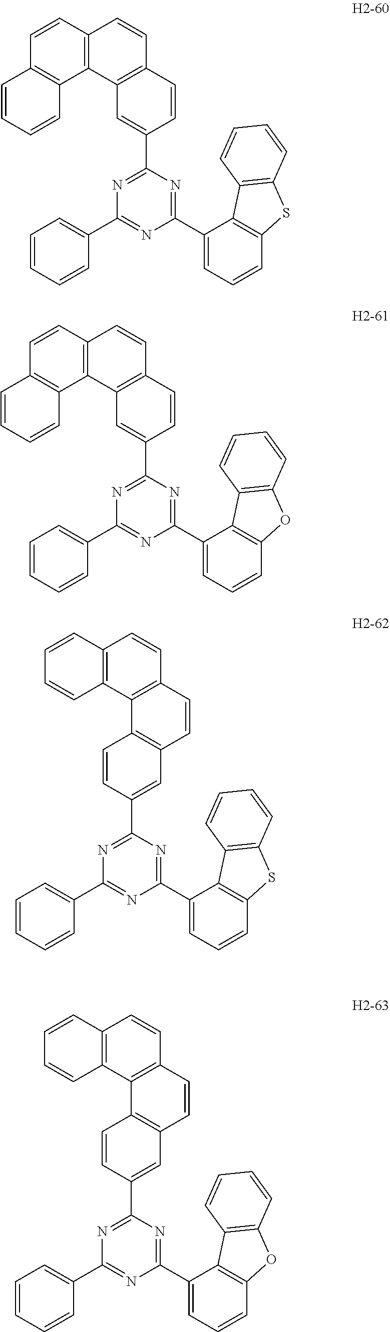

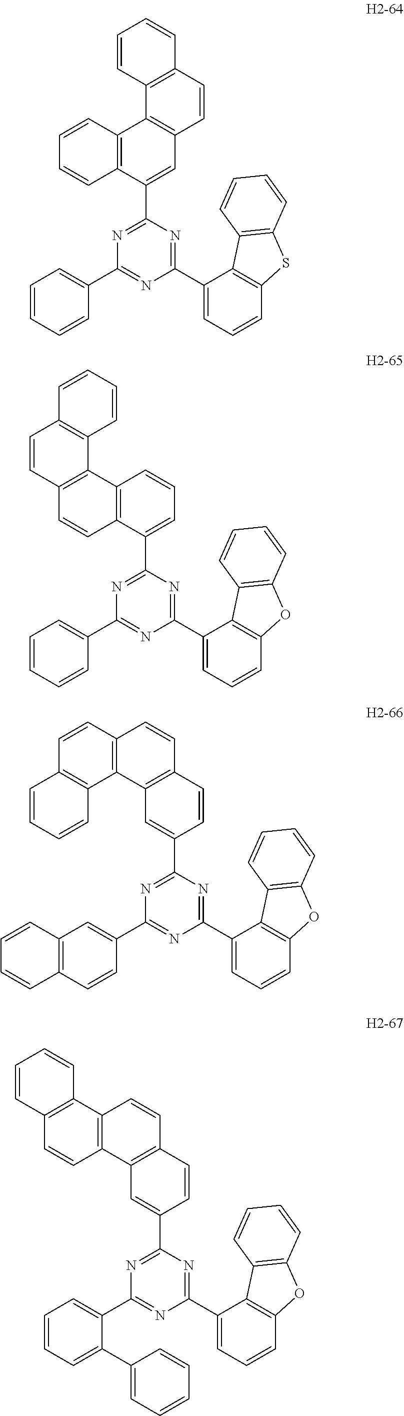

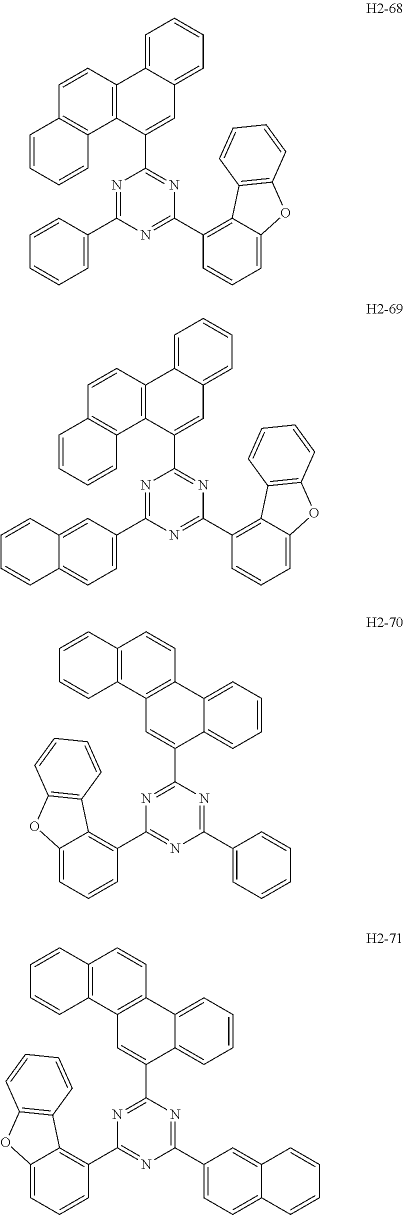

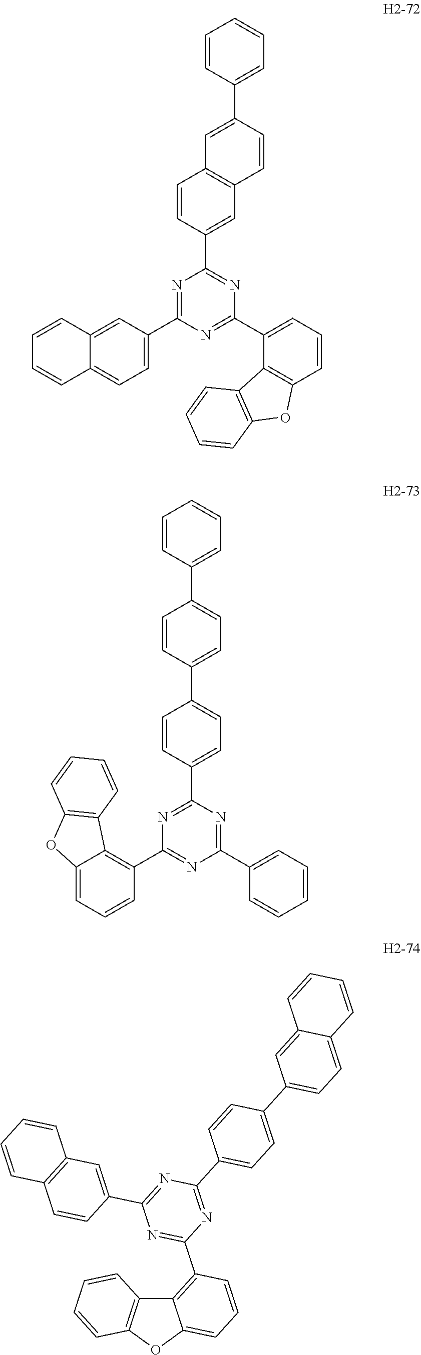 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1]

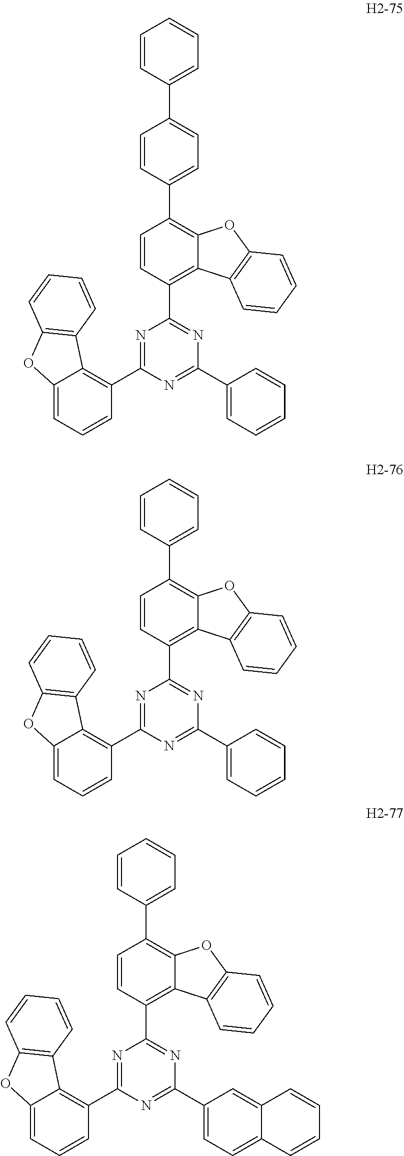 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1]

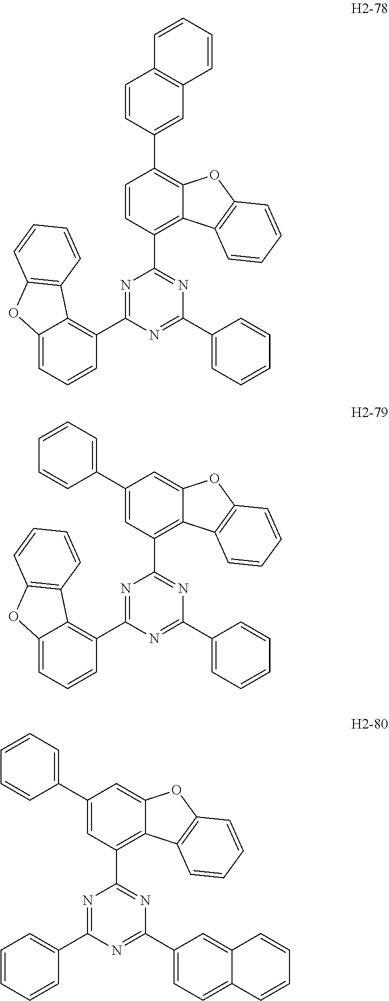

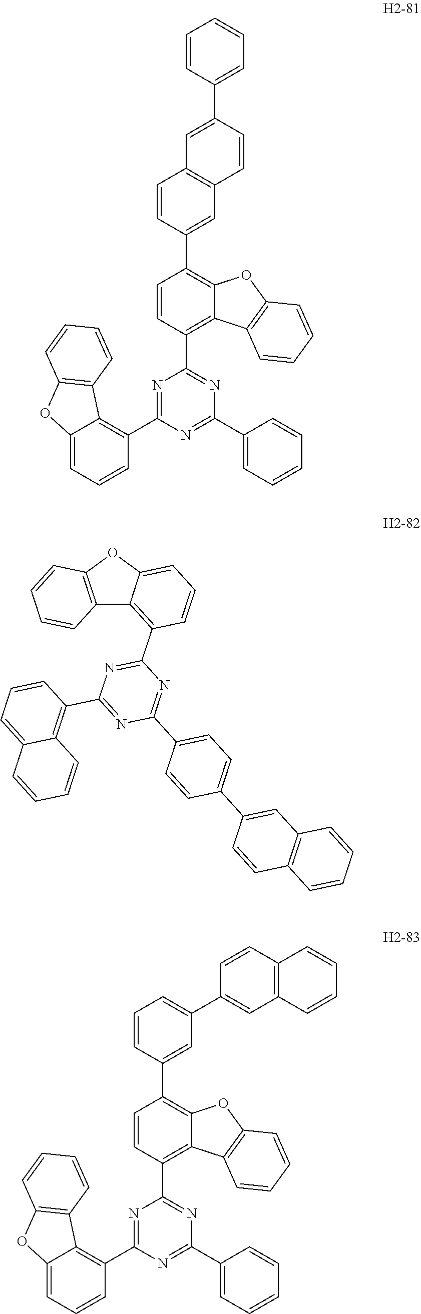 [SMILES: c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc2cc(-c3ccc(-c4nc(-c5cccc6ccccc56)nc(-c5cccc6oc7ccccc7c56)n4)cc3)ccc2c1 ; c1ccc(-c2nc(-c3cccc4oc5ccccc5c34)nc(-c3ccc(-c4cccc(-c5ccc6ccccc6c5)c4)c4oc5ccccc5c34)n2)cc1]

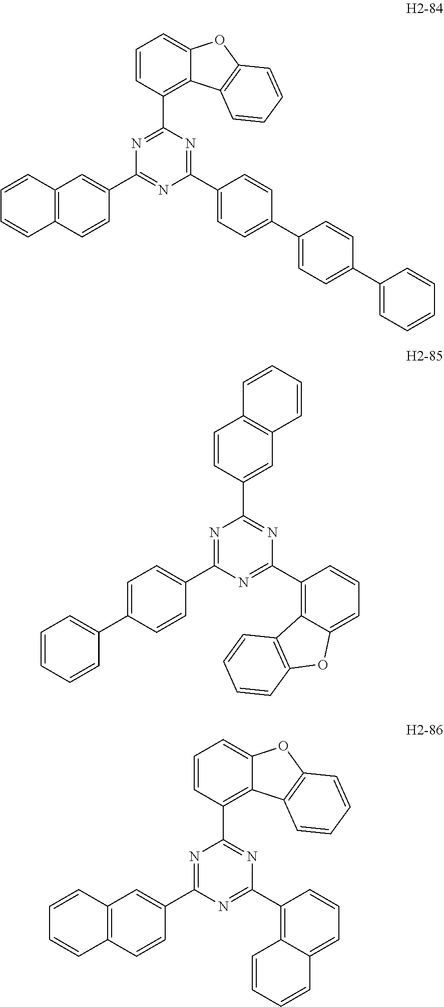 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3nc(-c4ccc5ccccc5c4)nc(-c4cccc5oc6ccccc6c45)n3)cc2)cc1 ; c1ccc2cc(-c3nc(-c4cccc5ccccc45)nc(-c4cccc5oc6ccccc6c45)n3)ccc2c1]

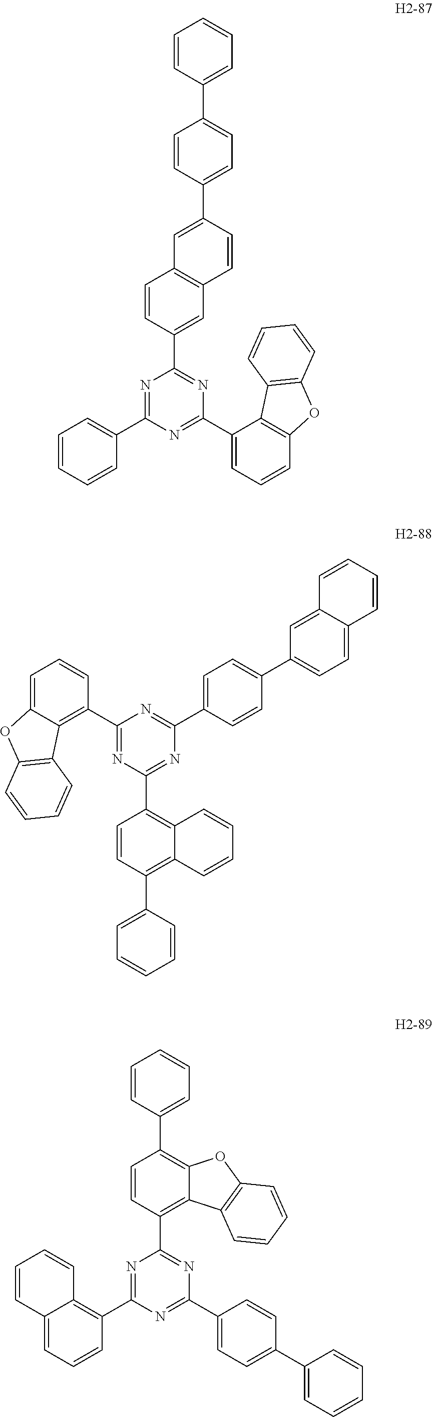 [SMILES: c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)c3ccccc23)cc1 ; c1ccc(-c2ccc(-c3nc(-c4cccc5ccccc45)nc(-c4ccc(-c5ccccc5)c5oc6ccccc6c45)n3)cc2)cc1]

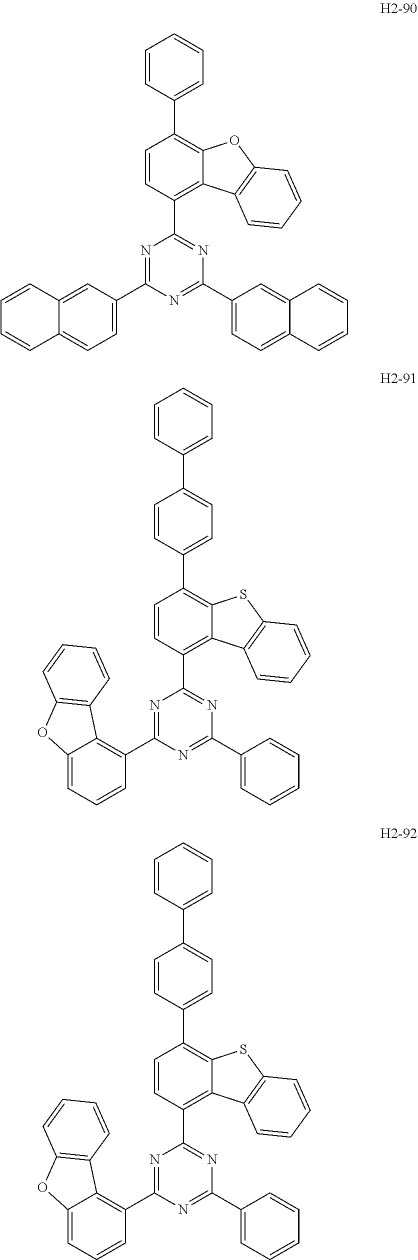 [SMILES: c1ccc(-c2ccc(-c3nc(-c4ccc5ccccc5c4)nc(-c4ccc5ccccc5c4)n3)c3c2oc2ccccc23)cc1 ; c1ccc(-c2ccc(-c3ccc(-c4nc(-c5ccccc5)nc(-c5cccc6oc7ccccc7c56)n4)c4c3sc3ccccc34)cc2)cc1 ; c1ccc(-c2ccc(-c3ccc(-c4nc(-c5ccccc5)nc(-c5cccc6oc7ccccc7c56)n4)c4c3sc3ccccc34)cc2)cc1]

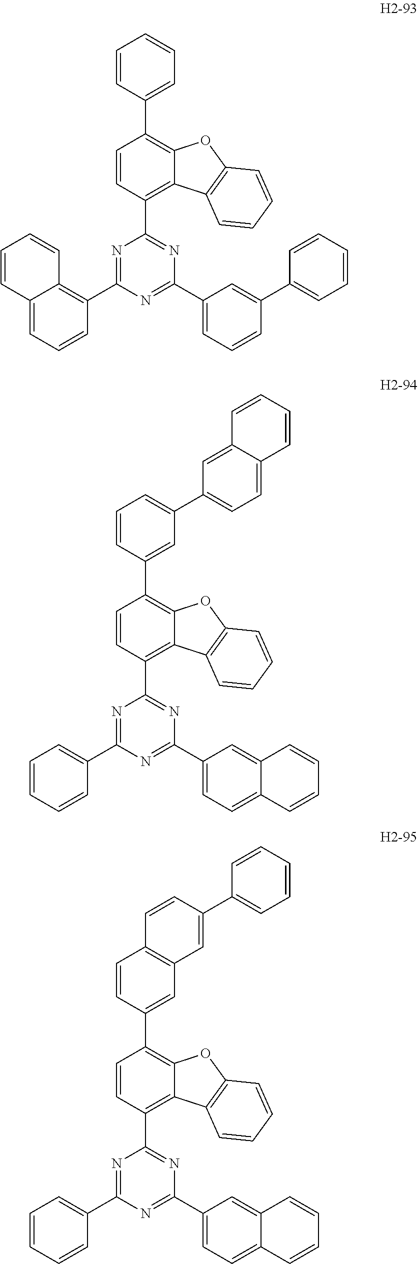

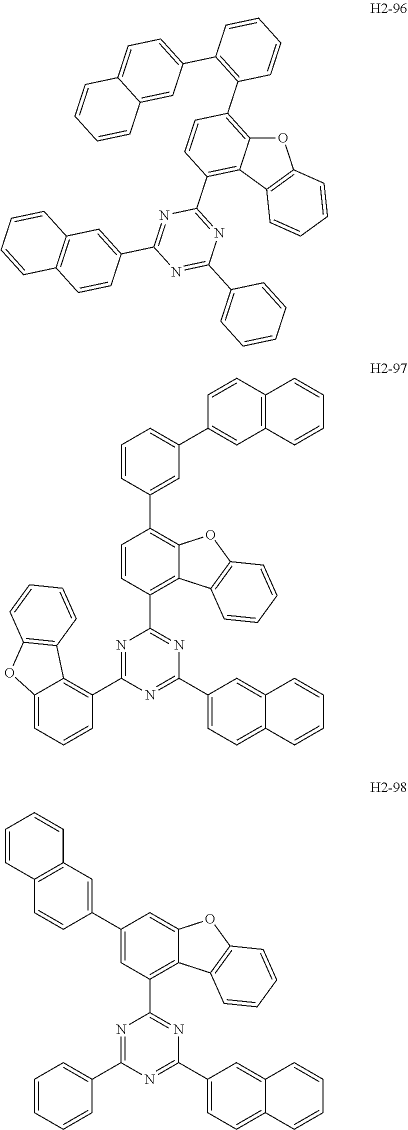

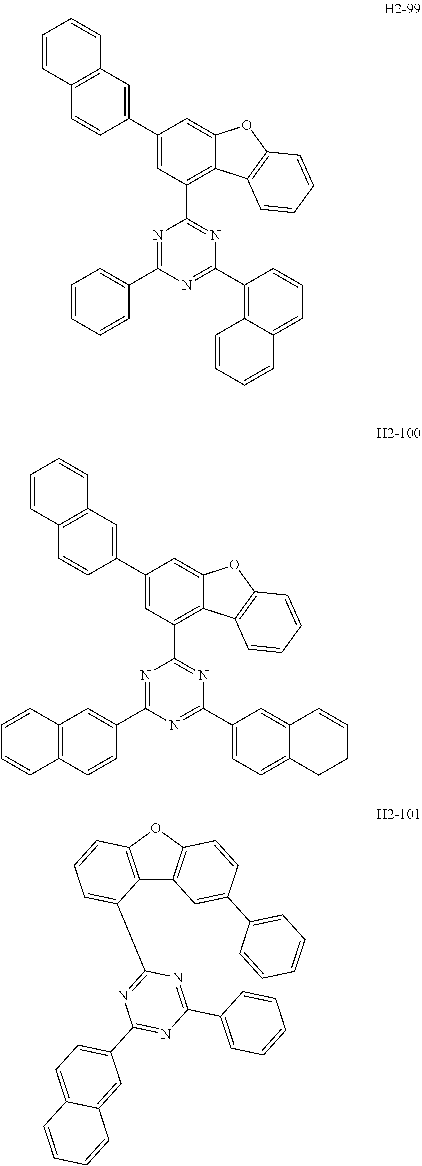 [SMILES: c1ccc(-c2nc(-c3cccc4ccccc34)nc(-c3cc(-c4ccc5ccccc5c4)cc4oc5ccccc5c34)n2)cc1 ; C1=Cc2cc(-c3nc(-c4ccc5ccccc5c4)nc(-c4cc(-c5ccc6ccccc6c5)cc5oc6ccccc6c45)n3)ccc2CC1 ; c1ccc(-c2ccc3oc4cccc(-c5nc(-c6ccccc6)nc(-c6ccc7ccccc7c6)n5)c4c3c2)cc1]

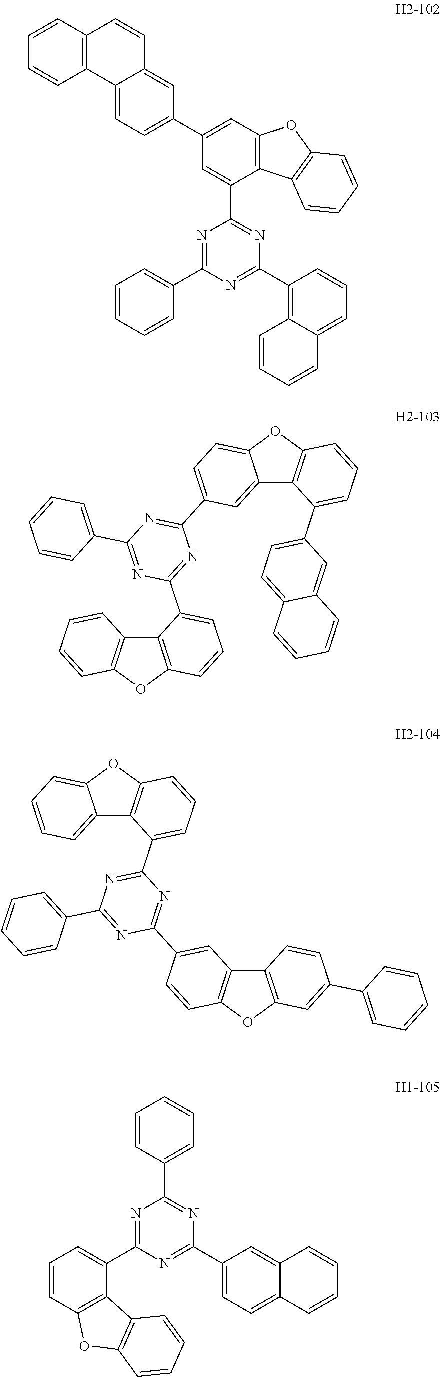 [SMILES: c1ccc(-c2nc(-c3cccc4ccccc34)nc(-c3cc(-c4ccc5c(ccc6ccccc65)c4)cc4oc5ccccc5c34)n2)cc1 ; c1ccc(-c2nc(-c3ccc4oc5cccc(-c6ccc7ccccc7c6)c5c4c3)nc(-c3cccc4oc5ccccc5c34)n2)cc1 ; c1ccc(-c2ccc3c(c2)oc2ccc(-c4nc(-c5ccccc5)nc(-c5cccc6oc7ccccc7c56)n4)cc23)cc1 ; c1ccc(-c2nc(-c3ccc4ccccc4c3)nc(-c3cccc4oc5ccccc5c34)n2)cc1]

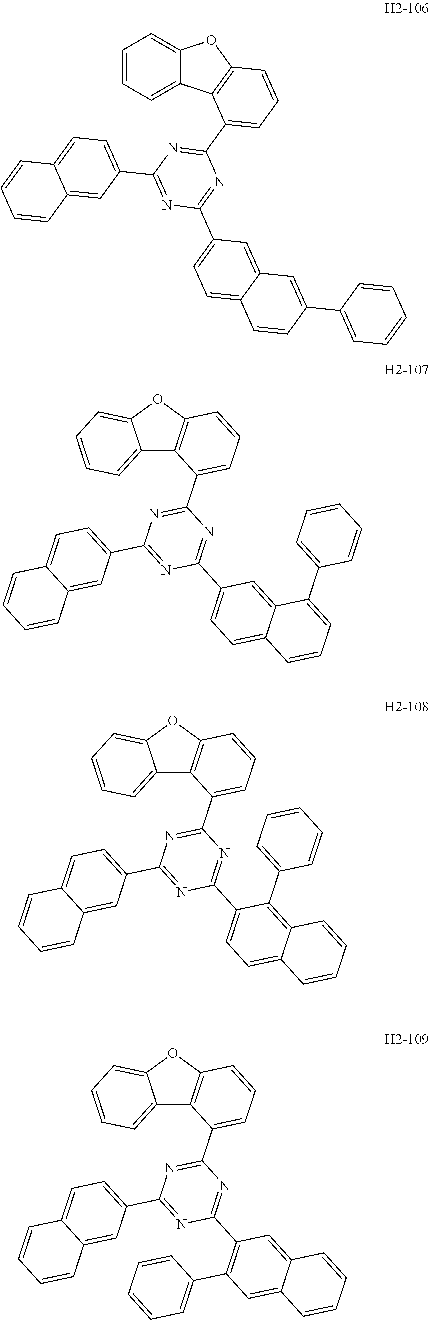 [SMILES: c1ccc(-c2ccc3ccc(-c4nc(-c5ccc6ccccc6c5)nc(-c5cccc6oc7ccccc7c56)n4)cc3c2)cc1 ; c1ccc(-c2cccc3ccc(-c4nc(-c5ccc6ccccc6c5)nc(-c5cccc6oc7ccccc7c56)n4)cc23)cc1 ; c1ccc(-c2c(-c3nc(-c4ccc5ccccc5c4)nc(-c4cccc5oc6ccccc6c45)n3)ccc3ccccc23)cc1 ; c1ccc(-c2cc3ccccc3cc2-c2nc(-c3ccc4ccccc4c3)nc(-c3cccc4oc5ccccc5c34)n2)cc1]

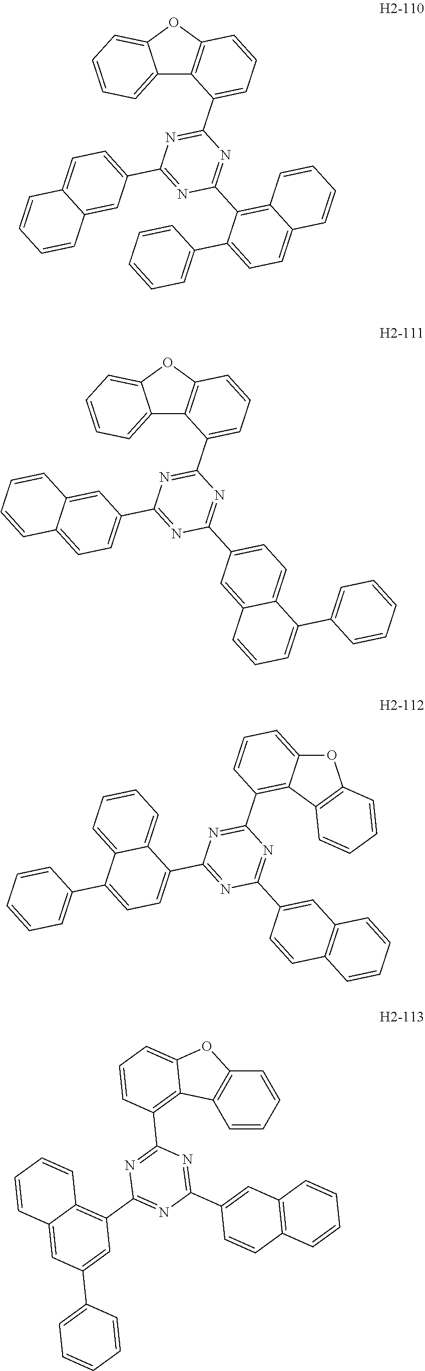 [SMILES: c1ccc(-c2ccc3ccccc3c2-c2nc(-c3ccc4ccccc4c3)nc(-c3cccc4oc5ccccc5c34)n2)cc1 ; c1ccc(-c2cccc3cc(-c4nc(-c5ccc6ccccc6c5)nc(-c5cccc6oc7ccccc7c56)n4)ccc23)cc1 ; c1ccc(-c2ccc(-c3nc(-c4ccc5ccccc5c4)nc(-c4cccc5oc6ccccc6c45)n3)c3ccccc23)cc1 ; c1ccc(-c2cc(-c3nc(-c4ccc5ccccc5c4)nc(-c4cccc5oc6ccccc6c45)n3)c3ccccc3c2)cc1]

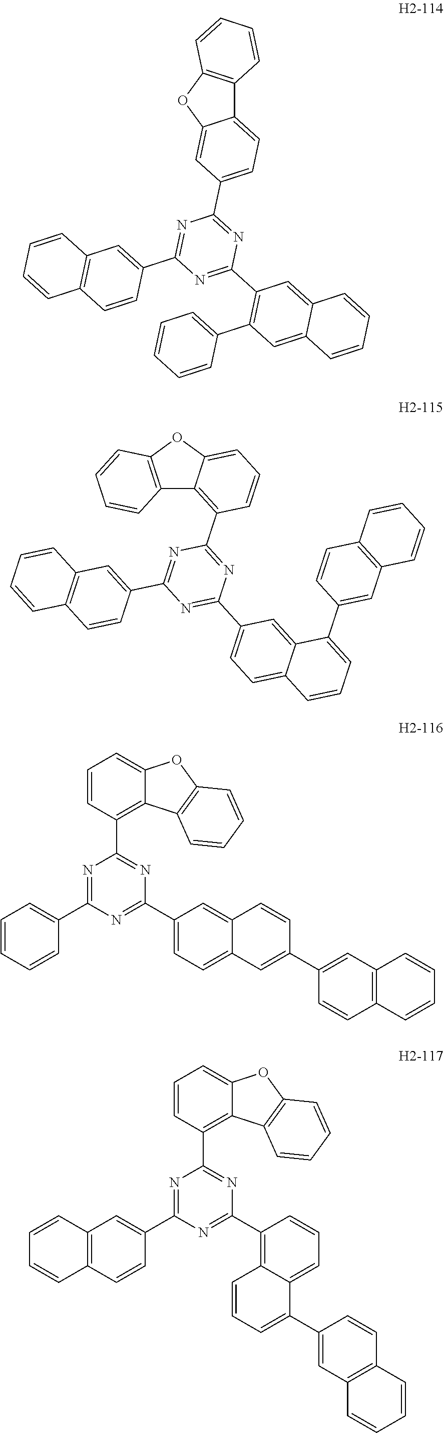 [SMILES: c1ccc(-c2cc3ccccc3cc2-c2nc(-c3ccc4ccccc4c3)nc(-c3ccc4c(c3)oc3ccccc34)n2)cc1 ; c1ccc2cc(-c3nc(-c4ccc5cccc(-c6ccc7ccccc7c6)c5c4)nc(-c4cccc5oc6ccccc6c45)n3)ccc2c1 ; c1ccc(-c2nc(-c3ccc4cc(-c5ccc6ccccc6c5)ccc4c3)nc(-c3cccc4oc5ccccc5c34)n2)cc1 ; c1ccc2cc(-c3nc(-c4cccc5c(-c6ccc7ccccc7c6)cccc45)nc(-c4cccc5oc6ccccc6c45)n3)ccc2c1]

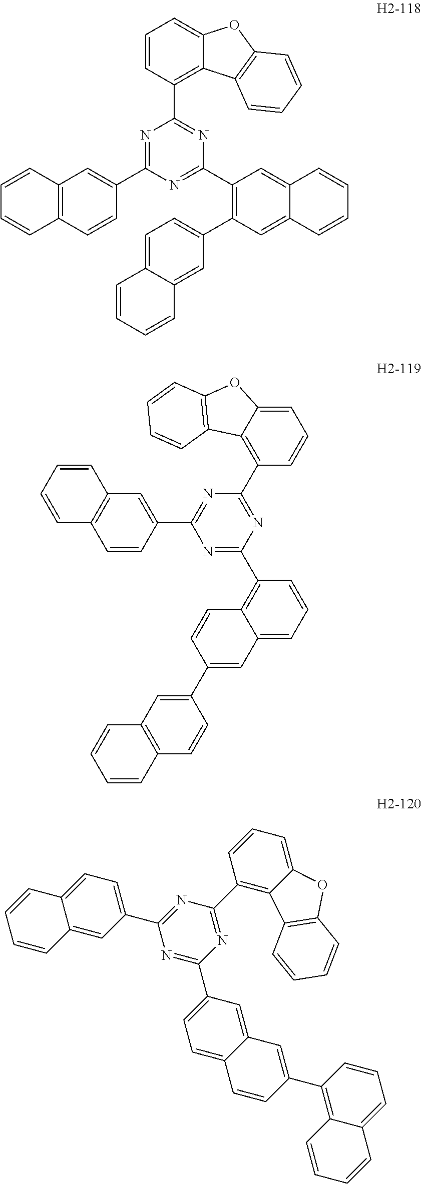 [SMILES: c1ccc2cc(-c3nc(-c4cc5ccccc5cc4-c4ccc5ccccc5c4)nc(-c4cccc5oc6ccccc6c45)n3)ccc2c1 ; c1ccc2cc(-c3ccc4c(-c5nc(-c6ccc7ccccc7c6)nc(-c6cccc7oc8ccccc8c67)n5)cccc4c3)ccc2c1 ; c1ccc2cc(-c3nc(-c4ccc5ccc(-c6cccc7ccccc67)cc5c4)nc(-c4cccc5oc6ccccc6c45)n3)ccc2c1]

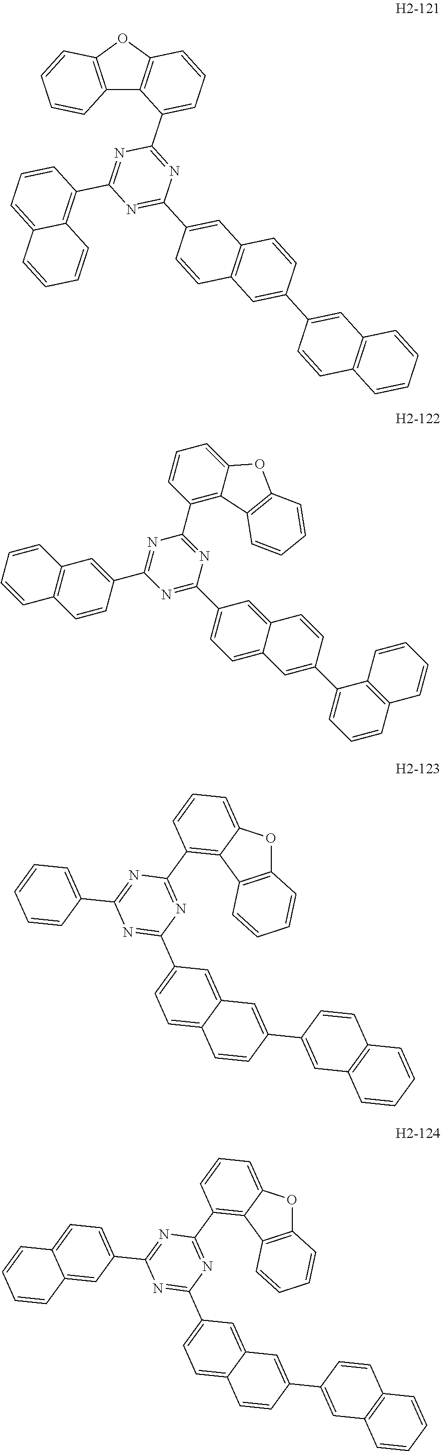 [SMILES: c1ccc2cc(-c3ccc4cc(-c5nc(-c6cccc7ccccc67)nc(-c6cccc7oc8ccccc8c67)n5)ccc4c3)ccc2c1 ; c1ccc2cc(-c3nc(-c4ccc5cc(-c6cccc7ccccc67)ccc5c4)nc(-c4cccc5oc6ccccc6c45)n3)ccc2c1 ; c1ccc(-c2nc(-c3ccc4ccc(-c5ccc6ccccc6c5)cc4c3)nc(-c3cccc4oc5ccccc5c34)n2)cc1 ; c1ccc2cc(-c3ccc4ccc(-c5nc(-c6ccc7ccccc7c6)nc(-c6cccc7oc8ccccc8c67)n5)cc4c3)ccc2c1]

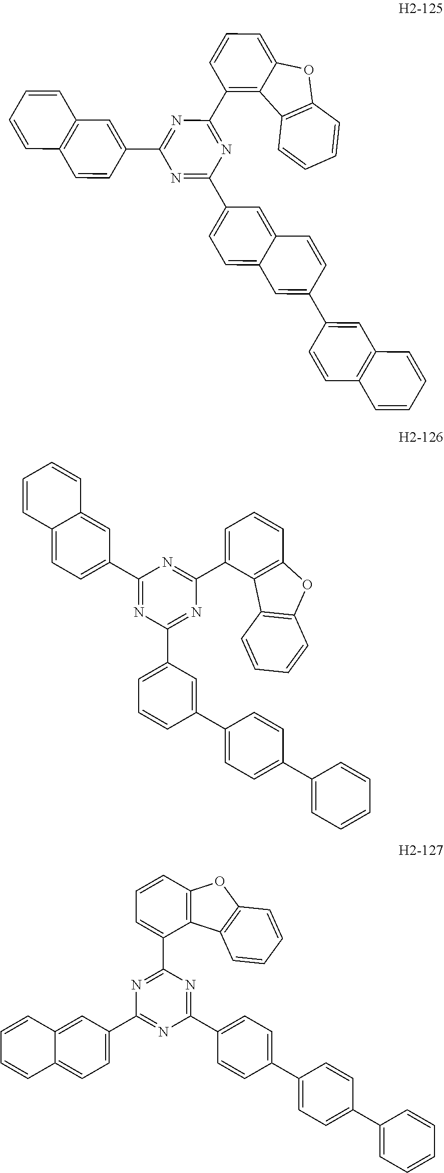 [SMILES: c1ccc2cc(-c3ccc4cc(-c5nc(-c6ccc7ccccc7c6)nc(-c6cccc7oc8ccccc8c67)n5)ccc4c3)ccc2c1 ; c1ccc(-c2ccc(-c3cccc(-c4nc(-c5ccc6ccccc6c5)nc(-c5cccc6oc7ccccc7c56)n4)c3)cc2)cc1 ; c1ccc(-c2ccc(-c3ccc(-c4nc(-c5ccc6ccccc6c5)nc(-c5cccc6oc7ccccc7c56)n4)cc3)cc2)cc1]

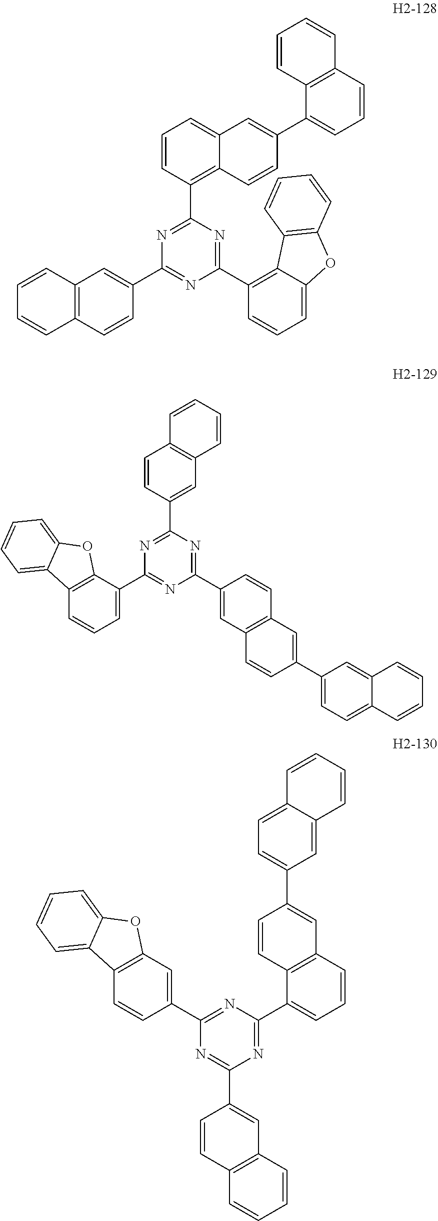

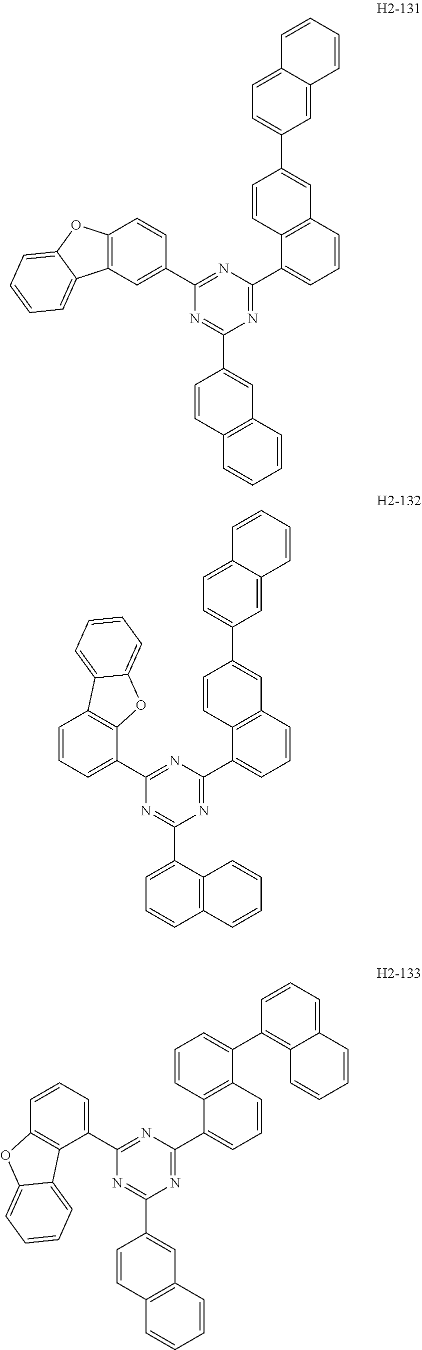

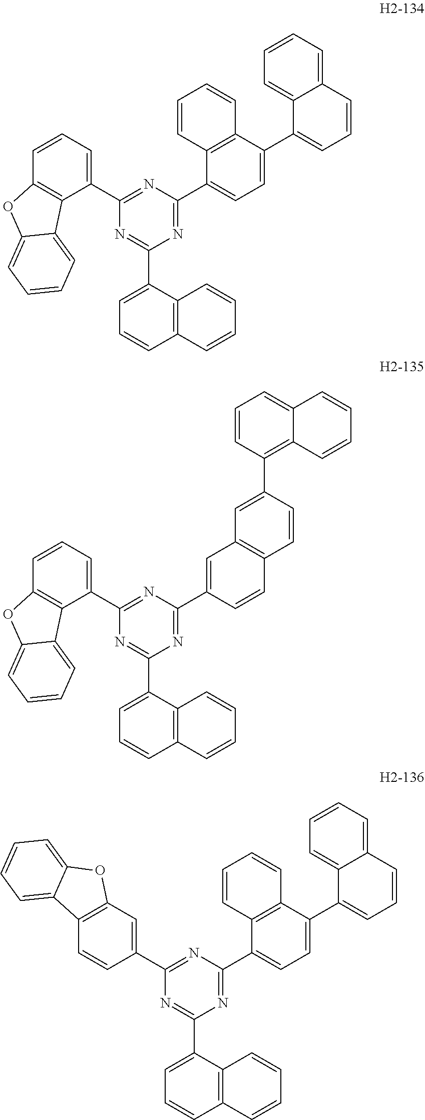 [SMILES: c1ccc2c(-c3nc(-c4ccc(-c5cccc6ccccc56)c5ccccc45)nc(-c4cccc5oc6ccccc6c45)n3)cccc2c1 ; c1ccc2c(-c3ccc4ccc(-c5nc(-c6cccc7ccccc67)nc(-c6cccc7oc8ccccc8c67)n5)cc4c3)cccc2c1 ; c1ccc2c(-c3nc(-c4ccc5c(c4)oc4ccccc45)nc(-c4ccc(-c5cccc6ccccc56)c5ccccc45)n3)cccc2c1]

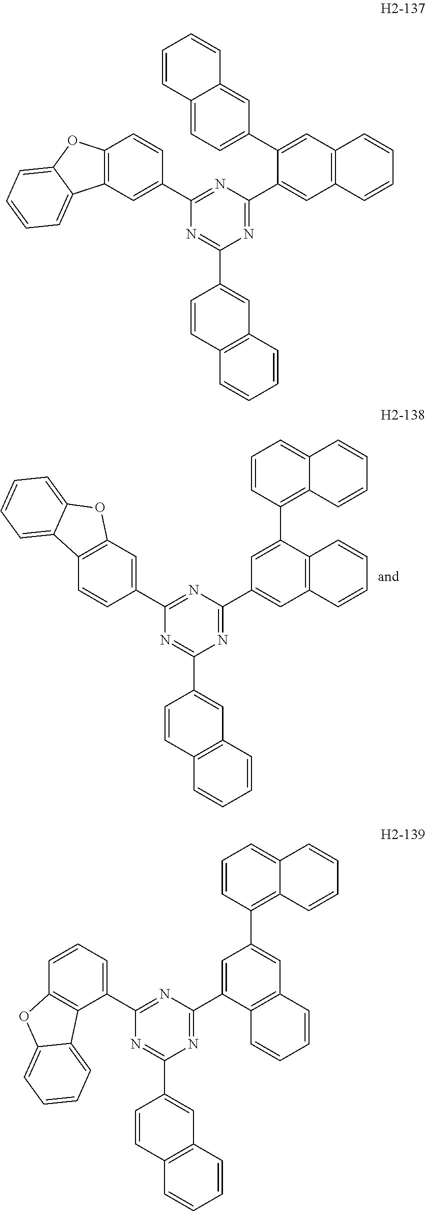 [SMILES: c1ccc2cc(-c3nc(-c4ccc5oc6ccccc6c5c4)nc(-c4cc5ccccc5cc4-c4ccc5ccccc5c4)n3)ccc2c1 ; c1ccc2cc(-c3nc(-c4cc(-c5cccc6ccccc56)c5ccccc5c4)nc(-c4ccc5c(c4)oc4ccccc45)n3)ccc2c1 ; c1ccc2cc(-c3nc(-c4cc(-c5cccc6ccccc56)cc5ccccc45)nc(-c4cccc5oc6ccccc6c45)n3)ccc2c1]

7. An organic electroluminescent device comprising an anode; a cathode; and at least one light-emitting layer between the anode and the cathode, wherein the at least one light-emitting layer comprises the plurality of host materials according to claim 1.

8. An organic electroluminescent compound represented by the following formula 1′: 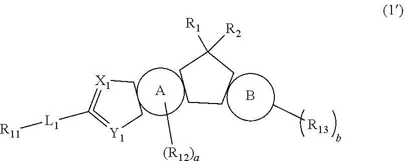
in formula 1′,
R₁ and R₂ each independently represent a substituted or unsubstituted (C1-C30)alkyl, a substituted or unsubstituted (C6-C30)aryl, or a substituted or unsubstituted (3- to 30-membered)heteroaryl; or may be linked to each other to form a ring(s);
X₁ and Y₁ each independently represent —N═, —NR₁₄, —O— or —S—, with a proviso that any one of X₁ and Y₁ represents —N═, and the other of X₁ and Y₁ represents —NR₁₄—, —O— or —S—;
ring A and ring B each independently represent a substituted or unsubstituted (C6-C30)arene, or a substituted or unsubstituted (3- to 30-membered)heteroarene;
R₁₁ and R₁₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or the formula 1′-1 or 1′-2, with a proviso that at least one of R₁₁ to R₁₄ is represented by the following formula 1′-1 or 1′-2, where if R₁₁ is represented by the following formula 1′-1 or 1′-2, R₁₁ is represented by the formula 1′-2; 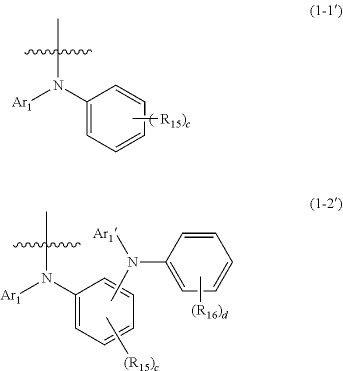
R₁₅ and R₁₆ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₂-N(Ar₂)(Ar₃);
L₁ and L₂ each independently represent a single bond, a substituted or unsubstituted (C6-C30)arylene, or a substituted or unsubstituted (3- to 30-membered)heteroarylene;
Ar₁ to Ar₃ and Ar₁′ each independently represent hydrogen, deuterium, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; and
a represents an integer of 1 to 6, b represents an integer of 1 to 8, c′ represents an integer of 1 to 4, c and d represent an integer of 1 to 5, where if a to d and c′ are an integer of 2 or more, each of R₁₂, each of R₁₃, each of R₁₅, and each of R₁₆ may be the same as, or different from, each other.

9. The organic electroluminescent compound according to claim 8, wherein formula 1′ is represented by one of the following formulas 3′ and 4′: 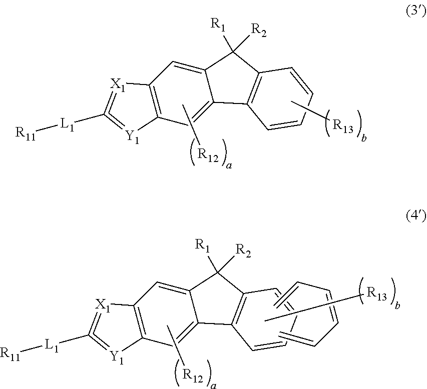
in the formulas 3′ and 4′,
R₁, R₂, R₁₁ to R₁₃, X₁, Y₁, and L₁ are as defined in claim 8, a represents an integer of 1 or 2, and b represents an integer of 1 to 6.

10. The organic electroluminescent compound according to claim 8, wherein the organic electroluminescent compound represented by formula 1′ is selected from the following compounds:

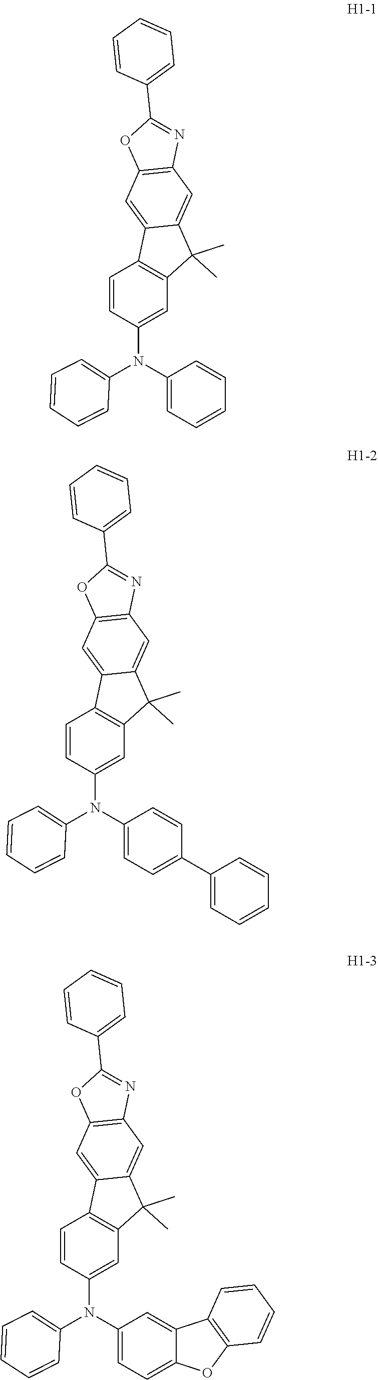

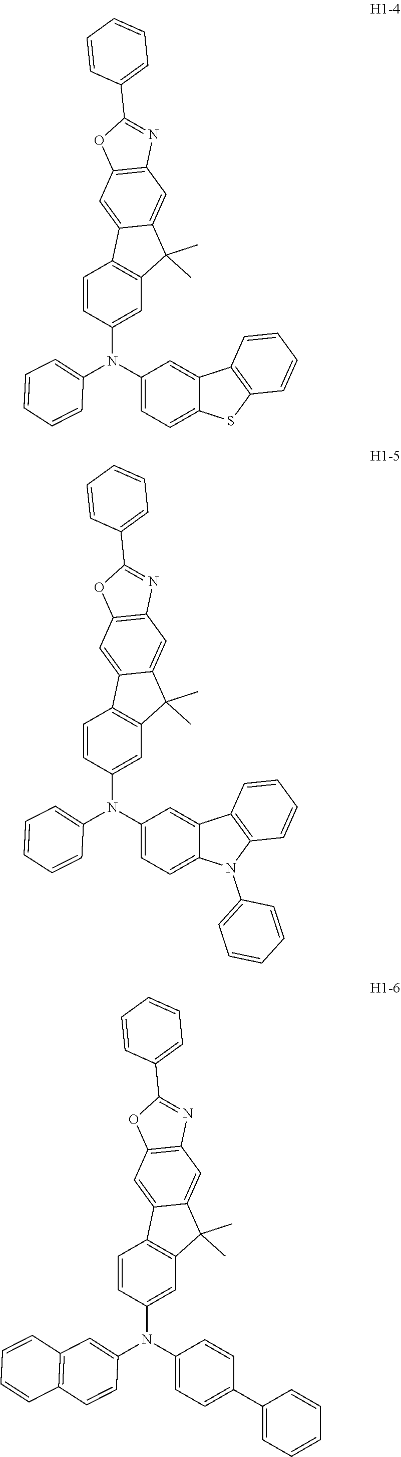 [SMILES: CC1(C)c2cc(N(c3ccccc3)c3ccc4sc5ccccc5c4c3)ccc2-c2cc3oc(-c4ccccc4)nc3cc21 ; CC1(C)c2cc(N(c3ccccc3)c3ccc4c(c3)c3ccccc3n4-c3ccccc3)ccc2-c2cc3oc(-c4ccccc4)nc3cc21 ; CC1(C)c2cc(N(c3ccc(-c4ccccc4)cc3)c3ccc4ccccc4c3)ccc2-c2cc3oc(-c4ccccc4)nc3cc21]

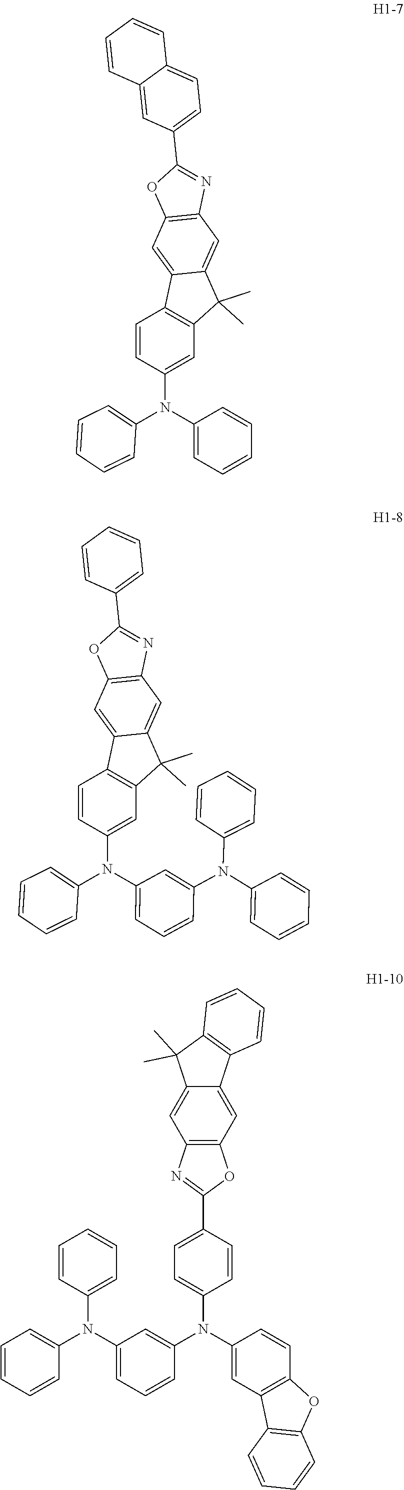 [SMILES: CC1(C)c2cc(N(c3ccccc3)c3ccccc3)ccc2-c2cc3oc(-c4ccc5ccccc5c4)nc3cc21 ; CC1(C)c2cc(N(c3ccccc3)c3cccc(N(c4ccccc4)c4ccccc4)c3)ccc2-c2cc3oc(-c4ccccc4)nc3cc21 ; CC1(C)c2ccccc2-c2cc3oc(-c4ccc(N(c5cccc(N(c6ccccc6)c6ccccc6)c5)c5ccc6oc7ccccc7c6c5)cc4)nc3cc21]

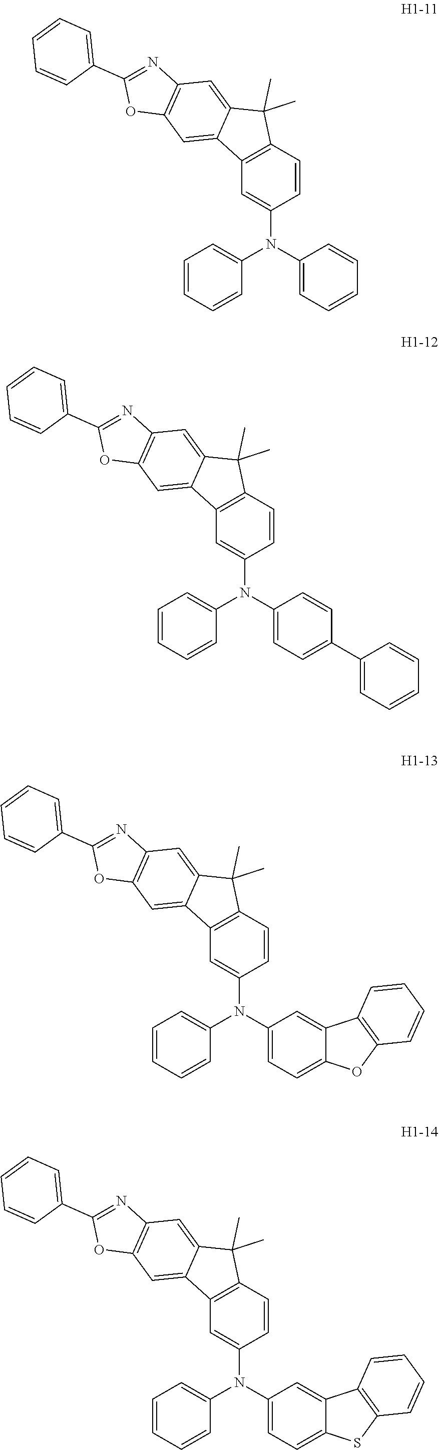 [SMILES: CC1(C)c2ccc(N(c3ccccc3)c3ccccc3)cc2-c2cc3oc(-c4ccccc4)nc3cc21 ; CC1(C)c2ccc(N(c3ccccc3)c3ccc(-c4ccccc4)cc3)cc2-c2cc3oc(-c4ccccc4)nc3cc21 ; CC1(C)c2ccc(N(c3ccccc3)c3ccc4oc5ccccc5c4c3)cc2-c2cc3oc(-c4ccccc4)nc3cc21 ; CC1(C)c2ccc(N(c3ccccc3)c3ccc4sc5ccccc5c4c3)cc2-c2cc3oc(-c4ccccc4)nc3cc21]

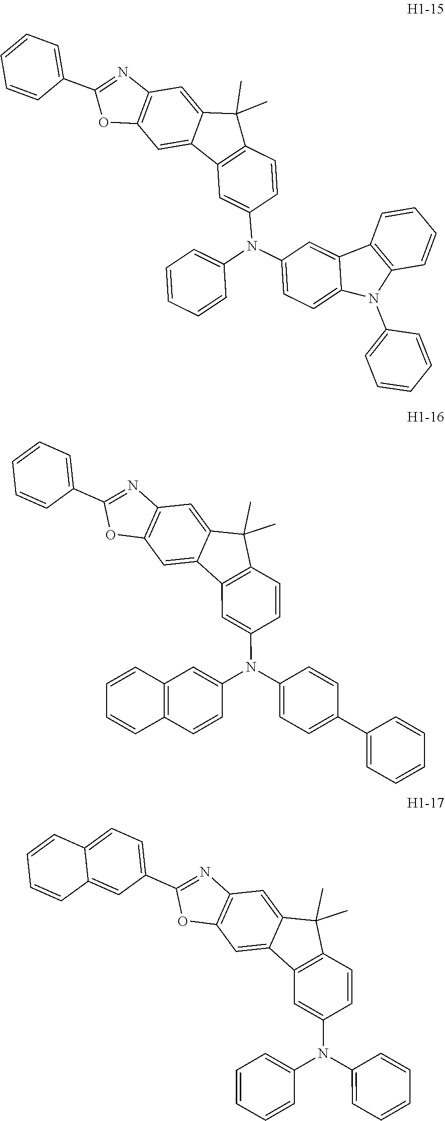 [SMILES: CC1(C)c2ccc(N(c3ccccc3)c3ccc4c(c3)c3ccccc3n4-c3ccccc3)cc2-c2cc3oc(-c4ccccc4)nc3cc21 ; CC1(C)c2ccc(N(c3ccc(-c4ccccc4)cc3)c3ccc4ccccc4c3)cc2-c2cc3oc(-c4ccccc4)nc3cc21 ; CC1(C)c2ccc(N(c3ccccc3)c3ccccc3)cc2-c2cc3oc(-c4ccc5ccccc5c4)nc3cc21]

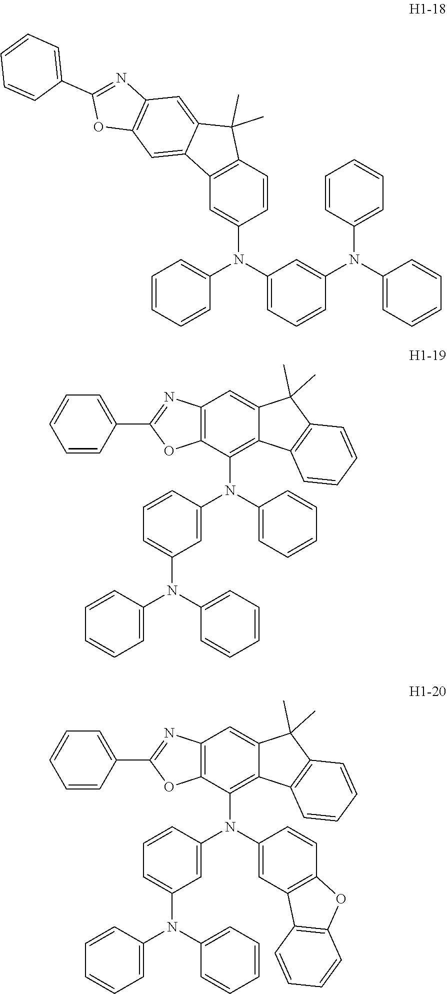

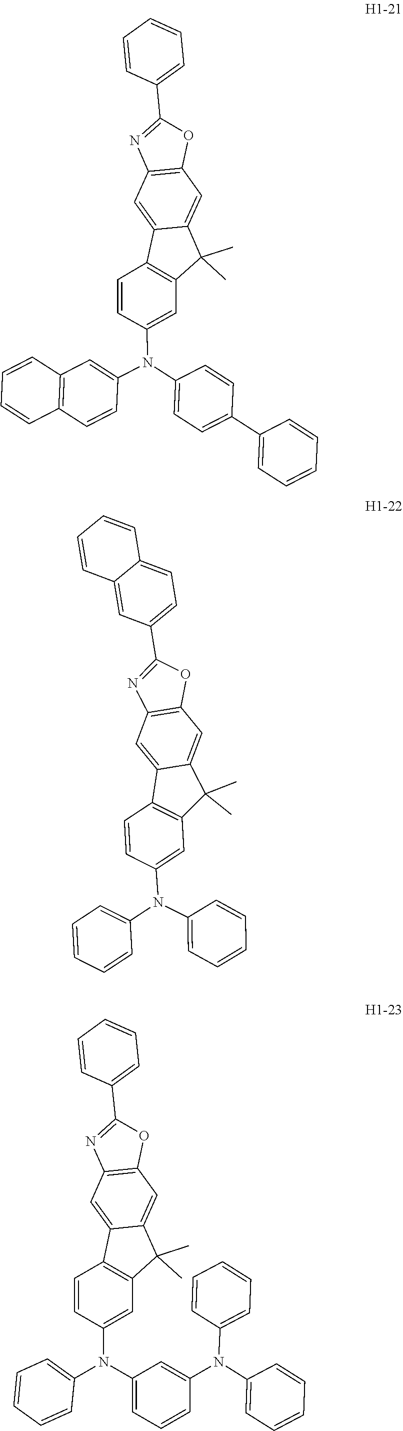 [SMILES: CC1(C)c2cc(N(c3ccc(-c4ccccc4)cc3)c3ccc4ccccc4c3)ccc2-c2cc3nc(-c4ccccc4)oc3cc21 ; CC1(C)c2cc(N(c3ccccc3)c3ccccc3)ccc2-c2cc3nc(-c4ccc5ccccc5c4)oc3cc21 ; CC1(C)c2cc(N(c3ccccc3)c3cccc(N(c4ccccc4)c4ccccc4)c3)ccc2-c2cc3nc(-c4ccccc4)oc3cc21]

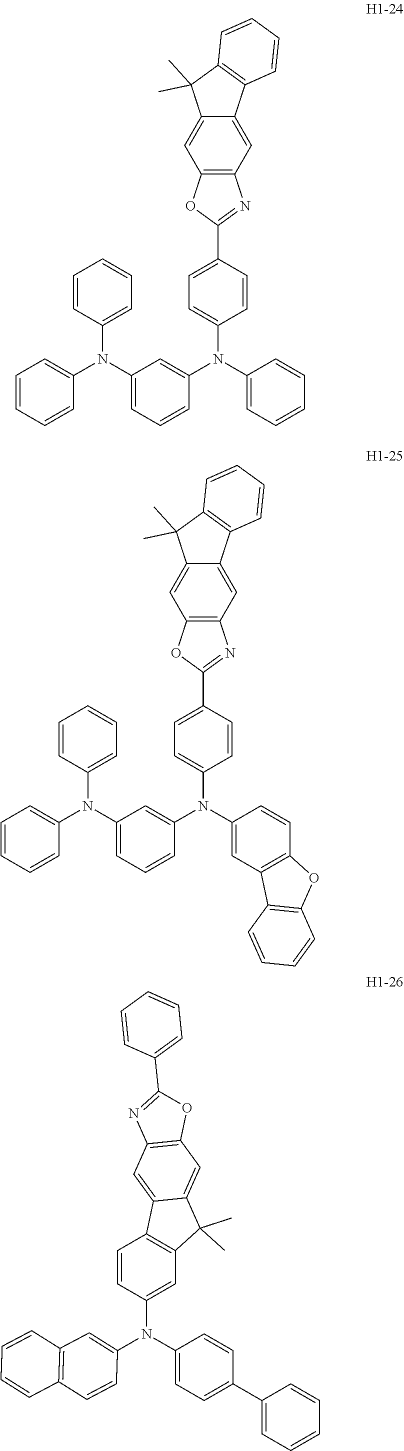 [SMILES: CC1(C)c2ccccc2-c2cc3nc(-c4ccc(N(c5ccccc5)c5cccc(N(c6ccccc6)c6ccccc6)c5)cc4)oc3cc21 ; CC1(C)c2ccccc2-c2cc3nc(-c4ccc(N(c5cccc(N(c6ccccc6)c6ccccc6)c5)c5ccc6oc7ccccc7c6c5)cc4)oc3cc21 ; CC1(C)c2cc(N(c3ccc(-c4ccccc4)cc3)c3ccc4ccccc4c3)ccc2-c2cc3nc(-c4ccccc4)oc3cc21]

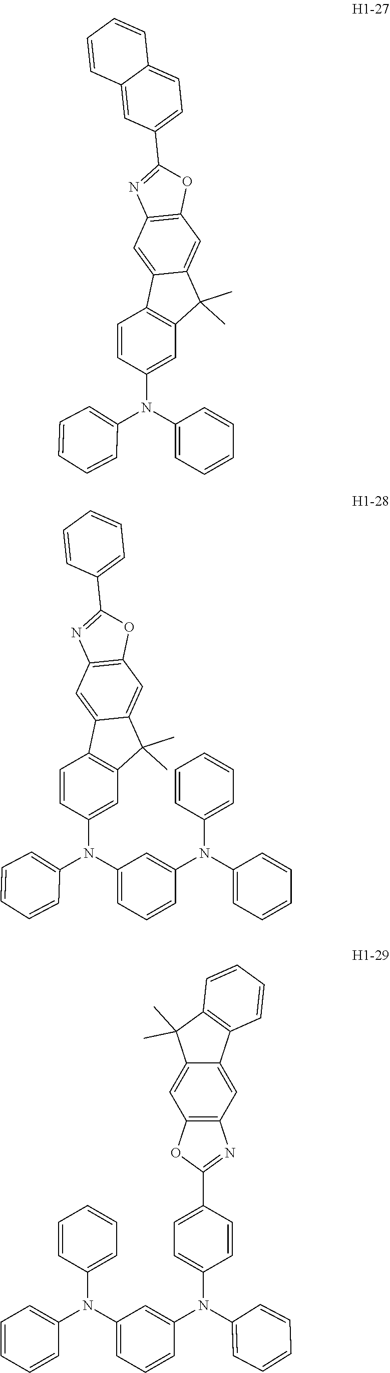

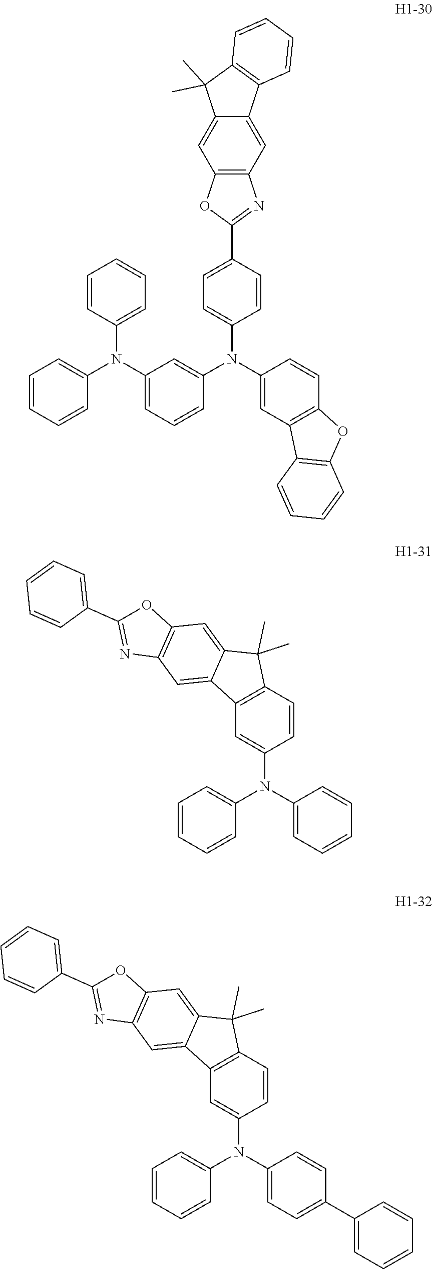 [SMILES: CC1(C)c2ccccc2-c2cc3nc(-c4ccc(N(c5cccc(N(c6ccccc6)c6ccccc6)c5)c5ccc6oc7ccccc7c6c5)cc4)oc3cc21 ; CC1(C)c2ccc(N(c3ccccc3)c3ccccc3)cc2-c2cc3nc(-c4ccccc4)oc3cc21 ; CC1(C)c2ccc(N(c3ccccc3)c3ccc(-c4ccccc4)cc3)cc2-c2cc3nc(-c4ccccc4)oc3cc21]

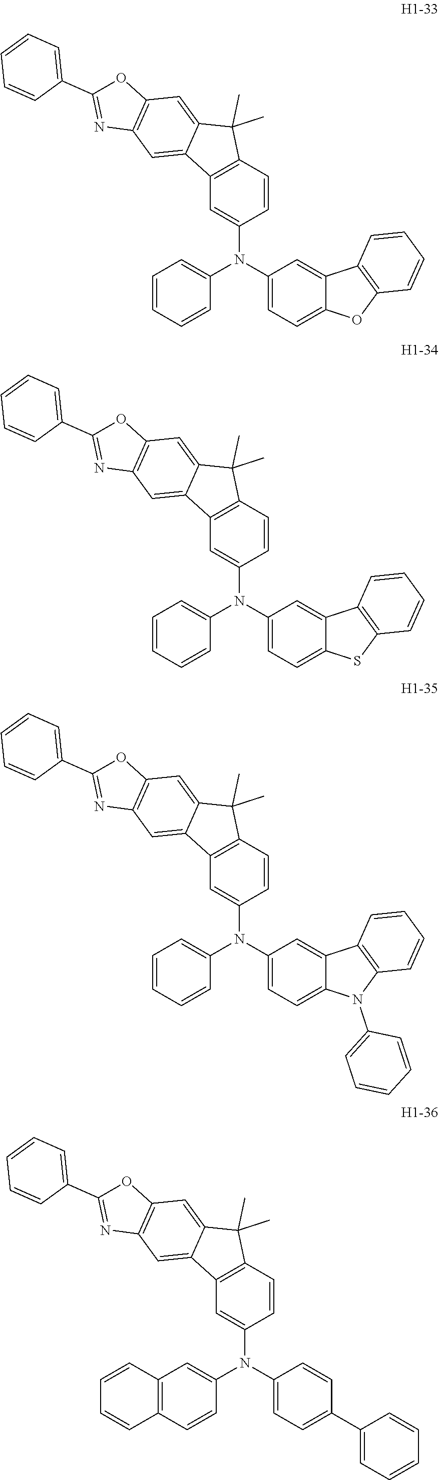 [SMILES: CC1(C)c2ccc(N(c3ccccc3)c3ccc4oc5ccccc5c4c3)cc2-c2cc3nc(-c4ccccc4)oc3cc21 ; CC1(C)c2ccc(N(c3ccccc3)c3ccc4sc5ccccc5c4c3)cc2-c2cc3nc(-c4ccccc4)oc3cc21 ; CC1(C)c2ccc(N(c3ccccc3)c3ccc4c(c3)c3ccccc3n4-c3ccccc3)cc2-c2cc3nc(-c4ccccc4)oc3cc21 ; CC1(C)c2ccc(N(c3ccc(-c4ccccc4)cc3)c3ccc4ccccc4c3)cc2-c2cc3nc(-c4ccccc4)oc3cc21]

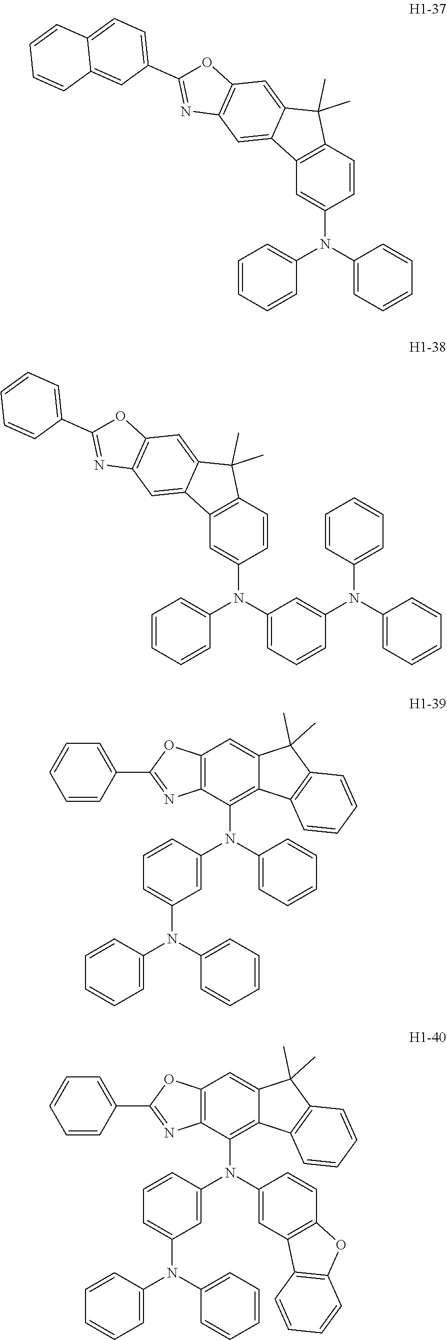 [SMILES: CC1(C)c2ccc(N(c3ccccc3)c3ccccc3)cc2-c2cc3nc(-c4ccc5ccccc5c4)oc3cc21 ; CC1(C)c2ccc(N(c3ccccc3)c3cccc(N(c4ccccc4)c4ccccc4)c3)cc2-c2cc3nc(-c4ccccc4)oc3cc21 ; CC1(C)c2ccccc2-c2c1cc1oc(-c3ccccc3)nc1c2N(c1ccccc1)c1cccc(N(c2ccccc2)c2ccccc2)c1 ; CC1(C)c2ccccc2-c2c1cc1oc(-c3ccccc3)nc1c2N(c1cccc(N(c2ccccc2)c2ccccc2)c1)c1ccc2oc3ccccc3c2c1]

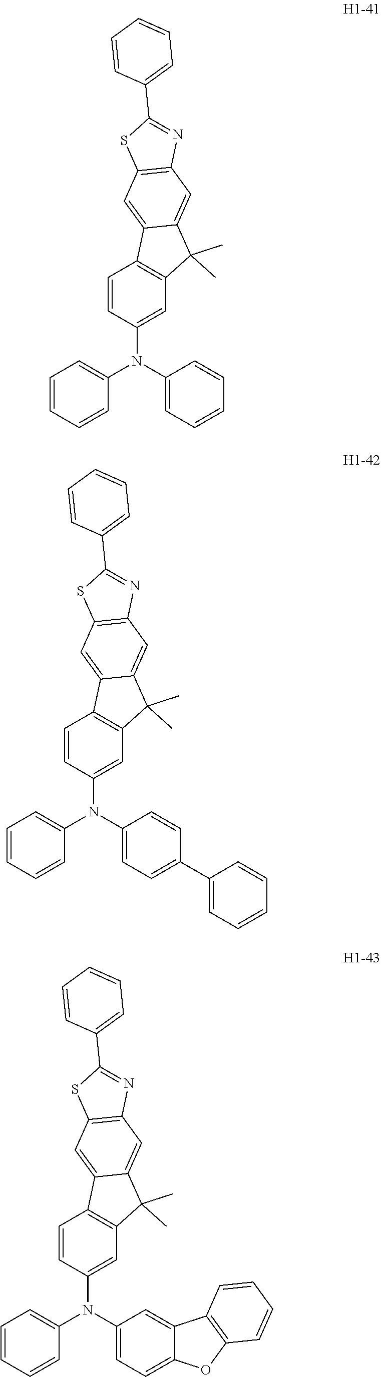

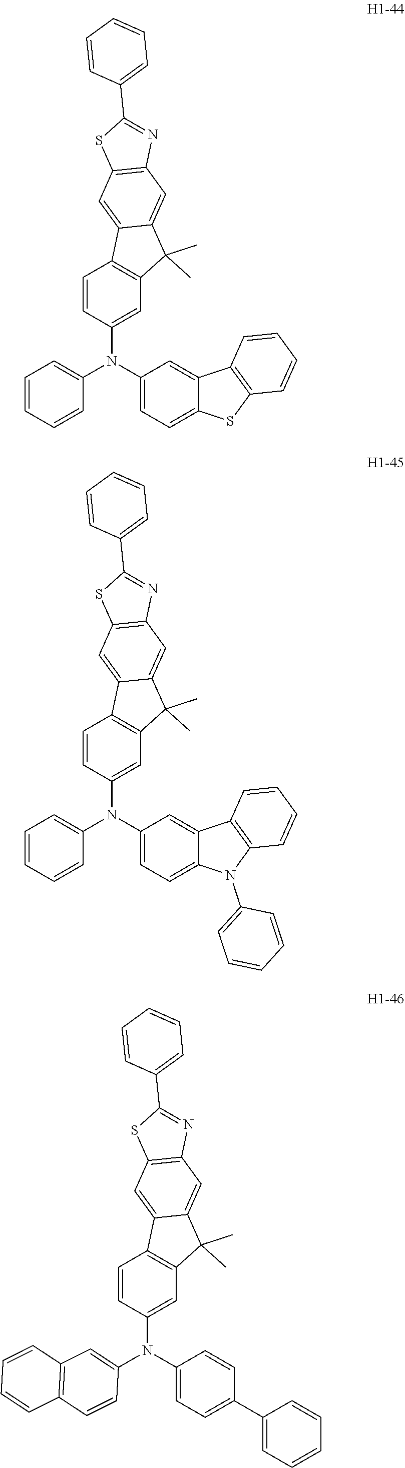 [SMILES: CC1(C)c2cc(N(c3ccccc3)c3ccc4sc5ccccc5c4c3)ccc2-c2cc3sc(-c4ccccc4)nc3cc21 ; CC1(C)c2cc(N(c3ccccc3)c3ccc4c(c3)c3ccccc3n4-c3ccccc3)ccc2-c2cc3sc(-c4ccccc4)nc3cc21 ; CC1(C)c2cc(N(c3ccc(-c4ccccc4)cc3)c3ccc4ccccc4c3)ccc2-c2cc3sc(-c4ccccc4)nc3cc21]

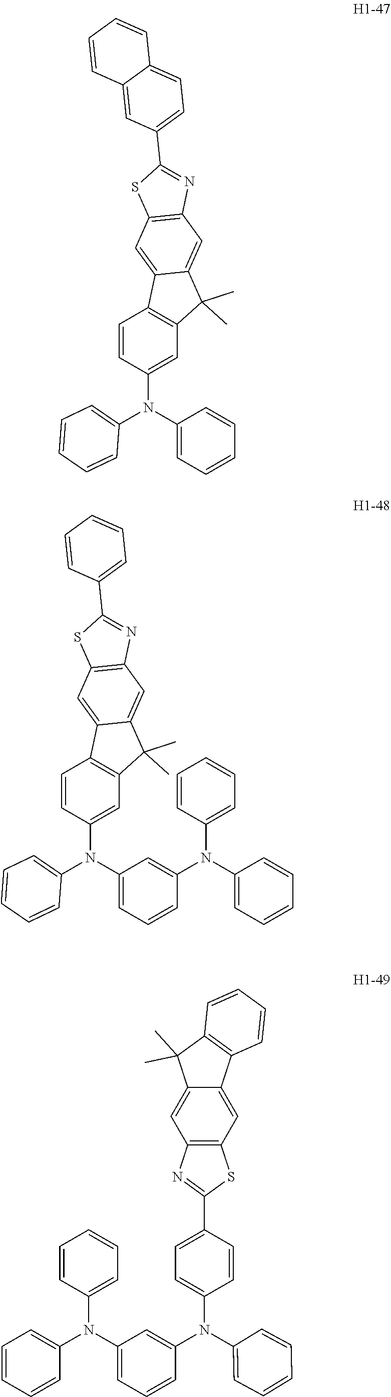

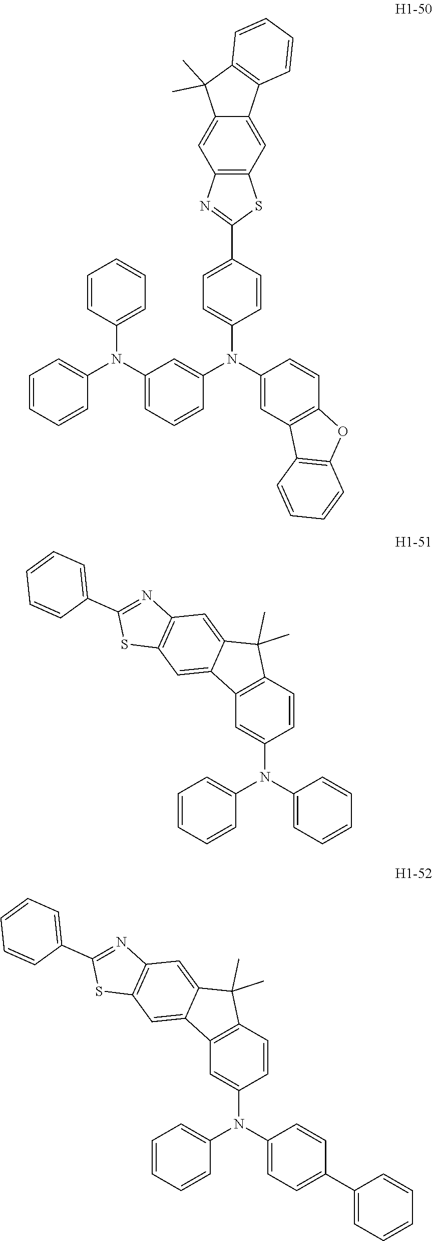 [SMILES: CC1(C)c2ccccc2-c2cc3sc(-c4ccc(N(c5cccc(N(c6ccccc6)c6ccccc6)c5)c5ccc6oc7ccccc7c6c5)cc4)nc3cc21 ; CC1(C)c2ccc(N(c3ccccc3)c3ccccc3)cc2-c2cc3sc(-c4ccccc4)nc3cc21 ; CC1(C)c2ccc(N(c3ccccc3)c3ccc(-c4ccccc4)cc3)cc2-c2cc3sc(-c4ccccc4)nc3cc21]

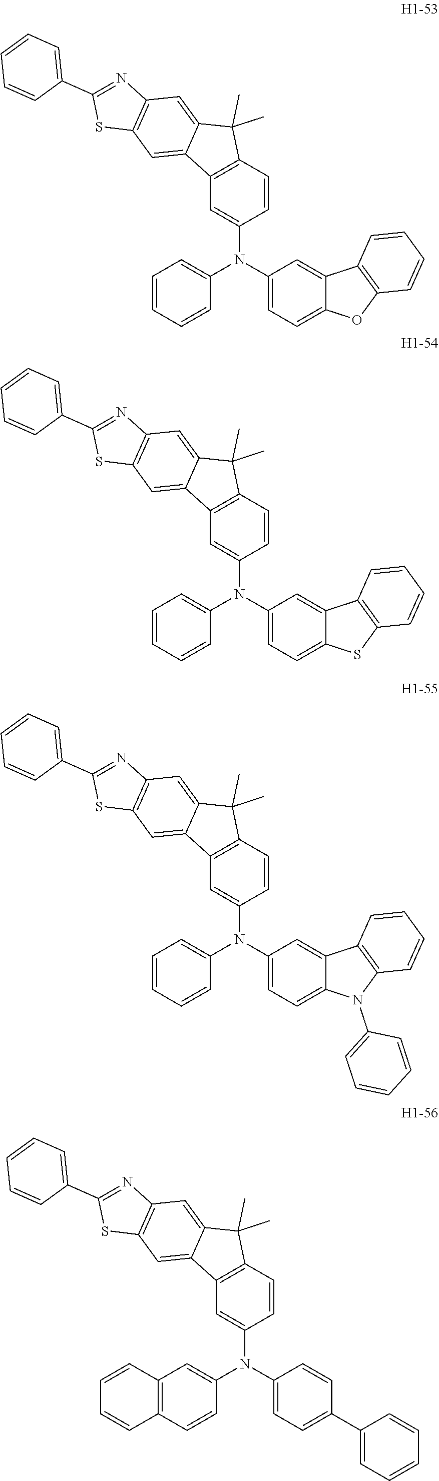 [SMILES: CC1(C)c2ccc(N(c3ccccc3)c3ccc4oc5ccccc5c4c3)cc2-c2cc3sc(-c4ccccc4)nc3cc21 ; CC1(C)c2ccc(N(c3ccccc3)c3ccc4sc5ccccc5c4c3)cc2-c2cc3sc(-c4ccccc4)nc3cc21 ; CC1(C)c2ccc(N(c3ccccc3)c3ccc4c(c3)c3ccccc3n4-c3ccccc3)cc2-c2cc3sc(-c4ccccc4)nc3cc21 ; CC1(C)c2ccc(N(c3ccc(-c4ccccc4)cc3)c3ccc4ccccc4c3)cc2-c2cc3sc(-c4ccccc4)nc3cc21]

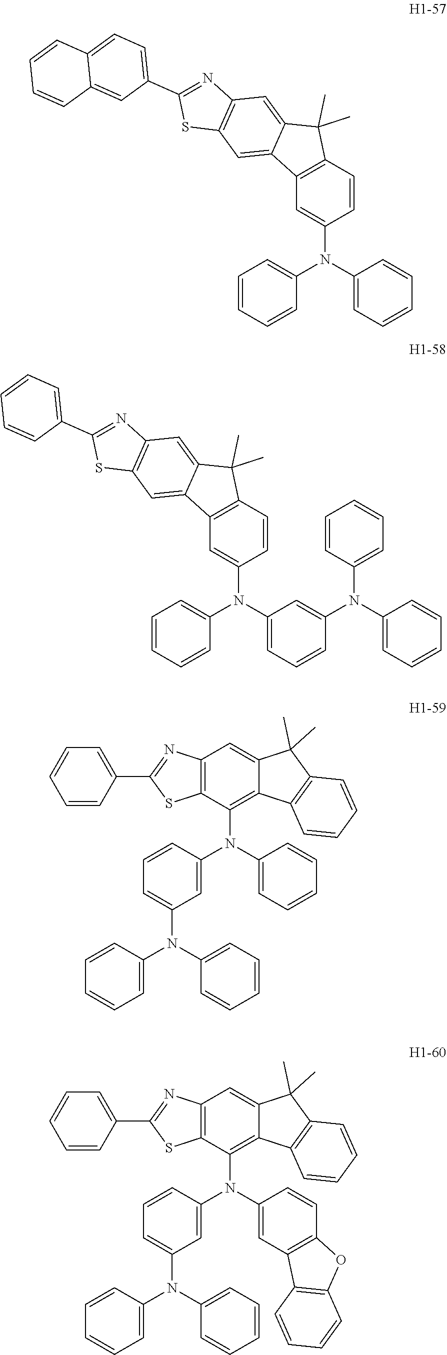 [SMILES: CC1(C)c2ccc(N(c3ccccc3)c3ccccc3)cc2-c2cc3sc(-c4ccc5ccccc5c4)nc3cc21 ; CC1(C)c2ccc(N(c3ccccc3)c3cccc(N(c4ccccc4)c4ccccc4)c3)cc2-c2cc3sc(-c4ccccc4)nc3cc21 ; CC1(C)c2ccccc2-c2c1cc1nc(-c3ccccc3)sc1c2N(c1ccccc1)c1cccc(N(c2ccccc2)c2ccccc2)c1 ; CC1(C)c2ccccc2-c2c1cc1nc(-c3ccccc3)sc1c2N(c1cccc(N(c2ccccc2)c2ccccc2)c1)c1ccc2oc3ccccc3c2c1]

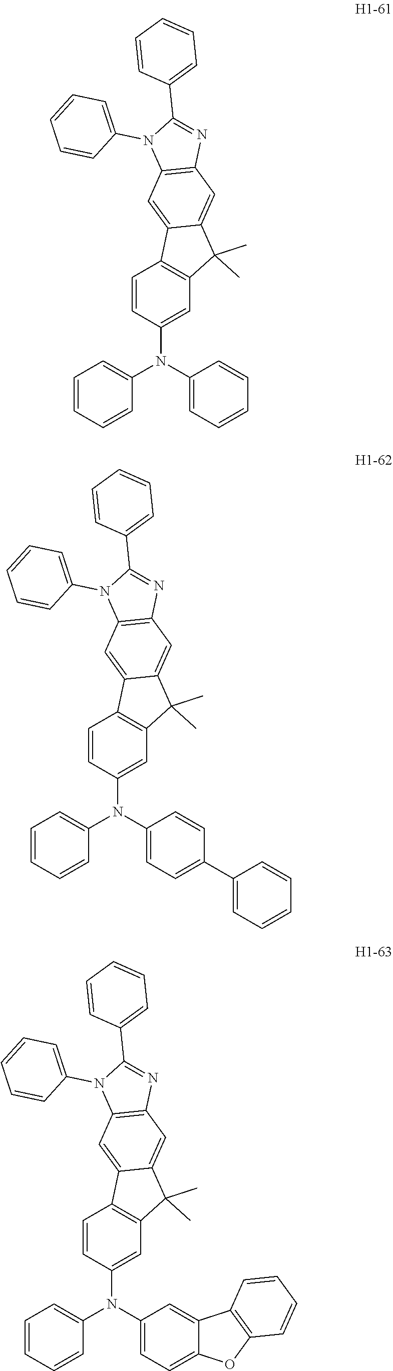

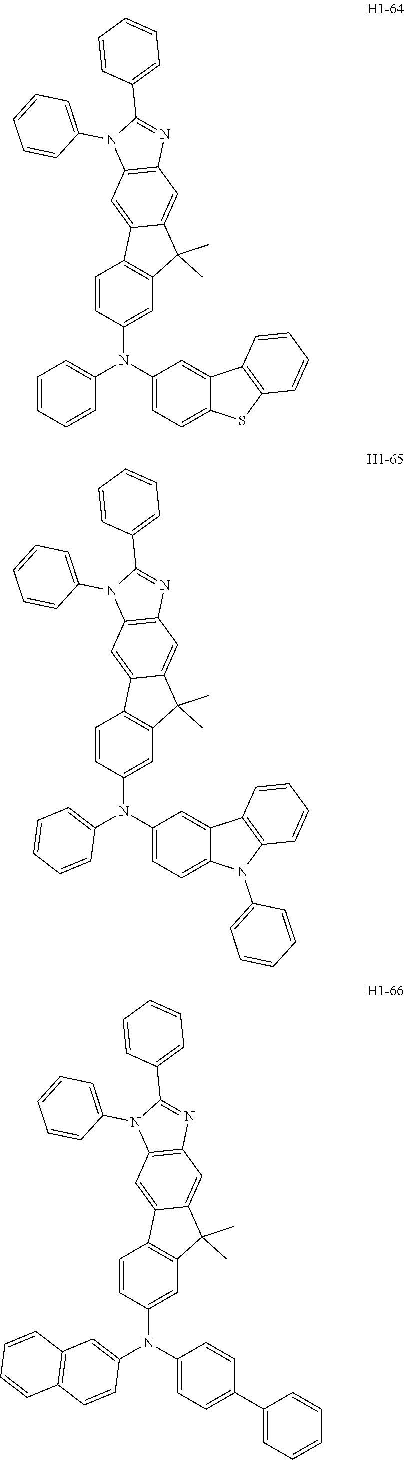 [SMILES: CC1(C)c2cc(N(c3ccccc3)c3ccc4sc5ccccc5c4c3)ccc2-c2cc3c(cc21)nc(-c1ccccc1)n3-c1ccccc1 ; CC1(C)c2cc(N(c3ccccc3)c3ccc4c(c3)c3ccccc3n4-c3ccccc3)ccc2-c2cc3c(cc21)nc(-c1ccccc1)n3-c1ccccc1 ; CC1(C)c2cc(N(c3ccc(-c4ccccc4)cc3)c3ccc4ccccc4c3)ccc2-c2cc3c(cc21)nc(-c1ccccc1)n3-c1ccccc1]

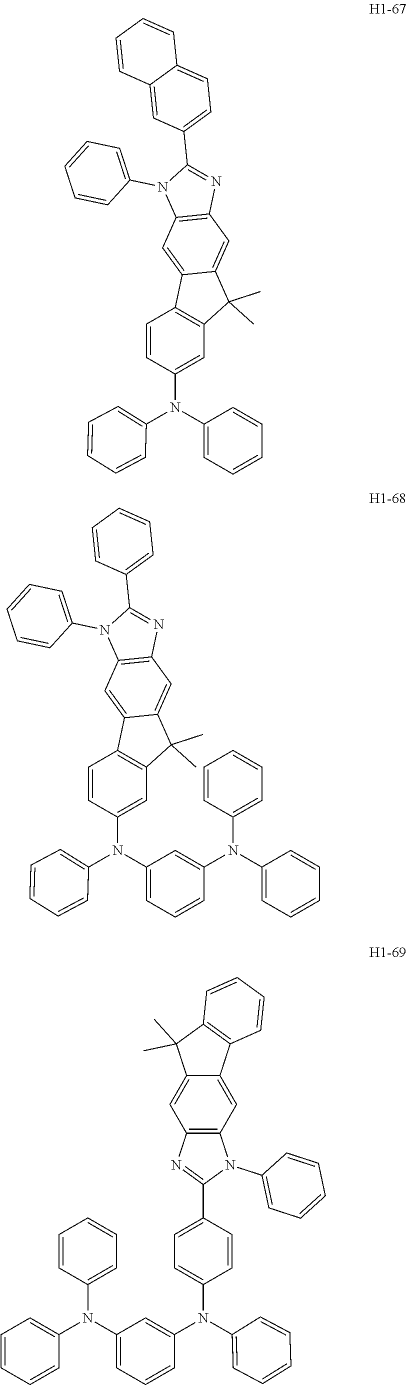

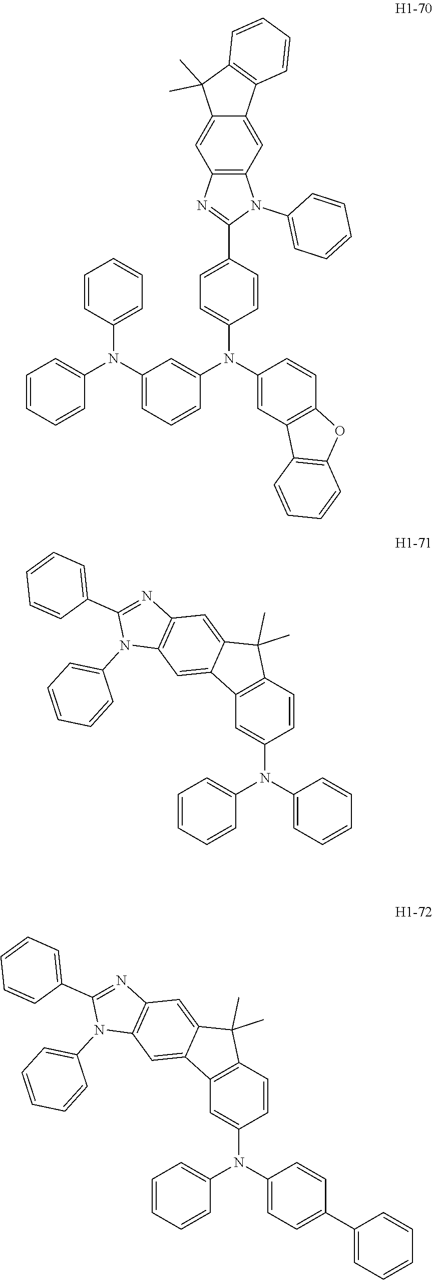 [SMILES: CC1(C)c2ccccc2-c2cc3c(cc21)nc(-c1ccc(N(c2cccc(N(c4ccccc4)c4ccccc4)c2)c2ccc4oc5ccccc5c4c2)cc1)n3-c1ccccc1 ; CC1(C)c2ccc(N(c3ccccc3)c3ccccc3)cc2-c2cc3c(cc21)nc(-c1ccccc1)n3-c1ccccc1 ; CC1(C)c2ccc(N(c3ccccc3)c3ccc(-c4ccccc4)cc3)cc2-c2cc3c(cc21)nc(-c1ccccc1)n3-c1ccccc1]

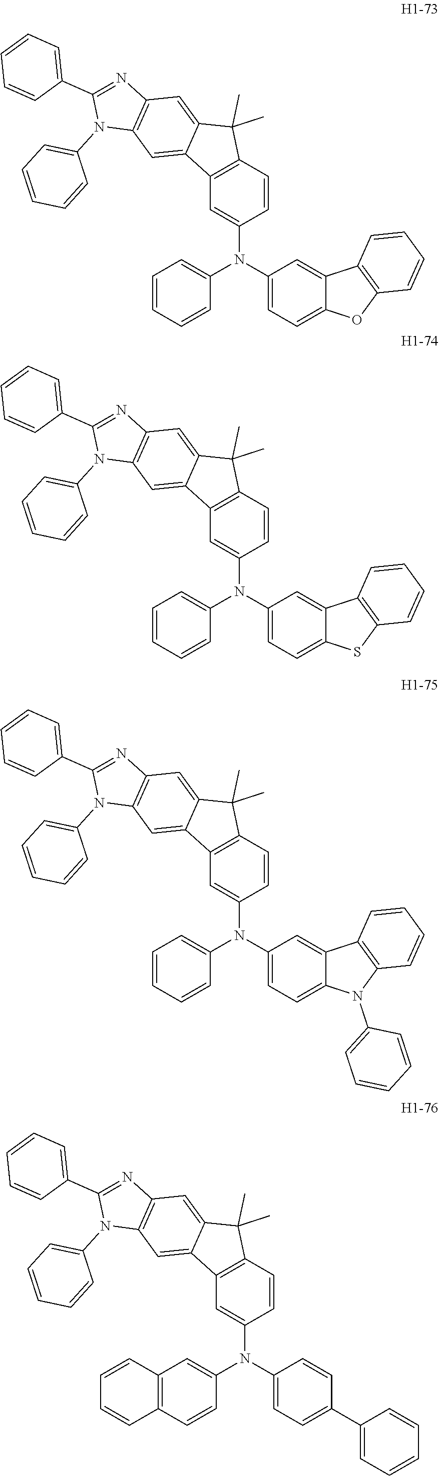 [SMILES: CC1(C)c2ccc(N(c3ccccc3)c3ccc4oc5ccccc5c4c3)cc2-c2cc3c(cc21)nc(-c1ccccc1)n3-c1ccccc1 ; CC1(C)c2ccc(N(c3ccccc3)c3ccc4sc5ccccc5c4c3)cc2-c2cc3c(cc21)nc(-c1ccccc1)n3-c1ccccc1 ; CC1(C)c2ccc(N(c3ccccc3)c3ccc4c(c3)c3ccccc3n4-c3ccccc3)cc2-c2cc3c(cc21)nc(-c1ccccc1)n3-c1ccccc1 ; CC1(C)c2ccc(N(c3ccc(-c4ccccc4)cc3)c3ccc4ccccc4c3)cc2-c2cc3c(cc21)nc(-c1ccccc1)n3-c1ccccc1]

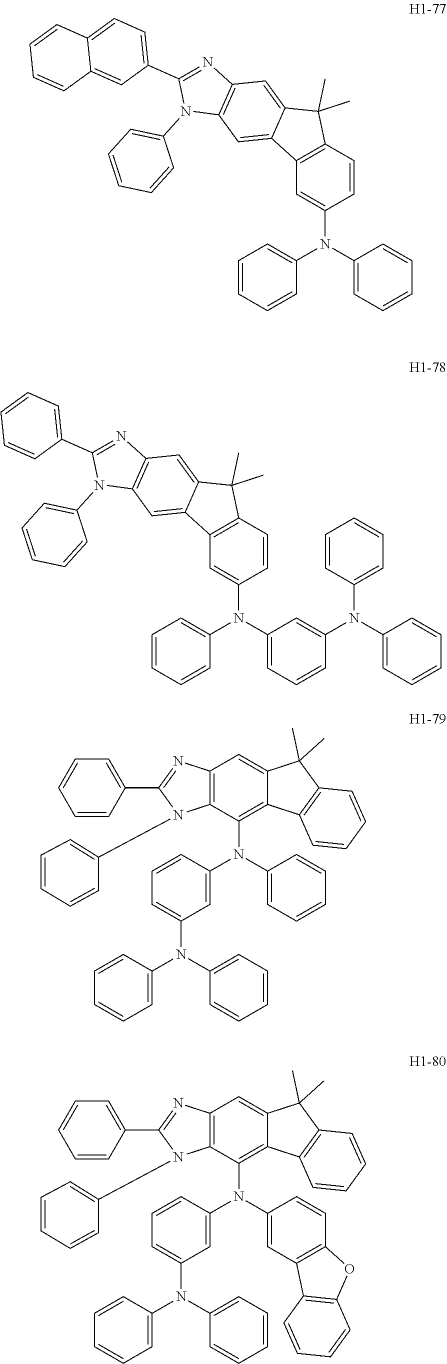

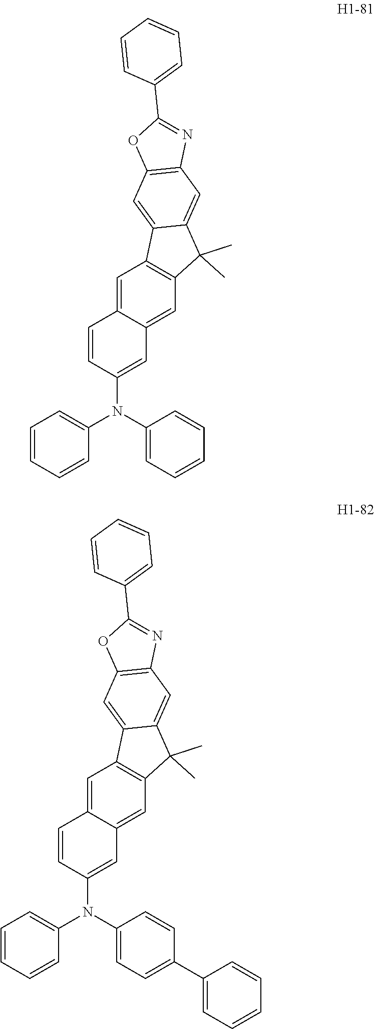 [SMILES: CC1(C)c2cc3cc(N(c4ccccc4)c4ccccc4)ccc3cc2-c2cc3oc(-c4ccccc4)nc3cc21 ; CC1(C)c2cc3cc(N(c4ccccc4)c4ccc(-c5ccccc5)cc4)ccc3cc2-c2cc3oc(-c4ccccc4)nc3cc21]

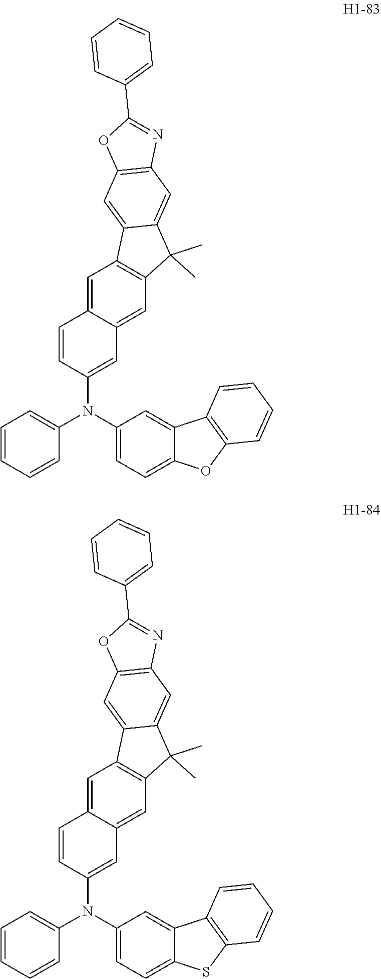 [SMILES: CC1(C)c2cc3cc(N(c4ccccc4)c4ccc5oc6ccccc6c5c4)ccc3cc2-c2cc3oc(-c4ccccc4)nc3cc21 ; CC1(C)c2cc3cc(N(c4ccccc4)c4ccc5sc6ccccc6c5c4)ccc3cc2-c2cc3oc(-c4ccccc4)nc3cc21]

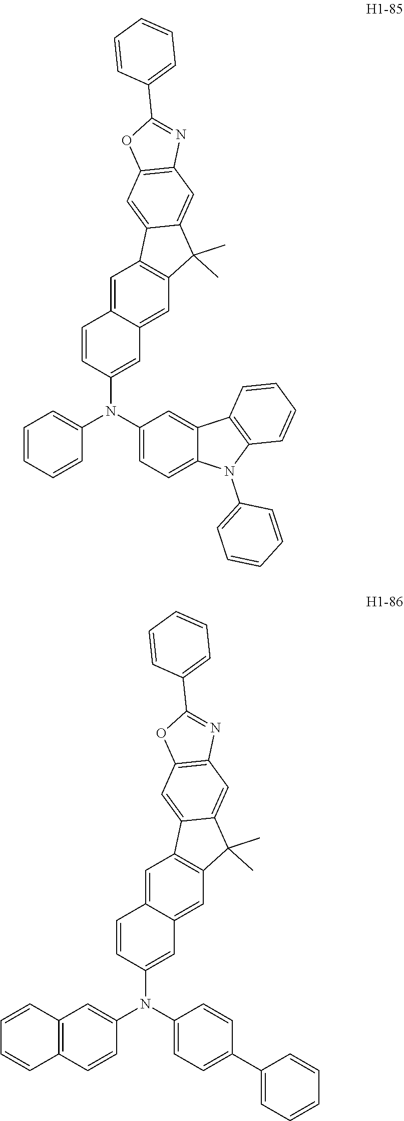 [SMILES: CC1(C)c2cc3cc(N(c4ccccc4)c4ccc5c(c4)c4ccccc4n5-c4ccccc4)ccc3cc2-c2cc3oc(-c4ccccc4)nc3cc21 ; CC1(C)c2cc3cc(N(c4ccc(-c5ccccc5)cc4)c4ccc5ccccc5c4)ccc3cc2-c2cc3oc(-c4ccccc4)nc3cc21]

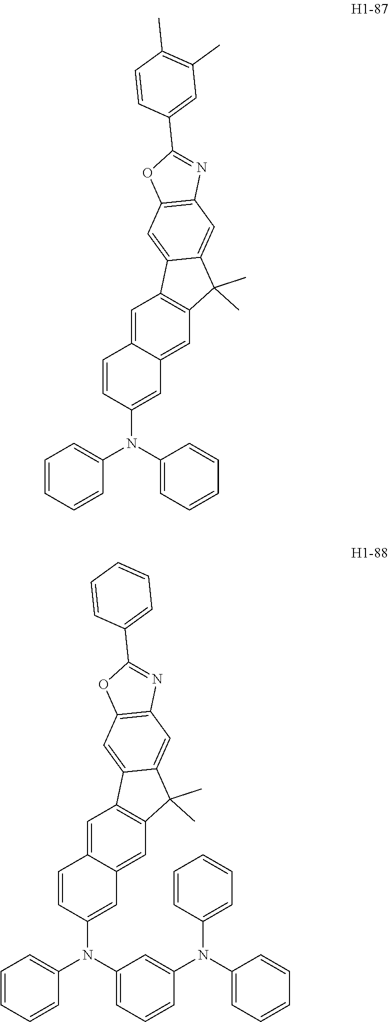 [SMILES: Cc1ccc(-c2nc3cc4c(cc3o2)-c2cc3ccc(N(c5ccccc5)c5ccccc5)cc3cc2C4(C)C)cc1C ; CC1(C)c2cc3cc(N(c4ccccc4)c4cccc(N(c5ccccc5)c5ccccc5)c4)ccc3cc2-c2cc3oc(-c4ccccc4)nc3cc21]

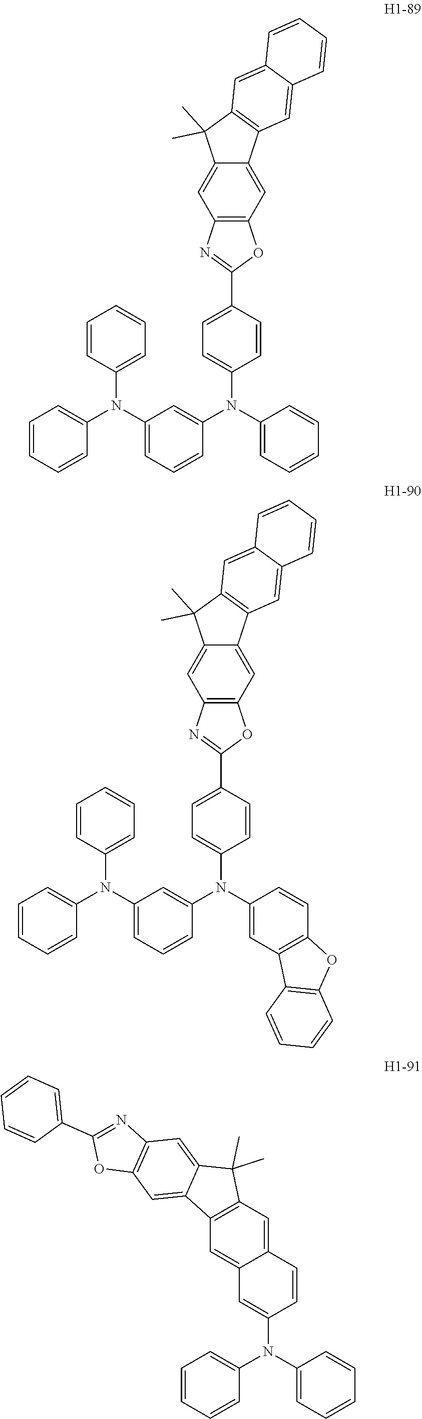

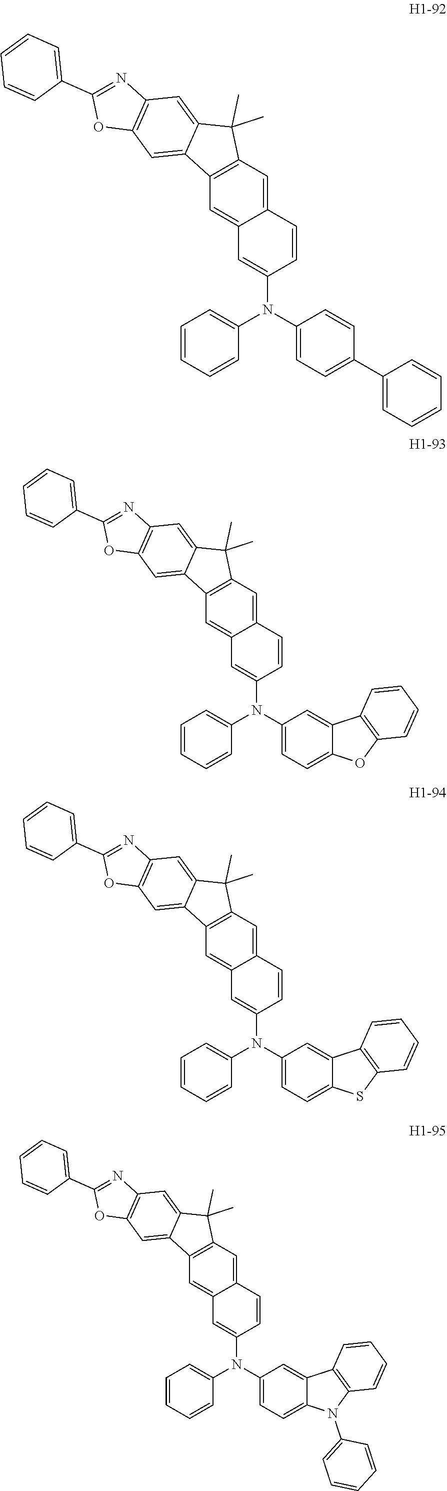 [SMILES: CC1(C)c2cc3ccc(N(c4ccccc4)c4ccc(-c5ccccc5)cc4)cc3cc2-c2cc3oc(-c4ccccc4)nc3cc21 ; CC1(C)c2cc3ccc(N(c4ccccc4)c4ccc5oc6ccccc6c5c4)cc3cc2-c2cc3oc(-c4ccccc4)nc3cc21 ; CC1(C)c2cc3ccc(N(c4ccccc4)c4ccc5sc6ccccc6c5c4)cc3cc2-c2cc3oc(-c4ccccc4)nc3cc21 ; CC1(C)c2cc3ccc(N(c4ccccc4)c4ccc5c(c4)c4ccccc4n5-c4ccccc4)cc3cc2-c2cc3oc(-c4ccccc4)nc3cc21]

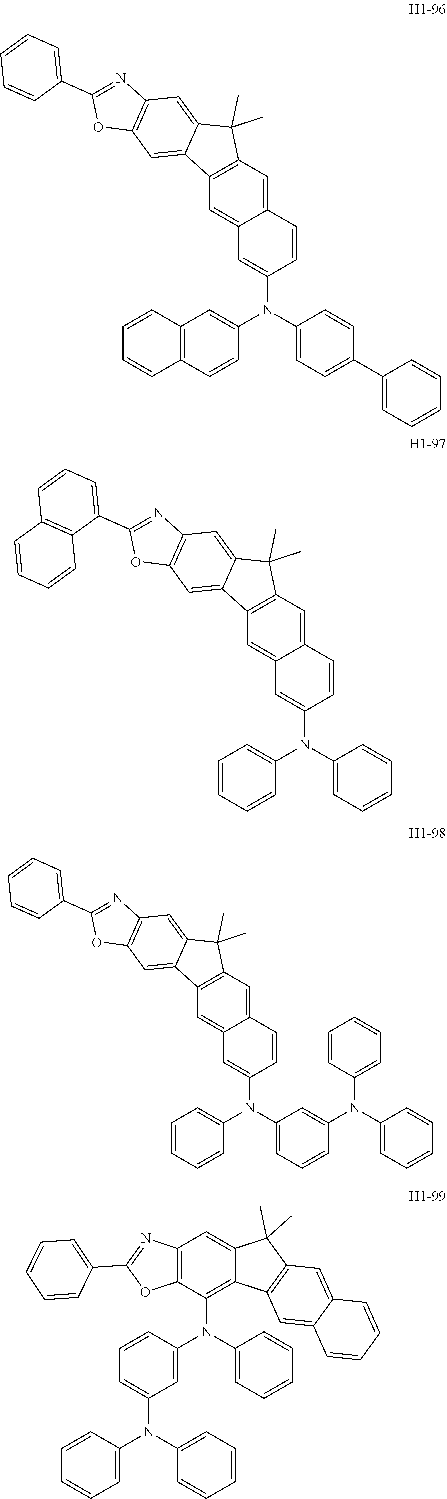 [SMILES: CC1(C)c2cc3ccc(N(c4ccc(-c5ccccc5)cc4)c4ccc5ccccc5c4)cc3cc2-c2cc3oc(-c4ccccc4)nc3cc21 ; CC1(C)c2cc3ccc(N(c4ccccc4)c4ccccc4)cc3cc2-c2cc3oc(-c4cccc5ccccc45)nc3cc21 ; CC1(C)c2cc3ccc(N(c4ccccc4)c4cccc(N(c5ccccc5)c5ccccc5)c4)cc3cc2-c2cc3oc(-c4ccccc4)nc3cc21 ; CC1(C)c2cc3ccccc3cc2-c2c1cc1nc(-c3ccccc3)oc1c2N(c1ccccc1)c1cccc(N(c2ccccc2)c2ccccc2)c1]

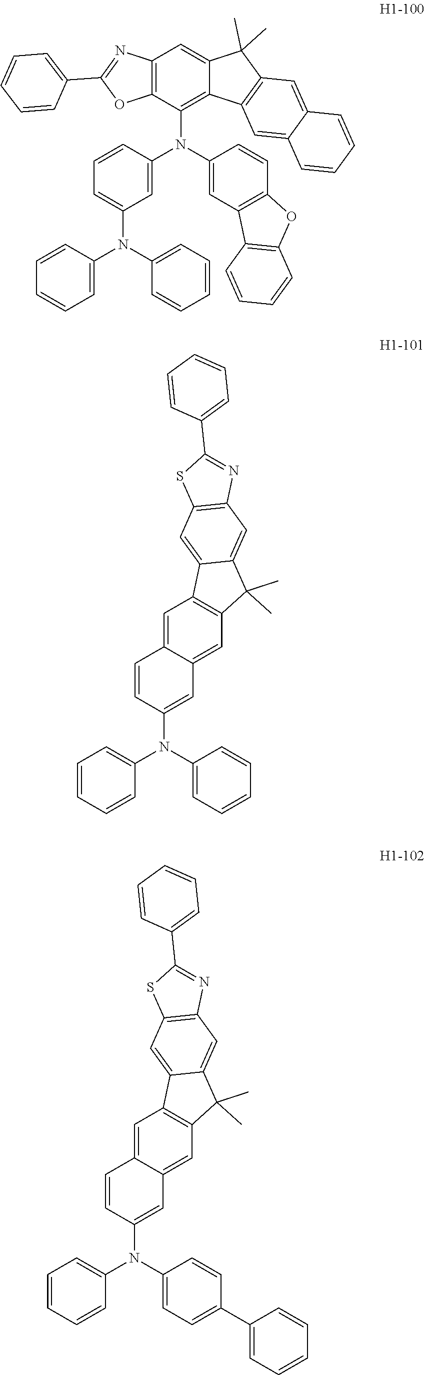

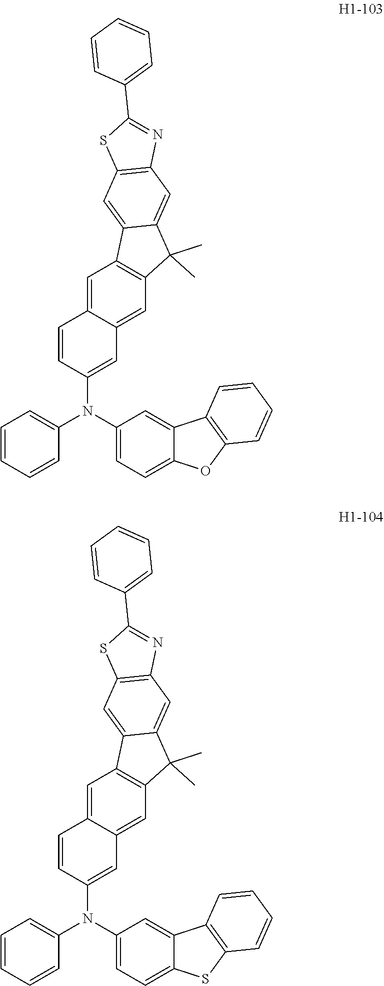 [SMILES: CC1(C)c2cc3cc(N(c4ccccc4)c4ccc5oc6ccccc6c5c4)ccc3cc2-c2cc3sc(-c4ccccc4)nc3cc21 ; CC1(C)c2cc3cc(N(c4ccccc4)c4ccc5sc6ccccc6c5c4)ccc3cc2-c2cc3sc(-c4ccccc4)nc3cc21]

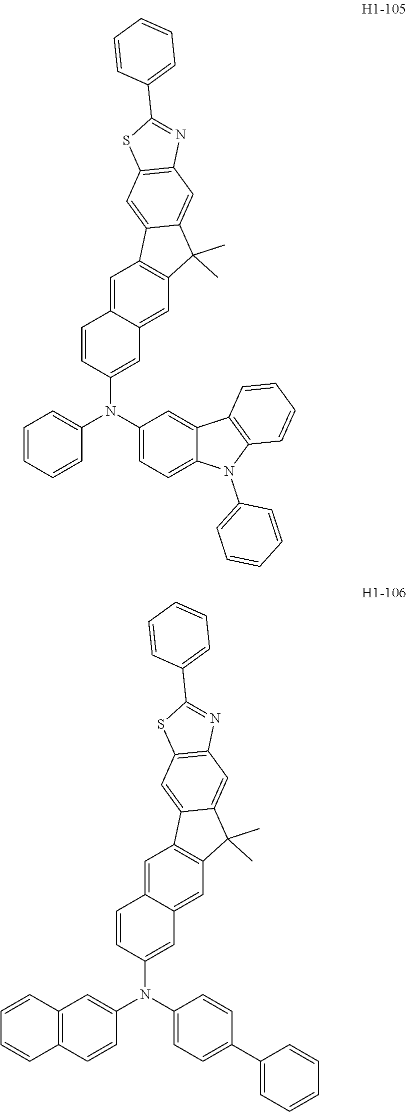 [SMILES: CC1(C)c2cc3cc(N(c4ccccc4)c4ccc5c(c4)c4ccccc4n5-c4ccccc4)ccc3cc2-c2cc3sc(-c4ccccc4)nc3cc21 ; CC1(C)c2cc3cc(N(c4ccc(-c5ccccc5)cc4)c4ccc5ccccc5c4)ccc3cc2-c2cc3sc(-c4ccccc4)nc3cc21]

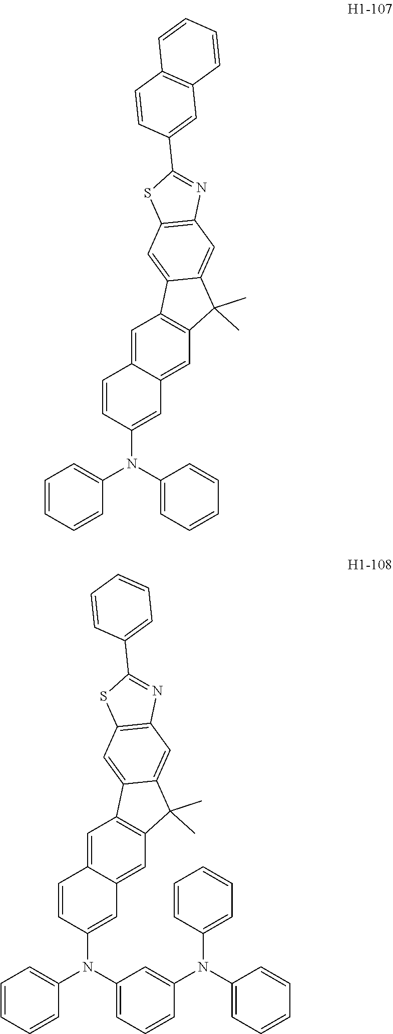 [SMILES: CC1(C)c2cc3cc(N(c4ccccc4)c4ccccc4)ccc3cc2-c2cc3sc(-c4ccc5ccccc5c4)nc3cc21 ; CC1(C)c2cc3cc(N(c4ccccc4)c4cccc(N(c5ccccc5)c5ccccc5)c4)ccc3cc2-c2cc3sc(-c4ccccc4)nc3cc21]

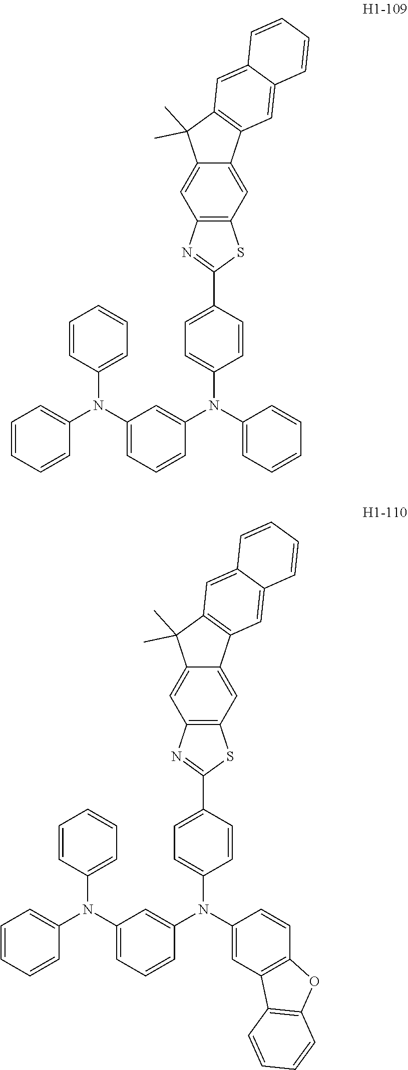 [SMILES: CC1(C)c2cc3ccccc3cc2-c2cc3sc(-c4ccc(N(c5ccccc5)c5cccc(N(c6ccccc6)c6ccccc6)c5)cc4)nc3cc21 ; CC1(C)c2cc3ccccc3cc2-c2cc3sc(-c4ccc(N(c5cccc(N(c6ccccc6)c6ccccc6)c5)c5ccc6oc7ccccc7c6c5)cc4)nc3cc21]

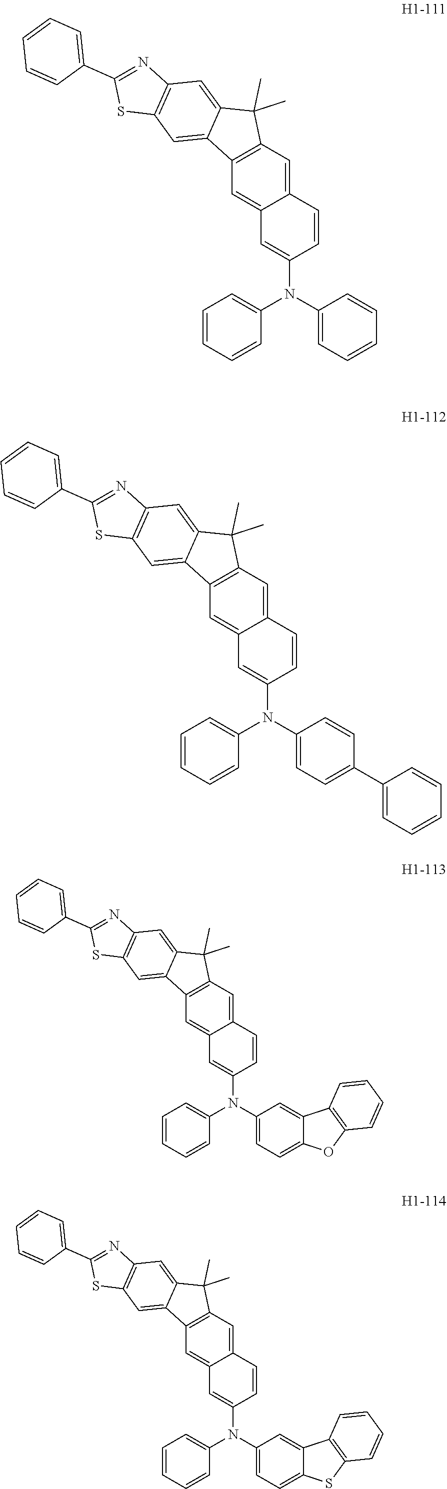 [SMILES: CC1(C)c2cc3ccc(N(c4ccccc4)c4ccccc4)cc3cc2-c2cc3sc(-c4ccccc4)nc3cc21 ; CC1(C)c2cc3ccc(N(c4ccccc4)c4ccc(-c5ccccc5)cc4)cc3cc2-c2cc3sc(-c4ccccc4)nc3cc21 ; CC1(C)c2cc3ccc(N(c4ccccc4)c4ccc5oc6ccccc6c5c4)cc3cc2-c2cc3sc(-c4ccccc4)nc3cc21 ; CC1(C)c2cc3ccc(N(c4ccccc4)c4ccc5sc6ccccc6c5c4)cc3cc2-c2cc3sc(-c4ccccc4)nc3cc21]

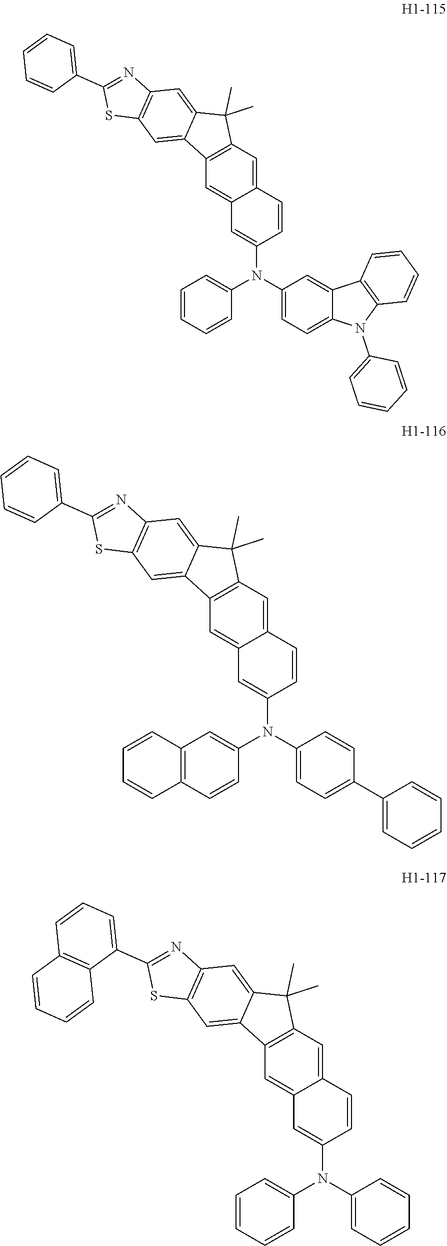 [SMILES: CC1(C)c2cc3ccc(N(c4ccccc4)c4ccc5c(c4)c4ccccc4n5-c4ccccc4)cc3cc2-c2cc3sc(-c4ccccc4)nc3cc21 ; CC1(C)c2cc3ccc(N(c4ccc(-c5ccccc5)cc4)c4ccc5ccccc5c4)cc3cc2-c2cc3sc(-c4ccccc4)nc3cc21 ; CC1(C)c2cc3ccc(N(c4ccccc4)c4ccccc4)cc3cc2-c2cc3sc(-c4cccc5ccccc45)nc3cc21]

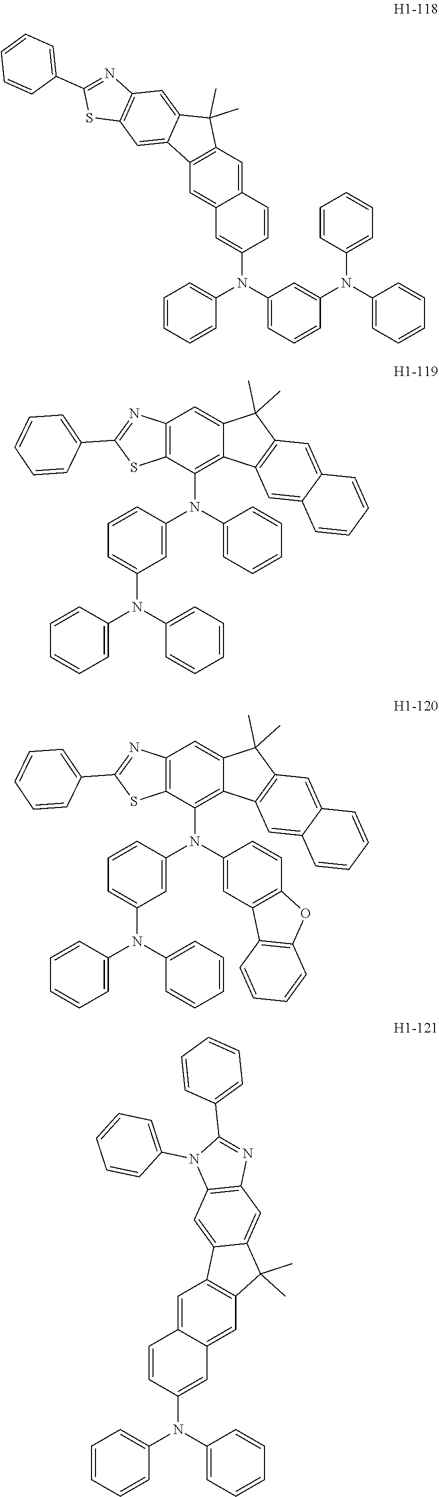

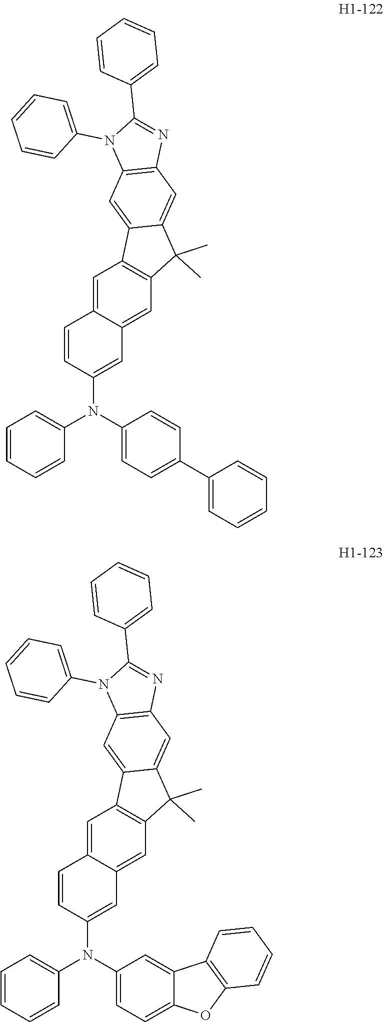 [SMILES: CC1(C)c2cc3cc(N(c4ccccc4)c4ccc(-c5ccccc5)cc4)ccc3cc2-c2cc3c(cc21)nc(-c1ccccc1)n3-c1ccccc1 ; CC1(C)c2cc3cc(N(c4ccccc4)c4ccc5oc6ccccc6c5c4)ccc3cc2-c2cc3c(cc21)nc(-c1ccccc1)n3-c1ccccc1]

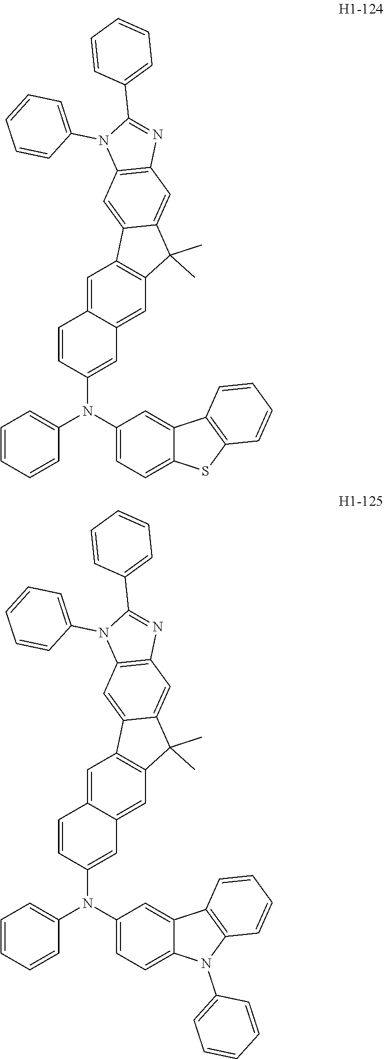

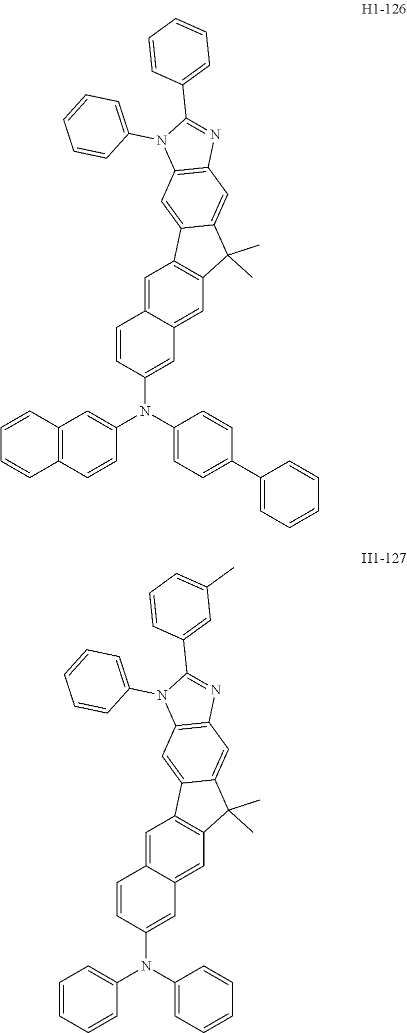 [SMILES: CC1(C)c2cc3cc(N(c4ccc(-c5ccccc5)cc4)c4ccc5ccccc5c4)ccc3cc2-c2cc3c(cc21)nc(-c1ccccc1)n3-c1ccccc1 ; Cc1cccc(-c2nc3cc4c(cc3n2-c2ccccc2)-c2cc3ccc(N(c5ccccc5)c5ccccc5)cc3cc2C4(C)C)c1]

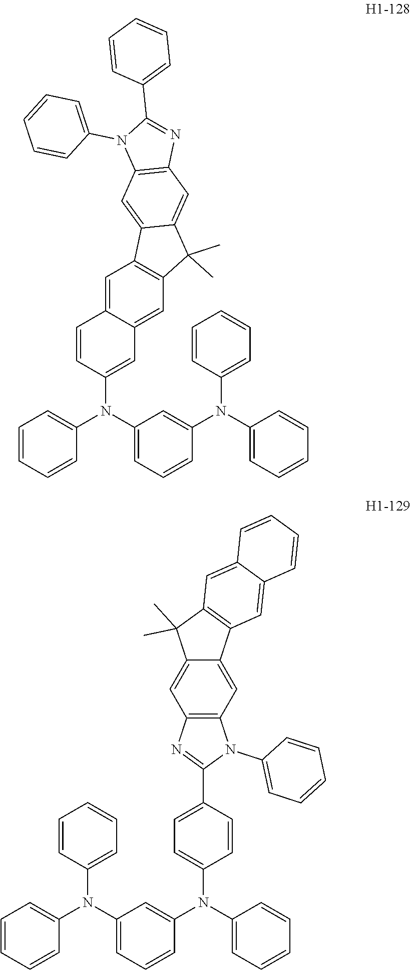

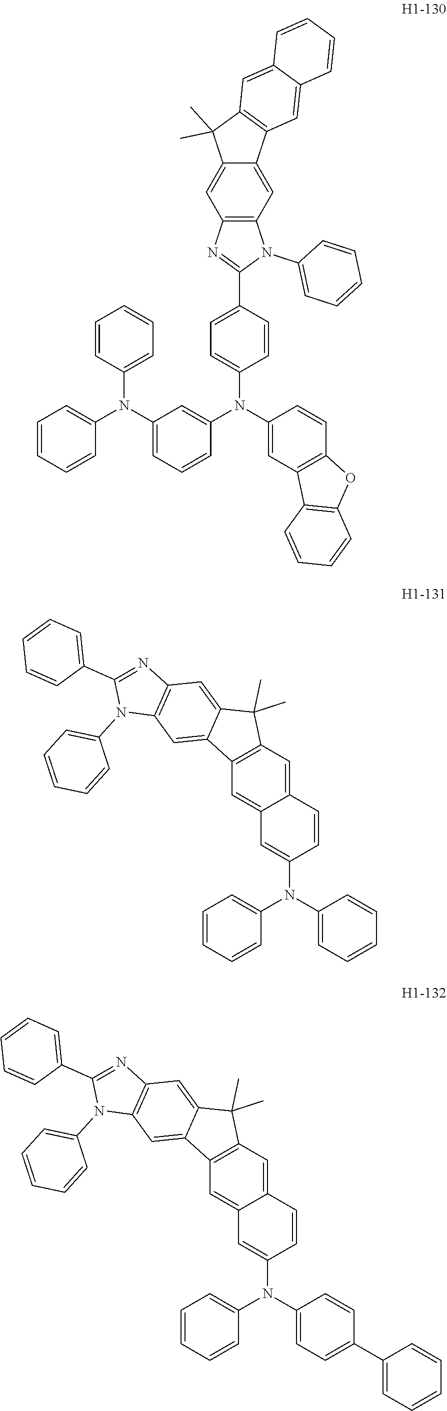 [SMILES: CC1(C)c2cc3ccccc3cc2-c2cc3c(cc21)nc(-c1ccc(N(c2cccc(N(c4ccccc4)c4ccccc4)c2)c2ccc4oc5ccccc5c4c2)cc1)n3-c1ccccc1 ; CC1(C)c2cc3ccc(N(c4ccccc4)c4ccccc4)cc3cc2-c2cc3c(cc21)nc(-c1ccccc1)n3-c1ccccc1 ; CC1(C)c2cc3ccc(N(c4ccccc4)c4ccc(-c5ccccc5)cc4)cc3cc2-c2cc3c(cc21)nc(-c1ccccc1)n3-c1ccccc1]

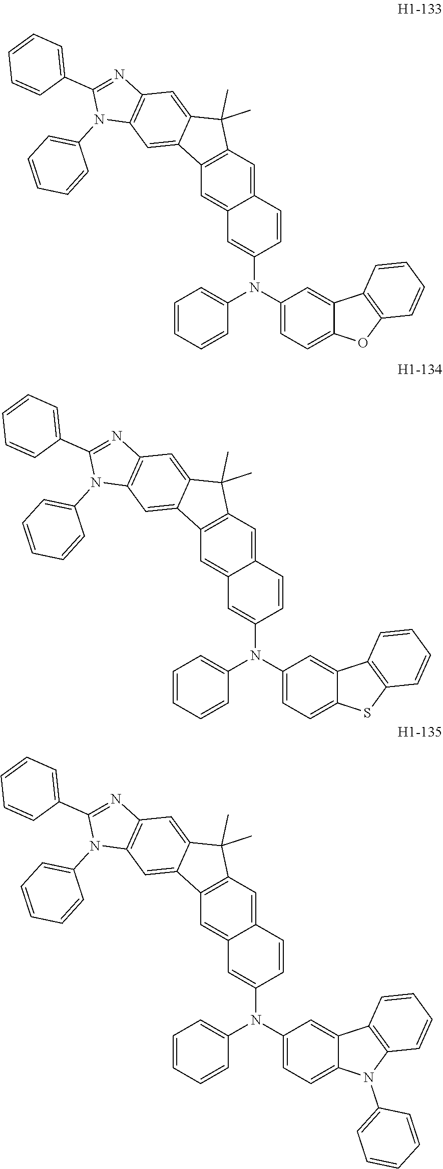 [SMILES: CC1(C)c2cc3ccc(N(c4ccccc4)c4ccc5oc6ccccc6c5c4)cc3cc2-c2cc3c(cc21)nc(-c1ccccc1)n3-c1ccccc1 ; CC1(C)c2cc3ccc(N(c4ccccc4)c4ccc5sc6ccccc6c5c4)cc3cc2-c2cc3c(cc21)nc(-c1ccccc1)n3-c1ccccc1 ; CC1(C)c2cc3ccc(N(c4ccccc4)c4ccc5c(c4)c4ccccc4n5-c4ccccc4)cc3cc2-c2cc3c(cc21)nc(-c1ccccc1)n3-c1ccccc1]

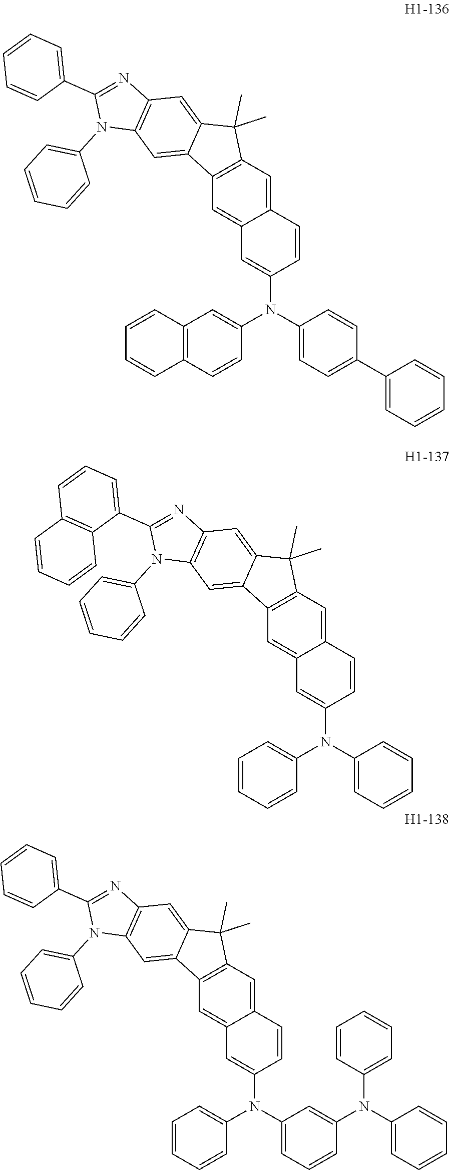 [SMILES: CC1(C)c2cc3ccc(N(c4ccc(-c5ccccc5)cc4)c4ccc5ccccc5c4)cc3cc2-c2cc3c(cc21)nc(-c1ccccc1)n3-c1ccccc1 ; CC1(C)c2cc3ccc(N(c4ccccc4)c4ccccc4)cc3cc2-c2cc3c(cc21)nc(-c1cccc2ccccc12)n3-c1ccccc1 ; CC1(C)c2cc3ccc(N(c4ccccc4)c4cccc(N(c5ccccc5)c5ccccc5)c4)cc3cc2-c2cc3c(cc21)nc(-c1ccccc1)n3-c1ccccc1]

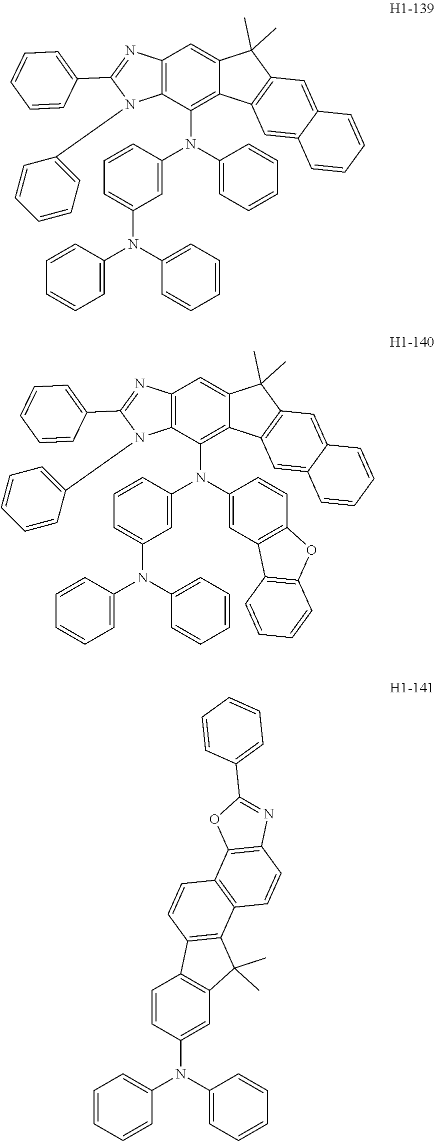 [SMILES: CC1(C)c2cc3ccccc3cc2-c2c1cc1nc(-c3ccccc3)n(-c3ccccc3)c1c2N(c1ccccc1)c1cccc(N(c2ccccc2)c2ccccc2)c1 ; CC1(C)c2cc3ccccc3cc2-c2c1cc1nc(-c3ccccc3)n(-c3ccccc3)c1c2N(c1cccc(N(c2ccccc2)c2ccccc2)c1)c1ccc2oc3ccccc3c2c1 ; CC1(C)c2cc(N(c3ccccc3)c3ccccc3)ccc2-c2ccc3c(ccc4nc(-c5ccccc5)oc43)c21]

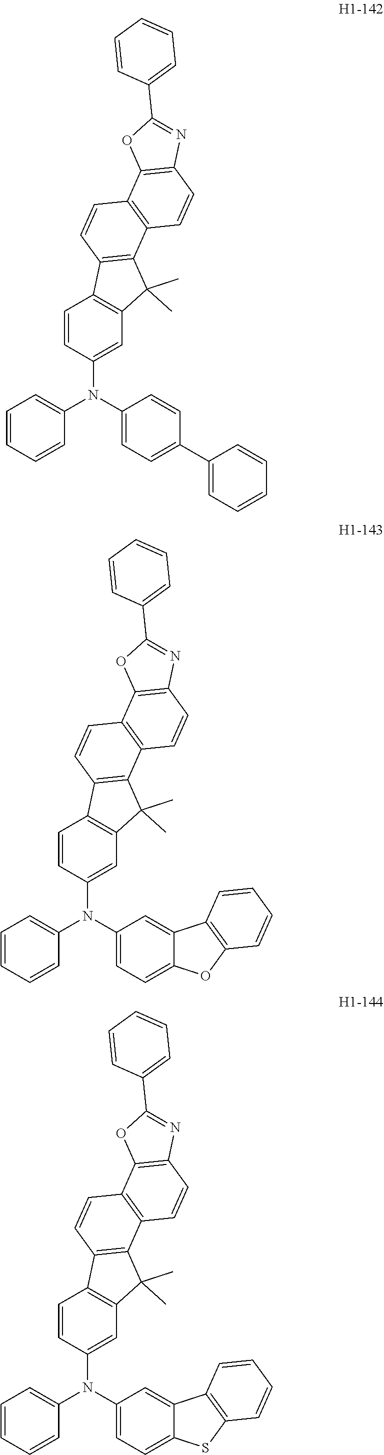

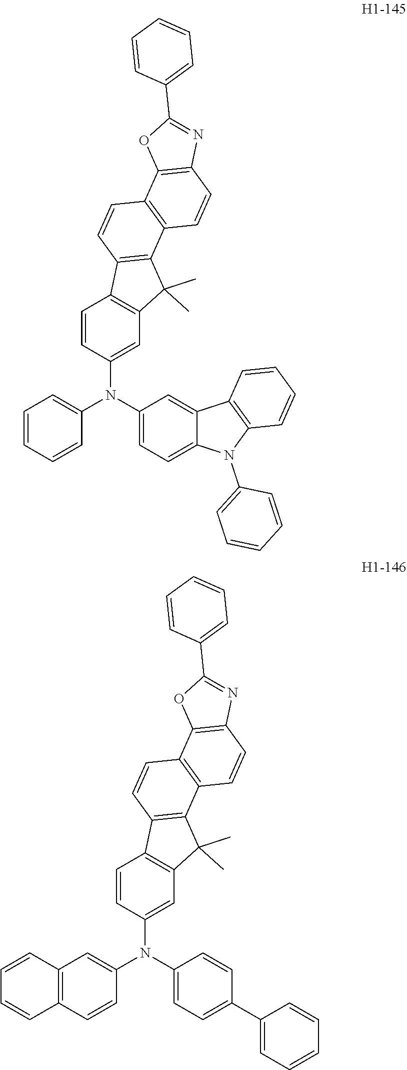 [SMILES: CC1(C)c2cc(N(c3ccccc3)c3ccc4c(c3)c3ccccc3n4-c3ccccc3)ccc2-c2ccc3c(ccc4nc(-c5ccccc5)oc43)c21 ; CC1(C)c2cc(N(c3ccc(-c4ccccc4)cc3)c3ccc4ccccc4c3)ccc2-c2ccc3c(ccc4nc(-c5ccccc5)oc43)c21]

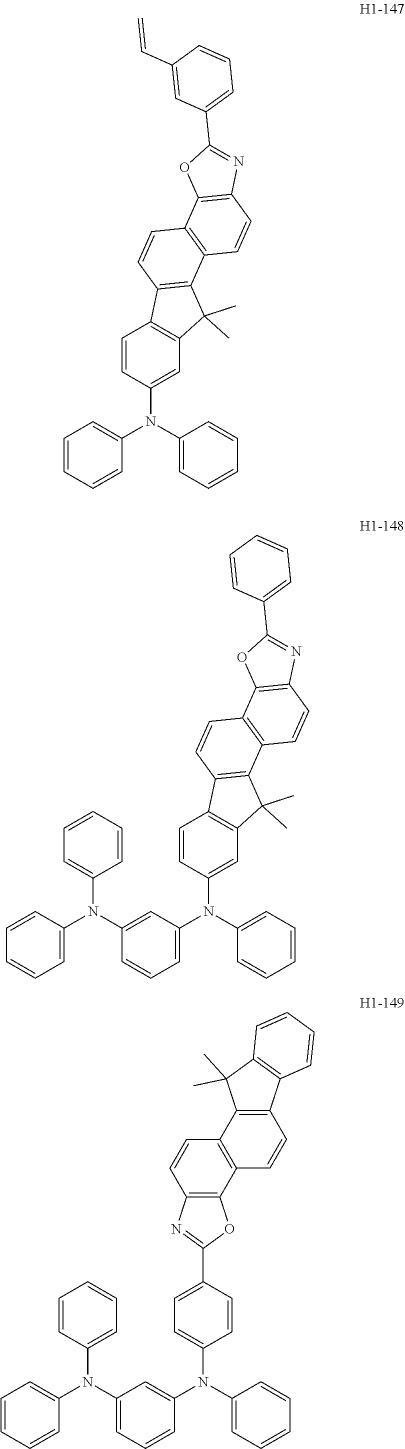

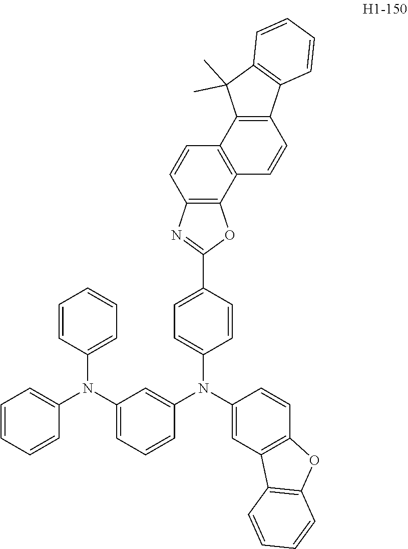 [SMILES: CC1(C)c2ccccc2-c2ccc3c(ccc4nc(-c5ccc(N(c6cccc(N(c7ccccc7)c7ccccc7)c6)c6ccc7oc8ccccc8c7c6)cc5)oc43)c21]

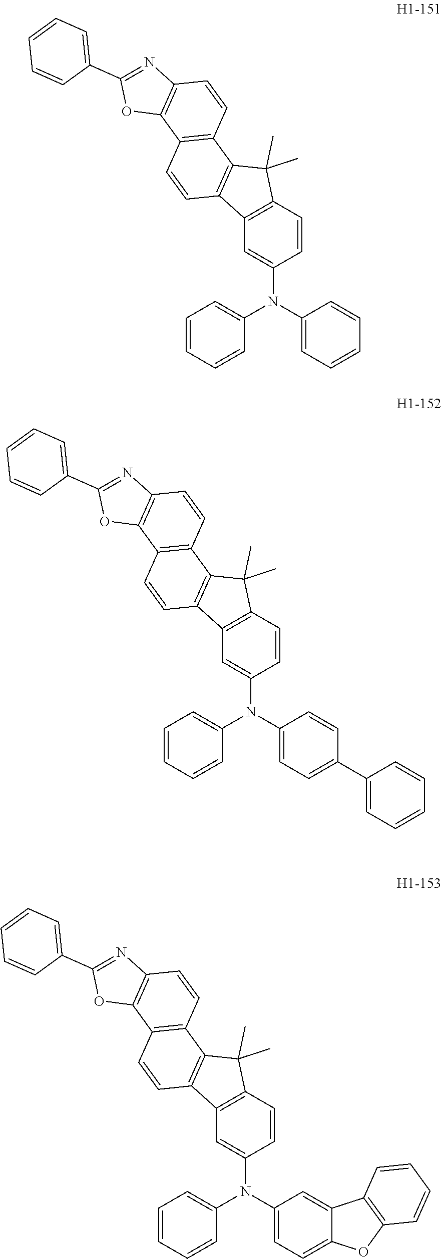 [SMILES: CC1(C)c2ccc(N(c3ccccc3)c3ccccc3)cc2-c2ccc3c(ccc4nc(-c5ccccc5)oc43)c21 ; CC1(C)c2ccc(N(c3ccccc3)c3ccc(-c4ccccc4)cc3)cc2-c2ccc3c(ccc4nc(-c5ccccc5)oc43)c21 ; CC1(C)c2ccc(N(c3ccccc3)c3ccc4oc5ccccc5c4c3)cc2-c2ccc3c(ccc4nc(-c5ccccc5)oc43)c21]

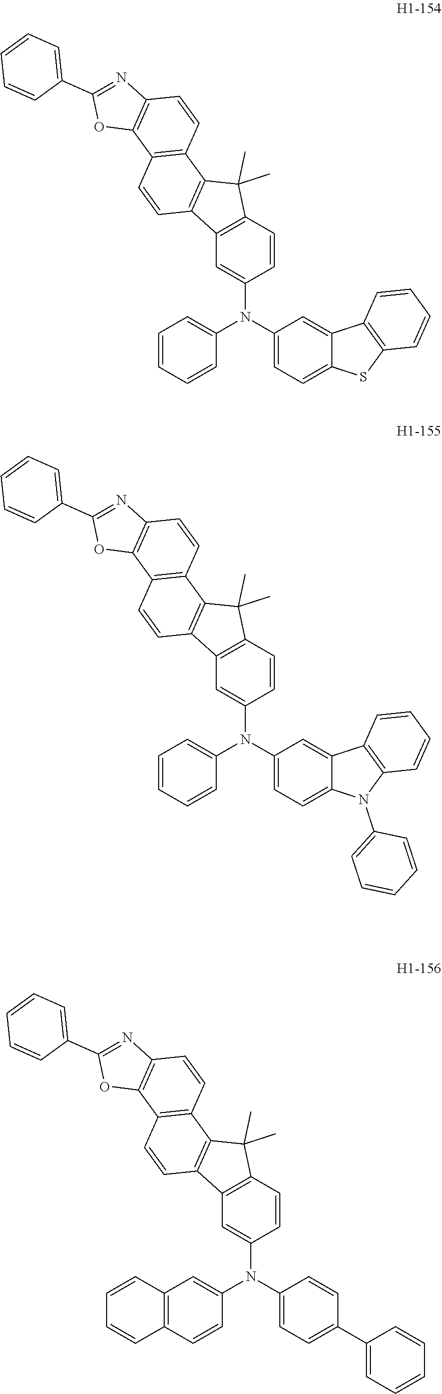 [SMILES: CC1(C)c2ccc(N(c3ccccc3)c3ccc4sc5ccccc5c4c3)cc2-c2ccc3c(ccc4nc(-c5ccccc5)oc43)c21 ; CC1(C)c2ccc(N(c3ccccc3)c3ccc4c(c3)c3ccccc3n4-c3ccccc3)cc2-c2ccc3c(ccc4nc(-c5ccccc5)oc43)c21 ; CC1(C)c2ccc(N(c3ccc(-c4ccccc4)cc3)c3ccc4ccccc4c3)cc2-c2ccc3c(ccc4nc(-c5ccccc5)oc43)c21]

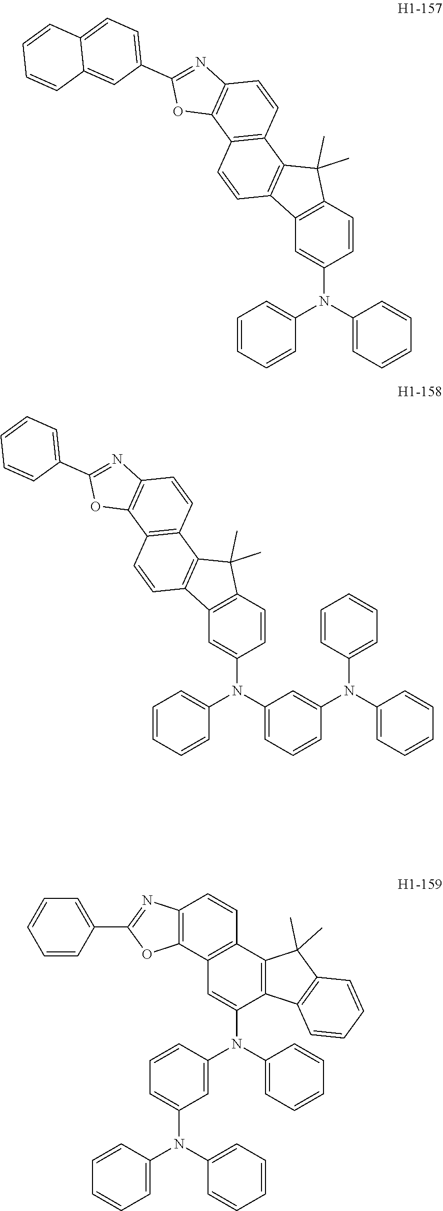 [SMILES: CC1(C)c2ccc(N(c3ccccc3)c3ccccc3)cc2-c2ccc3c(ccc4nc(-c5ccc6ccccc6c5)oc43)c21 ; CC1(C)c2ccc(N(c3ccccc3)c3cccc(N(c4ccccc4)c4ccccc4)c3)cc2-c2ccc3c(ccc4nc(-c5ccccc5)oc43)c21 ; CC1(C)c2ccccc2-c2c(N(c3ccccc3)c3cccc(N(c4ccccc4)c4ccccc4)c3)cc3c(ccc4nc(-c5ccccc5)oc43)c21]

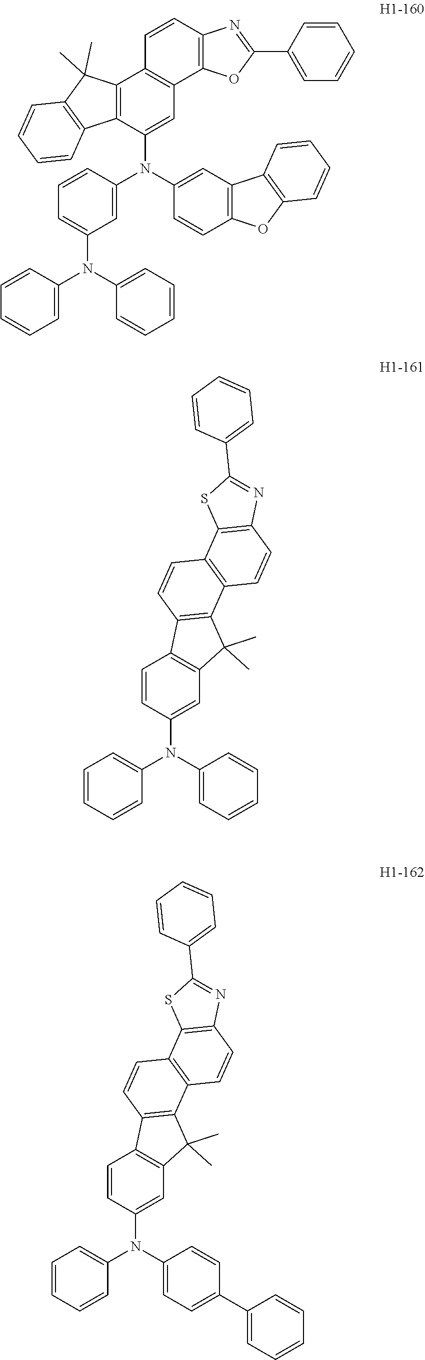 [SMILES: CC1(C)c2ccccc2-c2c(N(c3cccc(N(c4ccccc4)c4ccccc4)c3)c3ccc4oc5ccccc5c4c3)cc3c(ccc4nc(-c5ccccc5)oc43)c21 ; CC1(C)c2cc(N(c3ccccc3)c3ccccc3)ccc2-c2ccc3c(ccc4nc(-c5ccccc5)sc43)c21 ; CC1(C)c2cc(N(c3ccccc3)c3ccc(-c4ccccc4)cc3)ccc2-c2ccc3c(ccc4nc(-c5ccccc5)sc43)c21]

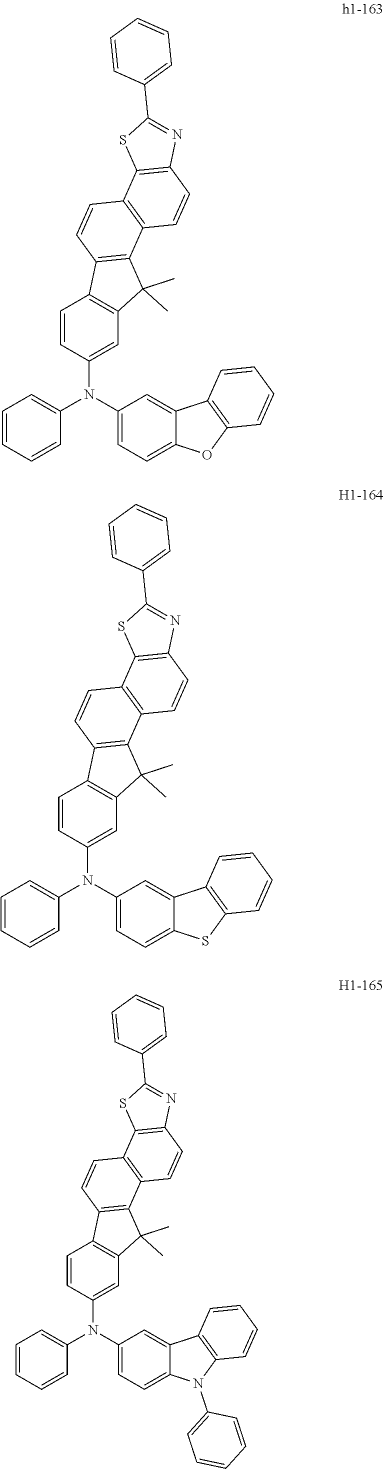

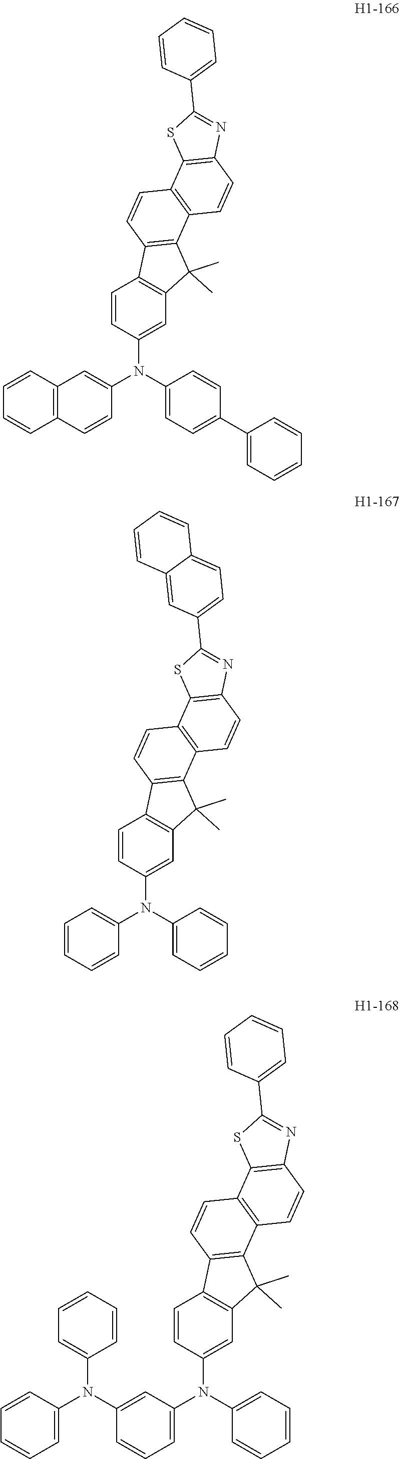 [SMILES: CC1(C)c2cc(N(c3ccc(-c4ccccc4)cc3)c3ccc4ccccc4c3)ccc2-c2ccc3c(ccc4nc(-c5ccccc5)sc43)c21 ; CC1(C)c2cc(N(c3ccccc3)c3ccccc3)ccc2-c2ccc3c(ccc4nc(-c5ccc6ccccc6c5)sc43)c21 ; CC1(C)c2cc(N(c3ccccc3)c3cccc(N(c4ccccc4)c4ccccc4)c3)ccc2-c2ccc3c(ccc4nc(-c5ccccc5)sc43)c21]

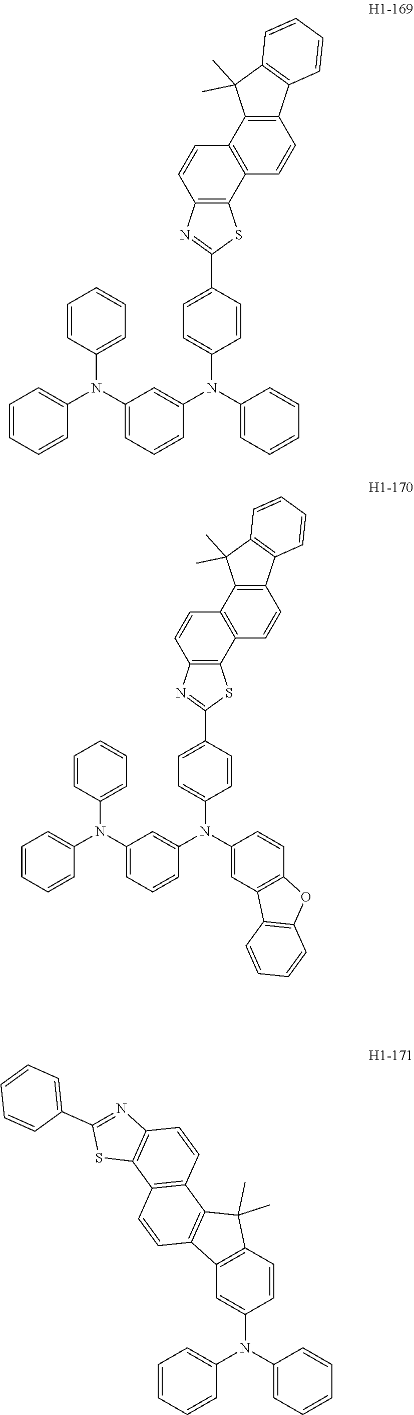

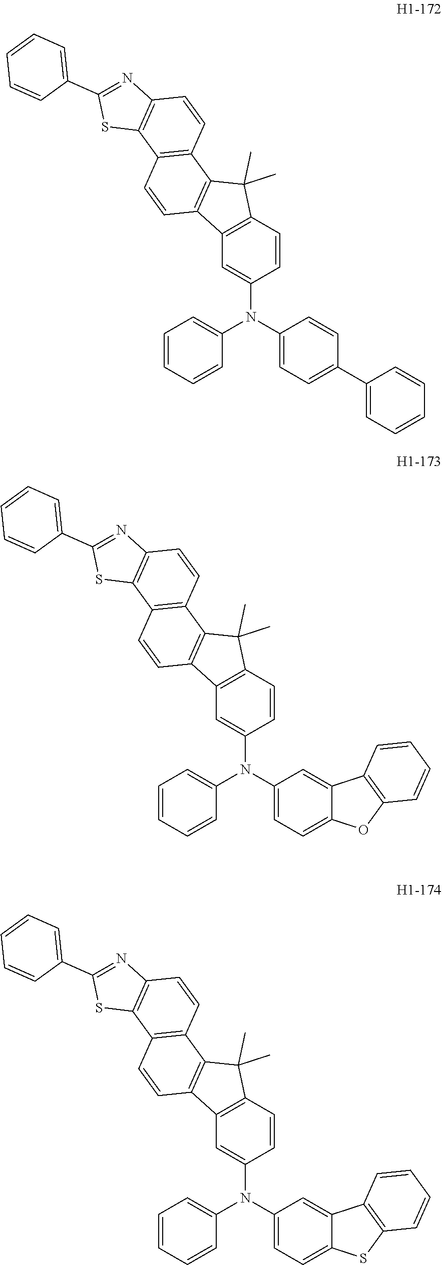 [SMILES: CC1(C)c2ccc(N(c3ccccc3)c3ccc(-c4ccccc4)cc3)cc2-c2ccc3c(ccc4nc(-c5ccccc5)sc43)c21 ; CC1(C)c2ccc(N(c3ccccc3)c3ccc4oc5ccccc5c4c3)cc2-c2ccc3c(ccc4nc(-c5ccccc5)sc43)c21 ; CC1(C)c2ccc(N(c3ccccc3)c3ccc4sc5ccccc5c4c3)cc2-c2ccc3c(ccc4nc(-c5ccccc5)sc43)c21]

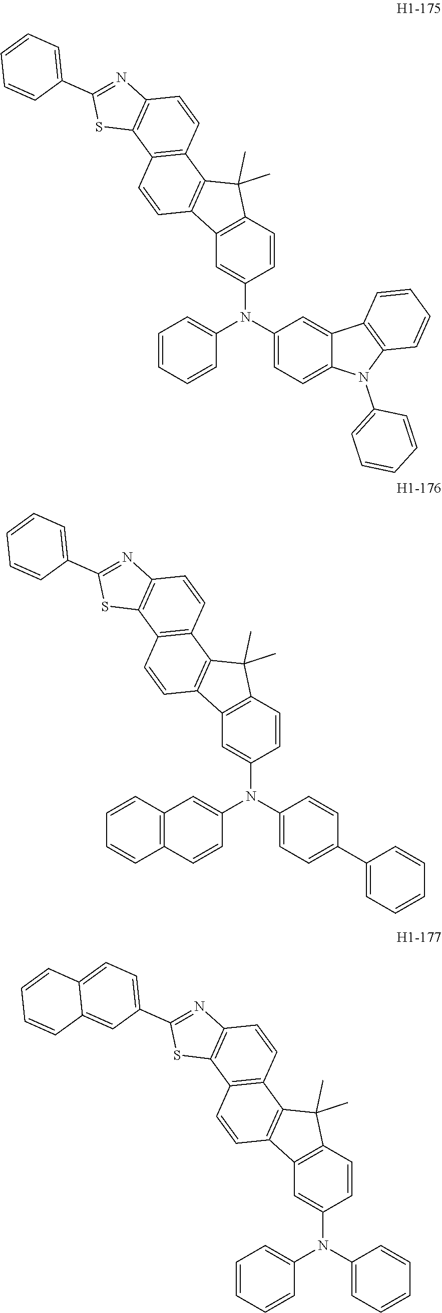 [SMILES: CC1(C)c2ccc(N(c3ccccc3)c3ccc4c(c3)c3ccccc3n4-c3ccccc3)cc2-c2ccc3c(ccc4nc(-c5ccccc5)sc43)c21 ; CC1(C)c2ccc(N(c3ccc(-c4ccccc4)cc3)c3ccc4ccccc4c3)cc2-c2ccc3c(ccc4nc(-c5ccccc5)sc43)c21 ; CC1(C)c2ccc(N(c3ccccc3)c3ccccc3)cc2-c2ccc3c(ccc4nc(-c5ccc6ccccc6c5)sc43)c21]

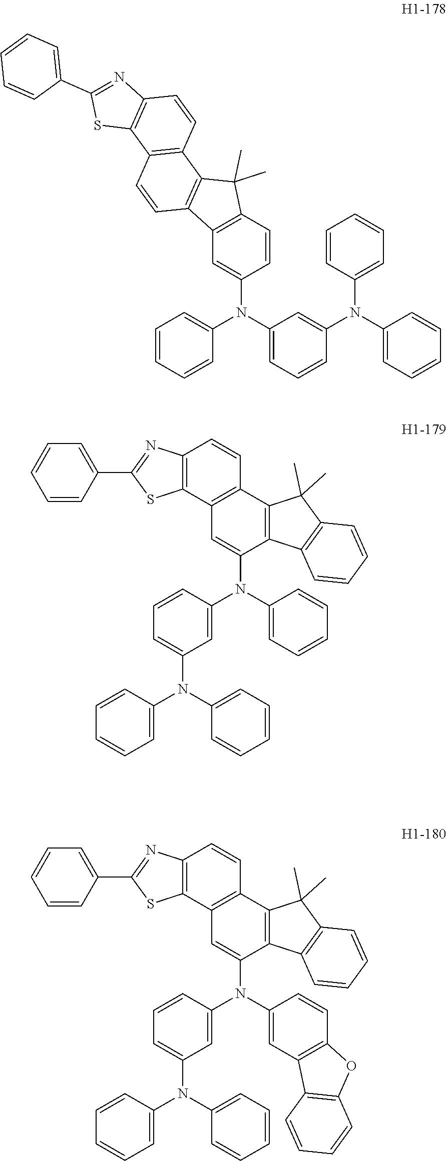 [SMILES: CC1(C)c2ccc(N(c3ccccc3)c3cccc(N(c4ccccc4)c4ccccc4)c3)cc2-c2ccc3c(ccc4nc(-c5ccccc5)sc43)c21 ; CC1(C)c2ccccc2-c2c(N(c3ccccc3)c3cccc(N(c4ccccc4)c4ccccc4)c3)cc3c(ccc4nc(-c5ccccc5)sc43)c21 ; CC1(C)c2ccccc2-c2c(N(c3cccc(N(c4ccccc4)c4ccccc4)c3)c3ccc4oc5ccccc5c4c3)cc3c(ccc4nc(-c5ccccc5)sc43)c21]

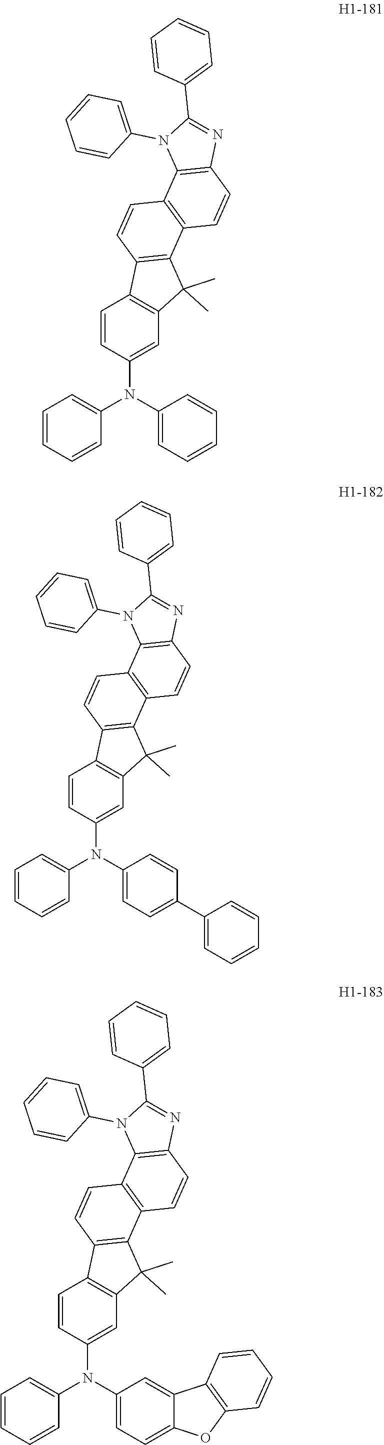 [SMILES: CC1(C)c2cc(N(c3ccccc3)c3ccccc3)ccc2-c2ccc3c(ccc4nc(-c5ccccc5)n(-c5ccccc5)c43)c21 ; CC1(C)c2cc(N(c3ccccc3)c3ccc(-c4ccccc4)cc3)ccc2-c2ccc3c(ccc4nc(-c5ccccc5)n(-c5ccccc5)c43)c21 ; CC1(C)c2cc(N(c3ccccc3)c3ccc4oc5ccccc5c4c3)ccc2-c2ccc3c(ccc4nc(-c5ccccc5)n(-c5ccccc5)c43)c21]

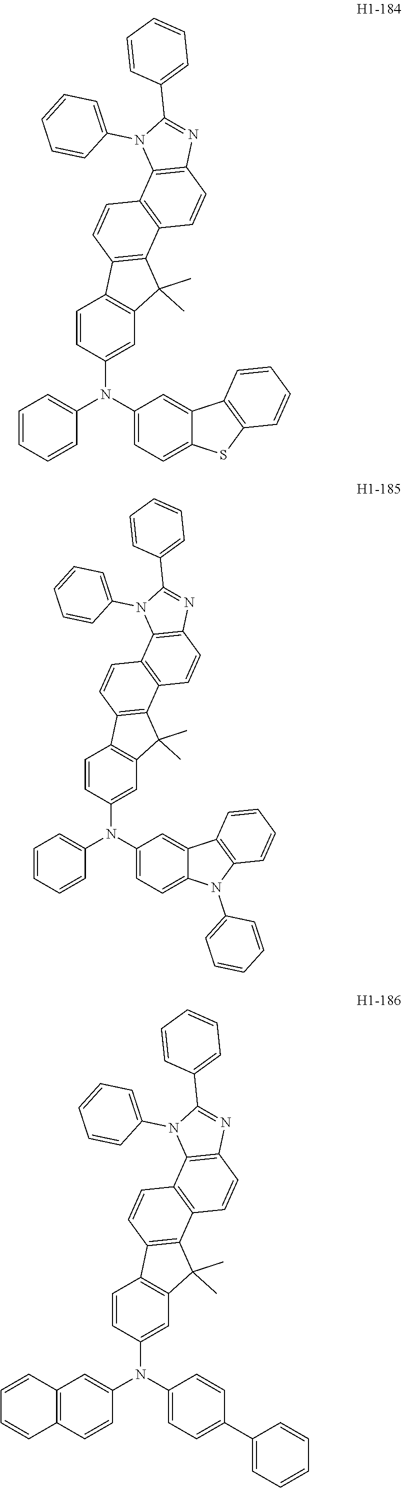

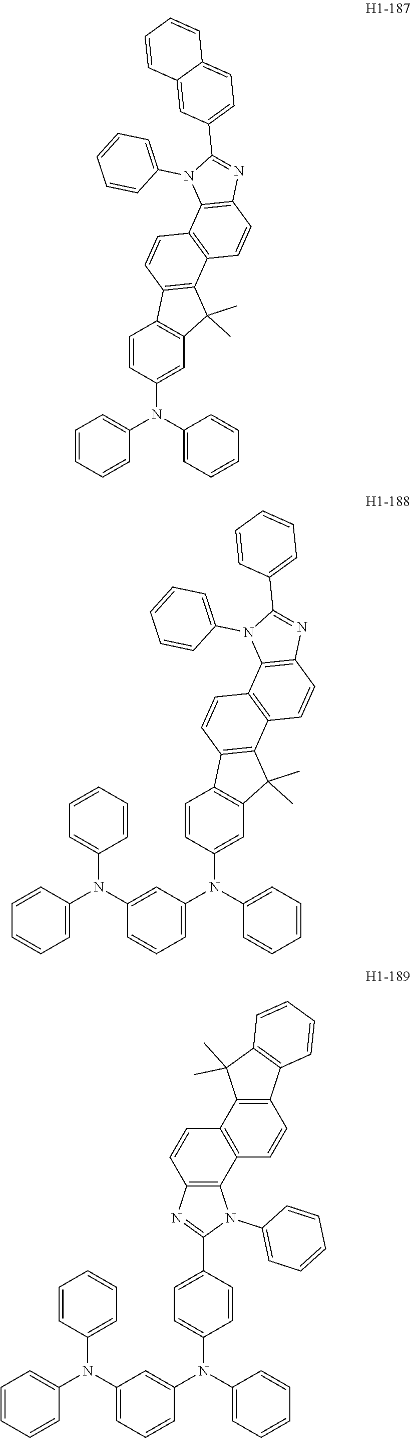 [SMILES: CC1(C)c2cc(N(c3ccccc3)c3ccccc3)ccc2-c2ccc3c(ccc4nc(-c5ccc6ccccc6c5)n(-c5ccccc5)c43)c21 ; CC1(C)c2cc(N(c3ccccc3)c3cccc(N(c4ccccc4)c4ccccc4)c3)ccc2-c2ccc3c(ccc4nc(-c5ccccc5)n(-c5ccccc5)c43)c21 ; CC1(C)c2ccccc2-c2ccc3c(ccc4nc(-c5ccc(N(c6ccccc6)c6cccc(N(c7ccccc7)c7ccccc7)c6)cc5)n(-c5ccccc5)c43)c21]

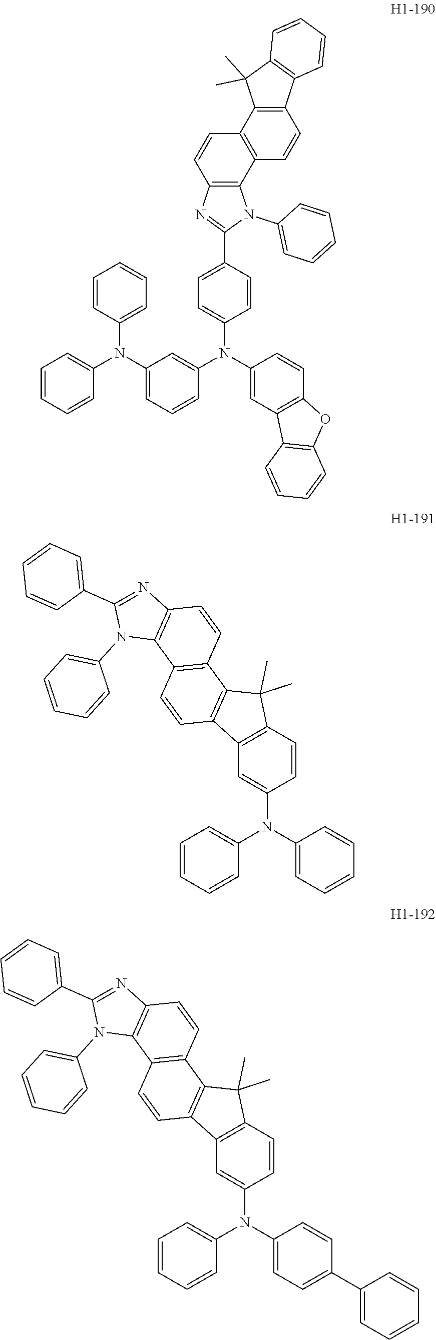 [SMILES: CC1(C)c2ccccc2-c2ccc3c(ccc4nc(-c5ccc(N(c6cccc(N(c7ccccc7)c7ccccc7)c6)c6ccc7oc8ccccc8c7c6)cc5)n(-c5ccccc5)c43)c21 ; CC1(C)c2ccc(N(c3ccccc3)c3ccccc3)cc2-c2ccc3c(ccc4nc(-c5ccccc5)n(-c5ccccc5)c43)c21 ; CC1(C)c2ccc(N(c3ccccc3)c3ccc(-c4ccccc4)cc3)cc2-c2ccc3c(ccc4nc(-c5ccccc5)n(-c5ccccc5)c43)c21]

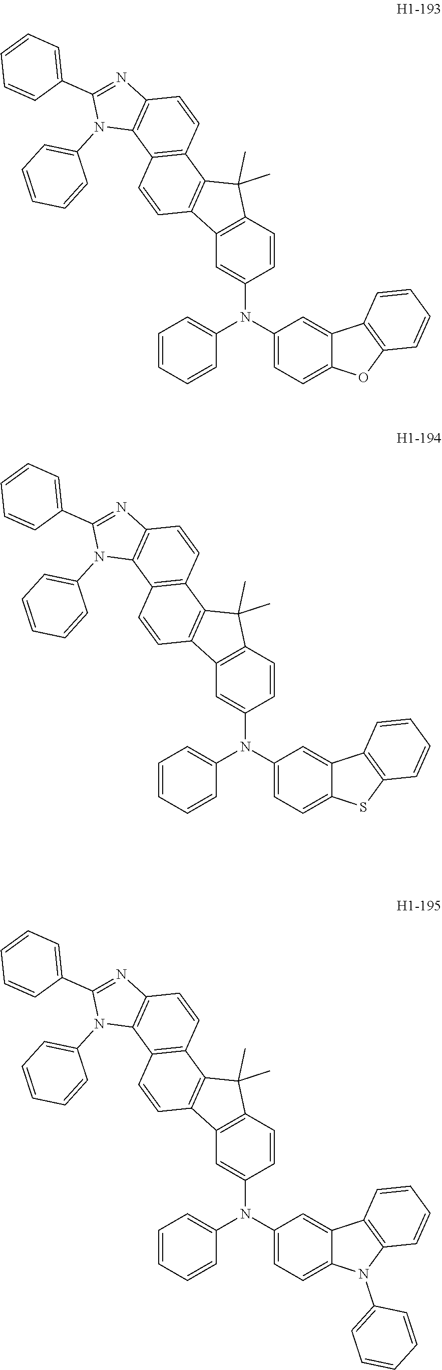 [SMILES: CC1(C)c2ccc(N(c3ccccc3)c3ccc4oc5ccccc5c4c3)cc2-c2ccc3c(ccc4nc(-c5ccccc5)n(-c5ccccc5)c43)c21 ; CC1(C)c2ccc(N(c3ccccc3)c3ccc4sc5ccccc5c4c3)cc2-c2ccc3c(ccc4nc(-c5ccccc5)n(-c5ccccc5)c43)c21 ; CC1(C)c2ccc(N(c3ccccc3)c3ccc4c(c3)c3ccccc3n4-c3ccccc3)cc2-c2ccc3c(ccc4nc(-c5ccccc5)n(-c5ccccc5)c43)c21]

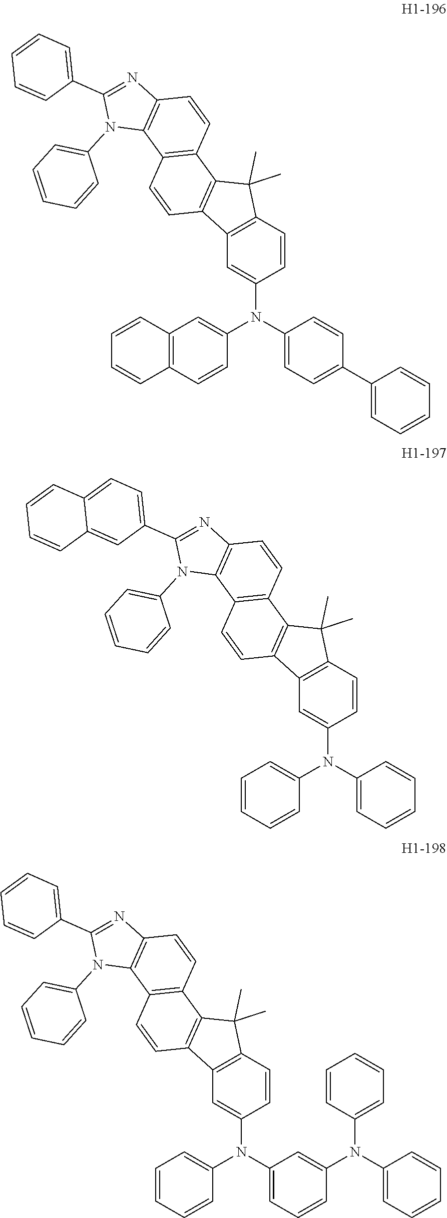 [SMILES: CC1(C)c2ccc(N(c3ccc(-c4ccccc4)cc3)c3ccc4ccccc4c3)cc2-c2ccc3c(ccc4nc(-c5ccccc5)n(-c5ccccc5)c43)c21 ; CC1(C)c2ccc(N(c3ccccc3)c3ccccc3)cc2-c2ccc3c(ccc4nc(-c5ccc6ccccc6c5)n(-c5ccccc5)c43)c21 ; CC1(C)c2ccc(N(c3ccccc3)c3cccc(N(c4ccccc4)c4ccccc4)c3)cc2-c2ccc3c(ccc4nc(-c5ccccc5)n(-c5ccccc5)c43)c21]

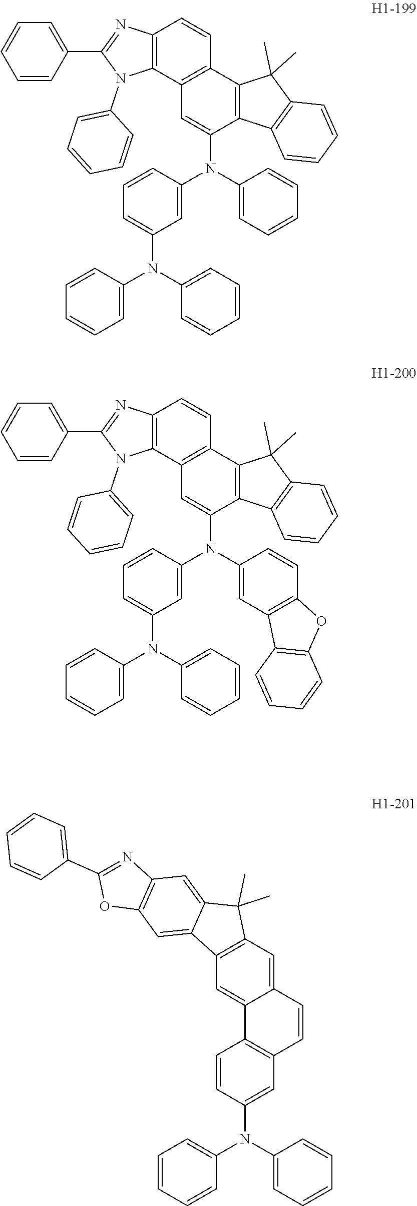 [SMILES: CC1(C)c2ccccc2-c2c(N(c3ccccc3)c3cccc(N(c4ccccc4)c4ccccc4)c3)cc3c(ccc4nc(-c5ccccc5)n(-c5ccccc5)c43)c21 ; CC1(C)c2ccccc2-c2c(N(c3cccc(N(c4ccccc4)c4ccccc4)c3)c3ccc4oc5ccccc5c4c3)cc3c(ccc4nc(-c5ccccc5)n(-c5ccccc5)c43)c21 ; CC1(C)c2cc3ccc4cc(N(c5ccccc5)c5ccccc5)ccc4c3cc2-c2cc3oc(-c4ccccc4)nc3cc21]

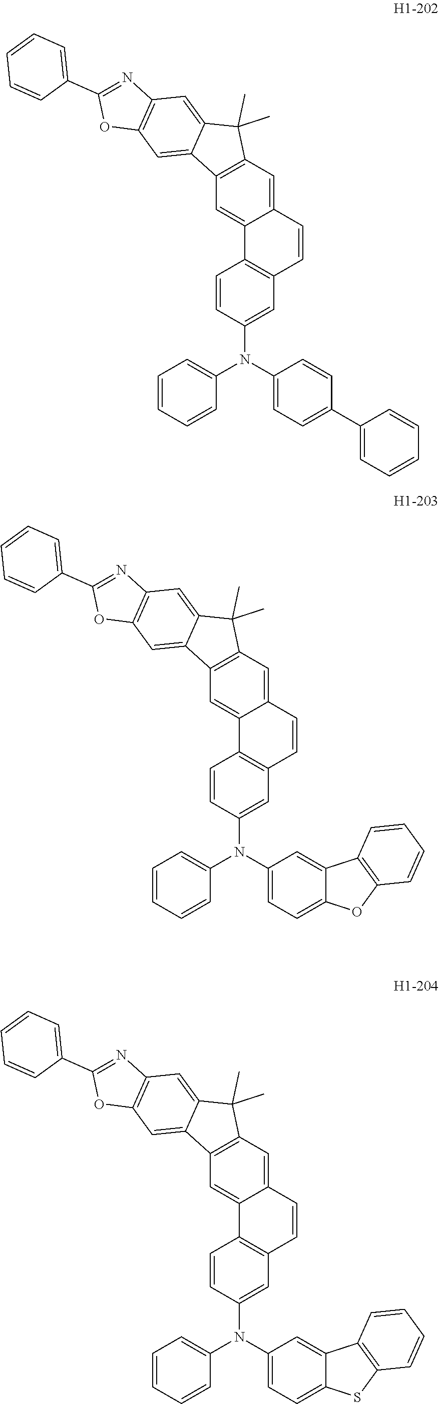 [SMILES: CC1(C)c2cc3ccc4cc(N(c5ccccc5)c5ccc(-c6ccccc6)cc5)ccc4c3cc2-c2cc3oc(-c4ccccc4)nc3cc21 ; CC1(C)c2cc3ccc4cc(N(c5ccccc5)c5ccc6oc7ccccc7c6c5)ccc4c3cc2-c2cc3oc(-c4ccccc4)nc3cc21 ; CC1(C)c2cc3ccc4cc(N(c5ccccc5)c5ccc6sc7ccccc7c6c5)ccc4c3cc2-c2cc3oc(-c4ccccc4)nc3cc21]

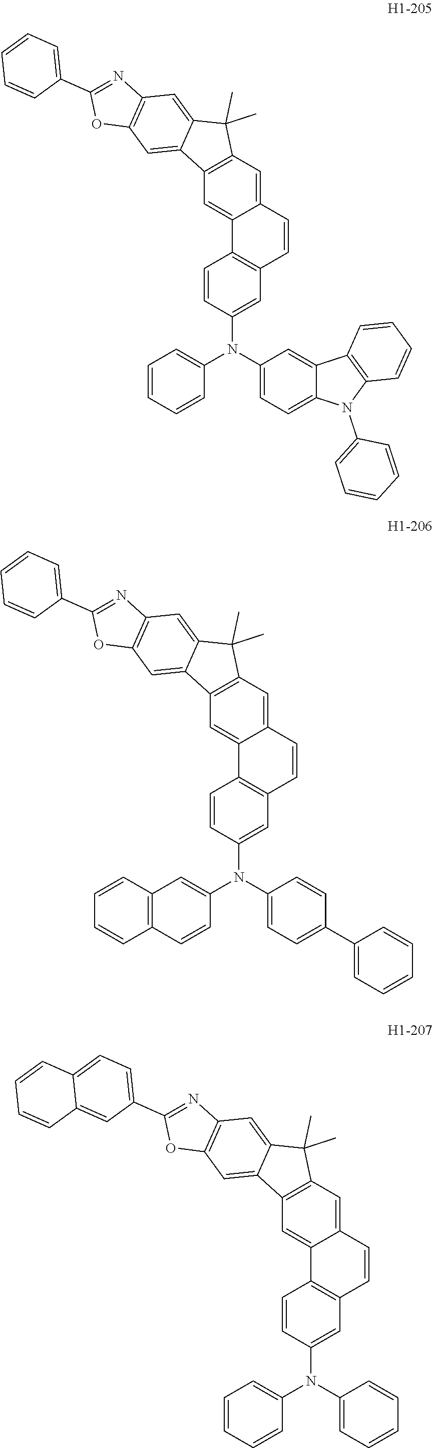 [SMILES: CC1(C)c2cc3ccc4cc(N(c5ccccc5)c5ccc6c(c5)c5ccccc5n6-c5ccccc5)ccc4c3cc2-c2cc3oc(-c4ccccc4)nc3cc21 ; CC1(C)c2cc3ccc4cc(N(c5ccc(-c6ccccc6)cc5)c5ccc6ccccc6c5)ccc4c3cc2-c2cc3oc(-c4ccccc4)nc3cc21 ; CC1(C)c2cc3ccc4cc(N(c5ccccc5)c5ccccc5)ccc4c3cc2-c2cc3oc(-c4ccc5ccccc5c4)nc3cc21]

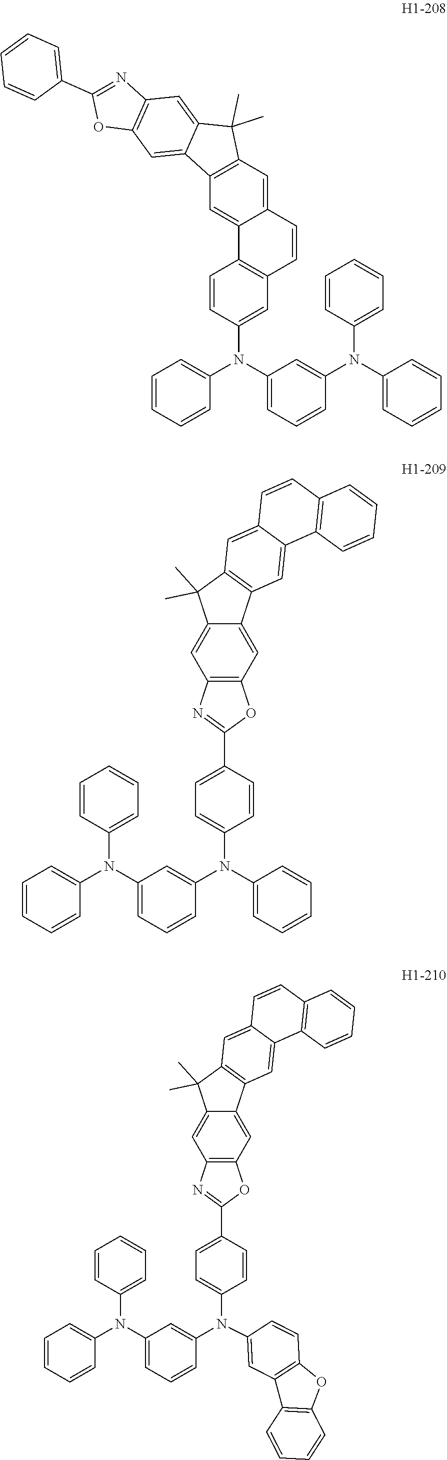 [SMILES: CC1(C)c2cc3ccc4cc(N(c5ccccc5)c5cccc(N(c6ccccc6)c6ccccc6)c5)ccc4c3cc2-c2cc3oc(-c4ccccc4)nc3cc21 ; CC1(C)c2cc3ccc4ccccc4c3cc2-c2cc3oc(-c4ccc(N(c5ccccc5)c5cccc(N(c6ccccc6)c6ccccc6)c5)cc4)nc3cc21 ; CC1(C)c2cc3ccc4ccccc4c3cc2-c2cc3oc(-c4ccc(N(c5cccc(N(c6ccccc6)c6ccccc6)c5)c5ccc6oc7ccccc7c6c5)cc4)nc3cc21]

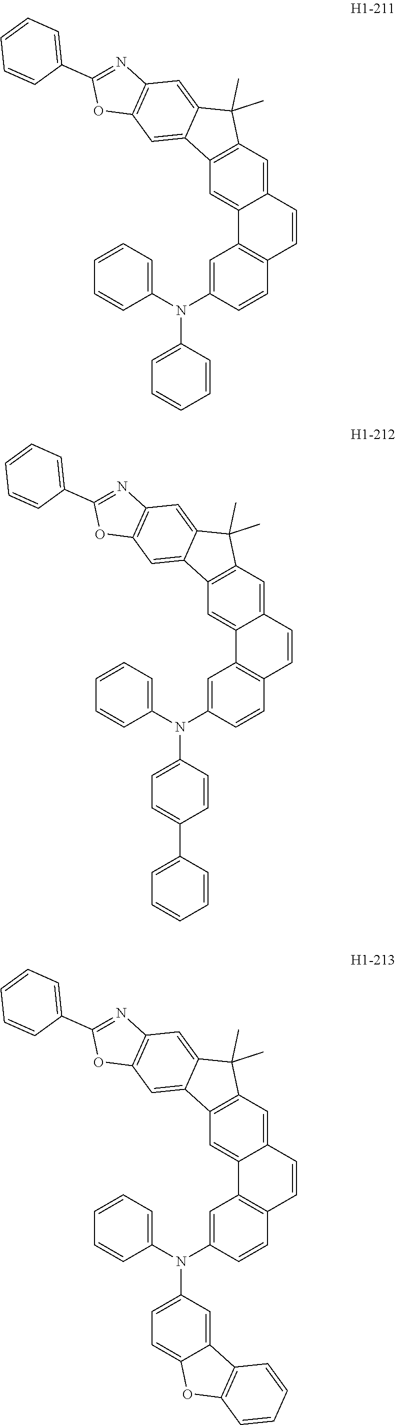 [SMILES: CC1(C)c2cc3ccc4ccc(N(c5ccccc5)c5ccccc5)cc4c3cc2-c2cc3oc(-c4ccccc4)nc3cc21 ; CC1(C)c2cc3ccc4ccc(N(c5ccccc5)c5ccc(-c6ccccc6)cc5)cc4c3cc2-c2cc3oc(-c4ccccc4)nc3cc21 ; CC1(C)c2cc3ccc4ccc(N(c5ccccc5)c5ccc6oc7ccccc7c6c5)cc4c3cc2-c2cc3oc(-c4ccccc4)nc3cc21]

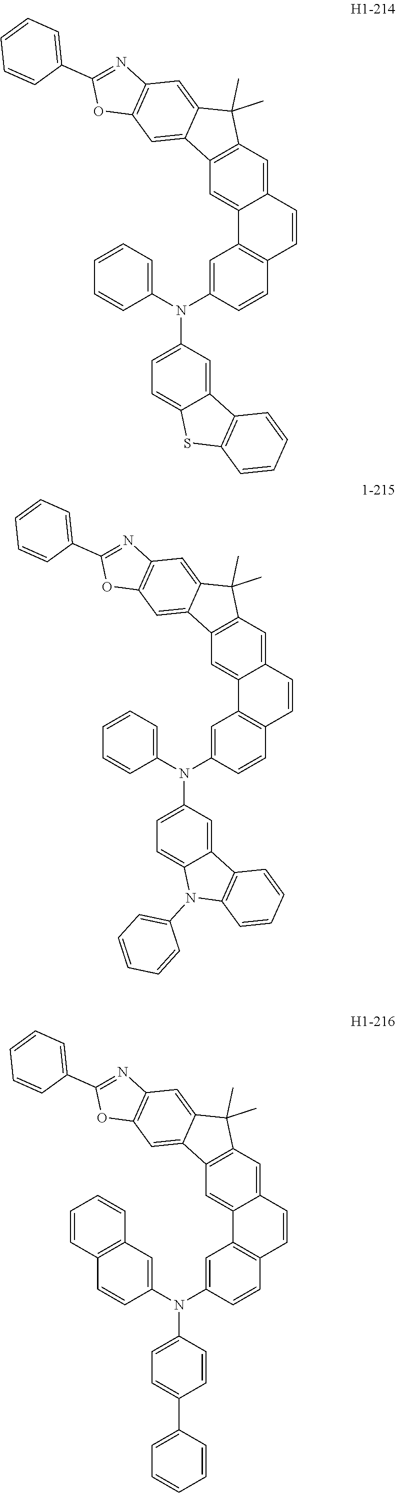 [SMILES: CC1(C)c2cc3ccc4ccc(N(c5ccccc5)c5ccc6sc7ccccc7c6c5)cc4c3cc2-c2cc3oc(-c4ccccc4)nc3cc21 ; CC1(C)c2cc3ccc4ccc(N(c5ccccc5)c5ccc6c(c5)c5ccccc5n6-c5ccccc5)cc4c3cc2-c2cc3oc(-c4ccccc4)nc3cc21 ; CC1(C)c2cc3ccc4ccc(N(c5ccc(-c6ccccc6)cc5)c5ccc6ccccc6c5)cc4c3cc2-c2cc3oc(-c4ccccc4)nc3cc21]

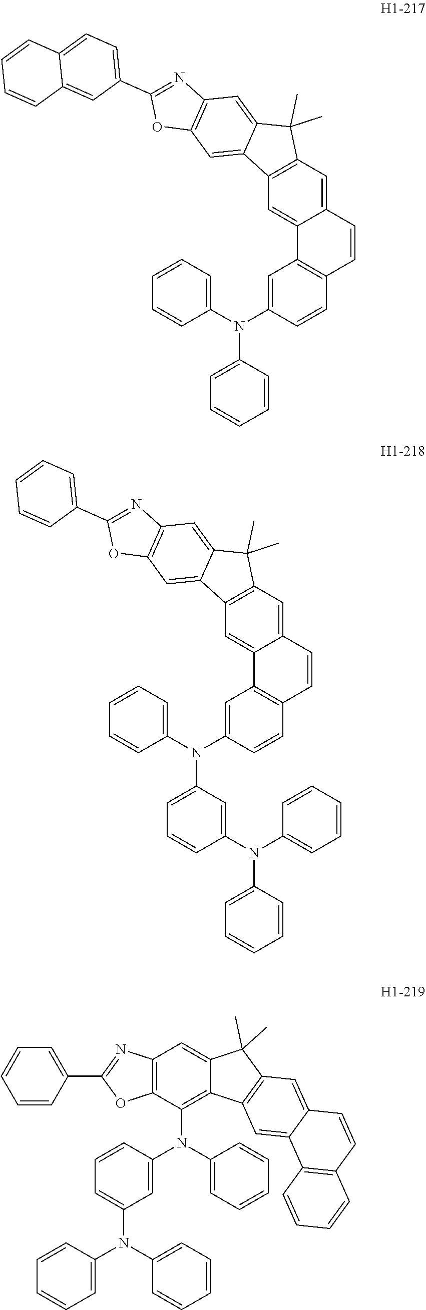 [SMILES: CC1(C)c2cc3ccc4ccc(N(c5ccccc5)c5ccccc5)cc4c3cc2-c2cc3oc(-c4ccc5ccccc5c4)nc3cc21 ; CC1(C)c2cc3ccc4ccc(N(c5ccccc5)c5cccc(N(c6ccccc6)c6ccccc6)c5)cc4c3cc2-c2cc3oc(-c4ccccc4)nc3cc21 ; CC1(C)c2cc3ccc4ccccc4c3cc2-c2c1cc1nc(-c3ccccc3)oc1c2N(c1ccccc1)c1cccc(N(c2ccccc2)c2ccccc2)c1]

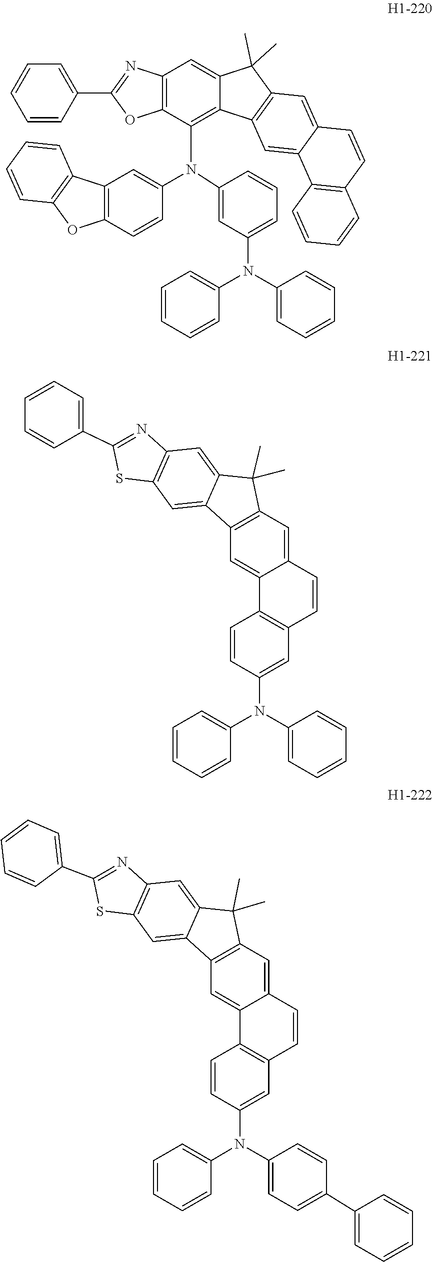 [SMILES: CC1(C)c2cc3ccc4ccccc4c3cc2-c2c1cc1nc(-c3ccccc3)oc1c2N(c1cccc(N(c2ccccc2)c2ccccc2)c1)c1ccc2oc3ccccc3c2c1 ; CC1(C)c2cc3ccc4cc(N(c5ccccc5)c5ccccc5)ccc4c3cc2-c2cc3sc(-c4ccccc4)nc3cc21 ; CC1(C)c2cc3ccc4cc(N(c5ccccc5)c5ccc(-c6ccccc6)cc5)ccc4c3cc2-c2cc3sc(-c4ccccc4)nc3cc21]

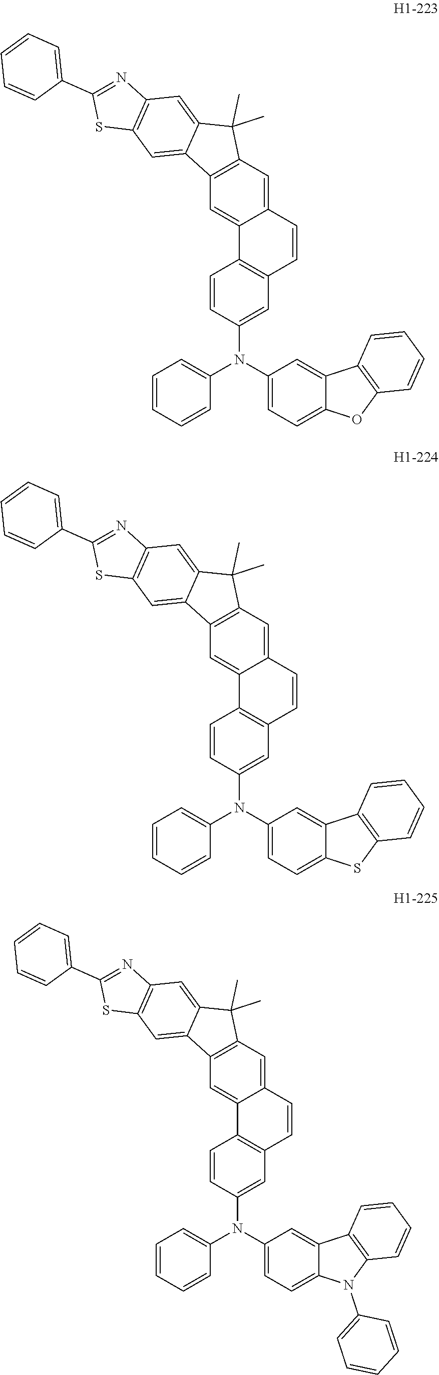 [SMILES: CC1(C)c2cc3ccc4cc(N(c5ccccc5)c5ccc6oc7ccccc7c6c5)ccc4c3cc2-c2cc3sc(-c4ccccc4)nc3cc21 ; CC1(C)c2cc3ccc4cc(N(c5ccccc5)c5ccc6sc7ccccc7c6c5)ccc4c3cc2-c2cc3sc(-c4ccccc4)nc3cc21 ; CC1(C)c2cc3ccc4cc(N(c5ccccc5)c5ccc6c(c5)c5ccccc5n6-c5ccccc5)ccc4c3cc2-c2cc3sc(-c4ccccc4)nc3cc21]

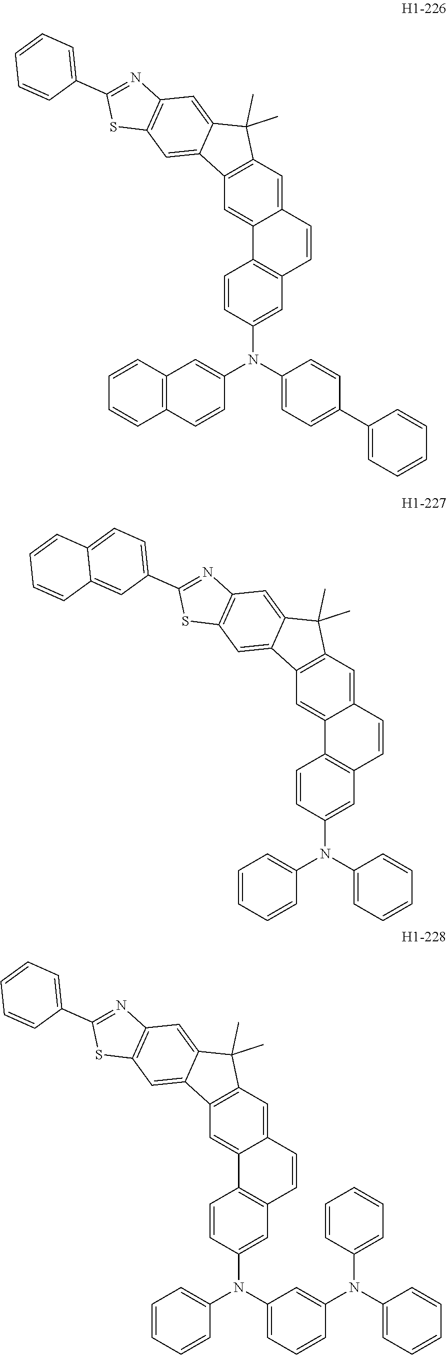 [SMILES: CC1(C)c2cc3ccc4cc(N(c5ccc(-c6ccccc6)cc5)c5ccc6ccccc6c5)ccc4c3cc2-c2cc3sc(-c4ccccc4)nc3cc21 ; CC1(C)c2cc3ccc4cc(N(c5ccccc5)c5ccccc5)ccc4c3cc2-c2cc3sc(-c4ccc5ccccc5c4)nc3cc21 ; CC1(C)c2cc3ccc4cc(N(c5ccccc5)c5cccc(N(c6ccccc6)c6ccccc6)c5)ccc4c3cc2-c2cc3sc(-c4ccccc4)nc3cc21]

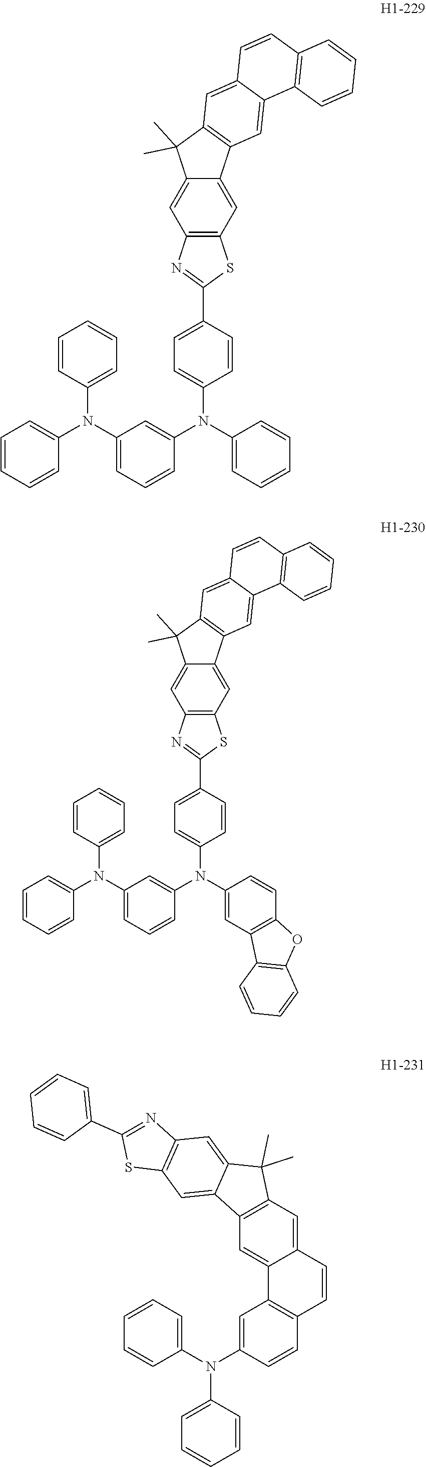 [SMILES: CC1(C)c2cc3ccc4ccccc4c3cc2-c2cc3sc(-c4ccc(N(c5ccccc5)c5cccc(N(c6ccccc6)c6ccccc6)c5)cc4)nc3cc21 ; CC1(C)c2cc3ccc4ccccc4c3cc2-c2cc3sc(-c4ccc(N(c5cccc(N(c6ccccc6)c6ccccc6)c5)c5ccc6oc7ccccc7c6c5)cc4)nc3cc21 ; CC1(C)c2cc3ccc4ccc(N(c5ccccc5)c5ccccc5)cc4c3cc2-c2cc3sc(-c4ccccc4)nc3cc21]

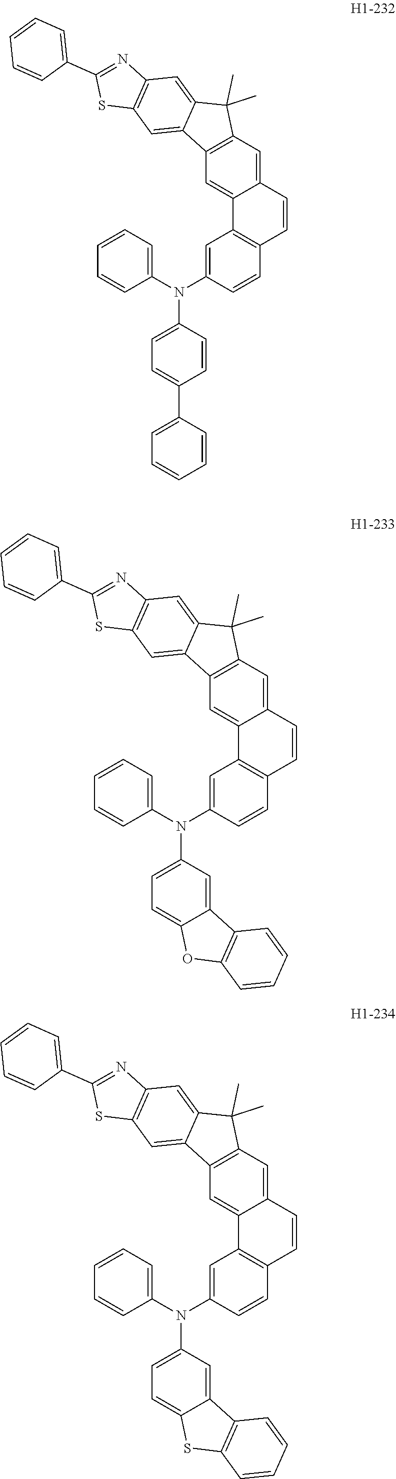 [SMILES: CC1(C)c2cc3ccc4ccc(N(c5ccccc5)c5ccc(-c6ccccc6)cc5)cc4c3cc2-c2cc3sc(-c4ccccc4)nc3cc21 ; CC1(C)c2cc3ccc4ccc(N(c5ccccc5)c5ccc6oc7ccccc7c6c5)cc4c3cc2-c2cc3sc(-c4ccccc4)nc3cc21 ; CC1(C)c2cc3ccc4ccc(N(c5ccccc5)c5ccc6sc7ccccc7c6c5)cc4c3cc2-c2cc3sc(-c4ccccc4)nc3cc21]

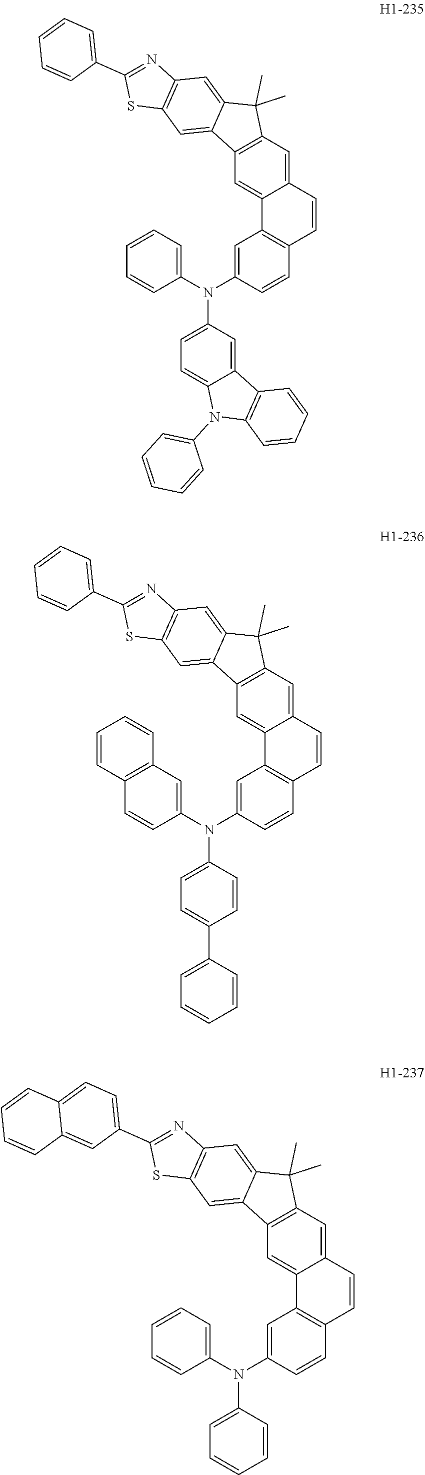 [SMILES: CC1(C)c2cc3ccc4ccc(N(c5ccccc5)c5ccc6c(c5)c5ccccc5n6-c5ccccc5)cc4c3cc2-c2cc3sc(-c4ccccc4)nc3cc21 ; CC1(C)c2cc3ccc4ccc(N(c5ccc(-c6ccccc6)cc5)c5ccc6ccccc6c5)cc4c3cc2-c2cc3sc(-c4ccccc4)nc3cc21 ; CC1(C)c2cc3ccc4ccc(N(c5ccccc5)c5ccccc5)cc4c3cc2-c2cc3sc(-c4ccc5ccccc5c4)nc3cc21]

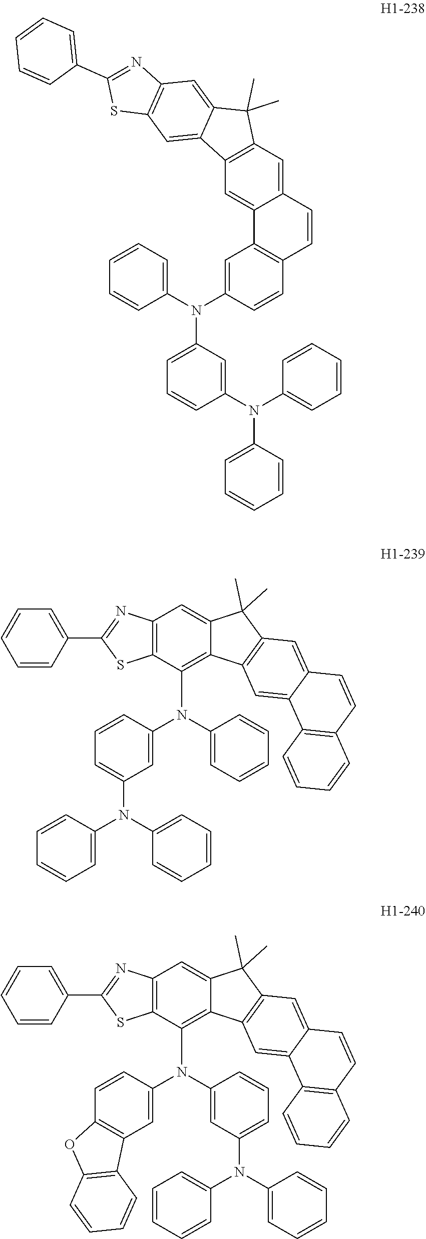 [SMILES: CC1(C)c2cc3ccc4ccc(N(c5ccccc5)c5cccc(N(c6ccccc6)c6ccccc6)c5)cc4c3cc2-c2cc3sc(-c4ccccc4)nc3cc21 ; CC1(C)c2cc3ccc4ccccc4c3cc2-c2c1cc1nc(-c3ccccc3)sc1c2N(c1ccccc1)c1cccc(N(c2ccccc2)c2ccccc2)c1 ; CC1(C)c2cc3ccc4ccccc4c3cc2-c2c1cc1nc(-c3ccccc3)sc1c2N(c1cccc(N(c2ccccc2)c2ccccc2)c1)c1ccc2oc3ccccc3c2c1]

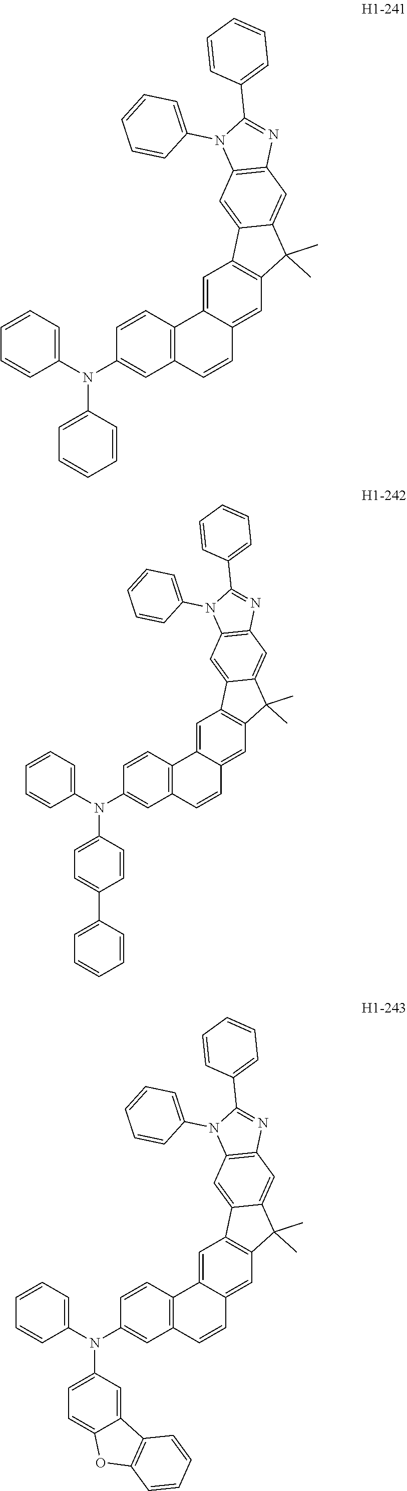 [SMILES: CC1(C)c2cc3ccc4cc(N(c5ccccc5)c5ccccc5)ccc4c3cc2-c2cc3c(cc21)nc(-c1ccccc1)n3-c1ccccc1 ; CC1(C)c2cc3ccc4cc(N(c5ccccc5)c5ccc(-c6ccccc6)cc5)ccc4c3cc2-c2cc3c(cc21)nc(-c1ccccc1)n3-c1ccccc1 ; CC1(C)c2cc3ccc4cc(N(c5ccccc5)c5ccc6oc7ccccc7c6c5)ccc4c3cc2-c2cc3c(cc21)nc(-c1ccccc1)n3-c1ccccc1]

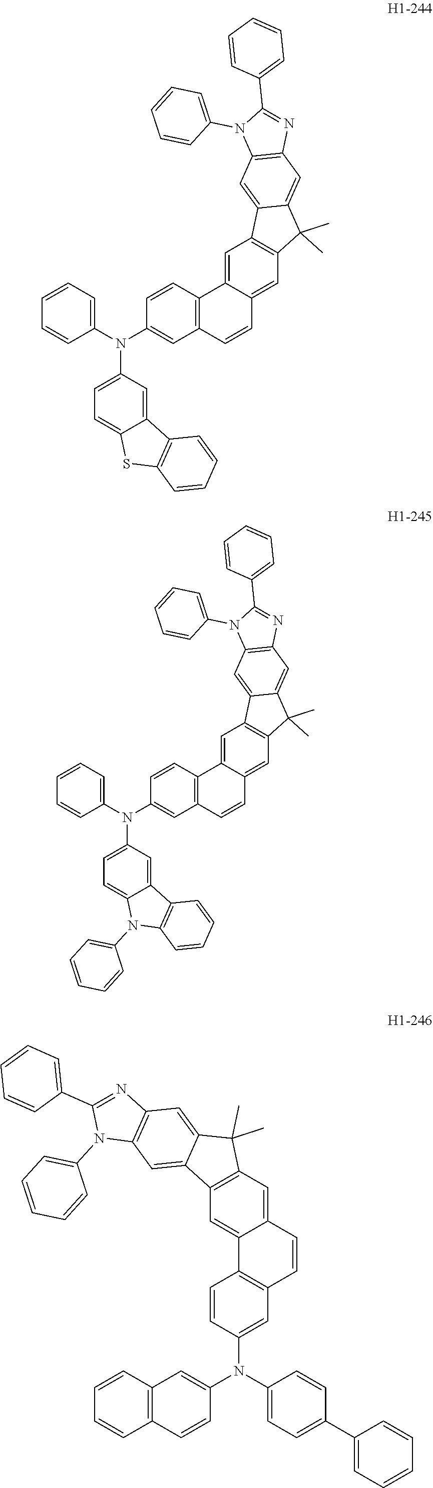 [SMILES: CC1(C)c2cc3ccc4cc(N(c5ccccc5)c5ccc6sc7ccccc7c6c5)ccc4c3cc2-c2cc3c(cc21)nc(-c1ccccc1)n3-c1ccccc1 ; CC1(C)c2cc3ccc4cc(N(c5ccccc5)c5ccc6c(c5)c5ccccc5n6-c5ccccc5)ccc4c3cc2-c2cc3c(cc21)nc(-c1ccccc1)n3-c1ccccc1 ; CC1(C)c2cc3ccc4cc(N(c5ccc(-c6ccccc6)cc5)c5ccc6ccccc6c5)ccc4c3cc2-c2cc3c(cc21)nc(-c1ccccc1)n3-c1ccccc1]

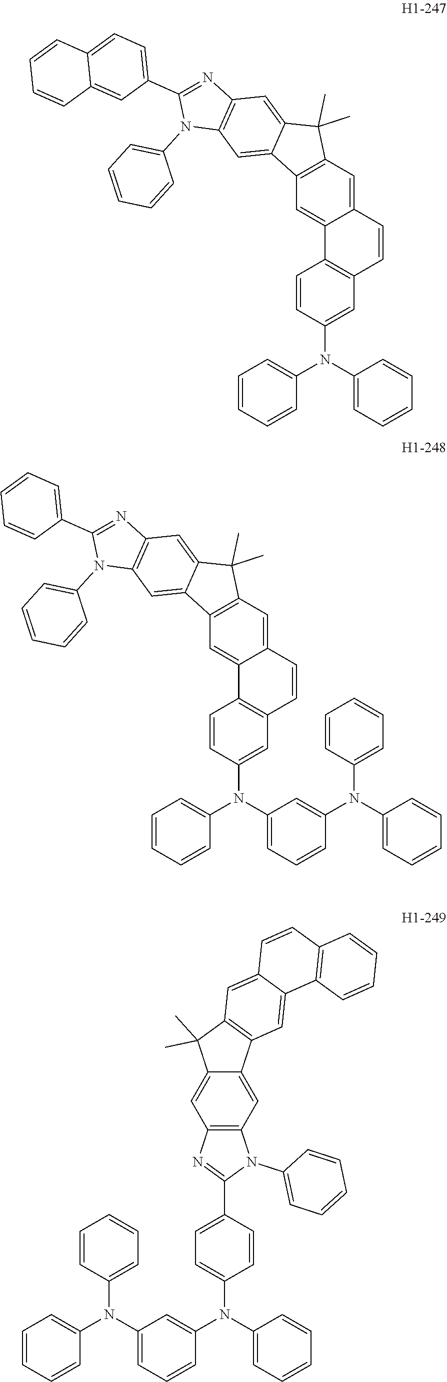 [SMILES: CC1(C)c2cc3ccc4cc(N(c5ccccc5)c5ccccc5)ccc4c3cc2-c2cc3c(cc21)nc(-c1ccc2ccccc2c1)n3-c1ccccc1 ; CC1(C)c2cc3ccc4cc(N(c5ccccc5)c5cccc(N(c6ccccc6)c6ccccc6)c5)ccc4c3cc2-c2cc3c(cc21)nc(-c1ccccc1)n3-c1ccccc1 ; CC1(C)c2cc3ccc4ccccc4c3cc2-c2cc3c(cc21)nc(-c1ccc(N(c2ccccc2)c2cccc(N(c4ccccc4)c4ccccc4)c2)cc1)n3-c1ccccc1]

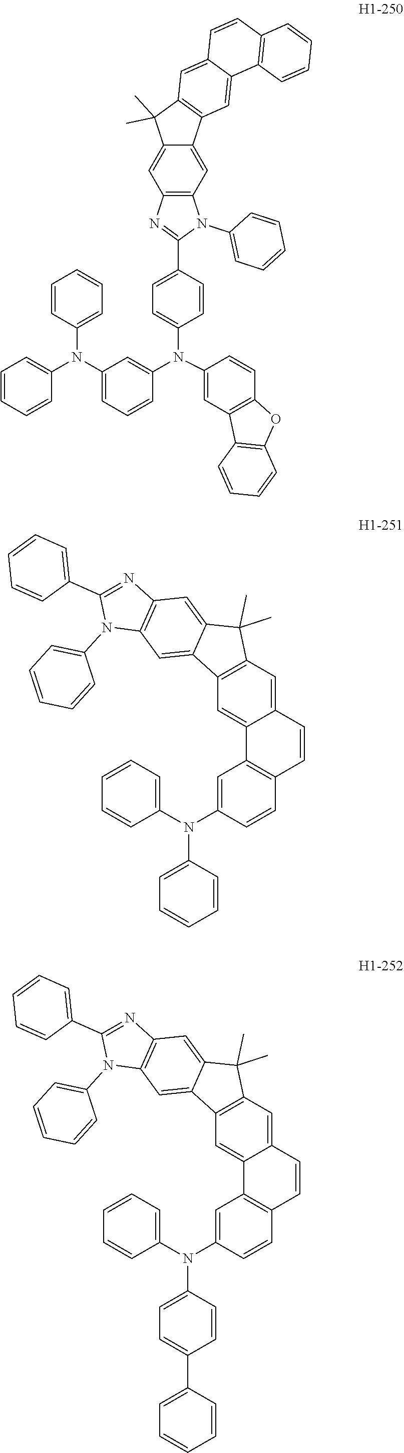 [SMILES: CC1(C)c2cc3ccc4ccccc4c3cc2-c2cc3c(cc21)nc(-c1ccc(N(c2cccc(N(c4ccccc4)c4ccccc4)c2)c2ccc4oc5ccccc5c4c2)cc1)n3-c1ccccc1 ; CC1(C)c2cc3ccc4ccc(N(c5ccccc5)c5ccccc5)cc4c3cc2-c2cc3c(cc21)nc(-c1ccccc1)n3-c1ccccc1 ; CC1(C)c2cc3ccc4ccc(N(c5ccccc5)c5ccc(-c6ccccc6)cc5)cc4c3cc2-c2cc3c(cc21)nc(-c1ccccc1)n3-c1ccccc1]

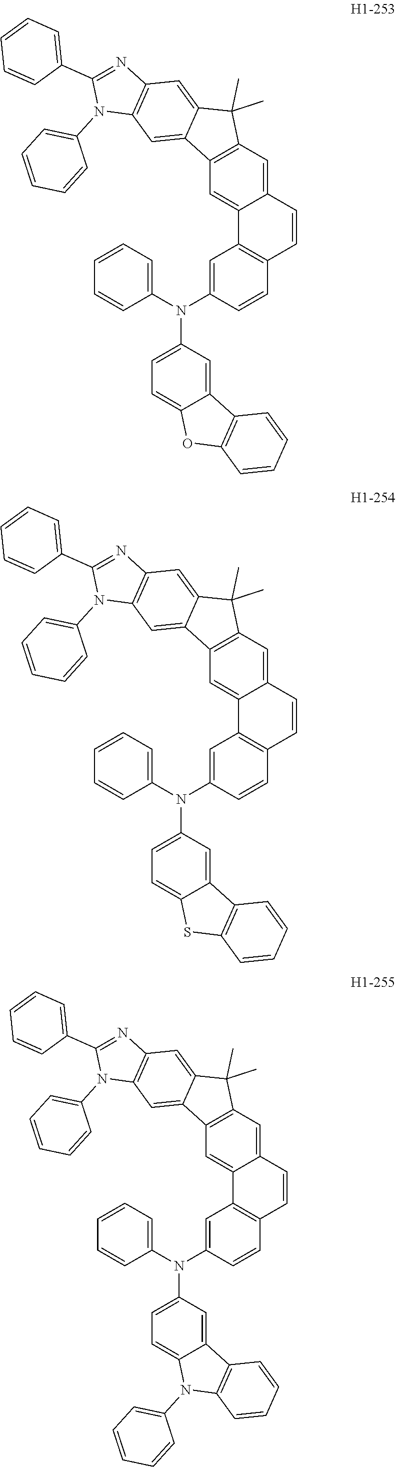 [SMILES: CC1(C)c2cc3ccc4ccc(N(c5ccccc5)c5ccc6oc7ccccc7c6c5)cc4c3cc2-c2cc3c(cc21)nc(-c1ccccc1)n3-c1ccccc1 ; CC1(C)c2cc3ccc4ccc(N(c5ccccc5)c5ccc6sc7ccccc7c6c5)cc4c3cc2-c2cc3c(cc21)nc(-c1ccccc1)n3-c1ccccc1 ; CC1(C)c2cc3ccc4ccc(N(c5ccccc5)c5ccc6c(c5)c5ccccc5n6-c5ccccc5)cc4c3cc2-c2cc3c(cc21)nc(-c1ccccc1)n3-c1ccccc1]

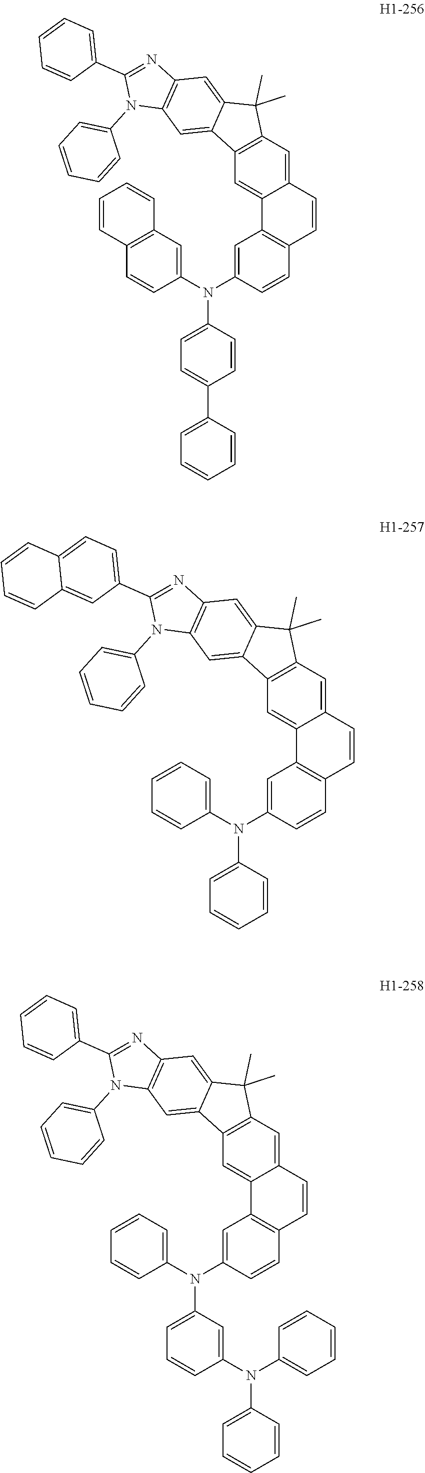 [SMILES: CC1(C)c2cc3ccc4ccc(N(c5ccc(-c6ccccc6)cc5)c5ccc6ccccc6c5)cc4c3cc2-c2cc3c(cc21)nc(-c1ccccc1)n3-c1ccccc1 ; CC1(C)c2cc3ccc4ccc(N(c5ccccc5)c5ccccc5)cc4c3cc2-c2cc3c(cc21)nc(-c1ccc2ccccc2c1)n3-c1ccccc1 ; CC1(C)c2cc3ccc4ccc(N(c5ccccc5)c5cccc(N(c6ccccc6)c6ccccc6)c5)cc4c3cc2-c2cc3c(cc21)nc(-c1ccccc1)n3-c1ccccc1]

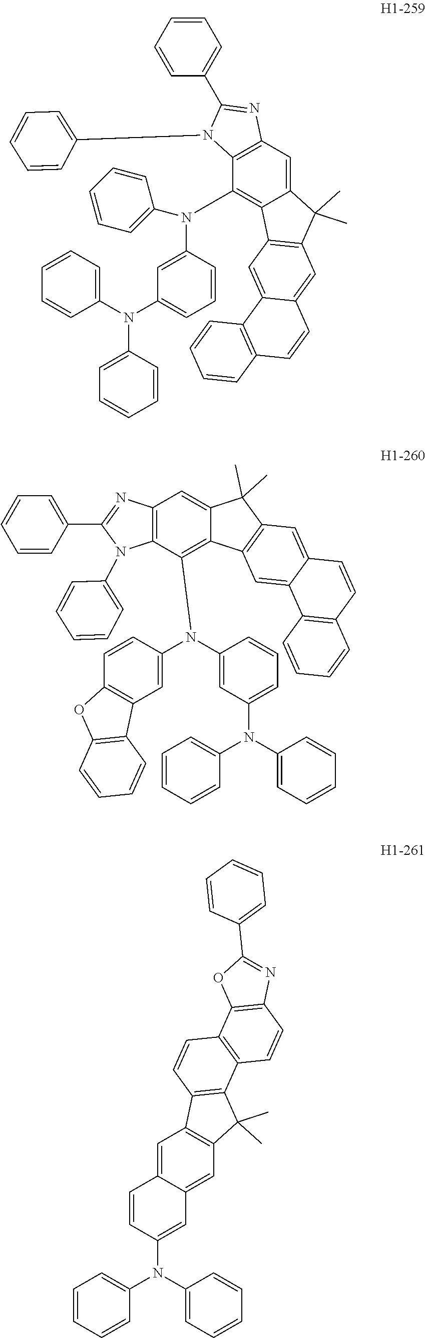 [SMILES: CC1(C)c2cc3ccc4ccccc4c3cc2-c2c1cc1nc(-c3ccccc3)n(-c3ccccc3)c1c2N(c1ccccc1)c1cccc(N(c2ccccc2)c2ccccc2)c1 ; CC1(C)c2cc3ccc4ccccc4c3cc2-c2c1cc1nc(-c3ccccc3)n(-c3ccccc3)c1c2N(c1cccc(N(c2ccccc2)c2ccccc2)c1)c1ccc2oc3ccccc3c2c1 ; CC1(C)c2cc3cc(N(c4ccccc4)c4ccccc4)ccc3cc2-c2ccc3c(ccc4nc(-c5ccccc5)oc43)c21]

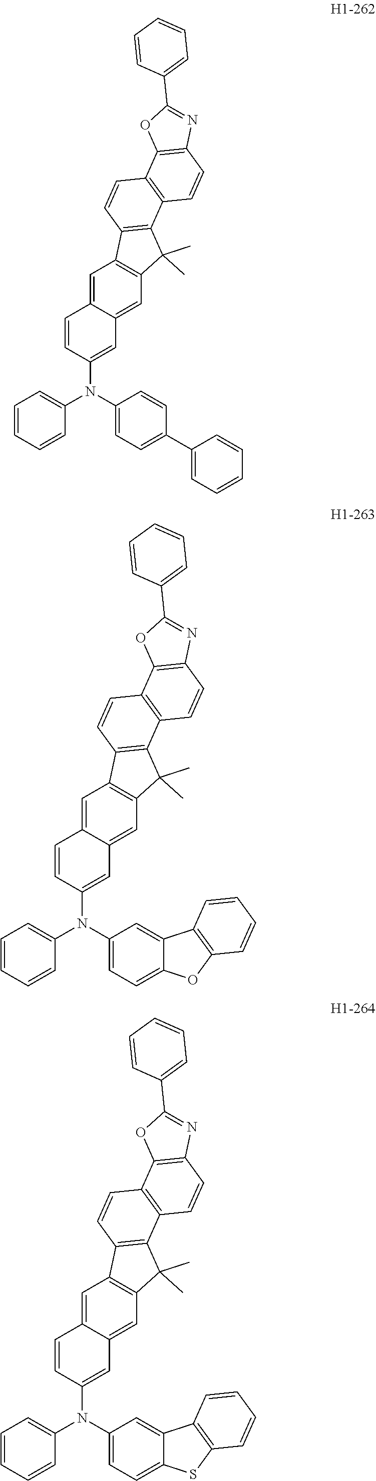

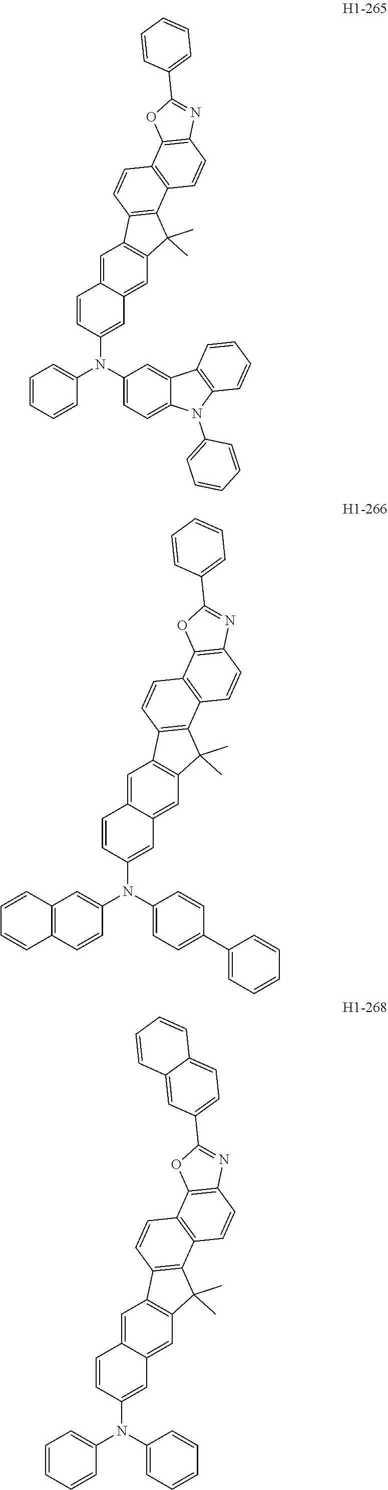

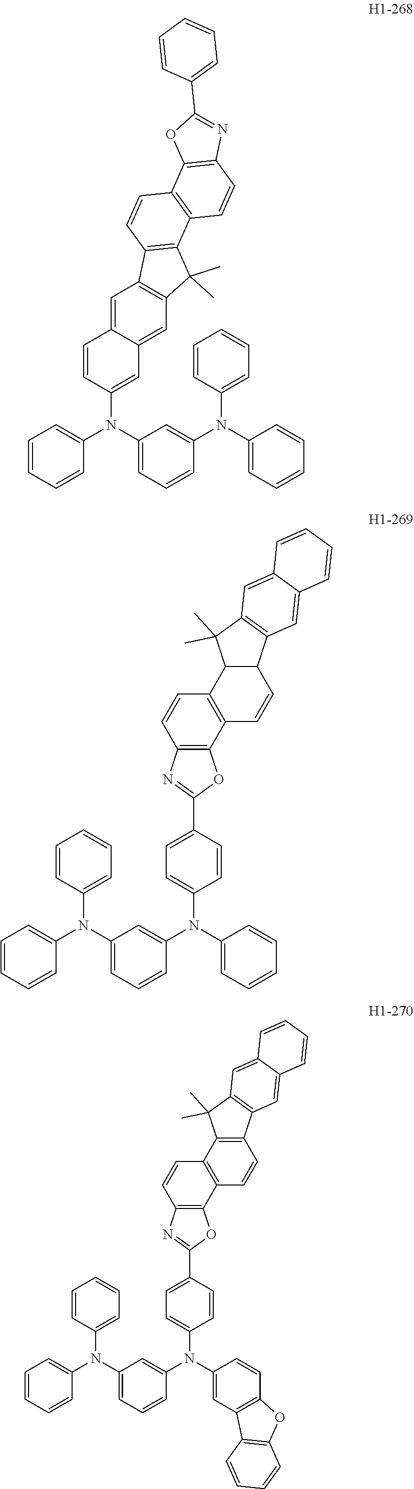 [SMILES: CC1(C)c2cc3cc(N(c4ccccc4)c4cccc(N(c5ccccc5)c5ccccc5)c4)ccc3cc2-c2ccc3c(ccc4nc(-c5ccccc5)oc43)c21 ; CC1(C)c2cc3ccccc3cc2C2C=Cc3c(ccc4nc(-c5ccc(N(c6ccccc6)c6cccc(N(c7ccccc7)c7ccccc7)c6)cc5)oc34)C21 ; CC1(C)c2cc3ccccc3cc2-c2ccc3c(ccc4nc(-c5ccc(N(c6cccc(N(c7ccccc7)c7ccccc7)c6)c6ccc7oc8ccccc8c7c6)cc5)oc43)c21]

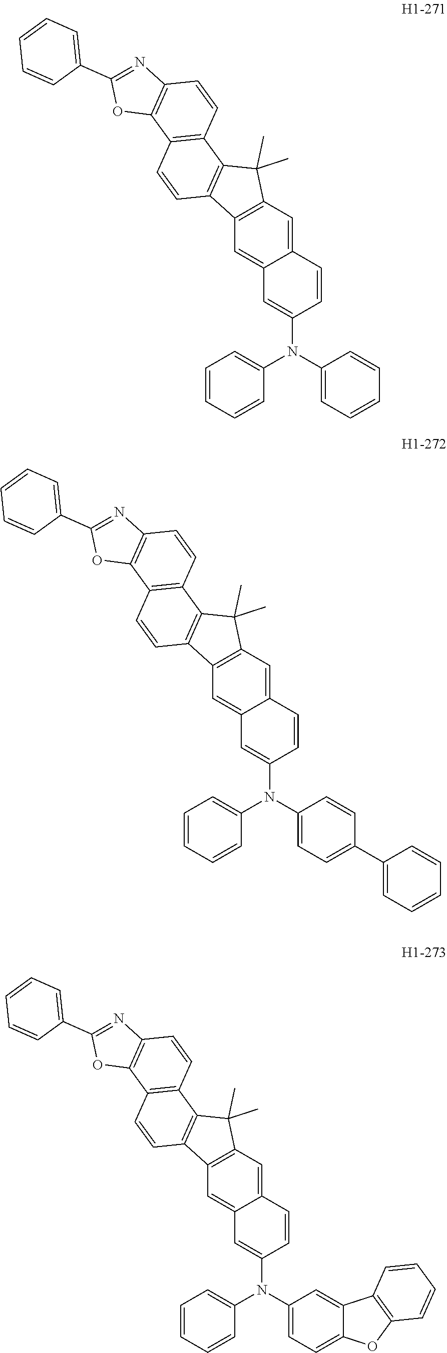 [SMILES: CC1(C)c2cc3ccc(N(c4ccccc4)c4ccccc4)cc3cc2-c2ccc3c(ccc4nc(-c5ccccc5)oc43)c21 ; CC1(C)c2cc3ccc(N(c4ccccc4)c4ccc(-c5ccccc5)cc4)cc3cc2-c2ccc3c(ccc4nc(-c5ccccc5)oc43)c21 ; CC1(C)c2cc3ccc(N(c4ccccc4)c4ccc5oc6ccccc6c5c4)cc3cc2-c2ccc3c(ccc4nc(-c5ccccc5)oc43)c21]

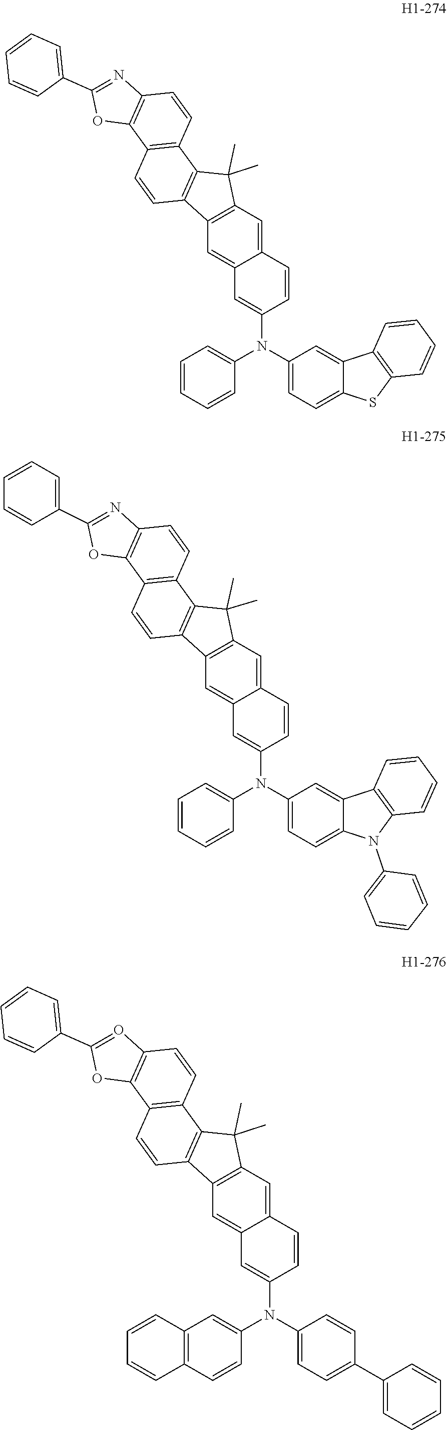

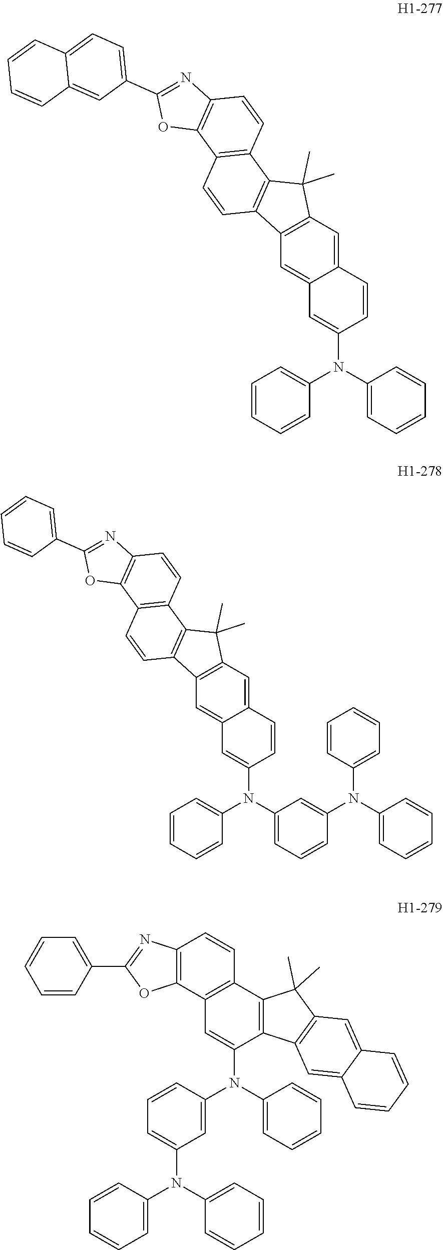 [SMILES: CC1(C)c2cc3ccc(N(c4ccccc4)c4ccccc4)cc3cc2-c2ccc3c(ccc4nc(-c5ccc6ccccc6c5)oc43)c21 ; CC1(C)c2cc3ccc(N(c4ccccc4)c4cccc(N(c5ccccc5)c5ccccc5)c4)cc3cc2-c2ccc3c(ccc4nc(-c5ccccc5)oc43)c21 ; CC1(C)c2cc3ccccc3cc2-c2c(N(c3ccccc3)c3cccc(N(c4ccccc4)c4ccccc4)c3)cc3c(ccc4nc(-c5ccccc5)oc43)c21]

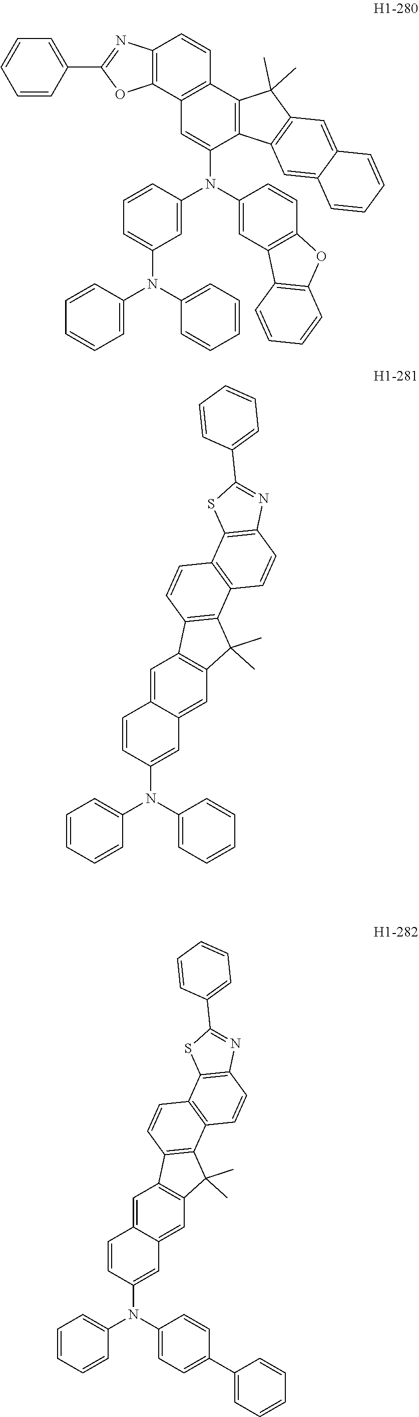

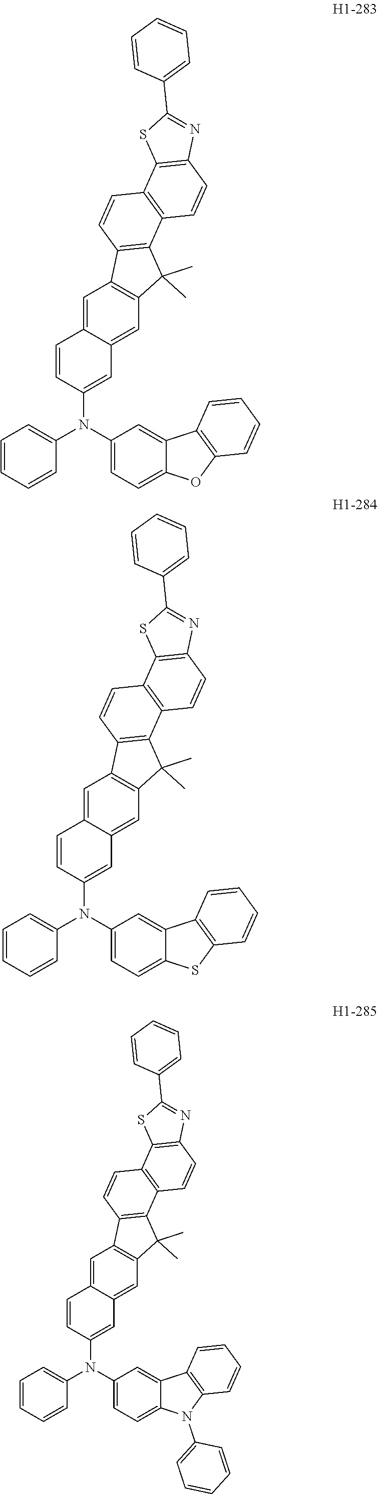

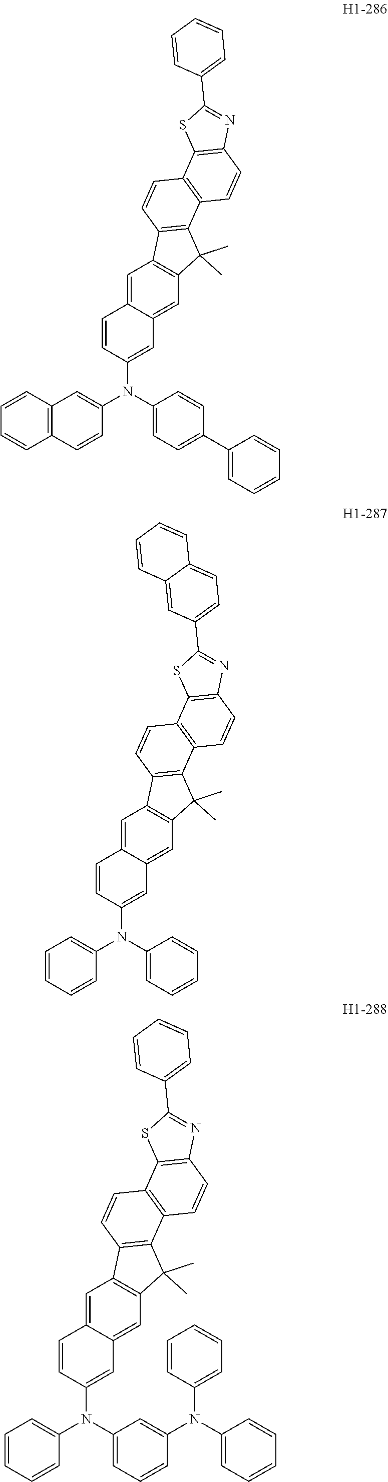

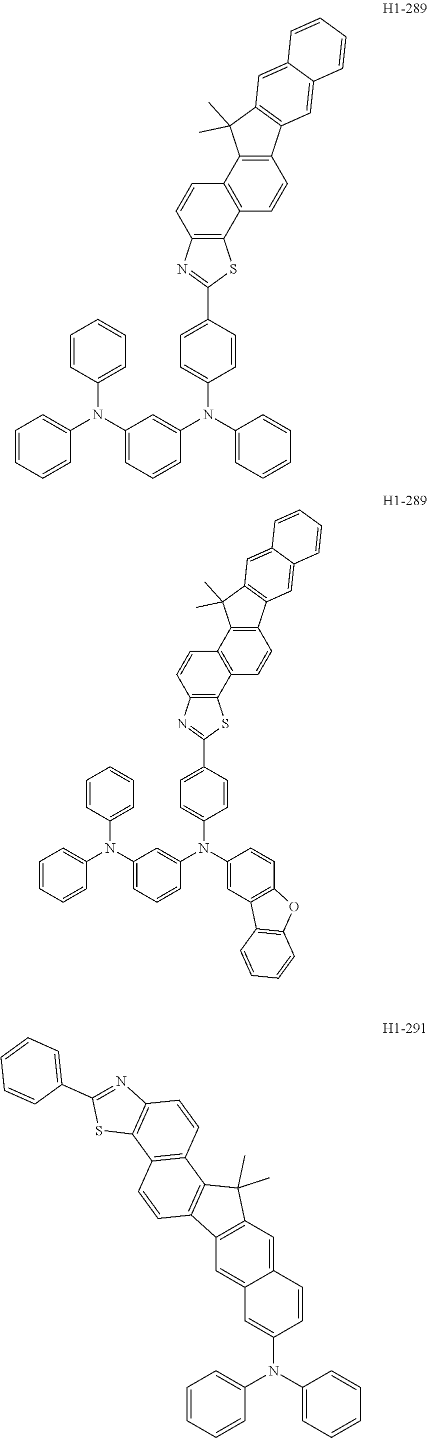

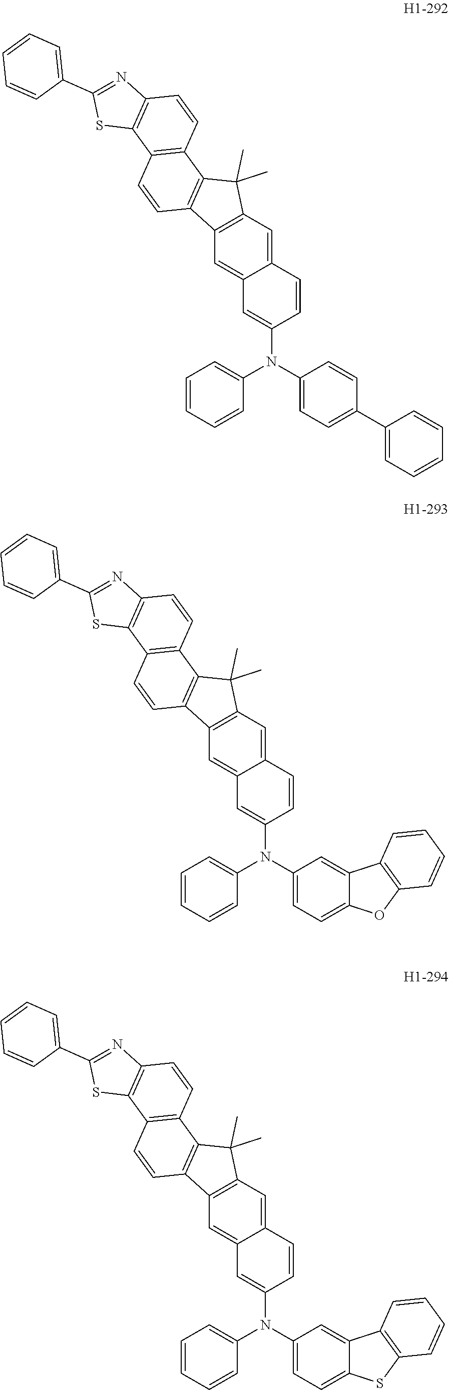 [SMILES: CC1(C)c2cc3ccc(N(c4ccccc4)c4ccc(-c5ccccc5)cc4)cc3cc2-c2ccc3c(ccc4nc(-c5ccccc5)sc43)c21 ; CC1(C)c2cc3ccc(N(c4ccccc4)c4ccc5oc6ccccc6c5c4)cc3cc2-c2ccc3c(ccc4nc(-c5ccccc5)sc43)c21 ; CC1(C)c2cc3ccc(N(c4ccccc4)c4ccc5sc6ccccc6c5c4)cc3cc2-c2ccc3c(ccc4nc(-c5ccccc5)sc43)c21]

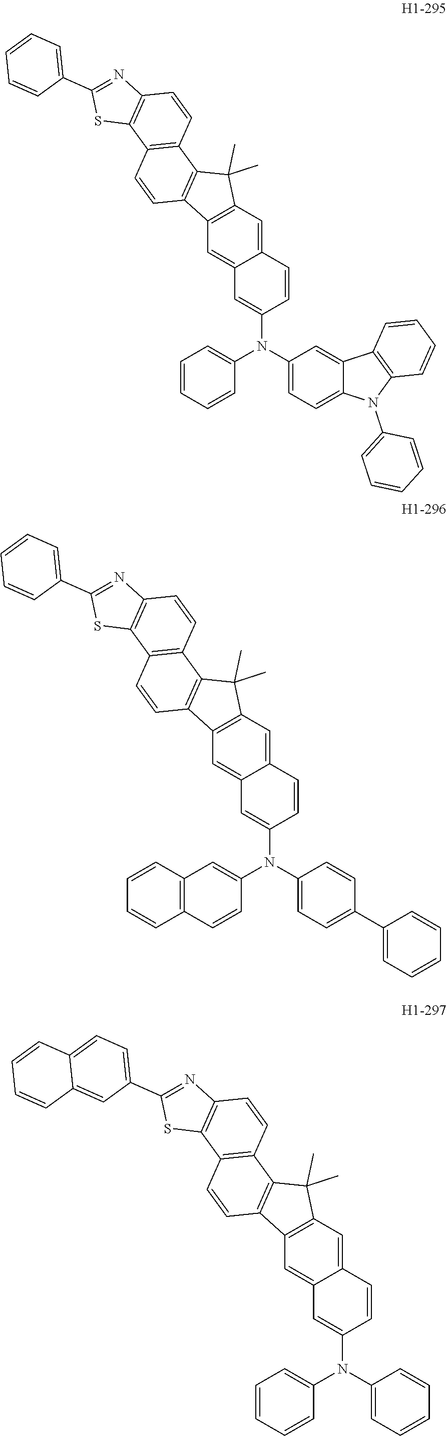 [SMILES: CC1(C)c2cc3ccc(N(c4ccccc4)c4ccc5c(c4)c4ccccc4n5-c4ccccc4)cc3cc2-c2ccc3c(ccc4nc(-c5ccccc5)sc43)c21 ; CC1(C)c2cc3ccc(N(c4ccc(-c5ccccc5)cc4)c4ccc5ccccc5c4)cc3cc2-c2ccc3c(ccc4nc(-c5ccccc5)sc43)c21 ; CC1(C)c2cc3ccc(N(c4ccccc4)c4ccccc4)cc3cc2-c2ccc3c(ccc4nc(-c5ccc6ccccc6c5)sc43)c21]

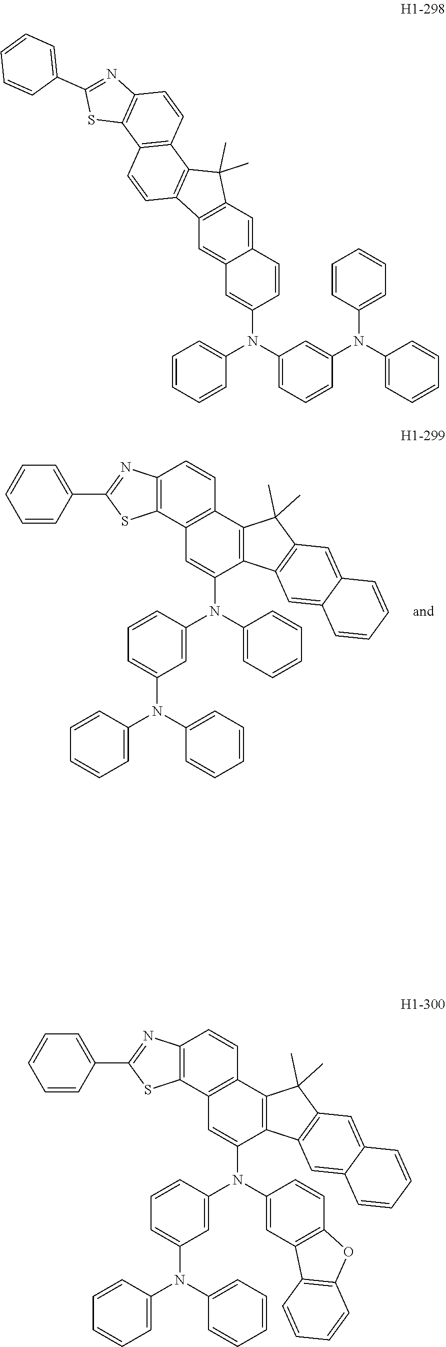 [SMILES: CC1(C)c2cc3ccc(N(c4ccccc4)c4cccc(N(c5ccccc5)c5ccccc5)c4)cc3cc2-c2ccc3c(ccc4nc(-c5ccccc5)sc43)c21 ; CC1(C)c2cc3ccccc3cc2-c2c(N(c3ccccc3)c3cccc(N(c4ccccc4)c4ccccc4)c3)cc3c(ccc4nc(-c5ccccc5)sc43)c21 ; CC1(C)c2cc3ccccc3cc2-c2c(N(c3cccc(N(c4ccccc4)c4ccccc4)c3)c3ccc4oc5ccccc5c4c3)cc3c(ccc4nc(-c5ccccc5)sc43)c21]

11. An organic electroluminescent material comprising the organic electroluminescent compound according to claim 8.

12. An organic electroluminescent device comprising the organic electroluminescent compound according to claim 8.
